SEQUENCE LISTING (76 sequences)

SEQ ID NO: 1 - protein, 248 aa, Artificial Sequence
  Sequences engineered based on Cutibacterium acnes DNA translations
MHAVEPTTTISATSTHELSASDARNSIQLLNAHIATLQSVQKSVPGSDYSDQIRDLLKAAFDLRGLIETLAHGGIPFYDPSTIMPRIKLVATTIDTIHTATTTLQNKVRPAHVELGLEVTKAVLLTANPASTAKELDAEGAALKARLEKVSQYPDLTPNDVATVYVRTNFSKTIWQVRANRDRYILGHKSAAVYKTLNHAITKAVGVRLNPKTTVGNIQAARTELLAAYQTAFNSPDVKKAAHHHHHH

SEQ ID NO: 2 - protein, 341 aa, Artificial Sequence
  Sequences engineered based on Cutibacterium acnes DNA translations
MTVKVGINGFGRIGRNFFRALAEQGADLEVVAVNDLTDTKTLAHLLKYDSIMGRFSGEVSYDDDSITVDGKTIKVLAERNPADLPWKELGAEIVVESTGLFTDGEKAKAHFEAGAKKVIISAPGKNVDGTFVMGVNDGDYDNAKHNIISNASCTTNCLAPLAKVLNDAFGIERGIMTTVHAYTGDQRLQDAPHKDLRRARAAALNMIPTKTGAAQAVALVLPELKGKFDGLAVRVPTPTGSLTDLTFQTSKETTVEEVQAAVKKAAEGPLKGILAYTEDPIVSKDIEGDPHSSIFDATETKAIGNLVKVLSWYDNEWGYSNRLVDLTKLVASKLAHHHHHH

SEQ ID NO: 3 - protein, 238 aa, Artificial Sequence
  Sequences engineered based on Cutibacterium acnes DNA translations
MSSELVDTTEMYLRTFYELLEEGVELRRARIVERLHQSGPTVSQTVARMERDGLVVVESDRSISLTQEGHDVAQTVMRKHRLAECLLTQIIGLRPDLVHDEACRWEHVISGEVEKRLTGLLENPDVSPYGCPLPPEQAGCRPDGSARFRDDSKPLDEVIAETGCPVSVTVTRLSEFFQATEGNLADVYAAGLLPGKSVEVDDDADGIRLTGPSGSIVVDPEILSGLFVAQNSHHHHHH

SEQ ID NO: 4 - protein, 411 aa, Artificial Sequence
  Sequences engineered based on Cutibacterium acnes DNA translations
MHHHHHHLSLLRRLLRWSDQSQQSPPPPPPQAEASSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPTPTPKPTPTPTPKPTPTPKPKPTPAPAPTSGATSDESTSRSGGHSQGGSGTHYIHHGVAPVLTHSSDLPSTGF

SEQ ID NO: 5 - protein, 425 aa, Artificial Sequence
  Sequences engineered based on Cutibacterium acnes DNA translations
MHHHHHHDSQIRGEAMRRSRIAVAAATATALVGSIAAITPSPQAEASSNRPRSVAQAAIATDGKGIINKDCRDAVINDAKLRAAIAGALVKAGFSSADAVALAPRIAREMAKEGVLLINHHKLKALIGAQVGLLTDAKIQRAAAAVDLGIKATLAATIIPNALHSAAFKDAVVANLVAAGVDKKLAKATAVAIAATALNPALGPIAKTEAIKAEIGAQAALLVSRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPTPTPTPKPTPTPTPTPTPTPKPKPTPAPAPTSGATSDESTSRSGGHSQGGSGTHYIHHGVAPVLTHSSDLPSTGF

SEQ ID NO: 6 - protein, 384 aa, Artificial Sequence
  Sequences engineered based on Cutibacterium acnes DNA translations
MHHHHHHSSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPTPTPKPTPTPTPKPTPTPKPKPTPAPAPTSGATSDESTSRSGGHSQGGSGTHYIHHGVAPVLTHSSDLPSTGF

SEQ ID NO: 7 - protein, 378 aa, Artificial Sequence
  Sequences engineered based on Cutibacterium acnes DNA translations
MSSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPTPTPKPTPTPTPKPTPTPKPKPTPAPAPTSGATSDESTSRSGGHSQGGSGTHYIHHGVAPVLTHSSDLPSTGF

SEQ ID NO: 8 - protein, 439 aa, Artificial Sequence
  Sequences engineered based on Cutibacterium acnes DNA translations
MITKFPTPRRNDASNKNHCGSCDSNCLNRLTISYHPLPANAASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPTPTPTPTPTPTPAPTPTPAPTPTPAPTPAPTPTPAPTPTPTPTPTPTPTPTHGATTTTPISRTTDRHNLGSHHTRIAAPALIHAKALPATGTGAHHHHHH

SEQ ID NO: 9 - protein, 469 aa, Artificial Sequence
  Sequences engineered based on Cutibacterium acnes DNA translations
MIIFVRVGNRHMRKRRAVLVVAFQLVNRRLAITKFPTPRRNDASNKNHCGSCDSNCLNRLTISYHPLPANAASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPTPTPTPTPTPTPAPTPTPAPTPTPAPTPAPTPTPAPTPTPTPTPTPTPTPTHGATTTTPISRTTDRHNLGSHHTRIAAPALIHAKALPATGTGAHHHHHH

SEQ ID NO: 10 - protein, 462 aa, Artificial Sequence
  Sequences engineered based on Cutibacterium acnes DNA translations
MHHHHHHLSPSFQPLGETMRRTRITVAAVTATALIGSLSAITPLPANAASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPTPTPTPTPTPTPTPTPTPTPTPTPTPTPTPAPTPAPTPAPTPTPTPTPAPTPTPTPTPTPTPTPTPTPTPTPTHGATTTTPISRTTDRHNLGSHHTRIAAPALIHAKALPATGTGA

SEQ ID NO: 11 - protein, 399 aa, Artificial Sequence
  Sequences engineered based on Cutibacterium acnes DNA translations
MHHHHHHASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPTPTPTPTPTPTPAPTPTPAPTPTPAPTPAPTPTPAPTPTPTPTPTPTPTPTHGATTTTPISRTTDRHNLGSHHTRIAAPALIHAKALPATGTGA

SEQ ID NO: 12 - protein, 393 aa, Artificial Sequence
  Sequences engineered based on Cutibacterium acnes DNA translations
MASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPTPTPTPTPTPTPAPTPTPAPTPTPAPTPAPTPTPAPTPTPTPTPTPTPTPTHGATTTTPISRTTDRHNLGSHHTRIAAPALIHAKALPATGTGA

SEQ ID NO: 13 - protein, 443 aa, Artificial Sequence
  Sequences engineered based on Cutibacterium acnes DNA translations
MHHHHHHAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTGAEGPSSDEIDLGIVGGLALTAVVSTVVVCRRYAARI

SEQ ID NO: 14 - protein, 400 aa, Artificial Sequence
  Sequences engineered based on Cutibacterium acnes DNA translations
MAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 15 - protein, 437 aa, Artificial Sequence
  Sequences engineered based on Cutibacterium acnes DNA translations
MAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTGAEGPSSDEIDLGIVGGLALTAVVSTVVVCRRYAARI

SEQ ID NO: 16 - protein, 295 aa, Artificial Sequence
  Sequences engineered based on Cutibacterium acnes DNA translations
MAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLS

SEQ ID NO: 17 - protein, 361 aa, Artificial Sequence
  Sequences engineered based on Cutibacterium acnes DNA translations
MAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLS

SEQ ID NO: 18 - protein, 400 aa, Artificial Sequence
  Sequences engineered based on Cutibacterium acnes DNA translations
MAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVSTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVT

SEQ ID NO: 19 - protein, 400 aa, Artificial Sequence
  Sequences engineered based on Cutibacterium acnes DNA translations
MAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVSHNVTVDAVTGKVIGDDSGKGAGRGLPVT

SEQ ID NO: 20 - protein, 400 aa, Artificial Sequence
  Sequences engineered based on Cutibacterium acnes DNA translations
MAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVSTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVSHNVTVDAVTGKVIGDDSGKGAGRGLPVT

SEQ ID NO: 21 - protein, 970 aa, Artificial Sequence
  Sequences engineered based on Cutibacterium acnes DNA translations
MAKVRVYELAKELGLSSKQLLGKLNDMGEFVRSASSTIEAPVVRRLRDQIGSAEPADDAKAAKPAARKSQTSSKKTSKETTTAKPAPGPKPGPGPKPTPGPRPGSSSGPKPGRSSAARTSATPRPGLIKSSGASSQQEPPAAKPESKPTPKPGQNVPKPHAPAPKPKPGAPSKSPKPGARPGPRPGGSAGLPSAARPGPRPGAGRRTGAPRPGNNPFASSQGMGQSRHRSEGGQRSGGSRSGQGERMPRPGGSQGSRGGSGMPRPNPAMMPKHQSSQIGQATTGRGGRGGGRGRGGSRGSGFGGGFGGGPRGPIGGGRGGRGGRGTQGAFGRGGGGRRGRKSRKQRRQEFDEMQAPLVGGVRVRKGNGETVRLRRGASLTDLAEKINAEPAQLVQVLFNLGEMVTATQSVSDDTLEILGGELNYQIQVVSPEDEDRELLESFDLEFGEDEGDDSDLVARPPVVTVMGHVDHGKTKLLDALRHTDVVKGEAGGITQAIGAYQVQTEVDDAERAITFIDTPGHEAFTAMRARGAQSTDIAVLVVAADDGVMPQTVEALNHAKAADVPIVVAVNKIDKPEADPDKVRGQLTEYGLVPEEYGGDTMFVNVSARTHEGLDDLLEAIVLTADAALDLRANPDMAAQGVAIEAHLDKGRGPVATALIQRGTLHIGDSIVAGSSYGRVRAMINDQGESVDEAAPATPVQVLGLTSVPGAGDNFLVVDDDRKARQIAEKREARMRAAQQARSSRRKTLDQLFEQLEKGETEELLLILKGDGAGSVEALEDALAKIDVGDEVDLRVIDRGVGAITETNVSLAAASNAVIVGFNVRPTAHAQRMADEENVDIRYYSVIYDAIDEIEAALRGMLKPIYEEKAMGTAEIRQIFRSSKVGTIAGCMITDGTIRRHAKARLVRDGVVVQETEINTLQREKDAVTEVREGYECGLTLTNYSDIHVGDEVQCYEMVEKPRDHHHHHH

SEQ ID NO: 22 - protein, 333 aa, Artificial Sequence
  Sequences engineered based on Cutibacterium acnes DNA translations
MTQTPVKIAVTGAAGQICYSLLFRIASGSLLGDTPIELRLLEITPALKALEGVVMELDDCAFGNLVNIEIGDDPKKVFDGVNAAFLVGAMPRKAGMERSDLLTKNGAIFAAQGKALNDVAADDVRVLVTGNPANTNALIAATNAVDIPNNHFAALTRLDHNRAKTQLARKTGKTVNDVRHMTIWGNHSSTQYPDVFHAEVAGQKATNLVNEAWIENEFIPTVAKRGAAIIDARGASSAASAANATVECMRDWMGSTPEGDWVSMAIPSDGSYGVPEGLISSFPVTITNGKVEIVQGLDIDDFSRAKIDASAKELADERDAVKELGLIHHHHHH

SEQ ID NO: 23 - protein, 441 aa, Artificial Sequence
  Sequences engineered based on Cutibacterium acnes DNA translations
MHHHHHHDAVNEIHAKAGDQASVSLTAYADSPFYKKLPSYVSDSLRDGLFYYTGYYLEDGAAAGLANLTALHPTDTQDNSHTLDGDPVLAYCIEKNSLQERYHQDTGTLTKWSELKPHYNDNLTNALPKIGWIAKNGYPSKALDELEKQANAANGNDDATGSDHRDAVTATQAAIWHFSDDFTLKSDDITEYENGRLISVPDSAKKHISSLYNYLIKKAVPVSDPLDSTSDARGTSDFAGAALSFEAGQRGHQDRIIVPIQPISPAPTPTPTPTPTPTPTPTPTPTPTPTPTPTPTPTPTVDVKVTTTADQTAEVGKPFGDTAHITGTVPEGAHITFKLSHKGLLPNMCGDSVITTKPVAVPAGNHIQPIDIASPKVTVGQAGTYYWVETLTDSNGKVLAQGKCGEHAETTTVTAPVVPTPTPTMIPVGPSHPGLMPHTGV

SEQ ID NO: 24 - protein, 349 aa, Artificial Sequence
  Sequences engineered based on Cutibacterium acnes DNA translations
MHHHHHHTNTSTNAGTTLKRTVALAAAASLAVMGTIAEEAHAAPAGNLGAHAEKAPTQHTKKAPAEQAKRATTYTARFALTRVHLNVRSGHSTEYRRYGLLRPGDKLLIIGEDVRGWTPVNYRGKTAWVATRYITKMNRPVGIYAQRGDHNARSKAVQRDLSTLNYFPAKWVGLPYGPITTNAVKVFQRANALKQTGVTDAVTLGLLKSKADYQRAAAAEKSSTYKHAEHHSEHSSSTSRSTARPARNTSPDVSTSGIAGSCQASFYDDSQTASGESFSSSALTAASKTYSFGTRLKVTNKANGQSVVVRVNDRGPYVSGRCLDLSSAAFSRISSLSAGVADVTYAKVS

SEQ ID NO: 25 - protein, 146 aa, Artificial Sequence
  Sequences engineered based on Cutibacterium acnes DNA translations
MHHHHHHATPIDESQLPVGPQVSVTDSAQHTGPFAASSPLTITVKPGAPCVRADGYQESMVTRVLDDKGHQVWTGTFDESKLIGGTGLGTATFHVGSPAAAFNFHGSERTTYRTLSYCAYPHYVNGTRERLSQVSVKTFMVDPALN

SEQ ID NO: 26 - protein, 282 aa, Artificial Sequence
  Sequences engineered based on Cutibacterium acnes DNA translations
MHHHHHHKNTGRDSAADGKTTVKIGTTEAANDYWNVIKKKAAAEGINIDVVSFNDYTQPNVALSQGQIDLNAFQHLLFLADYDVKNNDNITALAATYVVPLNIYSKKYQRLSQLPAGATIAIPNDATNQGRALLVLQEAGLLKLRGNGSALSTPADVIANQSKVKVKAIDAAQTAASLEGVDGAVVNNNFATDAGLDRNKVLYHDDATGKSADPYLNIIAVKDADKDNPTYQKVAQLWSDPDVTKAIIKQSGGTAKIVSGHSKADLDKILSGLTTDIKKSNS

SEQ ID NO: 27 - protein, 250 aa, Artificial Sequence
  Sequences engineered based on Cutibacterium acnes DNA translations
MHHHHHHSASVTAATTMTSRTASAVRTLDLTKTYGSGTTLVRALRSVTLDIPSGRFTAIMGASGSGKSTLLHCITGLDSSTSGRVWIGDTEITHLDDDALTKLRRQHVGFVFQSFNLMPTLSAADNITLPLRLSHTKVDKEWFDHITGMLGITERLGHKPSQMSGGQQQRVAVARALVTRPDVIVADEPTGALDSEASGDLLTFLRHCVDDLGQTVVMVTHDHDAAARADDIVTVTDGQVSTARSQEALA

SEQ ID NO: 28 - protein, 497 aa, Artificial Sequence
  Sequences engineered based on Cutibacterium acnes DNA translations
MHHHHHHDQPDADQSQRQVALGAMQWTINSHFLNALKMMMHGSWSTSSGASWNGKAFSFPVSKGSLSSTAQKADVKYSGSVHFTGVGGRMDLTLADPEIVVNNGDDHVIMSVKSKTMVGQSVDYGRITFVDMTGSITQGQNDAAQVVGTNVKLNSQAVDAFAGFYQAGKPMDDIDSSLKLGPQSSRPEPTVTPKPSVTPKPSVKPKPTVAPKPSVKPKPRVSPKPSAKPKPTVAPKPDQSTIMAPQSDAATEAAAGEAPSAGAHKIIRKQVCTVVPGASRATSPTSTPAKVGKGSMNWGVSTSFVNALKGPMQGKWTMSGGASADGTRFNFPFSGGNYDRATKTGNLNFSGGVHFTGVHGLLDLSLSDPSLSLKNGKGVMKLTLNAGGMAPFKGRVDFANVSVVPSGSGFTATSVVLTGNGVQVWNGFYKKGHSMDNFSVSLGDKSDSASAAKQNSASLVKKSAASPTKRECHDVLVDAKTGQIISGSNGSLPNTGV

SEQ ID NO: 29 - protein, 148 aa, Artificial Sequence
  Sequences engineered based on Cutibacterium acnes DNA translations
MHHHHHHLSLLRRLLRWSDQSQQSPPPPPPQAEASSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIP

SEQ ID NO: 30 - protein, 148 aa, Artificial Sequence
  Sequences engineered based on Cutibacterium acnes DNA translations
MHHHHHHGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSP

SEQ ID NO: 31 - protein, 163 aa, Artificial Sequence
  Sequences engineered based on Cutibacterium acnes DNA translations
MHHHHHHGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPTPTPKPTPTPTPKPTPTPKPKPTPAPAPTSG

SEQ ID NO: 32 - protein, 142 aa, Artificial Sequence
  Sequences engineered based on Cutibacterium acnes DNA translations
MHHHHHHPLLALNTAKLMKNITRQIVDVITADKAIKTAEQLEKELPALDDLVKKACSCPKPTPTPTPTPTPTPKPTPTPTPKPTPTPKPKPTPAPAPTSGATSDESTSRSGGHSQGGSGTHYIHHGVAPVLTHSSDLPSTGF

SEQ ID NO: 33 - protein, 242 aa, Artificial Sequence
  Sequences engineered based on Cutibacterium acnes DNA translations
MHHHHHHLSLLRRLLRWSDQSQQSPPPPPPQAEASSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSP

SEQ ID NO: 34 - protein, 369 aa, Artificial Sequence
  Sequences engineered based on Cutibacterium acnes DNA translations
MHHHHHHLSLLRRLLRWSDQSQQSPPPPPPQAEASSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPTPTPKPTPTPTPKPTPTPKPKPTPAPAPTSG

SEQ ID NO: 35 - protein, 317 aa, Artificial Sequence
  Sequences engineered based on Cutibacterium acnes DNA translations
MHHHHHHGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPTPTPKPTPTPTPKPTPTPKPKPTPAPAPTSGATSDESTSRSGGHSQGGSGTHYIHHGVAPVLTHSSDLPSTGF

SEQ ID NO: 36 - protein, 305 aa, Artificial Sequence
  Sequences engineered based on Cutibacterium acnes DNA translations
MHHHHHHPPQAEASSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACS

SEQ ID NO: 37 - protein, 312 aa, Artificial Sequence
  Sequences engineered based on Cutibacterium acnes DNA translations
MHHHHHHSSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPT

SEQ ID NO: 38 - protein, 124 aa, Artificial Sequence
  Sequences engineered based on Cutibacterium acnes DNA translations
MHHHHHHSSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNAL

SEQ ID NO: 39 - protein, 138 aa, Artificial Sequence
  Sequences engineered based on Cutibacterium acnes DNA translations
MHHHHHHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNT

SEQ ID NO: 40 - protein, 177 aa, Artificial Sequence
  Sequences engineered based on Cutibacterium acnes DNA translations
MHHHHHHKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPTPTPKPTPTPTPKPTPTPKPKPTPAPAPTSG

SEQ ID NO: 41 - protein, 219 aa, Artificial Sequence
  Sequences engineered based on Cutibacterium acnes DNA translations
MHHHHHHKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPTPTPKPTPTPTPKPTPTPKPKPTPAPAPTSGATSDESTSRSGGHSQGGSGTHYIHHGVAPVLTHSSDLPSTGF

SEQ ID NO: 42 - protein, 299 aa, Artificial Sequence
  Sequences engineered based on Cutibacterium acnes DNA translations
MHHHHHHSSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACS

SEQ ID NO: 43 - protein, 139 aa, Artificial Sequence
  Sequences engineered based on Cutibacterium acnes DNA translations
MHHHHHHGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNT

SEQ ID NO: 44 - protein, 273 aa, Artificial Sequence
  Sequences engineered based on Cutibacterium acnes DNA translations
MHHHHHHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNT

SEQ ID NO: 45 - protein, 242 aa, Artificial Sequence
  Sequences engineered based on Cutibacterium acnes DNA translations
MHHHHHHSSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNAL

SEQ ID NO: 46 - protein, 312 aa, Artificial Sequence
  Sequences engineered based on Cutibacterium acnes DNA translations
MHHHHHHSSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKACSCPKPTPTPTPTPT

SEQ ID NO: 47 - protein, 306 aa, Artificial Sequence
  Sequences engineered based on Cutibacterium acnes DNA translations
MSSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKACSCPKPTPTPTPTPT

SEQ ID NO: 48 - protein, 306 aa, Artificial Sequence
  Sequences engineered based on Cutibacterium acnes DNA translations
MSSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKACSCPKPTPTPTPTPT

SEQ ID NO: 49 - protein, 306 aa, Artificial Sequence
  Sequences engineered based on Cutibacterium acnes DNA translations
MSSNRPRSVAQAAIATDGKGIIDKDSRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKASSPPKPTPTPTPTPT

SEQ ID NO: 50 - protein, 195 aa, Artificial Sequence
  Sequences engineered based on Cutibacterium acnes DNA translations
MHHHHHHGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKACSCPKPTPTPTPTPT

SEQ ID NO: 51 - protein, 17 aa, Propionibacterium acnes
SDTDTDSNPNADADTDA

SEQ ID NO: 52 - protein, 10 aa, Propionibacterium acnes
TTPQQKTAEH

SEQ ID NO: 53 - protein, 48 aa, Propionibacterium acnes
DAKIQRAAAAVDLGIKATLAATIIPNALHSAAFKDAVVANLVAAGVDK

SEQ ID NO: 54 - protein, 48 aa, Propionibacterium acnes
DAAVQHAAAAIDLSFKAILGASIIPNALGSAAFKNAVIANLVAAGIDK

SEQ ID NO: 55 - protein, 48 aa, Artificial Sequence
  chimeric peptide
DAKIQRAAAAVDLGIKATLAATIIPNALGSAAFKNAVIANLVAAGIDK

SEQ ID NO: 56 - protein, 46 aa, Propionibacterium acnes
AVQIAPRIIDRLAKEPLLALNTAKLMKNITRQIVDVITADKAIKTA

SEQ ID NO: 57 - protein, 46 aa, Propionibacterium acnes
AISLAPHIVKRLAKEPLLAFNTAKLVKDIARQIVDIRNTQEAIAVY

SEQ ID NO: 58 - protein, 46 aa, Artificial Sequence
  chimeric peptide
AISLAPHIVKRLAKEPLLAFNTAKLMKNITRQIVDVITADKAIKTA

SEQ ID NO: 59 - protein, 12 aa, Propionibacterium acnes
  Xaa (2)..(2): Asp or Gly
  Xaa (5)..(5): Lys or Gln
  Xaa (9)..(9): Ser or Thr
  Xaa (10)..(10): Cys present or not
  Xaa (12)..(12): Lys, Asp or Glu
DXLVXKACXXPX

SEQ ID NO: 60 - protein, 5 aa, Propionibacterium acnes
  Xaa (3)..(3): Xaa can be any naturally occurring amino acid
LPXTG

SEQ ID NO: 61 - protein, 305 aa, Propionibacterium acnes
SSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPT

SEQ ID NO: 62 - protein, 304 aa, Propionibacterium acnes
SNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPTPTPTPT

SEQ ID NO: 63 - protein, 305 aa, Propionibacterium acnes
SSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLMKNITRQIVDVITADKAIKTAEQLEKELPALDDLVKKACSCPKPTPTPTPTPT

SEQ ID NO: 64 - protein, 405 aa, Propionibacterium acnes
MLSLLRRLLRWSDQSQQSPPPPPPQAEASSNRPRSVAQAAIATDGKGIIDKDCRDAVINDAKLRAAIAGALVKAGFSSADAVALAPRIAKEMAKEGVLLINHHKLKALIGAQLGLLTDAKIQRAAAAVDLGIKATLAATIIPNALHSAAFKDAVVANLVAAGVDKKLAKATAVAIAATALNPALGPIAKTEAIKAEIAAQAALLVGRGVHLKKAAIEHIIGRSFDAAVATAIVSSPILNARIVTHLVRAGIDKSLAVQIAPRIIDRLAKEPLLALNTAKLMKNITRQIVDVITADKAIKTAEQLEKELPALDDLVKKACSCPKPTPTPTPTPTPTPKPTPTPTPKPTPTPKPKPTPAPAPTSGATSDESTSRSGGHSQGGSGTHYIHHGVAPVLTHSSDLPSTGF

SEQ ID NO: 65 - protein, 463 aa, Propionibacterium acnes
MIIFVRVGNRHMRKRRAVLVVAFQLVNRRLAITKFPTPRRNDASNKNHCGSCDSNCLNRLTISYHPLPANAASNGNSSITQSAAFSPRATTKISEDCRKAIINDLKLRGAIVGALVKAGLSAADAAALAPRIAAEMAAEGTLTINHHRLKVLVASQLGLVADAAVQHAAAAIDLSFKAILGASIIPNALGSAAFKNAVIANLVAAGIDKHLARATAVAIVATALNPALGPIAKFELIKAEIAAQAALLIRRGVHLQKAAIEHVIGRAFDAAVATAIISSPILSARIVTHLVRAGIDKSIAISLAPHIVKRLAKEPLLAFNTAKLVKDIARQIVDIRNTQEAIAVYKQLKAELPTLDGLVQKACTPEPTPTPTPTPTPTPTPAPTPTPAPTPTPAPTPAPTPTPAPTPTPTPTPTPTPTPTHGATTTTPISRTTDRHNLGSHHTRIAAPALIHAKALPATGTGA

SEQ ID NO: 66 - protein, 467 aa, Propionibacterium acnes
MTTSAMRKIVASVLGAILALTGVLITPAAWAAGPTVTVIPVGREGGDITISGKGFSTTGFGVYVAVAPASVPEFYGNSDKFYGYDPSKDTTESPSTIWVYTPSQKAIGSRFAQGRPMNNDGSFTITMKAPPFEQGKDFVVLTTKAHGVGKTDHSDDTRTPVTYREATPAPTGPKTPIAPSKQPSKQAAPSKQVKPSKQAGPNKQSTTPQQKTAEHRSQTPAAHRTMTKQVCTIGASKVTSGSLTWGIRTSFTSYLRGPIANGSWKLSGGANWNGSAFTFPLTSGSFDPATKSGSLKYSGSVHMTGHHGILDMTLAEPSLQIKGSTGHLYLDVKSSSMDGKKTNYGRVDFATFGVSVSGNAAIKGSPVKLTATGAKAFAGFYRAGEPMNPLSTNLTLSAEKVCHNVTVDAVTGKVIGDDSGKGAGRGLPVTGAEGPSSDEIDLGIVGGLALTAVVSTVVVCRRYAARI

SEQ ID NO: 67 - DNA, 918 bp, Propionibacterium acnes
atgagcagcaacagacccagaagcgtggcccaggccgccatcgccaccgacggcaagggcatcatcgacaaggacagcagagacgccgtgatcaacgacgccaagctgagagccgccatcgccggcgccctggtgaaggccggcttcagcagcgccgacgccgtggccctggcccccagaatcgccaaggagatggccaaggagggcgtgctgctgatcaaccaccacaagctgaaggccctgatcggcgcccagctgggcctgctgaccgacgccaagatccagagagccgccgccgccgtggacctgggcatcaaggccaccctggccgccaccatcatccccaacgccctgggcagcgccgccttcaagaacgccgtgatcgccaacctggtggccgccggcatcgacaagcacctggccagagccaccgccgtggccatcgtggccaccgccctgaaccccgccctgggccccatcgccaagttcgagctgatcaaggccgagatcgccgcccaggccgccctgctgatcagaagaggcgtgcacctgcagaaggccgccatcgagcacgtgatcggcagagccttcgacgccgccgtggccaccgccatcatcagcagccccatcctgagcgccagaatcgtgacccacctggtgagagccggcatcgacaagagcatcgccatcagcctggccccccacatcgtgaagagactggccaaggagcccctgctggccttcaacaccgccaagctgatgaagaacatcaccagacagatcgtggacgtgatcaccgccgacaaggccatcaagaccgccgagcagctggagaaggagctgcccgccctggacgacctggtgaagaaggccagcagcccccccaagcccacccccacccccacccccacccccacc

SEQ ID NO: 68 - DNA, 918 bp, Propionibacterium acnes
atgagcagcaaccggccccggagcgtggcccaggccgccatcgccaccgacggcaagggcatcatcgacaaggacagccgggacgccgtgatcaacgacgccaagctgcgggccgccatcgccggcgccctggtgaaggccggcttcagcagcgccgacgccgtggccctggccccccggatcgccaaggagatggccaaggagggcgtgctgctgatcaaccaccacaagctgaaggccctgatcggcgcccagctgggcctgctgaccgacgccaagatccagcgggccgccgccgccgtggacctgggcatcaaggccaccctggccgccaccatcatccccaacgccctgggcagcgccgccttcaagaacgccgtgatcgccaacctggtggccgccggcatcgacaagcacctggcccgggccaccgccgtggccatcgtggccaccgccctgaaccccgccctgggccccatcgccaagttcgagctgatcaaggccgagatcgccgcccaggccgccctgctgatccggcggggcgtgcacctgcagaaggccgccatcgagcacgtgatcggccgggccttcgacgccgccgtggccaccgccatcatcagcagccccatcctgagcgcccggatcgtgacccacctggtgcgggccggcatcgacaagagcatcgccatcagcctggccccccacatcgtgaagcggctggccaaggagcccctgctggccttcaacaccgccaagctgatgaagaacatcacccggcagatcgtggacgtgatcaccgccgacaaggccatcaagaccgccgagcagctggagaaggagctgcccgccctggacgacctggtgaagaaggccagcagcccccccaagcccacccccacccccacccccacccccacc

SEQ ID NO: 69 - DNA, 1200 bp, Propionibacterium acnes
atggccggccccaccgtgaccgtgatccccgtgggcagagagggcggcgacatcaccatcagcggcaagggcttcagcaccaccggcttcggcgtgtacgtggccgtggcccccgccagcgtgcccgagttctacggcaacagcgacaagttctacggctacgaccccagcaaggacaccaccgagagccccagcaccatctgggtgtacacccccagccagaaggccatcggcagcagattcgcccagggcagacccatgaacaacgacggcagcttcaccatcaccatgaaggccccccccttcgagcagggcaaggacttcgtggtgctgaccaccaaggcccacggcgtgggcaagaccgaccacagcgacgacaccagaacccccgtgacctacagagaggccacccccgcccccaccggccccaagacccccatcgcccccagcaagcagcccagcaagcaggccgcccccagcaagcaggtgaagcccagcaagcaggccggccccaacaagcagagcaccaccccccagcagaagaccgccgagcacagaagccagacccccgccgcccacagaaccatgaccaagcaggtgagcaccatcggcgccagcaaggtgaccagcggcagcctgacctggggcatcagaaccagcttcaccagctacctgagaggccccatcgccaacggcagctggaagctgagcggcggcgccaactggaacggcagcgccttcaccttccccctgaccagcggcagcttcgaccccgccaccaagagcggcagcctgaagtacagcggcagcgtgcacatgaccggccaccacggcatcctggacatgaccctggccgagcccagcctgcagatcaagggcagcaccggccacctgtacctggacgtgaagagcagcagcatggacggcaagaagaccaactacggcagagtggacttcgccaccttcggcgtgagcgtgagcggcaacgccgccatcaagggcagccccgtgaagctgaccgccaccggcgccaaggccttcgccggcttctacagagccggcgagcccatgaaccccctgagcaccaacctgaccctgagcgccgagaaggtgagccacaacgtgaccgtggacgccgtgaccggcaaggtgatcggcgacgacagcggcaagggcgccggcagaggcctgcccgtgacc

SEQ ID NO: 70 - DNA, 1200 bp, Propionibacterium acnes
atggccggccccaccgtgaccgtgatccccgtgggccgggagggcggcgacatcaccatcagcggcaagggcttcagcaccaccggcttcggcgtgtacgtggccgtggcccccgccagcgtgcccgagttctacggcaacagcgacaagttctacggctacgaccccagcaaggacaccaccgagagccccagcaccatctgggtgtacacccccagccagaaggccatcggcagccggttcgcccagggccggcccatgaacaacgacggcagcttcaccatcaccatgaaggccccccccttcgagcagggcaaggacttcgtggtgctgaccaccaaggcccacggcgtgggcaagaccgaccacagcgacgacacccggacccccgtgacctaccgggaggccacccccgcccccaccggccccaagacccccatcgcccccagcaagcagcccagcaagcaggccgcccccagcaagcaggtgaagcccagcaagcaggccggccccaacaagcagagcaccaccccccagcagaagaccgccgagcaccggagccagacccccgccgcccaccggaccatgaccaagcaggtgagcaccatcggcgccagcaaggtgaccagcggcagcctgacctggggcatccggaccagcttcaccagctacctgcggggccccatcgccaacggcagctggaagctgagcggcggcgccaactggaacggcagcgccttcaccttccccctgaccagcggcagcttcgaccccgccaccaagagcggcagcctgaagtacagcggcagcgtgcacatgaccggccaccacggcatcctggacatgaccctggccgagcccagcctgcagatcaagggcagcaccggccacctgtacctggacgtgaagagcagcagcatggacggcaagaagaccaactacggccgggtggacttcgccaccttcggcgtgagcgtgagcggcaacgccgccatcaagggcagccccgtgaagctgaccgccaccggcgccaaggccttcgccggcttctaccgggccggcgagcccatgaaccccctgagcaccaacctgaccctgagcgccgagaaggtgagccacaacgtgaccgtggacgccgtgaccggcaaggtgatcggcgacgacagcggcaagggcgccggccggggcctgcccgtgacc

SEQ ID NO: 71 - DNA, 918 bp, Propionibacterium acnes; identical to SEQ ID NO: 68

SEQ ID NO: 72 - DNA, 1200 bp, Propionibacterium acnes; identical to SEQ ID NO: 70

SEQ ID NO: 73 - DNA, 918 bp, Propionibacterium acnes
atgagcagcaaccggccccgctctgtggcccaggccgccatcgccacagatggcaagggcatcatcgacaaggacagcagagatgctgtgatcaatgatgccaagctgcgggctgccatcgccggggccctggtgaaggccggcttcagctctgctgatgctgtggccctggccccccgcatcgccaaggagatggccaaggagggcgtgctgctcatcaaccaccacaagctgaaggccctgattggggcccagctgggcctgctgacagatgccaagatccagcgggctgctgctgctgtggacctgggcatcaaggccaccctggccgccaccatcatccccaatgccctgggctctgctgccttcaagaatgccgtcatcgccaacctggtggccgccggcatcgacaagcacctggcccgggccacagctgtggccatcgtggccaccgccctgaaccctgccctgggccccatcgccaagtttgagctgatcaaggccgagattgctgcccaggccgccctgctcatccgccggggcgtgcacctgcagaaggctgccatcgagcacgtgattggccgggcctttgatgctgctgtggccaccgccatcatcagcagccccatcctgtctgcccgcatcgtgacccacctggtgcgggccggcatcgacaagagcatcgccatcagcctggccccccacatcgtgaagcggctggccaaggagcccctgctggccttcaacacagccaagctgatgaagaacatcacccggcagatcgtggatgtgatcaccgctgacaaggccatcaagacagctgagcagctggagaaggagctgcctgccctggatgacctggtgaagaaggccagcagcccccccaagcccacccccacccccacccccacccccacc

SEQ ID NO: 74 - DNA, 1200 bp, Propionibacterium acnes
atggccggccccactgtgactgtcatccctgtgggccgggagggcggggacatcaccatcagcggcaagggcttcagcaccaccggctttggcgtgtatgtggctgtggccccagccagcgtgcctgagttctacggcaacagtgacaagttctacggctatgaccccagcaaggacaccaccgagagccccagcaccatctgggtgtacacccccagccagaaggccatcggcagccgctttgcccagggccggcccatgaacaatgatggctccttcaccatcaccatgaaggcccccccatttgagcagggcaaggacttcgtggtgctgaccaccaaggcccacggcgtgggcaagacagaccacagtgatgacacccgcacccctgtgacctaccgggaggccaccccagcccccactggccccaagacccccatcgcccccagcaagcagcccagcaagcaggccgcccccagcaagcaggtgaagcccagcaagcaggccggccccaacaagcagagcaccaccccccagcagaagacagctgagcaccgcagccagaccccagctgcccaccgcaccatgaccaagcaggtgtccaccatcggggccagcaaggtgaccagcggcagcctgacctggggcatccgcacctccttcaccagctacctgcggggccccatcgccaacggctcctggaagctgtctggaggggccaactggaatggcagcgccttcaccttccccctgaccagcggcagctttgacccagccaccaagagcggcagcctgaagtacagcggctctgtgcacatgactggccaccacggcatcctggacatgaccctggccgagcccagcctgcagatcaagggcagcaccggccacctgtacctggatgtgaagagcagctccatggatggcaagaagaccaactacggccgggtggactttgccaccttcggggtgtctgtgtctggaaatgccgccatcaagggcagccctgtgaagctgacagccaccggggccaaggcctttgctggcttctacagagctggggagcccatgaaccccctgagcaccaacctgaccctgtctgctgagaaggtgtcccacaacgtgactgtggatgctgtgactggcaaggtgattggagatgattctggcaagggggccggccggggcctgcctgtgacc

SEQ ID NO: 75 - DNA, 918 bp, Propionibacterium acnes
atgagctccaaccgcccccgctccgtggcccaggccgccatcgccacagatggcaagggcatcatcgacaaggacagcagagatgctgtgatcaatgatgccaagctgcgggctgccatcgccggggccctggtgaaggccggcttcagctctgctgatgctgtggccctggccccccgcatcgccaaggagatggccaaggagggcgtgctgctcatcaaccaccacaagctgaaggccctgattggggcccagctgggcctgctgacagatgccaagatccagcgggctgctgctgctgtggacctgggcatcaaggccaccctggccgccaccatcatccccaatgccctgggctctgctgccttcaagaatgccgtcatcgccaacctggtggccgccggcatcgacaagcacctggcccgggccacagctgtggccatcgtggccaccgccctgaaccctgccctgggccccatcgccaagtttgagctgatcaaggccgagattgctgcccaggccgccctgctcatccgccggggcgtgcacctgcagaaggctgccatcgagcacgtgattggccgggcctttgatgctgctgtggccaccgccatcatcagcagccccatcctgtctgcccgcatcgtgacccacctggtgcgggccggcatcgacaagagcatcgccatcagcctggccccccacatcgtgaagcggctggccaaggagcccctgctggccttcaacacagccaagctgatgaagaacatcacccggcagatcgtggatgtgatcaccgctgacaaggccatcaagacagctgagcagctggagaaggagctgcctgccctggatgacctggtgaagaaggccagcagcccccccaagcccacccccacccccacccccacccccacc

SEQ ID NO: 76 - DNA, 1199 bp, Propionibacterium acnes
atggccgggcccaccgtgaccgtgatcccagtgggccgggagggcggcgacatcaccatcagcggcaagggcttcagcaccaccggctttggcgtgtatgtggctgtggccccagccagcgtgcctgagttctacggcaacagtgacaagttctacggctatgaccccagcaaggacaccaccgagagccccagcaccatctgggtgtacacccccagccagaaggccatcggcagccgctttgcccagggccggcccatgaacaatgatggctccttcaccatcaccatgaaggcccccccatttgagcagggcaaggacttcgtggtgctgaccaccaaggcccacggcgtgggcaagacagaccacagtgatgacacccgcacccctgtgacctaccgggaggccaccccagcccccactggccccaagacccccatcgcccccagcaagcagcccagcaagcaggccgcccccagcaagcaggtgaagcccagcaagcaggccggccccaacaagcagagcaccaccccccagcagaagacagctgagcaccgcagccagaccccagctgcccaccgcaccatgaccaagcaggtgtccaccatcggggccagcaaggtgaccagcggcagcctgacctggggcatccgcacctccttcaccagctacctgcggggccccatcgccaacggctcctggaagctgtctggaggggccaactggaatggcagcgccttcaccttccccctgaccagcggcagctttgacccagccaccaagagcggcagcctgaagtacagcggctctgtgcacatgactggccaccacggcatcctggacatgaccctggccgagcccagcctgcagatcaagggcagcaccggccacctgtacctggatgtgaagagcagctccatggatggcaagaagaccaactacggccgggtggactttgccaccttcggggtgtctgtgtctggaaatgccgccatcaagggcagccctgtgaagctgacagccaccggggccaaggcctttgctggcttctacagagctggggagcccatgaaccccctgagcaccaacctgaccctgtctgctgagaaggtgtcccacaacgtgactgtggatgctgtgactggcaaggtgattggagatgattctggcaagggggccggccggggcctgcctgtgac

(19) United States
(12) Patent Application Publication
(10) Pub. No.: US 2024/0139302 A1
(43) Pub. Date: May 2, 2024

(54) PROPIONIBACTERIUM ACNES PROPHYLACTIC AND THERAPEUTIC IMMUNE TREATMENT

(71) Applicant: Origimm Biotechnology GmbH, Wien (AT)

(72) Inventors: Sanja Selak, Wein (AT); Christine Triska, Korneuburg (AT); Manfired Schuster, Schrick (AT); Johannes Söllner, Wien (AT); Bernhard Roppenser, Wien (AT); Theresa Weinhäupl, Wien (AT); Max Rössler, Wien (AT)

(21) Appl. No.: 17/801,099

(22) PCT Filed: Feb. 22, 2021
(86) PCT No.: PCT/EP2021/054346

§ 371 Date: Aug. 19, 2022

(30) Foreign Application Priority Data
Feb. 21, 2020 (EP) 20158656.7
Feb. 21, 2020 (EP) 20158659.1
Feb. 21, 2020 (EP) 20158661.7
Feb. 21, 2020 (EP) 20158662.5

(51) Int. Cl. A61K 39/05; A61P 17/10; A61P 31/04; C07K 14/195

(52) U.S. Cl. CPC A61K 39/05; A61P 17/10; A61P 31/04; C07K 14/195

(57) ABSTRACT

The present invention discloses a vaccine comprising one or more of Dermatan sulfate-binding adhesin 1 of P. acnes (DsA1 polypeptide), Dermatan sulfate-binding adhesin 2 of P. acnes (DsA2 polypeptide), and putative iron-transport protein (PITP) polypeptide of P. acnes, and/or a fragment and/or derivative of DsA1 and/or DsA2 and/or PITP, wherein the DsA1 polypeptide and the DsA2 polypeptide comprise from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Thr repeat containing region (“PT repeat region”), and a C-terminal region (“CTR”), and wherein the PITP polypeptide comprises from N- to C-terminus an extended neocarzinostatin family domain (“ENFD”), a first swapping region (“SR1”), a heme-binding domain (“HbD”), a second swapping region (“SR2”) including the C-terminal LPXTG motif, and a hydrophobic C-terminal region (“hLAR”).

The present invention refers to vaccine antigens to be used for prophylactic and therapeutic treatment of P. acnes-associated pathologies.

### 1 BACKGROUND

The gram-positive bacterium Propionibacterium acnes (P. acnes; recently proposed to be re-named Cutibacterium acnes), is a skin commensal predominantly residing within sebaceous follicles which provide a unique lipid-rich environment due to secretion of sebum. Although P. acnes is aerotolerant, it prefers anaerobic growth conditions and uses sebum, cellular debris and metabolic byproducts from the surrounding skin tissue as primary sources of energy and nutrients. Apart from skin, P. acnes has also been found in the conjunctiva, respiratory tract, genitourinary tract and gastrointestinal tract of humans and other animals.

P. acnes is most known for its role in skin disorders such as acne vulgaris. Acne is a disease of pilosebaceous units in the skin, affecting more than 85% of adolescents and more than 20% of population continues to experience symptoms well beyond the teenage period. Acne vulgaris manifests in different severity grades: mild, moderate and severe. Moderate and severe acne account for more than one third of all cases and require medical treatment. Acne can appear also after the puberty, as an adult-onset condition, often associated with hormonal fluctuations which are more prevalent in women. Although the association with acne vulgaris has been most firmly established, it is suspected that P. acnes plays a role also in other severe types of acne, such as acne conglobata, acne fulminans and cystic acne. In addition to dermatological pathology, P. acnes has also been found in corneal ulcers, and is a common cause of chronic endophthalmitis following cataract surgery. Various other inflammatory diseases have been associated with P. acnes, including postoperative prosthetic implant- and device-related infections (implant-associated infections), endocarditis, sarcoidosis, osteomyelitis, allergic alveolitis, pulmonary angitis, the SAPHO syndrome (synovitis, acne, pustulosis, hyperostosis, osteitis) and inflammation of lumbar nerve roots leading to sciatica. More recent studies suggest potential pathogenetic role of P. acnes also in non-infectious diseases such as prostatic cancer, due to its ability to persist intracellularly which may lead to altered gene expression.

Antibiotics have been in use for several decades as one of the most common treatments for acne, both topical and systemic. However, they are not specifically acting against P. acnes and cannot be given long term, especially since the widespread use of antibiotics leads to the rise of antibiotic-resistant bacteria. Vitamin A derivatives (retinoids) represent the second widely used treatment option since these drugs suppress sebaceous gland activity and so indirectly reduce inflammatory lesions. However, oral retinoids are not effective in all patients, and are not a curative drug since recurrence after discontinuation of the treatment is frequent; moreover, it has been associated with severe side effects including elevated serum triglyceride levels, acute pancreatitis, hepatotoxicity, clinical depression, and severe birth defects in pregnant women. Other conventional acne treatments include oral contraceptives and topical bactericidal agents such as benzoyl peroxide. Like retinoids, none of these treatments are long-term effective and curative, they are not suitable for all patients and a prolonged use is also associated with unwanted side effects. Moreover, the drugs currently tested in clinical trials are not very much different from those discovered several decades ago and have shown disappointing results (Zouboulis et al. 2017). Therefore, there is a need for more effective acne therapeutic treatments and with the improved safety profile. Moreover, a prophylactic therapy for acne does not exist, although it would be extremely beneficial especially for the teens with a family history of acne, which is one of the important predisposing factors. Prophylactic therapy in the form of a vaccine specifically designed to induce higher level of protective immunity against P. acnes would also significantly reduce the risk of the implant-associated infections and other pathological conditions in which P. acnes plays a pathogenic role. Despite a high medical need and several decades of research, progress in development of an immune-based therapy against P. acnes-associated pathologies has been very slow and hampered by the confusion in the field regarding the disease pathogenesis and the role of the human immune system in the disease.

The way and the extent to which P. acnes contributes to the pathogenesis of acne and other pathologies, is still debated due to multifactorial nature of the disease and the fact that P. acnes colonizes all individuals, so Koch disease postulates cannot be applied and its identification in different clinical samples has been often attributed to the contamination during sample processing by lab personnel. One additional complicating factor is absence of the predictive and validated animal models that accurately represent the complexity and organization of human tissue and recapitulate human host interactions with skin-colonizing microbes, such as P. acnes. Animal skin is histologically, biochemically and immunologically different from human skin; P. acnes does not colonize animal skin and does not induce acne lesions. Although several different P. acnes animal models have been published by different research groups, these have not been accepted by the wider acne research community as they lack important symptoms of human disease (O'Neill and Gallo 2018). For all these reasons, research efforts in the field have been concentrated on evaluating P. acnes growth and behavior in various in vitro systems, examination of acne-affected skin sections by various histological methods and recently significant attention has been devoted to the analysis of the microbial community that colonizes human skin, collectively known as human skin microbiome, and its interactions with the host cells leading to inflammation or infection are still the subject of intense research effort.

Accordingly, the state of the art at present in the treatment of acne is summarised in Table III of the “Guidelines of care for the management of acne vulgaris” by Zaenglein et al. 2016:

TABLE III
Strength of recommendations for the management
and treatment of acne vulgaris

| | Strength of | Level of |
|---|---|---|
| Recommendation | recommendation | evidence |
| Grading/classification system | B | II, III |
| Microbiologic testing | B | II, III |
| Endocrinologic testing | B | I, II |
| Topical therapies | | |
| Benzoyl peroxide | A | I, II |
| Topical antibiotics (eg, | A | I, II |
| clindamycin and erythromycin) | | |
| Combination of topical antibiotics | A | I |
| and benzoyl peroxide | | |
| Topical retinoids (eg, tretinoin, | A | I, II |
| adapalene, and tazarotene) | | |
| Combination of topical retinoids | A | I, II |
| and benzoyl peroxide/topical antibiotic | | |
| Azelaic acid | A | I |
| Dapsone | A | I, II |
| Salicylic acid | B | II |
| Systemic antibiotics | | |
| Tetracyclines (eg, tetracycline, | A | I, II |
| doxycycline, and minocycline) | | |
| Macrolides (eg, azithromycin | A | I |
| and erythromycin) | | |
| Trimethoprim (with or | B | II |
| without sulfamethoxazole) | | |
| Limiting treatment duration and | A | I, II |
| concomitant/maintenance topical therapy | | |
| Hormonal agents | | |
| Combined oral contraceptives | A | I |
| Spironolactone | B | II, III |
| Flutamide | C | III |
| Oral corticosteroids | B | II |
| Isotretinoin | | |
| Conventional dosing | A | I, II |
| Low-dose treatment for moderate acne | A | I, II |
| Monitoring | B | II |
| iPLEDGE and contraception | A | II |
| Miscellaneous therapies | | |
| and physical modalities | | |
| Chemical peels | B | II, III |
| Intralesional steroids | C | III |
| Complementary and alternative therapies | B | II |
| (eg, tea tree oil, herbal, and biofeedback) | | |
| Role of diet in acne | | |
| Effect of glycemic index | B | II |
| Dairy consumption | B | II |

In this guideline table, all available evidence for the treatment of acne was evaluated using a unified system called the Strength of Recommendation Taxonomy (SORT) developed by editors of the US family medicine and primary care journals. Evidence was graded using a 3-point scale based on the quality of methodology (e.g, randomized control trial, case control, prospective/retrospective cohort, case series, etc) and the overall focus of the study (ie, diagnosis, treatment/prevention/screening, or prognosis) as follows:

I. Good-quality patient-oriented evidence (ie, evidence measuring outcomes that matter to patients: morbidity, mortality, symptom improvement, cost reduction, and quality of life).

II. Limited-quality patient-oriented evidence.

III. Other evidence, including consensus guide-lines, opinion, case studies, or disease-oriented evidence (ie, evidence measuring intermediate, physiologic, or surrogate endpoints that may or may not reflect improvements in patient outcomes).

Clinical recommendations were developed on the best available evidence tabled in the guideline. The strength of recommendation was ranked as follows:

A. Recommendation based on consistent and good-quality patient-oriented evidence.

B. Recommendation based on inconsistent or limited-quality patient-oriented evidence.

C. Recommendation based on consensus, opinion, case studies, or disease-oriented evidence.

It is remarkable that—although most of the therapies aim at killing or reducing P. acnes—no vaccination approach has made its way to the clinic.

Also, the most recent review articles in the present field containing present knowledge and outlook for promising future therapies are either completely silent on vaccination (Lee et al. 2019), are very sceptical about vaccination as a feasible approach (Zouboulis et al. 2017) or do not provide concrete solutions in terms of vaccine antigen selection and composition, but only give an overview of different P. acnes virulence factors involved in pathogenesis and suggest that better and larger studies are needed to even define the proper role of these virulence factors in P. acnes pathogenesis (O'Neill and Gallo 2018; McLaughlin et al. 2019). Remarkably, Zouboulis et al. conclude in their review of 2017 that the “potential for vaccination against P. acnes has been investigated” (Zouboulis et al. 2017, page 819), “but relevant studies stopped in 2011”. It has not clearly been shown that vaccines against P. acnes antigenic structures are effective in humans with acne”. In view of the results obtained for such vaccination approaches, the authors questioned the “potential role of vaccination” for combatting acne in principle “with regard in particular to patient selection, the role of P. acnes in acne, and efficacy of vaccination in diseases of no viral background”.

### 2 OBJECTS OF THE INVENTION

It is an object of the present invention to overcome the deficiencies of prior art suggestions for P. acnes vaccines and to provide improved means and methods to prevent and combat P. acnes infections and P. acnes associated disorders.

It is a further object to provide improved P. acnes vaccines and immunotherapies against P. acnes.

More specifically, it is another object of the present invention to provide vaccines which are protective against a broad range of different P. acnes strains, phylotypes and variants which are able to cause disease, especially vaccines which are protective against at least two or more ribotypes of P. acnes or at least two or more CC types of P. acnes or at least two or more MLST phylotypes of P. acnes or against all three major phylotypes of P. acnes, i.e. Types I. II and III.

Preferably, the present invention may provide protection against P. acnes strains in various states and levels of antigen expression on the surface of P. acnes and particularly against the strains expressing genes which are mediating host invasion and infection (virulence and pathogenesis-associated variants) and whose expression products are able to induce antibodies with opsonizing and killing or neutralizing activity which can increase the efficiency of the host adaptive immune response against the bacterium.

A further objective of the invention is to select the antigens and antigenic epitopes that are immunogenic, accessible to antibody binding on the surface of P. acnes and which induce antibodies with functional activity against P. acnes, such as opsonophagocytic killing, neutralization of virulence potential (e.g. reduce the potential for adhesion to host tissues and cellular invasion, reduce intracellular survival/persistence, reduce fitness of the bacterium via interference with iron acquisition and growth, reduce the potential for formation of bacterial biofilms or prevent the spread of infection by activating adaptive immune response, etc.)

As a further preferred object, the present invention may provide improved antigens with increased immunogenicity and which induce cross-binding and/or cross-reactive antibodies, especially cross-type-reactive antibodies.

Furthermore, it is an object of the present invention to provide immunorelevant polypeptides and vaccines which upon immunization induce antibodies which significantly increase the ability of the phagocytic cells against the bacterium, to prevent their proliferation and virulent behaviour.

Furthermore, another preferred object is that the invention may provide a vaccine composition and formulation with increased stability, purity and amenability for the vaccine manufacturing and administration to the human host.

Furthermore, another preferred object of the present invention is to select antigens that drive generation of antibodies in human host during infection which are able to specifically bind to P. acnes in virulent state and instruct phagocytes to take them up and eliminate them, to reduce their numbers at the infected sites and prevent spread of the infection to surrounding tissues.

Furthermore, it is another preferred object of the present invention to induce immune response against the immunologically relevant antigens that are expressed and accessible on the surface of P. acnes biofilms formed by the strains of different genetic background and which can be specifically bound by antibodies, so to direct immune response towards the bacterial biofilms as well and help prevent their forming and spreading, and/or help their degradation and removal by the immune effectors to enable control of P. acnes in a patient.

### 3 SUMMARY OF THE INVENTION

Therefore, the present invention provides the subject matter claimed and as further described herein, including its embodiments. The present invention provides a practically and clinically relevant strategy for immunologically targeting P. acnes and the therapeutic consequences connected with this microorganism. The present invention aims at immunologically addressing the (human) patient's immune system so as to enable effective prophylaxis or therapy of P. acnes indications. With the products, especially the vaccines and therapeutic and prophylactic methods, according to the present invention, P. acnes indications are effectively controlled, ameliorated or cured. In contrast to the present situation in the field of treatment of P. acnes indications (especially for acne vulgaris: Zaenglein et al. 2016), where no viable vaccination approach has been suggested and immunopathology of acne is indicated as one of the gaps in the knowledge of disease pathogenesis, the present invention provides a suitable and relevant prophylaxis and treatment approach for such disorders. In addition, the drugs currently tested in clinical trials are not very much different from those discovered several decades ago and have shown disappointing results, moreover vaccine strategies based on P. acnes antigens are questioned in terms of feasibility (Zouboulis et al. 2017). The present invention therefore—for the first time—opens up a viable vaccination approach P. acnes indications.

Preferably, the vaccine comprising the innovative polypeptides according to the present invention (which may be used as therapeutic or prophylactic treatment) is protective against a wide range of genetically different P. acnes strains which colonize human host and which are capable of becoming pathogenic in a specific environment or conditions that favor the expression of virulent genes and traits. Preferably, with the present invention, epitopes are identified and characterized as being directly accessible to antibodies when presented by the live P. acnes bacterium and the specific binding of these epitopes by the immune system leads to reduction of bacterial numbers, fitness, growth, virulence or a combination thereof. Preferably, these epitopes characterized in the course of the present invention are presented by the P. acnes bacterium so that they are recognized as surface-accessible epitopes and specifically bound by human serum immunoglobulins raised against P. acnes. Preferably, as has also been observed in the course of the present invention for the purpose of experimental proof, these epitopes are also specifically bound by an animal serum antibody raised against the protein itself, or by P. acnes.

According to a specific aspect of the present invention, the vaccine antigens characterized according to the present invention induce antibodies that lead to a substantial increase in the specific binding and opsonophagocytic killing of at least two P. acnes MLST phylotypes, and/or at least two different CC types of P. acnes and/or at least two ribotypes of P. acnes, as determined by a surface binding measurement employing flow cytometry and in a bactericidal killing assay using immune serum raised against such vaccine antigen.

Preferably, the cross-reactivity, especially cross-type reactivity of antibodies induced after immunization with the products of the present invention can be determined by specifically binding and inducing opsonophagocytic killing activity of at least two or three genetically different types of P. acnes strains. According to a specific embodiment of the present invention, the cross-reactivity/cross-type-reactivity is against at least one Type I and at least one Type II or III strains. According to a further embodiment of the present invention, the cross-reactivity/cross-type-reactivity is against at least one Type II and at least one Type I or III strains. According to a further embodiment of the present invention, the cross-reactivity/cross-type-reactivity is against at least one Type III and at least one Type I or II strains. According to a further embodiment of the present invention, the cross-reactivity/cross-type-reactivity is against at least one Type I, at least one Type II, and at least one Type III strain.

Preferably, the cross-reactivity/cross-type-reactivity of antibodies induced by immunization with a vaccine of the present invention is against two or more of P. acnes strains selected from the group consisting of Type IAI1, Type IA2. Type IB, Type IC, Type II and Type III strains, or Type I-Ia, I-Ib, I-2 and II and III, as defined according to the MLST typing schemes (Lomholt and Kilian 2010; McDowell et al. 2012; Barnard et al. 2015; O'Neill and Gallo 2018: McLaughlin et al. 2019). Even more preferred, the cross-type-reactivity of antibodies induced by immunization with a vaccine of the present invention is against two or more different P. acnes strains, each of these two or more different strains being selected from different Types of the group consisting of Type IA1, Type IA2, Type IB, Type IC, Type II and Type III strains, or Type 1-Ia, I-Ib, I-2, II and III, as defined according to the MLST typing schemes.

Preferably, the cross-reactivity/cross-type reactivity of antibodies induced by immunization with a vaccine of the present invention is against two or more of P. acnes strains selected from the group of various ribotypes, e.g. as determined according to 16S ribosomal sequence differences (Fitz-Gibbon et al. 2013; Tomida et al. 2013).

Preferably, the cross-reactivity/cross-type-reactivity of antibodies induced by immunization with a vaccine of the present invention is against two or more phylotypes determined based on the analysis of a single locus (SLST) as described by Scholz et al. 2014, comprising more than 140 SLS-Types A1-L10, which are documented in an online SLST Database: http://medback.dk/slst/pacnes (Updated: 14 Sep. 2019; Number of STs: 142).

Preferably, the cross-reactivity/cross-type-reactivity of antibodies induced by immunization with a vaccine of the present invention is against at least two different phylogenetic types, more preferably three, even more preferably four, five or six phylogenetic types represented by any of the strains: NCTC737, KPA171202 (DSMZ, German Collection of Microorganisms and Cell Cultures, Braunschweig, Germany), SK137, HL005PA1, HL005PA4, HL013PA1, HL030PA1, HL043PA1, HL053PA1, HL053PA2, HL050PA1, HL050PA2, HL060PA1, HL110PA4 (BEI, Biodefense and Emerging Infections Research Resources Repository, Manassas, VA), P.acn31, PV66 and Asn12 (McDowell et al. 2012), Hung. #2 (Institute of Biochemistry, Biological Research Centre of the Hungarian Academy of Sciences. Szeged) and IAI 008, IAI031, IAI034, IAI035, IAI038, IAI040, IAI042, IAI045, LAI041 (Charite Berlin, Pro-Implant foundation).

Preferably, the vaccine as described herein is protective against development of a pathological condition induced by P. acnes invasion, infection or it can prevent or reduce development of a P. acnes-associated indication. Protection can be determined by at least two or more of the following tests:
a) the induction of antigen-specific antibodies that bind the surface of P. acnes; and/or
b) the functional inhibition of antibacterial activity of other cognate or antigen-specific antibodies against P. acnes; and/or
c) the induction of opsonophagocytic killing activity against P. acnes
d) the prevention, inhibition or reduction of biofilm formation by P. acnes
e) the prevention of implant or a medical device infection by P. acnes
f) the reduction of inflammation or tissue damage caused by P. acnes infection
g) the reduction of capacity for adhesion to human cells, extracellular matrix components or tissues
h) the reduction of cellular invasion and intracellular survival capacity
i) the reduction of capacity to evade or compete with human immune response (e.g. by inhibiting function or expression of virulent factors)
j) the reduction in the capacity to uptake nutrients and minerals required for niche adaptation and survival
k) the reduction of the P. acnes-induced tissue inflammation
l) the increased rate of tissue healing and reparation, following the injury by P. acnes

Preferably, the vaccine formulation is repeatedly administered to a human being, preferably by at least 2 or 3 administrations, or at least by 4, 5, or even more repeated administrations. Preferably, the vaccine can be used in one or more treatment cycles, each involving at least two, or three consecutive administrations within e.g. a period of 1 year in intervals of at least 1 or 2 weeks. Preferably, the treatment cycles may be repeated at least any of 1×, 2×, 3×, or 4×, even more times, e.g. within a period of 5 years, or less.

According to a specific aspect, the vaccine is formulated for administration to a human subject, e.g. a child, an adolescent or adult subject. Typically, the vaccine is provided in a formulation which is suitable for use in a treatment regimen involving both, a prime and boost immunization, preferably wherein the same formulation is suitable for the prime and boost administration.

Preferably, the formulation consists or comprises of one or more antigens or epitopes, as effective ingredients, formulated with an adjuvant.

Preferably, the adjuvant is selected from the group consisting of mineral salts, oil-in-water emulsions, liposomes, TLR agonists, Monophosphoryl Lipid A, saponins, phospholipids, or combinations thereof.

Preferably, the vaccine is formulated suitable for intradermal, subcutaneous (s.c.), parenteral, e.g. intramuscular (i.m.), mucosal, transcutaneous or topical administration.

Preferably, different types of formulations can be used for treating the same human subject, e.g. starting with a systemic treatment or injection, followed by a long-term treatment by local or topical administration. e.g. by a (repeated) application of a vaccine patch.

Preferably, the vaccine comprises 0.1 μg to 5 mg, preferably 0.5 to 1000 μg, more preferred 1 to 500 μg, even more preferred 5 to 300 μg, especially 10 to 200 μg, of each antigen per dose.

Preferably, the antigen is provided as antigen encoding DNA or RNA. Therefore, the invention further provides for a human vaccine comprising a DNA or RNA encoding the vaccine antigen as described herein, preferably as mRNA vaccine with an mRNA molecule with the following structure: 5′UTR-signal peptide-encoded antigen or epitope-3′UTR. In such case, the RNA/DNA vaccine dose can be in the range of 1 μg to 5 mg of DNA or RNA.

Preferably, the dose can be varied when repeatedly administered, e.g. starting with a higher treatment dose, followed by a reduced treatment dose.

In contrast to the practice in the current literature/status in the art, the vaccines and formulations according to the present invention are useable for a wide variety of P. acnes-associated indications and have a general applicability. In the prior art, specific strains that are assumed to cause a specific disease were searched/identified. For example, it was believed that acne is caused by IA1 strains (McLaughlin, 2019), progressive macular hypomelanosis by Type III strains (Barnard, 2016), prostatic cancer promoted more strongly by thiopeptide-producing type IB strains (Sayanjali et al., 2016) or that elevated antibodies have pathogenic, rather than protective role in e.g. sarcoidosis (Schupp et al., 2015). Phylotype independent pathogenicity has been mostly described in the context of implant-associated infections caused by the strains that generate more biofilm, independent of the phylotype (Achermann et al., 2014; Kuehnast et al., 2018). However, the products according to the present invention are useable against the whole variety of P. acnes-associated infections.

According to a specific aspect, the invention provides for a human anti-P. acnes vaccine for use in the treatment of a human subject at risk of or suffering from P. acnes indication, in an effective amount to prevent, treat or ameliorate such disease, preferably selected from the group consisting of acne vulgaris, keratitis, synovitis acne pustulosis hyperostosis osteitis (SAPHO) syndrome, endocarditis, medical implant biofilm infection, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, acne inversa, pulmonary angitis, atherosclerosis, prostatic cancer, progressive macular hypomelanosis and acne conglobata. A specific disease condition treated by the medical use as described herein is any nosocomial infection or inflammation-associated disease where P. acnes plays a role of the causative agent.

This “broad-band” applicability of the vaccines of the present invention also stands in contrast to the outlook and summary of the current research aims in Bruggemann et al., 1019: In this review article where “an overview of the current status on C. acnes research in the postgenomic era” was presented, the authors conclude that there is still a lack of understanding of the underlying disease pathogenesis mechanism and that in order to better understand the disease-causing mechanisms it is crucial to better elucidate bacterial host-interacting properties; which is currently a challenge due to the lack of efficient research tools. The take home message they provide in the end is summarized as follows: “The interactions of C. acnes (and other skin microorganisms) with components of the human immune system and their consequences are currently intensively investigated. These interactions are complex and involve different cell types [ . . . ].” With a view on the future therapy options to tackle, it is pointed to “new therapeutic strategies” that are currently being developed: “In the light of the threat of ineffective antibiotics, as well as the severe side effects of excessive antibiotic therapy and other anti-acne therapeutics, alternative strategies are preferred. Probiotics for the skin are currently being developed [ . . . ].” Remarkably, there is no mention of the vaccines as being one of these “promising future strategies” nor is there any hint in this article, how such P. acnes vaccines could function or how could they be designed.

Preferably, the vaccine is used to induce immunity to prevent and/or ameliorate a P. acnes infection and/or to reduce at least one symptom of such infection and/or to enhance the efficacy of another dose of the vaccine. The vaccine can be used conveniently to prevent, ameliorate, or otherwise treat a P. acnes indication. Upon introduction into a subject, the vaccine is able to provoke an immune response including the production of antibodies and/or cytokines and/or the activation of cytotoxic T cells, B-cells, antigen presenting cells, helper T cells, dendritic cells and/or other cellular responses.

Preferably, the subject is treated for any of prophylaxis or therapy.

Preferably, the subject is a patient with a skin or other affected organ or tissue condition in which the strains from any of Type I, II, or III P. acnes, or a combination of at least two phylotypes of Type I, II, and III, or of at least two ribotypes of P. acnes are involved as pathogenic factors, or a healthy individual that may be susceptible to an infection by any of the P. acnes strains.

Preferably, the invention provides for a method of treating the subject in any of the above indications, or more general any P. acnes indication, wherein an effective amount of the vaccine antigen is administered to the subject.

Preferably, the antigen is administered at least once in an effective amount within the range of 0.1 μg to 5 mg per antigen. Preferably, the vaccine is used for repeated administration to the subject, preferably at least 3 administrations, or at least 4, 5, or even more repeated administrations. Preferably, the vaccine can be used in one or more treatment cycles, each involving at least two, or three consecutive administrations within e.g. a period of 1 year in intervals of at least 1 or 2 weeks. Preferably, the treatment cycles may be repeated at least any of 1×, 2×, 3×, or 4×, even more times, e.g. within a period of 5 years, or less.

According to a further specific aspect, the invention provides for a method of producing a vaccine as described herein, by admixing and/or conjugating antigens as described herein or a DNA or RNA encoding said antigen to a human pharmaceutically acceptable carrier, thereby obtaining a human adjuvant formulation.

The antigen can be also delivered as an antigen-encoding RNA or DNA formulation, preferably as mRNA vaccine with an mRNA molecule with the following structure: 5′UTR-signal peptide-encoded antigen or epitope-3′UTR (or e.g. alternatively also providing a replicase); such formulations have been used for both local and systemic injections in human clinical trials.

### 4 GENERAL DESCRIPTION OF THE INVENTION

The current challenge in the field of P. acnes-associated pathologies is to define a specific prophylactic or immunotherapeutic product in terms of design and detailed description, in terms of how many and which antigens or antigenic epitopes should be included, how they should be combined and produced, to which antigenic epitopes and against which P. acnes strains the immune response should be directed to ensure the desired effect of the immune effector cells against the bacterium and how a product should be manufactured and administered to provide an optimal protective and/or a therapeutic effect in the susceptible host.

The present invention provides a solution to these questions by providing the exact composition of the antigens and antigenic epitopes needed in the vaccine material to induce a protective response against P. acnes, and it also provides the instructions on how the product should be produced and administered. In addition, the invention provides a solution to the question which P. acnes strains should be targeted by immunotherapeutic and prophylactic approaches: these findings also lead to a different conclusion about the most optimal therapeutic strategies and products, making the vaccine with the antigen composition ensuring a much broader strain coverage than currently suggested in the prior art and bringing an additional level of innovation to the current state of knowledge in the field.

Especially, the discovery of vaccines and immune-based therapies has been hampered by the difficulties in studying host immune responses which are regulated by both central and skin immunity, and which can be induced and shaped by both, colonization and infection.

Active vaccination can be based on mono- or multivalent subunit vaccines or whole cell vaccines. In the case of whole cell vaccines, the bacterial cells are inactivated by fixation using various chemical reagents, or killed by heat or cell lysis, and then repeatedly applied orally or subcutaneously. In contrast to subunit vaccines, whole vaccines contain poorly characterized mixtures of many different proteins which are contained within the microbial cell wall or secreted, only some of which may be contributing to protection; however, their efficacy is dependent on the level of native epitope expression and conservation among all different strains with the pathogenic potential. Especially the autovaccines, which are prepared using autologous bacterial strains, add additional level of uncertainty to the quality of the immune response, since the effects of chemical processing on native structures and epitopes can vary greatly among the strains isolated from different individuals and the pre-existing immunity levels to the specific autologous bacterial strain can vary greatly among different patients. For all these reasons the efficiency of the immunization with whole cell vaccines can vary greatly in different subjects and coupled with the increased safety risks, whole cell vaccines are no longer preferred choice of treatment and in many western societies have been banned from use. Nevertheless, the reports of the beneficial effects of autovaccination using autologous bacterial strains of several commensal opportunistic pathogens including P. acnes (Zaluga 1998; LoveckovA and Havlikova 2002) and the recent technological advances allowing rapid sequencing of a large number of bacterial strains have led to a revival of research in the field of prophylaxis and immunotherapy of P. acnes-associated diseases.

Significant efforts have been invested over the last 20 years to identify antigens that could be used as a vaccine. Many putative P. acnes virulence factors have been identified by different research groups and various hypotheses about acne pathogenesis have been proposed. However, since P. acnes expresses and secretes hundreds of different proteins many of which have been proposed to be putative virulent factors, and humans are colonized by a specific signature of different P. acnes strains unique to each person, no specific instruction or evidence has been provided on how a protective prophylactic or therapeutic product should look like in order to make it protective against diseases caused by P. acnes and have the potential to provide therapeutic benefits in a larger percentage of susceptible individuals. Therefore, the present invention, based on the data obtained herein, is able to identify and specify that the current challenge in the field is to define the product description in terms of which antigens would be the best to choose, if one or more antigens would be needed in a vaccine combination, which P. acnes strains should be targeted by a vaccine, which antigenic epitopes the immune response should be directed to and how a product should be produced and administered to provide an optimal protective or a therapeutic immunomodulatory effect to provide protection against a high percentage of strains with virulent potential and reduce the chance of escape mutants, which would be more likely with vaccines containing a single antigen. The status in the field has been described in the most recent publications that reviewed what is known about P. acnes role in acne vulgaris and other pathological conditions and the challenges in terms of discovering a protective therapeutic product are highlighted (O'Neill and Gallo 2018; Bruggemann 2019; McLaughlin et al. 2019). These challenges are currently leading away from a consideration to develop a broadly cross-reactive vaccine against P. acnes-induced infections, but instead designing the product that should act only against one specific phylotype, e.g. in the case of acne vulgaris phylotype IA1 is considered the main pathogenic factor. The difficulties in developing vaccine-based strategies have led to the new directions in the field focusing on the non-immune agents, such as probiotics, light therapy and other approaches that should act to support the growth of the strains considered harmless commensals.

The prior art and most relevant background for the invention that is the subject of this patent application may be summarized as follows:

In 2001 and 2003 two patent applications filed by Corixa Corporation were published which disclosed the results of serological screening of P. acnes phage display library using antibodies and T cells isolated from human donors (WO 2001/81581 A2 and WO 2003/033515 A1). WO 2001/81581 A2 disclosed a series of P. acnes proteins and immunogenic fragments thereof. WO 2003/033515 A1 disclosed antigenic compositions and vaccines against P. acnes. These two applications contained a large number of polypeptide sequences and immunogenic fragments which included all kinds of proteins that could be used as immunotherapeutic or diagnostic products. The candidate antigens which were reactive with human sera were designated as immunogenic P. acnes proteins and summarized in Table 5 of WO 2001/81581 A2. The potential antigen candidates were considered those that based on the bioinformatics analysis and homology to the proteins expressed by other microbial pathogens were predicted to have biological functions that could have relevance in host defense. These included among others, a number of different transferases, enterotoxins, lipoproteins, permeases, proteases, membrane proteins, secreted proteins, adhesins, transporters, hemolysins, penicillin-binding proteins, sialidases, siderophores, zinc-, iron- and manganese-binding proteins (Table 6, WO 2001/81581 A2). Many of the antigens disclosed in this application were later also identified and studied in more detail by other research groups, however only a few have been evaluated in in vitro and in vivo for their suitability as potential prophylactic or therapeutic vaccine candidates (Nakatsuji et al. 2008; Liu et al. 2011; Wang et al. 2018). The most promising antigen candidates included sialidase, lipase and CAMP factor 2, which were protected by U.S. Pat. No. 9,340,769 B2 (filed as US 2011/0243960 A1 and WO 2010/065735 A2).

In 2006, Lodes et al. characterized in more detail four of the immunoreactive P. acnes proteins disclosed in the patent aplications WO 2001/81581 A2 and WO 2003/033515 A1. Two proteins were related to the Corynebacterium diphtheria htaA gene (PA-21693 and PA-4687); the other two (PA-5541 and PA-25957) had some similarity to streptococcal M-like proteins and were found to be similar to each other (68%). Based on bioinformatics analysis the authors predicted PA-21693 to be more conserved among different clinical isolates and its expression was found to be dependent on the iron availability. However, the other three proteins were found to be significantly more variably expressed by different strains due to many frameshifts and mutations identified in the DNA sequence. Moreover, in the strains which were considered capable of expressing the proteins, differences in the cellular location were predicted so that the proteins could be located on the cell surface or secreted. The secretion was suggested to be dependent on the presence of a specific cell-wall binding motif which was termed LP(X)TG domain, in which X could be any amino acid. Bioinformatics and immunoblot analyses led the authors to conclude that the identified proteins had a potential for both phase variation (expression and non-expression) as well as antigenic variation (expression of different antigenic variants of the same protein) among different P. acnes isolates. In addition, the proline-threonine (PT) repeat regions located towards the C-termini of the proteins PA-5541 and PA-25957, were predicted to be highly antigenic. By analyzing the reactivity of human antibodies with the N-terminal protein fragment lacking the PT repeats (NH2-25957) compared to the C-terminal fragment containing the PT repeats (PT-25957), the authors concluded that the antibodies in the acne-negative sera (defined as “having history of mild or no acne”) reacted specifically against the C-terminal fragment containing PT repeats and that this response was of IgG2/3 type, whereas very little or no reactivity was detected against the N-terminal fragment with any of the four tested IgG subclasses. In contrast to these conclusions, the authors found that the antibodies from the acne positive sera (defined as ‘having history of moderate to severe acne’) were directed to the N-terminal part of the protein and that they were of IgG1/4 subtype. The hydrophilic repeat regions identified in the PA-21693 and PA-4687 were also predicted to be highly antigenic, however the reactivity of human antibodies from acne negative and positive subjects against these regions was not studied. The differences in the type of antibodies and the binding of different fragments of PA-25957 and PA-5541 had been suggested as possibly responsible for a deregulated immune response in acne-prone individuals. The authors however provided no instructions whether acne patients can be efficiently vaccinated or how acne patients should be treated in order to induce a more regulated immune response, they also provided no suggestion whether one or more and which of the studied proteins or sequence regions (epitopes) should be included in a vaccine (only the C-terminal PT repeat region was mentioned as antigenic in healthy individuals and potentially relevant for healthy skin). In addition, the expression of the antigens PA-25957. PA-5541 and PA-4687 was suggested to be highly variable, however no instruction was provided how the predicted antigenic and expression variation should be overcome, to ensure the vaccine protectiveness across different P. acnes strains and genotypes that colonize different individuals, which of these strains should be targeted and how, in order to ensure the action against the disease-relevant strains and functional epitopes. All questions related to the specific antigen composition, epitope selection, product design and application were left open and the authors suggested that they should be investigated by future research studies. Accordingly, although the authors conclude from their research that the findings concerning the four proteins PA-21693, PA-4687, PA-5541, and PA-25957 could also be important both in selecting the antigens for a therapeutic vaccine and also in selecting the important epitopes of that antigen that could drive the desired immune response, none of the four proteins has been suggested as a suitable vaccine antigen. Quite on the contrary, the authors concluded that a deregulated immune response against these variably expressed P. acnes antigens could be occurring in individuals with severe inflammatory acne and that this possibility remains to be investigated in the course of such antigen selection for a therapeutic vaccine.

In contrast to these very early attempts to file patent applications purely based on the primary sequences, (rough) bioinformatics, detection of denatured proteins in SDS-PAGE and testing only fours strains (two of Type IA and two of Type II), the strategy according to the present invention was based on real live approaches approaches and quantitative measurements based on actual binding and recognition of specific antigens by the antibodies on the surface of a large number of different P. acnes strains and phylotypes and the ability of antigenspecific antibodies to actually deliver bacterial killing by the human immune system. Accordingly, flow cytometry using antibodies raised against many different antigens was employed and these antibodies were incubated with live P. acnes to test which antigens and which parts of the sequence (peptides and fragments) are exposed on the surface and accessible to antibodies on the surface of the live bacteria; therefore the strategy according to the present invention provided the “real life” evidence that these antigens were accessible to antibody binding on the P. acnes cell surface and that the antibodies are highly cross-(type)-reactive and able to provide the protection against a large percentage of more than 100 tested strains. This is very different from detecting peptides expressed by E. coli bacterium during phage library screening, where any peptide recognized by an antibody will be detected regardless of its actual location and accessibility to the antibodies on the P. acnes live bacterium. In addition, the immune serum raised by these antigens was tested in an antibody-dependent opsonophagocytic killing assay which gives a “real life” assessment of the functional immune consequence of the antibody binding to a particular surface exposed epitope (especially in the human situation, when a vaccine is injected into the patient's skin or muscle, it would be picked up by antigen-presenting cells which should in turn process the antigen and stimulate the T- and B-cells to increase the production of the opsonophagocytic killing antibodies against the epitopes within a specific fragment/sequence region; to help further opsonize and act against the bacteria which breach the skin barrier leading to pathogenesis of disease or infection). Although many antigens were immunogenic (able to raise antibodies when used as a vaccine), there was a significant difference in the ability of these antigens to raise immunologically relevant antibodies that were able to opsonize the bacterium and lead to phagocytic killing of P. acnes.

WO 2011/149099 A1 discloses two short antigenic peptides with 14 and 13 amino acid length termed “PepA” and “PepD” derived from an amino acid sequence of the Genbank/EMBUDDBJ Accession No. YP_056445 and vaccines containing these peptides or nucleotides encoding for these peptides.

In 2010, Holland et al. attempted to identify acne-associated virulent factors by analyzing the proteins secreted by P. acnes during anaerobic cultivation. Many putative virulence-associated factors were identified in this study: the protein PPA1939 was found to be secreted most strongly by all isolates, PPA0816 was secreted by the strains IB, II and 111 and suggested to be a likely surface protein contributing to adhesion and virulence. Other putative adhesins were also identified, e.g. PPA1715, which contained the dipeptide proline-threonine (PT) repeats, as described for PPA2127 and PPA2210 by Lodes et al. (2006) and Holland et al. (2010), however detected only PPA2127 secreted by Type IA strain 266, whereas Type IB strains (KPA171202 and P6) did not express this protein due to mutations and frameshifts within the DNA sequence. Additional pathogenesis-relevant factors were suggested to be PPA2175 (a hypothetical protein, likely endolytic peptidoglycan transglycosylase R1pA) and PPA0687 (CAMP2, a member of the CAMP factor superfamily consisting of five members). The authors suggested that future research should be focused on the more detailed investigation of the secreted virulent factors and their pathogenic significance. According to the authors, a characterization of the function of the secreted factors would require the development of appropriate tools, e.g. a mutagenesis approach to create P. acnes knock-out mutants, and the elucidation of the molecular basis for the observed differences in virulence among different P. acnes clinical isolates. Therefore, although many different proteins were suggested as potentially acting as virulence factors, no specific instruction was given in relation to a specific protein selection or its specific use as a product for either prevention or treatment of P. acnes-associated pathological conditions.

In 2011, McDowell et al. attempted to classify P. acnes strains based on their virulent potential by employing genetic typing and antibody labeling methods. The authors generated two different monoclonal antibodies, one of them, QUBPa1, was found to bind the two antigens that were previously identified by Lodes et al. (2006), PA5541/PPA2210 and PA-25957/PPA2127 (McDowell et al. 2011). By studying labeling patterns of QUBPa1 monoclonal antibody on different P. acnes strains, McDowell et al. demonstrated that the identified antigens were consistently expressed by Type IA strains which were predominantly isolated from acne patients. They suggested that their findings were similar to those of Holland et al., who detected secretion of PPA2127 by Type IA strains, but not by Types II and III (Holland et al. 2010). McDowell et al. (2011) proposed Type IA strains to be associated with pathogenesis of acne and Types IB, II and III to be associated with other types of infections. They have also confirmed the findings of Lodes et al. (2006), that the antigens PA5541/PPA2210 and PA-25957/PPA2127 were prone to phase variation, antigenic variation and secretion and based on their ability to bind dermatan sulfate, the authors named these two proteins dermatan sulfate-adhesins: DsA1 (PA-25957/PPA2127) and DsA2 (PA5541/PPA2210). This study was focused on clinical significance of the strains expressing various putative virulence factors (other virulence factors were also found to be produced by IA strains, e.g. neuraminidase and lipase GehA) and the use of these findings as supportive tools in classification of different P. acnes strains. Apart from being suggested as important virulence factors, the authors did not provide suggestions on the possible prophylactic or therapeutic interventions based on either these or any other described antigens. Monoclonal antibody (MAb) typing by immunofluorescence microscopy (IFM) was carried out as described by McDowell et al., 2005 (McDowell et al., J. Clin. Microbiol. 43 (2005), 326-334). McDowell (2012) produced monoclonal antibodies by immunizing BALB/c mice with killed whole cells of different P. acnes strains. Hybridoma cell lines producing P. acnes-specific MAbs were cloned by limiting dilution (McDowell (2005)). Isolates were examined for their reactivity with mouse monoclonal antibodies QUBPa1 and QUBPa2, which target strains within types IA1 and II, respectively. These monoclonal antibodies were used to label P. acnes strains for IFM and IFM was used for phylogenetic structuring (the identification of type IA and type II strains). A range of genes was examined, especially those encoding cell surface-associated antigens, which can have a strong discriminatory power (McDowell (2011)). They state that their previously used monoclonal antibody QUBPa1 is type IA-specific (p. 10). Also McDowell (2011) used monoclonal antibodies that are specific only for P. acnes strain IA1, QUBPa1, for the purpose of purification on an immuno-affinity column as well as labeling for the immunofluorescence microscopy as indicated on p. 2000: IFM analysis of type I strains with QUBPa1 reveals polar and septal labelling on the cell surface (McDowell et al., 2005). [ . . . ] For type IA strains, IFM with QUBPa1 in fact provided evidence of within-strain phase variation in expression and not the ability to bind the cell surface and induce bactericidal effects against different phylotypes. This monoclonal antibody was thus not suggested for the use of the identification of key immunogenic and immunologically relevant epitopes but for the identification of the nature of the antigen (i.e. the phase variation) as well as a research tool to differentiate between P. acnes genotypes. Besides, the expression of cell-surface adhesins with the capacity for phase/antigenic variation and enhanced immunogenicity, and the production of specific virulence factors that aid overgrowth as well as the degradation of host tissue components was used as a rationale for the association of Type IA strains with acne. However, the present invention aims at providing vaccines which would deliver a broad cross-reactivity (i.e. a cross-type-reactivity, and not being restricted to Type IA1) and therapeutic benefit in combating P. acnes-associated diseases and infections, and not to identify research tools for labeling a particular P. acnes phylotype.

Also McLaughlin (2019) states that the “IFM analysis of skin biopsy samples with monoclonal antibodies (MAbs) has shown the presence of both type IA and type II within the sebaceous follicles of both acne and control subjects [ . . . ]”. Type II strains were detected with a different monoclonal antibody (i.e. QUBPa2 (McDowell et al. (2011))) which did not recognize DsA1/DsA2. McLaughlin (2019) states on p. 15 that “Using a MAb (QUBPa1) which was subsequently revealed to target DsA1 and DsA2 antigens on the cell surface, only strains representing type IA1, IA2 and also IC were immunoreactive displaying both polar and septal labelling. [ . . . ] We can speculate that the potential for type IAs to modulate their interaction with the host immune system via the DsA immunogenic proteins may be important in the recurring nature of acne.” Therefore, McLaughlin (2019) refers with the term “immunoreactive” to a polar and septal labelling of DsA1 and DsA2 by a monoclonal antibody. Furthermore, they “speculate” about the reason for recurring nature of acne and they do not mention an immunotherapy as a conclusion, let alone an indication or motivation how to design a product for treating acne. Moreover, the article of McLaughlin (2019) is speculating about the association of various virulence factors with the pathogenic significance of the acne-associated strains among a long list of antigens summarized in the FIG. 12 and Table 2; and is careful to suggest that only strains of IA1 phylotype should be targeted by a vaccine. There was no suggestion how to make such a vaccine, especially not how to make a vaccine which goes beyond targeting IA1 strains. McLaughlin (2019) revealed the pathogenic potential of the KPA171202 strain compared to other strains based on the genes for putative tissue degrading factors, including hemolysis, co-hemolytic Christie-Atkins-Munch-Peterson (CAMP) factors, sialidases, endoglycoceramidases, lipases, porphyrins and hyaloronate lyase (HYL), as well as genes for cell wall-/cell envelope-associated proteins with inflammatory potential, and proteins that contain multiple prolin-threonin repeats (PTRs). Another approach how to define putative virulence factors was to compare the transcriptome of KPA171202 with the type IA1 strain 266 (pleuropulmonary infection) and the identification of a differential expression of 119 common genes during mid-log phase. These included genes for triacylglycerol lipase endoglycoceramidases, DsA1 and HtaA/P071. However the conclusion why these gene products might represent putative virulence factors was only based on the fact that those genes were upregulated in 266 which gives no clear indication for a vaccine product, the selection of antigens and which ones to use and how to make feasible for application in humans. Moreover, a virulence factor only indicates that the strains expressing such a gene are more virulent, but it gives no indication at all for the suitability of such a factor in a vaccination context. Therefore, also this prior art pointed into a different direction compared to the present invention; quite in contrast, the “vaccine development” section of McLaughlin et al. consequently referred to CAMP factor 2 only as a potential vaccine target.

The expression of different virulence-associated factors was further studied by Brzuszkiewicz et al. (2011), who provided additional insights into the activity of different virulence-associated genes, among many others they also mentioned GehA lipase (PPA2105), polyunsaturated fatty acid isomerase PAI(PPA1039), the three proteins described by Lodes et al. (2006): PA-25957/PPA2127, PA5541/PPA2210 and iron acquisition protein HtaA (PA-4687/PPA0786). Similar to McDowell et al. (2011) and Lodes et al. (2006). Brzuszkiewicz et al. (2011) also hypothesized that strain differences in the virulent potential may be due to the differences in the expression of virulence factors by different phylotypes, and they suggested that besides these, other virulent factors are likely also important. They also suggested that a disease-causing potential of different P. acnes strains is not only determined by the phylotype-specific genome content but also by variable gene expression. The authors provided a potential scenario about how an inflammatory acne lesion may develop, which strains might be important but did not suggest any specific instruction about the selection of vaccine material and design of a product for either disease treatment or prevention. Like others, they suggested that future research efforts should be invested to elucidate factors responsible for differential behavior of the strains and the underlying mechanisms responsible for the P. acnes-induced inflammation. They postulated that a host with an unbalanced immunological response to P. acnes may be vulnerable to the presence of the strains with a much higher virulence potential and that the strains from the phylogenetic group I-Ia, which express higher levels of adhesins and other virulence factors, are more likely to induce pathological symptoms in the predisposed host. Also Mayslich et al. (Microorg. 9 (2021), 303) reviewed the actual status of research on virulence factors and P. acnes infections. It was recognized that P. acnes is—as a commensal bacterium—only weakly immunogenic and therefore in general tolerated on the skin of the host. It is assumed that it is only involved in pathogenic circumstances as an opportunistic pathogen when it expresses different antigenic components at its surface which may lead to a strong inflammatory reaction in the skin and in many other internal organs. Mayslich et al. review the various virulence factors (especially CAMP2) but arrive (in their summary and conclusion of the state of the art) at two remaining “main unresolved questions”: First, whether a particular P. acnes type can become more pathogenic in response to environmental changes in relation to the expression of a specific virulence factor; and second, how the interaction of P. acnes with the skin microbiota influences its pathogenicity. Both questions and, of course, a way to tackle this pathogenic development, remain unanswered. In 2011, Liu et al. published a study in which they used a monoclonal antibody against CAMP 2, one of the antigens identified previously in serological screens by Corixa and later by secretome proteomics by Holland et al. (2010). This protein had been identified also as a virulence factor secreted by other bacteria, with the reported function as a hemolytic factor and pore-forming toxin. The effects of monoclonal antibody against CAMP 2 antigen were suggested to be important for neutralization of the secreted CAMP 2 virulence factor, to attenuate its virulence. The authors specifically suggested that the attenuation of P. acnes virulence by targeting secreted virulence factors should be the goal of acne immunotherapy, rather than targeting the proteins located on P. acnes cell surface and with this new approach they have abandoned the previously chosen vaccine candidate, P. acnes sialidase, which was a cell surface protein. In fact, all recent review authors conclude that new promising therapeutic approaches should focus on the “modulation of the population of C. acnes strains on the skin, without inducing a negative reaction [ . . . ]” aiming at products that are designed so to not directly act against P. acnes and disturb the microbiome. Therefore, introduction of commensal “healthy” strains of P. acnes is regarded as being better than acting against the “pathogenic” strains, as e.g. a vaccine approach would do (see e.g. O'Neill and Gallo 2018; Bruggemann et al., 2019; Cong et al., 2019; Mayslich et al., 2021; Petronelli. 2021). In summary, all these recent reviews suggest that the new therapeutic approaches to address the patholpgy of P. acnes-associated infections by moving away from the vaccine approach (suggested as being promising almost 20 years ago) or raising extreme caution/warning against it (“without inducing a negative reaction”; Mayslich et al., 2021).

In 2014 Bek-Thomsen et al. attempted to study pathological processes involved in the formation of acne by proteomic analysis of the sebaceous follicular casts extracted from healthy and acne-affected individuals (Bek-Thomsen et al. 2014). They looked for proteins that might be differentially expressed in healthy compared to acne-prone skin pores, however they could not identify the evidence for differential expression of any virulence factors but instead concluded that both healthy and acne-affected skin pores contain the same protein composition. The most abundant P. acnes proteins identified in both acne-affected and healthy follicular samples were dermatan sulphate adhesins DsA1 (PA-25957/PPA2127), and DsA2 (PA5541/PPA2210), CAMP factors 1 and 2, and an uncharacterized lipase, PPA1796. Other proteins they considered important were myeloperoxidase, lactotransferrin, neutrophil elastase inhibitor and vimentin. Contrary to McDowell et al. (2011) they concluded that the lipase GehA (glycerol-ester hydrolase A, PPA2105), was likely not an important virulence factor, since it was found in only a minor percentage of heathy skin samples and in none of the acne-affected skin samples. Instead, the authors suggested that a novel, uncharacterized lipase PPA1796, was more important in vivo and they proposed a new gene name for this potential virulence factor, GehB. The most significant biological process in acne was found to be ‘response to a bacterium’, this has been concluded based on the identification of a high level of human host proteins known to be involved in immune responses, tissue remodeling and healing. In addition, the authors concluded that both healthy and acne-affected sebaceous hair follicles are colonized by the same strains which they suggested to be Type IA, based on the high level of expression of the two adhesins DsA1 and DsA2, which were not expressed by Types IB, II and III. In addition, they suggested that there might be some important differences among Type IA strains in their virulent potential and that this should be investigated by future studies. Although this study presented potentially interesting data about the proteomic content of the pilosebaceous hair follicles in acne compared to healthy skin, the authors admitted that the study had many limitations which make it difficult to draw more specific conclusions and they did not provide instruction on how to use this knowledge to develop any specific product for acne treatment. Instead, they postulated that certain virulent strains of P. acnes may exploit the presence of specific human host molecules identified in this study, such as vimentin, to invade skin cells and so induce inflammation and that the future research should be directed to identifying the origin of vimentin expression in the pilosebaceous follicular casts.

In 2015 Achermann et al. searched for potential vaccine candidates by investigating P. acnes proteins that were produced in vivo during a biofilm infection with one specific P. acnes strain and by testing animal sera for the presence of antibodies against the proteins found in P. acnes cell wall- and membrane-associated fractions that were separated by two-dimensional electrophoresis. They identified 23 immunogenic proteins proposed to be potential P. acnes vaccine candidates. Among these, the most promising vaccine candidate was suggested to be glyceraldehyde-3-phosphate dehydrogenase (GADPH) identified by the accession number G7U8Y4, the same protein previously identified by Holland et al. (2010) and Bek-Thomsen et aL. (2014) as PPA0816. An ABC tranporter (D4HAH2) was found of particular importance; malate dehydrogenase (Q6A6Z5), DnaK chaperon (W4TZS5), methylmalonyl-CoA mutase (E4D8Y8) and several other proteins were also mentioned among the best candidates. The detection of the proteins in cell fractions separated under denaturing conditions and analyzed by Western Blot does not provide any evidence that these proteins were exposed on the bacterial cell surface and accessible to antibody binding. The single antigens were also not tested as vaccine material for their ability to induce antigen-specific and functional antibodies neither against the P. acnes strain used in this study or on any other strain from many other known P. acnes genetic types. Whether these antigens were immunogenic in human host and detectable by human antibodies, was not tested either. Nevertheless, the authors proposed that further studies should be performed to find out the potential of these candidates in preventing chronic P. acnes biofilm-mediated infections or for use in diagnostic tests.

In parallel with the efforts to identify factors responsible for the pathogenesis of P. acnes-associated diseases, novel technological developments in the field have introduced more complex studies of the entire populations of microbes on human skin and attempts were made to additionally sub-classify P. acnes strains into more or less virulent and to identify health-associated or true commensal strains.

Phylogenetic studies based on multilocus gene sequencing, as well as whole-genome analyses of isolates from the Human Microbiome Project (HMP) have provided valuable insights into the genetic population structure of P. acnes. The Multi Locus Sequence Typing (MLST) generates different sequences defined as distinct alleles which are then used to generate sequence types (STs) for every P. acnes isolate. Two different MLST typing schemes have been developed which divide P. acnes into closely related clusters IA1, IA2, IB, IC, II, and II (McDowell et al. 2012) or I-1a, I-1b, I-2, II, and III (Lomholt and Kilian 2010).

More recent genomic studies have led to identification of additional sub-divisions based on whole genome sequencing of all known P. acnes strains, which led to a single locus sequence typing (SLST) scheme that is especially helpful in identifying P. acnes ST diversity in mixed microbial communities (Scholz et al. 2014). An alternative single-locus approach based on 16S rRNA profiling has been used to investigate the pathogenic role of P. acnes in acne and other P. acnes-associated pathologies. Specific P. acnes strains have been proposed to be associated with acne and some others to be associated with healthy skin or involved in other types of infections (Fitz-Gibbon et al. 2013; O'Neill and Gallo 2018; McLaughlin et al. 2019).

Although these studies relied mostly on the analysis of the strains isolated from different individuals and had limitations related to skin sampling and analysis methods, they have provided directions for the follow up research studies and influenced current state of the knowledge in the field. These developments have directed research efforts towards the identification and analysis of the proteins expressed by the selected P. acnes genetic types considered virulent or by analyzing the proteins found in different types of clinical material.

Following the novel findings on the significance of specific ribotypes in the pathogenesis of acne. Yu et al. (2015; 2016) employed proteomic analysis to analyze the expression of different proteins by the P. acnes strains that were classified according to a specific ribotype considered to be associated either with acne or with healthy skin. These authors found that the expression of several proteins was variable among different ribotypes and since these ribotypes have been previously associated with disease vs health and induced different amounts of cytokine secretion by human cells, the authors proposed these proteins to be investigated as potential vaccine candidates. The attention was especially drawn to the proteins corresponding to gi 50843388 (PPA1939), gi 50843565 (PA-25957), gi 50843218 (PPA1758) and several other proteins of unknown function. The authors also suggested that the search of vaccine candidates should not be directed to looking for the antigens that induce antibodies, but instead look for those that induce strong Treg cell responses. Therefore, they were leading away from the proposal by Lodes et al. and others, that an antigen candidate should be antigenic and reactive with human antibodies. This direction was suggested based on the assumption that P. acnes is a commensal bacterium inducing inflammation and that an optimal vaccine antigen should act to reduce adaptive immune response activation, rather than enhance it (Yu et al. 2015). In addition, the authors excluded PA-5541 (gi 50843645) from the list of potential vaccine candidates, due to the findings that it was found expressed only by the strains associated with acne or the strains that had a neutral significance and was not expressed by the strains associated with health (II RT6) (Yu et al. 2016). In summary, many of the proteins disclosed in these studies were previously also identified by others and the authors did not provide either a solution or a concrete proposal in terms of the vaccine composition and product design, but only suggested future research directions in the discovery of an optimal vaccine and in studying disease pathogenesis.

Additional publications in 2016 presented research findings related to some of the proteins previously identified by others: PPA1939 (Allhorn et al. 2016) and PA-25957 (Grange et al. 2017). These two proteins were found interesting based on their putative additional biological functions: PPA1939 was proposed to be relevant for heme binding and antioxidant capacity, and PA-25957 was suggested to play a role in fibrinogen binding. In addition, the virulent potential of PA-25957 has been additionally supported based on its interaction with fibrinogen and the expression being restricted to specific disease-associated P. acnes phylotypes. These authors did not propose how this protein could be used as a vaccine product, but only hypothesized about its role as a virulence factor which could in addition to binding dermatan sulfate also act by forming ‘clumps’ inside hair follicles in contact with fibrinogen. They also suggested that future research should focus on developing tools for testing the ability of different protein fragments to bind fibrinogen and that the protein is likely to show significant variability in the level of secretion and expression on the surface of different strains, which should be investigated by future studies in order to gain more understanding about its potential significance.

Recently, CAMP 2 immunotherapeutic product development has shifted from a monoclonal antibody towards a therapeutic vaccine which should act to induce antibodies that reduce P. acnes cytotoxicity and prevent the release of cytokines by keratinocytes and phagocytic cells, thereby acting against inflammation (Wang et al. 2018). Especially negative P. acnes effects on the survival of phagocytes has been presented as an important evidence for CAMP 2 virulence and this has supported the argument for a beneficial effect of the immune response induced by the CAMP 2 vaccine. The limitation of this study is that the effects of CAMP 2 antibodies on the viability of phagocytes in the presence of P. acnes, were not tested and compared in the same assay to the effects of antibodies against other antigen candidates and a larger panel of P. acnes strains, making it difficult to evaluate the significance of these findings.

The confusion in the field regarding the optimal vaccine candidate still continues due to constantly evolving and changing hypotheses about the significance of different P. acnes strains in the acne pathogenesis, the potential role of other microbes that are a part of the human skin microbiome and the unresolved controversy over the functional significance of human immune response against the bacteria. The caution in selecting antigen candidates for various vaccination approaches has been expressed in a recent review in which it was stressed that it is still unclear which P. acnes strains are virulent and should be targeted by vaccines, and that it is still not clear if acne severity is driven by the patient's basal innate immune condition or by the P. acnes virulence level and that this should be considered when designing immunotherapy protocols and especially deciding on the choice of the target antigen (Contassot 2018). The author rationalized that a specific inhibition of secreted virulence factors could limit the risk of unwanted targeting of nonpathogenic bacteria and overcome a possible selection of resistant bacteria. More importantly, the author suggested that vaccines based on surface antigens should be highly specific to avoid ‘off target’ effects and activity against P. acnes strains that may be beneficial to the host. However, if acne severity is driven by the patients' innate immune condition and not by specific virulent strains, this should have consequence on the immunotherapeutic approach and especially on the type of antigen selected for the vaccine.

Although many different molecules and proteins have been studied and proposed as potential virulent factors, only a few of them have been tested as potential vaccine or therapeutic product candidates, and the current status in the field makes it impossible to make a firm conclusion about the potential of any of the described molecules as suitable acne vaccine product candidates. Some virulent factors were found to be secreted, some expressed in the cell membranes but their accessibility on the bacterial cell surface and its functional immunological relevance have not been studied, some were found to be expressed only by specific strains whose virulent potential was not well understood. Some potential virulent factors were suggested to be expressed by specific phylotypes considered virulent, but with a high antigenic diversity and a variable expression, so no specific proposal for their use in a specific product has been suggested or demonstrated as possible. In addition, all studies contained significant limitations that made it impossible to derive firm conclusions about the immunologically relevant superiority of different antigens. For example, the ability of the identified antigens to induce immune responses in a form of a vaccine was not tested or the conclusions were based mainly on the results obtained with a single antigen using suboptimal methods. For example, bioinformatics analysis provides limited information about the likelihood of a particular gene product expression and its cellular location. Although proteomic analysis can provide more valuable information about the actual level of the gene expression in a particular bacterial cell within a defined genetic background, it is also prone to errors and not useful in evaluating the potential of a certain gene product as an immunotherapeutic target. Factors such as stability of the expressed proteins during the biological material preparation, behavior during the SDS-PAGE separation, the easiness of solubilization and extraction from cellular membranes can all affect the outcome of the analysis and lead to suboptimal conclusions. Moreover, proteomic analysis does not provide any information about the accessibility of the protein and its immune protection-relevant epitopes on the bacterial cell surface. In addition, many studies focused on testing only a few selected P. acnes strains; the clinical samples used in analysis were obtained from a few donors and collected in a way that introduced additional variations that made it difficult to derive more reliable conclusions about the suitability of different molecules or their derivatives as product candidates. Coupled with the newest developments in the field that added additional conflicting information about the importance of different factors in mediating P. acnes-induced pathologies or the factors that may provide protection, the skilled person in the art could not guess with confidence based on the current state in the art, which antigens or epitopes and how many of them would be needed to design an optimal vaccine product, if such product should act to induce, reduce or modulate an immune response to a specific protein or its epitope, and how to design it to be protective against all the strains with a pathogenic potential, and which strains should be targeted to achieve the highest benefit for a host.

Systemic immunological studies including many genetically different P. acnes strains and comparing different antigens and fragments to each other using the same immunological, vaccine-relevant methods are lacking in the prior art. The progress has been also hampered by the lack of understanding of the interactions between the host and the bacterium and why only in certain individuals P. acnes is associated with the pathology of disease. This has also made the research community cautious in developing P. acnes-based immunotherapeutic products, since the consensus is still not achieved in terms of which strains of P. acnes should a product act against and some researchers have been very cautious in considering a vaccination approach for the fear of the negative effect of the vaccine on the strains which may be beneficial to human health. This caution is especially due to the limited understanding of the host factors that contribute to the disease, since P. acnes is generally considered to be resistant to phagocytic killing by immune cells (Webster et al. 1985) and has been also reported to be able to escape phagocytic killing, even after being taken up by the phagocytic cells (Fischer et al. 2013). In addition, different intracellular pathways of P. acnes trafficking have been described, one of them involving autophagy, which is a non-specific response not dependent on P. acnes antibodies, it varies depending on the cell type and conditions, and may be occurring in association with innate immune response, which may not lead to the activation of the adaptive immunity. Therefore, either homeostatic or protective, adaptive immune response may be activated depending on how the host immune system perceives the consequence of the bacterial interaction with the host and the amount of danger the bacterium poses to the host.

The progress in the identification of a suitable vaccine has been slow also due to the lack of available methods and models for a vaccine antigen identification. Contrary to a more typical infectious disease, the immune system of all humans has been already primed to respond to P. acnes and the antibodies against this bacterium can be found in the blood of both, patients and healthy individuals. Since immune system can be stimulated by the bacteria not only in the skin, but also at other colonized sites (e.g. oral cavity, gut) and the level of immune response to P. acnes is also influenced by the interplay of individually specific factors which can result in the immune system perceiving the stimulus as more or less ‘dangerous’: just a mere presence of antibodies in the blood of a certain individual is not an indication of their actual clinical effectiveness or significance. Therefore, selection of antigen candidates based on serological screening in the absence of any functional assays is bound by the same limitations as determining the pathogenicity of a specific P. acnes strain by its mere presence and isolation from the skin of different individuals. Without knowing anything about the antbacterial activity of antibodies against P. acnes, it is difficult to discern which antigen-specific antibodies can help the immune system in controlling the growth and invasive behavior of the bacterium and which ones may lead to production of antibodies that are only a side product of persistent colonization, secretion and antigen presentation in the course of a normal cellular turnover.

Therefore, the current challenges in P. acnes immunoprotective antigen identification are multifold and involve both the lack of a suitable disease model and the lack of reliable functional in vitro assays for antigen screening. In other typical vaccine discovery projects, even if animal models of disease are not available, functional in vitro assays can be used to select suitable antigens for a protective vaccine. For example, the discovery of the marketed vaccine against Neisseria meningitidis was made possible by studying the effect of antigens in inducing bactericidal antibodies in animals which were tested in a serum bactericidal assay (Giuliani et al. 2006); the discovery of the vaccine against Streptococcus pneumoniae was facilitated by studying the opsonophagocytic killing effect of antibodies in an in vitro opsonophagocytic killing assay (van Westen et al. 2013), and several anti-viral vaccines have been discovered based on a neutralizing effect of antibodies on the viral replication measured in an antibody neutralization assay (Vanblargan et al. 2016). No such functional in vitro assays have been developed for P. acnes vaccine studies. Moreover, in the current state of prior art P. acnes is considered resistant to phagocytic killing and perpetuating inflammation due to extensive immunostimulation which is result of the expression and secretion of a large number of putative virulence factors. Which of these factors may be helping the host immune cells reduce the bacterial activity and density in the inflamed tissue and thereby have the potential to reduce or resolve the inflammation, is currently a question of much debate and no satisfactory answer has been yet offered, apart from the proposal that in acne vulgaris the strains from phylotype IA1 may be more virulent than others and that an ideal acne vaccine candidate should be searched among those that are expressed by these strains (O'Neill and Gallo 2018; McLaughlin et al. 2019). This leaves the unsatisfactory answer to the significance of the findings that phylotypes other than Type IA1 are frequently isolated from other types of P. acnes-associated infections (e.g. implant infections) and that these phylotypes also colonize human skin which during the surgical procedure can infect susceptible individuals. The possibility that the same strains may be able to cause diseases in different context, depending on the opportunity and the susceptibility of the host, has not been adequately addressed in the prior art. Instead, the focus was on trying to associate specific P. acnes phylogenetic types with specific diseases, but this approach has led to many unsatisfactory or even contradictory findings and lots of debates among different research groups (Fitz-Gibbon et al. 2013; Eady and Layton, 2013; Alexeyev and Zouboulis, 2013).

The most novel concept that recently evolved and influenced researchers in this field is the concept of skin microbiome, and therefore, the most recent studies have focused on the identification of the pathogenic factors that may be the result of the disturbance of the balance in the interactions within a total microbial community colonizing a specific area of the skin acting and the term of the microbiome dysbiosis has been introduced to indicate such a possibility (O'Neill and Gallo 2018). Therefore, the current efforts of the researchers are focused on studying the interplay of different microbial species and developing the products that would address ‘microbiome dysbiosis’. Although this extremely novel concept is lacking the explanation of what “a healthy” microbiome should look like in each individual; a great number of researchers are already developing next generation acne treatment products based on the mixtures of specific bacterial strains or their products as skin probiotics and prebiotics. The most recent reviews on the progress in the field in the search for the therapeutic products still describe many different virulent factors as likely contributing to pathogenesis, however, only a handful of suggested virulence factors have been actually tested as potential vaccine candidates: CAMP 2 and the group of antigens identified by Acherman et al., 2015 (O'Neill and Gallo 2018); however even these antigens were not systematically tested and compared in the same assays and using a larger panel of strains that would allow a non-biased selection of the most suitable candidates (McLaughlin et al. 2019). Therefore, even among these candidates it is difficult to guess, without further tests, which ones, if any, would be most protective and suitable as a vaccine candidate.

Over a period of more than 15 years a great number of different proteins were proposed to be contributing to P. acnes virulence and their biological and potential clinical significance has been studied by many different research groups. Their cellular location, secretion, expression by different bacterial strains and in different environments (in different growth media, under different in vitro growth conditions or in human skin) and in some cases antibodies that recognize these antigens have been detected in the serum of acne patients as well as in healthy individuals. However the significance of these findings could not be extrapolated in order to design a vaccine product for treating acne or other P. acnes-associated pathological conditions. Since many proteins were studied by more than one research group and their biological and pathological significance is still under investigation, in the public databases as of today (e.g. UniProt: https://www.uniprot.org) many of the proteins are still indicated as “putative” or “uncharacterized”, or they are named according to the suggested, hypothetical biological function, e.g. “conserved protein, putative for Fe-transport”. To facilitate understanding the data presented in this invention, the sequences and the nomenclature used in the public sequence databases as of the date of tiling this invention are provided in the summary Table 1.

### 5 DETAILED DESCRIPTION OF THE INVENTION

For the purpose of the present invention, the following names and abbreviations are used for the polypeptides of P. acnes referred to in the present invention (with the annotations in the public databases and in the literature citations):
P002 (SEQ ID NO:1): an example for the CAMP factor, also known as Christie-Atkins-Munch-Petersen (CAMP) factor (Liu et al. 2011; Wang et al. 2018);
P005 (SEQ ID NO:2): an example for the Glyceraldehyde-3-phosphate dehydrogenase; also described by Holland et al. (2010) and suggested among the potential vaccine candidates by Achermann et al. (2015).
P018 (SEQ ID NO:3): an example for the “repressor”;
P022 (SEQ ID NO:4): an example for the “uncharacterized protein”, also described in published literature as “host cell-surface attachment protein PA25957” (Lodes et al. 2006) and “dermatan sulfate-binding adhesin/DsA1” (McDowell et al. 2011),
P027 (SEQ ID NO:8): an example for the “uncharacterized protein”, also known as “host cell-surface attachment protein PA5541” (Lodes et al. 2006) and “dermatan sulfate-binding adhesin/DsA2”;
P028 (SEQ ID NO:13): an example for the “conserved protein, putative for Fe-transport”, also known as PA21693 (Lodes et al. 2006),
P032 (SEQ ID NO:21): an example for the Translation initiation factor IF-2;
P035 (SEQ ID NO:22): an example for the Malate dehydrogenase as identified by Achermann et al. (2015); Bek-Thomsen et al. (2014) and by Yu et al. (2016) as 50843200.
P042 (SEQ ID NO:23): an example for the “TED domain-containing protein”, also identified by Holland et al. (2010) as PPA1715 and by Yu et al. (2016) as gi 50843175.
P046 (SEQ ID NO:24): an example for the “probable endolytic peptidoglycan transglycosylase RIpA”; also identified by Holland et al. (2010) as PPA2175
P068 (SEQ ID NO:25): an example for the “uncharacherized protein” in the protein sequence database—this protein has been also described by Holland et al. 2010 as PPA1939; by Yu et al. (2015) and Yu et al. (2016) as gi 50843388, and as RoxP by Allhorn et al. (2016);
P069 (SEQ ID NO:26): an example for the “outer membrane lipoprotein”, which has been identified by Yu et al. (2015) as gi 50843218.
P070 (SEQ ID NO:27): an example for the “ABC transporter, ATP-binding protein” and suggested as a potential vaccine candidate by Achermann et al. (2015).
P071 (SEQ ID NO:28): an example for the protein described by Lodes as PA-4687, and at the time of this application in public databases this protein is termed “HtaA-like surface protein”; this same name has been also used by other authors who considered this protein among important virulence factors (Brzuszkiewicz et al. 2011; McLaughlin et al. 2019).

In the present invention the protein P022 is referred to as an example of a (native) DsA1 polypeptide, P027 is referred to as an example of a (native) DsA2 polypeptide and P028 is referred to as an example of a (native) putative iron-transport protein (PITP) polypeptide of P. acnes.

The Rationale for the Selection of DsA1 and/or DsA2 as Antigens:

The current challenge in the field is to select the antigens most suitable as vaccine material among many different candidates that were studied in the prior art and suggested as potentially relevant either as a virulence factor or as a potential material in developing a therapeutic or prophylactic treatment. The lack of systematically conducted, comparative studies in which their actual performance as vaccine candidates could be analysed and compared to each other for their ability to induce protective immune responses against the bacterium, makes it difficult to make a final selection.

In the course of the present invention a large number of the proteins suggested to be involved in P. acnes virulence and/or considered potential candidates for an immunotherapeutic product were tested. Although all prior-art antigens were capable of inducing immune response in the murine immunization studies according to the present invention, as evidenced by their ability to induce antigen-specific antibodies detectable in ELISA (e.g. FIG. 2), this however did not translate into comparable level of surface binding and opsonophagocytic activity.

To address one or more of the objects of the present invention, a method for producing a vaccine for use in the treatment or prevention of P. acnes-associated infestions is provided, comprising
selecting at least one antigen comprising at least one P. acnes epitope, wherein the P. acnes epitope is an epitope which is directly accessible to human serum antibodies when presented by the live P. acnes bacterium and the specific binding of the epitope by the human immune system leads to reduction of bacterial numbers, fitness, growth, virulence or a combination thereof,
   wherein binding of the epitope by the immune system, especially by the human immune system, is evidenced by using a flow cytometry assay, preferably by fluorescence-activated cell sorting (FACS) assay;
   and
   wherein reduction of bacterial numbers, fitness, growth, virulence or a combination thereof, especially the reduction of bacterial numbers (due to the direct effect of antibodies), is evidenced by an opsonophagocytic killing assay (OPK) in the presence of antibodies specific for at least one antigen comprising at least one P. acnes epitope.

The method for producing a vaccine for use in the treatment or prevention of P. acnes-associated infections according to the present invention, comprises
selecting at least one antigen comprising at least one P. acnes epitope, wherein the P. acnes epitope is an epitope which is directly accessible to human serum antibodies when presented by the live P. acnes bacterium and the specific binding of the epitope by the human immune system leads to reduction of bacterial numbers, fitness, growth, virulence or a combination thereof,
   wherein binding of the epitope by the immune system, especially by the human immune system, is evidenced by abtibody binding to P. acnes bacteria using a flow cytometry assay, preferably by fluorescence-activated cell sorting (FACS) assay, and by binding of antigen-specific antibodies to live P. acnes bacteria in the flow cytometry assay;
   and
wherein reduction of bacterial numbers, fitness, growth, virulence or a combination thereof is evidenced by an opsonophagocytic killing assay (OPK), wherein said OPK assay shows at least 50% more reduction compared to negative control in an at least two-fold higher dilution as used in the opsonophagocytic killing assay for Kso titer determination, of bacterial cell counts in a reaction sample, after an incubation of at least 24 h or longer at 37° C., in the presence of 5% CO₂, using at least two serial two-fold dilutions of a polyclonal human antibody specific for the at least one antigen comprising at least one P. acnes epitope, or human serum containing such antibodies, or an antigen induced polyclonal antibody serum; starting at complement-inactivated serum dilution of at least 1/200.

With the present method, for the first time “real world” P. acnes antigens are identified and provided resulting in a completely new understanding of the nature of the human P. acnes microbiome interactions with the host and—based thereon—providing new and efficient “real-world” strategies for combatting and preventing disorders caused by pathological consequences of the colonialization of humans with P. acnes bacteria. In contrast to other strategies proposed in the prior art, the identification of antigens according to the present invention provides information to the relevance of the antigens and the specific epitopes tested by antigen induced polyclonal antibodies.

With the present invention, a real world relevance of vaccine candidates was safeguarded by actually testing if the antigens are really able to induce the antibodies that can bind the surface of many different strains (as opposed to guessing based on indirect evidence/bioinformatics or methods which only look at the expression of the proteins in the cell lysates, e.g. by Western Blot or MasSpec; or relying on the irrelevant models that do not accurately represent human disease (e.g. CAMP and sialidase studies in rabbit ear inflammation), especially wherein no flow cytometric studies have been done on antigen expression evaluation in P. acnes (as the only quantitative way to evaluate differences in the surface accessibility of antigens and their epitopes to the antibodies raised by immunization)). In the course of the present invention, a systematic study of a large number of different strains and phylotypes (more than 100, not only 1-6 strains as done by others) was performed. Moreover, clinically relevant material was isolated from the pustules (inflamed lesions) of acne patients, after sterilizing skin surface and analyzing them separately as opposed to prior art where the samples are taken from the areas not typically affected by acne, from the skin surface and during the analysis they analyzed the data of all individuals as a group, e.g. acne vs. healthy (but each individual is unique in terms of its microbiome, and putting all data “into the same bag” only skews results and leads to wrong conclusions)). In addition, a real-world relevance was also shown in terms of the significance of human immune responses (that increased binding to P. acnes surface leads to increased bactericidal efficacy of phagocytes; and that for this, the antibody binding has to be directed to the correct epitopes). This approach showed how it also works in humans (that surface binding and OPK data also correlated when human sera were tested). Finally, the uniqueness i.a. of the OPK method applied in the course of the present invention has proven also its significance for the adaptive immunity, whereas the prior art has only looked into the innate immunity based on interactions of P. acnes and human cells in the absence of antibodies.

In fact, the present invention has overcome the limitations in the field related to the methods used to identify and select the best antigens and epitopes for inclusion in a vaccine.

This invention provides the “real world” evidence for the surface expression and differences in the accessibility of different antigens and antigenic epitopes across a large number of P. acnes strains (Table 2); and the ‘real world’ evidence of the functional consequence of the binding for inducing effective antigen-specific adaptive immune response (which is the key criterion for vaccine antigen selection) based on the quantitative evaluation of the binding and its functional outcome by using the methods superior to others in the prior art:
Flow cytometry
Opsonophagocytosis
SPR/Biacore Flow cytometry: Systemic evaluation of a large number of different P. acnes strains and antigens with the objective to quantitatively evaluate and confirm the actual expression of different antigens on the bacterial cell surface and the accessibility of antigenic epitopes to binding of antigen-specific antibodies, has not been previously done in the P. acnes prior art. Instead, antigen expression was evaluated indirectly using sub-optimal methods and only small number of P. acnes strains (e.g. Western Blot in Lodes et al., 2006). None of these methods had quantitatively evaluated the levels of expression of the proteins on the cell surface (the detection of the protein after the separation by SDS-PAGE and Western Blot, does not imply that the protein is actually expressed on the cell surface and sufficiently accessible to the antibody binding on the surface of P. acnes—these methods do not allow the comparison of the levels of expression across different strains and different antigens to each other). The antigenic epitopes need to be not only accessible on the surface, but also sufficiently conserved among a large % of strains, in order to be suitable as vaccine candidates (a vaccine has to be able to provide protection to as many patients as possible, therefore, should induce antibodies capable of recognizing all strains that may become virulent and that may colonize different hosts).

Other researchers have studied antigen expression in cell lysates, cell secreted fractions and/or membrane fractions analyzed by Western Blot or Mass Spectrometry (Holland et al., 2010; Yu et al., 2016; Bek-Thomsen et al., 2014; Achermann, 2015; Lodes, 2006), or evaluated the immunogenicity of different antigens expressed by only one P. acnes strain during the infection of rabbit bone tissue (their ability to induce antibodies against the recombinant proteins) (Achermann et al, 2015). All these methods had similar limitations, that a minor number of strains were tested, the proteins that were analyzed were denatured and separated by the SDS-PAGE; therefore, the proteins are taken out of the context of the live cell, its native epitope structure has been changed and so for example. Achermann et al, 2015 did not identify any of the antigens described in this invention.

Without the results presented in this invention, a skilled person in the art could not conclude that the most effective vaccine against P. acnes would need to be highly cross-reactive and that combining DsA1 and DsA2 as single antigens or in a hybrid in combination with P028 (PITP) provides a much wider cross-reactivity than a product that would incorporate P022 and P027 each as a single antigen vaccine or in a combination without P028. This is especially relevant considering individual-specific and unique profile of strains colonizing each individual, as well as expected variations of in vivo expression of single antigens in the context of different pathologic processes and virulence mechanisms which this bacterium employs.

Opsonophagocytic killing method: OPK method allows evaluation of functional significance of antigen-induced antibody responses in mobilizing adaptive immune defenses against the bacterium, that specifically lead to reduction in the bacterial cell numbers and proliferation at the sites of the pathogenesis.

Since P. acnes does not colonize animals or is not pathogenic in animals, the pharmacodynamic studies typically performed for vaccines are not relevant in animal models, e.g. such as protection against challenge. The published animal models in which P. acnes was used to induce inflammation in the rabbit ear (e.g. Liu et al., 2011) are not accepted by the scientific community neither as relevant for acne vulgaris, nor as relevant for the identification of vaccine antigen candidates: only one single antigen (Camp2) was studied since the model did not allow objective comparison of its performance to other P. acnes antigens (O'Neil and Gallo, 2018; McLaughlin, 2019). Moreover, Liu et al., 2011 did not even consider bactericidal activity as a desired vaccine mode of action, instead they state that: “neutralization of bacteria-induced virulence and inflammation without directly killing bacteria would be an excellent immunotherapeutic for the treatment of acne vulgaris.” (Liu et al., 2011; page 3, first paragraph).

The ability of a serum sample to opsonize bacteria can be measured by various in vitro opsonophagocytic killing (OPK) assays which have been shown to be superior to simple opsonophagocytic uptake (without killing) OPA assays and the best functional correlate of protection.

Although granulocytes (neutrophils, basophils, eosinophils) from peripheral blood can be used as a source of phagocytes for opsonization assays, using cell lines as phagocytes is more convenient and reproducible. Promyelocytic leukemia cell lines, such as HL60, can be induced to differentiate into granulocyte-like cells and the differentiation can be monitored by the expression of surface antigens.

These assays are not trivial and require development of the assay to account for unique properties of the specific bacterial species and the nature of its interactions with human immune system. Despite the success of OPK and OPA assays use in evaluation of vaccine antigen-induced responses against S. pneumoniae, and recently a similar assay has been developed against S. pyogenes, no such assay has been developed against P. acnes. The difficulty in establishing such an assay for P. acnes was especially due to the uniqueness of its cell biology, cell wall properties and its unique interactions with human immune system as a commensal bacterium that is an opportunistic pathogen.

No further studies have attempted to evaluate the role of antibodies as important adaptive immune defense against P. acnes-associated infections that could lead to bactericidal effects. Instead, over the last two decades, the scientific community had studied a general phenomenon of P. acnes interactions with different cell types, independent of antibodies and attempted to understand P. acnes fate upon internalization or invasion of these cells. These studies only provided the insights into innate immune defenses against P. acnes, but did not solve the question of how infection and/or pathogenic insult by this bacterium can be prevented or resolved by the adaptive immune defenses, when the innate immune response is not able to stop it and which antigens are able to induce the antibodies with bactericidal activity against P. acnes.

The main differences in the OPK assays developed for P. acnes in the course of this invention are e.g.:

(1) Preferred use of most relevant cells for the assay: Granulocytes and especially neutrophils (HL60 cells differentiated into granulocytes). Granulocytes and especially neutrophils, play the important role in early host defense against invading bacteria. In response to infection, neutrophils are recruited from the bloodstream and migrate toward the local sites of inflammation where they aid local innate immune defenses in order to stop or prevent the infection spreading.

The specifically preferred cell type used, HL60 cells, are non-adherent cells easily mixed in solutions and this allows performing the assay using a constant cell number in all wells, which significantly reduces variations in the assay that make it difficult to compare in parallel the performance of a large number of test samples.

(2) Preferred use of antibodies raised specifically against different antigens in order to investigate their role in inducing phagocytic killing by the cells used in the assay (e.g. preferably HL60 cells differentiated to granulocytes).

(3) The phagocytic killing is preferably evaluated under the conditions which do not include antigen non-specific killing, which may occur in the absence of antibodies in the studies where antibiotics or complement were used. This ensures unbiased evaluation of the potential of different antigens and antigenic epitopes to induce antibodies with bactericidal activity.

(4) The phagocytic method is preferably optimized so to ensure optimal P. acnes growth and survival of both extracellular P. acnes as well as HL60 phagocytes over the extended incubation period of more than 24 hours: in other methods the time during the killing phase is 24 h or lower, and if longer time is allowed, antibiotics are used to prevent outgrowth of extracellular bacteria or the assay variability increases making it difficult for systematic evaluation of a large number of samples in the same assay.

SPR/Biacore: is preferably used according to the present invention as an additional quantitative method for antigen selection included evaluation of the antibody binding levels and affinity (stability of interaction) with different antigens selected based on immunorelevance (in surface binding and opsonophagocytic assays).

The skin is a complex and dynamic ecosystem that is inhabited by the skin microbiota; bacteria, archaea, fungi and viruses. Skin-resident bacteria are not just passive residents; they actively engage host immunity through an intact skin barrier and activate specific immune cell populations in a species- and strain-dependent manner.

Over the last decade it became evident that skin has its own skin immune system (SIS) and that although most antibodies act systematically by blood distribution throughout the body and diffusion into tissues, tissue titers can be enhanced by localized antibody production by the B cells that reside in the skin. In mammalian skin, B cells localize to the dermis. In human skin.

IgA is secreted in eccrine sweat glands and appears in sweat and sebum, and antibodies of IgG and IgM isotype reach the skin surface by undefined mechanisms.

Despite these findings, no other studies in the prior art have attempted to address the need to increase the local concentration of antibodies against P. acnes by stimulating their generation and increasing bactericidal activity. Instead, the field has moved in the direction that increasing the antibody levels against the bacterium may be detrimental, rather that beneficial, and if such strategy is considered, the caution is raised about the unwanted effects and the secreted, non-surface proteins are considered as more optimal targets.

According to the present invention the amount and quality of P. acnes-specific antibodies which are locally secreted and which diffuse from the blood capillaries into the hair follicles is increased. These antibodies strengthen local skin immune defenses and increase their efficiency, so that the inflammatory process is prevented or significantly reduced (FIG. 24A, 24B).

The pathogenesis of acne is triggered by the interplay of several factors, including genetics, hormonal activity, skin environment conductive to P. acnes virulence and the resulting immune response. This is relevant especially in view of the commensal vs. pathogenic role of skin-colonizing bacteria. The complexity of the interactions among different environmental, bacterial and host factors result in different individual clinical manifestations of the disease and responsiveness to the currently available treatments. Other P. acnes-associated pathological conditions and diseases involve similarly complex interplay of the interactions, because the mere colonization by a specific strain is not enough to induce the infection, inflammation and disease.

For this reason, over the last several decades, the scientific community has been mostly focusing on understanding the pathogenic process that leads to different clinical manifestations, in isolating P. acnes from infected tissues and lesions, deriving conclusions about their pathogenic significance and then comparing the expression of different virulence factors expressed by the ‘virulent’ strains.

In fact, prior art studies have presented only the evidence of the expression of antigens by specific phylotypes, compared the expression levels of different proteins inside the skin pores of acne vs. healthy or examined their ability to interact with human proteins and different components of the human immune cells and tissues; however they left many questions opened in terms of the vaccine optimal antigen selection and design, so to ensure benefit for the patients without causing unwanted effects by harming the more “health-associated” P. acnes strains.

In the scientific research in the present field, the essential questions of how to design a suitable vaccine for P. acnes-associated pathological conditions has still not been answered. In fact, the scientific community has even moved away from considering a vaccine for treating P. acnes-associated diseases a feasible approach. The biological function and expression of DsA1 and DsA2 in the context of human disease has been studied by many groups, however there is still no proposal for a vaccine, let alone a vaccine that is based on these two proteins and designed so to induce antibody responses to target specific epitopes of these proteins. Moreover, lots of caution is being raised about the vaccine approach that would target P. acnes surface-associated proteins. In summary, the present invention provides “immunorelevant” vaccines which differ from identifying “virulence factors” in the prior art (which indicates only that these proteins are involved in the interactions with the host immune system leading to inflammation). In contrast to such “virulence factors”, the “immunorelevant” vaccines according to the present invention refer to their relevance as a vaccine candidate which upon immunization induce antibodies which significantly increase the ability of the phagocytic cells against the bacterium, to prevent their proliferation and virulent behaviour. Accordingly, “immunorelevant” antigens in this invention are not inducing but rather reducing and acting against the inflammation; therefore, having a protective effect as a vaccine in the host.

Also in contrast to OPK assays in the prior art, the OPK assay according to the present invention is preferably performed
without the presence and/or addition of antibiotic compounds to the assay and/or
without the presence and/or addition to complement and/or complement factors;
   To allow unbiased analysis of the quality of the antigens as such for the efficient killing of the P. acnes bacteria without systematic influence of antibiotics or the complement (the innate immune system) on the bacterial killing in the OPK assay.

Although previous studies referred to screens for “surface-based antigens, the present invention uses, as a first step, flow cytometry to select the most appropriate and best candidate which are then further subjected to an OPK assay. Although Nakatsuji et al, 2008 used a flow cytometry method to study the binding of recombinantly expressed sialidase to sebocytes, however, it was not even demonstrated that the sialidase is expressed at all on the surface of P. acnes and accessible to antibodies induced by immunization. In fact, in the prior art only bioinformatics prediction was used to select antigens which may be associated with the cell wall. It is worth mentioning that even the group behing Nakatsuji et al, 2008 later changed their strategy and concluded that targeting surface-proteins is not the best approach for P. acnes, because they did not want to target the entire bacterium which could have negative consequences for health (since it is a comensal) and continued to pursue CAMP2 (secreted protein) as their preferred vaccine antigen (Liu et al., Vaccine 29 (2011), 3230-3238): The authors concluded with respect to their previous proposal to use CAMP as a vaccine target that although “vaccines targeting a surface sialidase or bacterial particles exhibit a preventive effect against P. acnes, the lack of therapeutic activities and incapability of neutralizing secretory virulence factors motivate us to generate novel immunotherapeutics”. Consequently, in the Liu et al., 2011, article, the scientists skipped CAMP2 as an active vaccine target and developed an immunotherapeutic antibody to secretory CAMP factor of P. acnes.

The OPK assay according to the present invention can be performed with any cell types capable of uptake and killing of P. acnes bacteria and are preferably performed with granulocytes (especially neutrophils and basophils), although polymorphonuclear cells purified from the blood of healthy volunteers and certain types of macrophages, monocytes, and other phagocytic cell lines may be used. Preferably, the OPK assay is performed by mixtures of phagocytic cells (granulocytes), especially in the presence of serum or other tissue liquids containing (polyclonal) P. acnes-specific antibodies. Preferred cell lines used in the OPK assays according to the present invention are HL-60 cells, human myeloblast and promyelocytic leukemia cells, which are differentiated into granulocytes (including, neutrophils which are the main cell types responding to infections and migrating from blood to the sites of local inflammation, the process dependent on the cytokine immune signals that are released by the local resident cells and macrophages in response to tissue injury and when the mechanisms of innate immunity cannot do the job of clearing and preventing the spread of the infection). Neutrophils and/or granulocytes are the most preferred cell type to be used in the present OPK assay.

Antibodies induced by DsA1 and DsA2 consistently bound P. acnes cell surface, this is surprising and in contrast to other prior art proteins, which were also suggested to be important virulence factors, immunogenic and exposed on the cellular surface and/or secreted (P002, P005, P035, P042, P046, P068, P069, P070, P071). Indeed, none of these prior art antigens were detected by a flow cytometry-based surface binding assay of the present invention on representative strains of all 6 MLST phylotypes or the surface binding of antibodies induced by these antigens was extremely low (e.g. FIG. 1A).

Despite the high potential for secretion, variability in the expression and differences in the antigenic sequence as suggested in the prior art (Lodes et al. 2006: Holland et al. 2010; Yu et al. 2016; McLaughlin et al. 2019), in the surface binding experiments of the present invention, it could be shown that DsA1 and DsA2 are not only expressed within the cell wall fraction or on the cell surface of a limited number of strains or phylotypes, but that they are highly accessible to the antibodies raised by immunization when used as a vaccine candidate and that this binding is far superior in the intensity and cross-reactivity/cross-type-reactivity compared to other proteins described in the prior art (O'Neill and Gallo 2018; McLaughlin et al. 2019) (e.g. FIG. 1A, FIG. 4). The expression and accessibility of these antigens on the cell surface of live P. acnes is not only significant on the cell surface of the phylotype IA1 (McLaughlin et al. 2019), but also the level of their expression on the bacterial cell surface of the phylotypes IA2, II and IC is by far superior compared to other antigens described in the prior art (Holland et al. 2010; Brzuszkiewicz et al. 2011; Bek-Thomsen et al. 2014; Achermann et al. 2015; Yu et al. 2016; O'Neill and Gallo 2018; McLaughlin et al. 2019).

Moreover, the significance of the surface-binding of antibodies could be confirmed in the present invention in an opsonophagocytic killing assay using human phagocytic cells which demonstrated that only the antibodies that act against the antigens DsA1 and DsA2 were able to induce significant opsonophagocytic killing of P. acnes, whereas the antibodies induced by immunization using other prior art antigens were clearly inferior to the antigens DsA1 and DsA2 when examined against a panel of genetically different strains, representing most common MSLT groups (e.g. FIG. 1B), namely IA1, IA2, IB, IC, II and III, thereby evidencing cross-reactivity and cross-type reactivity.

Therefore, although similar to other suggested virulence factors that were suggested to be located on the cell surface of specific phylotypes or capable of secretion and transient expression within the bacterial cell wall, only DsA1 and DsA2 had displayed the key property of a protective immunogen and vaccine candidate: they were able to induce antibodies capable of binding to the surface of live P. acnes bacterium in sufficient quantity and the quality necessary for the induction of significant opsonophagocytic killing capacity.

Both proteins represent virulence factors in P. acnes, required for the infectious P. acnes cycle. They have been suggested to bind specific host factors, such as dermatan sulfate (Lodes et al. 2006) and fibrinogen (Grange et al. 2017), however a person skilled in the an as of current state in the field, could still not conclude how to use either of these proteins to design and develop a protective therapeutic product. Instead, caution is being especially raised related to the antigens that are associated with cell surface of a large percentage of P. acnes strains, because such a vaccine is thought to be dangerous because it would induce unwanted effects against the ‘commensal’ strains, so the teaching in the prior art instructs to either select primarily among the antigens expressed by MLST group IA1 (McLaughlin et al. 2019, page 23); or to select the antigens that are secreted and not associated with the bacterial cell surface, (Contassot 2018; Keshari et al. 2019), and these authors did not even mention DsA1 and DsA2 among different antigen candidates that could be evaluated in future studies to determine their potential as possible vaccine antigen candidates.

DsA1 and DsA2 are homologous to each other, and they are differentially expressed on the strains within the phylotypes IA1, IC and II, so that some express more of the one compared to the other homologue (e.g. FIG. 16A-16D). It is also possible that one can take over the function of the other one in case they are targeted separately. Therefore, providing at least one of the two or even better, both of them in a vaccine can decrease the chance of immune defense evasion and increase the vaccine potential against the strains expressing lower levels of one of the two.

Various virulence functions have been ascribed also to other proteins suggested as important by different authors (Brzuszkiewicz et al. 2011; Achermann et al. 2015; O'Neill and Gallo 2018; McLaughlin et al. 2019); as a multitude of the factors expressed by the bacteria add up to their pathogenic potential. Therefore, what makes this invention unique is not the identification of the proteins as one of many different virulent factors contributing to invasive potential of specific P. acnes genetic types and interacting with the host, but the evidence that the combined action of the DsA1—and DsA2-specific antibodies induced by immunization using them as a vaccine material is able to mobilize the host defenses (phagocytes) and specifically induce and increase their killing capacity towards a very large number of bacterial strains and phylotypes, to prevent or heal the injury of the skin hair follicles, or to prevent their further spread within the organism when they manage to breach the skin barrier. The induction of such potent opsonophagocytic killing effect obtained in the course of the present invention makes these two proteins the prime candidates for a vaccine product, unlike many other virulence factors that have been suggested as potential candidates in the prior art.

### The Rationale for the Selection of PITP as Vaccine Antigen

Antigen P028 or PITP (PA-21693) had been described by Lodes et al. (2006) as one of the two putative P. acnes proteins (PA-21693 and PA-4687) that were similar to the product of the Corynebacterium diphtheriae htaA and involved in the iron uptake mechanism by P. acnes.

The present data demonstrated that under iron limiting conditions, the expression of PITP was significantly increased, whereas the expression of other surface proteins remained unchanged under the same conditions; and the expression of the second htaA-like iron-binding protein which has been also suggested by Lodes et al. (2006) as one of the three variably expressed and immunogenic P. acnes proteins. P071 (PA-4687), only slightly increased under iron limiting conditions on some strains (e.g. FIG. 3). Iron is essential for the replication of bacteria and especially under inflammatory conditions, because iron uptake plays a role in bacterial survival in the host tissue.

Under the iron-limiting conditions PITP was detected in variable amounts on the surface of six MLST phylotypes (Type IA1, IA2, IB, IC, II and III) (e.g. FIG. 1A). PITP is highly immunogenic, and the induced immune response recognizes and specifically binds the strains that belong to all 6 MLST types. The induced immune response was also able to induce significant bacterial killing by the phagocytes in the OPK assay against at least two or more different MLST phylotypes (e.g. Type IA1 and IA2, or IA1 and IB, or IA1, IA2 and IB, or IA1, IA2, IB, IC and/or II and/or III) (e.g. FIG. 1B). Therefore. PA-21693 (Lodes, et al. 2006) (PITP) as a vaccine antigen had demonstrated within the course and assessments of the present invention clear superiority in the ability to induce cross-reactive and cross-type-reactive P. acnes antibodies that bound the cell surface of a wide range of MLST phylotypes (e.g. FIG. 1A) and in the ability to induce phagocytic killing (e.g. FIG. 1B), contrary to other similar iron-binding proteins, such as P071, an Hta-like protein PA-4687 (Lodes et al. 2006), that was also suggested to be highly immunoreactive P. acnes surface protein (Lodes et al. 2006), and which was considered among important virulent factors and potential immunotherapeutic candidates against Type IA1 strains (McLaughlin et al. 2019).

Due to the fact that IB strains are isolated from the skin of both acne patients and healthy individuals, in the prior art their pathological significance has not been recognized (O'Neill and Gallo 2018; McLauglin et al. 2019) and IB strains in acne vulgaris are considered among ‘commensal’ or ‘neutral’ strains, since they are equally frequent in both healthy and acne skin. However, sporadic clinical evidence suggests that this may not be always the case. For example, the strain found to be responsible for the acne therapy failure due to antibiotic resistance was of IB phylotype, whereas the strains considered ‘virulent’ (IA1) were sensitive to antibiotic used in the treatment: acne symptoms were reduced only when the antibiotic was exchanged for the one that was effective against IB strains (Sadhasivam et al. 2016). Therefore, the clinical significance of IB phylotype in acne has not been resolved in the field, although type IB strains are frequently isolated from other types of P. acnes infections. However, the present invention points that IB strains are also isolated from the inflamed acne lesions (e.g. FIGS. 14A, 14B) and that humans develop significant level of antibodies against IB strains, which may reach the opsonophagocytic serum titer (K50) of more than 1 million (e.g. FIG. 13B).

Type III strains, although not frequently isolated in association with acne (McLaughlin et al. 2019), also have pathogenic potential as evidenced by their isolation from implant-associated infections and other types of P. acnes infections, and they have been also suggested to be specifically associated with progressive macular hypomelanosis (Barnard et al. 2016; Dagnelie et al. 2018). The reason for their less common isolation from the skin of acne patients is that they tend to be attracted to different environment and colonize more limited areas of the face and body: in published studies they were identified only on forehead and forearm of some individuals (Dekio et al. 2012) and on the oral mucosa (Scholz et al. 2014). The data generated in the course of the present invention also show that Type III strains may prefer to colonize more limited bodily areas since Type III strains were not isolated from the facial areas sampled in the study according to the present invention from either healthy or acne patients. In addition, it was noticed that Type III strains also grow more slowly than other phylotypes, so depending on the type of sampling method and the area being sampled, when the isolated clinical material is propagated on the plates prior to sequencing, they can be easily missed.

PITP therefore is a suitable surface antigen for targeting additional P. acnes types with virulent potential (e.g. IB and/or III) as well as adding to the effects of DsA1 and DsA2 on the additional phylotypes where the amount of their expression is lower or may be downregulated (e.g. FIG. 4).

The interaction of an induced humoral immune response with PITP, besides the attraction of immune cells, could also lead to the inhibition or reduction of iron-uptake by the bacterium during its fight with the host immune cells or interfere with biofilm dispersion and spread of infection.

### Rationale for a Combination of DsA1+DsA2+PITP in a Single Vaccine Product

Antigen PITP by its surface accessibility and OPK activity profile, best complements the immune response induced by either of the antigens DsA1 or DsA2: it is accessible on the cell surface and able to induce antibodies capable of phagocytic killing of P. acnes types IB and to a lower degree Type III which do not express either of the other two antigens, and on the strains where PITP surface expression may be lower compared to DsA1 and DsA2 (P. acnes types IA2 and II), antibodies induced by the other two antigens can compensate for that (e.g. FIG. 1A, 1B; FIG. 4, FIG. 5).

The functional significance of opsonophagocytic killing (OPK) is the key inventive step of the present invention due to the fact, that many other proteins have the ability to act as virulence factors but could not induce antibodies with the same effects against the bacterium when tested in comparison to each other in the same immunological assays. For example, CAMP2 and its homologues, have a hemolytic activity which can lyse human cells: CAMP2 has been recently reported to be able to induce killing of phagocytic cells in an in vitro assay of co-cultivation with P. acnes (Wang et al. 2018). The expression and secretion of CAMP proteins by different P. acnes strains has been suggested one of the factors contributing to P. acnes resistance to opsonophagocytic killing. However, as evidenced in the OPK assay performed according to the present invention, killing of P. acnes was consistently detectable in the presence of opsonizing antibodies against DsA1, DsA2 and PITP antigens.

The studies performed for the present invention in a flow cytometry based surface binding assay showed that the best effects can be obtained by combining antigens DsA1 and/or DsA2 with the antigen PITP (e.g. FIG. 5). This could be confirmed by the opsonophagocytic killing that could be induced with the antibodies against PITP also against the strains from the phylogroups against which the antibodies induced by DsA1 and DsA2 did not induce opsonophagocytic killing, e.g. MLST phylotypes IB and III (e.g. FIG. 1B, 1D). Therefore, using a combination of DsA1 and/or DsA2 with PITP provides a wider cross-reactivity/cross-type-reactivity than a product that would incorporate only DsA1 and DsA2 used as single antigens or in a combination. This is especially relevant considering individual-specific and unique profile of strains colonizing different individuals and different pathogenic potential of the strains in context of different pathologies in which this bacterium is or may be involved: different types of acne (acne vulgaris, acne conglobata, cystic acne), implant infections, sarcoidosis, progressive macular hypomelanosis and other types of conditions. As evidenced by the examples of the present application, the vaccine products according to the present invention are inducing antibodies with a broader cross-reactivity and cross-type-reactivity profile directed against the virulence factors that are expressed on a wider number of phylotypes, has a much higher potential to be protective in a high percentage of the individuals at risk and in different pathological conditions.

This specific combination of PITP with either DsA1 or DsA2 makes it possible to target not only the strains that are classified under ribotypes suggested by others to be pathogenic in acne vulgaris (RT4, RT5 and RT8) (Fitz-Gibbon et al. 2013: O'Neill and Gallo 2018), but also those strains that have the same capacity to express these virulence factors within other ribotypes (e.g. FIG. 4: RT1 and RT3), but which pathogenic significance has not been so far recognized due to the fact that they are also frequently found on healthy skin (e.g. RT1 and RT3) or because they have not been isolated to a significant extent in the context of infection (e.g. FIG. 4: RT16 and RT532)

Although ribotyping and other similar genetic typing schemes may associate in some cases with the immunological data, this association does not imply the causation. The data obtained with the present invention demonstrate that the surface accessibility of the selected vaccine candidates is significantly lower on the ribotypes which are not commonly isolated from acne-prone skin (RT2 and RT6), and tends to be higher on those strains that have been implicated in acne pathogenesis (RT4, RT5, RT8): however exceptions were detected in the course of the present invention in which a completely different surface expression pattern is detected on the strains that are classified under the same ribotype (e.g. FIG. 4, strains 79 and 87 within RT1, or strain 80 within RT5).

A vaccine targeting 3 different antigens is also a strategy to reduce occurrence of potential escape and resistance mechanisms, which bacteria may employ by downregulating one or two of the proteins during its infectious cycle.

Rationale for Making a Fragment or a Hybrid Molecule of DsA1 and/or DsA2

The aim of a preferred strategy according to the present invention was to find a hybrid protein able to induce antibodies with the surface binding and functionality in the opsonophagocytic killing assay as comparable and as broad as possible to antibodies induced by the combination of both full-length proteins DsA1 and DsA2 for the purpose of simplicity and lower cost of production.

Several fragments of DsA1 have been produced and used for immunization studies. Surprisingly, fragments of DsA1 which represent primarily the C-terminus of the protein (e.g. a fragment termed “F4”) induced antibodies which poorly bound P. acnes cell surface (e.g. FIG. 6, FIG. 18A) and were not able to induce bacterial killing in the opsonophagocytic assay (FIG. 18B, 18C), which is in a stark contrast to the authors that emphasized high immunogenicity and immunological importance of the C-terminus containing the PT repeat domains of DsA1 (Lodes et al. 2006; Grange er al. 2017).

Bioinformatics analysis, after determining the biological/clinical and immunorelevant significance of the targets in the course of the present invention, revealed a 3-domain structure of the protein (referred to herein as “CSD1”, “CSD2”, and “CSD3”) which was used as a tool to optimize the design of fragments and hybrids to be tested in immunological studies based on the sequence areas that provided some flexibility in terms of start and end of a specific fragment (e.g. FIG. 7A,/C). The used ‘swap sites’, i.e. the positions where DsA1 and DsA2 sequence regions were switched (as in a protein-cross-over) were chosen within swap regions, areas of least likelihood to interfere with structural elements, specifically alpha helices (e.g. FIG. 7B).

Based on the insights from the immunological studies (e.g. FIG. 6) and bioinformatics analysis, additional fragments and hybrids of DsA1 and DsA2 have been designed to further investigate the most important epitopes of these proteins which are used as immunogens to induce antibodies capable of equally good specific binding of both DsA1 and DsA2. Serum antibodies induced e.g. by the hybrid H4 molecule that includes the functionally more relevant regions confined mostly to the N-terminal and central part of the protein molecule of DsA1 (amino acids 29-145; 278-333) and only the central part of the DsA2 protein sequence (amino acids 190-321) (FIG. 12A), had the ability to specifically bind to both full length proteins in an ELISA assay (e.g. FIG. 8). Likewise, another example for a hybrid molecule, the hybrid H2 molecule that included the two central and homologous regions of DsA1 (amino acids 146-277) and DsA2 (190-321) had also induced the antibodies with a comparable ability to recognize both DsA1 and DsA2 full length proteins (e.g. FIG. 8). Immunization with hybrid H4 induced antibodies which showed the most balanced specific binding against both protein DsA1 and protein DsA2, in contrast to antibodies induced by immunization with full length proteins and other fragments and hybrid versions, which specifically bound most highly against themselves but to a much lower extent to the second antigen (homologue). Similar conclusions were obtained based on the analysis of the intensity and stability of interactions by the surface plasmon resonance technology (SPR) performed on Biacore (FIG. 19A, 19B).

In surface binding assays on a collection of strains from different phylotypes, antibodies induced by immunization with hybrid H4 had the broadest and most balanced cross-reactivity and cross-type-reactivity among the hybrid examples tested according to the present invention (e.g. FIG. 9).

Antibodies induced by immunization with other versions of hybrid constructs (e.g. H3 and H5), as well as with newly designed and additionally optimized C-terminal fragments (F12, F13) and the N-terminal fragments (F10) were significantly less cross-reactive compared to antibodies induced by immunization with hybrid H4 when tested as single vaccines or within the design of other hybrid molecules, e.g. hybrid H3 (e.g. FIG. 9). This is also in contrast to the knowledge in the prior art: the studies of similar proteins found in other bacteria, such as protein M (Fischetti 1989), demonstrated the crucial importance of the C-terminus for the induction of immune response with a high level of cross-reactivity among many different serotypes and strains, whereas the data obtained in the course of the present invention have shown that despite the similarity to streptococcal proteins, the C-terminus of DsA1 and DsA2 is not immunologically relevant, because the fragments which contained primarily C-terminal part of the protein sequence, performed inferior compared to fragments and hybrids containing the more central parts of the molecule.

Multiple sequence alignment of DsA1 (native) polypeptides from different P. acnes sources has revealed that DsA1, while generally thought of as hypervariable protein, is in fact highly conserved and essentially invariable. Among existing variabilities only PT-variability may be understood as true protein-variability, specifically because the transient nature of observed genomic frame-shifts has not been determined so far and the effect of N-terminal frame-shifts is essential non-expression. The other exception may be the rare C-terminal loss of the anchorage motif, but that according to current data is either very rare or limited to genes not expressed as proteins. Importantly, this invariability does not suggest or prioritize any part of the protein over another. Specifically, when carefully considering the assembly errors in public repositories even typical PT-length variability may be exaggerated. Moreover, if anything, the length variability of the PT region could be understood as an effect of immunological pressure and has been interpreted as such in the literature. In the course of the present invention, it was shown that this is incorrect, in fact the experimental results disclosed herein demonstrate the opposite: PT is immunologically largely irrelevant. It may be understood as a flexible, possibly glycosylated linker tethering the N-terminal domain(s) to the cell-surface. This further solidifies that sequence variability does not obviously suggest any specific part of the DsA1 sequence as vaccine candidate and such interpretation (in the case of PT) is in fact incorrect.

Therefore, contrary to the prior art, structural epitopes and epitopes within a more central region of DsA1 (amino acids 146-277) and DsA2 (amino acids 190-321) sequence were found in the course of the present invention to be much more immunologically relevant as vaccine antigens or epitopes, since the fragments containing these regions when used as vaccine antigens induced much more cross-reactive/cross-type-reactive antibodies that bound the surface of P. acnes much better (e.g. FIG. 9: H4, F11, H2, F9, P027-F1), compared to the fragments and hybrids which lacked this part of the sequence or contained only a part of it (e.g. FIG. 6: F3, F4; and FIG. 9: F10, F12, F13). When different hybrids and constructs were compared to each other in the opsonophagocytic assay against Type IA1 strain, the fragments and hybrids containing more central regions of DsA1 and DsA2 induced very high levels of opsonophagocytic killing at almost all serial dilutions, hybrid H3 that contained N-terminal sequence regions of DsA1 (amino acids 29-145) and DsA2 (amino acids 72-189) induced significant opsonophagocytic killing up to 1/40,000 serum dilution, whereas the C-terminal fragment, F4 was negative at all tested dilutions (FIG. 18 B, 18C). Moreover, the hybrid H4 induced ≥95% of the opsonophagocytic killing at all dilutions, even at 1/1,280 000, therefore performing even better that the serum antibodies raised against full-length DsA1 and DsA2 proteins (e.g. FIG. 18B, 18C).

Despite the significantly higher level of conservation than suggested in the prior art, DsA1 and DsA2 do show variable amounts of expression on some strains and in some cases DsA2 is expressed instead of DsA1 or the reverse, so for example, DsA2-induced antibodies bind less well to the strains expressing more DsA1 (e.g. FIG. 9, FIG. 16A, 16C, 16D), and DsA1 induced antibodies bind less to the strains expressing more surface epitopes of DsA2 (e.g. FIG. 16A, 16B, 16E). For this reason, e.g. using a hybrid construct, such as hybrid H4, helps to maintain a constant and a balanced level of antibody binding to a large number of strains, irrespective of whether DsA1 or DsA2 is more expressed (e.g. 16 A-F).

Furthermore, the biochemical properties of hybrid polypeptides, such as H4, are even better and more suitable to manufacturing and vaccine administration, than for the full-length DsA1 and DsA2 proteins alone. Protein H4 revealed an increased stability when compared to both full length proteins DsA1 and DsA2, is expressed as a single-band protein in comparison to protein DsA2 and in contrast to DsA1 is resistant to proteolytic degradation (e.g. FIG. 10A, FIG. 10B).

Hybrid polypeptides, such as H4, are therefore not only immunologically more consistent and simpler molecules that can exert the effect of both full-length proteins, but they generally have also an improved stability and purity profile in comparison to its parent versions.

Rationale for a Vaccine Design that Includes Protein PTIP and Hybrid H4 The combination of protein PITP and a DsA1/DsA2 hybrid molecule, such as hybrid H4, enables to target all P. acnes strains related to acne vulgaris pathology (e.g. FIG. 11A-C). A vaccine comprising PITP and a DsA1l/DsA2 hybrid molecule, such as hybrid H4, induces immune responses which demonstrate clear superiority with respect to cross reactivity and cross-type-reactivity even over immune responses induced after vaccination with either of the single proteins alone (e.g. FIG. 9, FIG. 16A-E).

The use of the DsA1/DsA2 hybrid molecule, such as hybrid H4, instead of DsA1 and DsA2 has the clear additional advantage that only one protein is needed instead of two, which facilitates the production process. This becomes additionally important when one additional antigen PITP is included in the vaccine, as it significantly simplifies the production process and reduces the costs.

### Relevance of Functional Assays for Human Subjects

The importance of the functional assays has been shown and developed in the course of the present invention. These functional assays were essential to determine the vaccine antigen composition (especially to select the three proteins DsA1, DsA2 and PITP as vaccine candidates) and to optimize the vaccine design, especially for providing an efficient cross-type-reactive vaccine for administration to human individuals having or being at risk of a pathologically relevant P. acnes infection.

All human subjects contain pre-existing antibodies against P. acnes which can be detected in their bodily fluids such as serum. These antibodies target the antigens expressed by live P. acnes and if the epitopes of these antigens are accessible on the P. acnes cell surface, they are able to induce opsonophagocytic killing of the bacterium (e.g. FIG. 13A, 13B). In fact, according to the data obtained for the present invention opsonophagocytic killing is the most significant functional activity of the surface-binding antibodies, as evidenced by the significant increase in OPK K50 titer in relation to the increase of the surface binding of the human antibodies against P. acnes. Moreover, the efficiency of the opsonophagocytic killing is influenced not only by the amount of the antibody that binds P. acnes, but also by the specific antigenic epitopes expressed on the surface and targeted by the antibodies generated by each individual against the particular P. acnes strain, by the avidity of the polyclonal antibodies towards the specific antigen and the prevalent isotype and subtype of the antibodies generated in the human host during its immune interaction and response against the bacterium.

The Need to Make a Vaccine which Induces Cross-Reactive/Cross-Type-Reactive Antibodies

The present invention provides the answer to the question that currently poses a challenge in the prior art, and that is: “Which bacterial phylotypes should be targeted by a vaccine to provide the most benefit to the host?”

Current status in the prior art is moving completely away from a product that would target a large % of P. acnes strains, because only a small number of phylotypes is considered pathogenic and there is a fear that by inducing immune responses against a larger percentage of P. acnes, this would lead to a complete elimination of P. acnes and pose a threat to humans.

Different phylotypes have been studied in terms of frequency of their isolation from patients compared to healthy individuals and based on the conclusions from these and other studies, the current instruction in the prior art is that any therapeutic product against P. acnes-induced pathologies, such as acne, should act only against the strains of the specific phylogroup considered ‘pathogenic’—and the current status in the art teaches that only the strains of MLSTs type IA1 should be targeted (McLaughlin et al. 2019—page 23; O'Neill and Gallo 2018—page 4, second column, end of the first long paragraph), as these are most commonly isolated from the skin of acne patients, whereas others can be found on both acne-prone and healthy skin. However, this current state of the art knowledge does not take into account that the human host immune system plays important role in regulating the colonization density, virulent behaviour and phylogenetic profile of the strains which colonize different individuals and their bodily areas and that the outcome of these interactions is host-specific. Additional factors, such as hormonal activity, skin barrier integrity and the environment within the sites where infection develops, are also unique to each individual and can contribute to the factors promoting vs limiting the virulent potential of different strains.

The data according to the present invention show however, that not only IA1, but also other phylotypes are equally able to induce acne, since the direct isolation and analysis of the material from inflamed acne lesions (pustules) has revealed that in many patients it is not IA1, but other phylotypes that are predominantly found in this material (e.g. FIG. 14A).

In addition, data according to the present invention show that although skin surface and skin pores of acne patients are colonized by IA1 phylotypes, the patients do not develop acne lesions mostly because of IA1 strains, because acne lesions were also abundant in P. acnes phylotypes different from IA1, and in some cases phylotypes other than IA1 were identified in much higher amount. For example, the P. acnes strains isolated from the inflamed lesion of a specific patient (CR086) have been typed according to SLST scheme and found to be F4, which belongs to IA2 phylotype (e.g. FIG. 14A); whereas the strains detected in the material collected from the skin pores within the same facial area of the same patient included IA1, IA2 and IC phylotypes and in addition one phylotype that was not yet included in the public SLST database (so the MLST classification could not be assigned to this particular strain) (e.g. FIG. 14B). Similarly, the most abundant strains detected in the inflamed acne lesion of the patient CR078 were of the SLS type G1 (phylotype IC) and K1 (phylotype II) (e.g. FIG. 14A), although several strains colonizing the skin pores of this patient included SLS types A1, C1 and C2 (phylotype A1) (e.g. FIG. 14B). This further confirms the need addressed by the objects of the present invention to provide cross-reactive and cross-type reactive vaccines to combat P. acnes infections.

The appearance and the size of the lesions typically vary on the skin, some are smaller, some bigger, some more inflamed some less inflamed. These differences are typically ascribed to different stages of acne development or environment occurring on the skin; however, the data obtained with the present invention show that not only the type of strain isolated from the skin surface or skin pores but also the phylotype composition of the strains that predominate inside the inflamed lesions and the amount and specificity of the antibodies that reach the skin and pilosebaceous follicles of each individual, play crucial role in determining the outcome of the host:bacterium interaction and influence the extent to which an acne lesion develops to become visible on the skin surface leading to clinical symptoms. Therefore, the knowledge about the amount of antibodies, their antigen specificity and effectiveness in inducing phagocytic killing of the various P. acnes strains that colonize the skin of different individuals, has to be taken into account when designing immunotherapeutic products and selecting antigens to be included in a vaccine.

Novelty Over the Prior Art in Terms of Clinical Significance of P. acnes Strains

Studies in the prior art sample the material for the analysis from a single location on the skin which often does not represent a typical location of the acne lesions. For example, the Fitz-Gibbon study (Fitz-Gibbon et al. 2013) has received some negative reviews from the experts in the field due to the fact that they collected the material only from the skin pores on the skin covering the nose, which is not typically affected by acne and because the methodology they used was not considered according to acceptable standards (Eady and Layton 2013: Alexeyev and Zouboulis, 2013). The findings according to the present invention show that it is necessary not only to sample skin pores of the affected and non-affected areas, but also the inflamed lesions, because the strains found on the skin surface or in the pores may not be necessarily the same strains that continue to persist in the inflamed lesions. Only a subpopulation of the strains colonizing each individual has a pathogenic potential in that particular individual, so the strain identity cannot be universally extrapolated by combining the results from different studies and looking for the most common strain isolated from many different individuals. Rather, the analysis and comparison of both bacterium-specific and host-specific factors operating in each particular patient needs to be performed instead.

Therefore, this invention demonstrates also that the data obtained by the analysis of many different individuals cannot be grouped and analysed together to make a conclusion about the relevance of the findings for a single individual whose acne may be due to a phylotype that may not be the most prevalent among the ones identified in other studies, but nevertheless is a pathogenic factor in a particular individual. Instead of evaluating the pathogenic potential of different strains by analyzing the frequencies of their isolation across many different individuals, individual-specific studies in parallel with the analysis of the immune status are much more beneficial, because each individual carries a specific signature of colonizing strains only some of which may be pathogenic in the particular human host. The data provided with the present invention also provides evidence for the differential roles of the specific P. acnes phylotypes in the pathogenesis of acne vulgaris, which is individual-specific. This provides important evidence for a vaccine that is broadly cross-protective, and not only directed to a specific phylotype identified as most frequent in the studies which include many different patients.

Moreover, different sampling areas can be also colonized by different strains, because different strains can get attracted to different environment in the skin. As demonstrated in the course of this invention, in some patients similar strain composition appear in samples collected from skin pores from the forehead and from the cheek, whereas in others significant differences were discovered.

Without having information about the strains found inside the inflamed lesions and the immune status (antibodies and their specificity against the strains identified in the lesion), it is impossible to ascertain the actual relevance of the strains for the acne pathology in each individual. For example, the skin pores of an individual patient (CR078) were colonized by SLST types A1, C1, C2 (MLST phylotype IA1), as well as by SLS types G1 (phylotype IC) and K1 (phylotype U1), however the strains of SLS types G1 (phylotype IC) and K1 (phylotype II) were detected as the most prevalent inside the inflammed acne lesion of the same patient. Similarly, the SLS type F4 (phylotype IA2) was identified as the most prevalent in the inflamed acne lesion of the patient CR086, although at least two additional phylotypes were identified in the skin pores surrounding the same sampled areas on the skin (e.g. FIG. 14B).

### Supportive Evidence for the Selection of Hybrid Molecules of the H4 Kind for Use in a Human Vaccine.

H4 contains functionally relevant epitopes of both Dsa1 and DsA2. There seems to be a tendency for H4-specific antibodies (i.e. antibodies that are induced by a hybrid molecule containing at least one (relevant) DsA1 and one (relevant) DsA2 epitope) to be more abundant compared to DsA1 and DsA2 in severe acne patients. This shows that the patients' immune responses in the skin are driven towards the functionally relevant epitopes of both proteins which are well represented by a hybrid antigen, such as the H4 construct (e.g. FIG. 15).

With the present invention the result of the comparison of a large number of suggested potential candidates and epitopes is provided using a series of functional assays, and the key components (immunologically relevant antigens and epitopes) that must be included in a protective vaccine product are selected. In addition, this invention provides a new direction in the field by revealing the actual relevance of the expression of different virulent factors for the immune response of the human host, which leads to a conclusion that a vaccine product should be designed so to induce immune response against not only a specific phylogenetic group, but against as many as possible, preferably against the most relevant, especially against all bacterial strains which are capable of expressing the virulent factors and obtaining virulent traits and which can be effectively targeted by antibodies raised by immunization of the host.

Human subjects already have an established and ongoing immune interaction with P. acnes and in the course of these interactions they respond differently to P. acnes antigens. The reaction of the immune system to specific antigens (quality of immune response and the antibody amount) vary individually depending on the location and the context in which the specific antigen is encountered. E.g. an antigen may be encountered in tissue secretions, as a part of a damaged or dying bacterial cells, in different areas colonized by P. acnes (not only the skin), in the context of inflammation vs healthy status/tolerogenic state (e.g. normal state of hair follicle vs inflamed, normal vs damaged skin barrier, etc.), or in contact with bacterial biofilms vs planktonic cells. Therefore, intracellular, secreted or membrane-bound proteins are all able to induce an immune response and elicit antibody generation, however, only some of these proteins have a potential to induce a protective immune response which acts against the bacterium.

Therefore, with the present invention, the immunologically relevant antigens and epitopes that elicit most protective responses against the bacterium and that are most suitable for use as a vaccine material (e.g. optimized for manufacturing, formulation and human use) were selected.

### 5.1 Definitions and Description of the Preferred Embodiments

The use of the terms “a” and “an” and “the” and similar referents in the context of describing the invention (especially in the context of the following claims and embodiments) are to be construed to cover both the singular and the plural, unless otherwise indicated herein or clearly contradicted by context.

The terms “comprising,” “having,” “including.” and “containing” are to be construed as open-ended terms (i.e., meaning “including”) unless otherwise noted. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

The term “adjuvant” refers to any substance that when administered in conjunction with an antigen or epitope augments and/or redirects the immune response to the antigen or epitope. Adjuvants can augment an immune response by several mechanisms including lymphocyte recruitment, stimulation of B and/or T cells, and stimulation of macrophages. The adjuvant may be bound to the antigen by covalent binding, electrostatic interaction or adsorption. Antigen/epitope and adjuvant activities may be combined by genetically (recombinantly) fusing coding regions, or portions thereof, of antigen and adjuvant; thus, the immunogen may contain only one ingredient or component. The adjuvants may be artificial or naturally occurring.

In the present context, adjuvants include emulsion-based adjuvants, aluminium hydroxide gel, solid phase adsorbents, nanospheres and encapsulating materials such as liposomes. The vaccine as described herein particularly employs a “human adjuvant formulation”, which is understood to be specifically compatible with the human immune system. A human vaccine formulation particularly does not contain Freund's incomplete adjuvant (and, of course, not Freund's complete adjuvant or similar formulations), which is often used only for producing animal immune sera. Contrary to non-human adjuvant formulations, such as those used to produce rabbit immune sera, the human adjuvant formulations are particularly characterized by the use of an adjuvant licenced for human use to assure a high safety level (not inducing undesirable local or systemic effects), and no immune responses against the human host itself while promoting the required immune response in the vaccine target population via an optimal vaccine administration route.

Exemplary adjuvants are metal salts (e.g., aluminium or calcium salts), high molecular weight molecules, cationic peptides, CpG oligonucleotide, squalene based adjuvants (e.g. MF59). Metal salts include alum (potassium aluminum sulfate), aluminum hydroxide, aluminum phosphate, aluminum oxohydroxide, aluminum hydroxyphosphate, calcium phosphate, cerium nitrate, zinc sulfate, colloidal iron hydroxide, and calcium chloride. Several aluminum adjuvants with different physical properties are commercially available and approved for human use.

In the present context, the adjuvant may also be any suitable, high molecular-weight molecule, typically a protein or large (i.e., generally greater than 6000 kD) molecule of sufficient molecular complexity to elicit an immune response for an antigen or epitope that is covalently linked to it. The category of suitable high molecular weight adjuvants is exemplified by toxins, toxoids or any mutant cross-reactive material of the toxin from tetanus, diphtheria, pertussis, Pseudomonas species, E. coli, Staphylococcus species, and Streptococcus species. Such toxins or toxoids may be tetanus toxoid, pertussis toxoid, cholera toxoid, E. coli LT, E. coli ST, and exotoxin A from Pseudomonas aeruginosa; bacterial outer membrane proteins such as outer membrane protein complex c (OMPC), porins, transferrin binding proteins, pneumolysin, pneumococcal surface protein A (PspA), pneumococcal adhesin protein (PsaA), C. difficile enterotoxin (toxin A) and cytotoxin (toxin B) or Haemophilus influenzae protein D, other pharmaceutically acceptable polypeptide carriers, such as ovalbumin, keyhole limpet hemocyanin (KLH), bovine serum albumin (BSA) or purified protein derivative of tuberculin (PPD); cationic peptides, CpG oligonucleotide, squalene-based adjuvants, preferably MF59; cytokines, such as IL-1 and IL-2; or combinations thereof. Alternatively, the polypeptides according to the present invention may also be formulated as virus-like particles, bound to (monoclonal) antibodies or nanoparticles (recently reviewed in Malonis et al., Chem. Rev. 120 (2020), 3210-3229). The latter are specifically useable for conformational epitopes. Conformational antibodies, such as R87-K90+S234-G250, R87-K90+L246-A260, R87-K90+A256-E270, R87-K90+R266-T277, A310-D313+V289-K296, A310-D313+V289-K296. A310-D313+T285-T300, A310-D313+A144-N157, A310-D313+T285-R286, A310-D313+T285-D290, A310-D313+T293-E307 of DsA can either be presented essentially by their native environment as DsA1 polypeptide, especially as DsA1 fragment or DsA1 derivative according to the present invention. Alternatively, these conformational epitopes may also be presented as artificial scaffold. Estabished techniques for such scaffold presentation are, as just stated, virus-like particles, bound to (monoclonal) antibodies, nanoparticles, alphabodies, protein A, protein G, designed ankyrin-repeat domains (DARPins), tibronectin type III repeats, anticalins, knottins, or engineered CH2 domains (nanoantibodies) (see e.g. Malonis et al., 2020, US 2019/0383829 A1, WO 2019/123262 A1).

In certain embodiments, the vaccine as described herein comprises an adjuvant which is a heterologous chemical or biological material or substance which is commonly used to enhance the active immune response following vaccination with a vaccine antigen. Typically, an adjuvant would be alum, e.g. as phosphate or hydroxide, TLR agonists, such as CpG or monophosphoryl lipid A. Cytokines such as IL-1 and IL-2 can be also used as adjuvants.

The term “heterologous” or “exogeneous” with respect to an adjuvant as used herein refers to a molecule derived from a source other than the P. acnes antigen. A heterologous adjuvant may refer to an artificial, inorganic or organic compound or substance, and is optionally an adjuvant derived from a different bacterium or a different P. acnes strain or subtype or from an unrelated source (e.g., a different pathogen, chemical synthesis or an organic or inorganic material).

The selected vaccine antigen can be used also in a non-adjuvanted form and presented in a physiological solution or a formulation suitable for skin-specific immunization. An intradermal, transdermal or a subcutaneous administration could be performed using a variety of methods, including intradermal injection applicators, microneedles, transdermal laser devices, skin patch or other suitable skin-adapted application procedures (Engelke et al. 2015).

The term “polypeptide” as used herein, refers to both, larger polypeptides (proteins), e.g. DsA1, DsA2 and PITP and larger fragments and derivatives thereof, and shorter polypeptides (oligopeptides), e.g. the epitopes, fragments or derivatives of DsA1, DsA2 and PITP.

The term “antigen” as used herein shall refer to a whole molecule or a fragment of such molecule, either within the natural environment or as isolated antigen, which also encompasses recombinant antigens or a single antigen produced by genetic engineering of a host cell transformed with a recombinant heterologous nucleotide sequence, which antigen is specifically bound by an antibody binding site. Specifically, the term encompasses also substructures of an antigen, e.g. involving a polypeptide and/or carbohydrate structure, generally referred to as “epitopes”, e.g. B-cell epitope or T-cell epitope, preferably B-cell epitopes, which are immunologically relevant. An antigen may be an immunogen as such, or in the case of low immunogenicity, become an immunogen upon suitable engineering or formulation. As used herein, the terms “immunogens” or “antigens” also encompass epitopes, and are used interchangeably.

The term “epitope” as used herein shall in particular refer to a molecular structure which may completely make up a specific binding partner or be part of a specific binding partner to a binding site of an antibody or a cognate T cell receptor. An epitope may either be composed of a carbohydrate, a peptidic structure, a fatty acid, an organic, biochemical or inorganic substance or derivatives thereof, and any combinations thereof. If an epitope is comprised in a peptidic structure, such as a peptide, a polypeptide or a protein, it will usually include at least 6 amino acids, preferably at least 7, 8, 9, or 10, up to 40 amino acids, and more preferably from 6 to 35, from 7 to 30, from 8 to 25, especially from 10 to 20 amino acids. Epitopes can be either linear or conformational epitopes. A linear epitope is comprised of a single segment of a primary sequence of a polypeptide or carbohydrate chain. Linear epitopes can be contiguous or overlapping. Conformational epitopes are comprised of amino acids or carbohydrates brought together by folding the polypeptide to form a tertiary structure and the amino acids are not necessarily adjacent to one another in the linear sequence.

Epitopes of a given antigen can be identified using any number of epitope mapping techniques, well known in the art. See, e.g., Epitope Mapping Protocols in Methods in Molecular Biology (Morris 2005). For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871. Alternatively, instead of epitopes (either linear, conformational or both), mimotopes (either linear, conformational or both) can be used in the vaccines according to the present invention, which mimic the structure of an epitope and induce the immune response similar to the one elicited by the epitope. An antibody for a given epitope antigen will recognize a mimotope which mimics that epitope. Mimotopes are commonly obtained from phage display libraries through biopanning. Mimotope analysis has been widely used in mapping epitopes (Smith and Petrenko, 1997), developing therapeutics (Macdougall et al. 2009) and vaccines (Knittelfelder et al. 2009). A “mimotope” is a polypeptide, which differs from the polypeptides disclosed herein by one or more amino acids but which mimics the three-dimensional structure of a wild-type polypeptide/epitope. A mimotope generally in the context of a larger protein backbone called carrier is able to stimulate a host's immune system to produce an antibody antigen-specific response. The host generates antibodies that specifically bind to the mimotope and the corresponding wild-type epitopes disclosed herein. A mimotope may have a primary amino acid sequence capable of eliciting a T-cell effector response and/or a three-dimensional structure necessary to bind B-cells resulting in maturation of an acquired immunological response in an animal, especially also in humans. An antibody for a given epitope antigen will recognize a mimotope which mimics that epitope. “Mimotopes” are therefore polypeptides mimicking protein, carbohydrates or lipid epitopes and can e.g. be generated by phage display technology. Coupled to carriers or presented in a multiple antigenic peptide form mimotopes achieve immunogenicity and induce epitope-specific antibody responses upon vaccination. Mimotopes can be polypeptides, such as peptides with an amino acid sequence length of at least about 8 to about 25 amino acids or more.

Exemplary algorithms and programs based on mimotope-based prediction models include, e.g., MimoPro (http://informatics.nenu.edu.cn/MimoPro), PepSurf (http://pepitope.tau.ac.il and EpiSearch (http://curie.utmb.edu/episearch.html). Further, sequence-based prediction models are available which only rely on the primary sequence of an antigen, e.g. BEST and Zhang's method as reviewed in Sun et al. 2013. In addition, binding sites prediction models can be used which infer methods that that focus on binding sites prediction of protein-protein interaction the interaction of an antigen and an antibody, e.g. ProMate, ConSurf, PINUP, and PIER.

Similarly, conformational epitopes may be identified by determining spatial conformation of amino acids such as by, e.g., x-ray crystallography and 2-dimensional nuclear magnetic resonance. See. e.g., Epitope Mapping Protocols, supra.

The term “immunologically relevant” with respect to an antigen or epitope as used herein shall refer to its ability to be recognized by the immune system of the recipient organism and induce antibodies in the recipient which are cross-binding and/or cross-reactive, especially cross-type-reactive, and which have antibacterial or pathogenesis ameliorating activity.

Specific antigens as described herein are any of the proteins identified by the UniProt accession numbers Q6A5X9, Q6A5P9 and Q6A9N1 of the P. acnes strain KPA171202, or an analogous protein, in particular a homologue protein of any of the proteins identified by the UniProt accession numbers Q6A5X9, Q6A5P9 and Q6A9N1 which is of a different P. acnes strain. Any reference to sequence databases herein shall—in case of doubt—refer to the version of the database at the priority date of the present invention. This is also reflected in the present specification, e.g. in FIG. 12 or in the sequence listing.

Proteins Q6A5X9, Q6A5P9 and Q6A9N1 are also referred to in the present invention as examples of native “DsA1”, “DsA2” and “PITP” polypeptides, especially in the example section (as preferred examples (“P022”, “P027” and “P028”) and in the figures. As also further outlined below, according to the present invention, a “DsA1 polypeptide”, “DsA2 polypeptide” and “PITP polypeptide” includes a native and functional polypeptide of a P. acnes strain comprising all regions and domains as defined below. A “DsA1 polypeptide”, “DsA2 polypeptide” and “PITP polypeptide” further includes fragments and derivatives of DsA1, DsA2 and PITP, respectively. A “DsA1 fragment”, “DsA2 fragment” and “PITP fragment” refers to a fragment of a native and functional DsA1 polypeptide, DsA2 polypeptide, or PITP polypeptide, respectively of a P. acnes strain comprising a certain minimum amino acid length with at least 7, preferably at least 8, more preferred at least 9, especially at least 10 consecutive amino acids, of a DsA1 polypeptide, DsA2 polypeptide, or PITP polypeptide, respectively (i.e. a fragment with a consecutive native polypeptide sequence of these functional P. acnes polypeptides). Preferably, a “DsA1 fragment”, “DsA2 fragment” and “PITP fragment” comprises at least an epitope of the DsA1 polypeptide, DsA2 polypeptide, or PITP polypeptide, respectively, which can be recognized by the human immune system and elicit functional anti-P. acnes antibodies reactive against this epitope (i.e. also being efficient in the opsonization assay as disclosed herein or otherwise efficient in promoting a pathology ameliorating effect, i.e. via T-cells (without suitable antibodies), or by antibodies removing/inactivating secreted forms of the polypeptide). A “DsA1 fragment”, “DsA2 fragment” and “PITP fragment” includes also deletions of native polypeptides (i.e. being composed of two or more fragments of the native polypeptides. A prominent example of typical fragments are fragments comprising the N-terminal methionine but lacking a portion of the N-terminal part of the polypeptide after the N-terminal methionine. According the definitions herein, a fragment which only lacks the signal peptide (but which may include the N-terminal methionine) is regarded as a (full and native) DsA1/DsA2/PITP polypeptide. Fragments which comprise additional deletions, e.g. N-terminally in the NSR of DsA1/DsA2 and in the ENFD of PITP but which still contain the N-terminal methionine are referred to herein as “fragments”. N-terminal extensions resulting from DNA-level frame-shifts and replacement of a signal peptide by alternative-frame frame sequences are equally treated as falling under the definition of native polypeptides of fragments herein, because the actual polypeptides are “functional” within the meaning of the present invention. A “DsA1 derivative”, “DsA2 derivative” and “PITP derivative” refers to a polypeptide being derived from a DsA1 polypeptide, DsA2 polypeptide, or PITP polypeptide, or DsA1 fragment, DsA2 fragment, or PITP fragment (having at least the amino acid residue numbers of the “fragments” according to the present invention (i.e. at least 7 amino acids) and comprises at least one amino acid exchange/deletion/insertion compared to a native DsA1/DsA2/PITP sequence (i.e. polypeptide/fragment) but—neverthesless—also comprising at least an epitope which can be recognized by the human immune system and elicit functional anti-P. acnes antibodies reactive against this DsA1 polypeptide epitope. DsA2 polypeptide epitope, or PITP polypeptide epitope, respectively, (i.e. also being efficient in the opsonization assay as disclosed herein). A “derivative” as used herein can be derived from one or more of DsA1, Dsa2 and PITP, i.e. a polypeptide comprising a DsA1 and a DsA2 epitope may be referred to as “DsA1 derivative” as well as a “DsA2 derivative”. Preferred derivatives according to the present invention contain at least an epitope of a DsA1 polypeptide, DsA2 polypeptide, or PITP polypeptide, i.e. an epitope which is identical in amino acid sequence as in the native (wild type) P. acnes DsA1/DsA2/PITP polypeptide. As also defined below, an epitope as described herein is a polypeptide sequence which is able—upon administration to human individuals—to elicit antibodies with antibacterial activity, as determined in a functional assay (preferably the opsonization assay as disclosed below, especially the opsonization assay as disclosed in the example section). Even more preferred derivatives are combinations comprising one or more DsA1/DsA2/PITP fragments (i.e. one or more native polypeptide fragments of these P. acnes proteins).

The protein identified by the UniProt accession number Q6A5P9 is specifically characterized by the following characteristics: hypothetical and uncharacterized protein, (locus tag=PPA2210). The protein has been found by Holland et al., to be upregulated during stationary phase (Holland et al. 2010) and to be variably expressed among P. acnes strains by different genetic phylotypes (Lodes et al. 2006; Yu et al. 2016; McLaughlin et al. 2019).

The protein identified by the UniProt accession number Q6A5X9 is specifically characterized by the following characteristics: putative adhesion or S-layer protein, (locus tag=PPA2127), with a N-terminal sequence in place of a signal peptide at amino-acids 1-28 and the mature polypeptide chain including amino acids 29-405. The N-terminal sequence is though to be the effect of a genomic frame-shift event and may not be part of the mature protein. The expression of this protein was found to be highly variable between P. acnes isolates, using the genetic and proteomic analysis techniques (Lodes et al. 2006; Brzuszkiewicz et al. 2011; Yu et al. 2016).

The Q6A5X9 (PA-25957) has been described by Lodes et. al. (Lodes et al. 2006) as being weakly similar to M-like proteins found in other bacterial species, to have a common cleavable signal sequence, a hydrophilic proline rich repeat near the carboxy-terminus and a LPXTG motif and to contain transmembrane helices in their mid-regions. M-like proteins in general were found to interact with the complement system and binding various forms of glycosaminoglycans including dermatan sulphate. The Streptococcal M proteins are known to be more conserved towards the C-terminus wherein a cross-reactive epitope has been identified: antibodies recognizing the C-terminal part of this protein cross-reacted with 30 different serotype strains (Fischetti 1989). Lodes et al. have also suggested that the C-terminus of PA-25957, later named DsA1 (McDowell et al. 2011), which contains PT repeats is highly antigenic and associated with ‘health’, since higher reactivity of ‘acne negative’ sera was detected against this region compared to the rest of the P022 sequence. The PT region was also found to be more variable and suggested to additionally contribute to the differences in the expression on different strains.

In addition, Lodes et al. suggested that a cleavable LP(X)TG domain identified in the sequences of both PA-25957 (Q6A5X9) and PA-5541 (Q6A5P9) is involved in membrane anchoring and is missing in some P. acnes isolates; suggesting that PA-25957 DsA1 is not only found on the cell membrane, but is also secreted by some strains and that this further contributes to the variation in the expression of these proteins.

According to the prior art, these proteins are expressed mostly by Type IA strains, which are predominantly isolated from acne-affected skin (Bek-Thomsen et al. 2014; McLaughlin et al. 2019), however, since no specific protein expression signature could be detected in the sebaceous hair follicles of acne-prone compared to healthy skin (Bek-Thomsen et al. 2014), and the study also had many methodological limitations described in the publication, this left many questions open in terms of their actual significance in the disease pathology and the relevance for use in acne treatment. For this reason, P022 (DsA1) and P027 (DsA2) are currently considered as virulence factors but not vaccine candidates (O'Neill and Gallo 2018). Moreover, the prior art as of today is very cautious about employing a vaccine-based approach and is moving away from it in the favor of strategies that do not directly act against P. acnes but target secreted factors (Contassot 2018; Keshari et al. 2019) or use alternative therapies, such as probiotics (O'Neill and Gallo 2018; Bruggemann 2019. McLaughlin et al. 2019 (“We can speculate that the potential for type IAs to modulate their interaction with the host immune system via the DsA immunogenic proteins may be important in the recurring nature of acne”); which is a very vague statement but is consistent with the data available at the time of the present invention)). In fact, McLaughlin et al. (2019) did not even refer only to these antigens: in this paper it was shown that Type I-A expresses a multitude of different virulence factors in addition to DsA proteins and concluded that systematic studies are lacking in the field to compare the potential of all of them to each other and decide on which ones to choose. Furthermore, Keshari et al. (2019) emphasizes that among different virulence factors expressed by P. acnes, the vaccine should include only those that are secreted and not cell surface-associated, cautioning again that P. acnes is a commensal bacterium and that the bacterium itself should not be targeted by a vaccine.

Yu et al. (Yu et al. 2015; Yu et al. 2016) has employed proteomic analysis to analyze the expression of different proteins by acne-associated strains and found that among other proteins studied, the expression of the proteins corresponding to Q6A5X9 (gi 50843565) and Q6A5P9 (gi 50843645) was highly variable among P. acnes strains from different genetic groups and phylotypes and that Q6A5P9 was absent or much less expressed by the P. acnes ribotypes considered ‘virulent’.

According to a preferred embodiment of the present invention, modified P022 (DsA1) and/or P027 (DsA2) polypeptides are provided (DsA1 fragments, DsA1 derivatives, DsA2 fragments and DsA2 derivatives) which have advantageous properties compared to wild type P022 and P027 proteins from P. acnes and are specifically suitable for vaccination purposes. The novel use of DsA1/DsA2 proteins (e.g. P022 and P027) according to the present invention for being used to interfere with (i.e. prevent and/or treat pathological conditions caused by) P. acnes is based on their advantageous properties (as revealed by the present invention), both with respect to their immunogenic properties as well as with respect to their handling properties (which enable easier large-scale recombinant expression and production). Both advantages appeared in the course of generation of the present invention and are surprising in view of the knowledge in the art.

According to a preferred embodiment, the present invention also relates to fragments or derivatives of DsA1/DsA2 polypeptides. A DsA1/DsA2 “fragment” is a part of a naturally occurring DsA1 or DsA2 protein; a DsA1/DsA2 “derivative” is a non-natively occurring polypeptide which comprises a DsA1/DsA2 fragment which contains at least an antigenic epitope (i.e. an epitope which is immunogenic and accessible to antibody binding on the surface of P. acnes). DsA1/DsA2 fragments or derivatives preferably have a length of at least 15, preferably at least 20 amino acids, even more preferred at least 30 amino acids, especially at least 50 amino acids of a naturally occurring DsA1 or DsA2 protein. A preferred DsA1 or DsA2 fragment or derivative according to the present invention has a shortened PT repeat region, preferably having only one, two, three, four or five PT repeats. Although the PT repeat region has been regarded as being “highly antigenic” in the prior art (Lodes et al., 2006) and was therefore seen as immunogenic in principle, evaluation of this protein region in the course of the present invention by using patient antibodies showed that this region turned out to be not essential for providing suitable acne vaccines. More specifically, DsA1 or DsA2 polypeptides lacking most of the PT repeat region surprisingly show significant immunogenic advantages compared to DsA1 or DsA2 polypeptides wherein one or only a few PT repeats are present. Moreover, DsA1 or DsA2 peptides with fewer PT repeats induced antibodies with stronger cross-reactivity/cross-type-reactivity compared to DsA1 or DsA2 peptides containing the complete (wild type) PT repeat region. It is even possible to omit the whole PT sequence; however, preferred embodiments of the present invention contain at least one PT repeat.

In connection with the terminology used herein, all references to sequences, fragments, etc., herein always refer to consecutive amino acids (unless explicitly referred to the contrary). For example, a DsA1/DsA2 fragment of at least 15 amino acids always refers to at least 15 consecutive amino acid residues of a DsA1/DsA2 polypeptide. The term “consecutive” means that the given amino acid is at the given position in the alignment of FIG. 12. Interchanges between different DsA1/DsA2 homologues are, of course, to be included, if the position is kept (i.e. no deletions, unless explicitly foreseen in FIG. 12).

For example, UniProt amino acid sequence Q6A5X9 refers to the protein “PPA2127” of P. acnes strain DSM 16379/KPA171202, i.e. to the polypeptide with the following sequence:

MLSLLRRLLR WSDQSQQSPP PPPPQAEASS NRPRSVAQAA
IATDGKGIID KDCRDAVIND AKLRAAIAGA LVKAGFSSAD
AVALAPRIAK EMAKEGVLLI NHHKLKALIG AQLGLLTDAK
IQRAAAAVDL GIKATLAATI IPNALHSAAF KDAVVANLVA
AGVDKKLAKA TAVAIAATAL NPALGPIAKT EAIKAEIAAQ
AALLVGRGVH LKKAAIEHII GRSFDAAVAT AIVSSPILNA
RIVTHLVRAG IDKSLAVQIA PRIIDRLAKE PLLALNTAKL
MKNITRQIVD VITADKAIKT AEQLEKELPA LDDLVKKACS
CPKPTPTPTP TPTPTPKPTP TPTPKPTPTP KPKPTPAPAP
TSGATSDEST SRSGGHSQGG SGTHYIHHGV APVLTHSSDL
PSTGF

In this wild type DsA1 polypeptide, the PT repeat region extends from proline 324 (P324) to threonine 361 (T361) and contains 19 PT repeats. UniProt amino acid sequence Q6A5P9 refers to the protein “PPA2210” of P. acnes strain DSM 16379/KPA171202. In this wild type DsA2 polypeptide, the PT repeat region extends from proline 367 (P367) to threonine 420 (T420) and contains 27 PT repeats. Within the meaning of the present invention, the term “PT repeat” is defined as a section in the primary amino acid sequence of the DsA1/DsA2 protein with repetitive proline containing dipeptides. It usually follows a consensus sequence in DsA1 (and also in DsA2) of P. acnes DX₁LVX₂KACX₃(C)PX₄ (wherein X₁ is usually D (in DsA1 or G (in DsA2); X₂ is usually K (in DsA1) or Q (in DsA2); X₃ is usually S (in DsA1) or T (in DsA2); and X₄ is usually K (in DsA1) or E or D (in DsA2)). By this definition according to the present invention, the initial “PK” (in DsA1) or “PE” or “PD” (in DsA2) dipeptide stretch sequence is not defined as part of the PT repeat region which starts with the first proline residue after this “PK”, “PE” or “PD” dipeptide. The proline containing dipeptides in the PT repeat regions are mainly proline-threonine (PT) dipeptides but may also be proline-alanine (PA), proline-asparagine (PN), or proline-lysine (PK) dipeptides; however, the PT repeat regions mainly consist of PT dipeptides. As an alternative to a series of PT dipeptides polar/acidic/negatively charged peptides such as “SDTDTDSNPNADADTDA” can be found. Those polar/acidic/negatively charged peptides are composed of “SD” and “TD” dipeptides but also “SN”, “PN” or “AD” and less frequently “AP” are possible. For example, the PT repeat region in Q6A5X9 consists of 19 repetitive proline dipeptide stretches with 13 PT, four PK, and two PA dipeptides; the PT repeat region in Q6A5P9 consists of 27 repetitive proline dipeptide stretches with 22 PT and five PA dipeptides. Optionally the PT repeat region can also be glycosylated A “DsA1 polypeptide of P. acnes” according to the present invention is a naturally occurring DsA1 protein of a P. acnes strain (“native DsA1”), comprising all of the domains (from N- to C-terminus): N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Thr repeat containing region (“PT repeat region”), and a C-terminal region (“CTR”; often with an LPXTG motif close to the C-terminus) or, optionally a DsA polypeptide with a shortened PT repeat region. The PT repeat region located between CSD3 and CTR is—in preferred DsA1 polypeptides according to the present invention—either shortened or not present at all (compared to naturally occurring DsA1 proteins). The sequence of naturally occurring DsA1 polypeptides contained in the sequence database (see e.g. FIG. 12) are highly identical and differ (besides point mutations and rare exceptions specifically of the presence of the terminal LPXTG motif) only in the length and composition of the PT repeat region.

The alignment of selected, representative naturally occurring DsA1 variants shows the more or less ubiquitous invariance of the protein. There are three notable exceptions. First, frequently the gene is in fact a pseudogene due to a frame-shift at the C-terminal end of the signal peptide. These genes are not expected to be expressed aside of the signal peptides. In gene prediction these variants turn up with an N-terminal extension, which is not expected to be expressed, however, but is rather an artefact of gene prediction. Strain KPA171202 has been included as a representative example. It may or may not be possible that these frame-shifts are reversible (a form of phase variability regulation) at some specific condition. Second, the length of the PT-repeat varies significantly between isolates. This PT-repeat also appears to be a frequent source of mis-assemblies, creating the impression of deleted C-termini. When considering the entire genomes, however, it appears C-termini are more or less universally conserved, at least in expression competent proteins, and reported variants are most often sequencing artefacts. There is a rare (about 10% of cases) alternative frame-shift mutation removing the last 10 amino-acids of the C-terminus. This is interesting as this variant lacks the C-terminal LPXTG motif assumed to be critical for cell-wall anchoring of the protein. However, this specific frame shift appears to be limited to proteins also frame-shifted in the N-terminus, so which are presumably not expression competent. Therefore, this variant is presumably not produced in-vivo. In any way, this variant is not defined as a “DsA1 polypeptide” within the meaning of the present invention but is defined as a “DsA1 derivative”, since it does not contain all domains/region of DsA1 as defined above. Taken together, this shows that DsA1, while generally thought of as hypervariable protein, is in fact highly conserved. Among existing variabilities only PT-variability may be understood as true protein-variability, specifically because the transient nature of observed genomic frame-shifts has not been determined so far and the effect of N-terminal frame-shifts is essential non-expression. The other exception may be the rare C-terminal loss of the anchorage motif, but that according to current data is either very rare or limited to genes not expressed as proteins (i.e. also “DsA1 derivative” and not “DsA1 polypeptides”, because these sequences do not contain all domains/regions of DsA1 as defined above). These data evidence the DsA1 polypeptides as essentially invariable. Indeed when considering DsA1 sequence variants within regions CSD1-SR1-CSD2-SR2-CSD3 maximum observed difference between sequences has been determined so far as 4-0-4-3-2 amino-acids and dissimilarity (i.e. dissimilar amino-acids) as 1-0-4-2-2. Similarly when considering DsA2 sequence variants within regions CSD1-SR1-CSD2-SR2-CSD3 maximum observed difference between sequences has been determined as 4-1-9-0-4 amino-acids and dissimilarity (i.e. dissimilar amino-acids) as 3-1-6-0-1. DsA1 and DsA2, although clearly homologues, are distinct with a minimum amino-acid difference between CSD1-SR1-CSD2-SR2-CSD3 regions of 25-6-22-1-23 and 15-4—8-1-14 dissimilar residues, respectively.

Importantly, this considerable invariability of DsA1 does not suggest or prioritize any part of the protein over another. Specifically, when carefully considering the assembly errors in public repositories even typical PT-length variability may commonly be exaggerated. Moreover, if anything, the length variability of the PT region could be understood as an effect of immunological pressure and has been interpreted as such in the literature. This was shown to be incorrect by the data provided with the present invention, in fact the present experimental results demonstrate the opposite, PT is immunologically largely irrelevant. With the present data it is clear that this is a linker tethering the N-terminal domain(s) to the cell-surface. This further solidifies that sequence variability does not obviously suggest any specific part of the DsA1 sequence as vaccine candidate and such interpretation (in the case of PT) is in fact incorrect.

Some sequence entries do not contain a CTR; these entries are most likely either (sequencing) artefacts or are not DsA1 polypeptides within the meaning of the present invention, i.e. that they do not function as DsA1 proteins in P. acnes. These sequences are therefore also referred to as “DsA1 derivatives”. The sequence numbering used according to the present invention is based on the numbering of Q6A5X9 which means that e.g. the proline-lysine bipeptide preceding the PT region amino acids is always referred to as “P322” and “K323” even if a DsA1 polypeptide (or fragment or derivative) with a different length (i.e. wherein e.g. P322/K323 is at a different amino acid number in this specific polypeptide) is concerned. The mRNA of DsA1 encodes also a N-terminal signal sequence (beginning with an N-terminal methionine residue and ending with a proline-glutamine-alanine-glutamic acid-alanine sequence) which is not part of the mature polypeptide. Accordingly, the DsA1 polypeptide of P. acnes” according to the present invention begins with the serine residue 29 (S29) of the amino acid sequence Q6A5X9 in the UniProt database and ends with phenylalanine 405 (F405).

Accordingly, the functional domains of the DsA1 polypeptide (as referred to herein) are defined—on the basis of the numbering in Q6A5X9—as follows (see also: e.g. FIGS. 12A and 12B) NSR: from S29 to I48, CSD1 from 149 to L130, SR1 from 0131 to S147, CSD2 from A148 to L267, SR2 from A268 to T277, CSD3 from A278 to K323, the PT repeat region from P324 to T361, and CTR from S362 to F405. The signal peptide (SP) of M1 to A28 is usually not contained in the final polypeptide (and in the vaccine); except the N-terminal methionine, which may be present. Accordingly, an SP is not part of the mature (functional) sequence and not part of the antigen, and not part in a production setup for the vaccine formulations disclosed herein; however, when produced in some form of continuous fermentation (i.e. when not harvesting and breaking open cells), secretion into the medium using a signal peptide may use appropriate SPs).

A “DsA2 polypeptide of P. acnes” according to the present invention is a naturally occurring DsA2 protein of a P. acnes strain (“native DsA2”), comprising all of the domains (from N- to C-terminus): NSR, CSD1, SR1, CSD2, SR2, CSD3, a PT repeat region, and a CTR, as defined above for DsA1. The sequence of naturally occurring DsA2 polypeptides contained in the sequence database (see e.g. FIGS. 12A and 12C) are highly identical and differ (besides point mutations) only in the length and composition of the PT repeat region. Some sequence entries do not contain a NSR, CSD1 and CTR: these entries are most likely either (sequencing) artefacts or pseudo-genes and are not DsA2 polypeptides within the meaning of the present invention, i.e. that they do not function as DsA2 proteins in P. acnes; nevertheless, they have been included in the alignments in the Figures to illustrate possible sequence variations in the reported sequences in the databases.

The term “wherein the amino acid numbering corresponds to the amino acid sequence Q6A5X9 in the UniProt database” means that the numbering in the claims and embodiments usually refers to the numbering of the amino acid sequence of the DsA1 protein. This indicates that the corresponding sequences of DsA2 are meant to correspond with the relevant position in the DsA1 protein, if applicable. This means that the sequence numbering for DsA2 used according to the present invention (unless explicitly stated otherwise) is based on the numbering of Q6A5P9 which means that e.g. the proline-glutamic acid bipeptide preceding the PT region amino acids is always referred to as “P365” and “E366” even if a DsA2 polypeptide (or fragment or derivative) with a different length (i.e. wherein e.g. P365/E366 is at a different amino acid number in this specific polypeptide) is concerned. The mRNA of DsA2 encodes also a N-terminal signal sequence (beginning with an N-terminal methionine residue and ending with a proline-leucine-proline-alanine-asparagine-alanine sequence) which is not part of the mature polypeptide. Accordingly, the DsA2 polypeptide of P. acnes” according to the present invention begins with the alanine residue 72 (A72) of the amino acid sequence Q6A5P9 in the UniProt database and ends with alanine 463 (A463).

Accordingly, the functional domains of the DsA2 polypeptide (as referred to herein) are defined—on the basis of the numbering in and the sequence of Q6A5P9:

MIIFVRVGNR HMRKRRAVLV VAFQLVNRRL AITKEPTPRR
NDASNKNHCG SCDSNCLNRL TISYHPLPAN AASNGNSSIT
QSAAFSPRAT TKISEDCRKA IINDLKLRGA IVGALVKAGL
SAADAAALAP RIAAEMAAEG TLTINHHRLK VLVASQLGLV
ADAAVQHAAA AIDLSFKAIL GASIIPNALG SAAFKNAVIA
NLVAAGIDKH LARATAVAIV ATALNPALGP IAKFELIKAE
IAAQAALLIR RGVHLQKAAI EHVIGRAFDA AVATAIISSP
ILSARIVTHL VRAGIDKSIA ISLAPHIVKR LAKEPLLAFN
TAKLVKDIAR QIVDIRNTQE AIAVYKQLKA ELPTLDGLVQ
KACTPEPTPT PTPTPTPTPT PAPTPTPAPT PTPAPTPAPT
PTPAPTPTPT PTPTPTPTPT HGATTTTPIS RTTDRHNLGS
HHTRIAAPAL IHAKALPATG TGA

—as follows (see also: e.g. FIGS. 12A and 12C) NSR: from A72 to K92, CSD1 from 193 to L174. SR1 from S175 to S191, CSD2 from A192 to L311. SR2 from A312 to T321, CSD3 from A322 to E366, the PT repeat region from P367 to T420, and CTR from H421 to A463. The signal peptide (SP) of M1 to A71 is usually not contained in the final polypeptide (and in the vaccine); except the N-terminal methionine, which may be present. Accordingly, an SP is not part of the mature (functional) sequence and not part of the antigen, and not part in a production setup for the vaccine formulations disclosed herein; however, when produced in some form of continuous fermentation (i.e. when not harvesting and breaking open cells), secretion into the medium using a signal peptide may use appropriate SPs).

In this context it is relevant to recollect that DsA1 and DsA2 proteins are homologues (paralogues) with a typical sequence identity between 60-71%, also depending which region of the protein is aligned. But excluding PT-region length polymorphism a high degree of typically >90% amino-identity within intact DsA1 and DsA2 proteins can be seen, respectively. DsA1 and DsA2 are clearly quite similar. It is therefore important to effectively differentiate these proteins. A sequence is therefore preferentially classified as DsA1 if the local alignment of this sequence with Q6A5X9 (the characteristic DsA1 template) joined regions CSD1-SR1-CSD2-SR2-CSD3 comprises at least 70% of the length of the Q6A5X9 CSD1-SR1-CSD2—SR2-CSD3 region and the amino-acid identity is higher than in the local alignment of the matching new sequence to Q6A5P9 and also comprising at least 70% of the Q6A5X9 CSD1-SR1-CSD2-SR2-CSD3 region.

A sequence is preferentially classified as DsA2 if the local alignment of this sequence with Q6A5P9 (the characteristic DsA2 template) joined regions CSD1-SR1-CSD2-SR2-CSD3 comprises at least 70% of the length of the Q6A5P9 CSD1-SR1-CSD2-SR2-CSD3 region and the amino-acid identity is higher than in the local alignment of the matching new sequence to Q6A5P9 and also comprising at least 70% of the Q6A5X9 CSD1-SR1-CSD2-SR2-CSD3 region.

What has been said about the apparent variability or rather invariability of DsA1 is mostly also true for DsA2, an aspect to be expected considering these are fairly close homologues. However, more DsA2 variants are known than DsA1 variants, which indicates a comparably higher variability given that P. acnes strains normally encode one protein of each type, although in some cases as apparent pseudo-genes. On 6 Nov. 2019, 100 DsA1 variants can be found in the NCBI protein database versus 173 DsA2 variants (the database source for sequences used for the present invention is generally the NCBI protein database (https://www.ncbi.nlm.nih.gov/protein)). In addition in contrast to DsA1 some strains (mostly MLST type II) exhibit a distinctly different PT repeat region composed strongly of polar and negatively charged (acidic) amino-acids. But also here few variants of this negatively charged stretch exist, which supports a functionally distinct role as a spacer (such as potentially compensating a lack of PT glycosyation in the affected strains), and not a generally hypervariable region. In a way the negative replacement combined with few variants of this sequence type particularly argues against hypervariability but rather for a distinct functional role requiring a specific physicochemical setup (negative charge, which also suspected bacterial glycosylations of PT repeats may normally provide).

For example, the N-terminus of Q6A5P9 contains certain amino acid exchanges compared to other DsA2 polypeptides of P. acnes contained in the sequence databases (see FIGS. 12A and 12C). Since these exchanges concern (besides the PT region) mainly the N-terminal signal sequence, these exchanges have no impact on the DsA2 polypeptide used as a vaccine according to the present invention (because these are usually provided in the mature form, i.e. without the signal sequence; the major exception is in case a nucleic acid vaccine is provided; this usually contains coding sequences of signal sequences (these signal sequences, however, may be adapted to the cells wherein these nucleic acid vaccines are designed to be expressed). Due to these differences in the N-terminus, there are several DsA2 sequences in the databases wherein the N-terminal alanine residue of the NSR (A72) is shifted to position 31 (A31 of EGR92169.1) or 21 (A21 in EFS48378.1), however, with identical sequences thereafter (until to the PT region).

The main differences in the DsA2 sequences in the CTR are either due to sequencing biases (due to the PT encoding region) which result in a (probably artificial) truncation (with a probable loss of function)) or concern specific exchanges, such as e.g. an R444H exchange.

A “fragment of a DsA1 or DsA2 polypeptide according to the present invention” is a shortened version of a naturally occurring version of a DsA1 or DsA2 polypeptide of P. acnes (see the definition of “fragments”, above). Preferably, the fragment according to the present invention comprises or consists at least of a CSD2 fragment, wherein the CSD2 fragment is (1) a contiguous polypeptide sequence of phenylalanine 150 (F150) to leucine 184 (L184), (2) a contiguous polypeptide sequence of phenylalanine 150 (F150) to leucine 267 (L267), or (3) a contiguous polypeptide sequence of histidine 218 (H218) to leucine 267 (L267) (and the corresponding fragments of DsA2, i.e. F194-L228, F194-L311 and H262-311, respectively). According to a preferred embodiment of the present invention, these three peptides may be defined as “minimum peptides” defining the minimum lengths of the polypeptides to be used in a vaccine according to the present invention, wherein protection as immunogen is safeguarded. Shorter peptides could be less reliable with respect to eliciting an appropriate immune response. On the other hand, the polypeptides should be as short as possible. Although shorter peptides are known to exhibit more convenient properties with respect to production and handling, protection provided by such shortened versions of native immunogens, such as DsA1 or DsA2, is both not predictable and unlikely.

Accordingly, it was surprising that vaccines comprising the N-terminal fragments provided with the present invention which lack the CTR containing the LPXTG motif are protective for treating and preventing P. acnes infections. Preferred fragments according to the present invention contain at least the CSD2 fragments disclosed above and (if present at all) a shortened PT repeat as defined above, i.e. none, one, two, three or five PT repeats. The fragments may also contain further domains, such as one or more of NSR, CSD1, SR1, CSD2, SR2 and CTR; either the full domains or parts thereof. The fragments according to the present invention may also consist of more than one fragment, e.g. a polypeptide containing the CSD2 fragments, CDS1, SR2, CSD3 and shortened PT repeat.

The “derivatives of a DsA1 polypeptide according to the present invention” comprise a fragment of a DsA1 or DsA2 polypeptide according to the present invention and further non-naturally occurring amino acid sequences (e.g. non-DsA1 or DsA2 sequences). The derivatives of a DsA1 or DsA2 polypeptide according to the present invention comprise at least the DsA1 or DsA2 fragment derived from a wild-type sequence of a P. acnes DsA1 or DsA2 protein and additionally contain at least one amino acid or amino acid sequence which—in combination with the fragment derived from the native sequence—define a sequence of the derivative which does not naturally occur. For example, if the fragment is derived from a native DsA1 protein, a “derivative” thereof may contain additional non-DsA1 sequences, e.g. sequences from a DsA2 protein. According to a preferred embodiment, the further sequences in these derivatives according to the present invention contain further immunogenic regions. Preferred derivatives according to the present invention may—besides CSD2 according to the present invention—contain further P. acnes sequences, especially further sequences encoding at least one (non-DsA1 or non-DsA2) antigen or epitope of P. acnes. For example, a derivative of the present invention, being derived (i.e. being a fragment of the native sequence by deletion) from DsA1, can contain at least one epitope of DsA2 of P. acnes. Preferably, the derivatives of the present invention comprise one or more of NSR, CSD1, SR1, CSD2, SR2, CSD3 and CTR of a DsA2 polypeptide of P. acnes, if the fragment is derived from a wild type DsA1 polypeptide; other preferred derivatives of the present invention comprise one or more of NSR, CSD1, SR1, CSD2, SR2, CSD3 and CTR of a DsA1 polypeptide of P. acnes, if the fragment is derived from a DsA2 polypeptide. These preferred derivatives therefore comprise sequences of DsA1 and DsA2. Derivatives which contain at least one DsA1 epitope and at least one DsA2 epitope provide that most balanced vaccination, because such a combined immunogen induces antibodies capable of equally good recognition of both DsA1 and DsA2; therefore inducing the most balanced reactivity against both DsA1 and DsA2, in contrast to full length proteins and other fragments, which reacted most highly against themselves but recognized the second antigen to a much lower extent, for example in strains with phylotype IC, where more DsA1 than DsA2 can be detected on the surface in contrast to others by flow cytometry. Therefore, the broadest and most balanced cross-reactivity profile on a large collection of strains covering different phylotypes can be achieved through the use of such a hybrid DsA1/DsA2 polypeptide (such as the Hybrid H4 molecule). Such hybrid polypeptides are therefore not only immunologically more consistent and simpler molecule that can exert the effect of both full-length proteins, but they have also a much more favorable stability and purity profile in comparison to their parent versions and are the preferred development candidates, especially if these hybrids include the functionally/immunologically most relevant regions confined mostly to the central and N-terminal part of these polypeptides (as in the H4 example which includes these parts of DsA1 (29-145; 278-333) and the central part of DsA2 (amino acids 190-321) and showed the ability to bind to both full length proteins in an ELISA assay (see example section, especially FIG. 8).

In a preferred embodiment of the present invention, the fragment or the derivative of the vaccine comprises or consists at least of
a contiguous polypeptide sequence of phenylalanine 150 (F150) to isoleucine 193 (I193),
a contiguous polypeptide sequence of phenylalanine 150 (F150) to leucine 267 (L267), or
a contiguous polypeptide sequence of histidine 218 (H218) to leucine 267 (L267).

Preferably, the fragment or the derivative according to the present invention does further not comprise a CSD1 of DsA1 or DsA2, an SR1 of DsA1 or DsA2, an SR2 of DsA1 or DsA2, a CSD3 of DsA1 or DsA2, or a PT repeat region of DsA1 or DsA2, preferably with the proviso that the fragment or derivative does further not comprise a CSD1 of DsA1 or DsA2, an SR1 of DsA1 or DsA2, an SR2 of DsA1 or DsA2, a CSD3 of DsA1 or DsA2, and a PT repeat region of DsA1 or DsA2.

A preferred fragment in the vaccine according to the present invention is (based on the numbering of DsA1, but also extending to the corresponding fragments of DsA2)

(1) a contiguous polypeptide sequence of phenylalanine 150 (F150) to leucine 184 (L184), further extending one, two, three, or four amino acids at the N-terminus and/or one, two, three, four, five, six, seven, eight, nine, or ten amino acids at the C-terminus;
(2) a contiguous polypeptide sequence of phenylalanine 150 (F150) to leucine 267 (L267), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus;
(3) a contiguous polypeptide sequence of histidine 218 (H218) to leucine 267 (L267) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus:
(4) a contiguous polypeptide sequence of phenylalanine 150 (F150) to leucine 267 (L267), further extending one, two, three, or four amino acids at the N-terminus and/or one, two, three, four, five, six, seven, eight, nine, or ten amino acids at the C-terminus;
(5) a contiguous polypeptide sequence of alanine 148 (F148) to leucine 271 (L271), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus;
(6) a contiguous polypeptide sequence of histidine 146 (H146) to threonine 277 (T277) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus;
(7) a contiguous polypeptide sequence of leucine 238 (L238) to leucine 272 (L272), further extending one, two, three, or four amino acids at the N-terminus and/or one, two, three, four, five, six, seven, eight, nine, or ten amino acids at the C-terminus;
(8) a contiguous polypeptide sequence of leucine 145 (L145) to alanine 201 (A201), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus; and/or
(9) a contiguous polypeptide sequence of leucine 145 (L145) to leucine 280 (L280) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus.

Preferably, the fragments according to the present invention have a length of at least 35 amino acids, preferably a length of at least 40 amino acids, especially a length of at least 50 amino acids.

Preferred vaccine according to the present invention comprise at least one additional polypeptide with an amino acid sequence containing or consisting of at least one additional P. acnes antigen or P. acnes epitope which is not a DsA1 or DsA2 antigen or epitope, preferably at least one additional amino acid sequence containing at least the PITP polypeptide of P. acnes or at least one epitope of PITP polypeptide of P. acnes, wherein the PITP polypeptide (see also: below) comprises from N- to C-terminus a signal peptide (“SP”; the SP is not part of the mature (functional) sequence and not part of the antigen, and not part in a production setup for the vaccine formulations disclosed herein; however, when produced in some form of continuous fermentation (i.e. when not harvesting and breaking open cells), secretion into the medium using a signal peptide may use appropriate SPs), an extended neocarzinostatin family domain (“ENFD”), a first swapping region (“SR1”), a heme binding domain (“HbD”), a second swapping region (“SR2”) which includes a C-terminal LPXT(G) domain, and a hydrophobic C-terminal region (“hLAR”). Preferably, this PITP derivative comprises at least one additional sequence from the PITP polypeptide, preferably a sequence comprising at least one of ENFD. SR1, SR2 and HbD, preferably selected from ENFD and HbD.

Since DsA1/DsA2 as well as PITP comprise spacer or swapping regions (“SRs”), the term “spacer region” or “swapping region” as used herein always refers to the SRs of the protein concerned; although the SRs have different sequences, they share the property of linking (“spacing” two structural domains of a given protein and are usually less ordered (intrinsically disordered) compared to the structural domains of the proteins, such as the CSDs, the ENFD or the HbD which may be combined by “swapping” the structural domains with appropriately engineering the “spacer/swapping regions” (i.e. keeping them at the same length (amino acid no.)). Usually, it is clear to which SR reference is made herein; however, in case of doubt, the term “SR” (unless specified) shall apply to all SRs, i.e. to the SR1 and SR2 of DsA1 and DsA1 as well as for SR1 and SR2 of PITP.

Another preferred derivative in the vaccines according to the present invention comprise at least one fragment of DsA1 and at least one fragment of DsA2.

Preferably, in the fragment or derivative of DsA1/DsA2 at least 5 PT repeats, preferably at least 10 PT repeats, especially at least 15 PT repeats, are deleted compared to a naturally occurring wild type DsA1/DsA2 polypeptide (“native DsA1/DsA2”), and wherein preferably at least one, more preferred at least two, more preferred at least three, even more preferred at least four, especially five, PT repeat(s) is/are present.

According to a preferred embodiment of the present invention, the derivative in the vaccine further comprises at least an NSR, CSD1, SR1, CSD2, SR2, CSD3 and/or CTR from at least one other DsA1 and/or DsA2 or from at least one other P. acnes strain than the strain from which the CSD2 fragment was derived.

Preferred DsA1/DsA2 fragments or derivatives lack the NSR, CTR, and/or the LPXTG motif. The DsA1/DsA2 according to the present invention, as well as its fragments and derivatives may be used in the present invention in the soluble form as well as in a membrane-bound form.

In a preferred embodiment, the vaccine according to the present invention further (in addition to CSD2 fragment or CSD2 containing fragment or DsA1 polypeptide) comprises another P. acnes antigen or epitope, preferably an antigen selected from DsA2 and PITP and/or a DsA2 epitope and/or an PITP epitope, especially an epitope containing fragment of another P. acnes polypeptide, preferably an epitope containing fragment of DsA2 and PITP.

Since computer predictions usually have biases, confirmation of epitopes (so that these epitopes become enabled epitopes with plausible function in an immunological environment, especially for use in vaccines) is dependent on “real world” wet biochemistry data with the actual immunogenic counterparts acting in an appropriate confirmation system. According to the present invention, epitopes have been analysed by using appropriate anti-DsA1, -DsA2, and anti-PITP-antibodies in linear, conformational, and MS epitope mapping, with additional antigen fragment mapping by ELISA and dot blot. This has provided the epitopes also disclosed in the example section.

Accordingly, further preferred fragments are fragments which contain or consist of immunogenic epitopes. Preferred epitopes are the ones identified by the present invention, namely R32-I41, Q38-K51, R32-K51, T43-K51, Q38-K51, R87-K90+T43-K51, R87-K90+T117-I132 and R87-K90+S234-G250, R87-K90+L246-A260, R87-K90+A256-E270, R87-K90+R266-T277, T117-I132, T117-A127, V128-I132, A144-N157, H146-A160, A148-N157, A156-A170, K166-L180, A176-T190, P186-A198, N181-E191, I216-F224, 1216-D225, A226-A240, S234-G250, I251-I263, I264-P271, P236-G250, L246-A260, A256-E270, S234-G250, I251-I263, I251-L267, A268-L280, R266-T277, T285-R286+I216-F224, T285-R286+I264-P271, T285-R286+V289-K296, T285-R286+V289-K296, T285-R286+A144-N157, A310-D313+T285-R286, T285-D290, T285-D290+V291-T300, T285-D290+A301-E307, V291-T300, A301-E307, T285-T300, A301-E307, R286-D290+V291-T300, R286-D290+A301-E307, R286-T300, V289-K296, A310-D313+I216-F224, A310-D313+I264-P271, A310-D313+T285-R286, A310-D313+R286-D290, A310-D313+V289-K296, A310-D313+V289-K296, A310-D313+T285-T300, A310-D313+A144-N157, A310-D313+T285-R286, A310-D313+T285-D290, A310-D313+T293-E307, T285-D290, V291-T300, T293-E307, A301-E307 of DsA1; L152-Q166, G190-P230, I199-D208, A218-I237, P230-Q244, I231-A270, H254-A270, A271-S279, A271-R310, L311-T321, L311-K323, V333-Q347, A218-P230, 1231-1237, H254-H262, Q256-H262, E261-D269, D269-S279, K313-K323, of DsA2: D79-10, E73-D85, R43-150, P68-Y75, P86-E92, 139-G45, Y84-D89, F81-D89, D79-T90, T37-E44, E73-W98, E73-F81, D89-10, P72-F81, A129-F138, D120-Q134, F111-D120, F132-G147, D152-E165, R115-F123, D120-K128, P131-F138, N181-E191, T143-T159, P116-T124, P131-D137, P131-D137, T175-C231, Q198-K203, P179-K185, G200-Q210, K174-A188, K174-K185, P201-Q209, P183-P201, P183-K191, K185-P195, R164-S180, E165-S180, K185-S190, V193-N202, V193-G200, K203-P208, R216-T225, R216-R224, P173-K191, K197-K203, P168-T175, K185-K203, R164-K174, T175-V193, S250-N261, D287-S300, K340-V347, D338-F352, D338-D348, S285-P288+G305-L314, S285-P288+H306-L314, S285-P288+T342-T351, S285-P288+D338-D348, D287-S300. T342-T351, D338-D348, H306-L314, G305-L314, G364-K375, R382-E399, V367-G373, A383-L390, T342-T351, M387-T395, E385-T392, V401-V410, N404-A409, G416-L427, L396-V410, T406-I415, D417-G424, V407-D418, V407-V414, K421-V429, S419-T430, D408-I415, T406-V414, of PITP.

According to a further aspect, the present invention therefore also provides a vaccine for use in the treatment or prevention of P. acnes-associated infections, comprising a polypeptide comprising an epitope of DsA1 and/or DsA2 and/or PITP, wherein the epitope is selected from the group consisting of R32-141, Q38-K51, R32-K51, T43-K51, Q38-K51, R87-K90+T43-K51, R87-K90+T117-I132 and R87-K90+S234-G250, R87-K90+L246-A260, R87-K90+A256-E270, R87-K90+R266-T277, T117-I132, T117-A127, V128-I132, A144-N157, H146-A160, A148-N157, A156-A170, K166-L180, A176-T190, P186-A198, N181-E191, I216-F224, I216-D225, A226-A240, S234-G250, I251-I263, I264-P271, P236-G250, L246-A260, A256-E270, S234-G250, 1251-1263, 1251-L267, A268-L280, R266-T277, T285-R286+I216-F224, T285-R286+I264-P271, T285-R286+V289-K296, T285-R286+V289-K296, T285-R286+A144-N157, A310-D313+T285-R286, T285-D290, T285-D290+V29I-T300, T285-D290+A30I-E307, V291-T300, A301-E307, T285-T300, A301-E307, R286-D290+V291-T300, R286-D290+A301-E307, R286-T300, V289-K296, A310-D313+I216-F224, A310-D313+I264-P271, A310-D313+T285-R286, A310-D313+R286-D290, A310-D313+V289-K296, A310-D313+V289-K296, A310-D313+T285-T300, A310-D313+A144-N157. A310-D313+T285-R286, A310-D313+T285-D290, A310-D313+T293-E307, T285-D290, V291-T300, T293-E307, A301-E307 of DsA1; L152-Q166, G190-P230, 1199-D208, A218-I237, P230-Q244, I231-A270, H254-A270, A271-S279, A271-R310, L311-T321, L311-K323. V333-Q347, A218-P230, I231-I237, H254-H262, Q256-H262, E261-D269, D269-S279, K313-K323, of DsA2; D79-T90, E73-D85, R43-150, P68-Y75, P86-E92, 139-G45, Y84-D89, F81-D89, D79-T90, T37-E44, E73-W98, E73-F81, D89-190, P72-F81, A129-F138, D120-Q134, F111-D120, F132-G147, D152-E165, R115-F123, D120-K128, P131-F138, N181-E191, T143-T159, P116-T124, P131-D137, P131-D137, T175-C231, Q198-K203, P179-K185, G200-Q210, K174-A188, K174-K185, P201-Q209, P183-P201, P183-K191, K185-P195, R164-S180, E165-S180, K185-S190, V193-N202, V193-G200, K203-P208, R216-T225, R216-R224, P173-K191, K197-K203, P168-T175, K185-K203, R164-K174, T175-V193, S250-N261, D287-S300, K340-V347, D338-F352, D338-D348, S285-P288+G305-L314, S285-P288+H306-L314, S285-P288+T342-T351, S285-P288+D338-D348, D287-S300, T342-T351, D338-D348, H306-L314, G305-L314, G364-K375, R382-E399, V367-G373, A383-L390, T342-T351, M387-T395, E385-T392, V401-V410, N404-A409, G416-L427, L396-V410, T406-1415, D417-G424, V407-D418, V407-V414, K421-V429, S419-T430, D408-1415, T406-V414, of PITP, preferably wherein the epitope comprises at least one additional amino acid residue at the N- or C-terminus of the DsA1, DsA2, or PITP sequence, especially wherein the epitope comprises at least two additional amino acid residue at the N- or C-terminus of the DsA1, DsA2, or PITP sequence; wherein the polypeptide is preferably covalently linked to a carrier molecule or embedded in a scaffold molecule, especially a carrier polypeptide. Again, the preferred maximum lengths of the epitope-carrying polypeptides are as disclosed herein; for example of less than 400 amino acids, preferably less than 350 amino acids, especially less than 300 amino acid residues (if, e.g. more than one or two epitopes of different proteins (DsA1, DsA2, PITP)) are shuffled in a single polypeptide; or a length of less than 250 amino acids, preferably less than 200 amino acids, especially less than 150 amino acid residues (if only one or more epitope of the same protein (DsA1, DsA2, PITP) is present on the polypeptide.

Another aspect of the present invention relates to DsA1 and/or DsA2 and/or PITP, and/or a fragment and/or a derivative of DsA1 and/or DsA2 and/or PITP according to the present invention for use in a therapeutic treatment, preferably for use in the treatment or prevention of P. acnes-associated infections, especially selected from the group consisting of acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes. Specifically, the DsA1 and/or DsA2 and/or PITP, and/or a fragment and/or a derivative of DsA1 and/or DsA2 and/or PITP according to the present invention is provided for use in the treatment or prevention of P. acnes-associated infections in a human patient suffering from P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes.

According to a preferred embodiment, the vaccine according to the present invention further comprises P. acnes PITP polypeptide and specific fragments and variants thereof.

A “PITP polypeptide of P. acnes” according to the present invention is a naturally occurring PITP protein of a P. acnes strain (“native PITP”) wherein the PITP polypeptide comprises from N- to C-terminus an extended neocarzinostatin family domain (“ENFD”), a first swapping region (“SR1”), a heme binding domain (“HbD”), a second swapping region (“SR2”) which includes a C-terminal LPXT(G) domain, and a hydrophobic C-terminal region (“hLAR”).

Accordingly, the functional domains of the PITP polypeptide (as referred to herein) are defined—on the basis of the numbering in and the sequence of Q6A9N1:

MTTSAMRKIV ASVLGAILAL TGVLITPAAW AAGPTVTVIP
VGREGGDITI SGKGFSTTGF GVYVAVAPAS VPEFYGNSDK
FYGYDPSKDT TESPSTIWVY TPSQKAIGSR FAQGRPMNND
GSFTITMKAP PFEQGKDFVV LTTKAHGVGK TDHSDDTRTP
VTYREATPAP TGPKTPIAPS KQPSKQAAPS KQVKPSKQAG
PNKQSTTPQQ KTAEHRSQTP AAHRTMTKQV CTIGASKVTS
GSLTWGIRTS FTSYLRGPIA NGSWKLSGGA NWNGSAFTFP
LTSGSFDPAT KSGSLKYSGS VHMTGHHGIL DMTLAEPSLQ
IKGSTGHLYL DVKSSSMDGK KTNYGRVDFA TFGVSVSGNA
AIKGSPVKLT ATGAKAFAGF YRAGEPMNPL STNLTLSAEK
VCHNVTVDAV TGKVIGDDSG KGAGRGLPVT GAEGPSSDEI
DLGIVGGLAL TAVVSTVVVC RRYAARI

—as follows (see also: e.g. FIG. 12D): ENFD from A32 to R164; SR1 from E165 to K237, HbD from V238 to L396, SR2 (including C-terminal LPXT(G) motif (i.e. including LPXT, but not the G)) from S397 to T430, and hLAR from G431 to 1467. The signal peptide (SP) of M1 to A31 is usually not contained in the final polypeptide (and in the vaccine); except the N-terminal methionine, which may be present.

Accordingly, an aspect of the present invention relates to a vaccine comprising the PITP polypeptide and/or a fragment and/or or a derivative of PITP, wherein the fragment and/or the derivative comprises or consists at least of a PITP epitope.

The vaccines of the present invention preferably contain a PITP polypeptide or a fragment or derivative thereof. A PITP “fragment” is a pan of a naturally occurring PITP protein; a PITP “derivative” is a non-natively occurring polypeptide which comprises a PITP fragment which contains at least an antigenic epitope (i.e. an epitope which is immunogenic and accessible to antibody binding on the surface of P. acnes) or has a length of at least 20 amino acids, preferably at least 30 amino acids, especially at least 50 amino acids of a naturally occurring PITP protein. The central domains of specifically preferred usefulness as vaccines are the ENFD and the HbD domain. Specifically preferred fragments and derivatives therefore comprise sequences from the ENFD and/or HbD domain, e.g. a fragment of at least of at least 10, preferably at least 20 amino acids, even more preferred at least 30 amino acids, especially at least 50 amino acids of a naturally PITP ENFD and/or HbD domain. Preferred ENFD fragments comprise the peptide from A32 to T143; preferred HbD fragments comprise the peptide from V238 to N393.

For the avoidance of doubt, it is also clear that also in connection with the PITP fragments and derivatives, all references to sequences, fragments, etc., herein always refer to consecutive amino acids (unless explicitly referred to the contrary). For example, a PITP fragment of at least 8 amino acids always refers to at least 8 consecutive amino acid residues of a PITP polypeptide. The term “consecutive” means that the given amino acid is at the given position in the alignment of FIG. 12. Interchanges between different PITP homologues are, of course, to be included, if the position is kept (i.e. no deletions, unless explicitly foreseen in FIG. 12).

Besides the SRs, also the adjacent portions of the ENFD and the HbD domain may be used as a spacer or linker between fragments e.g. in hybrid molecules. For example the region of K144 to T227 (or, including the whole SR1: of K144 to K237; or from G147 to K228; or any fragment thereof of at least 10 amino acids in length) can be used as an intermediate/swapping/spacer region (disordered linker joining e.g. ENFD and HbD). The hLAR is defined to start after the LPXT(G) according to the present invention, the terminal G of the LPXTG motif is already included in the hLAR, because this makes hLAR the hydrophobic part of the protein which is removed upon anchoring to the cell-wall. Accordingly, for practical reasons, the hLAR is defined for the sake of the present invention to start with the G of the LPXTG motif.

UniProt amino acid sequence Q6A9N1 refers to the protein “PPA0779” of P. acnes strain DSM 16379/KPA171202. In this wild type PITP polypeptide, the LPXT(G) motif is defined by amino acid numbers 427 to 431 (from L427 to G431; however. 0431 already forms part of hLAR) and the hLAR therefore starts with glycine at position 431 (0431) and ends with the C-terminal isoleucine at position 467 (I467). hLAR has (N-terminally) a rather hydrophilic region (including four acidic amino acids (E433, D438, E439 and D441) which ends with the aspartic acid residue at position 441 (D441) and then contains a hydrophobic region (starting from leucine at position 442 (L442) and extending to the C-terminal isoleucine). According to a preferred embodiment of the present invention, the hLAR may be completely deleted or partially deleted. Partial deletion concerns preferably the complete hydrophobic region (i.e. from L442 to 1467). This enables a preferred immunogenic fragment of this protein which has improved properties, especially also with respect to handling and manufacturing.

A preferred PITP fragment according to the present invention is a PITP with a shortened or completely deleted C-terminal region (hLAR), comprising all of the other domains (from N- to C-terminus): ENFD, SR1, HbD, SR2, including the LPXT(G) motif (i.e. comprising LPXT, but not the G). The hLAR located at the C-terminus is—in the preferred PITP polypeptide fragments according to the present invention—either shortened or not present at all (compared to native (i.e. naturally occurring) PITP proteins from P. acnes). The sequence of naturally occurring PITP polypeptides contained in the sequence database (see e.g. FIG. 12D) are highly identical and differ (besides point mutations) only in the N- and in the C-terminus. However, this difference and differences of some entries in the databases at the C-terminus may be either (sequencing) artefacts or are not native PITP polypeptides within the meaning of the present invention, i.e. that they do not function as PITP proteins in P. acnes. The sequence numbering used according to the present invention is based on the numbering of Q6A9N1 which means that e.g. the LPXTG motif preceding the hLAR is always referred to as extending from L427 to 0431 even if a PITP polypeptide (or fragment or derivative) with a different length (i.e. wherein e.g. L427 to 0431 are at a different amino acid number in this specific polypeptide) is concerned. The mRNA of PITP encodes also a N-terminal signal sequence (beginning with an N-terminal methionine residue and ending with a proline-alanine-alanine-tryptophane-alanine sequence) which is not part of the mature polypeptide. Accordingly, the PITP polypeptide of P. acnes” according to the present invention begins with the alanine residue 32 (A32) of the amino acid sequence Q6A9N1 in the UniProt database and ends with isoleucine 467 (I467).

For example, the N-terminus of Q6A9N1 contains certain amino acid exchanges compared to other PITP polypeptides of P. acnes contained in the sequence databases (see FIG. 12D). Since these exchanges mainly concern the N-terminal signal sequence, these exchanges have no impact on the PITP polypeptide used as a vaccine according to the present invention (because these are usually provided in the mature form, i.e. without the signal sequence: the major exception is in case a nucleic acid vaccine is provided; this usually contains coding sequences of signal sequences (these signal sequences, however, may be adapted to the cells wherein these nucleic acid vaccines are designed to be expressed). Differences in the hLAR may be due to sequencing biases.

Preferably, the PITP fragment and/or derivative is a PITP polypeptide wherein the hLAR is deleted, replaced by a hydrophilic C-terminal region, or partially deleted, wherein the partial deletion results in a loss of hLAR except the N-terminal 12 amino acids of hLAR, preferably except the N-terminal 11 amino acids of hLAR, especially except the N-terminal 10 amino acids of hLAR; or a fragment thereof or derivative thereof comprising at least amino acids corresponding to proline 34 to glutamic acid 73 or proline 94 to threonine 143 of ENFD or valine 238 to asparagine 393 of HbD in the amino acid sequence Q6A9N1 in the UniProt database. The fact these deletion fragments and derivatives of PITP show enhanced physicochemical properties which makes them particularly suitable for vaccination purposes and recombinant vaccine manufacturing was moreover unexpected also because the LPXTG motif is not automatically detected, meaning the motif it is not listed or annotated in the public databases for the present sequences. In the prior art, it has not been shown experimentally that PITP was actually anchored to the cell wall and could be an appropriate substrate for a transpeptidase). In the course of the present invention it was shown that LPXTG presence in the sequence does not affect the surface accessibility of PITP on different strains, because PITP could consistently be detected regardless of whether the LPXTG motif was present or not. Finally, also no corresponding sortase enzyme is apparent with relevance in P. acnes. Another surprising property of the PITP fragments or derivatives which have a deletion in the hLAR or wherein hLAR is deleted is an increased expression in many recombinant expression systems, especially in high-producing systems. In many of such systems, these expression products are present in the soluble fraction and not in inclusion bodies. As a further advantage, the PITP fragments or derivatives which have a deletion in the hLAR or wherein hLAR is deleted show an improved binding to typical purification columns used in polypeptide purification, such as SP Sepharose or Superdex 200 columns. Finally, also separation of the target polypeptide from (further) truncated forms or of other expression artefacts is improved. These effects can even be further pronounced by the replacement of cystein residues in the polypeptide, especially C231 and C402 replacements.

The PITP according to the present invention, as well as its fragments and derivatives may be used in the present invention in the soluble form as well as in a membrane-bound form.

Preferably, the DsA1/DsA2 derivative according to the present invention additionally comprises a PITP fragment and/or derivative is a PITP polypeptide as defined herein, especially wherein the hLAR is deleted, replaced by a hydrophilic C-terminal region, or partially deleted, wherein the partial deletion results in a loss of hLAR except the N-terminal 12 amino acids of hLAR, preferably except the N-terminal 11 amino acids of hLAR, especially except the N-terminal 10 amino acids of hLAR; or a fragment thereof or derivative thereof comprising at least amino acids corresponding to proline 34 to glutamic acid 73 or proline 94 to threonine 143 of ENFD or valine 238 to asparagine 393 of HbD in the amino acid sequence Q6A9N1 in the UniProt database.

Preferably, the PITP fragment or the derivative comprises or consists at least of
a contiguous polypeptide sequence of proline 34 to glutamic acid 73 of ENFD,
a contiguous polypeptide sequence of proline 94 to threonine 143, preferably of proline 94 to glycine 147, of ENFD, or
a contiguous polypeptide sequence of valine 238 to asparagine 393 of HbD.

According to a preferred embodiment, the PITP fragment or derivative contains a fragment of ENFD and/or of HbD of the PITP polypeptide with a length of at least 8 amino acid residues, preferably at least 10 amino acid residues, especially at least 15 amino acid residues. Alternative preferred embodiments comprise longer PITP fragments or derivatives with a length of at least 35 amino acids, preferably a length of at least 40 amino acids, especially a length of at least 50 amino acids.

Preferably, the PITP fragment or the derivative comprises or consists at least of an epitope of ENFD and/or an epitope of HbD, preferably wherein the fragment or the derivative comprises or consists at least of an epitope of ENFD and an epitope of HbD. Accordingly, a preferred embodiment of the PITP polypeptide, the PITP fragment or the PITP derivative comprises a porphyrin-binding domain.

Preferably, the PITP derivative further comprises at least an ENFD and/or HbD from at least one other PITP from at least one other P. acnes strain than the strain from which the PITP fragment or derivative has been derived.

According to a preferred embodiment, the fragment or derivative of the PITP polypeptide lacks at least one of the SRs, corresponding to serine 180 (S180) to glutamine 198 (Q198), preferably at least the amino acid sequence corresponding to proline 179 (P179) to threonine 207 (T207), especially the amino acid sequence corresponding to glutamic acid 165 (E165) to lysine 237 (K237), (for SR1), or valine 401 (V401) to threonine 430 (T430), preferably serine 397 (S397) to T430, especially leucine 394 to T430, (for SR2); in the amino acid sequence Q6A9N1 in the UniProt database.

Preferably, the fragment or derivative of the PITP polypeptide is a polypeptide, wherein at least the amino acid sequence corresponding to leucine 427 (L427) to glycine 431 (G431) is deleted, preferably wherein at least the amino acid sequence corresponding to proline 179 (P179) to glycine 431 (G431) is deleted, especially wherein at least the amino acid sequence corresponding to threonine 392 (T392) to glycine 431 (G431), is deleted; according to the numbering in the amino acid sequence Q6A9N1 in the UniProt database.

Preferred fragments or derivatives of the PITP polypeptide are polypeptides, wherein at least the amino acid sequence corresponding to serine 180 (S180) to glutamine 198 (Q198) and/or phenylalanine 74 (F74) to serine 93 (S93), preferably at least the amino acid sequence corresponding to proline 179 (P179) to threonine 207 (T207), especially the amino acid sequence corresponding to threonine 159 (T159) to threonine 219 (T219), in the amino acid sequence Q6A9N1 in the UniProt database is deleted.

Preferred fragments or derivatives of the PITP polypeptide according to the present invention consist of the following amino acids: A32 to T430, A32 to G426, A32 to Q198, A32 to T143, A32 to K400, A32 to T159, A32 to I177, A32 to Q204, A32 to G234, A32 to R164, A32 to S391, A32 to P179, A32 to R158, A32 to G147, A32 to E73 and P94 to G147; P34 to T430, P34 to G426, P34 to Q198, P34 to T143, P34 to K400, P34 to T159, P34 to I177, P34 to Q204, P34 to G234, P34 to R164, P34 to S391, P34 to P179, P34 to R158, P34 to G147, P34 to E73 and P94 to G147; S240 to S391, A32 to D441, A32 to I440, A32 to E439, A32 to D438, A32 to S437, A32 to S436, A32 to P435, A32 to G434, A32 to E433, A32 to A432, A32 to G431; P34 to D441, P34 to I440, P34 to E439, P34 to D438, P34 to S437, P34 to S436, P34 to P435, P34 to G434, P34 to E433, P34 to A432, P34 to G431; S240 to D441, S240 to I440, S240 to E439. S240 to D438, S240 to S437. S240 to S436, S240 to P435, S240 to G434, S240 to E433, S240 to A432, S240 to G431; A32 to T430, A32 to V429, A32 to P428, A32 to L427, A32 to G426, A32 to R425, A32 to G424, A32 to A423, A32 to G422, A32 to K421, A32 to G420, A32 to S419. A32 to D418, A32 to D417, A32 to G416, A32 to I415, A32 to V414, A32 to K413, A32 to G412, A32 to T411, A32 to V410, A32 to A409, A32 to D408, A32 to V407, A32 to T406, A32 to V405, A32 to N404, A32 to H403, A32 to C402, A32 to V401, A32 to K400, A32 to E399, A32 to A398, A32 to S397, A32 to L396, A32 to T395, A32 to L394, A32 to N393, A32 to T392; P34 to T430, P34 to V429, P34 to P428, P34 to L427, P34 to 0426, P34 to R425, P34 to G424, P34 to A423, P34 to G422, P34 to K421, P34 to G420, P34 to S419, P34 to D418, P34 to D417, P34 to G416, P34 to I415, P34 to V414, P34 to K413, P34 to G412, P34 to T411, P34 to V410, P34 to A409, P34 to D408, P34 to V407, P34 to T406, P34 to V405, P34 to N404, P34 to H403, P34 to C402, P34 to V401, P34 to K400, P34 to E399, P34 to A398, P34 to S397, P34 to L396, P34 to T395, P34 to L394, P34 to N393, P34 to T392; G172 to T430, G172 to V401, G172 to K400, G172 to L396, G172 to N393, A199 to T430, A199 to V401, A199 to K400, A199 to L396, A199 to N393, H223 to T430, H223 to V401, H223 to K400, H223 to L396, H223 to N393, T232 to T430, T232 to V401, T232 to K400, T232 to L396, T232 to N393, G234 to T430, G234 to V401, G234 to K400, G234 to L396, G234 to N393, V238 to T430, V238 to V401, V238 to K400, V238 to L396, V238 to N393, S240 to T430, S240 to V429, S240 to P428, S240 to L427, S240 to G426, S240 to R425, S240 to G424, S240 to A423, S240 to 0422, S240 to K421, S240 to G420, S240 to S419, S240 to D418, S240 to D417, S240 to G416, S240 to 1415, S240 to V414. S240 to K413, S240 to G412, S240 to T411, S240 to V410, S240 to A409, S240 to D408, S240 to V407, S240 to T406, S240 to V405, S240 to N404, S240 to H403, S240 to C402, S240 to V401, S240 to K400, S240 to E399, S240 to A398, S240 to S397, S240 to L396, S240 to T395, S240 to L394, S240 to N393, S240 to T392.

According to a preferred embodiment, the present invention relates to PITP fragments of at least 8 amino acids and derivatives containing such fragments. Preferably, the fragments or derivatives containing such fragments are at least 9 amino acids in length, especially at least 10 amino acids. The following list includes preferred 10mer fragments (in the following, an alternative amino acid annotation is used; however, it is clear that e.g. the term “391” is “139” and “isoleucine 39” as otherwise used herein):

39I-481, 40P-49T, 41V-50I, 42G-51S, 43R-52G, 44E-53K, 68P-77N, 69A-78S, 70S-79D, 71V-80K, 72P-81F, 73E-82Y, 74F-83G, 75Y-84Y, 76G-85D, 77N-86P, 78S-87S, 79D-88K, 80K-89D, 81F-90T, 82Y-91T, 830-92E, 84Y-93S, 85D-94P, 86P-95S, 87S-96T, 88K-97I, 89D-98W, 90T-99V, 91T-100Y, 92E-101T, 93S-102P, 94P-103S, 95S-104Q, 96T-105K, 971-106A, 98W-1071, 99V-108G, 100Y-109S, 101T-110R, 102P-111F, 103S-112A, 104Q-113Q, 105K-114Q, 106A-115R, 107I-116P, 108G-117M, 109S-118N, 110R-119N, 111F-120D, 112A-121G, 125I-134Q, 126T-135G, 127M-136K, 128K-137D, 144K-153H, 145A-154S, 146H-155D, 147G-156D, 148V-157T, 149G-158R, 150K-159T, 151T-160P, 152D-161V, 153H-162T, 154S-163Y, 155D-164R, 156D-165E, 157T-166A, 158R-167T, 159T-168P, 160P-169A, 161V-170P, 162T-171T, 163Y-172G, 164R-173P, 165E-174K, 166A-175T, 167T-176P, 168P-177I, 169A-178A, 170P-179P, 171T-180S, 172G-181K, 173P-182Q, 174K-183P, 175T-184S, 176P-185K, 177I-186Q, 178A-187A, 179P-188A, 180S-189P, 181K-190S, 182Q-191K, 183P-192Q, 184S-193V, 185K-194K, 186Q-195P, 187A-196S, 188A-197K, 189P-198Q, 190S-199A, 191K-200G, 192Q-201P, 193V-202N, 194K-203K, 195P-204Q, 196S-205S, 197K-206T, 198Q-207T, 199A-208P, 200G-209Q, 201P-210Q, 202N-211K, 203K-212T, 204Q-213A, 205S-214E, 206T-215H, 207T-216R, 208P-217S, 209Q-218Q, 210Q-219T, 211K-220P, 212T-221A, 213A-222A, 214E-223H, 215H-224R, 216R-225T, 217S-226M, 218Q-227T, 219T-228K, 220P-229Q, 221A-230V, 222A-231C, 223H-232T, 224R-233I, 225T-234G, 226M-235A, 227T-236S, 228K-237K, 229Q-238V, 230V-239T, 231C-240S, 232T-241G, 233I-242S, 266L-275S, 267S-276A, 268G-277F, 282T-291K, 334S-343N, 353G-362I, 354V-363K, 355S-364G, 356V-365S, 357S-366P, 358G-367V, 359N-368K, 377F-386P, 378A-387M, 379G-388N, 380F-389P, 396L-405V, 397S-406T, 398A-407V, 399E-408D, 400K-409A, 401V-410V, 402C-411T, 403H-412G, 404N-413K, 405V-414V, 406T-4151, 407V-416G, 408D-417D, 409A-418D, 410V-419S, 411T-420G, 412G-421K, 413K-422G, 414V-423A, 4151-424G, 416G-425R, 417D-426G, 418D-427L, 419S-428P, 420G-429V, 421K-430T, 422G-431G, 423A-432A, 424G-433E, 425R-434G, 426G-435P, 427L-436S, 428P-437S, 429V-438D, 430T-439E, 431G-440I, 432A-441D, 433E-442L, 434G-443G, 435P-444I, 436S-445V, and 437S-446G.

Preferred fragments and derivatives of PITP comprise at least the following PITP fragments: I39-K53, P68-G121, I125-D137, K144-S242, L266-F277, T282-K291, S334-N343. G353-K368, F377-P389, and L396-G446.

Specifically preferred fragments and derivatives of PITP comprise at least or consist of the following PITP fragments: A32 to R164, A32 to Q198, A32 to T143, A32 to V148, A32 to T171, P34 to R164, A32 to T159, A32 to I177, A32 to Q204, A32 to G234, A32 to K400, A32 to S391, V238 to K400, A199 to T430, V238 to T395, G234 to K400, H223 to K400, T232 to V401, V238 to T392, V238 to N393, V238 to L394, V238 to T395, V238 to L396, T232 to T430, G172 to K400, and G172 to G234, especially the fragments A32-T430, A32-I467, A32-S391 (with K174-T239 deleted), A32-S391. These fragments contain at least one efficient epitope to elicit an appropriate, cross-type reactive immune reaction if provided as a vaccine in humans. Moreover, these fragments are efficiently producible by recombinant expression systems and finishable in final pharmaceutical vaccine formulations.

According to a specifically preferred embodiment, the fragments and derivatives of PITP comprise a fragment which includes the complete or almost complete HbD domain, i.e. at least amino acids V238 to T392, or at least amino acids V238 to N393, or at least amino acids V238 to L394, or, even more preferred at least amino acids V238 to T395 or at least amino acids V238 to L396. Such fragments are stable and suitable for expression also in up-scaled formats. HbD fragments wherein T392 to T395, N393 to T395, L394 to T395 or T395 is missing at the C-terminus of this domain may be less stable with respect to expression and epitope presentation.

According to a specifically preferred embodiment, the fragments and derivatives of PITP comprise a fragment which includes the complete HbD domain, i.e. at least amino acids V238 to T392, up to L396. Such fragments are stable and suitable for expression also in up-scaled formats. HbD fragments wherein T392, T392 to N393, T392-L394 or T392-L395 is missing may be less preferred for certain purposes, such as large scale production.

According to a preferred embodiment of the present invention, modified PITP polypeptides are provided which have advantageous properties compared to wild type PITP proteins from P. acnes and are specifically suitable for vaccination purposes. The novel use of PITP proteins according to the present invention for being used to interfere with (i.e. prevent and/or treat pathological conditions caused by) P. acnes is based on their advantageous properties (as revealed by the present invention), both with respect to their immunogenic properties as well as with respect to their handling properties (which enable easier large-scale recombinant expression and production). Both advantages appeared in the course of generation of the present invention and are surprising in view of the knowledge in the art.

DsA1 and DsA2 can be considered fairly invariant when sequencing issues and pseudo-genes are disregarded and PT-length polymorphism is viewed from a functional role of the region rather than a specific amino-acid to amino-acid comparison. Yet PITP (a putative iron transporter) is even more conserved. Length variants are rare and in most cases most likely due to uncertainties to place the N-terminal gene start correctly. Particularly when disregarding a few apparently fragmented or shifted proteins the sequence is highly invariant. In essence over the entire protein among known variants only a few dozen positions show variability at all. One exception is GAE78839.1, a possible fusion with a downstream gene. One of the few regions showing an enrichment also in chemically dissimilar amino-acid exchanges is the putative linker region joining the two predicted heme binding domains, particularly in the area defined by peptide TTPQQKTAEH.

According to a specifically preferred embodiment, the present invention relates to shortened fragments and variants of the P. acnes PITP polypeptide, wherein the hLAR is deleted, replaced by a hydrophilic C-terminal region, or partially deleted, wherein the partial deletion results in a loss of hLAR except the N-terminal 12 amino acids of hLAR, preferably except the N-terminal 11 amino acids of hLAR, especially except the N-terminal 10 amino acids of hLAR; or a fragment thereof or derivative thereof comprising at least ENFD or HbD.

Preferred derivatives of the present invention are polypeptides which comprise sequence stretches of different antigens of P. acnes. Accordingly, the present invention refers to a specific aspect to a polypeptide comprising at least one polypeptide stretch of Dermatan sulfate-binding adhesin 1 of P. acnes (DsA1) and at least one polypeptide stretch of Dermatan sulfate-binding adhesin 2 of P. acnes (DsA2), said DsA1 and DsA2 comprising from N- to C-terminus an N-terminal region, a first conserved sub-domain (“CSD1”), a first swapping region (“SR1”), a second conserved sub-domain (“CSD2”), a second swapping region (“SR2”), a third conserved sub-domain (“CSD3”); and, optionally a Pro-Leu repeat containing region (“PT repeat region”), and a C-terminal region;

wherein the polypeptide comprises at least CSD1, CSD2 or CSD3 of DsA1 and at least CSD1, CSD2 or CSD3 of DsA2.

According to another aspect, the present invention refers to a polypeptide comprising at least one polypeptide stretch of Dermatan sulfate-binding adhesin 1 of P. acnes (DsA1) and at least one polypeptide stretch of Dermatan sulfate-binding adhesin 2 of P. acnes (DsA2), said DsA1 and DsA2 comprising from N- to C-terminus an N-terminal region, a first conserved sub-domain (“CSD1”), a first swapping region (“SR1”), a second conserved sub-domain (“CSD2”), a second swapping region (“SR2”), a third conserved sub-domain (“CSD3”): and, optionally, a Pro-Leu repeat containing region (“PT repeat region”), and a C-terminal region; wherein the polypeptide stretch of DsA1 and DsA2 has independently a length of at least 20 amino acid residues.

Another preferred aspect of the present invention is a vaccine comprising
at least one antigenic polypeptide with an epitope of a DsA1 polypeptide which is surface exposed or an epitope of a dermatan sulfate-binding adhesin 2 of P. acnes (DsA2 polypeptide) which is surface exposed, wherein DsA1 or DsA2 comprises from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), and, optionally, a Pro-Thr repeat containing region (“PT repeat region”), and a C-terminal region (“CTR”); and
at least one antigenic polypeptide with an epitope of a PITP polypeptide which is surface exposed.

Another preferred aspect of the present invention is a vaccine comprising a polypeptide

(a) comprising at least an epitope-containing fragment or derivative of CSD1 of DsA1 and at least an epitope-containing fragment or derivative of CSD2 of DsA2; and/or
(b) comprising at least a polypeptide stretch comprising a CSD1 fragment or derivative of CSD1 of DsA1 of at least 30 amino acid residues, preferably at least 40 amino acid residues, and at least a polypeptide stretch comprising a CSD2 fragment or derivative of CSD2 of DsA1 of at least 30 amino acid residues, preferably at least 40 amino acid residues;
(c) comprising at least
a contiguous polypeptide sequence of phenylalanine 150 (F150) to leucine 184 (L184),
a contiguous polypeptide sequence of phenylalanine 150 (F150) to leucine 267 (L267), or
a contiguous polypeptide sequence of histidine 218 (H218) to leucine 267 (L267) of DsA1, and
a contiguous polypeptide sequence of phenylalanine 194 (F194) to leucine 228 (L228),
a contiguous polypeptide sequence of phenylalanine 194 (F194) to leucine 311 (L311), or
a contiguous polypeptide sequence of histidine 262 (H262) to leucine 311 (L311) of DsA2.

Also in this aspect, it is preferred that the polypeptide comprises an amino acid exchange at one or more of C53, C319 and C321 of DsA1, and C97 and C363 of DsA2, if present in the polypeptide, preferably one or more of amino acid exchanges C53S, C319S and C321P of DsA1 and C97S and C363S of DsA2.

Also in this aspect, it is preferred that the fragment or derivative at least 5 PT repeats, preferably at least 10 PT repeats, especially at least 15 PT repeats, are deleted compared to a naturally occurring wild type DsA1/DsA2 polypeptide (“native DsA1/DsA2”), and wherein preferably at least one, more preferred at least two, more preferred at least three, even more preferred at least four, especially five, PT repeat(s) is/are present.

Also in this DsA1/DsA2 shuffle polypeptide, it is preferred to include a PITP stretch, especially a PITP stretch with a PITP epitope. Accordingly, this polypeptide preferably further comprises a PITP polypeptide or a fragment or derivative of PITP comprising a polypeptide stretch of at least 30 amino acid residues comprising at least an epitope of PITP, preferably wherein the PITP polypeptide or a fragment or derivative of PITP comprises at least
a contiguous polypeptide sequence of proline 34 to glutamic acid 73 of ENFD or a PITP derivative thereof,
a contiguous polypeptide sequence of proline 94 to threonine 143 of ENFD or a PITP derivative thereof, or
a contiguous polypeptide sequence of valine 238 to asparagine 393 of HbD or a PITP derivative thereof.

Again, also here, the PITP derivative preferably comprises an amino acid exchange exchange at positions C231 and C402.

In a similar aspect, the present invention also relates to a vaccine comprising
at least one synthetic antigenic polypeptide with an epitope of a DsA1 polypeptide which is surface exposed or an epitope of a DsA2 polypeptide which is surface exposed; and
at least one synthetic antigenic polypeptide with an epitope of a PITP polypeptide of P. acnes which is surface exposed.

The antigens as described herein, or epitopes thereof may not be inherently immunogenic and therefore are linked to each other and/or to an adjuvant to produce an immunogen. The linkage between the antigen(s) and/or epitope(s) thereof and/or the adjuvant may be covalent or non-covalent, for example by, adsorption, electrostatic, hydrophobic or through van der Waals interactions.

In some embodiments, the immunogen is a composite immunogen and is engineered by linking of one or more antigen(s) of P. acnes and/or one or more epitope(s) of P. acnes to each other. In some embodiments, the immunogen comprises or consists of at least 2 (e.g. 2, 3, 4, 5, 6, 7, 8, 9, or 10) antigen(s) and/or epitope(s) thereof. The at least two antigens and/or epitopes thereof may be the same or different antigens and/or epitopes (e.g. two epitopes of two different antigens, two different epitopes of one antigen, two copies of the same epitope of one antigen, one antigen and one epitope of a different antigen). In some embodiments, the immunogen comprises or consists of at least two (e.g., 2, 3, 4, 5, 6 7, 8, 9, or 10) antigen(s) of P. acnes and/or epitope(s) thereof linked to an adjuvant, wherein at least one of the epitopes induces cross-reactive antibodies, especially cross-type-reactive antibodies.

In some embodiments, the immunogen comprises at least two antigen(s) and/or epitopes thereof covalently linked to each other. In some embodiments, the immunogen comprising at least two antigen(s) and/or epitopes thereof are further linked to an adjuvant.

In some embodiments, the immunogen comprises one antigen or epitope thereof linked to an adjuvant.

The immunogen is particularly characterized by the presence of at least one epitope that induces cross-binding and/or cross-reactive antibodies, especially cross-type-reactive antibodies.

The term “cross-binding” as used herein shall refer to antibodies raised by immunization using the vaccine which specifically bind more than one antigen. Cross-binding antibodies are antibodies that when raised by a single antigen (e.g. by a habrid molecule, such as H4), can specifically bind two or more antigens (e.g. DsA1 and DsA2).

Cross-binding of antibodies induced after immunization with an antigen or epitope can be determined as follows. Sera from rabbits or mice immunized multiple times with specific P. acnes antigens alone or in combination with an adjuvant can be evaluated for the amount of antibody binding to the antigens used for immunization compared to other non-related P. acnes proteins. Considering equal quality and the purity of the recombinant protein antigens (immunogens), immunization with a particular P. acnes antigen leads to a substantial increase in the amount of antigen-specific antibodies, with the EC50 titers determined by ELISA. The detection of ELISA EC50 titers of the animal sera raised against the antigens and against non-related P. acnes antigens not used for immunization of the animals, is a clear indication of cross-binding of the antigen-specific polyclonal serum. In such an assay, serial dilutions of hyperimmune rabbit sera are tested in an ELISA and the EC50 titer of an antibody in the serum or other bodily liquids is the concentration (dilution) in which half maximal antigen binding effect is observed (generally read out as optic density, O.D.). Pre-immune sera derived from the respective rabbit prior to immunization are used to control for unspecific serum effects.

The term “cross-reactive” (or “cross-reactivity”) as used herein shall refer to antibodies raised by immunization using the vaccine which specifically bind more than one P. acnes strain. The term “cross-type-reactive” (or “cross-type-reactivity”) as used herein shall refer to antibodies raised by immunization using the vaccine which specifically bind more than one P. acnes phylotype. In particular, cross-reactivity, in particular cross-type-reactivity, cover the specific binding of live bacterial cells from at least two of the phylotypes or ribotypes of P. acnes strains, in particular at least two of Type IA1, IA2, IB, IC, II and III P. acnes, e.g. as determined by the reactivity regarding at least one strain of each type of P. acnes which expresses at least one of the antigens. For example, the cross-reactivity covers the specific reactivity of antibodies targeting only one antigen of P. acnes, however, cross-reacting with the same antigen or analogous antigens expressed by different strains. For example, a vaccine containing DsA1, DsA2 or fragments or derivatives as disclosed herein, elicits a cross-type reactivity at least against Types IA1, IA2, IC and II; a vaccine containing PITP or fragments or derivatives as disclosed herein, elicits a cross-type reactivity at least against Types IA1, IA2, IB, IC, II and III (see e.g. FIG. 1B, 1C).

Cross-binding and cross-reactivity/cross-type-reactivity of vaccine-induced antibodies may be tested with a surface-binding assay. The term “surface binding assay” as used herein refers to the following test procedure: Sera from rabbits or mice immunized multiple times with specific P. acnes proteins in combination with an adjuvant, or without needing an adjuvant, can be evaluated for their ability to bind the surface of P. acnes strains from different genetic types. An antigen surface expression and its accessibility to the humoral immune system can be verified by the ability of the antigen-specific antibodies to specifically bind the native antigen on the surface of live bacteria. The amount of antibody that specifically binds to the bacterial surface is measured by a flow cytometer which quantitates the amount of light emitted by the fluorescently labeled species-specific antibodies, recognizing the antibodies raised in this species and binding to specific surface epitopes. Binding thereby is expressed as median fluorescent intensity (MFI). Antigen-specific rabbit and mouse sera showing a substantial binding of antibodies to multiple P. acnes isolates (positive threshold of MFI at least 3-fold, preferably at least 5-fold increase over the MFI of the negative control such as corresponding preimmune serum, a pool of pre-immune sera or the sera generated by the immunization with the same formulation not containing the respective antigen, e.g. adjuvant or a physiological buffer control) is a clear indication for P. acnes strain/type cross-reactivity.

The cross-reactivity and cross-type-reactivity of vaccine-induced antibodies may be also tested in functional assays. The term “functional assay” as used herein shall refer to assays, which employ structures of a pathogenic cell specifically bound by the antibody, and a read-out to determine the effect of antibody binding to said pathogenic cell structures. Such read-out as used herein may be the cell killing (such as to determine the antibacterial activity), growth inhibition and/or neutralization of invasion of the mammalian cells. The respective functional assay may, thus, be an assay for determining pathogen cell killing, inhibition and/or neutralization of cellular growth, invasion of host cells, reduction in biofilm formation capacity or some other disease-associated function. Thereby, the antibacterial activity of an antibody-containing serum fraction may be tested as described herein. For example, sera from animals immunized multiple times with specific P. acnes proteins with or without an adjuvant, or human sera containing P. acnes-specific antibodies, can be evaluated for their ability to opsonize and induce killing of P. acnes strains from different genetic types by phagocytic cells such as granulocytes, neutrophils, macrophages, monocytes, dendritic cells, mast cells and any other cell capable of uptake and killing of the P. acnes bacterium in the presence of serum or other tissue liquids containing P. acnes-specific antibodies. Preferably, a mixture comprising neutrophils and granulocytes is used in these assays.

The term “antibacterial activity” as used herein shall mean any effect of a compound on a bacterium, directly or indirectly, e.g. upon eliciting an immune response, which effect is blocking or inhibiting the bacterium or the pathogenesis caused by the bacterium, including e.g. bactericidal, bacteriostatic, neutralizing or any other functional effect which reduces the virulent potential of the bacterium, such as interfering with growth or adhesion to human cells, reducing biofilm formation or reducing secretion of bacterial proteins.

The anti-bacterial activity is evidenced by a reduction of bacterial cell counts (colony forming units—CFU) recovered at the end of an opsonophagocytosis killing assay (OPK; preferably as performed in the example section of the present invention, below), e.g. at least 50% more reduction compared to negative control (immune serum from an animal immunized with a buffer not containing the antigen, and/or corresponding pre-immune serum taken before the first immunization with the respective antigen, and/or a sample containing all assay reaction components except serum): in at least two-fold higher dilution as used in the opsonophagocytic killing assay for K50 titer determination (or at least 60%, or at least 70%, or at least 80%, or at least 90%), of bacterial cell counts in a reaction sample, after an incubation of at least 24 h or longer using at least two serial two-fold dilutions of the antibody or serum starting at dilution of at least 1/200-1/1000 (e.g. from 1/200 dilution reaching at least 50% of the CFU reduction at 1/800), preferably at least four and up to 7 serial dilutions (1/200-1/25,600), even more preferably at least eight or nine serial dilutions (starting from 1/200 to 1/128,000) and the most preferably 10 or more serial dilutions (e.g. 1/200-1/204,800). It is understood that the antibacterial activity should be demonstrated in at least two dilution steps, more preferably in five or more dilution steps so that the percentage of bacterial killing declines from >90% down to >50%, or remains above 50% in at least two dilution steps and the last of the serial dilution in which the % of OPK activity is still above 50% before declining is defined as K50 titer of the tested antibody or a serum sample (see the preferred performance of an OPK assay, as described in detail in the example section, below, according to which—in cases of doubt—the OPK activity according to the present invention is determined). As a positive control, sera obtained from vaccinated individuals or individuals who have developed antibodies against P. acnes may be used or a polyclonal, e.g. animal or human, serum generated against a whole bacterium or a bacterial lysate which contains antibodies capable of killing, inhibiting or neutralizing bacterium. Negative control may be a corresponding pre-immune serum or a non-immune serum (e.g. a serum raised by the immunization using a physiological buffer or adjuvant without the addition of antigen).

An exemplary functional assay to determine antibacterial activity is serum bactericidal assay (SBA) (Taylor. 1983) or opsonophagocytosis killing assay (OPKA or OPK assay) (Gordon, 2016). For example, serum antibodies or sera can be tested in an opsonophagocytic killing assay, with the ability to opsonize bacteria for uptake and killing by phagocytes, indicating protective efficacy.

Vaccine induced antibodies can function in the process known as opsonization. Opsonization is a process by which microbial pathogens are targeted for ingestion by phagocytic cells of the immune system. The binding of opsonins attracts phagocytic cells which results in destruction of the bacterial pathogen. Phagocytosis is mediated by macrophages, granulocytes or other cells which are able to kill the bacterium and involves the ingestion and digestion of microorganisms, damaged or dead cells, cell debris, insoluble particles and activated clotting factors. Opsonins are agents which facilitate the phagocytosis of the above foreign bodies. Opsonic antibodies are therefore antibodies which provide the same function.

An exemplary functional assay to determine bacteriostatic function is growth inhibition assay, bacteriostasis assay or disk diffusion susceptibility method for measuring bacteriostatic range (inhibition zone) of a tested agent.

An exemplary functional assay to determine bacterial neutralization function is neutralization of cell/tissue adhesion, neutralization of bacterial binding to host molecules (e.g. neutralization of binding complement, fibrinogen or other plasma proteins), neutralization of toxins, enzymes and enzymatic activities that regulate growth, tissue invasion and spreading, or neutralization of cell-to-cell interactions and formation of biofilms.

In certain embodiments, the functional assay provides for the characterization of an antibody by its effect on the pathogen which differs from functionality as determined by simple binding assays, because the binding assays only determine (specific) binding properties of antibodies. Thus, a functional assay may differentiate between protective antibodies and simple specific binders.

Any immunologically relevant target antigen or epitope as described herein may be characterized by its function to elicit antibodies with antibacterial activity, as determined in a functional assay. Such functional assay e.g. employs the target antigen as isolated molecule or structure in a specific amount, the respective pathogen with the cell surface expressing the target antigen, and an antibody directed to said target antigen. In the functional assay, the effect of said antibody on the pathogen is determined with and without the presence of the isolated target antigen. If the target antigen or epitope is immunologically relevant, the anti-bacterial function of the antibody against this target antigen or epitope will be significantly inhibited by the presence of the competitive amount of isolated target antigen or epitope or by pre-incubation of the antibody with the target antigen or epitope.

An exemplary functional assay to determine the inhibition of a protective antibody are the bactericidal inhibition assay and antibody depletion assay. In these assays, a target antigen is used to inhibit or deplete the bactericidal antibodies from the tested serum or a purified antibody sample, thereby demonstrating that the selected antigen was indeed the target of bactericidal antibodies and therefore a good vaccine candidate. Antigens derived from the pathogens which are able to also infect animals and for which a good animal model exist, can be similarly evaluated in vivo (e.g. demonstrating that the antigen is able to inhibit or reduce protection due to serum transfer, if the serum is pre-incubated with the same antigen to deplete antigen-specific antibodies).

In general, the bactericidal activity of hyperimmune P. acnes sera or an antibody induced after vaccination with a protective vaccine is determined as a K50 titer referring to the highest dilution showing more than 50% decrease in bacterial counts in comparison to a negative control of the same dilution. Adsorption of the antigen-specific antibodies in the serum against the selected protein leads to removal of antigen specific antibodies resulting in a decrease in bactericidal activity of the serum in comparison to a non-adsorbed sample of the same dilution. This reduction in bactericidal activity can be also used as a direct correlate for the extent of antibacterial activity of antibodies in the respective human serum against the evaluated target antigen.

Other functional assays that can be used for testing the protective function of antibodies induced after immunization with vaccines comprising immunologically relevant antigens or epitopes are serum assays that measure the ability of antibodies to inhibit bacterial growth, adhesion, biofilm formation, inhibition of nutrient acquisition, secretion of toxins or immunomodulatory signaling molecules (e.g., those that inhibit complement activation or cytokine functions).

The term “variants” as used herein with respect to a protein which is an antigen or which comprises one or more epitopes as described herein, shall refer to anything other than the comparable or parent protein, which has substantially the same functional activity. Specifically preferred variants are referred to herein as “derivatives”, especially in the embodiment and in the claim section. The variant may e.g. be the same type of protein as the comparable one, yet, be derived or originating from a different bacterial strain, or an analog protein. The variant may e.g. be a derivative of a native protein that serves as a parent protein for generating variants and derivatives, respectively. In particular, the variant is derived from or relates to a P. acnes protein identified by the UniProt accession numbers Q6A5X9, Q6A5P9, Q6A9N1, or an analogous protein or fragment of any of the foregoing, or may be an analog protein or a non-naturally occurring artificial proteinaceous substance or protein, e.g. comprising or consisting of at least about 90% amino acid sequence identity, preferably at least 95%, more preferably at least 98%, more preferably at least 99% or at least 99.5%, to the amino acid sequence of such protein identified by the UniProt accession numbers Q6A5X9, Q6A5P9, Q6A9N1 or at least any of 65%, 70%. 75%, 80%, 85%, 90%, or 95% sequence identity.

Sequence identity is preferably (and in the cases of any unclarity or doubts) determined by using the clustal omega multiple sequence alignment software algorithm, version 1.2.4, obtained from EMBL-EBI. Clustal omega is suitable software for determining the global sequence alignment. It will not produce a sequence identity figure, i.e. no number, but an alignment. This alignment part is the crucial step, however, because determining identity can be done manually or with a simple script comparing identity of letters. Whatever software or algorithm is used instead, it should do global rather than local alignments if the entire protein is to be compared. A local algorithm such as BLAST would leave out non-matching regions in the flanks (in the N- and C-termini). Specifically the artificial N-termini from frame-shifted sequences could generate differences between proteins. In this context it also makes sense to explicitly define alignment gaps as mismatches (rather than i.e. being ignored in the identity count). Accordingly, also blastp from the NCBI BLAST+ package (e.g. version 2.9.0) is the tool of choice. It will create local alignments, typically ignore artificial N-termini and focus on the actually similar regions. Also, identity and similarity values are directly reported. These local alignments are more meaningful (in light of pseudo-genes and sequencing artifacts). However, if sequences are to be compared in their entirety (as-is, so to speak), clustal omega is the tool of choice.

Preferred variants of an epitope may be used that incorporate one or more point mutations in the epitope or epitopic region, such as at least 1, 2, 3, 4, up to 5 point mutations in the amino acid sequence, e.g. by insertion, deletion and/or substitution of an amino acid residue. Suitable point mutations are point mutations which are already present in other protein variants (as e.g. given in FIGS. 12B, 12C and 12D) and are then introduced in native proteins as derivatives according to the present invention with a different amino acid residue at this position.

Q6A5X9 and Q6A5P9 as reference sequences were compared to variants of both. This data shows the amino acid positions which are unique for CSD1, CSD2 and CSD3 in DsA1 compared to the respective CSD sequences in DsA2 (i.e. from which amino acid position it can be unambiguously concluded that a certain sequence is from CSD1 or CSD2 or CSD3 from DsA1 (or DsA2).

To compute percentages the count of a specific variant is compared against all sequences defined in this position. This can also include gaps, unless they are in the flanks. If the gaps are in the flanks, they should not be considered/counted as difference (they are a sign of the first sequence just being shorter/incomplete). A gap within a sequence is considered a difference.

E366D may be biased by C-terminally truncated translations, i.e. the process of generating match sequences after the genomic BLAST/search. Many of the BLAST derived sequences seem to lack the PT region (actually already CTPEPTPT, so slightly before the end of the CSD3), and this is probably a systematic technical issue. For this reason E366D as well as the baseline ‘E’ variant is very likely not correctly quantified, i.e. there may be arbitrarily more of either kind. Besides this reason for the truncation, the reason can also be of a technical nature or a sequencing issue.

The “alternative variant percentage” referred to hereinafter is the percentage of analyzed sequences which contains a specific variant not existing in the reference sequence. This analysis was done on the sequences dislclosed in FIG. 12 and the following, additional sequences: after EP priority date (including the version/status of the database):

GenBank entry ID (DsA1): VBYU0I000003.1:466799-467896 (22 May 2019) and QJIR01000003.1:466818-467915 (3 Jun. 2019), QJII01000011.1:3-1160 (3 Jun. 2019) and VBYK01000011.1:3-1151 (22 May 2019).

GenBank entry ID (DsA2): MVCC010003.1:313005-313898 (17 Oct. 2017), BFFM01000002.1:c62117-61161 (16 May 2019) and LKVC01000009.1:231840-232958 (16 May 2019), LKVC01000009.1 (16 Oct. 2017) and GCA_000145535.1_ASM14553v1 (2010/08/16) or GCA_000342585.1_PropiAcnFZ1_2_0_1.0 (2013/03/01).

The following intra-DsA1, intra-DsA2 and inter-DsA1-DsA2 amino acid variations are present in DsA1 and DsA2:

Position 50 of CSD1 of DsA1 (reference=D (7.6%) and variant=N (79.6%)) corresponds position 94 of DsA2 (reference=S (81.5%) and variant=D (10.9%)). Position 51 of CSD1 of DsA1 (reference=K (87.2%)) corresponds position 95 of DsA2 (reference=E (81.5%) and variant=A (10.9%)). Position 53 of CSD1 of DsA1 (reference=C (85.5%) and variant=Y (1.6%)) corresponds position 97 of DsA2 (reference=C (92.4%)). Position 55 of CSD1 of DsA1 (reference=D (87.2%)) corresponds position 99 of DsA2 (reference=K (92.4%)). Position 57 of CSD1 of DsA1 (reference=V (85.5%) and variant=1 (1.6%)) corresponds position 101 of DsA2 (reference=I (92.4%)). Position 61 of CSD1 of DsA1 (reference=A (87.2%)) corresponds position 105 of DsA2 (reference=L (92.4%)). Position 65 of CSD1 of DsA1 (reference=A (87.2%)) corresponds position 109 of DsA2 (reference=G (92.4%)). Position 68 of CSD1 of DsA1 (reference=A (87.2%)) corresponds position 112 of DsA2 (reference=V (92.4%)). Position 71 of CSD1 of DsA1 (reference=L (87.2%) and variant=M (2.5%)) corresponds position 115 of DsA2 (reference=L (92.4%)). Position 76 of CSD1 of DsA1 (reference=F (89.8%)) corresponds position 120 of DsA2 (reference=L (92.4%)). Position 78 of CSD1 of DsA1 (reference=S (89.8%)) corresponds position 122 of DsA2 (reference=A (92.4%)). Position 82 of CSD1 of DsA1 (reference=V (89.8%) and variant=M (10.1%)) corresponds position 126 of DsA2 (reference=A (92.4%)). Position 86 of CSD1 of DsA1 (reference=P (96.6%) and variant=S (2.5%) and variant=R (0.8%)) corresponds position 130 of DsA2 (reference=P (93.2%)). Position 90 of CSD1 of DsA1 (reference=K (20.3%) and variant=R (79.6%)) corresponds position 134 of DsA2 (reference=A (94.1%)). Position 94 of CSD1 of DsA1 (reference=K ((100%)) corresponds position 138 of DsA2 (reference=A (98.3%)). Position 97 of CSD1 of DsA1 (reference=V (100%)) corresponds position 141 of DsA2 (reference=T (98.3%)). Position 99 of CSD1 of DsA1 (reference=L (100%)) corresponds position 143 of DsA2 (reference=T (98.3%)). Position 100 of CSD1 of DsA1 (reference=I (100%)) corresponds position 144 of DsA2 (reference=I (84.8%) and variant=L (13.4%)). Position 104 of CSD1 of DsA1 (reference=K (100%)) corresponds position 148 of DsA2 (reference=R (98.3%)). Position 106 of CSD1 of DsA1 (reference=K (100%)) corresponds position 150 of DsA2 (reference=K (94.9%) and variant=E (3.3%)). Position 107 of CSD1 of DsA1 (reference=A (100%)) corresponds position 151 of DsA2 (reference=V (98.3%)). Position 109 of CSD1 of DsA1 (reference=I (100%)) corresponds position 153 of DsA2 (reference=V (98.3%)). Position 110 of CSD1 of DsA1 (reference=G (100%)) corresponds position 154 of DsA2 (reference=A (98.3%)). Position 111 of CSD1 of DsA1 (reference=A (100%)) corresponds position 155 of DsA2 (reference=S (98.3%)). Position 113 of CSD1 of DsA1 (reference=L (5%) and variant=V (94.9%)) corresponds position 157 of DsA2 (reference=L (98.3%)). Position 114 of CSD1 of DsA1 (reference=G (100%)) corresponds position 158 of DsA2 (reference=G (94.9%) and variant=S (3.3%)). Position 116 of CSD1 of DsA1 (reference=L (100%)) corresponds position 160 of DsA2 (reference=V (98.3%)). Position 117 of CSD1 of DsA1 (reference=T (100%)) corresponds position 161 of DsA2 (reference=A (98.3%)). Position 120 of CSD1 of DsA1 (reference=K (100%)) corresponds position 164 of DsA2 (reference=A (98.3%)). Position 121 of CSD1 of DsA1 (reference=1 (100%)) corresponds position 165 of DsA2 (reference=V (98.3%)). Position 123 of CSD1 of DsA1 (reference=R (100%)) corresponds position 167 of DsA2 (reference=H (98.3%)). Position 124 of CSD1 of DsA1 (reference=A (100%)) corresponds position 168 of DsA2 (reference=A (97.4%) and variant=T (0.8%)). Position 128 of CSD1 of DsA1 (reference=V (100%)) corresponds position 172 of DsA2 (reference=I (98.3%)).

From these sequences follows that the following CSD1 derivatives are preferred embodiments of the present invention, because of their conservative nature: from the perspective of CSD1 of Q6A5X9: a CSD1 derivative with one or more, preferably one, two or three, especially one, of the following amino acid exchanges:

D50N, D50S, K51E, K51A, C53Y, D55K, V57I, A61L, A65G, A68V, L71M, F76L, S78A, V82M, V82A, L84M, P86R, P86S, K90R, K90A, K94A, V97T, L99T, I100L, K104R, K106E, A107V, 1109V, G110A, A111S, L113V, G114S, L116V, T117A, K120A, I121V, R123H, A124T, V128I, in DsA1 and from the perspective of CSD1 of Q6A5P9: a CSD1 derivative with one or more, preferably one, two or three, especially one, of the following amino acid exchanges:

S94D, S94N, E95A, E95K, C97Y, K99D, I101V, L105A, G109A, V112A, L115M, L120F, A122S, A126M, A126V, L128M, P130R, P130S, A134K, A134R, A138K, T141V, T143L, I144L, R148K, K150E, V151A, V153I, A154G, S155A, L157V, G158S, V160L, A161T, A164K, V165I, H167R, A168T, I172V in DsA2.

Position 149 of CSD2 of DsA1 (reference=A (100%)) corresponds position 193 of DsA2 (reference=A (94.9%) and variant=T (3.3%)). Position 152 of CSD2 of DsA1 (reference=D (100%)) corresponds position 196 of DsA2 (reference=N (84.8%) and variant=S (13.4%)). Position 155 of CSD2 of DsA1 (reference=V (100%)) corresponds position 199 of DsA2 (reference=I (98.3%)). Position 163 of CSD2 of DsA1 (reference=V (100%)) corresponds position 207 of DsA2 (reference=I (94.9%) and variant=V (3.3%)). Position 166 of CSD2 of DsA1 (reference=K (100%)) corresponds position 210 of DsA2 (reference=H (97.4%) and variant=P (0.8%)). Position 168 of CSD2 of DsA1 (reference=A (98.3%) and variant=T (1.6%)) corresponds position 212 of DsA2 (reference=A (98.3%)). Position 169 of CSD2 of DsA1 (reference=K (100%)) corresponds position 213 of DsA2 (reference=R (98.3%)). Position 171 of CSD2 of DsA1 (reference=T (100%)) corresponds position 215 of DsA2 (reference=T (94.9%) and variant=A (3.3%)). Position 173 of CSD2 of DsA1 (reference=V (100%)) corresponds position 217 of DsA2 (reference=V (98.3%) and variant=M (1.6%)). Position 176 of CSD2 of DsA1 (reference=A (100%)) corresponds position 220 of DsA2 (reference=V (100%)). Position 183 of CSD2 of DsA1 (reference=A (98.3%) and variant=T (1.6%)) corresponds position 227 of DsA2 (reference=A (100%)). Position 190 of CSD2 of DsA1 (reference=T (100%)) corresponds position 234 of DsA2 (reference=F (95.7%) and variant=1 (4.2%)). Position 191 of CSD2 of DsA1 (reference=E (100%)) corresponds position 235 of DsA2 (reference=E (99.1%) and variant=K (0.8%)). Position 192 of CSD2 of DsA1 (reference=A (100%)) corresponds position 236 of DsA2 (reference=L (99.1%) and variant=F (0.8%)). Position 198 of CSD2 of DsA1 (reference=A (20.3%) and variant=G (79.6%)) corresponds position 242 of DsA2 (reference=A (100%)). Position 199 of CSD2 of DsA1 (reference=A (100%)) corresponds position 243 of DsA2 (reference=A (99.1%) and variant=T (0.8%)). Position 202 of CSD2 of DsA1 (reference=A (100%)) corresponds position 246 of DsA2 (reference=A (95.7%) and variant=T (4.2%)). Position 205 of CSD2 of DsA1 (reference=V (100%)) corresponds position 249 of DsA2 (reference=I (100%)). Position 206 of CSD2 of DsA1 (reference=G (6.7%) and variant=N (1.6%) and variant=S (91.5%)) corresponds position 250 of DsA2 (reference=R (84%) and variant=Q (15.9%)). Position 212 of CSD2 of DsA1 (reference=K (100%)) corresponds position 256 of DsA2 (reference=Q (100%)). Position 214 of CSD2 of DsA1 (reference=A (97.4%) and variant=T (2.5%)) corresponds position 258 of DsA2 (reference=A (100%)). Position 219 of CSD2 of DsA1 (reference=I (100%)) corresponds position 263 of DsA2 (reference=V (95.7%) and variant=A (4.2%)). Position 223 of CSD2 of DsA1 (reference=S (100%)) corresponds position 267 of DsA2 (reference=A (100%)). Position 225 of CSD2 of DsA1 (reference=D (100%)) corresponds position 269 of DsA2 (reference=D (99.1%) and variant=N (0.8%)). Position 233 of CSD2 of DsA1 (reference=V (100%)) corresponds position 277 of DsA2 (reference=I (100%)). Position 235 of CSD2 of DsA1 (reference=S (99.1%) and variant=F (0.8%)) corresponds position 279 of DsA2 (reference=S (100%)). Position 239 of CSD2 of DsA1 (reference=N (100%)) corresponds position 283 of DsA2 (reference=S (100%)). Position 255 of CSD2 of DsA1 (reference=L (100%)) corresponds position 299 of DsA2 (reference=I (100%)). Position 257 of CSD2 of DsA1 (reference=V (100%)) corresponds position 301 of DsA2 (reference=I (100%)). Position 258 of CSD2 of DsA1 (reference=Q (100%)) corresponds position 302 of DsA2 (reference=S (100%)). Position 259 of CSD2 of DsA1 (reference=I (100%)) corresponds position 303 of DsA2 (reference=L (100%)). Position 262 of CSD2 of DsA1 (reference=R (100%)) corresponds position 306 of DsA2 (reference=H (100%)). Position 264 of CSD2 of DsA1 (reference=I (100%)) corresponds position 308 of DsA2 (reference=V (100%)). Position 265 of CSD2 of DsA1 (reference=D (100%)) corresponds position 309 of DsA2 (reference=K (100%)).

From these sequences follows that the following CSD2 derivatives are preferred embodiments of the present invention, because of their conservative nature: from the perspective of CSD2 of Q6A5X9: a CSD2 derivative with one or more, preferably one, two or three, especially one, of the following amino acid exchanges: A149T, D152N, D152S, V155I, V163I, K166H, K166P, A168T, K169R, T171A, V173M, A176V, A183T, T190F, T190I, E191K, A192L, A192F, A198G, A199T, A202T, V205I, G206N, G206S, G206R, G206Q, K212Q, A214T, I219V, I219A, S223A, D225N, V233I, S235F, N239S, L255I, V2571, Q258S, I259L, R262H, I264V, D265K in DsA1 and from the perspective of CSD2 of Q6A5P9: a CSD2 derivative with one or more, preferably one, two or three, especially one, of the following amino acid exchanges: A193T, N196D, N196S, I199V, I207V, H210K, H210P, A212T, R213K, T215A, V217M, V220A, A227T, F234T, F234I, E235K, L236A, L236F, A242G, A243T, A246T, I249V, R250G, R250S, R250N, G250Q, Q256K, A258T, V263I, V263A, A267S, D269N, I277V, S279F, S283N, I299L, I301V, S302Q, L303I, H306R, V308I, K309D in DsA2.

Position 281 of CSD3 of DsA1 (reference=M (100%)) corresponds position 325 of DsA2 (reference=V (100%)). Position 283 of CSD3 of DsA1 (reference=N (100%)) corresponds position 327 of DsA2 (reference=D (100%)). Position 285 of CSD3 of DsA1 (reference=T (100%)) corresponds position 329 of DsA2 (reference=A (100%)). Position 286 of CSD3 of DsA1 (reference=R (100%)) corresponds position 330 of DsA2 (reference=R (95.7%) and variant=Q (4.2%)). Position 289 of CSD3 of DsA1 (reference=V (98.3%) and variant=A (1.6%)) corresponds position 333 of DsA2 (reference=V (100%)). Position 291 of CSD3 of DsA1 (reference=V (100%)) corresponds position 335 of DsA2 (reference=I (100%)). Position 292 of CSD3 of DsA1 (reference=I (100%)) corresponds position 336 of DsA2 (reference=R (100%)). Position 293 of CSD3 of DsA1 (reference=T (100%)) corresponds position 337 of DsA2 (reference=N (100%)). Position 294 of CSD3 of DsA1 (reference=A (100%)) corresponds position 338 of DsA2 (reference=T (100%)). Position 295 of CSD3 of DsA1 (reference=D (100%)) corresponds position 339 of DsA2 (reference-Q (95.7%) and variant=K (4.2%)). Position 296 of CSD3 of DsA1 (reference=K (100%)) corresponds position 340 of DsA2 (reference=E (99.1%) and variant=K (0.8%))). Position 298 of CSD3 of DsA1 (reference=I (100%)) corresponds position 342 of DsA2 (reference=I (72.2%) and variant=V (27.7%)). Position 299 of CSD3 of DsA1 (reference=K (100%)) corresponds position 343 of DsA2 (reference=A (100%)). Position 300 of CSD3 of DsA1 (reference=T (100%)) corresponds position 344 of DsA2 (reference=V (86.5%%) and variant=I (13.4%)). Position 301 of CSD3 of DsA1 (reference=A (100%)) corresponds position 345 of DsA2 (reference=Y (100%)). After position 301 of CSD3 of DsA1 the reference=gap (99.1%) and variant=D (0.8%) and this corresponds to a position after 345 of DsA2 that is a gap (100%). Position 302 of CSD3 of DsA1 (reference=E (100%)) corresponds position 346 of DsA2 (reference=K (100%)). Position 305 of CSD3 of DsA1 (reference=E (100%)) corresponds position 349 of DsA2 (reference=K (100%)). Position 306 of CSD3 of DsA1 (reference=K (100%)) corresponds position 350 of DsA2 (reference=A (100%)). Position 310 of CSD3 of DsA1 (reference=A (100%)) corresponds position 354 of DsA2 (reference=T (100%)). Position 313 of CSD3 of DsA1 (reference=D (100%)) corresponds position 357 of DsA2 (reference=G (100%)). Position 316 of CSD3 of DsA1 (reference=K (100%)) corresponds position 360 of DsA2 (reference=Q (100%)). Position 320 of CSD3 of DsA1 (reference=S (100%)) corresponds position 364 of DsA2 (reference=T (99.1%)). Position 321 of CSD3 of DsA1 (reference=C (100%)) corresponds to a gap (100%) at DsA2. Position 322 of CSD3 of DsA1 (reference=P (100%)) corresponds position 365 of DsA2 (reference=P (97.4%) and variant=L (1.6%)). Position 323 of CSD3 of DsA1 (reference=K (100%)) corresponds position 366 of DsA2 (reference=E (86.5%) and variant=D (12.6%)).

From these sequences follows that the following CSD3 derivatives are preferred embodiments of the present invention, because of their conservative nature: from the perspective of CSD3 of Q6A5X9: a CSD3 derivative with one or more, preferably one, two or three, especially one, of the following amino acid exchanges:

M281V, N283D, T285A, R286Q, V289A, V291I, I292R, T293N, A294T, D295K, D295Q, K296E, I298V, K299A, T300V, T300I, A301 Y, gap between 301 and 302 D, E302K, E305K, K306A, A310T, D313G, K316Q, S320T, C321—, P322L, K323D, K323E in DsA1 and from the perspective of CSD3 of Q6A5P9: a CSD3 derivative with one or more, preferably one, two or three, especially one, of the following amino acid exchanges:

V325M, D327N, A329T, R330Q, V333A, I335V, R336I, N337T, T338A, Q339K, Q339D, E340K, 1342V, A343K, V344T, V344I, Y345A, gap between 345 and 346 D, K346E, K349E, A350K, T354A, G357D, Q360K, T364S, gap between 364 and 365 C, P365L, E366D, E366K in DsA2.

Variants include, for instance, proteins wherein one or more amino acid residues are added, or deleted, at the N- or C-terminus, as well as within one or more internal domains. Specific variants as described herein comprise additional amino acids at the N-terminal and/or at the C-terminal end, to prolong an antigen sequence as described herein, e.g. to prolong a sequence of an epitope or epitopic region within a protein by at least one amino acid residue, preferably by less than 3 amino acids, specifically less than 5, or else less than 10 amino acids. Further variants may be fusion proteins, wherein an antigen sequence as described herein is prolonged by additional amino acid residues of another polypeptide or protein. According to a preferred embodiment, the vaccine according to the present invention comprises a derivative, especially as a fusion protein, wherein a DsA1/DsA2 fragment containing a DsA1/DsA2 epitope and/or a PITP fragment containing a PITP epitope is prolonged by additional amino acid residues of another polypeptide or protein, preferably by one or more immunologically relevant epitopes, especially wherein the derivative comprises a His-tag at the N- or at the C-terminus, comprising at least 4, preferably at least 5, especially at least 6 histidine residues. In general, His-tags may comprise at least two to 10 or more histidine residues. His tags are preferably included in derivatives which are intended for experimental purposes and not necessarily included in the vaccine intended for human use. Accordingly, any sequence with a His-tag used herein shall also be regarded as being disclosed without the His-tag.

Variants include, for instance, proteins wherein one or more cysteins have been exchanged which results in reduced product related impurities and microheterogeneities, improved protein stability, folding or other types of biochemical properties (FIG. 10b, Table 2).

According to a preferred embodiment, the derivatives according to the present invention are “Cys-replacement derivatives” wherein one or more of the naturally occurring cysteine residues are replaced by a different amino acid residue. For example, the following cysteines may be exchanged: DsA1: C53, C319, C321; DsA2: C97, C363 (there are even more cysteines in the N-terminus of some DsA2 proteins which may be exchanged; see e.g. SEQ ID NO:8); PITP: C231, C402, C460. Of course, the absolute amino acid numbering in a given polypeptides (e.g. in a fragment or derivative) changes with the length of the fragment/derivative; this numbering of cysteines (DsA1: C53, C319, C321; DsA2: C97, C363; PITP: C231, C402, C460), however, allows an absolute identification of the cysteines to be exchanged. For example, in DsA1 fragments/derivatives in the sequence listing, the cysteines C53. C319 and C321 correspond to C53, C325 and C327 in SEQ ID NO:4; C71, C337 and C339 in SEQ ID NO:5; C26, C292 and C294 in SEQ ID NO:7; C119 and C121 in SEQ ID NO:31; C56 and C58 in SEQ ID NO:32; C53, C325 and C327 in SEQ ID NO:34; C231 and C233 in SEQ ID NO:35; C38 and C304 in SEQ ID NO:36; C32, C298 and C300 in SEQ ID NO:37; C32 in SEQ ID NO:38; etc. In DsA2 fragments/derivatives in the sequence listing, the cysteines C97 and C363 correspond to C67 and C333 in SEQ ID NO:8; C97 and C363 in SEQ ID NO:9; C74 and C340 in SEQ ID NO:10; C33 and C299 in SEQ ID NO:11, C27 and C293 in SEQ ID NO:12; etc. In PITP fragments/derivatives in the sequence listing, the cysteines C231, C402, C460 correspond to C201, C372 and C430 in SEQ ID NO:15; C201 in SEQ ID NO:17; C372 in SEQ ID NO:18; C201 in SEQ ID NO:19; etc.

The exchange is preferably made by an amino acid which is similar in size and charge/polarity as cysteine, however, without any sulphur groups. It follows that exchange of one or more cysteines are preferably not methionine, arginine, histidine, lysine, tryptophan, aspartic acid or glutamic acid. Preferred exchanges are therefore exchanges of cysteine(s) with serine, proline, alanine, threonine, asparagine, glutamine, valine, isoleucine, leucine, phenylalanine, tyrosine and glycine, preferably with serine, proline, alanine, threonine, asparagine, glutamine, valine, isoleucine, leucine, especially with serine, proline, alanine, threonine, asparagine, and glutamine. In a given derivative of DsA1, DsA2 or PITP or of a fragment thereof, preferably at least two cysteines are exchanged, exchange of two cysteines in each of these fragments/derivatives is specifically preferred.

A preferred embodiment of the present invention is a derivative of DsA1 wherein C53 is exchanged, especially wherein a C53S exchange is present (such as in SEQ ID NOs:48 and 49). Another preferred embodiment of the present invention is a derivative of DsA1 wherein C319 is exchanged, especially wherein a C319S exchange is present. Another preferred embodiment of the present invention is a derivative of DsA1 wherein C321 is exchanged, especially wherein a C321P exchange is present. A specifically preferred embodiment of the present invention is a derivative of DsA1 wherein two cysteines are exchanged, preferably wherein all three cysteines are exchanged, especially wherein a C53S, a C319S and a C321P exchange are present. Another preferred embodiment of the present invention is a derivative of DsA2 wherein C97 is exchanged, especially wherein a C97S exchange is present. Another preferred embodiment of the present invention is a derivative of DsA2 wherein C363 is exchanged, especially wherein a C363S exchange is present. Another preferred embodiment of the present invention is a derivative of PITP wherein C231 is exchanged, especially wherein a C231S exchange is present. Another preferred embodiment of the present invention is a derivative of PITP wherein C402 is exchanged, especially wherein a C402S exchange is present. Another preferred embodiment of the present invention is a derivative of PITP wherein C460 is exchanged, especially wherein a C460S exchange is present.

Other preferred DsA derivatives according to the present invention may however, also be designed in the other direction, i.e. derivatives wherein two or more amino acid residues in a fragment or a derivative are changed to a cysteine (Cys, C) residue to further stabilise the fragment or a derivative by the ability to form disulfide bonds, preferably (with respect to the DsA1 sequence (which is also applicable to the DsA2 sequence; FIG. 12A) containing at least one of the following amino acid pairs wherein the native amino acid is exchanged to cysteine: PHE150-GLY185, LEU167-ALA199, ASN157-ALA192, LYS166-ALA199, ALA153-GLY 185, ASP164-ALA199, VAL154-ALA179, PHE150-LEU184, VAL154-ALA188, ALA153-ALA188, ALA179-GLU191, THR178-GLU19I, ALA161-ALA195, ILE175-ALA195, ILE175-GLU191, LEU158-ALA176, ASN157-ALA188, ASN157-ALA179, ASP164-GLU196, VAL154-LEU180, ALA153-LYS189, ASN157-LYS189. ALA161-ALA192, LEU167-ALA198. LEU158-ALA179, ALA160-ALA192, LYS165-ALA199, LYS166-GLN200; VAL205-ALA260, ILE232-ARG266, SER235-ILE263, SER235-ARG262, ILE232-LEU267, PR0236-ILE263, ALA231-ARG266, ASN239-ILE259, ILE232-ILE263, VAL209-PRO236, ALA201-ALA256, SER235-ILE259. VAL228-ARG266, ALA202-ALA256, ALA201-VAL257, GLY206-VAL243, GLY208-ILE264, GLY208-ILE263, VAL205-ASN239, LEU238-ILE259, GLY208-ALA260, ALA201-LYS253, ALA202-VAL243, LEU204-ALA260, VAL205-ALA256, VAL205-ILE259, SER235-ARG266, ASN239-ALA260, VAL209-ILE263, or VAL209-ASN239.

Specifically preferred cysteine-replaced fragments and derivatives of PITP comprise at least or consist of the following PITP fragments: A32 to R164, A32 to Q198, A32 to T143, A32 to V148, A32 to T171, P34 to R164, A32 to T159, A32 to I177, A32 to Q204, A32 to G234, A32 to K400, A32 to S391, V238 to K400, A199 to T430, V238 to T395, G234 to K400, H223 to K400, T232 to V401, V238 to T392, V238 to N393, V238 to L394, V238 to T395, V238 to L396, T232 to T430, G172 to K400, and G172 to G234, especially the fragments A32-T430, A32-I467, A32-S391 (with K174-T239 deleted), A32-S391, with a replacement of at least one of C231, C402, and C460 is exchanged, preferably wherein of at least two of C231, C402, and C460 are exchanged, especially wherein C231 and C402 are exchanged. Preferably, the exchanges of cysteine are also with serine, proline, alanine, threonine, asparagine, glutamine, valine, isoleucine, leucine, phenylalanine, tyrosine and glycine, preferably with serine. Accordingly also for these fragments and derivatives, presence of the C231S, the C402S, and/or the C460S exchange is preferred, especially the C231S and the C402S. These fragments contain at least one efficient epitope to elicit an appropriate, cross-type reactive immune reaction if provided as a vaccine in humans. Moreover, these fragments are efficiently producible by recombinant expression systems and finishable in final pharmaceutical vaccine formulations.

Besides the cysteine exchanges, other amino acid exchanges which enable higher stability, increased solvation and/or increased pH stability of the polypeptides are preferred in the derivatives of the present invention. For example specific lysine to arginine exhanges can stabilize unstructured regions or further stabilize already structured regions in a polypeptide (the latter being specifically preferred as this can further stabilize already structured epitopes). Amino acid residues with side-chain solvent accessibility of no more than 20% may be exchanged (preferably by polar or charged amino acid residues, especially aspartic or glutamic acid, lysine or arginine residues, to increase stability and solubility as long as these exchanges do not have a detrimental effect on the antigenicity profile.

After having identified the epitopes of the polypeptides according to the present invention, amino acid exchanges of the polypeptide sequences were analysed based on the behavior of these polypeptides at varying pHs with residues that exert a de-stabilizing effect (referring to buried acidic residues). For example, exchange of acidic residues focuses on buried residues, as these destabilize at higher pH.

The exchange specifically of histidine to lysine focuses on solvent-accessible residues and solvation. It is differentiated between stability and solubility, as exchanges can affect the one, the other or both, and sometimes in a tradeoff. Biochemically, a mix of the two was observed, but that is mostly a question of assays used. I.e. there can be pH driven insolubility (which may in turn lead to complex formation and possibly loss of structure through hydrophobic interactions when protein concentration is too high), but also loss of structure due to temperature or denaturing agents, which may also lead to complex formation (although not necessarily) because hydrophobic parts get exposed.

Preferred exchanges were provided which also have a stabilizing effect extending the one by reducing buried acidic residues. Exchanges which benefit overall stability in addition to pH dependent stability are specifically preferred. pH stability may be determined e.g. by the Prometheus technique (Chattopadhyay et al Prot. Sci. 28 (2019). 1127-1134; Martin et al., 2014-NanoTemper Technologies GmbH-Application_Note_NT-PR-001_-_Thermal_Unfolding: Krakowiak et al., J. Biol. Phys. 45 (2019), 161-172) using differential scanning fluorimetry (see example section). Differential scanning fluorimetry allows the measurement of thermal unfolding or chemical denaturation under native and label-free conditions by detecting changes in protein intrinsic fluorescence during a thermal ramp or in the presence of a chemical denaturant. For thermal unfolding, the temperatures of the transitions from the folded to the unfolded state are determined. Higher transition temperatures correspond to higher thermal stability.

As a stabilizing effect, the proportion of acidic residues in the hydrophobic core is reduced and the proportion of charged residues in the (solvent accessible) periphery is increased, but mostly it is important to spread charge and therefore solvation across the surface. The first aspect protects against denaturation (as there is no major pH driven benefit from solvating hydrophobic residues), while the second aspect improves solubility.

Accordingly, preferred derivatives of the DsA1 polypeptides and fragments of the present invention contain the following amino acid exchanges:

A301Y, G206L, G206F, A192L, G206R, G206W, G206Q, G206K, A301F, A301L, G206I, A301W, A301I, G206E, G206M, G208L, G206V, A192M, A256L, A192I, A68I, G206N, A68V, I259L, A161L, V205I, G206T, G206H, A68L, G206Y, A2561, G206A, 68M, A192F, G110A, N239L, G206C, A301V, E305L, A93I, A93L, S223A, E305I, E305V, E305A, K120V, K120L, N157I, K120C, E305C, E305M, N239I, K120I, R248C, N239V, E191I, E305F, S78I, K120M, E191L, R248L, H146V, E191V, H1461, S78L, S78V, N157V, H146C, K120F, R248I, R248M, D164L, P236V, P236L, H146L, H146F, D164I, H146M, P86L, R248G, N239C, N157C, E305T, E191C, S78C, R248F, G221L, E305G, P236F, T171L, P86V, R248S, N157L, K120A, H146A, P861, N157F, S235A, R248A, E191M, N157M, A124I, P236A, S78F, P236C, S78M, A161I, N239M, T171I, E191F, S235V, P86F, H146G, S223I, E305S, D164F, E305Y, P236M, S223L, N239F, H146Y, P236I, G221V, D164C, K120G, D164M, S235I, R248V, A124V, S235L, S223V, S147L, S235C, P86M, G208I, D164A, S1471, N239A, K120T, T117I, G110L, R248Y, P182I, A198L, A124L, A215L, A127L, A66L, P86C, G221F, R248T, T171F, P182V, S147V, P271V, T171V, G110V, P271A, E191A, A149L, K120Y, G114I, K120S, S235F, A201I, G131V, A201V, R248N, A66V, A202I, G221C, 114V, A229L, H146T, A124F, A127F, T117V, S223C, A215F, A127I, P182C, A215V, G162L, G162I, G208F, A201L, A161F, N157A, A198V, E191Y, A161V, S223F, G221M, R248W, G131C, A202V, N157T, S147F, N157Y, G114A, T171C, H146W, S78T, A149I, G250L, N239T, A149V, G208C, K120P, G250V, S235G, S223M, D164V, N239G, T171M, E191G, S78A, D164Y, P271I, G110F, G110C, D164W, P236Y, A124C, A66F, S147M, G75L, E305N, S147C, G221I, A127M, P182L, G221A, G110M, A66C, E305Q, P271F, A215M, P236W, K120W, H146P, A161C, A229I, S78G, G114M, P86A, E305W, V154L, G185V, G75I, S223Y, G75V, R248Q, T277V, E191S, A229F, T277G, G131I, N239S, P271C, V154I, A66M, D164G, G208A, P236T, R248D, D164T, A127C, A215I, A161M, P86W, A201F, C53L, T117F, G131A, T117C, A318L, F150L, G208M, P271M, A149C, G208V, G114C, G162F, S223W, A198I, N1570, A202L, P2360, G185I, A198F, A149M, P86Y, E191W, N157W, I263L, S235T, E305P, E305R, A201C, A201M, F150I, G96I, G114F, G221Y, A199L, D164S, A124M, A179I, A202F, H146S, A231L, C53I, I263V, G162M, K120Q, N239Y, G96L, G75C, N157S, T277C, T117A, A199V, G162C, P271Y, G250C, A127Y, A66I, T117L, G185C, A229C, R248P, A144I, T117M, P86T, A260L, G185A, E191Q, G131L, T277I, G250I, G131M, T277A, A127V, F150V, A318I, S78W, S147Y, A198M, P86G, A215C, A701, S235M, A179V, A202M, A144V, A202C, G208W, T171Y, S147W, G208Y, G250F, A195L, K120H, A127W, A66Y, S223G, K120N, S78Y, E191N, A198C, P2710, V154F, A70V, H146N, A229M, A134L, P86S, S78Q, A199I, G75M, A68C, E191T, A89L, G96A, D164P. C53V, S78P, G185L, A149G, A149F, and P182A (which are preferred exchanges with stabilizing effect);

D265K, D55K, K169R, H103D, H102D, H102E, H103E, D313E, D312E, K316R, K104D, K212E, D312K, K212D, K166D, K212R, K317D, K166E, K317E, K317R, K213R, D312R, H103R, K316E, K165E, H102R, D313K, K213E, D313R, A126K, K213D, K166R, A126E, K165R, K104E, H245E, K282E, N283E, A126R, S77K, P142R, H102K, H245D, K316D, A125K, S77R, A126D, A138K, P142K, K133D, S234E, H210E, P261K, P142E, D129E, H210D, A138E, A227K, A138R, H218K, D60E, D80R, and D80K (which are preferred exchanges with increased solvation).

Accordingly, preferred derivatives of the DsA2 polypeptides and fragments of the present invention contain the following amino acid exchanges:

A161L, A161I, A126I, A126V, A126L, S175G, G190H, A161T, S183T, R336I, A126F, A161F, A329L, A161V, S283L, A205L, G109A, A329I, G265F, A161W, A2321, S175W, R336L, A300I, K349I, A161M, A300L, G109L, G109I, A246L, S183M, A223L, E235V, G252L, A329M, A126W, S183L, A259L, A329V, A329F, S183I, A242L, G109F, R336V, K117I, R336C, K117C, K349V, R336A, R336F, K117L, R336M, E235I, R310A, K349L, K117V, R310V, K349C, N201I, R310I, R310C, K349F, K349M, K117M, R292C, N201V, R336G, R292L, K117F, E235C, P130L, E235A, D208L, P280L, E235L, R292M, R336T, P280V, R292I, N201L, E235M, P130I, K117A, K349A, P130V, R292G, R310F, R336S, R336Y, N201C, K117G, R310L, R310M, R310G, R336W, D208V, R292F, P130F, K349Y, K117T, R310T, P280C, R292S, N201F, R310S, T215L, N201M, S279C, R292A, S279L, P280F, D208C, S175V, A126M, P280A, S279A, P130C, S279V, E235F, P186C, P130M, K349T, A126C, K349G, N320V, K117S, N320I, S283V, A168I, N320L, S175I, E235Y, P186V, P280I, P186A, P280M, G265L, D208M, P186L, K117Y, S283I, A205I, D208F, A168L, T215I, R292V, A133L, R336P, R310Y, A168V, S279F, S183V, A164I, S279I, S175L, D208A, K349S, E235T, S175C, A122I, D208I, A126Y, A123L, K117P, N320C, R292T, S283C, E235W, G252I, T215V, R292Y, R336Q, S175A, A123I, E235G, A205V, A171V, A133V, A245I, S183C, P226V, R310P, G181I, P226I, A168C, A246I, A171L, N201A, S175M, G158I, A164L, T215C, A164C, P280W, A133C, G119I, T215F, A245V, N320M, A122V, A123V, G206L, N320A, G265V, A122L, A193L, S191V, R310W, G252F, G181V, E235S, R292N, P130W, G119V, G265C, S279M, A259V, A259F, A168F, A168M, A246V, P226C, R292W, G158V, P130Y, G206V, P186G, C97L, N201Y, A171C, G265M, G140L, K117N, G140I, D208G, A193V, G181F, K349W, S279G, A245L, A205F, G158A, A193I, K117W, A122C, R310D, G294V, P186T, S175F, S191C, N201T, K349Q, R310N, A242V, T215M, D208Y, T321C, G252C, I276L, A259M, A171F, N201G, G119F, D208T, P186M, G229V, G294L, S191I, P186S, G119C, G2291, P130A, A205C, A171M, T321V, A193C, F268L, A133M, A123F, G357A, G009V, K117Q, N3200, R310Q, T321G, S283M, P186Y, G229A, R336D, N201S, S183F, F194L, F268V, A245F, G265A, G252A, R336H, A259I, A133I, A123C, P130T, G113L, D208S, P280T, F268I, G181C, P280Y, R336E, P280G, G158C, N320Y, G140V, K349P, R292D, D208W, N320F, G265I, R336N, A164M, G206M, F268C, G158M, R292P, S191L, G158F, G252M, A245C, G229C, E235Q, G252V. A205M, G206F, R292Q, G181M, A126G, A243L, K349N, S283A, A246M, S279T, P186I, A246F, A246C, A223I, C97I, A164V, A245M, A171I, G357I, A126T, S283F, A123M, F194I, K117D, P226L, P130S, G140F, I276F, F194V, A243V, P130G, G181L, G229F, A259C, G113I, K117H, N320S, G119M, A239L, G206C, A114I, E235N, K349D, A242I, A161C, G294C, G119L, A126S, T321L, A193M, A223V, A122M, G113C, A242C, N201W, R292H, A275L, A242M, G357V, P186F, G294I, D208P, A259W, A243I, R310E, G140C, A242F, G265W, S183A, G109C, G113F, S191F, and G265Y (which are preferred exchanges with stabilizing effect);

S94D, E95K, A343K, A350K, F176D, N196D, H147D, H146D, H147E, H146E, T91E, R250E, T91D, R251E, R148D, H254E, A170K, R250K, R251K, H254D, A170R, H146K, H147K, K346E, T90D, R250D, T90E, A138K, R251D, H262K, A169K, H146R, I102E, T90K. A271K, K346R, H306E, D124E, K326E, A169R, D124R (which are preferred exchanges with increased solvation).

Accordingly, preferred derivatives of the PITP polypeptides and fragments of the present invention contain the following amino acid exchanges:
T392F, F352L, T392I, M303F, A276Y, S365I, T392L, M303W, A276L, T392W, K363T, A361V, F286Y, A361I, K363F, M303Y, T392V, A361L, S365V, A270V, A276M, S365L, K363V, M312L, T252L, M387L, Y297F, A276F, G353T, A276W, K363S, T252R, L314I, T252I, A361F, L295I, L255I, A276I, K363L, S318W, V354L, K363I, A361T, S318I, A361M, G353L, T392Y, S365A, A270I, A361W, T252M, K363A, K363W, L319I, G353D, A276V, G353N, G353F, T252V, G353S, S318V, L255V, G364I, L394V, K363M, S318F, L390I, G353I, S335Y, G364L, S365M, T252Q, S365F, L330A, T252F, S318M, K363Y, G353V, S365G, S365W, K363G, T392M, V354F, A276T, G364W, K363C, P3171, S335C, P317L, P317F, G269C, S335A, P317V, G268C, S334V, S335V, G269V, S334C, A361C, G268I, G269F, G268L, S334I, G268V, G269L, S335G, S334A, G268F, S335T, P317C, P317M, S335L, A361Y, G269I, S335I, G268Y, K363P, G268W, S335M, K363H, F286I, G268M, G269M, G269W, G268A, S334G, S334L, G268P, G268T, G293F, F286L, T252C, S335N, T392C, K363N, S334F, M303I, K363Q, A376L, G364V, W245I, G269S, Y297C, S335Q, G358V, W245L, A376I, S334M, G358L, T252A, S318C, S335H, Y297L, S365C, M312I, G373L, Y297I, G358I, S335F, G268N, M303L, G269T, Y297V, P317A, G268Q, G268H, G293L, S334T, G305C, S318L, S335D, S335P, G364C, W245V, W245F, P317Y, T392A, F286V, S335W, A376V, G358C, K363D, M312F, G269N, S335R, A378I, A378C, P317W, G268K, G268R, G268D, A376C, G373V, M312V, M303V, G353C, S318A, G364F, G241C, G268E, G379I, G269P, F286C, L330F, G284V, W245C, A378V, L330I, G293I, G373C, G379L, S318Y, G358F, L394I, L330V, G373I, F352I, A361G, S335K, G246C, M387F, A378L, G241I, V354C, M387I, G269A, G308C, F352C, A276C, G293C, G269Q, K363R, G379V, F286W, W245M, G246V, A270C, G246L, M303C, G293V, G305A, Y297A, G345I, G241V (which are preferred exchanges with stabilizing effect);
N388D, R248K, S336D, T372E, T249E, E316D, D348E, R382E, R382K, K265E, H327K, D338E, K333R, P386E, S336E, K341D, K341E, K341R, E385D, G268K, G268R, G268D, K340R, G268E, K333D, K368R, G353D (which are preferred exchanges with increased solvation);

H306I+S335R, H306L+S335R, H306I+S335K, H306C+S335R, H306C+S335R, H306I+S335K, H306L+S335K, H306L+S335K, H306L+S335R, H306V+S335R, H306I+S335R, H306V+S335R, H306C+S335K, H306F+S335K, H306V+S335K, H306V+S335K, H306F+S335R, H306F+S335R, H306W+S335R, H306F+S335K, H306M+S335R, H306W+S335K, H306W+S335R, H306M+S335K, H306A+S335R, H306A+S335R, H306W+S335K, H306A+S335K, H306Y+S335R, H306Y+S335R, H306Y+S335K, H306T+S335R, H306Y+S335K, H306T+S335R, H306T+S335K, H306S+S335R, H306T+S335K, H306S+S335K, S334V+S335R, H306S+S335K, S334V+S335K, S334I+S335R, S334C+S335R, H306P+S335R, M312I+S335R, S334I+S335K, S334V+S335R, S334V+S335K, H306P+S335K, S334C+S335R, S334A+S335R, M312F+S335R, M312L+S335R, S334C+S335K, H306R+Y381L, M312L+S335R, S250L+S335R, H306Q+S335R, H306N+S335K, S334C+S335K, S334A+S335K, H306P+S335K, H306Q+S335R, M312V+S335R, M312I+S335K, S334I+S335K, M312I+S335R, M312L+S335K, H306N+S335R, S334L+S335R, S334A+S335K, M312L+S335K, S2501+S335R, H306N+S335R, S250V+S335R, H306N+S335K, M312F+S335K, S250V+S335R, S250A+S335R, S334M+S335R, M312V+S335R, S250A+S335K, M312F+S335K, S250A+S335K, S250L+S335R, S334G+S335R, S250C+S335R, S250C+S335R, H306Q+S335K, S334G+S335R, Y254F+S335R, S334L+S335R, S250I+S335R, I309C+S335R, S250I+S335K, Y254C+S335R, S250L+S335K, M312V+S335K, S334G+S335K, S250L+H306R, S250V+H306R, H306R+Y381I, M312V+S335K, S2501+H306R, M312C+S335R, Y254C+S335R, S250V+S335K, Y254W+S335R, S334G+S335K, S250L+H306R, S250L+S335K, Y254W+S335R, S250V+S335K, S334L+S335K, I309C+S335R, I309C+S335K, Y254F+S335R, S250A+S335R, S250C+S335K, S2501+H306R, S250A+H306R, S250C+S335K, H306Q+S335K, Y254C+S335K, S334L+S335K, S250A+H306R, I309C+S335K, Y254W+S335K, Y254C+S335K, Y254I+S335R, H306R+Y381C, S250M+S335K, M312C+S335K, M312C+S335K, S250V+H306R, S250M+S335R, Y254V+S335R, Y254I+S335K, S250W+S335R, I259C+S335R, S334M+S335R, Y254A+S335R, Y254V+S335R, S250M+S335K, F251L+S35R, Y2541+H306R, I259C+S335K, Y254I+S335R, I259C+S335K, S250Y+S335K, Y254F+S335K, S250M+S335R, Y254F+H306R, Y254V+S335K, Y254V+H306R, S334T+S335K, S334Y+S335R, Y254A+S335R, S334M+S335K, Y254I+H306R, Y254M+S335K, Y254A+S335K, S334F+S335R, S334M+S335K, F251L+H306R, S334T+S335R, Y254I+S335K, Y254A+S335K, S334T+S335K, S250Y+H306R, Y254A+H306R, Y254M+S335K, F251L+S335K, I259C+S335R, F251L+H306R, Y254M+H306R, S334P+S335R, S334P+S335R, S334P+S335K, S334F+S335R, Y254M+S335R, H306I+S335C, H306L+S335C, H306L+S335C, H306I+S335C, H306V+S335C, H306C+S335C, H306I+S334V, H306V+S335C, H306C+S335C, H306I+S334V, H306L+S335V, H306M+S335C, H306I+S335G, H306I+S335G, H306I+S335A, H306I+S335A, H306F+S335C, H306F+S335C, H306L+S335V, H306I+S335T, H306L+S335A, H306C+S335A, H306I+S335V, H306I+S335V, H306L+S334V, H306L+S335A, H306C+S335V, H306L+S334C, H306I+S335I, H306I+S334A, H306L+S334C, H306L+S334V, H306F+S334V, H306C+S335A, H306F+S335V, H306C+S334V, H306C+S334V, H306I+S335L, H306C+S335V, H306L+S334I, H306M+S335C, H306I+S334C, H306I+S335I, H306W+S335C, H306V+S335A, H306I+S334C, H306I+S334I, H306L+S335I, H306V+S335A, H306C+S334C, H306L+S335G, H306C+S334C, H306L+S335T, H306I+S334I, H306V+S334V, H306I+S335L, H306M+S335V, H306V+S335V, H306V+S335V, H306I+S335M, H306L+S335T, H306V+S335I, H306C+S335G, H306L+S335L, H306C+S334I, H306L+S335Y, H306L+S335I, H306V+S334V, H306C+S335G, H306I+S334A, H306L+S334A, H306C+S335I, H306F+S335V, H306I+S335T, H306L+S335Y, H306C+S334I, H306L+S334I, H306V+S335G, H306V+S335G, H306A+S335C, H306A+S335C, H306M+S335A, H306C+S334A, H306V+S335I, H306L+S335G, H306L+S334A, H306C+S334A, H306I+S335Y, H306I+S335Y, H306W+S335C, H306F+S335A, H306F+S335A, H306F+S335I, H306I+S335N, H306L+S335L, H306I+S335M, H306I+S335N, H306V+S334I, H306V+S334C, H306V+S335L, H306C+S335L, H306C+S335L, H3061+A376L, H306F+S335T, H306L+S335M, H306F+S334V, S334V+S335C, S335C+Y381L, S335C+A376I, S334I+S335C, S334V+S335C, M312I+S335C, M312I+S335C, S335C+A376L, S334I+S335C, S335C+A376L, S335C+A378C, S335C+A376C, S335C+A376V, S334A+S335C, S335C+A378C, S334A+S335C, S335C+A376I, S334V+S335A, S335A+A376L, S335C+A376C, S335C+A376V, S335C+A378L, S334V+S335A, S335A+A376I, S334V+A376L, S334I+S335V, S335C+A378I, M312F+S335C, M312F+S335C, S334I+S335A, S335C+A378V, S335V+A376L, S334V+S335L, S335C+A378I, S334C+S335V, S334V+A376I, S334V+A376I, S335V+A376I, M312I+S334V, S334V+S335I, S335C+Y381I, M312V+S335C, S335C+A378L, S335A+Y381L, S334C+A376C, S334V+S335I, S335C+Y381V, M312L+S335C, S335C+A378V, S335V+A376L, S334I+S335V, S335C+Y381C, S335V+A376I, S334C+A376L, S334C+Y381L, M312V+S335C, S334V+S335L, S334C+A376I, S334C+A376C, S334V+Y381L, M312L+S335C, S335V+A376V, S334I+A376I, S335V+A376V, S335V+Y381L. Y254F+S335C, L310F+S335C, S335I+A376I, S334V+A376V, M312I+S335V, S334C+S335A, S3341+A376L, M312F+S335V, S335C+Y381A, L310F+S335C, S334G+S335C, S334G+S335C, S334V+A376V, S335C+A376M, S335C+A376M, S334A+S335I, S334V+A376L, M312C+S335C, S334C+S335A, S335A+A376V, S334A+S335V, S334A+S335V, Y254F+S335C, S335A+A376L, S335A+Y381I, S335A+A376I, S334L+S335C, S335A+A376V, and M312C+S335C (which are preferred exchanges with increased solvation and/or reduced porphyrin binding).

These mutations are ranked by their assumed ability to stabilize domains and proteins against temperature induced denaturation (primarily heating) as well as denaturating agents (such as urea).

The amino acid exchanges as disclosed herein may also be combined and also shuffeled in derivatives which contain fragments of DsA1, DsA2 and PITP. Based on the illustrative example of the H4 polypeptide (e.g. SEQ ID NO: 49), the following amino acid exchanges are preferred to increase stability and/or solvation in a DsA1/DsA2/PITP derivative comprising at least one P. acnes epitope of SEQ ID NO: 49 (in the following paragraph, amino acid numbering is according to SEQ ID NO: 49 and not, as elsewhere, according to Q6A5X9, Q6A5P9 and Q6A9N1):

A274Y, S26C, S26L, A274F, 526I, G119H, A274L, A274W, S26V, S26M, S212L, T108I, A134L, S26F, N130V, A229L, A274I, G194F, S26W, A291F, 0181I, A229I, E278I, A175L, G181L, A152L, A274V, N130L, E278V, K46I, E278L, E278A, K46C, K46L, K93L, K93V, E164I, E278C, E164V, E278M, N130I, K46V, R221C, K93C, K46M, S51L, K93I, E278F, K93M, E164A, S51I, R221L, P209L, K46F, S51V, E164C, R221I, E164M, K93F, D137L, P209V, E164L, P59L, R221F, N130C, S51C, R221G, E278T, K46G, P59V, K46A, N130M, E278G, D137V, K46T, P209C, P59I, P209I, T144L, N130F, E164F, S51F, R221M, P209F, S51M, R221S, P209A, K93A, P209M, E164Y, D137C, R221A, P115C, P59F, S208A, K46S, P115V, A134I, P115A, S212V, N249L, E278S, D137F, D137M, P115L, S212I, R221V, N249V, D137I, E278Y, G194L, P59M, R221T, A188L, N249I, T144I, E164T, S208C, S208V, D137A, K46Y, K93G, G87I, E164G, K46P, P59C, A97V, S212C, G83L, K93T, N249C, A134V, G83V, T108V, T112L, T144V, A100L, G87V, E164W, E164S, A171L, A97L, A188F, K93W, T90I, A174I, K93Y, A111L, A97I, N130A, G48I, E278W, R221Y, G135L, T I12V, F197V, T144F, K93S, G194V, G87A, A188V, T144C, K46N, P115M, A175I, N130T, S51A, G194C, G48L, G104V. A174V, G158V, T90V, R221N, A202L, A39L, A134F, D137Y, G158I, A174L, P155V, A171V, S51T, F197I, T112C, D137W, G135V, D137G, P115G, T250C, S208L, A100I, T250L, T144M, T108C, P115F, G69L, T112F, G181F, A100F, S208I, G223V, T250V, N249M, A111V, N249Y, A111F, A188M, A111I, P209Y, A175V, P1551, N130G, S120V, A111C, A100C, A97F, G194M, G69I, E278Q, S2080, G181C, G87F, G48V, A97C, I205L, G194I, D137T, S51G, F197C, G223L, N249A, S51Y, P115S, A2021, P155C, S26A, G83M, S120C, D137S, E278N, G83F, G158C, P59Y, G87C, G87M, N130S, P115I, A202F, A100M, G83C, S212M, A39V, A188I, F197L, G181M, A134C, P115T, A174F, K93P, P209W, P209G, P59A, A39C, G104I, T250G, S208F, R221Q, G181A, 0104C, T90C, K46Q, P209T, K46W, A171I, G158A, A134M, G48C, G194A, T90A, T90M, N130Y, N249F, A172L, A152V, A152I, P59W, R221P, G158F, T112M, G135M, G181V, G104L, S208T, N249W, A97M, G135F, K93Q, G42L, A111M, G104A, E278P, T90L, R221D, A175C, A188C, A291L, S212A, F123L, T250I, A174C, E164Q, P155L, A174M, G158M, A172V, E164N, A168L, T90F, A39F, G42V, I205F, A39M, N249G, A175F, G135C, A171F, S212F, K46D, A100Y, G421, P59G, G69A, E164P, G158L, G42C, A171C, T250M, T108L, K93N, A171M, G223I, A175M, T108M, A1721, S51P, A291I, A111Y, G20V, S120I, P59T, A58I, G223C, S208M, G48M, K46H, A62L, A431, T250A, N130W, G194Y, R221H, D137P, N249S, G104F, A100V, F123I, A172C, T112I, G69F, P115Y, P59S, A100W, G104M, G69C, R221W, K93H, A204L, G20L, G223F, A168I, D28K, E275K, K77R, D17R, H76D, H75D, H76E, K77D, H75E, P294D, S293D, S293E, D286E, P294E, S292E, D285E, H76R, K77E, K289R, P295D, D285K, R179E, H75R, P295E, P294R, P294K, D286K, D285R, S293R, S293K, R180D, R180K, H183E, R179K, K289E, R180E, A98K, R179D, A99K, S292D, D286R, K290D, H75K, A99E, N256E, K290E, K290R, R239D, A199K, A200K, K255E, H183D, A99R, H235E, A98R, D102K, and A200R,

The isoelectric point of a protein is the pH where the molecule carries, on average, no net charge, i.e. where positive and negative charges are in equilibrium. Above this pH value charge will be more negative, below this it will be more positive. Importantly, the isolelectric point is where proteins are typically least soluble. The pI for DsA1, DsA2 and PITP are approximately within the range of 9.5 to 11, i.e. fairly basic, but contain domains which are more acidic or at least neutral (e.g. the ENFD of PITP or regions in the C-terminus of PITP (especially the region from S397 to T430)). In order to provide pH stability, it is therefore preferred to exchange aspartic or glutamic acid and histidine residues with basic residues, such as arginine and lysine in these neutral/acidic regions to increase solubility of the whole polypeptide. Preferred amino acid exchanges for establishing improved pH stability are therefore (for each position starting from the most preferred exchange) D152F, D152L, D152I, D152V, D152M, D152W, D152Y, D152S, D152A, D152T, D152G, D152N, D152Q, D152P, D152H, D152K, D152R, D152C, D155Q, D155L, D155F, D155I, D155M, D155W, D155V, D155Y, D155A, D155N, D155T, D155S, D155P, D155G, D155H, D155R, D155K, D155C, D156L, D156F, D156I, D156W, D156V, D156A, D156Y, D156M, D156G, D156S, D156T, D156Q, D156N, D156P, D156H, D156K, D156R, D156C, H146R, H146K, H153R, H153K.

Another group of preferred amino acid exchanges in PITP derivates concerns the ENFD and the HbD. Both domains are capable of binding/coordinating porphyrin molecules and can therefore also be referred to as “porphyrin binding domains”. Various porphyrins are produced by P. acnes, also depending on environmental conditions, such as the amount of oxygen, pH, etc., the genetic type (strain differences), growth medium, cell activity/growth stage (e.g. planktonic vs biofilm, early log phase vs stationary phase—age of culture), enzyme activity, such as UP decarboxylase, CP oxidase, etc. (see e.g. Miah. Biotechnol. 1 (2002), 21-27; Shu et al., Curr. Med. Chem. 20 (2013), 562-568). Under anaerobic conditions, protoprophyrin IX is usually dominant. On the other hand, under aerobic conditions, coproporphyrin III is usually predominantly the final product of the P. acnes porphyrin metabolic pathway. The structural difference between heme and P. acnes porphyrins (protoprophyrin IX or coproporphyrin I1) is that heme has the addition of metallation into the middle ring structure of protoporphyrin IX catalyzed by enzyme ferrochelatase. In P. acnes, the heme within the porphyrin ring gets bonded by a metal atom, by the action of ferrochelatase, the terminal enzyme of the heme biosynthetic pathway in all cells. It catalyzes the insertion of ferrous iron into protoporphyrin IX, yielding heme. Therefore, protoporphyrin IX is produced by P. acnes only when there is enough iron in the external environment which means that P. acnes competes for iron with human immune cells which also need it for their metabolism, as well as with other bacteria which also live on the skin (e.g. S. aureus, S. epdiermidis). Colonizing the human skin requires the bacterium to tolerate oxygen and be able to protect itself from ROS (Allhorn et al., Sci. Rep. 6 (2016): 36412; Dutra et al., Front. Pharmacol. 5 (2014), Art. 115). Several enzymes involved in redox reactions, that are able to counteract reactive oxygen species release and damage induced by skin exposure to UV light or phagocytes, rely on heme as a co-factor for their activity. P. acnes expresses several of such enzymes.

In the vaccine products according to the present invention it may be preferred to provide polypeptides which lack porphyrin binding abilities (especially heme binding abilities) because presence of such functional binding domains may have negative impact on recombinant production of these polypeptides in hosts which produce porphyrins that could interfere with the physiological binding partner or because presence of spectroscopically active compounds such as porphyrine ligands (in particular heme ligands) may be less desired in a vaccine product. Accordingly, the following amino acid exchanges in the ENFD and the HbD domains of PITP which decrease or prevent porphyrin (especially heme) binding are preferred (for each position starting with the most preferred exchange): ENFD: exchange at positions Y63, H146, H153, F74, L141, F81, P72, K144, T143, Y75, W98, D156, and R158; HbD: exchange at positions S250, F251, Y254, I259, H306, I309, L310, M312, S334, S335, M337, A376, F377, A378, F380, and Y381.

At these positions, exchange is preferably by alanine (ENFD: Y63A, H146A, H153A, F74A, L141A, F81A, P72A, K144A, T143A, Y75A, W98A, D156A, and R158A, or by arginine, aspartic acid, glutamic acid, lysine, etc. (HbD, for each position starting with the most preferred exchange: S250W, S250F, S250D, S250E, S250K, S250R, F251A, Y254A, I259W, I259F, I259D, I259E, I259R, I259K, H306A, I309W, I309F, I309E, I309D, I309R, I309K, L310W, L310F, L310D, L310E, L310R, L310K, M312W, M312F, M312D, M312E, M312R, M312K, S334W, S334F, S334D, S334E, S334R, S334K, S335W, S335F, S335D, S335E, S335K, S335R, M337W, M337F, M337D, M337E, M337K, M337R, A376W, A376F, A376E, A376D, A376R, A376K, F377, A378W, A378F, A378D, A378E, A378K, A378R, F380A, Y381A, D348I, D348L, D348M, D348F, D348V, D348Y, D348A, D348W, D348T, D348G, D348P, D348H, D348S, D348R, D348K, D348E, D348C, H306R, H306D, H306E, H306K, H307D, H307R, H307E, H307K, H327R, H327K, H327E, H327D, H302E, H302K, H302R, and H302D.

Another strategy to provide pharmaceutical preparations comprising PITP polypeptides and/or a fragment and/or a derivative thereof which lack or have reduced amounts of porphyrins and other substances which are in principle able to bind to PITP is to recombinantly express the polypeptides in the absence of porphyrins and other substances which are in principle able to bind to PITP and/or by recombinant host cells which lack such porphyrins and porphyrin-like substances. Finally, it is also possible to extract porphyrins and other substances which are in principle able to bind to PITP after expression before finalizing the pharmaceutical formulation. If a given amount of porphyrin should be present in the final pharmaceutical preparation, it is also possible to generate the initial expression product without the porphyrin and add (a predetermined amount of) porphyrin later in the course of pharmaceutical formulation.

Advantages of a formulation which is free of porphyrins and other substances which are in principle able to bind to PITP (or has a reduced content of such substances) are that such a pure(r) formulation or expression product allows to generate a chemically defined product. Adding the porphyrin alternatively allows the provision of a product with a predefined protein/porphyrin content, with a specific porphyrins or mix of porphyrins. i.e. it allows to fully control product composition, not only to monitor it. Addition of porphyrins, especially hemes may be performed by adding porphyrins and porphyrin-like substances (binding in principle to PITP) to the culture, to the lysis buffer, and/or to the purified protein.

When the polypeptide is loaded with porphyrins after production it should not be less stable than e.g. an E. coli version, only more defined in composition.

In case there are functional porphyrins (or porphyrin-like ligands) this would also be the most natural choice as otherwise there are always some E. coli porphyrins added as well. Accordingly, really both porphyrin free and high-porphyrin embodiments (and variations in types of porphyrins, especially hemes) are possible and enabled by the present invention, and each may be advantageous, depending on precise application and dictated tolerances and requirements.

An example for a suitable host cell for expression without porphyrins is Lactobacillus lactis. L. lactis. Is probably the most relevant biotechnological host cell (used specifically for protein production) which is naturally unable to synthesize porphyrins/heme (besides a few other lactobacilli which are relevant e.g. in food industry). Technically there are many other species lacking heme synthesis capacity, mostly a choice of species favoring an anoxic lifestyle or able to take up heme from the environment, such as other lactic acid bacteria, certain round worms and helminths (Rao et al., PNAS 102 (2005), 4270-4275), etc. (basically the entire group of bacteria the Lactobacilli belong to, are unable of heme synthesis).

L. lactis cannot synthesize heme, but can utilize it, i.e. it must not be provided in the medium, and in consequence somewhat less efficient anoxic fermentation has to be used to avoid oxygenic stress. It is preferred to add glucose in routine production to further limit tendency for oxygenic growth.

If porphyrins and porphyrin-like substances (binding in principle to PITP) are used to stabilize the pharmaceutical formulations according to the present invention, the porphyrins and porphyrin-like substances may, as already stated above, be added to the culture, to the lysis buffer, and/or to the purified protein. Moreover, a production strain may be used that is optimized for the heme/porphyrin production.

Another interesting feature of L. lactis is its (partial) ability to glycosylate surface proteins to a certain degree (Theodorou et al., JBC 295 (2020), 5519-5532). Accordingly, expressing the polypeptides according to the present invention by a host cell which is able to glycosylate the recombinant expression product may be advantageous as well. E. coli rarely glycosylates, however P. acnes has at least one strongly glycosylated protein (modified with GalNAc). In some embodiments of the present invention, glycosylation can improve both solubility and antigenicity.

According to the present invention, L. lactis is therefore another preferred prokaryotic host cell for expressing the recombinant polypeptides according to the present invention. Further preferred amino acid exchanges in PITP to arrive at polypeptides with improved solubility (especially upon increased pH) are exchanges of histidine residues outside the ENFD and HdD domains. Preferred examples of such amino acid exchanges are (for each position starting with the most preferred exchange) H215K, H215R, H215E, H215D, H223K, H223R, H223E, H223D, H403K, H403R, H403E, and H403D.

In summary, amino acid exchanges may be provided along the following principles. The exchanges should affect the antigenic surface of a given target epitope as little as possible. B buried acidic residues should be eliminated to increase pH stability. Surface accessible lysines should be exchanged against residues charged at higher pH values to optimize solvation (better solvation means protein should be better soluble at higher concentration and lower salt concentrations). Exchanges of surface residues can increase the iso-electric point of the entire protein, but specifically specific domains and regions, foremost the ENFD. The rationale is that the isolelectric point of a protein is the pH where not solvation is typically minimal. Exchanges of surface residues may add charge and increase solubility, however, as stated above, care must be taken that specific target epitopes are not or not significantly altered. Exchanges of the heme binding residues of HbD and ENFD can affect heme binding, but naturally also solvation and stability. Further amino acid exchanges may be confirmed both, experimentally and by computer modelling (e.g. with public methods, such as DeepDDG (Cao et al., J. Chem. Inf. Model. 59 (2019), 1508-1514) and PremPS (Chen et al., Comput. Biol. 16 (2020), e1008543)).

The deletions, insertions, and substitutions of the amino acid sequence of a variant are not expected to produce significant changes in the characteristics of the protein. However, when it is difficult to predict the exact effect of the substitution, deletion, or insertion in advance of doing so, one skilled in the art will appreciate that the effect will be evaluated by functional assays as exemplified herein.

This is why these exchanges are also appropriate for the epitopes of the present invention. Accordingly, also the epitopes R32-141, Q38-K51, R32-K51, T43-K51, Q38-K51, R87-K90+T43-K51, R87-K90+T117-I132 and R87-K90+S234-G250, R87-K90+L246-A260, R87-K90+A256-E270, R87-K90+R266-T277, T117-I132, T117-A127, V128-I132, A144-N157, H146-A160, A148-N157, A156-A170, K166-L180, A176-T190, P186-A198, N181-E191, I216-F224, I216-D225, A226-A24G, S234-G250, I251-I263, I264-P271, P236-G250, L246-A260, A256-E27G, S234-G250, I251-I263, I251-L267, A268-L280, R266-T277, T285-R286+I216-F224, T285-R286+I264-P271, T285-R286+V289-K296, T285-R286+V289-K296, T285-R286+A144-N157, A310-D313+T285-R286, T285-D290, T285-D290+V291-T300, T285-D290+A301-E307, V291-T300, A301-E307, T285-T300, A301-E307, R286-D290+V291-T300, R286-D290+A301-E307, R286-T300, V289-K296, A310-D313+I216-F224, A310-D313+I264-P271, A310-D313+T285-R286, A310-D313+R286-D290, A310-D313+V289-K296, A310-D313+V289-K296, A310-D313+T285-T300, A310-D313+A144-N157, A310-D313+T285-R286, A310-D313+T285-D290, A310-D313+T293-E307, T285-D290, V291-T300, T293-E307, A301-E307 of DsA1; L152-Q166, G190-P230, I199-D208, A218-I237, P230-Q244, I231-A270, H254-A270, A271-S279, A271-R310, L311-T321, L311-K323, V333-Q347, A218-P230, I231-I237, H254-H262, Q256-H262, E261-D269, D269-S279, K313-K323, of DsA2; D79-T90, E73-D85, R43-150, P68-Y75, P86-E92, I39-G45, Y84-D89, F81-D89, D79-T90, T37-E44, E73-W98, E73-F81, D89-T90, P72-F81, A129-F138, D120-Q134, F111-D120, F132-G147, D152-E165, R115-F123, D120-K128, P131-F138, N181-E19I, T143-T159, P116-T124, P131-D137, P131-D137, T175-C231, Q198-K203, P179-K185, G200-Q210, K174-A188, K174-K185, P201-Q209, P183-P201, P183-K191, K185-P195, R164-S180, E165-S180, K185-S190, V193-N202, V193-G200, K203-P208, R216-T225, R216-R224, P173-K191, K197-K203, P168-T175, K185-K203, R164-K174, T175-V193, S250-N261, D287-S300, K340-V347, D338-F352, D338-D348, S285-P288+G305-L314, S285-P288+H306-L314, S285-P288+T342-T351, S285-P288+D338-D348. D287-S300, T342-T351, D338-D348, H306-L314, G305-L314, G364-K375, R382-E399, V367-G373, A383-L390, T342-T351, M387-T395, E385-T392, V401-V410, N404-A409, G416-L427, L396-V410, T406-1415, D417-G424, V407-D418, V407-V414, K421-V429, S419-T430, D408-I415, T406-V414, of PITP; which contain one or more, preferably one single exchange, as disclosed herein are provided by the present invention.

The term “fragment” refers to a portion of an amino acid sequence. Fragments of a target antigen specifically will comprise or consist of at least 6, preferably at least 8, especially at least 10 contiguous amino acids, or up to the total number of amino acids present in a full-length target antigen as described herein. Preferred fragments of DsA1, DsA2 and PITP at least comprise an antigenic epitope (i.e. an epitope which is immunogenic and accessible to antibody binding on the surface of P. acnes) or have a length of at least 20 amino acids, preferably at least 30 amino acids, especially at least 50 amino acids of a naturally occurring DsA1, DsA2, or PITP protein. Based on the definitions herein, fragments of derivatives are, of course, derivatives and are fragments of derivatives which contain one or more amino acid exchanges, insertions or deletions disclosed herein.

A fragment of a target antigen can be prepared by isolating a portion of a target antigen and assessing the protective activity of a fragment, or by synthetizing a peptide corresponding to the immunologically relevant epitope.

“Percent (%) amino acid sequence identity” as used herein with respect to protein sequences shall mean the percentage of amino acid residues in a candidate sequence that are identical with the amino acid residues in the protein sequence, after aligning the sequences and introducing gaps, if necessary, to achieve the maximum percent sequence identity, and not considering any conservative substitutions as part of the sequence identity. When performing alignment on two different proteins a pairwise identity is determined using an algorithm searching for and aligning similar regions. As used herein the term “sequence identity” shall also encompass such “sequence similarity”.

The polypeptides (e.g. proteins, antigens, epitopes, fragments, derivatives, etc.) as described herein may be artificial or non-naturally occurring. The term “naturally occurring” refers to proteins which are present in naturally occurring P. acnes isolates and which are not amended by recombinant DNA/RNA technology. A naturally occurring DsA1, DsA2 or PITP protein is a protein which is isolated from natural sources, e.g. from the skin of human individuals. The term “non-naturally occurring” or “artificial” with respect to polypeptides (e.g. proteins, antigens, epitopes, fragments, derivatives, etc.) refers to synthetic polypeptides which are not isolated from natural sources and have an amino acid sequence which is different from naturally occurring P. acnes proteins. For example, the non-naturally occurring polypeptides are produced by recombinant DNA or RNA technology and may contain fragments of naturally occurring P. acnes protein. “Non-naturally occurring” or “artificial” compounds have an amino acid sequence, a structure and/or function not found in nature.

The term “recombinant” as used herein simply refers to any protein, polypeptide, or cell expressing a gene of interest that is produced by genetic engineering methods. The term “recombinant” as used with respect to a protein or polypeptide, means a polypeptide produced by expression of a recombinant polynucleotide. “Recombinant,” as used herein, further describes a nucleic acid molecule, which, by virtue of its origin or manipulation, is not associated with all or a portion of the polynucleotide with which it is associated in nature. The term “recombinant” as used with respect to a host cell means a host cell into which a recombinant polynucleotide has been introduced.

The term “substantially the same” with regard to function, effect or activity of a variant or derivative as used herein refers to the activity being at least 20%, at least 50%, at least 75%, at least 90%, e.g. at least 100%, or at least 125%, or at least 150%, or at least 175%, or e.g. up to 200% of the activity as determined for the comparable.

The term “specific binding” or “specificity of binding” as used herein shall refer to a binding reaction which is determinative of the cognate ligand of interest in a heterogeneous population of molecules. Thus, under designated conditions, e.g. immunoassay conditions, the antibody that specifically binds to its particular target antigen does not bind in a significant amount to other molecules present in a sample, unless there is a larger than 50% amino acid sequence similarity, which could result in certain common epitope sequences or structures. The term shall specifically apply to an antibody that specifically binds to pathogens of the same, i.e. specific species. As further described herein, in certain embodiments it is desirable to employ those antibodies that are species-specific, however cross-binding specifically binding more than one target proteins of a species, and/or being cross-reactive/cross-type-reactive specifically binding different subspecies and/or serotypes of the same species.

Antibodies with a specific binding site are typically not cross-binding with other targets. It is therefore surprising that cross-binding or cross-reactivity, especially cross-type-reactivity, of antibodies was found as further described herein, specifically for DsA1, DsA2 and PITP which show induction of antibodies with significant cross-type binding properties.

Antibodies are said to be cross-binding if binding to the same epitope of different antigens.

Specific binding an antigen or epitope by an antibody means that binding is selective in terms of target identity, high, medium or low binding affinity or avidity, as selected. Selective binding is usually achieved if the binding constant or binding dynamics is at least 10-fold different, preferably the difference is at least 100-fold, and more preferred a least 1,000-fold. The preferred method for determining semi-quantitative and qualitative affinity characterization according to the present invention is the surface plasmon resonance technique (referred to as “SPR” or “Biacore” analysis). With this method, biomolecular interactions, including protein-protein interactions, small molecule/fragment-protein interactions, etc. are measured not only with respect to binding affinities, but also with respect to kinetic rate constants and thermodynamics. The technology is based on an optical phenomenon that enables detection of unlabeled interact ants in real time, i.e. SPR. SPR-based biosensors are used in determination of active concentration as well as characterization of molecular interactions in terms of both affinity and chemical kinetics (Myszka et al., Biophys. J. 75 (1998), 583-594). A preferred instrument for performing SPR according to the present invention is Biacore T200. SPR allows the label free detection of protein-protein interaction in real-time and is therefore most suitable for the binding characterization of an antibody to its antigen. Briefly, SPR occurs when polarized light strikes an electrically conducting surface at the interface between two media. This generates electron charge density waves called plasmons, reducing the intensity of reflected light at a specific angle known as the resonance angle, in proportion to the mass on a sensor surface. In Biacore assays, target molecules, in this case an antigen, is immobilized on a prepared chip surface and a sample containing a potential interacting partner, in this case the serum antibody in solution is injected over the surface through a series of flow cells. During the course of the interaction, polarized light is directed toward the sensor surface and the angle of minimum intensity reflected light is detected. This angle changes as molecules bind and dissociate and the interaction profile is thus recorded in real time in a sensorgram. During sample injection, a positive response can be viewed in the sensorgram, as analyte (the interacting partner in solution (serum/antibody) binds to the ligand (the interaction partner that is attached to the sensor chip (antigen). The response decreases during dissociation. After an analysis cycle is completed, regeneration solution is passed over the sensor chip, removing bound analyte, preparing for the next analysis cycle. For the semi-quantitative and qualitative (affinity) characterization of vaccine induced ABs, a Streptavidin-chip was used to immobilize the three Avi-tagged proteins on three individual flow cells (Fc).

“Isolated” in the context of the present invention with respect to polypeptides, antigens, and epitopes means that the material is removed from its original environment. An isolated antigen or epitope can specifically be separated from other antigens or epitopes that are naturally associated, such as to create an artificial immunogen that includes the isolated antigen and/or epitope. “Isolated” does not necessarily mean the exclusion of artificial or synthetic mixtures with other compounds or materials, or the presence of impurities that do not interfere with the fundamental activity, and that may be present, for example, due to incomplete purification. The polypeptides, antigens, and epitopes disclosed and claimed herein (and specifically defined in the claims and embodiments) are generally regarded as being disclosed and claimed in their isolated form.

An isolated antigen may represent a molecule produced directly by biological or synthetic means and separated from other components present during its production. An isolated or purified antigen as described herein may further refer to an antigen comprising a reduced amount of other material derived from cell culture media or sera, or is preferably substantially free of such other material, and which specifically comprises a reduced amount of antigens that induce antibodies which were found to be non-protective in a functional assay for determining the antibacterial effect.

The term “substantially pure” or “purified” as used herein shall refer to a preparation comprising at least 50% (w/w protein or polypeptide content), preferably at least 60%, 70%, 80%, 90% or 95% of a compound, such as an antigen or an antibody. Purity is measured by methods appropriate for the compound (e.g. chromatographic methods, polyacrylamide gel electrophoresis, HPLC analysis, and the like).

Isolation and purification methods are e.g. utilizing difference in solubility, such as salting out and solvent precipitation, methods utilizing difference in molecular weight, such as ultrafiltration and gel electrophoresis, methods utilizing difference in electric charge, such as ion-exchange chromatography, methods utilizing specific affinity, such as affinity chromatography, methods utilizing difference in hydrophobicity, such as reverse phase high performance liquid chromatography, and methods utilizing difference in isoelectric point, such as isoelectric focusing may be used. The isolated antigens can be identified by conventional methods such as Western blot, HPLC, activity assay, flow cytometry or ELISA.

Preferably, the vaccine as described herein is a “subunit vaccine” of P. acnes, which is herein understood as a vaccine which does not comprise the whole P. acnes bacterium, or compositions comprising inactivated bacteria. Such a vaccine comprises distinct chemically defined components (e.g., an immunogen) and is substantially free of intact bacterial cells or bacterial particles, or inactivated cells or the lysate of such cells or particles. A subunit vaccine can be prepared from at least partially purified, or substantially purified, polypeptides from the pathogen. Methods of obtaining an antigen or antigens in the subunit vaccine or subunit composition include standard purification techniques, recombinant production, or chemical synthesis. A P. acnes “subunit vaccine” thus refers to a vaccine or composition consisting of a defined (or artificial) antigenic component or components of P. acnes.

The term “P. acnes” as used herein shall refer to the bacterial cell in the isolated form or grown in a cell culture, or strains of P. acnes obtained from human subjects. Exemplary P. acnes strains include NCTC737, KPA171202, which can be obtained from National Collection of Type Cultures (Colindale, UK), SK137, HL005PA1, HL005PA4, HL013PA1, HL030PA1, HL043PA1, HL053PA1, HL053PA2, HL050PA1, HL050PA2, HL060PA1, HL110PA4 (BEI, Biodefense and Emerging Infections Research Resources Repository. Manassas, VA) and IAI 008, IAI031, IAI034, IAI035, IAI038, IAI040, IAI042, IAI045, IAI041 (Charite Berlin, Pro-Implant foundation). The strains can be obtained from Global Bioresource Center ATCC (Manassas, USA). Leibnitz Institute DSMZ (Braunschweig, Germany). BEI, Biodefense and Emerging Infections Research Resources Repository (Manassas, VA), and other commercial sources; the strains that appear in the public sequence databases, such as NCBI genome database; the strains studied and referred to in scientific publications (McDowell et al. 2012; Tomida et al. 2013) and the strains isolated from the skin of human subjects in the approved clinical studies. The term specifically includes the strains which have the pathogenic potential and are isolated from different P. acnes-associated disorders.

P. acnes phylotypes are understood as follows:

Two distinct phenotypes of P. acnes (Types I and II), can be distinguished by serological agglutination tests and cell-wall sugar analysis: the cell walls of the Type I strains contain galactose, glucose and mannose, whereas Type II strains contain only glucose and mannose. Additional studies have shown that these biovars display differences in the fermentation of sugar and sugar alcohols, as well as their susceptibility to bacteriophage infection. Sequence analysis of the P. acnes recA gene has revealed that Types I and II correspond to phylogenetically distinct clusters or lineages. These two clusters are, however, almost identical based on 16S rRNA sequencing. Analysis of the recA gene has also identified a subcluster of strains within P. acnes Type I that have been designated Type IB.

More detailed phylogenetic analysis based on MLSTs sequence data has, however, revealed that all Type IA isolates can be further partitioned into one of two distinct, statistically significant clades, which they have designated Types IA1 and IA2. These two distinct divisions are further supported at the phylogenomic level.

Specifically, Type IA1 is characterized by the high abundance of this type isolated from lesions of acne vulgaris patients. Specifically. Type IA2 is characterized by containing fewer non-core genomic regions when compared with Type IA1 strains, which may result from a lack of rearrangement hot spot family proteins.

Type III strains are characterized by atypical cellular morphology. Unlike the classical coryneform morphology seen with P. acnes Types I and II (i.e. clubs, ‘tadpole’ forms and short bifid forms), Type III isolates consist of individual cells of variable length and long slender filaments that form very large tangled aggregates. Type III isolates are positive for catalase activity, negative for sorbitol and erythritol fermentation, but could ferment ribose, glucose and glycerol. Additionally, Type III strains are most frequently associated with the infections affecting spine invertebral disc material, implant-associated infections and other types of P. acnes infections, and they have been also suggested to be specifically associated with progressive macular hypomelanosis (PMH) (Barnard, 2016, Dagnelie, 2018).

The typing schemes used by different authors are well described in the literature (Lomholt and Kilian 2010; McDowell et al. 2012; Fitz-Gibbon et al. 2013; Scholz et al. 2014; Barnard et al. 2015; O'Neill and Gallo 2018; McLaughlin et al. 2019).

P. acnes strains can be divided into groups using genomic sequencing of 16S rDNA sequence called a ribotype (RT). This system allowed to compare the P. acnes strain populations in individuals based on the 16S rDNA sequences. The top 10 major ribotypes were highly abundant while also a significant number of rare ribotypes were identified. All of the top 10 most abundant ribotypes differ from RT1 by only one or two nucleotide changes in the 16S rDNA sequence. According to the analysis of the top 10 ribotypes both disease-specific and health-specific associations could be identified (Fitz-Gibbon et al. 2013; Tomida et al. 2013; McLaughlin et al. 2019). The three most abundant ribotypes (RT1, RT2, and RT3) were fairly evenly distributed among acne and normal individuals. However, ribotypes 4, 5, 7, 8, 9, and 10 were found predominantly in acne patients, while RT6 was strongly associated with normal skin. A phylogenetic tree based on unique single-nucleotide polymorphism positions in the core genome obtained from these 71 P. acnes genomes suggested that the 16S rDNA ribotypes to a large extent represent the relationship of the lineages, and that the 16S rDNA sequence is a useful molecular marker to distinguish major P. acnes lineages (Fitz-Gibbon et al. 2013; Tomida et al. 2013).

A “P. acnes indication”, a “P. acnes-associated infection”, a “P. acnes-associated disease” or “P. acnes-associated infection and pathological conditions associated with any of Type I, II, or III P. acnes” is herein understood as a disease or disorder associated with P. acnes increased pathogenic activity or proliferation within a colonizing site (e.g. a skin hair follicle) or spread to the new sites not colonized by P. acnes (infection), in particular a disease or disorder that involves e.g. is caused, exacerbated, or characterized by the presence of P. acnes bacteria residing and/or replicating in the body and/or cells of a subject. Thus, these terms cover any disease, disorder, pathology, symptom, clinical condition or syndrome in which bacteria of the species P. acnes act as etiological agent or in which infection with one or more strains of P. acnes is implicated, detected or involved. These terms therefore include acne vulgaris, including acne fulminans, acne conglobata and acne inversa, keratitis, synovitis acne pustulosis hyperostosis osteitis (SAPHO) syndrome, endocarditis, medical implant biofilm infection, including prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, and shunt-associated central nervous system infections, sarcoidosis, endophthalmitis, osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, and pulmonary angitis, acne inversa, progressive macular hypomelanosis, acne conglobata, artherosclerosis, prostethic cancer and a medical implant biofilm infection by P. acnes (see e.g. Bruggemann, Skin: Acne and P. acnes Genomics, in Timmis (ed.), “Handbook of Hydrocarbon and Lipid Microbiology”, DOI 10.1007/978-3-540-77587-4_244; Springer-Verlag Berlin Heidelberg, 2010, pages 3215 to 3225; Portillo et al., BioMed Res. Int. 2013, ID 804391, Achermann et al., Clin. Microbiol. Rev. 27 (2014), 419-440; Capoor et al., Eur. Spine J. 28 (2019), 2952-2971; McDowell et al., BioMed Res. Int. 2015, ID 493564).

Accordingly, the present invention also relates to a method of treatment or prevention of P. acnes-associated infections in a human patient suffering from P. acnes-associated infections and pathological conditions associated with any of Type I, II, or II P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes comprising administration of an effective amount of a DsA1 and/or DsA2 and/or PITP, and/or a fragment and/or a derivative of DsA1 and/or DsA2 and/or PITP according to any one of claims 5 to 27 or mixtures thereof in an effective amount to a patient in need thereof.

According to a preferred embodiment, administration is performed by intradermal, subcutaneous (s.c.), parenteral, intramuscular (i.m.), mucosal, transcutaneous or topical administration, preferably by intradermal, or intramuscular administration, especially by a syringe or by microneedeling devices.

The present invention also refers to the use of DsA1 and/or DsA2 and/or PITP, and/or a fragment and/or a derivative of DsA1 and/or DsA2 and/or PITP according to the present invention or mixtures thereof for the manufacture of a medicament for treatment or prevention of P. acnes-associated infections in a human patient suffering from P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes.

Acne vulgaris (common acne) is the formation of comedones, papules, pustules, nodules, and/or cysts as a result of obstruction and/or inflammation of pilosebaceous units (hair follicles and their accompanying sebaceous gland). It most often affects adolescents. Acne can be inflammatory or non-inflammatory. Acne vulgaris typically affects the areas of skin with the densest population of sebaceous follicles (eg, face, upper chest, back). Local symptoms of acne vulgaris may include pain, tenderness, or erythema. Acne conglobata is a rare but severe form of acne. It usually presents with deep burrowing abscesses that interconnect with each other. Scar formation and disfigurement of the body are common with this type of acne. The comedones often occur in groups of three and the cysts often contain purulent, foul smelling material that is discharged on the skin surface. This severe form of acne frequently heals with disfiguring scars. Severe acne with associated systemic signs and symptoms, such as fever, is referred to as acne fulminans.

Biofilm formation is a well-known process where a microorganism attaches to a suitable tissue or material surface and produces extracellular polymers leading to adherence and matrix formation. Microbial biofilm cells can adhere to the exopolysaccharide matrix present on the surface of medical devices and adversely affect the function of the device. Bacteria that attach to surfaces aggregate in a hydrated polymeric matrix of their own synthesis to form biofilms. Formation of these sessile communities and their inherent resistance to antimicrobial agents are at the root of many persistent and chronic bacterial infections.

Biofilm-associated infections can be broadly divided into two types: infections associated with indwelling medical devices and native biofilm infections of host tissues. For the former type, bloodstream or urinary tract infections can be caused by infectious biofilms originally formed on the surfaces of indwelling medical devices, such as central venous catheters, mechanical heart valves, urinary catheters, joint prostheses, peritoneal dialysis catheters, cardiac pacemakers, cerebrospinal fluid shunts, endotracheal tubes, contact lenses, intrauterine devices and dental unit waterlines. In these cases, pathogens may originate from the epithelial flora of patients, healthcare personnel or other sources in the environment, to form infectious biofilms on the surfaces of indwelling medical devices, and subsequently gain access to human organs or tissues via indwelling medical devices inserted into the human body. Native biofilm-associated infections are often chronic, opportunistic infections in otherwise sterile locations of the human body, and mainly include chronic lung infections of cystic fibrosis patients, chronic otitis media, native valve infectious endocarditis, chronic osteomyelitis, chronic rhinosinusitis, chronic prostatitis, recurrent urinary tract infection, chronic wounds, dental caries and periodontitis. Biofilm-associated infections can be caused by a single microbial species or by a mixture of species, with interactions between multiple species increasing their persistence.

P. acnes biofilm is understood as clusters of bacteria including P. acnes that are attached to a surface, such as skin or artificial surfaces (e.g. prosthetics and surgical implants, contact lenses, catheters and other medical devices), and are embedded in a slime layer on that surface. They are produced by the bacteria and serve as a natural self-protection mechanism. Bacterial cells growing within the P. acnes biofilm exhibit increased resistance to antimicrobial agents. The formation of a P. acnes biofilm has been proposed to be the reason why antimicrobial agents used in acne therapy fall short of their therapeutic goal, fueling research to develop targeted therapies to stave off the impact of biofilms in acne vulgaris.

Biofilm formation by P. acnes is considered to be one of the key factors underlying the pathogenesis of acne and other P. acnes-associated infections. P. acnes biofilm leads to increased virulence evidenced by a higher activity of certain enzymes (e.g. lipase) and a dramatically elevated resistance of P. acnes to antimicrobial agents (Burkhart and Burkhart 2007; Coenye et al. 2007). It was shown via microscopic visualization that macrocolonies in sebaceous follicles of the skin were more frequently found in acne patients than healthy individuals and that the ability to form biofilms was a characteristic of invasive isolates (Holmberg et al. 2009; Jahns et al. 2012).

As used herein, the term “therapy” specifically refers to immunotherapy, which is herein understood as a treatment, for example, a therapeutic or prophylactic treatment, of a disease or disorder intended to and/or producing an immune response, e.g., an active or passive immune response.

The vaccine as described herein may suitably be used for immunotherapy of a subject in need thereof, wherein a therapeutically effective amount of antigen material is administered to said subject.

Herein, the term “subject” is understood to refer to a human being, particularly any of a child (up to 10 years of age), adolescent (e.g. 10-18 years of age), or adult (e.g. above 18 years of age). A subject in need of prophylaxis or treatment of an infectious disease condition caused by a pathogen, in particular a microbial pathogen, including e.g. bacteria, may be specifically a patient suffering from disease, including early or late stage disease, or else a subject at risk of disease.

A subject in need of prophylaxis or treatment of a P. acnes indication may be specifically a patient suffering from disease, including early or late stage disease, or else a subject predisposed or being at risk of such disease, e.g. by the potential exposure to the pathogen via novel route or contact, or being exposed to a higher pathogenic load. The terms “susceptible to,” and “at risk of,” as used herein, are used interchangeably to refer to individuals having little resistance to a certain condition or disease, including being genetically predisposed, having a family history of, and/or having symptoms of the condition or disease. In some embodiments, a subject is suffering from a disease associated with P. acnes infection that has proven refractory to treatment with other conventional therapy.

The term “treat” or “treating”, as used herein, refers to reversing, alleviating, inhibiting the progress of, or preventing a disorder, condition or disease to which such term applies, or to preventing one or more symptoms of such disorder, condition or disease. Preferably, the method for treating, preventing, or delaying a disease condition in a subject as described herein, is by interfering with the pathogenesis of P. acnes in a P. acnes indication.

As used herein “treating acne” means preventing, retarding and/or arresting the process of acne formation in mammalian skin.

“Therapeutic or prophylactic treatment” as used herein, refers to treatment with an antigen or vaccine as described herein that would lead to an immune response in a subject receiving the antigen or vaccine which is adequate to prevent or ameliorate signs or symptoms of disease, including adverse health effects or complications thereof, caused by infection with P. acnes. Humoral immunity or cell-mediated immunity, or both humoral and cell-mediated immunity, can be induced. The immunogenic response of an individual to a vaccine can be evaluated indirectly through measurement of antibody titers, lymphocyte proliferation assays, or directly through monitoring signs and symptoms after challenge with the wild type strain. The protective immunity conferred by such vaccine can be evaluated by measuring reduction of challenge organism shed, and/or reduction in clinical signs, such as mortality, morbidity, temperature, and overall physical condition, health, and performance of the subject. In the context of the present invention, therapeutic or prophylactic treatment of a P. acnes associated disease can be monitored by the appearance of the skin (e.g., presence of comedones papules, pustules, and nodules), symptoms of red eye, pain, sensitivity to light, watery eyes, blurred vision, tenderness/swelling/stiffness of joints and or the neck/back, pustules on palms of hands or sole of feet.

The term “therapeutically effective amount”, used herein interchangeably with any of the terms “effective amount” or “sufficient amount” of a compound, e.g. a vaccine as described herein, is a quantity or activity sufficient to, when administered to the subject affect beneficial or desired results, including clinical results, and, as such, an effective amount or synonym thereof depends upon the context in which it is being applied. A human therapeutic vaccine is particularly described which is administered to a human being in an effective amount. A (human) therapeutically effective amount may be sufficient to treat, prevent, modulate, attenuate, reverse or inhibit a diseases or disorder associated with P. acnes. A therapeutically effective amount may be used for any prophylactic or therapeutic treatment.

The amount of the compound that will correspond to such an effective amount will vary depending on various factors, such as the given immunogen, the pharmaceutical formulation, the route of administration, the type and severity of disease or disorder, the identity of the subject or host being treated, and the like, but can nevertheless be routinely determined by one skilled in the art.

An effective amount of an immunogen as described herein, such as provided to a human patient at risk of developing a disease condition associated with a P. acnes infection, may specifically be in the range of 0.1 μg to 5 mg per antigen per dose.

An effective amount of the antigen as described herein, such as provided to a human patient in need thereof, may specifically be in the range of 0.5-1000 μg, preferably 1-500 μg, even more preferred up to 300 μg, up to 200 μg, up to 100 μg or up to 10 μg, though higher doses are indicated e.g. for treating acute disease conditions. Dosage treatment can be a single dose schedule or a multiple dose schedule. Multiple doses may be used in a primary immunization schedule and/or in a booster immunization schedule. In a multiple dose schedule the various doses may be given by the same or different routes, e.g. a parenteral prime and mucosal boost, a mucosal prime and parenteral boost, etc.

A treatment or prevention regimen of a subject with an effective amount of the antigen as described herein may consist of a single administration, or alternatively comprise a series of applications. For example, the immunogen may be administered at least once a year, at least once a half-year or at least once a month.

For example, the immunogen may be administered as a first dose followed by one or more booster dose(s), within a certain timeframe, according to a prime-boost immunization scheme to induce a long-lasting, efficacious immune response to P. acnes infection. A preferred administration schedule would encompass three or four doses, e.g. with an interval of 14 to 42 days between each dose.

Dosing treatment can be a single dose schedule or a multiple dose schedule. Multiple doses may be used in a primary immunization schedule and/or in a booster immunization schedule. Suitable timing between priming doses (e.g. between 2-16 weeks), and between priming and boosting can be routinely determined. For example, the minimum interval between the first and second vaccine dose can be 2 weeks, and the maximum can be 6 months. Recommended interval for additional doses (boosting) can be from 8 weeks up to 5 years.

The length of the treatment period depends on a variety of factors, such as the severity of the disease, either acute or chronic disease, the age of the patient, the concentration and the activity of the antibody format. It will also be appreciated that the effective amount used for the treatment or prophylaxis may increase or decrease over the course of a particular treatment or prophylaxis regime. Changes in dosage may result and become apparent by standard diagnostic assays known in the art. In some instances, chronic administration may be required.

The invention moreover provides pharmaceutical compositions, such as vaccine preparations which comprise an immunogen as described herein, and a pharmaceutically acceptable carrier or excipient.

As used herein, “pharmaceutically acceptable carriers” includes any material which, when combined with an active ingredient of a composition, allows the ingredient to retain biological activity and preferably does not cause disruptive reactions with the subject's immune system. In any way, all pharmaceutical formulations as disclosed and referred to herein must be provided and administered in a form which is acceptable under the laws and standards to be applied in the major markest, such as the EU or US. Especially, the formulations have to be provided under good manufacturing gpractice (GMP) rules and must not contain substances in an amount which is—if administered in the appropriate way—dangerous or creates an unacceptable risk for a human vaccination patient. The “human pharmaceutically acceptable carrier” is particularly compatible with the immune system of a human being. Pharmaceutically acceptable carriers generally include any and all suitable solvents, dispersion media, coatings, antibacterial and antifungal agents, isotonic and absorption delaying agents, and the like that are physiologically compatible with an antigen or antibody as described herein. Further examples of pharmaceutically acceptable carriers include sterile water, water for injection, saline, phosphate buffered saline, dextrose, glycerol, ethanol, and the like, as well as combinations of any thereof. The pharmaceutical composition can also contain one or more anti-caking agents, preservatives such as thimerosal or which are otherwise suitable for the proposed mode of administration, stabilizers such as amino acids and sugar moieties, sweetening agents such sucrose, lactose or saccharin, surfactants, pH buffering agents and pH modifiers such sodium hydroxide, hydrochloric acid, monosodium phosphate and/or disodium phosphate.

Specifically preferred embodiments of the pharmaceutical vaccine compositions comprise the polypeptide(s) according to the present invention in a buffer system, preferably in a phosphate buffer system. This phosphate buffer may preferably comprise NaH₂PO₄, Na₂HPO₄, KH₂PO₄, citric acid, especially mixtures thereof. Preferably chloride ions are present, especially as NaCl or KCl salts. Preferred amounts of these substances in a liquid formulation are: 0.01 mg/ml to 50 mg/ml, preferably 0.05 to 5 mg/ml, especially 0.1 to 1 mg/ml, polypeptide antigen(s) according to the present invention, 0.1 to 100 mM, preferably 0.5 to 50 mM, especially 0.1 to 10 mM, NaH₂PO₄, 0.1 to 100 mM, preferably 0.5 to 50 mM, especially 0.1 to 10 mM, Na₂HPO₄, 0.1 to 100 mM, preferably 0.5 to 50 mM, especially 0.1 to 10 mM, KH₂PO₄, 1 to 1000 mM, preferably 5 to 500 mM, especially 50 to 300 mM, NaCl, 0.1 to 100 mM, preferably 0.5 to 50 mM, especially 0.1 to 10 mM, KCl, and/or 0.01 to 1%, preferably 0.05 to 0.5%, especially 0.1 to 0.5%, aluminium adjuvant (aluminium hydroxide).

A preferred pH of a liquid formulation (or a reconstituted (e.g. with water for injection) formulation from dried (e.g. after lyophilization) or frozen preparations) of the pharmaceutical compositions according to the present invention is a pH of 4.5 to 9.0. If the composition contains a PITP polypeptide (PITP, fragments or derivatives thereof), the pH is preferably lower than 7.4 (the pH of blood), e.g. in the range of 4.5 to 7.4, preferably of 4.7 to 7.3, even more preferred of 5.0 to 7.3, especially of 5.3 to 7.2, to optimize stability (especially against higher temperature), solubility and prevention of aggregate formation.

Whereas in some embodiments, it may be advantageous to provide the PITP polypeptides and/or the porphyrin-binding PITP fragments and/or the porphyrin-binding PITP derivatives in a form which lack the porphyrin-binding property (e.g. based on manufacturing advantages), PITP surprisingly turned out to have a naturally binding affinity for prophyrins and related molecules. This explains some investigations in the prior art concerning in vivo porphyrin production and metabolism in P. acnes and the role thereof in acne patients and acne treatment (Schaller et al., Br. J. Dermatol. 153 (2005), 66-71; Shu et al., Curr. Med. Chem. 20 (2013). 562-568; Borelli et al., Acta Derm. Venerol. 86 (2006). 316-319).

According to a preferred embodiment of the present invention, a pharmaceutical preparation is provided which comprises PITP and/or a porphyrin-binding PITP fragment and/or a porphyrin-binding PITP derivative with a porphyin molecule bound to the PITP and/or a porphyrin-binding PITP fragment and/or a porphyrin-binding PITP derivative. In this embodiment, the PITP fragment and the PITP derivative contain—besides the at least one P. acnes PITP epitope—at least one porphyrin binding polypeptide, i.e. a polypeptide comprising an ENFD domain and/or a HbD domain or a porphyrin-binding fragment of the ENFD or HbD domain whereto a porphyrin molecule is bound in the formulation. In a preferred embodiment, the number of porphyrin molecules bound per PITP polypeptide, fragment or derivative is two porphyrine molecules per PITP polypeptide, fragment or derivative, i.e. with two porphyin-binding domains per molecule. Due to the similarity in binding affinity, besides porphyrins also porphyrinogens or porphyrin/porphyrinogen degradation molecules can also be bound to the ENFD and/or HbD domains in the polypeptides according to the present invention. The pharmaceutical preparation preferably contains a porphyrin, preferably hemin (as standard iron carrier and catalytic co-factor), protoprophyrin IX (as direct heme precursors, natural porphyrin of PITP), coproporphyrin I, II and/or III (preferably coproporphyrin III with its pro-inflammatory effect on keratinocytes; Schaller et al., Br. J. Dermatol. 153 (2005), 66-71), penacarboxyporphyrin, uroporphyrin, heptacarboxyporphyrin, hexacarboxyporphyrin (all intermediate porphyrins, all preferably form I or III), nitro-porphyrins, such as 5,10,15,20-tetraphenylporphyrin (TPP); 5,10,15,20-tetra(4′-fluorophenyl)porphyrin (TpFPP); 5,10,15,20-tetra(4′-chlorophenyl)porphyrin (TpClPP); 5,10,15,20-tetra(4′-bromophenyl)porphyrin (TpBrPP); which are synthetic and have anti-inflammatory, anti-arthritic and antinociceptive effect); a porphyrin degradation product which can be bound by the porphyrin-binding domain (such as biliverdin, the heme degradation product with significant anti-inflammatory effect, a.o. by antagonizing the pro-inflammatory phenotype of macrophages), 5,10-diphenyl-15,20-di(N-methylpyridinium-4-yl)porphyrin (Di-cis-Py+; a photosensitizer with antiinflammatory and antiproliferative activities), PORF-1 to PORF-34 (porphyrins with anti-inflammtory activity, e.g. inhibiting Fyn-kinase and in consequence inhibiting leukocyte proliferation and TNF-alpha production in leukocytes; Jelic et al., Europ. J. Pharmacol. 691 (2012), 251-260), porphine, octaethylporphyrin, tetraphenylporphyrin, verteporfin, ascoproporphyrin, hydroxymethylbilane (HMB), mesoporphyrin IX, 7-carboxyporphyrin (7P), 6-carboxylporphyrin (6P), heme A, heme B, heme C, heme O, heme I, heme m, heme D, S heme; and/or reduced versions thereof (porphyrinogens, such as uroporphyrinogen III), and/or chlorophylls, preferably chlorophyll a, chlorophyll b, and/or bacteriochlorophylls. Preferably, each of the porphyrines, porphyrinogens and chlorophylls are present in the formulation in a form complexed with one or several metal ions, more preferred with their natural metal ion or with a Fe-ion, such as a Fe (a siderophore, e.g. Fe²⁺, preferably Fe³⁺), Co, Zn, Mg, Se, Cu ion or mixtures thereof.

Additional pharmaceutically acceptable carriers are known in the art and described in, e.g. REMINGTON'S PHARMACEUTICAL SCIENCES. Liquid formulations can be solutions, emulsions or suspensions and can include excipients such as suspending agents, solubilizers, surfactants, preservatives, and chelating agents.

In one such aspect, an immunogen can be combined with one or more carriers appropriate for a desired route of administration, and may be, e.g. admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, polyvinyl alcohol, and optionally further tabletted or encapsulated for conventional administration. Alternatively, an immunogen may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. A carrier may include a controlled release material or time delay material, such as glyceryl monostearate or glyceryl distearate alone or with a wax, or other materials well known in the art. In a preferred embodiment, the pharmaceutical formulations according to the present invention are free of barium ions, citric acid compounds, such as citric acid monohydrate, formaldehyde, glutaraldehyde, and/or viral inactivators, such as beta-propiolactone (although these components are, in principle, allowed according to the standards of the Institute for Vaccine Safety (https://www.vaccinesafety.edu/Components-Excipients%2021-0115.pdf)) as vaccine components).

Suitable pharmaceutical compositions of an immunogen may be a vaccine. The terms “vaccine” or “vaccine composition”, which are used interchangeably, refer to pharmaceutical compositions comprising at least one immunogen or immunogenic composition that induces an immune response in an animal or human.

In general, the pharmaceutical composition or vaccine can be prepared in various forms, such as sterile solution, emulsion, suspensions, granules, tablets, pills, suppositories, capsules (e.g., adapted for oral delivery), patches, microbeads, microspheres, liposomes, salves, lotions and the like. The pharmaceutical composition or vaccine itself can be a freeze-dried or lyophilized vaccine reconstituted utilizing a physiologically acceptable buffer or fluid.

A preferred pharmaceutical formulation according to the present invention is a liquid formulation of phosphate buffered saline which can be stably stored at 2-8° C. For longer storage times, the sterile solution of the pharmaceutical formulation is aseptically filled into glass vials sealed with PTFE coated rubber stoppers and stored at −20±5° C. Stability data from the clinical batches of the formulations according to the present invention confirmed that ICH guidelines were fulfilled and are additionally supported by a stability program on GMP like batches, previously manufactured in the same way and used in the GLP toxicology program in rabbits.

Historically, live-attenuated or inactivated forms of microbial pathogens (viruses, bacteriae, etc.) have been used for induction of antigen-specific responses that protect the host against subsequent infections. Based on the pathogen being used, such vaccine formulations can contain anywhere between tens of to a few hundred proteins. However, protective immunity is usually dependent upon a few select proteins within such formulations, whereas the majority of proteins are unnecessary for the induction of protective immunity. Furthermore, these additional proteins may induce allergenic and/or reactogenic responses.

Polypeptide-based vaccine approaches include subunit vaccines; those consist primarily of shorter or longer polypeptide and can face limitations with respect to immunogenicity and thus may require multiple immunizations to achieve levels of immune response like inactivated or live attenuated pathogens. Nonetheless, a variety of approaches to enhance subunit vaccine responses have been utilized, including presentation of epitopes in multimeric format (e.g. virus-like particles, VLPs, or nanoparticles) or use of immunostimulatory adjuvants. The goal is to elicit peptide-specific B- or T-cell responses. It has to be noted that for efficient induction of either B-cell or cytotoxic T cell responses, the induction of a robust helper T cell responses is crucial.

First and foremost is the identification of immuno-dominant domains of epitopes that are capable of inducing protective immune response in terms of humoral immunity and/or cell mediated immunity against desired antigen. Immunodominant epitopes can be chosen in context of B cells, cytotoxic or helper T cells.

For obtaining immunodominant epitopes, a common strategy is to utilize naturally occurring antibodies or T-Cell-Receptors (TCRs) as a template for vaccine design, following the logic that if a particular epitope has already elicited a B- or Tcell response during natural disease, then it is sufficiently immunogenic to allow induction of similar responses by administration of a vaccine. In other cases, epitopes that elicit an immune response most favorable for mitigating the disease may not be the most immunodominant, and thus vaccination with critical epitopes may skew the immune response to yield protective responses. Therefore, vaccine programs shall seek to focus the immune response on the most conserved epitopes, and those that represent sites of susceptibility for virus neutralization.

In typical peptide vaccination protocols, the epitope of interest is conjugated to a carrier protein or presented in a multimeric format (VLP or nanoparticle). Such strategies can boost immune responses by increasing the half-life of the epitope by decreasing renal clearance and susceptibility to proteolytic degradation. Linkage to carrier proteins is typically achieved by chemical conjugation. The carriers are generally known to have immunogenic properties, and thus the simple covalent linking of epitopes to immunogenic species can often be sufficient to enhance the immune response. Related to this, the immunogenicity of peptide or protein sequences can be augmented through linkage to short sequences that are known to stimulate an immune response. An example of this is PADRE, a universal helper T-cell epitope that can be fused to peptide or protein sequences to stimulate antibody responses.

For the most part, protective antibodies target epitopes that lie on the surface of the pathogen (e.g. the viral glycoprotein or bacterial capsid). The targeted epitope, which is bound by the antigen-binding fragment (Fab) region of the IgG, often is a site of susceptibility for “neutralization by antibodies. In addition, both neutralizing and non-neutralizing pathogen specific antibodies may induce a number of immune mechanisms via the antibody Fc region that result ultimately in the destruction and/or clearance of the pathogen or pathogen-infected cell.

Generally, the elicitation of protective antibodies (B-Cell Responses) requires affinity maturation from the germline, a process that is stimulated by cross-linking B-cell receptors BCRs on a specific B-cell. To this end, monomeric peptides are of the poorly immunogenic relative to those corresponding sequences on viral, bacterial, or parasitic external proteins because, when presented in those contexts, multiple copies of the epitope on the pathogen surface permit efficiently cross-linking BCRs and thus stimulate antibody affinity maturations. One strategy to improve immunogenicity is to link the desired peptide epitope to a virus-like particle (VLP) or nanoparticle to allow ordered, multivalent epitope presentation that can more efficiently cross-link BCRs. Besides, also fragments of toxins or inactive variants of these can themselves be candidates for vaccines.

New polypeptide based vaccines must also consider promoting peptide secondary structure in order to induce a specific humoral response (T-Cell Responses).

Epitope specificity for T-cells is mediated by the T-cell receptor (TCR), which binds peptides presented in the “peptide binding groove” of class I or class II major histocompatibility complexes (MHCs, also known as human leukocyte antigen, HLA, for humans) on antigen presenting cells (APCs). Whole antigens are internalized and proteolyzed by APCs, and then short peptides (usually 8-11 residues in length for class 1, and 11-30 residues in length for class II) are loaded into MHCs (or HLAs) and presented on the APC surface. TCRs that are specific for the peptide epitope then bind those peptide-MHC complexes (pMHC), and a variety of proteins at the T-cell/APC interface orchestrate expansion of that T-cell clone.

Polypeptides presented in class I MHCs are typically short; class I MHC peptides follow a sequence pattern of X-(L/I)-X₍₆₋₇₎-(V/L), where L/I and V/L represent residues whose side chains anchor the peptide to the pMHC and thus are oriented toward the interior of the peptide binding groove and away from the TCR. The other positions point toward the TCR, and interactions with these residues mediate the epitope specificity. The sequences of class II MHC peptides are more varied but also contain anchor positions. The epitope peptide backbone binds snugly in the peptide binding groove with a extended backbone conformation, although bulging is accommodated for longer peptides in both class I and II MHCs. Furthermore, recognition of peptides requires a free N-terminal amine group.

Polypeptides that are loaded into MHCs or HLAs must conform to the above sequence requirements, but this does not guarantee that a particular epitope will be immunogenic. Nonetheless, the presentation of known immunogenic sequences can be accomplished by simply loading peptide repeats onto APCs such as dendritic cells. Also, systemic delivery of the polypeptides themselves or DNA encoding the epitopes is sufficient to stimulate T-cell expansion in vivo.

Interactions between proteins at the T-cell interface are generally clustered, and thus individual protein-protein interactions, including those between pMHC and the TCR, or PD-1 and its primary ligand, PD-L1, are low affinity (KD˜micromolar range) when measured using soluble forms of each component. Interactions between the peptide-binding platform of the MHC and TCR are central to the T-cell/APC interface, and thus TCRs cannot recognize their peptide epitopes without epitope presentation in this format. Furthermore, the antigen specificity of the T-cell is dependent on the TCR-pMHC interaction, and thus the structural features of the epitope-MHC-TCR ternary complex can be an important consideration for T-cell targeted vaccines. Recognition of particular TCRs on cells using soluble peptide-loaded MHC (pMHC) protein requires presentation of the pMHC in a multivalent fashion. This is most commonly achieved by biotinylation of the pMHC and subsequent complexing with streptavidin, which provides 3-4 pMHCs per streptavidin molecule. Folding of MHCs is dependent on the peptide; thus exogenous expression of pMHCs typically involves fusion of the peptide epitope to the MHC using a polypeptide linker. A number of in vitro and chemical methods have also been devised to allow exchange of the bound peptide with exogenously added peptides.

An example are peptides recognized by CD8 T cells. The synthetic peptides used were often longer than the 9-11 amino acids of the minimal peptide-sequence recognized by CD8 T cells. The longer peptides need to be trimmed to minimal MHC-I binding ligands by proteases and peptidases or by professional Antigen Presenting Cells (APC) process, followed by loading onto MHC-I groves. In fact, minimal peptides used in most of studies induce lower immune responses compared to longer peptides, since they only elicit CD8 T cell response without processing by APC. Furthermore, it is suggested that a peptide vaccine should consist of multi epitopes, which could include the MHC I1 restricted helper epitopes recognized by CD4 T cells and MHC I restricted CD8 epitopes to induce both helper T cells and cytotoxic T cells and humoral responses.

As discussed above, T-cell epitope backbone conformations (epitope structures) are limited by the steric restriction of binding into the MHC peptide binding groove, but antibody epitopes can be much more heterogeneous in conformation. Antibodies that are specific for linear peptide sequences typically contain a groove at the combining site, whereas those that bind protein surfaces that span multiple secondary structural elements are generally flatter. Peptide epitopes can bind antibodies in α-helical, β-strand/extended, or loop conformations. The precise conformation that the peptide epitope adopts in the antigen-antibody complex can sometimes be important for the activity of the antibody. In these cases where structure is thought to be an important aspect, the presentation of peptide vaccines in a conformationally relevant manner then becomes a key factor for vaccine design. Conformational dependence of the epitope may be important because it allows recognition of the epitope by the antibody within the larger context of the globular antigen fold.

The clearance of an infection may also require a robust and cross-reactive CD4 and CD8 T-cell response as well as neutralizing antibodies. Identification and characterization of cytotoxic T lymphocytes (CTL) epitopes as well as broadly neutralizing antibodies that target conserved epitopes of microbial surface has prompted the exploration of peptide-based vaccine strategies.

A new strategy is the complexing of a scaffold, e.g. monoclonal antibody with the epitope (linear epitope, cyclic peptides that e.g. use a beta-hairpin structure etc.), a spacer may also be included.

A next generation approach may include polypeptides liked to a helper T-cell epitopes derived from the highly antigenic measles virus fusion protein (MVF 288-302) and Hepatitis B virus surface antigen (HBsAg19-33). Sites within these epitopes were optimized by combinatorial mutagenesis and selected for broad responsiveness in genetically diverse backgrounds. The peptides then were mixed in a n equimolar ratio with polyanionic CpG oligodeoxynucleotides to form stable micrometer-sized particulates mediated by electrostatic interaction.

Cyclic, conformational peptides of specific sequences may be engineered with different disulfide pairings.

Polypeptides may also be conjugated to an amphiphilic lipid that directs the target epitope to lymph nodes. These so-called “amph-ligands” contain a bifunctional distearoyl phosphoethanolamine, which binds albumin and can also insert into cell membranes as well as either a peptide or small molecule antigen attached by a PEG linker. The amph-ligands accumulated in the lymph nodes and readily inserted into the membrane of dendritic cells.

Most vaccines are injected with an adjuvant to stimulate an immune response. The nature of adjuvants can vary extensively and is an important consideration for peptide vaccination studies. For example, conformationally designed epitopes may require adjuvants that do not denature or emulsify the antigens.

A possible synthetic adjuvant is poly-L-arginine. A possible adjuvant are emulsions. Emulsions can be single (oil-in-water (o/w), water-in-oil (w/o) or multiple (e.g., water-in-oil-in water w/o/w) and the stability of emulsions as delivery systems is directly proportional to vaccine safety and efficacy. Various modifications of existing emulsion based delivery systems have promising future, e.g., NH₂ containing mineral oil and high purity oleic acid derivative, sorbitan monooleate, squalene oil, DepoVax (liposome containing the adjuvant and antigen is suspended in oil), and GLA-SE (glucopyranosyl lipid adjuvant-stable emulsion). A second example are the liposomes, phospholipid bilayer structures that form small vesicles mimicking cell membranes. The phospholipid constituents include cholesterol. A method for formulation is to conjugate the peptide moiety with lipids followed by its incorporation in liposomes. The immunostimulating effect of liposomes is mediated by the protection of antigens against proteolytic enzymes. They are also known to extend the half-life of antigens in blood so that a maximum exposure of antigens to APCs occurs. Liposomes can be made positively charged (cationic liposomes), coated with polyethylene glycol (PEGs) to promote their interaction with APCs. Liposomes can also be made pH sensitive or integrated with fusogenic peptides to deliver the peptide vaccine into the cytosol and promote the CTL response. A novel approach is to add an ER insertion signal sequence (Eriss) into the fusogenic liposomes to promote the peptide-MHC class I association for enhanced peptide transportation into the endoplasmic reticulum (ER).

A similar group of colloids to liposomes that has been explored for the delivery of antigens are virosomes, transfersomes, archeosomes, niosomes and cochleates. Niosomes are made of non-ionic surfactants and are considered to be more stable than conventional liposome. Virosomes are composed of assembled viral membrane protein which render them enhanced binding to APCs and promote cytosolic delivery. Virosomes are excellent adjuvant systems and are biodegradable, non-toxic, and do not induce antibodies against themselves. Immunostimulatory complexes (ISCOMs) are particulate antigen delivery systems composed of antigen, cholesterol, phospholipid and saponin and around 40 nm size. ISCOMATRIX™ is a particulate adjuvant comprising cholesterol, phospholipid and saponin but without antigen.

ISCOMs and ISCOMATRIX™ are composed of phospholipids as liposomes but also contain saponin adjuvant Quil A. ISCOMS can only be loaded with hydrophobic antigens. Strategies to encapsulate hydrophilic antigens into ISCOMS include: coupling of antigens to ISCOMs using amphipathic coupling protein; conjugation of hydrophilic with fatty acids and phospholipids; and, modification of protein by genetic engineering. ISCOMSs are known to induce CTL responses for native as well as modified immunogens and can mediate humoral as well as cell-mediated immune responses.

In recent years various polymers have been investigated for the delivery of the vaccines. The natural polymers available for the production of nanoparticles include albumin, collagen, starch, chitosan, dextran, whereas the examples of synthetic polymers include polymethylmethacrylate, polyesters, polyanhydrides, and polyamides. Of the synthetic polyesters, polylactides (PLA), polyglycolides or polyglocolic acid (PGA) and their copolymers poly(lactide-co-glycolide) PLGA are US FDA approved for use in humans and have been tested for toxicity and safety in extensive animal studies [3,92]. The popular choice for biodegradable polymers are aliphatic polyesters such as poly(lactic acid) (PLA), poly(glycolic acid) (PGA), poly(e-caprolactone) (PCL), poly(hydroxybutyrate) (PHB) and their copolymers. In particular, poly(lactide-co-glycolide) (PLGA) has been the most extensively investigated for developing nano- and microparticles encapsulating therapeutic drugs in controlled release applications.

Owing to their particulate nature polymeric micro and nanoparticles are known to promote uptake, transport, or presentation of antigen to APCs. They were also found to elicit both cellular and humoral immunity. The biggest advantage offered by polymer based antigen delivery systems is the sustained release (for a period of few weeks to months) of the encapsulated antigen from the polymer matrix. The rate of release of the antigens from the encapsulated polymeric particles can be controlled by the rate of degradation of the polymer matrix which, in turn, is dependent on the composition of the polymer matrix, molecular weight of the polymer and size of the particles. Generally, hydrophobic interactions, electrostatic forces, hydrogen bonds, van der Waal forces, or combinations of these interactions are available as the driving forces for the formation of the polymer complexes. The natural polymer, chitosan, has been known to enhance the bioavailability of the antigens due to a mucoadhesive property. A possible contributing mechanism is that chitosan has been shown to relax intercellular tight junctions and improve the paracellular transport of antigens.

Other particulate systems used to deliver vaccine antigens include carbon nanotubes, silicon dioxide nanoparticles, dendrimers [100], ferritin nanoparticles, peptide nanocarriers, gold nanoparticles, liposome-polycation-DNA (LPD) complex, oligosaccharide ester derivatives (OEDs) microparticles and combination systems, e.g., liposomes and w/o emulsion.

Safety of the particulate delivery strategies is of highest concern when selecting the particular strategies for delivering the peptide antigen. The route of administration from which the particulate delivery system is administered plays a vital role in toxicity determination. The common routes of administration of the vaccines are subcutaneous, intranasal, intravenous, and transdermal.

For intramuscular (i.m.) and for intradermal (i.d.) application, the vaccine product according to the present invention is provided in a ready to use, physiologic PBS solution. An intradermal, transdermal or a subcutaneous administration is performed using a variety of methods, including intradermal injection applicators, microneedles, transdermal laser devices, skin patch or other suitable skin-adapted application procedures.

Administration of the pharmaceutical composition comprising an antigen or polypeptide as described herein, may be done in a variety of ways, including orally, subcutaneously, intravenously, intranasally, intraotically, transdermally, mucosal, topically, intraperitoneally, intramuscularly, intranodal, intrapulmonary, e.g. employing inhalable technology or pulmonary delivery systems, vaginally, parenterally, rectally, or intraocularly.

Exemplary formulations as used for parenteral administration include those suitable for subcutaneous, intramuscular or intravenous injection as, for example, a sterile solution, emulsion or suspension.

These pharmaceutical compositions or vaccine compositions can be administered in accordance with the present invention as a bolus injection or infusion or by continuous infusion.

Administration of the pharmaceutical vaccine formulations to the human patients may be performed by any suitable means, preferably by syringes. Other preferred means for administration are modem administration devices, such as microneedle patches, microinjection devices, and high density microarray patches (as disclosed in Prausitz et al., Curr. Top. Microbiol. Immunol. 333 (2009). 369-393 (microneedle patches with dissolvable microneedles); NanoPass Technologies (MicronJet600: a 0.6 mm intradermal microinjection device); high density microarray patch (HD-MAP). 10×10 mm square with more than 3000 micro-projections that are gamma-irradiated before aseptic dry application of vaccine to the HD-MAP's tips by Vaxxas (Australia), Forster et al., PLoS Med 17 (2020), pmed.1003024; DBV Biotechnology (Viaskin technology): https://www.dbv-technologies.com/viaskin-platform/).

Preferably, the P. acnes polypeptides disclosed herein may be provided in a pharmaceutical composition, especially a vaccine composition by recombinant production processes wherein the polypeptide is expressed in suitable host cells which are genetically programmed (“transformed”) to express this polypeptide (which is usually not naturally expressed by the host cell). In the course of this recombinant production/fermentation process, it is known in the present field that the N-terminal methionine residue is lost before the final preparation of the polypeptide from the fermented host cell culture. This loss/presence of the N-terminal methionine residue may be caused by the presence of a methionine aminopeptidase (the enzyme which removes methionine, or specifically formylmethionine from the N-terminus; see e.g.: Xiao et al, Biochemistry 49 (2010), 5588-5599) in the process of expression and purification of the (recombinant) polypeptide. Technically it is also possible that a certain fraction of polypeptides does not contain methionine but formylmethionine, especially in prokaryotic host cells. Depending on the specific expression system, this may be the actually initiating amino acid, but it is typically enzymatically altered by enzyme formylmethionine deformylase to standard methionine, which can then be cleaved off (or not; Wingfield, Curr. Protoc. Protein Sci. 88 (2018), 6.14.1-6.14.3). Both (methionine and formylmethionine) may be cleaved by methionine aminopeptidase. Normally this formylation should affect only a minor fraction of a recombinant polypeptide lot produced by a fermentation process, but since this is a statistical process, a certain fraction of polypeptides may start with formylmethionine rather than methionine. Accordingly, all sequences disclosed herein are to be regarded as being disclosed with or without the N-terminal methionine residue, especially with respect to the absence of this N-terminal methionine residue in the final pharmaceutical composition which is then applied to the human patient. Accordingly, this N-terminal methionine residue may be present or not in a polypeptide being recombinantly produced in a given host according to the present invention (with an expression system encoding this N-terminal methionine in principle) so that compositions are preferred wherein the P. acnes polypeptides according to the present invention are provided which at least partially lack an N-terminal methionine residue (although in principle encoded by the expression system). Moreover, the starting methionine can also be simply added to a fragment or derivative of the present invention (which fragment or derivative would not contain an N-terminal methionine but which is added for expression purposes).

Moreover, also expression systems can be provided which do not need an AUG codon/initial methionine as starting signal for recombinant polypeptide generation (e.g. flaviviruses with an IRES or Plautia stali intestine virus (Sasaki et al., PNAS 97 (2000), 1512-1515)). There are also cellular IRES' (Yang et al., J. Mol. Cell Biol. 11 (2019), 911-919) which does not even require use of viral expression systems in eukaryotic cells (also possible for prokaryotic cells: Colussi et al., Nat. 519 (2015), 110-113).

In one embodiment, the antigen as described herein is the only therapeutically active agent administered to a subject, e.g. as a disease modifying or preventing monotherapy.

Alternatively, the antigen as described herein is administered in combination with one or more other therapeutic or prophylactic agents, including standard treatment, e.g. antibiotics, topical or systemic retinoids, steroid and non-steroid inhibitors of inflammation, and/or other antibody-based therapy, e.g. employing anti-bacterial or anti-inflammatory agents.

As already stated above, the vaccines according to the present invention may be provided in the form of nucleic acid vaccine comprising antigen encoding DNA or RNA molecules.

Preferably, the vaccines according to the present invention are provided as mRNA vaccines. mRNA vaccines represent a promising alternative to conventional vaccine approaches because of their high potency, capacity for rapid development and potential for low-cost manufacture and safe administration. Stability and efficient in vivo delivery of mRNA has been achieved by recent technological advances leading to the successful vaccination approaches for SARS-COV-2 mRNA vaccines. Multiple mRNA vaccine platforms against infectious diseases and several types of cancer have demonstrated promising results in both animal models and humans for other diseases (Pardi et al., NRDD 17 (2018), 261-279). This review provides a detailed overview of mRNA vaccines and considers future directions and challenges in advancing this promising vaccine platform to widespread therapeutic use.

For the mRNA vaccine according to the present invention, the cDNA (used for production of the mRNA) is provided as a DNA construct suitable for biotechnological amplification, preferentially as a plasmid. Preferred plasmids include at least a DNA sequence for plasmide replication (ORI), optionally selective markers (such as antibiotic resistance genes) and an encoded antigen construct (encoding for a t least one DsA1/DsA2/PITP epitope as described herein). The antigen is under the control of a suitable promoter region for a DNA dependent RNA polymerase, preferentially recognized by T7 polymerase. Alternatively several genes may be encoded in a single cDNA construct, combining multiple promoter/antigen pairs in tandem. Alternatively several genes may beencoded in a single cDNA construct, where at least one may be intended for modifying the immune response against the encoded antigen, where examples include Interleukins such as IL-10 or IL-2. The preferred structure of the transcribed cDNA which is equivalent to encoded mRNA is (after—preferably—a 5′ CAP) 5′UTR, eventually a signal peptide (SP), the encoded antigen and 3′UTR, preferably with a polyA tail ((5′ CAP)-5′UTR-(SP)-antigen/epitpe-3′UTR-(ployA)). In an alternative approach, also a replicase is provided, typically of viral origin, to amplify the mRNA in the cytoplasm. This replicase may be encoded between 5′ UTR and signal-peptide, but can also be provided as a second RNA molecule. The cross-type-reactive antigens and/or epitopes may be provided in all practically relevant forms of mRNA vaccines.

The 5′ UTR function is primarily to initiate translation of SP joined to the encoded antigen, minimally, a Kozak fragment. Many alternatives are possible featuring various optimizations. The Kozak fragment can also be replaced by an alternative means of initiation of translation, such as an IRES (internal ribosome entry site) of viral or eukaryotic (and especially human) origin. Prominent variants are listed in the sequence listing (e.g. SEQ ID NOs:65-77). The 5′ UTR can preferentially be 5′ capped, preferentially by 5′ addition of a N7-methyl guanosine (m7G cap or m7Gppp) and/or an additional methylation on the 2′O position (m7GpppNm cap).

The “encoded antigen” is the actual antigen/epitope sequence, without a signal peptide. Preferably, these sequences may be optimized and non-optimized DNA sequences encoding the epitope containing peptides (e.g.: Mauger et al., PNAS 116 (2019), 24075-24083; Holtkamp et al., Blood 108 (2006), 4009-4017). For example, the epitope/antigen sequence is optimized to increase secondary structure formation, preferably also using modified nucleotides, enhancing mRNA half-life. These changes affect mRNA half-life and in consequence overall expression. For example, uracil bases can be replaced by alternative bases, including but not exclusive to pseudouridine and N-1-methylpseudouridine or equivalently coding (i.e. synonymous) codons featuring a higher GC content or codons affecting RNA secondary structure content of the coding sequence or pseudo-knots involving other parts of the construct. It is also possible to encode multiple antigens in one DNA/RNA sequence, using alternative frames, a form of polycistronic encoding. Practically, several distinct coding frames this can be joined by IRES to encode and provide several proteins serially. In the special forms of either cleaved or polycistronic constructs, a replicase can be added to the construct. This may be a replicase (primarily an RNA dependent RNA polymerase), especially of viral origin.

SP: optionally a eukaryotic signal peptide for extracellular localization of the encoded antigen is included. This is preferentially the IL-6 or functionally similar signal peptide. On the DNA/RNA level the signal-peptide may be specifically optimized to reflect typical codon, bicodon and RNA-structural biases of highly-expressed genes.

3′ UTR: The role of the 3′ UTR is primarily to facilitate mRNA stability (partially through the optional poly-A tail), but may also include elements for efficient termination of RNA polyerase activity (termination of transcription), u.a. to avoid artifacts such as joined transcripts. Various 3′ UTR expression optimizing features have been identified (Horstick et al., NAR 43 (2015), e48). The 3′ UTR can preferably be derived from the 3′ UTR of an existing human gene, preferably a highly expressed one; the 3′ UTR of a gene of a human RNA or DNA virus, preferably a highly expressed one; the 3′ UTR of an existing gene of a non-human species, preferably a highly expressed one; the 3′ UTR of a fully synthetic construct supporting maximal mRNA stability and protein expression, typically characterized through one of the following methods or a combination thereof.

The 3′ UTR can preferentially be 3′ polyadenosylated. It is general standard to include a poly-A tail at the 3′ end of the 3′ UTR, but this is not absolutely compulsory (Nicholson et al., Tr. Cell Biol. 29 (2019), 191-200). In almost all cases poly-A acts synergistically and in a co-dependent fashion with the 5′ CAP structure for mRNA stability and translation efficiency. Substantial poly-A tails are typically considered to be beneficial (specifically for mRNA stability), but under certain circumstances long tails are not an absolutely critical requirement for efficient translation and stability (Jalkanen et al., Semin. Cell Dev. Biol. 0 (2014). 24-32). Also, the 3′ UTR of metazoan histone genes is not polyadenylated and features a highly conserved RNA secondary structure. Alternatively to polyandenosylation a poly-A tail can also be encoded on DNA level before production, e.g. usually with 30 to 70 nucleotides in length (Holtkamp et al., Blood 108 (2006), 4009-4017).

Mixed tailing (i.e. composing the poly-A tail not exclusively of A) specifically using TENT4A and TENT4B enzymes has been shown to protect against rapid deadenylation (Kim et al., Nat. Struct. Mol. Biol. 27 (2020), 581-588). This primarily refers to G, possibly (but not shown to C) but not U. Tailing with U in the reverse promotes mRNA decay, i.e. decreases stability. This may only apply to poly-uridylation, the effect of single uracils is unclear based on literature, but as a trend it is destabilizing. Presumably in case the poly-A tract is already encoded in the cDNA this can be more specifically designed than using an enzymatic machinery.

The 17 nt limit is based on Xenopus albumin mRNA, which is similarly efficiently translated compared to longer polyadenylation. The 28 nt limit is quite low, but this is the lowest limit where A-A-U triple helices can be formed. Too-short poly-A tails are usually associated with 3′ uridylation and increased mRNA degradation. Similarly hyperdenylation is typically detrimental to RNA stability, presumably because it is a characteristic of ‘old’ mRNA which failed to export, so is a signal for further degradation. But this does not absolutely need to be the case, structural arrangement or PABPC binding can antagonize this effect, while it can be enhanced by miRNA binding.

In summary, including a poly-A tail of common size is preferred. The poly-A can be introduced by encoding it in the cDNA template or enzymatic addition using a poly(A) polymerase (PAP). Accordingly, the poly-A tail is preferably primarily or entirely composed of adenosine bases, but admixture of other nucleotides (specifically guanosine) leads to mixed tailing and can increase mRNA stability. Incorporation of a G preferredly within the last 10, more preferredly within the last 6 and most preferredly in the last position of the poly-A tail is preferred. Overall, the poly-A tail in a mixed tailing should preferably contain A comprising at least 95% and an alternative base (N) where N can be preferably G or less preferred Cytosine (C) or least preferred a synthetic nucleoside analogue where N comprises preferredly 1-2% of the tail, less preferredly 5% of the tail. Spacing between individual G nucleotides in the poly-A tail should be at least 20 nucleotides, preferably at least 30 nucleotides, but optimally less than 40 nucleotides. Preferably, the poly-A tail consists of at least 17 nucleotides in length, preferably at least 28 nucleotides in length. Preferred poly-A tails are 50-200 nucleotides in length, especially 100-200 nucleotides in length. If the 3′ UTR is substantially derived from a metazoan histone gene gene normally lacking polyadenylation, the poly-A tail may preferably be omitted. This may also be the case if the 3′ UTR is terminated with a Histone 3′ UTR stem-loop derived from a metazoan histone gene. Optionally, a 3′ UTR terminated by a histone 3′ stem-loop can be extended with a poly-A tail (which has been shown to further enhance stability). In RNA vaccines, longer tails (i.e. >200-250 nt) may even be more preferred compared to a DNA encoded antigen. A preferred option is to use histone 3′ UTR or the 3′ stem-loop.

Concerning sequence optimizations for mRNA vaccines, there are especially two strategies which may be highlighted: 1) Creating artificial cDNA sequences based on the bacterial antigen but representing human codon bias, i.e. using the most frequent codons for each amino-acid, but codon usage should reflect amino-acyl tRNA concentration, meaning that there is simply more of a particular tRNA to recognize a codon, meaning the codon is simply faster translated. This is the basic rationale of using organism codon bias for mRNA optimization. An analysis of human codons in protein coding genes suggests that GC-rich codons are typically more abundant than low GC codons, at least for for given amino-acid. This may be the reason high GC mRNA sequences tend to translate at significantly faster rates (up to 100 fold).

Preferred optimizations applied according to the present invention are mainly aimed for streamlining expression of included antigens. It is also preferred to optimize RNA sequence based on the codon usage of genes highly expressed in human skeletal muscle used to reverse synthesize an optimally expressible mRNA sequence (“CODON MUSCLE”) and a bicodon usage of top 2% genes highly expressed in human skeletal muscle used to reverse synthesize an optimally expressible mRNA sequence (“BICODON MUSCLE”). The (simple) Bicodon hybrid model applies a bicodon usage differentiating between the first 16 residues/codons and the rest of the sequence. This based on position dependent codon bias reported by some studies, i.e. particularly in highly expressed gene the N-terminal region tend to show shows different codon preferences compared to non-N-terminal regions. Reason may be a preference for low RNA structure close to the origin of transcription (which benefit from AT-rich sequences) while the remaining portion of the RNA may benefit from strong RNA secondary structure best achieved through GC rich sequences. This aspect has been implemented here by a simple model where the first 16 codons are selected from a model globally optimized for N-terminal codon usage, while the remaining residue (17-end of the protein) is optimized by a model based on homogeneous bicodon usage. This model is of particular use in cases where no signal peptide is used, as normally the N-terminal codon preference should mostly affect the signal peptide codon sequence. This also means that an optimization of the signal peptide makes sense, this may significantly affect expression. Here it has to be borne in mind that expression level of proteins is actually determined based on transcript levels. This is easier and more precise to determine than generated protein levels, but naturally does not directly reflect generated protein levels. For the “CODON MUSCLE” optimization codon usage of top 2% of human skeletal muscle expressed transcripts has been used as a basis, where no difference is made between the N-terminal region and the overall sequence. The “BICODON MUSCLE” optimization refers to the preferences of specific codons to neighbour each other, which can be different from the overall preference for specific codons. Whatever the biochemical reason for these biases, they can be used to create more highly expressible DNA/RNA sequences.

For example AAC-CGG is only the third or fourth most abundant bicodon for amino-acid sequence NR, with an abundance of 12.01% while the most abundant bicodon AAC-AGA is found in 16.88% of cases within our human muscle reference transcript dataset. This is interesting, since both AAC (N) and COG (R) are the individually most prominent codons in the same dataset, so without biases one would assume this combination to be the most prominent as well.

Since not in every position optimal bicodons are possible as they can be mutually exclusive (they always overlap by one codon with the previous and equally with the next bicodon), therefore a dynamic programming optimization strategy has been applied to propose a globally bicodon optimal sequence. According to the “Inhomogeneous model”, the N-terminal 16 residues and the rest of the sequence are treated differently. As previously explained, these sequences are differently bicodon-optimized in the N-terminus and the rest. For this the very simplest solution has been chosen, the sequence was first optimized with the N-terminal muscle bicodon profile, and then with the global muscle bicodon profile (but always based on top 2% transcripts). Then the first 16 codons of the N-terminal model were swapped with the first 16 generated with the global profile. This works similarly compared to a more subtle transition between models unless the transition point introduces a rare bicodon.

The course of optimization which can be derived from SEQ ID NOs:67-76 is not based on any particular gene set (although also e.g. generally tissue specific optimizations or generally highly expressed genes are possible and make sense), but is based on codon frequency of the longest transcripts of all human protein coding genes which also reflects the high GC rule well (Athey et al., BMC Bioinformatics 18 (2017), 391). It also makes sense to not use a single codon for a specific amino-acid, to avoid depletion of specific amino-acyl tRNAs, also dependent on the planned routes of administration. i.e. a trans-dermal vaccine may benefit from a different codon optimization than a mucosal or muscle administered one). 2) The second major aspect of optimization is to reduce the uracil content in mRNA, i.e. thymine (T) in cDNA. The reason is that in linear (single stranded) RNA uracils are recognized by part of the innate immune system specifically an RNA-dependent protein kinase (PKR), which in turn inhibits initiation of translation, with the macroscopically visible (measurable) effect of decreased translation of an mRNA. There are two solutions to this, one is recoding using low T/U codons, the other is to use certain uracil analogues such as pseudouridine and N-1-methylpseudouridine. These may either be incorporated by DNA dependent RNA polymerases (at least as far as these polymerases can deal with the analogues), or can be incorporated in the finished RNA by enzymatic modification. Both strategies, i.e. reducing the content by encoding and chemical modification can also be combined. More important than translation efficiency, and probably the major reason why mRNA therapeutics are only seeing adoption in very recent years mRNA can stimulate significant inflammatory reactions, which can to some degree be overcome by engineering the uracil content (Thess et al., Mol. Ther. 23 (2015), 1456-1464). Although optimization may not always be necessary chemical and sequence based optimization appear to be very significant recent break-though aspects in mRNA therapeutics and vaccines.

Further engineering possibilities include those affecting mRNA secondary structure with or without affecting the protein sequence, or those affecting translation efficiency but also protein sequence (e.g. mutating codons for which no synonymous high-GC or low U versions exist to one encoding chemically similar amino-acids but allowing high-GC low U content). Also codon independent dinucleotide optimization is another option. E.g. codons GGT and TCG together would form a TT sequence, i.e. although each has a lo U content together they would form an island of high uracil.

Examples for base optimization for preferred polypeptides of the present invention (a DsA1/DsA2 shuffle polypeptide (H4-V3) and a preferred PITP fragment (P028-V7)) were subjected to (1) basic optimization for translation in humans using codon-optimization, (2) following basic rules such as GC preference and low T/U content and (3) a slightly more subtle version basically penalizing T/U and taking codon preference into account (which typically also results in GC-rich codon preference). The results are displayed by SEQ ID NOs:67-76.

It is also possible to introduce point mutations into the polypeptide sequence to achieve beneficial effects on mRNA stability/translation potential/inflammatory potential. Naturally such mutations are preferably be rare and measured, i.e. one will typically mutate against compatible/similar amino-acids. The table below lists codon exchanges which have beneficial effects. Listed codons and alternative are the respective optimal for each amino-acid, based on (1) human codon bias, (2) high-GC+low uracil or (3) human codon bias+low uracil. PGP-2

| | | | | | | Count of | |
|---|---|---|---|---|---|---|---|
| Optimal | | | Count of | | | altern. | |
| codon by | | | codon in | Altern. | Altern. | codon in | Type of |
| criterion | Residue | Codon | genome | Residue | Codon | genome | improvement |
| 1, 2, 3 | Y | TAC | 165972 | W | TGG | 143644 | Higher GC |
| 1, 2, 3 | Y | TAC | 165972 | H | CAC | 175725 | Higher GC |
| 1, 2, 3 | F | TTC | 222375 | Y | TAC | 165972 | Reduce T/U |
| 1, 2, 3 | K | AAG | 364887 | Q | CAG | 401944 | Higher GC |
| 2 | K | AAG | 364887 | R | CGG | 133085 | Higher GC |
| 1, 2, 3 | I | ATC | 224955 | V | GTG | 314084 | Higher GC |
| 1, 2, 3 | I | ATC | 224955 | L | CTG | 449868 | Higher GC |
| 1, 2, 3 | N | AAC | 213224 | D | GAC | 288185 | Higher GC |
| 1, 2, 3 | M | ATG | 244660 | L | CTG | 449868 | Higher GC |
| 1, 3 | R | AGA | 143888 | Q | CAG | 401944 | Higher GC |
| 1, 2, 3 | S | AGC | 231831 | A | GCC | 323834 | Higher GC |
| 2 | Q | CAG | 401944 | R | CGG | 133085 | Higher GC |

The general principle of this preferred nucleotide exchange strategy is to exchange codons only if the new codon (encoding a new amino-acid) if the new amino-acid does not significantly disturb the antigenic character of the protein but significantly improves translation efficiency or significantly reduces uracil content. To pre-select for likely tolerated amino-acid exchanges, only those were further considered with a positive value in the BLOSUM90 matrix. This basically means that those amino-acids can be seen exchanged in highly similar proteins (i.e. without a lot of compensatory mutations, which argues for more compatible mutations). Regarding uracil, there is only one mutation: F (TTC)→Y (TAC) which may be beneficial by the applied metrics, but overall codon abundance of TAC is 25% lower than that of TTC, so the whether this is really an advantage in expression is ambivalent and depends on the effect of uracils. Regarding GC content maximization as well as improved frequency, particularly the following are preferred:
Y (TAC)→H (CAC) [higher overall codon number, higher GC content]
I (ATC)→L (CTG) [significantly higher overall codon number, higher GC content]
N (AAC)→D (GAC) [higher overall codon number, higher GC content]
M (ATG)→L (CTG) [significantly higher overall codon number, higher GC content]
S (AGC)→A (GCC) [significantly higher overall codon number, higher GC content]
R (AGA)→Q (CAG) [significantly higher overall codon number, higher GC content]

Again, also total codon abundance must be considered, i.e. that more abundant amino-acids should correlate with higher availability of a matching loaded tRNA. SEQ ID NOs:67-76 are sequences optimized for (1) human codon bias (2) high-GC+human codon bias (the latter mostly to select from GC content equally ranked codons).

As already disclosed above, to increase tolerogenicity/decrease specifically immunological side effects of RNA vaccines, mRNA is typically modified (i.e. typically all though not strongly necessarily comprising all described parts or the mRNA construct), a factor for safety and patient compliance. Notably this is a very different concept to normal vaccines, where the initial formulation already attempts to shape immunity, while in RNA vaccines the initial (RNA) form should be mostly immunologically inconspicuous, and only the translated (protein) form should give rise to immune responses.

RNA modifications can be included by transcription, i.e. by using modified nucleotides in the polymerization process (possibly requiring or benefiting from engineered DNA dependent RNA polymerases), or by enzymatic modification of the transcribed RNA. Both methods are in use. Know effective RNA modifications (nucleoside analogues/replacements) for mRNA vaccines include (Zhang et al., Front. Immunol. 10 (2019), 594; the “RNA Modification Database” https://mods.rna.albany.edu/mods/):
Uridine→Pseudouridine (symbol: Ψ)
Uridine→N-1-methylpseudouridine (mT)
Cytosine→5-methylcytosine (5mC)
Any other Uracil (base) analogue maintaining the coding potential and when included into an RNA sequence characterized by reduction of recognition by RNA-dependent protein kinase (PKR)
Any other natural or non-natural RNA base analogue which, when incorporated into mRNA (by transcription or chemical modification of exiting mRNA), still maintains the coding potential of the mRNA but reduces inflammation (e.g. by reduced recognition through TLR7 or TLR8) or mRNA degradation

Both in prokaryotes and in eukaryotes the first, N-terminal amino-acid critically determines protein stability. In cases where the N-terminal methionine is removed and encoded second residue becomes the first and further determines stability (“N-end rule”). These aspects are not independent, as particularly large or bulky residues limit or prevent methionine removal. Generally, methionine is a stabilizing residue, but valine leads to roughly 3-fold higher stability in mammals. As methionine can be removed when valine is the second residue a protein starting by residues “MV” (e.g. where the second position is mutated to valine) is generally more stable and hence more highly expressed than other versions. On DNA level this means that it is beneficial to start all reading frames by sequence ATG-GTG, where ATG encodes methionine, and GTG is the most frequent codon for valine. According to a preferred embodiment, therefore derivatives are used according to the present invention which contain an N-terminal “MV” sequence (on DNA level with initial coding DNA sequence “ATGGTG”) for all sequences intended for application as mRNA vaccine, along with the standard sequence. In addition, also mutation of acidic residues (Asp, Glu) in positions 3 and 6 can enhance removal of the initial methionine and in consequence enhance stability if the second residue is valine. Target residues are not specified in this case, so technically anything non-acidic can be used. Possibly, some small polar residue such as Ser or Thr may be a relatively neutral replacement.

RNA may be packaged directly into liposomes, may be alternatively packaged into extracellular vesicles (EVs) such as exosomes and microsomes and may be packaged with or without previous complexion with RNA binding proteins.

A further preferred embodiment of the present vaccine is the provision of the vaccine as vector-based vaccine. These vector-based vaccines have gained new boost in the course of the COVID-19 vaccination and provides also an appropriate strategy for the present vaccination according to the present invention. There are various vectors available for such vaccines (such as adenoviruses, adeno-associated viruses, vesicular stomatitis virus, Newcastle Disease virus, alphaviruses, baculoviruses, retroviruses, such as lentiviruses abd Foamyviruses; especially AdV-26 vectors, modified Vaccinia Ankara vectors, Moloney murine leukemia virus vectors, etc.) as well as various pharmaceutical forms (e.g., freeze dried powder or liquid), routes of administration, formulations, and compositions, thereof which lead to optimized shelf life and storage conditions (recently reviewed e.g. in Crommelin et al., J. Phar. Sci. 110 (2021), 627-634). In such live and/or attenuated vector vaccines a chemically weakened virus is used as vaccine which transports one or more of the P. acnes antigens/epitopes into a subject in order to stimulate an immune response. The P. acnes antigens/epitopes encoding sequences are inserted into the genome of the vector virus, where they are expressed on the viral surface and elicit an immune response in the vaccinated subject.

Based also on these strategies for optimization of mRNA, it is known that N-terminal codons 5-7 as well as the encoded amino acids significantly determine expression strength (Verma et al., Nat. Commun. (2019), 10-5774-s41467_019_Article_13810). Accordingly, this preferred embodiment according to the present invention is not just a matter of selecting optimal codons, but to exchange the sequence in those three positions (i.e. also on protein sequence level), or at least in two of the three positions. This is both an RNA and a protein level effect. Accordingly, preferred derivatives according to the present invention have the following preferred RNA bicodons within those 3 positions 5-7 of specifically: AADUAU (D stands for “not C”), and AAVAUU (V stands for “not U”). This leads to a preferred amino acid sequences K/N-Y and K/N-I, so e.g. KN or KY or KN or KI to occur within residues 5 to 7. On an amino acid level, this results in an amino acid motif starting at protein position 5 or 6, where the motif is K[NY1] and the DNA level sequence is either AADUAU or AAVAUU which increases expression efficiency several fold. For His-tagged polypeptides this is still within the His-tag, for constructs with a signal peptide within the signal peptide, for constructs without either of these this is within the antigen sequence.

The mRNA vaccines according to the present inventions can be formulated according to established methods well available in the art and specifically advanced by the mRNA vaccine applied for SARS-CoV-2 vaccines. Efficient in vivo mRNA delivery is important to achieving therapeutic relevance. There are various characteristics an mRNA vaccine has to fulfil. 1. Safety referring to a non-infectious, non-integrating platform and a possible modification of immunogenicity. 2. Efficacy referring to making the mRNA more stable and highly translatable via carrier molecules. 3. Production referring to an inexpensive and scalable manufacturing.

The type of mRNA carrier and the size of the mRNA-carrier complex have also been shown to modulate the cytokine profile induced by mRNA delivery.

There are two basic approaches for the delivery of mRNA vaccines that have been described to date. First, loading of mRNA into DCs dendritic cells, ex vivo followed by re-infusion of the transfected cells; and second, the direct parenteral injection of mRNA with or without a carrier. Ex vivo DC loading allows precise control of the cellular target, transfection efficiency and other cellular conditions. Direct injection of mRNA does not yet allow precise and efficient cell-type-specific delivery.

Physical delivery methods like for example electroporation or fusion of the mRNA to gold particles is less preferred in comparison to lipid or polymer-based nanoparticles. The cationic peptide protamine has been shown to protect mRNA from degradation by serum RNases as has been used as an immune activator and not as an expression vector. Dendrimers are also cationic lipids and polymers. Besides also small interfering RN (siRNA) were administered. Lipid nanoparticles (LNPs) have become one of the most appealing and commonly used mRNA delivery tools. LNPs often consist of four components: an ionizable cationic lipid, which promotes self-assembly into virus-sized (˜100 nm) particles and allows endosomal release of mRNA to the cytoplasm; lipid-linked polyethylene glycol (PEG), which increases the half-life of formulations; cholesterol, a stabilizing agent; and naturally occurring phospholipids, which support lipid bilayer structure. Systematically delivered mRNA-LNP complexes mainly target the liver owing to binding of apolipoprotein E and subsequent receptor-mediated uptake by hepatocytes. The magnitude and duration of in vivo protein production from mRNA-LNP vaccines can be controlled in part by varying the route of administration. Intramuscular and intradermal delivery of mRNA-LNPs has been shown to result in more persistent protein expression than systemic delivery routes and the sustained antigen availability during vaccination was a driver of high antibody titres and germinal centre (GC) B cell and T follicular helper (T_{FH}) cell responses. Indeed, T_{FH} cells have been identified as a critical population of immune cells that vaccines must activate in order to generate potent and long-lived neutralizing antibody responses.

mRNA can also be associated with a cationic polymer such as polyethylenimine (PEI), with a cationic polymer such as PEI and a lipid component, with a polysaccharide (for example chitosan particle or gel). Furthermore, mRNA can be in a cationic lipid nanoparticle (for example 1,2-dioleoyloxy-3-trimethylammoniumpropane (DOTAP) or dioleoylphosphatidylethanolamine (DOPE) lipids). mRNA can also be complexed with cationic lipids and cholesterol and complexed with cationic lipids, choloestero and PEG lipid.

Adjuvants include novel approaches that take the advantage of intrinsic immunogenicity of mRNA or its ability to encode immunomodulatory proteins. The formulations include cationic nanoemulsions based on the licensed MF59 (Novartis) adjuvant, the TriMix, a combination of mRNAs encoding three immune activator proteins: CD70, CD40 ligand (CD40L) and constitutively active TLR4. The type of mRNA carrier and the size of the mRNA-carrier complex have also been shown to modulate the cytokine profile induced by mRNA delivery. For example, the RNActive (CureVac AC) vaccine platform depends on its carrier to provide adjuvant activity. In this case, the antigen is expressed from a naked, unmodified, sequence-optimized mRNA, while the adjuvant activity is provided by co-delivered RNA complexed with protamine (a poly-cationic peptide), which acts via TLR7 signaling.

According to another aspect, the present invention also relates to a method of production of the polypeptides, vaccines and formulations according to the present invention, wherein the polypeptides comprising at least one P. acnes epitope as defined in these claims are expressed in a host cell, extracted and purified from these host cells, and, optionally, formulated and finished to a pharmaceutical formulation, especially a vaccine for use in the treatment or prevention of P. acnes-associated infections in a human patient.

Therefore, the present invention discloses the following embodiments, specifically drawn i.a to the aspect of the present invention wherein DsA1 an/or DsA2 are used in a product, preferably in a pharmaceutical product, especially in a vaccine either alone or in combination with each other or as DsA1/DsA2 fragments or fusion polypeptides with at least one immunogenic DsA1 epitope and/or at least one immunogenic DsA1 epitope:

1. A vaccine comprising Dermatan sulfate-binding adhesin 1 of P. acnes (DsA1 polypeptide) and/or Dermatan sulfate-binding adhesin 2 of P. acnes (DsA2 polypeptide), and/or a fragment and/or a derivative of DsA1 or DsA2, wherein DsA1 and DsA2 comprise from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Thr repeat containing region (“PT repeat region”), and a C-terminal region (“CTR”), wherein the fragment and/or the derivative comprises or consists at least of a CSD2 fragment, wherein the CSD2 fragment is preferably
a contiguous polypeptide sequence of phenylalanine 150 (F150) to leucine 184 (L184),
a contiguous polypeptide sequence of phenylalanine 150 (F150) to leucine 267 (L267), or
a contiguous polypeptide sequence of histidine 218 (H218) to leucine 267 (L267),
   with the proviso that the fragment or derivative does not comprise an NSR and a CTR, and wherein the amino acid numbering corresponds to the amino acid sequence Q6A5X9 in the UniProt database.
   2. Vaccine according to embodiment 1, wherein the fragment or the derivative comprises or consists at least of
a contiguous polypeptide sequence of phenylalanine 150 (F150) to isoleucine 193 (1193),
a contiguous polypeptide sequence of phenylalanine 150 (F150) to leucine 267 (L267), or
a contiguous polypeptide sequence of histidine 218 (H218) to leucine 267 (L267).
   3. Vaccine according to embodiment 1 or 2, with the proviso that the fragment or the derivative does further not comprise a CSD1 of DsA1 or DsA2, an SR1 of DsA1 or DsA2, an SR2 of DsA1 or DsA2, a CSD3 of DsA1 or DsA2, or a PT repeat region of DsA1 or DsA2, preferably with the proviso that the fragment or derivative does further not comprise a CSD1 of DsA1 or DsA2, an SR1 of DsA1 or DsA2, an SR2 of DsA1 or DsA2, a CSD3 of DsA1 or DsA2, and a PT repeat region of DsA1 or DsA2.
   4. Vaccine according to any one of embodiments 1 to 3, wherein the fragment is
(1) a contiguous polypeptide sequence of phenylalanine 150 (F150) to leucine 184 (L184), further extending one, two, three, or four amino acids at the N-terminus and/or one, two, three, four, five, six, seven, eight, nine, or ten amino acids at the C-terminus;
(2) a contiguous polypeptide sequence of phenylalanine 150 (F150) to leucine 267 (L267), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus;
(3) a contiguous polypeptide sequence of histidine 218 (H218) to leucine 267 (L267) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus;
(4) a contiguous polypeptide sequence of phenylalanine 150 (F150) to leucine 267 (L267), further extending one, two, three, or four amino acids at the N-terminus and/or one, two, three, four, five, six, seven, eight, nine, or ten amino acids at the C-terminus;
(5) a contiguous polypeptide sequence of alanine 148 (F148) to leucine 271 (L271), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus;
(6) a contiguous polypeptide sequence of histidine 146 (H146) to threonine 277 (T277) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus;
(7) a contiguous polypeptide sequence of leucine 238 (L238) to leucine 272 (L272), further extending one, two, three, or four amino acids at the N-terminus and/or one, two, three, four, five, six, seven, eight, nine, or ten amino acids at the C-terminus;
(8) a contiguous polypeptide sequence of leucine 145 (L145) to alanine 201 (A201), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus; and/or
(9) a contiguous polypeptide sequence of leucine 145 (L145) to leucine 280 (L280) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus.
   5. Vaccine according to any one of embodiments 1 to 4, wherein the fragment has a length of at least 35 amino acids, preferably a length of at least 40 amino acids, especially a length of at least 50 amino acids.
   6. Vaccine according to any one of embodiments 1 to 5, which comprises at least one additional polypeptide with an amino acid sequence containing or consisting of at least one additional P. acnes antigen or P. acnes epitope which is not a DsA1 or DsA2 antigen or epitope, preferably at least one additional amino acid sequence containing at least the putative iron-transport protein (PITP) polypeptide of P. acnes or at least one epitope of PITP polypeptide of P. acnes, wherein the PITP polypeptide comprises from N- to C-terminus an extended neocarzinostatin family domain (“ENFD”), a first swapping region (“SR1”), a heme-binding domain (“HbD”), a second swapping region (“SR2”) including the C-terminal LPXT(G) motif, and a hydrophobic C-terminal region (“hLAR”).
   7. Vaccine according to embodiment 6 wherein the derivative comprises at least one additional sequence from the PITP polypeptide, preferably a sequence comprising at least one of ENFD, SR1, HbD, SR2, preferably selected from ENFD and HbD, more preferred a porphyrin-binding domain thereof, especially wherein the PITP polypeptide is a fragment or derivative consisting of the following amino acids: A32 to T430, A32 to G426, A32 to Q198, A32 to T143, A32 to K400, A32 to T159, A32 to I177, A32 to Q204, A32 to G234, A32 to R164, A32 to S391, A32 to P179, A32 to R158, A32 to G147, A32 to E73 and P94 to G147; P34 to T430, P34 to G426, P34 to Q198, P34 to T143, P34 to K400, P34 to T159, P34 to I177, P34 to Q204, P34 to G234, P34 to R164, P34 to S391, P34 to P179, P34 to R158, P34 to G147, P34 to E73 and P94 to G147; S240 to S391, A32 to D441, A32 to I440, A32 to E439, A32 to D438, A32 to S437, A32 to S436, A32 to P435, A32 to G434, A32 to E433, A32 to A432, A32 to G431; P34 to D441, P34 to I440, P34 to E439, P34 to D438, P34 to S437, P34 to S436, P34 to P435, P34 to G434, P34 to E433, P34 to A432, P34 to G431; S240 to D441, S240 to I440, S240 to E439, S240 to D438, S240 to S437, S240 to S436, S240 to P435, S240 to G434, S240 to E433, S240 to A432, S240 to G431; A32 to T430, A32 to V429, A32 to P428, A32 to L427, A32 to G426, A32 to R425, A32 to G424, A32 to A423, A32 to G422, A32 to K421, A32 to G420, A32 to S419, A32 to D418. A32 to D417. A32 to G416, A32 to I415, A32 to V414, A32 to K413, A32 to G412, A32 to T411, A32 to V410, A32 to A409, A32 to D408, A32 to V407, A32 to T406, A32 to V405, A32 to N404, A32 to H403, A32 to C402, A32 to V401, A32 to K400, A32 to E399, A32 to A398, A32 to S397, A32 to L396, A32 to T395, A32 to L394, A32 to N393, A32 to T392; P34 to T430, P34 to V429, P34 to P428, P34 to L427, P34 to G426, P34 to R425, P34 to G424, P34 to A423, P34 to G422, P34 to K421, P34 to G420, P34 to S419, P34 to D418, P34 to D417, P34 to G416, P34 to I415, P34 to V414, P34 to K413, P34 to G412, P34 to T411, P34 to V410, P34 to A409, P34 to D408, P34 to V407, P34 to T406, P34 to V405, P34 to N404, P34 to H403, P34 to C402, P34 to V401, P34 to K400, P34 to E399, P34 to A398, P34 to S397, P34 to L396, P34 to T395, P34 to L394, P34 to N393, P34 to T392; G172 to T430, G172 to V401, G172 to K400, G172 to L396, G172 to N393, A199 to T430, A199 to V401, A199 to K400, A199 to L396, A199 to N393, H223 to T430, H223 to V401, H223 to K400, H223 to L396, H223 to N393, T232 to T430, T232 to V401, T232 to K400, T232 to L396, T232 to N393, G234 to T430, G234 to V401, G234 to K400, G234 to L396. G234 to N393, V238 to T430, V238 to V401, V238 to K400, V238 to L396, V238 to N393, S240 to T430, S240 to V429, S240 to P428, S240 to L427, S240 to 0426, S240 to R425, S240 to G424, S240 to A423, S240 to G422, S240 to K421, S240 to G42G, S240 to S419, S240 to D418, S240 to D417, S240 to G416, S240 to 1415, S240 to V414, S240 to K413, S240 to G412, S240 to T411, S240 to V410, S240 to A409, S240 to D408, S240 to V407, S240 to T406, S240 to V405, S240 to N404, S240 to H403, S240 to C402, S240 to V401, S240 to K400, S240 to E399, S240 to A398, S240 to S397, S240 to L396, S240 to T395, S240 to L394, S240 to N393, S240 to T392; 39I-48I, 40P-49T, 41V-50I, 42G-51S, 43R-52G, 44E-53K, 68P-77N, 69A-78S, 70S-79D, 71V-80K, 72P-81F, 73E-82Y, 74F-830, 75Y-84Y, 760-85D, 77N-86P, 78S-87S, 79D-88K, 80K-89D, 81F-90T, 82Y-91T, 83G-92E, 84Y-93S, 85D-94P, 86P-95S, 87S-96T, 88K-971, 89D-98W, 90T-99V, 91T-100Y, 92E-101T, 93S-102P, 94P-103S, 95S-104Q, 96T-105K, 971-106A, 98W-107I, 99V-108G, 100Y-109S, 101T-110R, 102P-111F, 103S-112A, 104Q-113Q, 105K-1140, 106A-115R, 1071-116P, 1080-117M, 109S-118N, 110R-119N, 111F-120D, 112A-121G, 125I-134Q, 126T-135G, 127M-136K, 128K-137D, 144K-153H, 145A-154S, 146H-155D, 147G-156D, 148V-157T, 149G-158R, 150K-159T, 151T-160P, 152D-161V, 153H-162T, 154S-163Y, 155D-164R, 156D-165E, 157T-166A, 158R-167T, 159T-168P, 160P-169A, 161V-170P, 162T-171T, 163Y-172G, 164R-173P, 165E-174K, 166A-175T, 167T-176P, 168P-177I, 169A-178A, 170P-179P, 171T-180S, 172G-181K, 173P-182Q, 174K-183P, 175T-184S, 176P-185K, 177I-186Q, 178A-187A, 179P-188A, 180S-189P, 181K-190S, 182Q-191K, 183P-192Q, 184S-193V, 185K-194K, 186Q-195P, 187A-196S, 188A-197K, 189P-198Q, 190S-199A, 191K-200G, 192Q-201P, 193V-202N, 194K-203K, 195P-204Q, 196S-205S, 197K-206T, 198Q-207T, 199A-208P, 200G-209Q, 201P-210Q, 202N-211K, 203K-212T, 204Q-213A, 205S-214E, 206T-215H, 207T-216R, 208P-217S, 209Q-218Q, 210Q-219T, 211K-220P, 212T-221A, 213A-222A, 214E-223H, 215H-224R, 216R-225T, 217S-226M, 218Q-227T, 219T-228K, 220P-229Q, 221 A-230V, 222A-231C, 223H-232T, 224R-233I, 225T-234G, 226M-235A, 227T-236S, 228K-237K, 229Q-238V, 230V-239T, 231C-240S, 232T-241G, 233I-242S, 266L-275S, 267S-276A, 268G-277F, 282T-291K, 334S-343N, 353G-3621, 354V-363K, 355S-364G, 356V-365S, 357S-366P, 358G-367V, 359N-368K, 377F-386P, 378A-387M, 379G-388N, 380F-389P, 396L-405V, 397S-406T, 398A-407V, 399E-408D, 400K-409A, 401V-410V, 402C-41 IT, 403H-412G, 404N-413K, 405V-414V, 406T-4151, 407V-416G, 408D-417D, 409A-418D, 410V-419S, 411T-420G, 412G-421K, 413K-422G, 414V-423A, 415I-424G, 416G-425R, 417D-426G, 418D-427L, 419S-428P, 420G-429V, 421K-430T, 422G-431G, 423A-432A, 424G-433E, 425R-434G, 426G-435P, 427L-436S, 428P-437S, 429V-438D, 430T-439E, 431G-440I, 432A-441D, 433E-442L, 434G-443G, 435P-444I, 436S-445V, and 437S-446G; I39-K53, P68-G121, I125-D137, K144-S242, L266-F277, T282-K291, S334-N343, G353-K368. F377-P389, L396-G446, A32 to R164, A32 to Q198, A32 to T143, A32 to V148, A32 to T171, P34 to R164, A32 to T159, A32 to I177, A32 to Q204, A32 to G234, A32 to K400, A32 to S391, V238 to K400, A199 to T430, V238 to T395, G234 to K400, H223 to K400, T232 to V401. V238 to T392, V238 to N393, V238 to L394. V238 to T395, V238 to L396, T232 to T430, G172 to K400, and G172 to G234, and fragments thereof comprising at least of a PITP epitope.
   8. Vaccine according to any one of embodiments 1 to 7 comprising a derivative wherein the derivative comprises at least one fragment of DsA1 and at least one fragment of DsA2.
   9. Vaccine according to any one of embodiments 1 to 8, wherein in the DsA1/DsA2 fragment or derivative at least 5 PT repeats, preferably at least 10 PT repeats, especially at least 15 PT repeats, are deleted compared to a naturally occurring wild type DsA1/DsA2 polypeptide (“native DsA1/DsA2”), and wherein preferably at least one, more preferred at least two, more preferred at least three, even more preferred at least four, especially five, PT repeat(s) is/are present.
   10. Vaccine according to any one of embodiments 1 to 9, wherein the derivative further comprises an NSR, CSD1, SR1, CSD2, SR2, CSD3 and/or CTR from at least one other DsA1 and/or DsA2 or from at least one other P. acnes strain than the strain from which the CSD2 fragment.
   11. Vaccine according to any one of embodiments 1 to 10, wherein the fragment or derivative lacks the NSR, and/or the CTR.
   12. Vaccine according to any one of embodiments 1 to 11 further comprising another P. acnes antigen or epitope, preferably an antigen selected from DsA2 and PITP and/or a DsA2 epitope and/or an PITP epitope.
   13. Vaccine according to any one of embodiments 1 to 12 further comprising an epitope containing fragment of another P. acnes polypeptide, preferably an epitope containing fragment of DsA2 and PITP.
   14. Vaccine according to any one of embodiments 1 to 13, wherein the vaccine is protective against development of a pathological condition induced by P. acnes invasion.
   15. Vaccine according to any one of embodiments 1 to 14, wherein the vaccine is formulated for administration to a human subject, e.g. a child, an adolescent or adult subject.
   16. Vaccine according to any one of embodiments 1 to 15, wherein the vaccine is formulated for use in a treatment regimen involving both, a prime and boost immunization, preferably wherein the same formulation is used for the prime and boost administration.
   17. Vaccine according to any one of embodiments 1 to 16, wherein the vaccine is formulated with an adjuvant, preferably with an adjuvant selected from the group consisting of mineral salts, preferably alum (potassium aluminum sulfate), aluminum hydroxide, aluminum phosphate, aluminum oxohydroxide, aluminum hydroxyphosphate, calcium phosphate, cerium nitrate, zinc sulfate, colloidal iron hydroxide, and/or calcium chloride; oil-in-water emulsions, liposomes, TLR agonists, Monophosphoryl Lipid A, saponins, phospholipids, emulsion-based adjuvants, aluminium hydroxide gel, solid phase adsorbents, nanospheres and encapsulating materials such as liposomes; high molecular weight adjuvants, preferably toxins, toxoids or any mutant cross-reactive material of the toxin from tetanus, diphtheria, pertussis. Pseudomonas species, E. coli, Staphylococcus species, and Streptococcus species. Such toxins or toxoids may be tetanus toxoid, pertussis toxoid, cholera toxoid, E. coli LT, E. coli ST, and exotoxin A from Pseudomonas aeruginosa; bacterial outer membrane proteins such as outer membrane protein complex c (OMPC), porins, transferrin binding proteins, pneumolysin, pneumococcal surface protein A (PspA), pneumococcal adhesin protein (PsaA), C. difficile enterotoxin (toxin A) and cytotoxin (toxin B) or Haemophilus influenzae protein D, other pharmaceutically acceptable polypeptide carriers, such as ovalbumin, keyhole limpet hemocyanin (KLH), bovine serum albumin (BSA) or purified protein derivative of tuberculin (PPD); cationic peptides, CpG oligonucleotide, squalene-based adjuvants, preferably MF59: cytokines, such as IL-1 and IL-2; or formulated as virus-like particles or nanoparticles; or formulated as virus-like particles or nanoparticles; or combinations thereof.
   18. Vaccine according to any one of embodiments 1 to 17, wherein the vaccine is formulated for intradermal, subcutaneous (s.c.), parenteral, preferably intramuscular (i.m.), mucosal, transcutaneous or topical administration.
   19. Vaccine according to any one of embodiments 1 to 18, wherein the vaccine comprises 0.1 μg to 5 mg, preferably 0.5 to 1000 μg, more preferred 1 to 500 μg, even more preferred 5 to 300 μg, especially 10 to 200 μg, each of the DsA1 and/or DsA2 and/or PITP, and/or a fragment or derivative of DsA1 or DsA2 or PITP per dose.
   20. Vaccine according to any one of embodiments 1 to 19, wherein the DsA1 and/or DsA2 and/or PITP, and/or a fragment or derivative of DsA1 or DsA2 or PITP is provided as DNA or RNA encoding an antigen or an epitope containing fragment or derivative thereof, preferably as mRNA vaccine, especially an mRNA vaccine formulated with cationic polymers, with an mRNA molecule with the following structure: 5′UTR-signal peptide-encoded antigen or epitope-3′UTR; or as a vector-based vaccine, especially with an adenovirus vector, an adeno-associated virus vector, a vesicular stomatitis virus vector, a Newcastle Disease virus vector, an alphavirus vector, a baculovirus vector, or a retrovirus vector; and/or wherein the coding sequence on DNA level has the initial coding DNA sequence ATGGTG.
   21. Vaccine according to any one of embodiments 1 to 20, wherein the vaccine comprises 5 to 500 μg each, preferably 20 to 100 μg each, of DsA1 and/or DsA2 and/or PITP, and/or a fragment or derivative of DsA1 or DsA2 or PITP; or DNA or RNA encoding an antigen, or a fragment or a derivative thereof, per dose.
   22. Vaccine according to any one of embodiments 1 to 20, wherein the vaccine comprises less than 1 μg each of DsA1 and/or DsA2 and/or PITP, and/or a fragment or derivative of DsA1 or DsA2 or PITP per dose.
   23. Vaccine according to any one of embodiments 1 to 22, wherein the vaccine does not contain Freund's incomplete adjuvant.
   24. Vaccine according to any one of embodiments 1 to 23, wherein the vaccine contains a composite immunogen and is engineered by linking of one or more antigen(s) of P. acnes and/or one or more cross-reactive, especially cross-type-reactive, or otherwise immunologically relevant epitope(s) of P. acnes to each other, preferably wherein the immunogen comprises or consists of at least 2 (e.g. 2, 3, 4, 5, 6, 7, 8, 9, or 10) antigen(s) and/or immunologically relevant epitope(s) thereof, especially wherein the antigens and/or epitopes are cross-reactive, especially cross-type-reactive.
   25. Vaccine according to any one of embodiments 1 to 24, wherein the vaccine contains a composite immunogen comprising or consisting of at least two (e.g., 2, 3, 4, 5, 6 7, 8, 9, or 10) antigen(s) of P. acnes and/or epitope(s) thereof linked to an adjuvant, wherein at least one antigen or epitope is immunologically relevant.
   26. Vaccine according to any one of embodiments 1 to 25, wherein the vaccine contains a composite immunogen comprising at least two antigen(s) and/or epitopes thereof of P. acnes covalently linked to each other, preferably wherein the immunogen comprises at least two antigen(s) and/or epitopes thereof further linked to an adjuvant, especially wherein at least one antigen or epitope is immunologically relevant.
   27. Vaccine according to any one of embodiments 1 to 26, wherein the vaccine contains a composite immunogen wherein one antigen or epitope thereof is linked to an adjuvant, especially wherein the at least one antigen or epitope is immunologically relevant.
   28. Vaccine according to any one of embodiments 1 to 27, wherein the vaccine comprises a variant of an antigen or of an epitope.
   29. Vaccine according to any one of embodiments 1 to 28, wherein the vaccine comprises a polypeptide with at least about 90% amino acid sequence identity, preferably at least 95%, more preferably at least 98%, more preferably at least 99% or at least 99.5%, to the amino acid sequence of a naturally occurring P. acnes antigen; or a polypeptide of a P. acnes epitope, wherein at least 1, 2, 3, 4, up to 5 point mutations are contained in the amino acid sequence compared to the naturally occurring epitope.
   30. Vaccine according to any one of embodiments 1 to 29, wherein the vaccine comprises a variant, preferably a DsA1 and/or DsA2 protein wherein one or more amino acid residues are added, or deleted, at the N- or C-terminus, as well as within one or more internal domains, especially a variant comprising additional amino acids at the N-terminal and/or at the C-terminal end, to prolong an antigen sequence, e.g. to prolong a sequence of an epitope or epitopic region within a protein by at least one amino acid residue, preferably by less than 3 amino acids, specifically less than 5, or else less than 10 amino acids.
   31. Vaccine according to any one of embodiments 1 to 30, wherein the vaccine comprises a variant as fusion protein, wherein a DsA1 and/or DsA2 antigen sequence is prolonged by additional amino acid residues of another polypeptide or protein, preferably by one or more immunologically relevant epitopes.
   32. Vaccine according to any one of embodiments 1 to 31, wherein the vaccine comprises a fragment or derivative of DsA1 or DsA2, wherein DsA1 and DsA2 comprise from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Leu repeat containing region (“PT repeat region”), and a C-terminal region (“CTR”), wherein fragment or derivative comprises or consists at least of (1) a contiguous polypeptide sequence of phenylalanine 150 (F150) to leucine 184 (L184), (2) a contiguous polypeptide sequence of phenylalanine 150 (F150) to leucine 267 (L267), or (3) a contiguous polypeptide sequence of histidine 218 (H218) to leucine 267 (L267), with the proviso that the fragment or derivative does not comprise the NSR and the CTR, said fragment or derivative optionally comprising one or more amino acid exchanges, wherein one or more amino acid residues are changed to a cysteine (Cys, C) residue to further stabilise the fragment or derivative by the ability to form disulfide bonds.
   33. Vaccine according to any one of embodiments 1 to 32, wherein the fragment or derivative has a length of at least 20, preferably at least 30, more preferred at least 60 amino acids, even more preferred a length of at least 80 amino acids, especially a length of at least 100 amino acids.
   34. Vaccine according to any one of embodiments 1 to 33, wherein the fragment or derivative has a length of less than 400 amino acids, preferably less than 350 amino acids, especially less than 300 amino acid residues.
   35. Vaccine according to any one of embodiments 1 to 33, wherein the fragment or derivative has a length of less than 250 amino acids, preferably less than 200 amino acids, especially less than 150 amino acid residues.
   36. Vaccine according to any one of embodiments 1 to 35, wherein two or more amino acid residues in a fragment or derivative are changed to a cysteine (Cys, C) residue to further stabilise the fragment or derivative by the ability to form disulfide bonds, preferably (with respect to the DsA1 sequence (which is also applicable to the DsA2 sequence; FIG. 12A)) containing at least one of the amino acid pairs in the following group wherein the native amino acid is exchanged to cysteine: PHE150-GLY185, LEU167-ALA199, ASN157-ALA192, LYS166-ALA199, ALA153-GLY 185, ASP164-ALA199, VAL154-ALA179, PHE150-LEU184, VAL154-ALA188, ALA153-ALA188, ALA179-GLU191, THR178-GLU191, ALA161-ALA195, ILE175-ALA195, ILE175-GLU191, LEU158-ALA176, ASN157-ALA188, ASN157-ALA179, ASP164-GLU196, VAL154-LEU180, ALA153-LYS189, ASN157-LYS189, ALA161-ALA192, LEU167-ALA198, LEU158-ALA179, ALA160-ALA192, LYS165-ALA199, LYS166-GLN200; VAL205-ALA260, ILE 232-ARG266, SER235-ILE263, SER235-ARG262, ILE232-LEU267, PRO236-ILE263, ALA231-ARG266, ASN239-ILE259, ILE232-ILE263, VAL209-PRO236, ALA201-ALA256, SER235-ILE259, VAL228-ARG266, ALA202-ALA256, ALA201-VAL257, GLY206-VAL243, GLY208-ILE264, GLY208-ILE263, VAL205-ASN239, LEU238-ILE259, GLY208-ALA260, ALA201-LYS253, ALA202-VAL243, LEU204-ALA260, VAL205-ALA256, VAL205-ILE259, SER235-ARG266, ASN239-ALA260, VAL209-ILE263, or VAL209-ASN239.
   37. Vaccine according to any one of embodiments 1 to 36, wherein a cysteine in the DsA1, DsA2 or PITP or fragment or derivative thereof is exchanged, preferably wherein the cysteine is exchanged with serine, proline, alanine, threonine, asparagine, glutamine, valine, isoleucine, leucine, phenylalanine, tyrosine and glycine, more preferred with serine, proline, alanine, threonine, asparagine, glutamine, valine, isoleucine, leucine, especially with serine, proline, alanine, threonine, asparagine, and glutamine.
   38. Vaccine according to any one of embodiments 1 to 38, wherein the fragment or derivative has a length of length of 35 to 350 amino acids, preferably of 40 to 300 amino acids, more preferred of 50 to 250 amino acid residues, more preferred of 60 to 200 amino acids, more preferred of 70 to 150 amino acids, especially of 90 to 130 amino acid residues.
   39. Vaccine according to any one of embodiments 1 to 37, wherein the derivative comprises at least one polypeptide stretch of DsA1 and at least one polypeptide stretch of DsA2, preferably wherein the derivative comprises at least CSD1, CSD2 or CSD3 of DsA1 and at least CSD1. CSD2 or CSD3 of DsA2.
   40. Vaccine according to any one of embodiments 1 to 39, wherein the derivative comprises at least one fragment comprising at least CSD1, CSD2 or CSD3 of DsA1 and at least one fragment comprising at least CSD1, CSD2 or CSD3 of DsA2 and wherein the at least two fragments are interconnected by
an SR1 or SR2 of DsA1 or DsA2 adjacent to the at least CSD1, CSD2 or CSD3 of DsA1 and at least CSD1, CSD2 or CSD3 of DsA2: or
an amino acid sequence consisting of an N-terminal part of an SR1 or SR2 of DsA1 or DsA2, adjacent to the at least CSD1, CSD2 or CSD3 of DsA1 and at least CSD1, CSD2 or CSD3 of DsA2, and a C-terminal part of a different SR1 or SR2 of DsA1 or DsA2, adjacent to the at least CSD1, CSD2 or CSD3 of DsA1 and at least CSD1, CSD2 or CSD3 of DsA2, wherein the total number of amino acid residues the amino acid sequence consisting of the of the N- and C-terminal part is identical to the SR1 or SR2 of DsA1 or DsA2.
   41. Vaccine according to any one of embodiments 1 to 40, wherein the derivative comprises at least one fragment comprising at least CSD1, CSD2 or CSD3 of DsA1 and at least one fragment comprising at least CSD1, CSD2 or CSD3 of DsA2, wherein the fragment of DsA1 and DsA2 has independently a length of at least 20 amino acid residues, preferably at least 30, more preferred at least 40, especially at least 50, amino acid residues
   42. DsA1 and/or DsA2, and/or a fragment and/or a derivative of DsA1 or DsA2 according to any one of embodiments 1 to 41 for use in a therapeutic and prophylactic treatment, preferably for use in the treatment or prevention of P. acnes-associated infections, especially selected from the group consisting of acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, artherosclerosis, prostethic cancer and a medical implant biofilm infection by P. acnes.
   43. DsA1 and/or DsA2, and/or a fragment and/or a derivative of DsA1 or DsA2 according to any one of embodiments 1 to 41 for use in the treatment or prevention of P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes.
   44. DsA1 and/or DsA2, and/or a fragment and/or a derivative of DsA1 or DsA2 for use according to embodiment 42 or 43, wherein the human patient is suffering from P. acnes-associated infections and pathological conditions associated with at least two P. acnes MLST phylotypes, and/or at least two different CC types of P. acnes and/or at least two ribotypes of P. acnes, as determined by a surface binding measurement employing flow cytometry and in a bactericidal killing assay using immune serum raised against such vaccine antigen.
   45. DsA1 and/or DsA2, and/or a fragment and/or a derivative of DsA1 or DsA2 for use according to any one of embodiments 42 to 44, wherein the human patient is suffering from P. acnes-associated infections and pathological conditions associated with at least two or three genetically different types of P. acnes strains, preferably against at least one Type I and at least one Type II or III strains; or from at least one Type II and at least one Type I or III strains; or from at least one Type III and at least one Type I or II strains; or from at least one Type I, at least one Type II, and at least one Type III strain, especially Types IA1, IA2, and II.
   46. DsA1 and/or DsA2, and/or a fragment and/or a derivative of DsA1 or DsA2 for use according to any one of embodiments 42 to 45, wherein the human patient is suffering from P. acnes-associated infections and pathological conditions associated with two or more of P. acnes strains selected from the group consisting of Type IAI1, Type IA2, Type IB, Type IC, Type II and Type fit strains, or Type I-Ia, I-Ib, I-2 and II, as defined according to the MLST typing schemes, especially Types IA1, IA2, and II.
   47. DsA1 and/or DsA2, and/or a fragment and/or a derivative of DsA1 or DsA2 for use according to any one of embodiments 42 to 46, wherein the human patient is suffering from P. acnes-associated infections and pathological conditions associated with two or more of P. acnes strains selected from the group of the ribotypes as determined according to 16S ribosomal sequence differences.
   48. DsA1 and/or DsA2, and/or a fragment and/or a derivative of DsA1 or DsA2 for use according to any one of embodiments 42 to 47, wherein the human patient is suffering from P. acnes-associated infections and pathological conditions associated with two or more phylotypes IA1, IA2, IB1, IB2, IB3, IC, II and III determined by the analysis of a single locus (SLST), especially Types IA1, IA2, and II.
   49. DsA1 and/or DsA2, and/or a fragment and/or a derivative of DsA1 or DsA2 for use according to any one of embodiments 42 to 48, wherein the human patient is suffering from P. acnes-associated infections and pathological conditions associated with at least two different phylogenetic types, more preferably three, even more preferably four, five or six phylogenetic types of the following group of strains: NCTC737, KPA171202 (DSMZ, German Collection of Microorganisms and Cell Cultures. Braunschweig, Germany), SK137, HL005PA1, HL005PA4, HL013PA1, HL030PAL, HL043PA1, HL053PAL, HL053PA2, HL050PA1, HL050PA2, HL060PA1, HL110PA4 (BE, Biodefense and Emerging Infections Research Resources Repository, Manassas, VA), P.acn31 and Asn12 (McDowell et al. 2012) and IAI 008, IAI031, IAI034, IAI035, IAI038, IAI040, IAI042, IAI045, IAI041 (Charite Berlin, Pro-Implant foundation).
   50. DsA1 and/or DsA2, and/or a fragment and/or a derivative of DsA1 or DsA2 for use according to any one of embodiments 42 to 49 for use in protection against infection with P. acnes.
   51. DsA1 and/or DsA2, and/or a fragment and/or a derivative of DsA1 or DsA2 for use according to any one of embodiments 42 to 50 for use in prevention or reducing development of a P. acnes-associated indication.
   52. DsA1 and/or DsA2, and/or a fragment and/or a derivative of DsA1 or DsA2 for use according to any one of embodiments 42 to 51, wherein the vaccine repeatedly administered to a human patient, preferably by at least 2 or 3 administrations, or at least 4, 5, or even more repeated administrations.
   53. DsA1 and/or DsA2, and/or a fragment and/or a derivative of DsA1 or DsA2 for use according to any one of embodiments 42 to 52, wherein the vaccine is used in one or more treatment cycles, each involving at least two, or three or four consecutive administrations within a period of 1 year in intervals of at least 1 or 2 weeks, especially wherein the treatment cycles are repeated at least any of 1×, 2×, 3×, or 4×, even more times, e.g. within a period of 5 years.
   54. DsA1 and/or DsA2, and/or a fragment and/or a derivative of DsA1 or DsA2 for use according to any one of embodiments 42 to 53, wherein the vaccine provokes an immune response, including the production of antibodies and/or cytokines and/or the activation of cytotoxic T cells, B-cells, antigen presenting cells, helper T cells, dendritic cells and/or other cellular responses in an individual upon administration.
   55. DsA1 and/or DsA2, and/or a fragment and/or a derivative of DsA1 or DsA2 for use according to any one of embodiments 42 to 54, wherein the vaccine is administered to a patient with a skin or other affected organ or tissue condition in which the strains from any of Type I, II, or III P. acnes, or a combination of at least two phylotypes of Type I, 1I, and III, or of at least two ribotypes of P. acnes are involved as pathogenic factors, or to a healthy individual that may be susceptible to an infection by any of the P. acnes strains, especially Type IA1, IA2, and II.
   56. DsA1 and/or DsA2, and/or a fragment and/or a derivative of DsA1 or DsA2 for use according to any one of embodiments 42 to 55, wherein the vaccine is administered to a patient by intradermal, transdermal or a subcutaneous administration, preferably by intradermal injection applicators, microneedles, transdermal laser devices, topical, skin patch or other suitable skin-adapted application device.
   57. DsA1 and/or DsA2, and/or a fragment and/or a derivative of DsA1 or DsA2 for use according to any one of embodiments 42 to 55, wherein the vaccine is administered by oral or parenteral administration such as intramuscular, intranodal, intranasal, mucosal, intrapulmonary, vaginal, rectal or other suitable routes.
   58. Fragment or derivative of DsA1, DsA2 or PITP as defined in any one of embodiments 1 to 57, preferably a fragment or derivative according to SEQ ID NOs: 5 to 7, 9 to 0.12, 14 to 20, and 29 to 50.
   59. Pharmaceutical composition comprising a fragment or derivative according to embodiment 58, or DsA1 or DsA2.
   60. Pharmaceutical composition according to embodiment 59, further comprising a pharmaceutically acceptable carrier.
   61. A method for producing a vaccine, wherein DsA1 and/or DsA2, and/or a fragment and/or a derivative of DsA1 or DsA2 as defined in any one embodiments 1 to 41 is mixed with a pharmaceutically acceptable excipient or carrier and, optionally, finished into a pharmaceutically administrable vaccine dose, preferably in a dosage unit suitable for administration to an individual, especially a human individual.
   62. Vaccine or P. acnes polypeptide, comprising
   (a) a CSD2 fragment of DsA1 or DsA2 within A148 to L267 of DsA1 or A192 to L311 of DsA2, at least comprising a contiguous polypeptide sequence of 20 amino acid residues, especially F150 to L184 of DsA1 or F194 to L228 of DsA2,
   (b) a CSD2 derivative of (a) with one or more, preferably one, two or three, especially one, of the following amino acid exchanges: A149T, D152N, D152S, V1551, V163I, K166H, K166P, A168T, K169R, T171A, V173M, A176V, A183T, T190F, T190I, E191K, A192L, A192F, A198G, A199T, A202T, V205I, G206N, G206S, G206R, G206Q, K212Q, A214T, I219V, I219A, S223A, D225N, V233I, S235F, N239S, L255I, V2571, Q258S, I259L, R262H, I264V, D265K in DsA1 and T193A, N196D, N196S, I199V, I207V, H210K, H210P, A212T, R213K, T215A, V217M, V220A, T227A, F234T, F234I, E235K, L236A, L236F, A242G, A243T, A246T, I249V, R250G, R250S, R250N, G250Q, Q256K, A258T, V2631, V263A, A267S, D269N, I277V, S279F, S283N, I299L, I301V, S302Q, L303I, H306R, V308I, K309D in DsA2,
   (c) a CSD1 fragment of DsA1 or DsA2 within 149 to L130 of DsA1 or I93 to L174 of DsA2, at least comprising a contiguous polypeptide sequence of 20 amino acid residues, especially T117 to A127 of DsA1 or T161 to LA171 of DsA2,
   (d) a CSD1 derivative of (c) with one or more, preferably one, two or three, especially one, of the following amino acid exchanges: D50N, D50S, K51E, K51A, C53Y, D55K, V57I, A61L, A65G, A68V, L71M, F76L, S78A, V82M, V82A, L84M, P86R, P86S, K90R, K90A, K94A, V97T, L99T, I100L, K104R, K106E, A107V, I109V, G110A, A111S, L113V, G114S, L116V, T117A, K120A, I121V, R123H, A124T, V1281 in DsA1 and S94D, S94N, E95A, E95K, C97Y, K99D, I101V, 105A, G109A, V112A, L115M, L120F, A122S, A126M, A126V, L128M, P130R, P130S, A134K, A134R, A138K, T141V, T143L, 1144L, R148K, K150E, V151A, V153I, A154G, S155A, L157V, G158S, V160L, A161T, A164K, V165I, H167R, A168T, I172V in DsA2
   (e) a CSD3 fragment of DsA1 or DsA2 within A278 to K323 of DsA1 or A322 to E366 of DsA2, at least comprising a contiguous polypeptide sequence of R266 to T277 or R286 to K296 of DsA1 or V333 to Q347 of DsA2
   (f) a CSD3 derivative with one or more, preferably one, two or three, especially one, of the following amino acid exchanges:
   M281V, N283D, T285A, R286Q, V289A, V291I, I292R, T293N, A294T, D295K, D295Q, K296E, I298V, K299A, T300V, T300I, A301Y,—between 301 and 302 D, E302K, E305K, K306A, A310T, D313G, K316Q, S320T, C321—, P322L, K323D, K323E in DsA1 and V325M, D327N, A329T, R330Q, V333A, I335V, R3361, N337T, T338A, Q339K, Q339D, E340K, I342V, A343K, V344T, V344I, Y345A, gap between 345 and 346 D, K346E, K349E, A350K, T354A, G357D, Q360K, T364S, gap between 364 and 365 C, P365L, E366D, E366K, in DsA2.
   63. Vaccine, composition, method or DsA1 and/or DsA2 derivative according to any one of embodiments 1 to 62 wherein the DsA1 and/or DsA2 derivative comprises one or more, preferably one, two or three, especially one, of the following amino acid exchanges:
   DsA1: A301Y, G206L, G206F, A192L, G206R, G206W, G206Q, G206K, A301F, A301L, G206I, A301W, A301I, G206E, G206M, G208L, G206V, A192M, A256L, A192I, A68I, G206N, A68V, I259L, A161L, V205I, G206T, G206H, A68L, 0206Y, A2561, G206A, 68M, A192F, G110A, N239L, G206C, A301V, E305L, A93I, A93L, S223A, E305I, E305V, E305A, K120V, K120L, N157I, K120C, E305C, E305M, N239I, K120I, R248C, N239V, E191I, E305F, S78I, K120M, E191L, R248L, H146V, E191V, H146I, S78L, S78V, N157V, H146C, K120F, R248I, R248M, D164L, P236V, P236L, H146L, H146F, D164I, H146M, P86L, R248G, N239C, N157C, E305T, E191C, S78C, R248F, G221L, E305G, P236F, T171L, P86V, R248S, N157L, K120A, H146A, P861, N157F, S235A, R248A, E191M, N157M, A124I, P236A, S78F, P236C, S78M, A161I, N239M, T171I, E191F, S235V, P86F, H146G, S223I, E305S, D164F, E305Y, P236M, S223L, N239F, H146Y, P236I, G221V, D164C, K120G, D164M, S235I, R248V, A124V, S235L, S223V, S147L, S235C, P86M, G208I, D164A, S147I, N239A, K120T, T117I, G110L, R248Y, P182I, A198L, A124L, A215L, A127L, A66L, P86C, G221F, R248T, T171F, P182V, S147V, P271V, T171V, G110V, P271A, E191A, A149L, K120Y, G114I, K120S, S235F, A201I, G131V, A201V, R248N, A66V, A202I, G221C, G114V, A229L, H146T, A124F, A127F, T117V, S223C, A215F, A127I, P182C, A215V, G162L, G162I, G208F, A201L, A161F, N157A, A198V, E191Y, A161V, S223F, G221M, R248W, G131C, A202V, N157T, S147F, N157Y, G114A, T171C, H146W, S78T, A149I, G250L, N239T, A149V, G208C, K120P, G250V, S235G, S223M, D164V, N239G, T171M. E191G, S78A, D164Y, P271I, G110F, G110C, D164W, P236Y, A124C, A66F, S147M, G75L, E305N, S147C, G221I, A127M, P182L, G221A, G110M, A66C, E305Q, P271F, A215M, P236W, K120W, H146P, A161C, A229I, S78G, G114M, P86A, E305W, V154L, G185V, G75I, S223Y, G75V, R248Q, T277V, E191S, A229F, T277G, G131I, N239S, P271C, V154I, A66M, D164G, G208A, P236T, R248D, D164T, A127C, A215I, A161M, P86W, A201F, C53L, T117F, G131A, T117C, A318L, F150L, G208M, P271M, A149C, G208V, G114C, G162F, S223W, A198I, N157G, A202L, P236G, G185I, A198F, A149M, P86Y, E191W, N157W, I263L, S235T, E305P, E305R, A201C, A201M, F150I, G96I, G114F, G221Y, A199L, D164S, A124M, A179I, A202F, H146S, A231L, C53I, I263V, G162M, K120Q, N239Y, G96L, G75C, N157S, T277C, T117A, A199V, G162C, P271Y, G250C, A127Y, A66I, T117L, G185C, A229C, R248P, A1441, T117M, P86T, A260L, G185A, E191Q, G131L, T277I, G250I, G131M, T277A, A127V, F150V, A318I, S78W, S147Y, A198M, P860, A215C, A70I, S235M, A179V, A202M, A144V, A202C, G208W, T171Y, S147W, G208Y, G250F, A195L, K120H, A127W, A66Y, S223G, K120N, S78Y, E191N, A198C, P271G, V154F, A70V, H146N, A229M, A134L, P86S, S78Q, A199I, G75M, A68C, E191T, A89L, G96A, D164P, C53V, S78P, G185L, A149G, A149F, P182A;
   DsA2: A161L, A161I, A126I, A126V, A126L, S175G, G190H, A161T, S183T, R336I, A126F, A161F, A329L, A161V, S283L, A205L, G109A, A329I, G265F, A161W, A232I, S175W, R336L, A300I, K349I, A161M, A300L, G109L, G109I, A246L, S183M, A223L, E235V, G252L, A329M, A126W, S183L, A259L, A329V, A329F, S183I, A242L, G109F, R336V, K117I, R336C, K117C, K349V, R336A, R336F, K117L, R336M, E235I, R310A, K349L, K117V, R310V, K349C, N201I, R310I, R310C, K349F, K349M, K117M, R292C, N201V, R336G, R292L, K117F, E235C, P130L, E235A, D208L, P280L, E235L, R292M, R336T, P280V, R292I, N201L, E235M, P130I, K117A, K349A, P130V, R292G, R310F, R336S, R336Y, N201C, K117G, R310L, R310M, R3100, R336W, D208V, R292F, P130F, K349Y, K117T, R310T, P280C, R292S, N201F, R310S, T215L, N201M, S279C, R292A, S279L, P280F, D208C, S175V, A126M, P280A, S279A, P130C, S279V, E235F, P186C, P130M, K349T, A126C, K349G, N320V, K117S, N320I, S283V, A168I, N320L, S175I, E235Y, P186V, P280I, P186A, P280M, G265L, D208M, P186L, K117Y, S283I, A205I, D208F, A168L, T215I, R292V, A133L, R336P, R310Y, A168V, S279F, S183V, A164I, S279I, S175L, D208A, K349S, E235T, S175C, A122I, D208I, A126Y, A123L, K117P, N320C, R292T, S283C, E235W, G252I, T215V, R292Y, R336Q, S175A, A123I, E235G, A205V, A171V, A133V, A245I, S183C, P226V, R310P, G1811, P226I, A168C, A246I, A171L, N201A, S175M, G158I, A164L, T215C, A164C, P280W, A133C, G119I, T215F, A245V, N320M, A122V, A123V, G206L, N320A, G265V, A122L, A193L, S191V, R310W, G252F, G181V, E235S, R292N, P130W, G119V, G265C, S279M, A259V, A259F, A168F, A168M, A246V, P226C, R292W, G158V, P130Y, G206V, P186G, C97L, N201Y, A171C, G265M, G140L, K117N, G140I, D208G, A193V, G181F, K349W, S279G, A245L, A205F, G158A, A193I, K117W, A122C, R310D, G294V, P186T, S175F, S191C, N201T, K349Q, R310N, A242V, T215M, D208Y, T321C, G252C, I276L, A259M, A171F, N201G, G119F, D208T, P186M, G229V, G294L, S191I, P186S, G119C, G2291, P130A, A205C, A171M, T321V, A193C, F268L, A133M, A123F, G357A, G109V, K117Q, N320G, R310Q, T321G, S283M, P186Y, G229A, R336D, N201S, S183F, F194L, F268V, A245F, G265A, 0252A, R336H, A259I, A133I, A123C, P130T, G113L, D208S, P280T, F268I, G181C, P280Y, R336E, P280G, G158C, N320Y, G140V, K349P, R292D, D208W, N320F, G265I, R336N, A164M, G206M, F268C, G158M, R292P, S191L, G158F, G252M, A245C, G229C, E235Q, G252V, A205M, G206F, R292Q, G181M, A126G, A243L, K349N, S283A, A246M, S279T, P186I, A246F, A246C, A223I, C97I, A164V, A245M, A171I, G357I, A126T, S283F, A123M, F194I, K117D, P226L, P130S, G140F, I276F, F194V, A243V, P130G, G181L, G229F, A259C, G113I, K117H, N320S, G119M, A239L, G206C, A114I, E235N, K349D, A242I, A161C, G294C, G119L. A126S, T321L, A193M, A223V, A122M, G113C, A242C, N201W, R292H, A275L, A242M, G357V, P186F, G294I, D208P, A259W, A243I, R310E, G140C, A242F, G265W, S183A, G109C, G113F, S191F, G265Y, S94D, E95K, A343K, A350K, F176D, N196D, H147D, H146D, H147E, H146E, T91E, R250E, T91D, R251E, R148D, H254E, A170K, R250K, R251K, H254D, A170R, H146K, H147K, K346E, T90D, R250D, T90E, A138K, R251D, H262K, A169K, H146R, I102E, T90K, A271K, K346R, H306E, D124E, K326E, A169R, D124R.
   64. Vaccine, composition, method or DsA1 and/or DsA2 derivative according to any one of embodiments 1 to 63, further comprising a PITP derivative, wherein the PITP derivative comprises one or more, preferably one, two or three, especially one, of the following amino acid exchanges:
   T392F, F352L, T392I, M303F, A276Y, S365I, T392L, M303W, A276L, T392W, K363T, A361V, F286Y, A361I, K363F, M303Y, T392V, A361L, S365V, A270V, A276M, S365L, K363V, M312L, T252L, M387L, Y297F, A276F, G353T, A276W, K363S, T252R, L314I, T252I, A361F, L295I, L255I, A276I, K363L, S318W, V354L, K363I, A361T, S318I, A361M, G353L, T392Y, S365A, A270I, A361W, T252M, K363A, K363W, L319I, G353D, A276V, G353N, G353F, T252V, G353S, S318V, L255V, G364I, L394V, K363M, S318F, L390I, G353I, S335Y, G364L, S365M, T252Q, S365F, L330A, T252F, S318M, K363Y, G353V, S365G, S365W, K363G, T392M, V354F, A276T, 0364W, K363C, P317I, S335C, P317L, P317F, G269C, S335A, P317V, G268C, S334V, S335V, G269V, S334C, A361C, G268I, G269F, G268L, S334I, G268V, G269L, S335G, S334A, G268F, S335T, P317C, P317M, S335L, A361Y, G269I, S335I, G268Y, K363P, G268W, S335M, K363H, F286I, G268M, G269M, G269W, G268A, S334G, S334L, G268P, G268T, G293F, F286L, T252C, S335N, T392C, K363N, S334F, M303I, K363Q, A376L, G364V, W245I, G269S, Y297C, S335Q, G358V, W245L, A376I, S334M, G358L, T252A, S318C, S335H, Y297L, S365C, M312I, G373L, Y297I, G358I, S335F, G268N, M303L, G269T, Y297V, P317A, G268Q, G268H, G293L, S334T, G305C, S318L, S335D, S335P, G364C, W245V, W245F, P317Y, T392A, F286V, S335W, A376V, G358C, K363D, M312F, G269N, S335R, A378I, A378C, P317W, G268K, G268R, G268D, A376C, G373V, M312V, M303V, G353C, S318A, G364F, G241C, G268E, G379I, G269P, F286C, L330F, G284V, W245C, A378V, L330I, G293I, G373C, G379L, S318Y, G358F, L394I, L330V, G373I, F352I, A361G, S335K, G246C, M387F, A378L, G241I, V354C, M387I, G269A, G308C, F352C, A276C, G293C, G269Q, K363R, G379V, F286W, W245M, G246V, A270C, G246L, M303C, G293V, G305A, Y297A, G345I, G241V; N388D, R248K, S336D, T372E, T249E, E316D, D348E, R382E, R382K, K265E, H327K, D338E, K333R, P386E, S336E, K341D, K341E, K341R, E385D, G268K, G268R, G268D, K340R, G268E, K333D, K368R, G353D; H306I+S335R, H306L+S335R, H306I+S335K, H306C+S335R, H306C+S335R, H306I+S335K, H306L+S335K, H306L+S335K, H306L+S335R, H306V+S335R, H306I+S335R, H306V+S335R, H306C+S335K, H306F+S335K, H306V+S335K, H306V+S335K, H306F+S335R, H306F+S335R, H306W+S335R, H306F+S335K, H306M+S335R, H306W+S335K, H306W+S335R, H306M+S335K, H306A+S335R, H306A+S335R, H306W+S335K, H306A+S335K, H306Y+S335R, H306Y+S335R, H306Y+S335K, H306T+S335R, H306Y+S335K, H306T+S335R, H306T+S335K, H306S+S335R, H306T+S335K, H306S+S335K, S334V+S335R, H306S+S335K, S334V+S335K, S334I+S335R, S334C+S335R, H306P+S335R, M312I+S335R, S334I+S335K, S334V+S335R, S334V+S335K, H306P+S335K, S334C+S335R, S334A+S335R, M312F+S335R, M312L+S335R, S334C+S335K, H306R+Y381L, M312L+S335R, S250L+S335R, H306Q+S335R, H306N+S335K, S334C+S335K, S334A+S335K, H306P+S335K, H306Q+S335R, M312V+S335R, M312I+S335K, S334I+S335K, M312I+S335R, M312L+S335K, H306N+S335R, S334L+S335R, S334A+S335K, M312L+S335K, S250I+S335R, H306N+S335R, S250V+S335R, H306N+S335K, M312F+S335K, S250V+S335R, S250A+S335R, S334M+S335R, M312V+S335R, S250A+S335K, M312F+S335K, S250A+S335K, S250L+S335R, S334G+S335R, S250C+S335R, S250C+S335R, H306Q+S335K, S334G+S335R, Y254F+S335R, S334L+S335R, S2501+S335R, I309C+S335R, S2501+S335K, Y254C+S335R, S250L+S335K, M312V+S335K, S334G+S335K, S250L+H306R, S250V+H306R, H306R+Y381I, M312V+S335K, S2501+H306R, M312C+S335R, Y254C+S335R, S250V+S335K, Y254W+S335R, S334G+S335K, S250L+H306R, S250L+S335K, Y254W+S335R, S250V+S335K, S334L+S335K, I309C+S335R, I309C+S335K, Y254F+S335R, S250A+S335R, S250C+S335K, S250I+H306R, S250A+H306R, S250C+S335K, H306Q+S335K, Y254C+S335K, S334L+S335K, S250A+H306R, I309C+S335K, Y254W+S335K, Y254C+S335K, Y254I+S335R, H306R+Y381C, S250M+S335K, M312C+S335K, M312C+S335K, S250V+H306R, S250M+S335R, Y254V+S335R, Y254I+S335K, S250W+S335R, I259C+S335R, S334M+S335R, Y254A+S335R, Y254V+S335R, S250M+S335K, F251L+S35R, Y2541+H306R, I259C+S335K, Y254I+S335R, I259C+S335K, S250Y+S335K, Y254F+S335K, S250M+S335R, Y254F+H306R, Y254V+S335K, Y254V+H306R, S334T+S335K, S334Y+S335R, Y254A+S335R, S334M+S335K, Y2541+H306R, Y254M+S335K, Y254A+S335K, S334F+S335R, S334M+S335K, F251L+H306R, S334T+S335R, Y254I+S335K, Y254A+S335K, S334T+S335K, S250Y+H306R, Y254A+H306R, Y254M+S335K, F251L+S335K, I259C+S335R, F251L+H306R, Y254M+H306R, S334P+S335R, S334P+S335R, S334P+S335K, S334F+S335R, Y254M+S335R, H306I+S335C, H306L+S335C, H306L+S335C, H306I+S335C, H306V+S335C, H306C+S335C, H306I+S334V, H306V+S335C, H306C+S335C, H306I+S334V, H306L+S335V, H306M+S335C, H306I+S335G, H306I+S335G, H306I+S335A, H306I+S335A, H306F+S335C, H306F+S335C, H306L+S335V, H306I+S335T, H306L+S335A, H306C+S335A, H306I+S335V, H306I+S335V, H306L+S334V, H306L+S335A, H306C+S335V, H306L+S334C, H306I+S335I, H306I+S334A, H306L+S334C, H306L+S334V, H306F+S334V, H306C+S335A, H306F+S335V, H306C+S334V, H306C+S334V, H306I+S335L, H306C+S335V, H306L+S334I, H306M+S335C, H306I+S334C, H306I+S335I, H306W+S335C, H306V+S335A, H306I+S334C, H306I+S334I, H306L+S335I, H306V+S335A, H306C+S334C, H306L+S335G, H306C+S334C, H306L+S335T, H306I+S334I, H306V+S334V, H306I+S335L, H306M+S335V, H306V+S335V, H306V+S335V, H306I+S335M, H306L+S335T, H306V+S335I, H306C+S335G, H306L+S335L, H306C+S334I, H306L+S335Y, H306L+S335I, H306V+S334V, H306C+S335G, H306I+S334A, H306L+S334A, H306C+S335I, H306F+S335V, H306I+S335T, H306L+S335Y, H306C+S334I, H306L+S334I, H306V+S3350, H306V+S335G, H306A+S335C, H306A+S335C, H306M+S335A, H306C+S334A, H306V+S335I, H306L+S335G, H306L+S334A, H306C+S334A, H306I+S335Y, H306I+S335Y, H306W+S335C, H306F+S335A, H306F+S335A, H306F+S335I, H306I+S335N, H306L+S335L, H306I+S335M, H306I+S335N, H306V+S334I, H306V+S334C, H306V+S335L, H306C+S335L, H306C+S335L, H3061+A376L, H306F+S335T, H306L+S335M, H306F+S334V, S334V+S335C, S335C+Y381L, S335C+A376I, S334I+S335C, S334V+S335C, M312I+S335C, M312I+S335C, S335C+A376L, S334I+S335C, S335C+A376L, S335C+A378C, S335C+A376C, S335C+A376V, S334A+S335C, S335C+A378C, S334A+S335C, S335C+A3761, S334V+S335A, S335A+A376L, S335C+A376C, S335C+A376V, S335C+A378L, S334V+S335A, S335A+A376I, S334V+A376L, S334I+S335V, S335C+A378I, M312F+S335C, M312F+S335C, S334I+S335A, S335C+A378V, S335V+A376L, S334V+S335L, S335C+A378I, S334C+S335V, S334V+A376I, S334V+A376I, S335V+A376I, M312I+S334V, S334V+S335I, S335C+Y381I, M312V+S335C, S335C+A378L, S335A+Y381L, S334C+A376C, S334V+S335I, S335C+Y381V, M312L+S335C, S335C+A378V, S335V+A376L, S334I+S335V, S335C+Y381C, S335V+A376I, S334C+A376L, S334C+Y381L, M312V+S335C, S334V+S335L, S334C+A376I, S334C+A376C, S334V+Y381L, M312L+S335C, S335V+A376V, S3341+A376I, S335V+A376V, S335V+Y381L, Y254F+S335C, L310F+S335C, S3351+A376I, S334V+A376V, M312I+S335V, S334C+S335A, S334I+A376L, M312F+S335V, S335C+Y381A, L310F+S335C, S334G+S335C, S334G+S335C, S334V+A376V, S335C+A376M, S335C+A376M, S334A+S335I, S334V+A376L, M312C+S335C, S334C+S335A, S335A+A376V, S334A+S335V, S334A+S335V, Y254F+S335C, S335A+A376L, S335A+Y381I, S335A+A376I, S334L+S335C, S335A+A376V, M312C+S335C, D152F, D152L, D152I, D152V, D152M, D152W, D152Y, D152S, D152A, D152T, D152G, D152N, D152Q, D152P, D152H, D152K, D152R, D152C, D155Q, D155L, D155F, D155I, D155M, D155W, D155V, D155Y, D155A, D155N, D155T, D155S, D155P, D155G, D155H, D155R, D155K, D155C, D156L, D156F, D156I, D156W, D156V, D156A, D156Y, D156M, D156G, D156S, D156T, D156Q, D156N, D156P, D156H, D156K, D156R, D156C, H146R, H146K, H153R, H153K, Y63A, H146A, H153A, F74A, L141A, F81A, P72A, K144A, T143A, Y75A, W98A, D156A, R158A, S250W, S250F, S250D, S250E, S250K, S250R, F251 A, Y254A, I259W, I259F, I259D, I259E, I259R, I259K, H306A, 1309W, I309F, I309E, I309D, I309R, I309K, L310W, L310F, L310D, L310E, L310R, L310K, M312W, M312F, M312D, M312E, M312R, M312K, S334W, S334F, S334D, S334E, S334R, S334K, S335W, S335F, S335D, S335E, S335K, S335R, M337W, M337F, M337D, M337E, M337K, M337R, A376W, A376F, A376E, A376D, A376R, A376K, F377, A378W, A378F, A378D, A378E, A378K, A378R, F380A, Y381A, D348I, D348L, D348M, D348F, D348V, D348Y, D348A, D348W, D348T, D348G, D348P, D348H, D348S, D348R, D348K, D348E, D348C, H306R, H306D, H306E, H306K, H307D, H307R, H307E, H307K, H327R, H327K, H327E, H327D, H302E, H302K, H302R, H302D, H215K, H215R, H215E, H215D, H223K, H223R, H223E, H223D, H403K, H403R, H403E, H403D.
   65. Vaccine, composition, method or DsA1 and/or DsA2 derivative according to any one of embodiments 1 to 64, comprising a polypeptide with at least one P. acnes epitope of the polypeptide according to SEQ ID NO: 49, wherein the polypeptide with at least one P. acnes epitope of the polypeptide according to SEQ ID NO: 49 preferably comprises one or more, preferably one, two or three, especially one, of the following amino acid exchanges compared to SEQ ID NO: 49:
   A274Y, S26C, S26L, A274F, S261, 0119H, A274L, A274W, S26V, S26M, S212L, T108I, A134L, S26F, N130V, A229L, A274I, G194F, S26W, A291F, G181I, A229I, E278I, A175L, G181L, A152L, A274V, N130L, E278V, K46I, E278L, E278A, K46C, K46L, K93L, K93V, E164I, E278C, E164V, E278M, N130I, K46V, R221C, K93C, K46M, S51L, K93I, E278F, K93M, E164A, S5I, R221L, P209L, K46F, S51V, E164C, R221I, E164M, K93F, D137L, P209V, E164L, P59L, R221F, N130C, S51C, R221G, E278T, K46G, P59V, K46A, N130M, E278G, D137V, K46T, P209C, P591, P209I, T144L, N130F, E164F, S51F, R221M, P209F, S51M, R221S, P209A, K93A, P209M, E164Y, D137C, R221A, P115C, P59F, S208A, K46S, P115V, A1341, P115A, S212V, N249L, E278S, D137F, D137M, P115L, S212I, R221V, N249V, D137I, E278Y, G194L, P59M, R221T, A188L, N249I, T144I, E164T, S208C, S208V, D137A, K46Y, K93G, G87I, E164G, K46P, P59C, A97V, S212C, G83L, K93T, N249C, A134V, G83V, T108V, T112L, T144V, A100L, G87V, E164W, E164S, A171L, A97L, A188F, K93W, T901, A174I, K93Y, AlIIL, A971, N130A, 048I, E278W, R221Y, G135L, T112V, F197V, T144F, K93S, G194V, G87A, A188V, T144C, K46N, P115M, A175I, N130T, S51A, G194C, G48L, G104V, A174V, G158V, T90V, R221N, A202L, A39L, A134F, D137Y, G158I, A174L, P155V, A171V, S51T, F197I, T112C, D137W, G135V, D137G, P115G, T250C, S208L, A100I, T250L, T144M, T108C, P115F, G69L, T112F, G181F, A100F, S208I, G223V, T250V, N249M, A111V, N249Y, A111F, A188M, A111I, P209Y, A175V, P1551, N130G, S120V, A111C, A100C, A97F, G194M, 069I, E278Q, S2080, G181C, G87F, G48V, A97C, I205L, G194I, DI37T, S51G, F197C, G223L, N249A, S51Y, P115S, A2021, P155C, S26A, G83M, S120C, D137S, E278N, G83F, G158C, P59Y, G87C, G87M, N130S, P115I, A202F, A100M, G83C, S212M, A39V, A1881, F197L, G181M, A134C, P115T, A174F, K93P, P209W, P209G, P59A, A39C, G104I, T250G, S208F, R221Q, G181A, G104C, T90C, K46Q, P209T, K46W, A171I, G158A, A134M, G48C, G194A, T90A, T90M, N130Y, N249F, A172L, A152V, A1521, P59W, R221P, G158F, T112M, G135M, G181V, G104L, S208T, N249W, A97M, G135F, K93Q, G42L, A111M, G104A, E278P, T90L, R221D, A175C, A188C, A291L, S212A, F123L, T250I, A174C, E164Q, P155L, A174M. G158M, A172V, E164N, A168L, T90F, A39F, G42V, I205F, A39M, N249G, A175F, G135C, A171F, S212F, K46D, A100Y, G421, P59G, G69A, E164P, G158L, G42C, A171C, T250M, T108L, K93N, A171M, G223I, A175M, T108M. A1721, S51P, A291I, A111Y, G20V, S120I, P59T, A58I, G223C, S208M, G48M, K46H, A62L, A43I, T250A, N130W, G194Y, R221H, D137P, N249S, G104F, A100V, F123I, A172C, T112I, G69F, P115Y, P59S, A100W, G104M, G69C, R221W, K93H, A204L, G20L, G223F, A168I, D28K, E275K, K77R, D17R, H76D, H75D, H76E, K77D, H75E, P294D, S293D, S293E, D286E, P294E, S292E, D285E, H76R, K77E, K289R, P295D, D285K, R179E, H75R, P295E, P294R, P294K, D286K, D285R, S293R, S293K, R180D, R180K, H183E, R179K, K289E, R180E, A98K, R179D, A99K, S292D, D286R, K290D, H75K, A99E, N256E, K290E, K290R, R239D, A199K, A200K, K255E, H183D, A99R, H235E, A98R, D102K, and A200R.
   66. Vaccine, composition, method or DsA1 and/or DsA2 derivative according to any one of embodiments 1 to 63 wherein the DsA1 domains are defined as follows: NSR: from S29 to I48, CSD1 from I49 to L130, SR1 from G131 to S147, CSD2 from A148 to L267. SR2 from A268 to T277, CSD3 from A278 to K323, the PT repeat region from P324 to T361, and CTR from S362 to F405 in the sequence of Q6A5X9 or any other functional DsA1 sequence comprising all these domains, especially the DsA1 sequences of FIG. 12 comprising all these domains;
   and/or wherein the DsA2 domains are defined as follows: NSR: from A72 to K92, CSD1 from 193 to L174, SR1 from S175 to S191, CSD2 from A192 to L311, SR2 from A312 to T321, CSD3 from A322 to E366, the PT repeat region from P367 to T420, and CTR from H421 to A463 in the sequence of Q6A5P9 or any other functional DsA2 sequence comprising all these domains, especially the DsA2 sequences of FIG. 12 comprising all these domains;
   and/or wherein the PITP domains are defined as follows: ENFD from A32 to R164; SR1 from E165 to K237. HbD from V238 to L396, SR2 (including C-terminal LPXT(G) motif (i.e. including LPXT, but not the G)) from S397 to T430, and hLAR from G431 to 1467 in the sequence of Q6A9N1 or any other functional PITP sequence comprising all these domains, especially the PITP sequences of FIG. 12 comprising all these domains.
   67. Pharmaceutical composition according to any one of claims 1 to 64 comprising in a liquid formulation or in a dry or frozen formulation which is reconstitutable to a liquid formulation: 0.01 mg/ml to 50 mg/ml, preferably 0.05 to 5 mg/ml, especially 0.1 to 1 mg/ml, polypeptide as defined above comprising at least one P. acnes epitope, 0.1 to 100 mM, preferably 0.5 to 50 mM, especially 0.1 to 10 mM, NaH₂PO₄, 0.1 to 100 mM, preferably 0.5 to 50 mM, especially 0.1 to 10 mM, Na₂HPO₄, 0.1 to 100 mM, preferably 0.5 to 50 mM, especially 0.1 to 10 mM, KH₂PO₄, 1 to 1000 mM, preferably 5 to 500 mM, especially 50 to 300 mM, NaCl, 0.1 to 100 mM, preferably 0.5 to 50 mM, especially 0.1 to 10 mM. KCl, and/or 0.01 to 1%, preferably 0.05 to 0.5%, especially 0.1 to 0.5%, aluminium adjuvant (aluminium hydroxide).
   68. Pharmaceutical composition according to any one of claims 1 to 67 having in a liquid formulation or in a dry or frozen formulation which is reconstitutable to a liquid formulation a pH of 4.5 to 7.4, preferably of 4.7 to 7.3, even more preferred of 5.0 to 7.3, especially of 5.3 to 7.2.
   69. Pharmaceutical composition comprising a P. acnes polypeptide or vaccine according to any one of claims 1 to 68, eliciting a cross-type reactivity at least against Types IA1, IA2, and II of P. acnes and/or eliciting a cross-type reactivity at least against Types IA1, IA2, IB, II and III of P. acnes.
   70. Pharmaceutical composition comprising a polypeptide as defined in any one of claims 1 to 69, wherein the polypeptide is present in the composition at least partially in a form wherein it lacks an N-terminal methionine residue.
   71. A vaccine comprising Dermatan sulfate-binding adhesin 1 of P. acnes (DsA1 polypeptide) and/or Dermatan sulfate-binding adhesin 2 of P. acnes (DsA2 polypeptide), and/or a fragment and/or a derivative of DsA1 or DsA2, wherein DsA1 and DsA2 comprise from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Thr repeat containing region (“PT repeat region”), and a C-terminal region (“CTR”), wherein the fragment and/or the derivative comprises or consists at least of a CSD2 fragment, wherein the CSD2 fragment is preferably
a contiguous polypeptide sequence of phenylalanine 150 (F150) to leucine 184 (L184),
a contiguous polypeptide sequence of phenylalanine 150 (F150) to leucine 267 (L267), or
a contiguous polypeptide sequence of histidine 218 (H218) to leucine 267 (L267), with the proviso that the fragment or derivative does not comprise an NSR and a CTR, and wherein the amino acid numbering corresponds to the amino acid sequence Q6A5X9 in the UniProt database.
   72. Vaccine according to embodiment 71, wherein the fragment or the derivative comprises or consists at least of
a contiguous polypeptide sequence of phenylalanine 150 (F150) to isoleucine 193 (1193),
a contiguous polypeptide sequence of phenylalanine 150 (F150) to leucine 267 (L267), or
a contiguous polypeptide sequence of histidine 218 (H218) to leucine 267 (L267).
   73. Vaccine according to embodiment 71 or 72, with the proviso that the fragment or the derivative does further not comprise a CSD1 of DsA1 or DsA2, an SR1 of DsA1 or DsA2, an SR2 of DsA1 or DsA2, a CSD3 of DsA1 or DsA2, or a PT repeat region of DsA1 or DsA2, preferably with the proviso that the fragment or derivative does further not comprise a CSD1 of DsA1 or DsA2, an SR1 of DsA1 or DsA2, an SR2 of DsA1 or DsA2, a CSD3 of DsA1 or DsA2, and a PT repeat region of DsA1 or DsA2.
   74. Vaccine according to any one of embodiments 71 to 73, wherein the fragment is
   (1) a contiguous polypeptide sequence of phenylalanine 150 (F150) to leucine 184 (L184), further extending one, two, three, or four amino acids at the N-terminus and/or one, two, three, four, five, six, seven, eight, nine, or ten amino acids at the C-terminus;
   (2) a contiguous polypeptide sequence of phenylalanine 150 (F150) to leucine 267 (L267), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus; and/or
   (3) a contiguous polypeptide sequence of histidine 218 (H218) to leucine 267 (L267) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus.
   75. Vaccine according to any one of embodiments 71 to 74, wherein the fragment has a length of at least 35 amino acids, preferably a length of at least 40 amino acids, especially a length of at least 50 amino acids.
   76. Vaccine according to any one of embodiments 71 to 75, which comprises at least one additional polypeptide with an amino acid sequence containing or consisting of at least one additional P. acnes antigen or P. acnes epitope which is not a DsA1 or DsA2 antigen or epitope, preferably at least one additional amino acid sequence containing at least the putative iron-transport protein (PITP) polypeptide of P. acnes or at least one epitope of PITP polypeptide of P. acnes, wherein the PITP polypeptide comprises from N- to C-terminus an extended neocarzinostatin family domain (“ENFD”), a first swapping region (“SR1”), a heme-binding domain (“HbD”), a second swapping region (“SR2”) including the C-terminal LPXTG motif, and a hydrophobic C-terminal region (“hLAR”).
   77. Vaccine according to embodiment 76 wherein the derivative comprises at least one additional sequence from the PITP polypeptide, preferably a sequence comprising at least one of ENFD, SR1, HbD, and SR2, preferably selected from ENFD and HbD.
   78. Vaccine according to any one of embodiments 71 to 77 comprising a derivative wherein the derivative comprises at least one fragment of DsA1 and at least one fragment of DsA2.
   79. Vaccine according to any one of embodiments 71 to 78, wherein in the DsA1/DsA2 fragment or derivative at least 5 PT repeats, preferably at least 10 PT repeats, especially at least 15 PT repeats, are deleted compared to a naturally occurring wild type DsA1/DsA2 polypeptide (“native DsA1/DsA2”), and wherein preferably at least one, more preferred at least two, more preferred at least three, even more preferred at least four, especially five, PT repeat(s) is/are present.
   80. Vaccine according to any one of embodiments 71 to 79, wherein the derivative further comprises at least an NSR, CSD1, SR1, CSD2, SR2, CSD3 and/or CTR from at least one other DsA1 and/or DsA2 from at least one other P. acnes strain than the strain from which the CSD2 fragment has been derived.
   81. Vaccine according to any one of embodiments 71 to 80, wherein the fragment or derivative lacks the NSR and/or the CTR.
   82. Vaccine according to any one of embodiments 71 to 81 further comprising another P. acnes antigen or epitope, preferably an antigen selected from DsA2 and/or PITP and/or a DsA2 epitope and/or a PITP epitope.
   83. Vaccine according to any one of embodiments 71 to 82 further comprising an epitope containing fragment of another P. acnes polypeptide, preferably an epitope containing fragment of DsA2 and/or PITP.
   84. DsA1 and/or DsA2, and/or a fragment and/or a derivative of DsA1 or DsA2 according to any one of embodiments 71 to 83 for use in a therapeutic treatment, preferably for use in the treatment or prevention of P. acnes-associated infections, especially selected from the group consisting of acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes.
   85. DsA1 and/or DsA2, and/or a fragment and/or a derivative of DsA1 or DsA2 according to any one of embodiments 71 to 83 for use in the treatment or prevention of P. acnes-associated infections in a human patient suffering from P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes.
   86. Pharmaceutical formulation, especially vaccine, according to any one of embodiments 1 to 85, comprising a PITP polypeptide and/or a porphyrin-binding PITP fragment and/or a porphyrin-binding PITP derivative, further comprising a porphyin molecule and/or a porphyrinogen molecule and/or a porphyrinogen or porphyrin degradation product bound to the PITP and/or a porphyrin-binding PITP fragment and/or a porphyrin-binding PITP derivative, wherein the porphyin molecule and/or porphyrinogen molecule and/or porphyrinogen or porphyrin degradation product preferably contains a metal ion in complex form, wherein the metal ion is preferably a Fe, Co, Zn, Mg, Se, or Cu ion, especially a Fe²⁺ or a Fe³⁺ ion.
   87. Pharmaceutical formulation, especially vaccine, according to any one of embodiments 1 to 86, comprising a PITP polypeptide and/or a porphyrin-binding PITP fragment and/or a porphyrin-binding PITP derivative, further comprising a porphyin molecule and/or a porphyrinogen molecule and/or a porphyrinogen or porphyrin degradation product bound to the PITP and/or a porphyrin-binding PITP fragment and/or a porphyrin-binding PITP derivative, wherein the porphyin molecule and/or porphyrinogen molecule and/or porphyrinogen or porphyrin degradation product is selected from the group hemin, protoprophyrin IX, coproporphyrin I, II and/or III, especially coproporphyrin III; pentacarboxyporphyrin, uroporphyrin, heptacarboxyporphyrin, hexacarboxyporphyrin, especially as form I or III; nitro-porphyrins, especially 5,10,15,20-tetraphenylporphyrin (TPP), 5,10,15,20-tetra(4′-fluorophenyl)porphyrin (TpFPP), 5,10,15,20-tetra(4′-chlorophenyl)porphyrin (TpCIPP), 5,10,15,20-tetra(4′-bromophenyl)porphyrin (TpBrPP), biliverdin, 5,10-diphenyl-15,20-di(N-methylpyridinium-4-yl)porphyrin (Di-cis-Py+), PORF-1 to PORF-34, porphine, octaethylporphyrin, tetraphenylporphyrin, verteporfin, ascoproporphyrin, hydroxymethylbilane (HMB), mesoporphyrin IX, 7-carboxyporphyrin (7P), 6-carboxylporphyrin (6P), heme A, heme B, heme C, heme O, heme I, heme m, heme D, S heme; uroporphyrinogen III, chlorophylls, especially chlorophyll a, chlorophyll b, and bacteriochlorophylls.
   88. Vaccine or pharmaceutical formulation as defined in any one of embodiments 1 to 87 for use in the treatment or prevention of P. acnes-associated infections, comprising a polypeptide comprising an epitope of DsA1 and/or DsA2 and/or PITP, wherein the epitope is selected from the group consisting of R32-I141, Q38-K51, R32-K51, T43-K51, Q38-K51, R87-K90+T43-K51, R87-K90+T117-I132 and R87-K90+S234-G250, R87-K90+L246-A260, R87-K90+A256-E270, R87-K90+R266-T277, T117-I132, T117-A127, V128-I132. A144-N157. H146-A160, A148-N157. A156-A170, K166-L180, A176-T190, P186-A198, N181-E191, I216-F224. I216-D225, A226-A24G, S234-G250, I251-I263, I264-P271, P236-G250, L246-A260, A256-E27G, S234-G250, I251-I263, I251-L267, A268-L280, R266-T277, T285-R286+I216-F224, T285-R286+I264-P271, T285-R286+V289-K296, T285-R286+V289-K296, T285-R286+A144-N157, A310-D313+T285-R286, T285-D290, T285-D290+V291-T300, T285-D290+A301-E307, V291-T300, A301-E307, T285-T300, A301-E307, R286-D290+V291-T300, R286-D290+A301-E307, R286-T300, V289-K296, A310-D313+I216-F224, A310-D313+I264-P271, A310-D313+T285-R286, A310-D313+R286-D290, A310-D313+V289-K296. A310-D313+V289-K296, A310-D313+T285-T300, A310-D313+A144-N157, A310-D313+T285-R286, A310-D313+T285-D290, A310-D313+T293-E307, T285-D290, V291-T300, T293-E307, A301-E307 of DsA1; L152-Q166, G190-P230, I199-D208, A218-I237, P230-Q244, I231-A270, H254-A270, A271-S279, A271-R310, L311-T321, L311-K323 V333-Q347, A218-P230, I231-I237, H254-H262, Q256-H262, E261-D269, D269-S279, K313-K323, of DsA2; D79-T90, E73-D85, R43-150, P68-Y75, P86-E92, 139-G45, Y84-D89, F81-D89, D79-T90, T37-E44, E73-W98, E73-F8I, D89-T90, P72-F8I, A129-F138, D120-Q134, F111-D120, F132-G147, D152-E165, R115-F123, D120-K128, P131-F138, N181-E191, T143-T159, P116-T124, P131-D137, P131-D137, T175-C231, Q198-K203, P179-K185, G200-Q210, K174-A188, K174-K185, P201-Q209, P183-P201, P183-K191, K185-P195, R164-S180, E165-S180, K185-S190, V193-N202, V193-G200, K203-P208, R216-T225. R216-R224, P173-K191, K197-K203, P168-T175, K185-K203. R164-K174. T175-V193, S250-N261, D287-S300, K340-V347, D338-F352, D338-D348, S285-P288+G305-L314, S285-P288+H306-L314, S285-P288+T342-T351, S285-P288+D338-D348, D287-S300, T342-T351, D338-D348, H306-L314, 0305-L314, G364-K375, R382-E399, V367-G373, A383-L390, T342-T351, M387-T395, E385-T392, V401-V410, N404-A409, G416-L427. L396-V410, T406-I415, D417-G424, V407-D418, V407-V414, K421-V429, S419-T430, D408-1415, T406-V414, of PITP, preferably wherein the epitope comprises at least one additional amino acid residue at the N- or C-terminus of the DsA1, DsA2, or PITP sequence, especially wherein the epitope comprises at least two additional amino acid residue at the N- or C-terminus of the DsA1, DsA2, or PITP sequence; wherein the polypeptide is preferably covalently linked to a carrier molecule molecule or embedded in a scaffold, especially a carrier polypeptide.
   89. Vaccine or pharmaceutical formulation as defined in any one of embodiments 1 to 87 for use in the treatment or prevention of P. acnes-associated infections, comprising a polypeptide comprising an epitope of DsA1 and/or DsA2 and/or PITP, wherein the polypeptide is a DsA1/DsA2/PITP derivative with the motif K[NYI] starting at position 5 or 6 of the polypeptide and/or having on DNA level the sequence AADUAU or AAVAUU at this coding position.
   90. Vaccine or polypeptide according to any one of embodiments 1 to 89, wherein the DsA1 and/or DsA2 and/or PITP, and/or a fragment or derivative of DsA1 or DsA2 or PITP is provided as DNA or RNA encoding an antigen or an epitope containing fragment or derivative thereof, preferably as mRNA vaccine, especially an mRNA vaccine formulated with cationic polymers, with an mRNA molecule with the following structure: 5′UTR-signal peptide-encoded antigen or epitope-3′UTR; or as a vector-based vaccine, especially with an adenovirus vector, an adeno-associated virus vector, a vesicular stomatitis virus vector, a Newcastle Disease virus vector, an alphavirus vector, a baculovirus vector, or a retrovirus vector; and/or wherein the coding sequence on DNA level has the initial coding DNA sequence ATGGTG; especially wherein the mRNA comprises a sequence as transcribed from SEQ ID NOs: 67 to 76.
   91. Pharmaceutical formulation comprising a vaccine or polypeptide according to any one of embodiments 1 to 90 comprising the polypeptide as defined in any one of these claims, i.e. a DsA1 and/or DsA2 and/or PITP, and/or a fragment and/or a derivative of DsA1 and/or DsA2 and/or PITP according to any one of embodiments 1 to 90 or mixtures thereof; and a pharmaceutically acceptable excipient or carrier.
   92. Method of treatment or prevention of P. acnes-associated infections in a human patient comprising administration of an effective amount of a DsA1 and/or DsA2 and/or PITP, and/or a fragment and/or a derivative of DsA1 and/or DsA2 and/or PITP according to any one of embodiments 1 to 91 or mixtures thereof in an effective amount to a patient in need thereof.
   93. Method of treatment or prevention of P. acnes-associated infections in a human patient suffering from P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes comprising administration of an effective amount of a DsA1 and/or DsA2 and/or PITP, and/or a fragment and/or a derivative of DsA1 and/or DsA2 and/or PITP according to any one of embodiments 1 to 91 or mixtures thereof in an effective amount to a patient in need thereof.
   94. Method according to claim 93, wherein administration is performed by intradermal, subcutaneous (s.c.), parenteral, intramuscular (i.m.), mucosal, transcutaneous or topical administration, preferably by intradermal, or intramuscular administration, especially by a syringe or by microneedeling devices.
   95. Method of production of the polypeptides, vaccines and formulations according to any one of embodiments 1 to 91, wherein the polypeptides comprising at least one P. acnes epitope as defined in these claims are expressed in a host cell, extracted and puriied from these host cells, and, optionally, formulated and finished to a pharmaceutical formulation, especially a vaccine for use in the treatment or prevention of P. acnes-associated infections in a human patient.
   96. Vaccine according to any one of embodiments 1 to 91 comprising a polypeptide
(a) comprising at least an epitope-containing fragment or derivative of CSD1 of DsA1 and at least an epitope-containing fragment or derivative of CSD2 of DsA2; and/or
(b) comprising at least a polypeptide stretch comprising a CSD1 fragment or derivative of CSD1 of DsA1 of at least 30 amino acid residues, preferably at least 40 amino acid residues, and at least a polypeptide stretch comprising a CSD2 fragment or derivative of CSD2 of DsA1 of at least 30 amino acid residues, preferably at least 40 amino acid residues;
(c) comprising at least
   a contiguous polypeptide sequence of phenylalanine 150 (F150) to leucine 184 (L184),
   a contiguous polypeptide sequence of phenylalanine 150 (F150) to leucine 267 (L267), or
   a contiguous polypeptide sequence of histidine 218 (H218) to leucine 267 (L267) of DsA1, and
   a contiguous polypeptide sequence of phenylalanine 194 (F194) to leucine 228 (L228),
   a contiguous polypeptide sequence of phenylalanine 194 (F194) to leucine 311 (L311), or
   a contiguous polypeptide sequence of histidine 262 (H262) to leucine 311 (L311) of DsA2,
especially wherein the polypeptide comprises an amino acid exchange at one or more of C53, C319 and C321 of DsA1, and C97 and C363 of DsA2, if present in the polypeptide, preferably one or more of amino acid exchanges C53S, C319S and C321P of DsA1 and C97S and C363S of DsA2 and/or wherein in the fragment or derivative of DsA1 or DsA2 at least 5 PT repeats, preferably at least 10 PT repeats, especially at least 15 PT repeats, are deleted compared to a naturally occurring wild type DsA1/DsA2 polypeptide (“native DsA1/DsA2”), and wherein preferably at least one, more preferred at least two, more preferred at least three, even more preferred at least four, especially five, PT repeat(s) is/are present.
   97. Vaccine according to any one of claims 1 to 96, wherein the polypeptide further comprises a PITP polypeptide or a fragment or derivative of PITP comprising a polypeptide stretch of at least 30 amino acid residues comprising at least an epitope of PITP, preferably wherein the PITP polypeptide or a fragment or derivative of PITP comprises at least
a contiguous polypeptide sequence of proline 34 to glutamic acid 73 of ENFD or a PITP derivative thereof.
a contiguous polypeptide sequence of proline 94 to threonine 143 of ENFD or a PITP derivative thereof, or
a contiguous polypeptide sequence of valine 238 to asparagine 393 of HbD or a PITP derivative thereof;
especially wherein the PITP derivative comprises an amino acid exchange exchange at positions C231 and C402.
   98. Method of production of the polypeptides, vaccines and formulations according to any one of embodiments 1 to 91, wherein the polypeptides comprising at least one P. acnes epitope as defined in these claims are expressed or produced as an mRNA in a host cell, extracted and purified from these host cells, and, optionally, formulated and finished to a pharmaceutical formulation, especially a vaccine for use in the treatment or prevention of P. acnes-associated infections in a human patient.
   99. Method according to embodiment 98, wherein the host cell is an eukaryotic or prokaryotic host cell, preferably a prokaryotic host cell, especially E. coli or L. lactis: or a host cell which is able to glycosylate the expressed polypeptides as defined in any one of embodiments 1 to 96.
   100. Method according to embodiment 98 or 99, wherein the host cell is naturally unable to synthesize porphyrins and wherein preferably porphyrins are added to the culture, to the lysis buffer, and/or to the purified protein.
   101. Method according to any one of embodiments 98 to 100, wherein the polypeptides of SEQ ID NOs:1 to 66 or encoded by SEQ ID NOs:67 to 76 are expressed in the host cells.

Therefore, the present invention also discloses the following embodiments, specifically drawn i.a to the aspect of the present invention wherein PITP is used in a product, preferably in a pharmaceutical product, especially in a vaccine comprising PITP, at least one PITP fragment or fusion polypeptides with at least one immunogenic PITP epitope:

1. A vaccine comprising the putative iron-transport protein of P. acnes (PITP polypeptide) and/or a fragment and/or or a derivative of PITP, wherein PITP comprises from N- to C-terminus an extended neocarzinostatin family domain (“ENFD”), a first swapping region (“SR1”), a heme-binding domain (“HbD”), a second swapping region (“SR2”) including the C-terminal LPXT(G) motif, and a hydrophobic C-terminal region (“hLAR”), wherein the fragment and/or the derivative comprises or consists at least of a PITP epitope.
2. Vaccine according to embodiment 1, wherein the fragment and/or derivative is a PITP polypeptide wherein the hLAR is deleted, replaced by a hydrophilic C-terminal region, or partially deleted, wherein the partial deletion results in a loss of hLAR except the N-terminal 12 amino acids of hLAR, preferably except the N-terminal 11 amino acids of hLAR, especially except the N-terminal 10 amino acids of hLAR; or a fragment thereof or derivative thereof comprising at least amino acids corresponding to proline 34 to glutamic acid 73 or proline 94 to threonine 143 of ENFD or valine 238 to asparagine 393 of HbD in the amino acid sequence Q6A9N1 in the UniProt database.
3. Vaccine according to embodiment 1 or 2, wherein the fragment or the derivative comprises or consists at least of
a contiguous polypeptide sequence of proline 34 to glutamic acid 73 of ENFD,
a contiguous polypeptide sequence of proline 94 to threonine 143 of ENFD, or
a contiguous polypeptide sequence of valine 238 to asparagine 393 of HbD.
   4. Vaccine according to any one of embodiments 1 to 3, wherein the fragment or derivative contains a fragment of ENFD and/or of HbD of the PITP polypeptide with a length of at least 8 amino acid residues, preferably at least 9 amino acids, more preferred at least 10 amino acids, more preferred at least 15 amino acids, even more preferred at least 35 amino acids, even more preferred a length of at least 40 amino acids, especially a length of at least 50 amino acids.
   5. Vaccine according to any one of embodiments 1 to 4, which comprises at least one additional polypeptide with an amino acid sequence containing or consisting of at least one additional P. acnes antigen or P. acnes epitope which is not a PITP antigen or epitope, preferably at least one additional amino acid sequence containing at least the Dermatan sulfate-binding adhesin 1 of P. acnes (DsA1 polypeptide) and/or Dermatan sulfate-binding adhesin 2 of P. acnes (DsA2 polypeptide), and/or a fragment and/or a derivative of DsA1 or DsA2, wherein DsA1 and DsA2 comprise from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Thr repeat containing region (“PT repeat region”), and a C-terminal region (“CTR”), wherein the fragment and/or the derivative comprises or consists at least of a CSD2 fragment, wherein the CSD2 fragment is preferably
a contiguous polypeptide sequence of phenylalanine 150 (F150) to leucine 184 (L184),
a contiguous polypeptide sequence of phenylalanine 150 (F150) to leucine 267 (L267), or
a contiguous polypeptide sequence of histidine 218 (H218) to leucine 267 (L267),
   with the proviso that the fragment or derivative does not comprise an NSR and a CTR, and wherein the amino acid numbering corresponds to the amino acid sequence Q6A5X9 in the UniProt database.
   6. Vaccine according to embodiment 5 wherein the derivative comprises at least one additional sequence from the DsA1 or DsA2 polypeptide, preferably a sequence comprising at least one of CSD1, CSD2, CSD3 of DsA1 or DsA2 or an epitope-containing fragment thereof.
   7. Vaccine according to any one of embodiments 1 to 6 or an isolated fragment or derivative of the PITP polypeptide as defined in any one of embodiments 1 to 6, wherein the fragment and/or derivative is a PITP polypeptide wherein the hLAR is deleted, replaced by a hydrophilic C-terminal region, or partially deleted, wherein the partial deletion results in a loss of hLAR except the N-terminal 12 amino acids of hLAR, preferably except the N-terminal 11 amino acids of hLAR, especially except the N-terminal 10 amino acids of hLAR; or a fragment thereof or derivative thereof comprising at least amino acids corresponding to proline 34 to glutamic acid 73 or proline 94 to threonine 143 of ENFD or valine 238 to asparagine 393 of HbD in the amino acid sequence Q6A9N1 in the UniProt database.
   8. Vaccine according to any one of embodiments 1 to 6 or a fragment or derivative of the PITP polypeptide according to embodiment 7, wherein the fragment or the derivative comprises or consists at least of
a porphyrin-binding domain of ENFD or HbD or a prophyin-binding derivative thereof,
a contiguous polypeptide sequence of proline 34 to glutamic acid 73 of ENFD,
a contiguous polypeptide sequence of proline 94 to threonine 143 of ENFD, or
a contiguous polypeptide sequence of valine 238 to asparagine 393 of HbD.
   9. Vaccine according to any one of embodiments 1 to 6 or a fragment or derivative of the PITP polypeptide according to embodiment 7 or 8, wherein the fragment or the derivative comprises or consists at least of an epitope of ENFD and/or an epitope of HbD, preferably wherein the fragment or the derivative comprises or consists at least of an epitope of ENFD and an epitope of HbD.
   10. Vaccine according to any one of embodiments 1 to 6 or a fragment or derivative of the PITP polypeptide according to any one of embodiments 7 to 9, wherein the derivative further comprises at least a ENFD and/or HbD from at least one other PITP from at least one other P. acnes strain than the strain from which the PITP fragment or derivative has been derived.
   11. Vaccine according to any one of embodiments 1 to 6 or a fragment or derivative of the PITP polypeptide according to any one of embodiments 7 to 10, wherein the fragment or derivative lacks the SR, corresponding to serine 180 (S180) to glutamine 198 (Q198), preferably at least the amino acid sequence corresponding to proline 179 (P179) to threonine 207 (T207), especially the amino acid sequence corresponding to glutamic acid 165 (E165) to lysine 237 (K237), or valine 401 (V401) to threonine 430 (T430), preferably serine 397 (S397) to T430, especially leucine 394 to T430; in the amino acid sequence Q6A9N1 in the UniProt database.
   12. Vaccine according to any one of embodiments 1 to 6 or a fragment or derivative of the PITP polypeptide according to any one of embodiments 7 to 11, wherein at least the amino acid sequence corresponding to leucine 427 (L427) to glycine 431 (G431) is deleted, preferably wherein at least the amino acid sequence corresponding to proline 179 (P179) to glycine 431 (G431) is deleted, especially wherein at least the amino acid sequence corresponding to threonine 392 (T392) to glycine 431 (G431), is deleted; according to the numbering in the amino acid sequence Q6A9N1 in the UniProt database.
   13. Vaccine according to any one of embodiments 1 to 6 or a fragment or derivative of the PITP polypeptide according to any one of embodiments 7 to 12, wherein at least the amino acid sequence corresponding to serine 180 (S180) to glutamine 198 (Q198) and/or phenylalanine 74 (F74) to serine 93 (S93), preferably at least the amino acid sequence corresponding to proline 179 (P179) to threonine 207 (T207), especially the amino acid sequence corresponding to threonine 159 (T159) to threonine 219 (T219), in the amino acid sequence Q6A9N1 in the UniProt database is deleted.
   14. Vaccine according to any one of embodiments 1 to 6 or a fragment or derivative of the PITP polypeptide according to any one of embodiments 7 to 12, wherein the fragment or derivative consists of one or more the following amino acids: A32 to T430, A32 to G426, A32 to Q198, A32 to T143, A32 to K400. A32 to T159, A32 to I177, A32 to Q204, A32 to G234, A32 to R164, A32 to S391, A32 to P179. A32 to R158. A32 to G147, A32 to E73 and P94 to G147; P34 to T430, P34 to G426, P34 to Q198, P34 to T143, P34 to K400, P34 to T159, P34 to I177, P34 to Q204, P34 to 0234, P34 to R164, P34 to S391, P34 to P179, P34 to R158, P34 to G147, P34 to E73 and P94 to G147: S240 to S391. A32 to D44I, A32 to I440, A32 to E439, A32 to D438, A32 to S437, A32 to S436, A32 to P435, A32 to G434, A32 to E433, A32 to A432, A32 to G431; P34 to D441, P34 to I440, P34 to E439, P34 to D438, P34 to S437, P34 to S436, P34 to P435, P34 to G434, P34 to E433, P34 to A432, P34 to G431; S240 to D441, S240 to I440, S240 to E439, S240 to D438, S240 to S437, S240 to S436, S240 to P435. S240 to G434, S240 to E433, S240 to A432, S240 to G431; A32 to T430, A32 to V429, A32 to P428, A32 to L427, A32 to G426, A32 to R425, A32 to G424, A32 to A423, A32 to G422. A32 to K421, A32 to G420, A32 to S419, A32 to D418. A32 to D417. A32 to G416, A32 to 1415, A32 to V414, A32 to K413. A32 to G412. A32 to T411, A32 to V410, A32 to A409, A32 to D408. A32 to V407, A32 to T406, A32 to V405, A32 to N404, A32 to H403, A32 to C402, A32 to V401, A32 to K400, A32 to E399. A32 to A398. A32 to S397, A32 to L396, A32 to T395, A32 to L394. A32 to N393, A32 to T392, P34 to T430, P34 to V429, P34 to P428, P34 to L427, P34 to 0426, P34 to R425, P34 to 0424, P34 to A423, P34 to G422, P34 to K421, P34 to G420, P34 to S419, P34 to D418, P34 to D417, P34 to G416, P34 to 1415, P34 to V414, P34 to K413, P34 to G412, P34 to T411, P34 to V410, P34 to A409, P34 to D408, P34 to V407, P34 to T406, P34 to V405, P34 to N404, P34 to H403, P34 to C402, P34 to V401, P34 to K400, P34 to E399, P34 to A398, P34 to S397, P34 to L396, P34 to T395, P34 to L394, P34 to N393, P34 to T392; G172 to T430, G172 to V401. G172 to K400, G172 to L396, G172 to N393, A199 to T430, A199 to V401, A199 to K400, A199 to L396, A199 to N393, H223 to T430, H223 to V401, H223 to K400, H223 to L396, H223 to N393, T232 to T430, T232 to V401, T232 to K400, T232 to L396, T232 to N393. G234 to T430, G234 to V401, G234 to K400, G234 to L396, G234 to N393, V238 to T430, V238 to V401, V238 to K400, V238 to L396, V238 to N393, S240 to T430, S240 to V429, S240 to P428. S240 to L427, S240 to G426, S240 to R425. S240 to G424, S240 to A423, S240 to G422. S240 to K421. S240 to G420, S240 to S419, S240 to D418, S240 to D417, S240 to G416, S240 to I415, S240 to V414, S240 to K413, S240 to G412, S240 to T411, S240 to V410, S240 to A409, S240 to D408, S240 to V407, S240 to T406, S240 to V405, S240 to N404, S240 to H403, S240 to C402, S240 to V401, S240 to K40G, S240 to E399, S240 to A398, S240 to S397, S240 to L396, S240 to T395, S240 to L394, S240 to N393, S240 to T392; 391-481, 40P-49T, 41V-50I, 42G-51S, 43R-52G, 44E-53K, 68P-77N, 69A-78S, 70S-79D, 71V-80K, 72P-81F, 73E-82Y, 74F-83G, 75Y-84Y, 76G-85D, 77N-86P, 78S-87S, 79D-88K, 80K-89D, 81F-90T, 82Y-91T, 83G-92E, 84Y-93S, 85D-94P, 86P-95S, 87S-96T, 88K-97I, 89D-98W, 90T-99V, 91T-100Y, 92E-101T, 93S-102P, 94P-103S, 95S-104Q, 96T-105K, 971-106A, 98W-1071, 99V-108G, 100Y-109S, 101T-110R, 102P-111F, 103S-112A, 104Q-113Q, 105K-114G, 106A-115R, 107I-116P, 108G-117M, 109S-118N, 110R-119N, 111F-120D, I12A-I210, 1251-134Q, I26T-135G, 127M-136K, I28K-137D, 144K-153H, 145A-154S, 146H-155D, I470-156D, 148V-157T, 149G-158R, 150K-159T, 151T-160P, 152D-161V, 153H-162T, 154S-163Y, 155D-164R, 156D-165E, 157T-166A, 158R-167T, 159T-168P, 160P-169A, 161V-170P, 162T-171T, 163Y-172G, 164R-173P, 165E-174K, 166A-175T, 167T-176P, 168P-177I, 169A-178A, 170P-179P, 171T-180S, 172G-181K, 173P-182Q, 174K-183P, 175T-184S, 176P-185K, 177I-186Q, 178A-187A, 179P-188A, 180S-189P, 181K-190S, 182Q-191K, 183P-192Q, 184S-193V, 185K-194K, 186Q-195P, 187A-196S, 188A-197K, 189P-198Q, 190S-199A, 191K-200G, 192Q-201P, 193V-202N, 194K-203K, 195P-204Q, 196S-205S, 197K-206T, 198Q-207T, 199A-208P, 200G-209Q, 201P-210Q, 202N-211K, 203K-212T, 204Q-213A, 205S-214E, 206T-215H, 207T-216R, 208P-217S, 209Q-218Q, 210Q-219T, 211K-220P, 212T-221A, 213A-222A, 214E-223H, 215H-224R, 216R-225T, 217S-226M, 218Q-227T, 219T-228K, 220P-229Q, 221A-230V, 222A-231C, 223H-232T, 224R-233I, 225T-234G, 226M-235A, 227T-236S, 228K-237K, 229Q-238V, 230V-239T, 231C-240S, 232T-241G, 233I-242S, 266L-275S, 267S-276A, 268G-277F, 282T-291K, 334S-343N, 353G-362I, 354V-363K, 355S-364G, 356V-365S, 357S-366P, 358G-367V, 359N-368K, 377F-386P, 378A-387M, 379G-388N, 380F-389P, 396L-405V, 397S-406T, 398A-407V, 399E-408D, 400K-409A, 401V-410V, 402C-411T, 403H-412G, 404N-413K, 405V-414V, 406T-415I, 407V-416G, 408D-417D, 409A-418D, 410V-419S, 411T-420G, 412G-421K, 413K-422G, 414V-423A, 415I-424G, 416G-425R, 417D-426G, 418D-427L, 419S-428P, 420G-429V, 421K-430T, 422G-431G, 423A-432A, 424G-433E, 425R-434G, 426G-435P, 427L-436S, 428P-437S, 429V-438D, 430T-439E, 431G-440I, 432A-441D, 433E-442L, 434G-443G, 435P-444I, 436S-445V, and 437S-446G; 139-K53, P68-G121, I125-D137, K144-S242, L266-F277, T282-K29I, S334-N343, G353-K368, F377-P389, and L396-G446, especially a fragment or derivative comprising or consisting of the fragments selected from the group consisting of the PITP fragments A32 to R164, A32 to Q198, A32 to T143, A32 to V148, A32 to T171, P34 to R164, A32 to T159, A32 to I177, A32 to Q204, A32 to G234, A32 to K400, A32 to S391, V238 to K400, A199 to T430, V238 to T395. G234 to K400, H223 to K400, T232 to V401. V238 to T392, V238 to N393. V238 to L394, V238 to T395, V238 to L396, T232 to T430, G172 to K400, and G172 to G234, and fragments thereof comprising at least of a PITP epitope.
   15. Vaccine according to any one of embodiments 1 to 14, wherein the vaccine is protective against development of a pathological condition induced by P. acnes invasion, preferably wherein the vaccine is formulated for administration to a human subject, e.g. a child, an adolescent or adult subject.
   16. Vaccine according to any one of embodiments 1 to 15, wherein the vaccine is formulated for use in a treatment regimen involving both, a prime and boost immunization, preferably wherein the same formulation is used for the prime and boost administration.
   17. Vaccine according to any one of embodiments 1 to 16, wherein the vaccine is formulated with an adjuvant, preferably with an adjuvant selected from the group consisting of mineral salts, preferably alum (potassium aluminum sulfate), aluminum hydroxide, aluminum phosphate, aluminum oxohydroxide, aluminum hydroxyphosphate, calcium phosphate, cerium nitrate, zinc sulfate, colloidal iron hydroxide, and/or calcium chloride: oil-in-water emulsions, liposomes. TLR agonists, Monophosphoryl Lipid A, saponins, phospholipids, emulsion-based adjuvants, aluminium hydroxide gel, solid phase adsorbents, nanospheres and encapsulating materials such as liposomes; high molecular weight adjuvants, preferably toxins, toxoids or any mutant cross-reactive material of the toxin from tetanus, diphtheria, pertussis, Pseudomonas species. E. coli, Staphylococcus species, and Streptococcus species. Such toxins or toxoids may be tetanus toxoid, pertussis toxoid, cholera toxoid, E. coli LT, E. coli ST, and exotoxin A from Pseudomonas aeruginosa; bacterial outer membrane proteins such as outer membrane protein complex c (OMPC), porins, transferrin binding proteins, pneumolysin, pneumococcal surface protein A (PspA), pneumococcal adhesin protein (PsaA), C. difficile enterotoxin (toxin A) and cytotoxin (toxin B) or Haemophilus influenzae protein D, other pharmaceutically acceptable polypeptide carriers, such as ovalbumin, keyhole limpet hemocyanin (KLH), bovine serum albumin (BSA) or purified protein derivative of tuberculin (PPD); cationic peptides, CpG oligonucleotide, squalene-based adjuvants, preferably MF59; cytokines, such as IL-1 and IL-2; or formulated as virus-like particles or nanoparticles; or formulated as virus-like particles or nanoparticles; or combinations thereof.
   18. Vaccine according to any one of embodiments 1 to 17, wherein the vaccine is formulated for intradermal, subcutaneous (s.c.), parenteral, preferably intramuscular (i.m.), mucosal, transcutaneous or topical administration.
   19. Vaccine according to any one of embodiments 1 to 18, wherein the vaccine comprises 0.1 μg to 5 mg, preferably 0.5 to 1000 μg, more preferred 1 to 500 μg, even more preferred 5 to 300 μg, especially 10 to 200 μg, each of the DsA1 and/or DsA2 and/or PITP, and/or a fragment or derivative of DsA1 or DsA2 or PITP per dose.
   20. Vaccine according to any one of embodiments 1 to 19, wherein the DsA1 and/or DsA2 and/or PITP, and/or a fragment or derivative of DsA1 or DsA2 or PITP is provided as DNA or RNA encoding an antigen or an epitope containing fragment or derivative thereof, preferably as mRNA vaccine, especially an mRNA vaccine formulated with cationic polymers, with an mRNA molecule with the following structure: 5′UTR-signal peptide-encoded antigen or epitope-3′UTR; or as a vector-based vaccine, especially with an adenovirus vector, an adeno-associated virus vector, a vesicular stomatitis virus vector, a Newcastle Disease virus vector, an alphavirus vector, a baculovirus vector, or a retrovirus vector; and/or wherein the coding sequence on DNA level has the initial coding DNA sequence ATGGTG: especially wherein the mRNA comprises a sequence as transcribed from SEQ ID NOs: 67 to 76.
   21. Vaccine according to any one of embodiments 1 to 20, wherein the vaccine comprises 5 to 500 μg each, preferably 20 to 100 μg each, of DsA1 and/or DsA2 and/or PITP, and/or a fragment or derivative of DsA1 or DsA2 or PITP; or DNA or RNA encoding an antigen, or a fragment or a derivative thereof, per dose.
   22. Vaccine according to any one of embodiments 1 to 20, wherein the vaccine comprises less than 1 μg each of DsA1 and/or DsA2 and/or PITP, and/or a fragment or derivative of DsA1 or DsA2 or PITP per dose.
   23. Vaccine according to any one of embodiments 1 to 22, wherein the vaccine does not contain Freund's incomplete adjuvant.
   24. Vaccine according to any one of embodiments 1 to 23, wherein the vaccine contains a composite immunogen and is engineered by linking of one or more antigen(s) of P. acnes and/or one or more cross-reactive, especially cross-type-reactive, or otherwise immunologically relevant epitope(s) of P. acnes to each other, preferably wherein the immunogen comprises or consists of at least 2 (e.g. 2, 3, 4, 5, 6, 7, 8, 9, or 10) antigen(s) and/or immunologically relevant epitope(s) thereof, especially wherein the antigens and/or epitopes are cross-reactive, especially cross-type-reactive.
   25. Vaccine according to any one of embodiments 1 to 24, wherein the vaccine contains a composite immunogen comprising or consisting of at least two (e.g., 2, 3, 4, 5, 6, 7, 8, 9, or 10) antigen(s) of P. acnes and/or epitope(s) thereof linked to an adjuvant, wherein at least one antigen or epitope is immunologically relevant.
   26. Vaccine according to any one of embodiments 1 to 25, wherein the vaccine contains a composite immunogen comprising at least two antigen(s) and/or epitopes thereof of P. acnes covalently linked to each other, preferably wherein the immunogen comprises at least two antigen(s) and/or epitopes thereof further linked to an adjuvant, especially wherein at least one antigen or epitope is immunologically relevant.
   27. Vaccine according to any one of embodiments 1 to 26, wherein the vaccine contains a composite immunogen wherein one antigen or epitope thereof is linked to an adjuvant, especially wherein the at least one antigen or epitope is immunologically relevant.
   28. Vaccine according to any one of embodiments 1 to 27, wherein the vaccine comprises a variant of an antigen or of an epitope.
   29. Vaccine according to any one of embodiments 1 to 28, wherein the vaccine comprises a polypeptide with at least about 90% amino acid sequence identity, preferably at least 95%, more preferably at least 98%, more preferably at least 99% or at least 99.5%, to the amino acid sequence of a naturally occurring P. acnes antigen; or a polypeptide of a P. acnes epitope, wherein at least 1, 2, 3, 4, up to 5 point mutations are contained in the amino acid sequence compared to the naturally occurring epitope.
   30. Vaccine according to any one of embodiments 1 to 29, wherein the vaccine comprises a variant, preferably a PITP polypeptide wherein one or more amino acid residues are added, or deleted, at the N- or C-terminus, as well as within one or more internal domains, especially a variant comprising additional amino acids at the N-terminal and/or at the C-terminal end, to prolong an antigen sequence, e.g. to prolong a sequence of an epitope or epitopic region within a protein by at least one amino acid residue, preferably by less than 3 amino acids, specifically less than 5, or else less than 10 amino acids.
   31. Vaccine according to any one of embodiments 1 to 30, wherein the vaccine comprises a derivative, especially as a fusion protein, wherein a PITP fragment containing a PITP epitope is prolonged by additional amino acid residues of another polypeptide or protein, preferably by one or more immunologically relevant epitopes, especially wherein the derivative comprises a His-tag at the N- or at the C-terminus comprising at least 4, preferably at least 5, especially at least 6 histidine residues.
   32. Vaccine according to any one of embodiments 1 to 31, wherein the vaccine comprises a fragment or derivative of DsA1 or DsA2, wherein DsA1 and DsA2 comprise from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Leu repeat containing region (“PT repeat region”), and a C-terminal region (“CTR”), wherein fragment or derivative comprises or consists at least of (1) a contiguous polypeptide sequence of phenylalanine 150 (F150) to leucine 184 (L184), (2) a contiguous polypeptide sequence of phenylalanine 150 (F150) to leucine 267 (L267), or (3) a contiguous polypeptide sequence of histidine 218 (H218) to leucine 267 (L267), with the proviso that the fragment or derivative does not comprise the NSR and the CTR, said fragment or derivative optionally comprising one or more amino acid exchanges, wherein one or more amino acid residues are changed to a cysteine (Cys, C) residue to further stabilise the fragment or derivative by the ability to form disulfide bonds.
   33. Vaccine according to any one of embodiments 1 to 32, wherein the fragment or derivative has a length of at least 20, preferably at least 30, more preferred at least 60 amino acids, even more preferred a length of at least 80 amino acids, especially a length of at least 100 amino acids.
   34. Vaccine according to any one of embodiments 1 to 33, wherein the fragment or derivative has a length carrier.
   36. Vaccine according to any one of embodiments 1 to 35, wherein two or more amino acid residues in a fragment or derivative are changed to a cysteine (Cys, C) residue to further stabilise the fragment or derivative by the ability to form disulfide bonds, preferably (with respect to the DsA1 sequence (which is also applicable to the DsA2 sequence; FIG. 12A)) containing at least one of the amino acid pairs in the following group wherein the native amino acid is exchanged to cysteine: PHE150-GLY185, LEU 167-ALA199, ASN157-ALA192, LYS166-ALA199, ALA153-GLY 185. ASP164-ALA199, VAL154-ALA179, PHE150-LEU184, VAL154-ALA188, ALA153-ALA188, ALA179-GLU191. THR178-GLU191, ALA161-ALA195, ILE175-ALA195. ILE175-GLU191, LEU158-ALA176, ASN157-ALA188, ASN157-ALA179, ASP164-GLU196, VAL154-LEU180. ALA153-LYS189. ASN157-LYS189, ALA161-ALA192, LEU 167-ALA198, LEU158-ALA179, ALA160-ALA192, LYS165-ALA199, LYS166-GLN200: VAL205-ALA260, ILE 232-ARG266, SER235-ILE263, SER235-ARG262, ILE232-LEU267, PRO236-ILE263, ALA231-ARG266, ASN239-ILE259, ILE232-ILE263, VAL209-PRO236, ALA201-ALA256, SER235-ILE259, VAL228-ARG266, ALA202-ALA256, ALA201-VAL257, GLY206-VAL243, GLY208-ILE264, GLY208-ILE263, VAL205-ASN239, LEU238-ILE259, GLY208-ALA260, ALA201-LYS253, ALA202-VAL243, LEU204-ALA260, VAL205-ALA256, VAL205-ILE259, SER235-ARG266, ASN239-ALA260, VAL209-ILE263, or VAL209-ASN239.
   37. Vaccine according to any one of embodiments 1 to 36, wherein a cysteine in the DsA1, DsA2 or PITP or fragment or derivative thereof is exchanged, preferably wherein the cysteine is exchanged with serine, proline, alanine, threonine, asparagine, glutamine, valine, isoleucine, leucine, phenylalanine, tyrosine and glycine, more preferred with serine, proline, alanine, threonine, asparagine, glutamine, valine, isoleucine, leucine, especially with serine, proline, alanine, threonine, asparagine, and glutamine.
   38. Vaccine according to any one of embodiments 1 to 38, wherein the fragment or derivative has a length of length of 35 to 350 amino acids, preferably of 40 to 300 amino acids, more preferred of 50 to 250 amino acid residues, more preferred of 60 to 200 amino acids, more preferred of 70 to 150 amino acids, especially of 90 to 130 amino acid residues.
   39. Vaccine according to any one of embodiments 1 to 37, wherein the derivative comprises at least one polypeptide stretch of DsA1 and at least one polypeptide stretch of DsA2, preferably wherein the derivative comprises at least CSD1, CSD2 or CSD3 of DsA1 and at least CSD1, CSD2 or CSD3 of DsA2.
   40. Vaccine according to any one of embodiments 1 to 39, wherein the derivative comprises at least one fragment comprising at least CSD1, CSD2 or CSD3 of DsA1 and at least one fragment comprising at least CSD1, CSD2 or CSD3 of DsA2 and wherein the at least two fragments are interconnected by
an SR1 or SR2 of DsA1 or DsA2 adjacent to the at least CSD1, CSD2 or CSD3 of DsA1 and at least CSD1, CSD2 or CSD3 of DsA2; or
an amino acid sequence consisting of an N-terminal part of an SR1 or SR2 of DsA1 or DsA2, adjacent to the at least CSD1, CSD2 or CSD3 of DsA1 and at least CSD1, CSD2 or CSD3 of DsA2, and a C-terminal part of a different SR1 or SR2 of DsA1 or DsA2, adjacent to the at least CSD1, CSD2 or CSD3 of DsA1 and at least CSD1, CSD2 or CSD3 of DsA2, wherein the total number of amino acid residues the amino acid sequence consisting of the of the N- and C-terminal part is identical to the SR1 or SR2 of DsA1 or DsA2.
   41. Vaccine according to any one of embodiments 1 to 40, wherein the derivative comprises at least one fragment comprising at least CSD1, CSD2 or CSD3 of DsA1 and at least one fragment comprising at least CSD1, CSD2 or CSD3 of DsA2, wherein the fragment of DsA1 and DsA2 has independently a length of at least 20 amino acid residues, preferably at least 30, more preferred at least 40, especially at least 50, amino acid residues
   42. PITP polypeptide, and/or a fragment and/or a derivative of PITP according to any one of embodiments 1 to 41 for use in a therapeutic and prophylactic treatment, preferably for use in the treatment or prevention of P. acnes-associated infections, especially selected from the group consisting of acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, artherosclerosis, prostethic cancer and a medical implant biofilm infection by P. acnes.
   43. PITP polypeptide, and/or a fragment and/or a derivative of PITP according to any one of embodiments 1 to 41 for use in the treatment or prevention of P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes.
   44. PITP polypeptide, and/or a fragment and/or a derivative of PITP for use according to embodiment 42 or 43, wherein the human patient is suffering from P. acnes-associated infections and pathological conditions associated with at least two P. acnes MLST phylotypes, and/or at least two different CC types of P. acnes and/or at least two ribotypes of P. acnes, as determined by a surface binding measurement employing flow cytometry and in a bactericidal killing assay using immune serum raised against such vaccine antigen.
   45. PITP polypeptide, and/or a fragment and/or a derivative of PITP for use according to any one of embodiments 42 to 44, wherein the human patient is suffering from P. acnes-associated infections and pathological conditions associated with at least two or three genetically different types of P. acnes strains, preferably against at least one Type I and at least one Type II or III strains; or from at least one Type II and at least one Type I or III strains; or from at least one Type I and at least one Type I or II strains; or from at least one Type I, at least one Type II, and at least one Type III strain, especially Types IA1, IA2, IB, II and III.
   46. PITP polypeptide, and/or a fragment and/or a derivative of PITP for use according to any one of embodiments 42 to 45, wherein the human patient is suffering from P. acnes-associated infections and pathological conditions associated with two or more of P. acnes strains selected from the group consisting of Type IA1, Type IA2, Type IB, Type IC, Type II and Type III strains, or Type I-Ia, I-lb, I-2 and II, as defined according to the MLST typing schemes, especially Types IA1, IA2, IB, II and III.
   47. PITP polypeptide, and/or a fragment and/or a derivative of PITP for use according to any one of embodiments 42 to 46, wherein the human patient is suffering from P. acnes-associated infections and pathological conditions associated with two or more of P. acnes strains selected from the group of the ribotypes as determined according to 16S ribosomal sequence differences.
   48. PITP polypeptide, and/or a fragment and/or a derivative of PITP for use according to any one of embodiments 42 to 47, wherein the human patient is suffering from P. acnes-associated infections and pathological conditions associated with two or more phylotypes IA1, IA2, IB1, IB2, IB3, IC, II and III determined by the analysis of a single locus (SLST), especially Types IA1, IA2, IB, II and III.
   49. PITP polypeptide, and/or a fragment and/or a derivative of PITP for use according to any one of embodiments 42 to 48, wherein the human patient is suffering from P. acnes-associated infections and pathological conditions associated with at least two different phylogenetic types, more preferably three, even more preferably four, five or six phylogenetic types of the following group of strains: NCTC737, KPA171202 (DSMZ, German Collection of Microorganisms and Cell Cultures, Braunschweig. Germany), SK137, HL005PA1, HL005PA4, HL013PA1, HL030PA1, HL043PA1, HL053PA1, HL053PA2, HL050PA1, HL050PA2, HL060PA1, HL110PA4 (BET, Biodefense and Emerging infections Research Resources Repository, Manassas, VA), P.acn31 and Asn12 (McDowell et al. 2012) and IAI 008, IAI031, IAI034, IAI035. IAI038, IAI040, IAI042, IAI045, IAI041 (Charit. Berlin, Pro-Implant foundation).
   50. PITP polypeptide, and/or a fragment and/or a derivative of PITP for use according to any one of embodiments 42 to 49 for use in protection against infection with P. acnes.
   51. PITP polypeptide, and/or a fragment and/or a derivative of PITP for use according to any one of embodiments 42 to 50 for use in prevention or reducing development of a P. acnes-associated indication.
   52. PITP polypeptide, and/or a fragment and/or a derivative of PITP for use according to any one of embodiments 42 to 51, wherein the vaccine repeatedly administered to a human patient, preferably by at least 2 or 3 administrations, or at least 4, 5, or even more repeated administrations.
   53. PITP polypeptide, and/or a fragment and/or a derivative of PITP for use according to any one of embodiments 42 to 52, wherein the vaccine is used in one or more treatment cycles, each involving at least two, or three or four consecutive administrations within a period of 1 year in intervals of at least 1 or 2 weeks, especially wherein the treatment cycles are repeated at least any of 1×, 2×, 3×, or 4×, even more times, e.g. within a period of 5 years.
   54. PITP polypeptide, and/or a fragment and/or a derivative of PITP for use according to any one of embodiments 42 to 53, wherein the vaccine provokes an immune response, including the production of antibodies and/or cytokines and/or the activation of cytotoxic T cells, B-cells, antigen presenting cells, helper T cells, dendritic cells and/or other cellular responses in an individual upon administration.
   55. PITP polypeptide, and/or a fragment and/or a derivative of PITP for use according to any one of embodiments 42 to 54, wherein the vaccine is administered to a patient with a skin or other affected organ or tissue condition in which the strains from any of Type I, II, or III P. acnes, or a combination of at least two phylotypes of Type I, If, and III, or of at least two ribotypes of P. acnes, especially Types IA1, IA2, IB, II and III, are involved as pathogenic factors, or to a healthy individual that may be susceptible to an infection by any of the P. acnes strains.
   56. PITP polypeptide, and/or a fragment and/or a derivative of PITP for use according to any one of embodiments 42 to 55, wherein the vaccine is administered to a patient by intradermal, transdermal or a subcutaneous administration, preferably by intradermal injection applicators, microneedles, transdermal laser devices, topical, skin patch or other suitable skin-adapted application device.
   57. PITP polypeptide, and/or a fragment and/or a derivative of PITP for use according to any one of embodiments 42 to 55, wherein the vaccine is administered by oral or parenteral administration such as intramuscular, intranodal, intranasal, mucosal, intrapulmonary, vaginal, rectal or other suitable routes.
   58. Fragment or derivative of DsA1, DsA2 or PITP as defined in any one of embodiments 1 to 57, preferably a fragment or derivative according to SEQ ID NOs: 5 to 7, 9 to 0.12, 14 to 20, and 29 to 50.
   59. Pharmaceutical composition comprising a fragment or derivative according to embodiment 58 or a PITP polypeptide.
   60. Pharmaceutical composition according to embodiment 59, further comprising a pharmaceutically acceptable carrier.
   61. A method for producing a vaccine, wherein a PITP polypeptide and/or a fragment and/or or a derivative of PITP as defined in any one embodiments 1 to 41 is mixed with a pharmaceutically acceptable excipient or carrier and, optionally, finished into a pharmaceutically administrable vaccine dose, preferably in a dosage unit suitable for administration to an individual, especially a human individual.
   62. Vaccine, composition, method or PITP polypeptide according to any one of embodiments 1 to 61, wherein the PITP and/or DsA1 and/or DsA2, and/or a fragment or derivative of PITP or DsA1 or DsA2 is provided as DNA or RNA encoding an antigen or an epitope containing fragment or derivative thereof, preferably as mRNA vaccine, especially an mRNA vaccine formulated with cationic polymers, with an mRNA molecule with the following structure: 5′UTR-signal peptide-encoded antigen or epitope-3′UTR; or as a vector-based vaccine, especially with an adenovirus vector, an adeno-associated virus vector, a vesicular stomatitis virus vector, a Newcastle Disease virus vector, an alphavirus vector, a baculovirus vector, or a retrovirus vector; and/or wherein the coding sequence on DNA level has the initial coding DNA sequence ATGGTG; especially wherein the mRNA comprises a sequence as transcribed from SEQ ID NOs: 67 to 76.
   63. Vaccine, composition, method or PITP polypeptide according to any one of embodiments 1 to 62 further comprising a DsA1 and/or DsA2 derivative, wherein the DsA1 and/or DsA2 derivative preferably comprises one or more, preferably one, two or three, especially one, of the following amino acid exchanges: DsA1: A301Y, G206L, G206F, A192L, G206R, G206W, G206Q, G206K, A301F, A301L, G206I, A301W, A301I, G206E, G206M, G208L, G206V, A192M. A256L, A1921. A68I, G206N, A68V, I259L, A161L, V205I, 0206T, G206H, A68L, G206Y, A256I, G206A, 68M, A192F, G110A, N239L, G206C, A301V, E305L, A93I, A93L, S223A, E305I, E305V, E305A, K120V, K120L, N157I, K120C, E305C, E305M, N239I, K120I, R248C, N239V, E191I, E305F, S78I, K120M, E191L, R248L, H146V, E191V, H1461, S78L, S78V, N157V, H146C, K120F, R248I, R248M, D164L, P236V, P236L, H146L, H146F, D164I, H146M, P86L, R2480, N239C, N157C, E305T, E191C, S78C, R248F, G221L, E305G, P236F, T171L, P86V, R248S, N157L, K120A, H146A, P861, N157F, S235A, R248A, E191M, N157M. A124I, P236A S78F, P236C, S78M, A161I, N239M, T171I, E191F, S235V, P86F, H146G, S223I, E305S, D164F, E305Y, P236M, S223L, N239F, H146Y, P236I, G221V, D164C, K120G, D164M, S235I, R248V, A124V, S235L, S223V, S147L, S235C, P86M, G208I, D164A, S1471, N239A, K120T, T117I, G110L, R248Y, P182I, A198L, A124L. A215L, A127L. A66L, P86C, G221F, R248T, T171F, P182V, S147V, P271V, T171V, G110V, P271A, E191A, A149L, K120Y, G114I, K120S, S235F, A201I, G131V, A201V, R248N, A66V, A202I, G221C, G114V, A229L, H146T, A124F, A127F, T117V, S223C, A215F, A1271, P182C, A215V, G162L, G162I, G208F, A201L, A161F, N157A, A198V, E191Y, A161V, S223F, G221M, R248W, G131C, A202V, N157T, S147F, N157Y, G114A, T171C, H146W. S78T, A149I, G250L, N239T, A149V, G208C, K120P, G250V, S235G, S223M, D164V, N239G, T171M, E191G, S78A, D164Y, P271I, G110F, G110C, D164W, P236Y, A124C, A66F, S147M, G75L, E305N, S147C, G221I, A127M, P182L, G221A, G110M, A66C, E305Q, P271F, A215M, P236W, K120W, H146P, A161C, A2291, S78G, G114M, P86A, E305W, V154L, G185V, G75I, S223Y, G75V, R248Q, T277V, E91S, A229F, T277G, G1311, N239S, P271C, V154I, A66M, D164G, G208A, P236T, R248D, D164T, A127C, A215I, A161M, P86W, A201F, C53L, T117F, G131A, T117C, A318L, F150L, G208M, P271M, A149C, G208V, G114C, G162F, S223W, A198I, N157G, A202L, P236G, G185I. A198F, A149M, P86Y, E191W, N157W, I263L, S235T, E305P, E305R, A201C, A201M, F150I, G96I, G114F, G221Y, A199L, D164S, A124M, A179I, A202F, H146S, A231L, C53I, I263V, G162M. K120Q, N239Y, G96L, G75C, N157S, T277C, T117A, A199V, G162C, P271Y, G250C, A127Y, A661, T117L, G185C, A229C, R248P, A1441, T117M, P86T, A260L, G185A, E191Q, G131L, T277I, G250I, G131M, T277A, A127V, F150V, A318I, S78W, S147Y, A198M, P86G, A215C, A701, S235M, A179V, A202M, A144V, A202C, G208W, T171Y, S147W. G208Y, G250F, A195L. K120H, A127W. A66Y, S223G, K120N, S78Y, E191N, A198C, P271G, V154F, A70V, H146N, A229M, A134L, P86S, S78Q, A199I, G75M, A68C, E191T, A89L, G96A, D164P, C53V, S78P, G185L, A149G, A149F, P182A;
   DsA2: A61L, A161I, A126I, A126V, A126L, S175G, G190H, A161T, S183T, R336I, A126F, A161F, A329L, A161V, S283L, A205L, G109A, A329I, G265F, A161W, A232I, S175W, R336L, A300I, K349I, A161M, A300L, G109L, G109I, A246L, S183M. A223L, E235V, G252L, A329M, A126W, S183L, A259L, A329V, A329F, S183I, A242L, G109F, R336V, K117I, R336C, K117C, K349V, R336A, R336F, K117L, R336M, E235I, R310A, K349L, K117V, R310V, K349C, N201I, R310I, R310C, K349F, K349M, K117M, R292C, N201V, R336G, R292L, K117F, E235C, P130L, E235A, D208L, P280L, E235L, R292M, R336T, P280V, R292I, N201L, E235M, P130I, K117A, K349A, P130V, R292G, R310F, R336S, R336Y, N201C, K117G, R310L, R310M, R310G, R336W, D208V, R292F, P130F, K349Y, K117T, R310T, P280C, R292S, N201F, R310S, T215L, N201M, S279C, R292A, S279L, P280F, D208C, S175V, A126M, P280A, S279A, P130C, S279V, E235F, P186C, P130M, K349T, A126C, K349G, N320V, K117S, N320I, S283V, A168I, N320L, S175I, E235Y, P186V, P280I, P186A, P280M, G265L, D208M, P186L, K117Y, S283I, A205I, D208F, A168L, T215I, R292V, A133L, R336P, R310Y, A168V, S279F, S183V, A164I, S279I, S175L, D208A, K349S, E235T, S175C, A122I, D208I, A126Y, A123L, K117P, N320C, R292T, S283C, E235W, G252I, T215V, R292Y, R336Q, S175A, A123I, E235G, A205V, A171V, A133V, A245I, S183C, P226V, R310P, G181I, P226I, A168C, A246I, A171L, N201A, S175M, G158I, A164L, T215C, A164C, P280W, A133C, G1191, T215F, A245V, N320M, A122V, A123V, G206L, N320A, G265V, A122L, A193L, S91V, R310W, G252F, G181V, E235S, R292N, P130W, G119V, G265C, S279M, A259V, A259F, A168F, A168M, A246V, P226C, R292W, G158V, P130Y, G206V, P186G, C97L, N201Y, A171C, G265M, G140L, K117N, G140I, D208G, A193V, G181F, K349W, S279G, A245L, A205F, G158A, A193I, K117W, A122C, R310D, G294V, P186T, S175F, S191C, N201T, K349Q, R310N, A242V, T215M, D208Y, T321C, G252C, 1276L, A259M, A171F, N201G, G119F, D208T, P186M, G229V, G294L, S191I, P186S, G119C, G2291, P130A, A205C, A171M, T321V, A193C, F268L, A133M, A123F, G357A, G109V, K117Q, N320G, R310Q, T321G, S283M, P186Y, G229A, R336D, N201S, S183F, F194L, F268V, A245F, G265A, G252A, R336H, A259I, A133I, A123C, P130T, G113L, D208S, P280T, F268I, G181C, P280Y, R336E, P280G, G158C, N320Y, G140V, K349P, R292D, D208W, N320F, G265I, R336N, A164M, G206M, F268C, G158M, R292P, S191L, G158F, G252M, A245C, G229C, E235Q, G252V. A205M, G206F, R292Q, G181M, A126G, A243L, K349N, S283A, A246M, S279T, P186I, A246F, A246C, A2231, C97I, A164V, A245M, A171I, G357I, A126T, S283F, A123M, F194I, K117D, P226L, P130S, G140F, I276F, F194V, A243V, P130G, G181L, G229F, A259C, G113I, K117H, N320S, G119M, A239L, G206C, A114I, E235N, K349D, A242I, A161C, G294C, G119L, A126S, T321L, A193M, A223V, A122M, G113C, A242C, N201W, R292H, A275L, A242M, G357V, P186F, G294I, D208P, A259W, A243I, R310E, G140C, A242F, G265W, S183A, G109C, G113F, S191F, G265Y, S94D, E95K, A343K, A350K, F176D, N196D, H147D, H146D, H147E. H146E, T91E. R250E, T91D, R251E, R148D, H254E, A170K, R250K, R251K, H254D, A170R, H146K, H147K, K346E, T90D, R250D, T90E, A138K, R251D, H262K, A169K, H146R, I102E, T90K, A271K. K346R, H306E, D124E, K326E, A169R, D124R.
   64. Vaccine, composition, method or PITP polypeptide according to any one of embodiments 1 to 63, comprising a PITP derivative, wherein the PITP derivative comprises one or more, preferably one, two or three, especially one, of the following amino acid exchanges:
   T392F, F352L, T392I, M303F, A276Y, S365I, T392L, M303W, A276L, T392W, K363T, A361V, F286Y, A361I, K363F, M303Y, T392V, A361L, S365V, A270V, A276M, S365L, K363V, M312L, T252L, M387L, Y297F, A276F, G353T, A276W, K363S, T252R, L314I, T252I, A361F, L295I, L255I, A276I, K363L, S318W, V354L, K363I, A361T, S318I, A361M, G353L, T392Y, S365A, A270I, A361W, T252M, K363A, K363W, L319I, G353D, A276V, G353N, G353F, T252V, G353S, S318V, L255V, G364I, L394V, K363M, S318F, L390I, G353I, S335Y, G364L, S365M, T252Q, S365F, L330A, T252F, S318M, K363Y, G353V, S365G, S365W, K363G, T392M, V354F, A276T, G364W, K363C, P317I, S335C, P317L, P317F, G269C, S335A, P317V, G268C, S334V, S335V, G269V, S334C, A361C, G268I, G269F, G268L, S334I, G268V, G269L, S335G, S334A, G268F, S335T, P317C, P317M, S335L, A361Y, 0269I, S335I, G268Y, K363P, G268W, S335M, K363H, F286I, G268M, G269M, G269W, G268A, S334G, S334L, G268P, G268T, G293F, F286L, T252C, S335N, T392C, K363N, S334F, M303I, K363Q, A376L, G364V, W245I, G269S, Y297C, S335Q, G358V, W245L, A376I, S334M, G358L, T252A, S318C, S335H, Y297L, S365C, M312I, G373L, Y297I, G358I, S335F, G268N, M303L, G269T, Y297V, P317A, G268Q, G268H, G293L, S334T, G305C, S318L, S335D, S335P, G364C, W245V, W245F, P317Y, T392A, F286V, S335W, A376V, G358C, K363D, M312F, G269N, S335R, A378I, A378C, P317W, G268K, G268R, G268D, A376C, G373V, M312V, M303V, G353C, S318A, G364F, G241C, G268E, G379I, G269P, F286C, L330F, G284V, W245C, A378V, L330I, G293I, G373C, G379L, S318Y, G358F, L394I, L330V, G373I, F352I, A361G, S335K, G246C, M387F, A378L, G241I, V354C, M3871, 0269A, G308C, F352C, A276C, G293C, G269Q, K363R, G379V, F286W, W245M, G246V, A270C, G246L, M303C, G293V, G305A. Y297A, G345I, G241V; N388D, R248K. S336D, T372E, T249E, E316D, D348E, R382E, R382K, K265E, H327K, D338E, K333R, P386E, S336E, K341D, K341E, K341R, E385D, G268K, G268R, G268D, K340R, G268E, K333D, K368R, G353D; H306I+S335R, H306L+S335R, H306I+S335K, H306C+S335R, H306C+S335R, H306I+S335K. H306L+S335K, H306L+S335K, H306L+S335R, H306V+S335R, H306I+S335R, H306V+S335R, H306C+S335K, H306F+S335K, H306V+S335K, H306V+S335K, H306F+S335R, H306F+S335R, H306W+S335R, H306F+S335K, H306M+S335R, H306W+S335K, H306W+S335R, H306M+S335K, H306A+S335R, H306A+S335R, H306W+S335K, H306A+S335K, H306Y+S335R, H306Y+S335R, H306Y+S335K, H306T+S335R, H306Y+S335K, H306T+S335R, H306T+S335K, H306S+S335R, H306T+S335K, H306S+S335K, S334V+S335R, H306S+S335K. S334V+S335K, S334I+S335R, S334C+S335R, H306P+S335R, M312I+S335R, S334I+S335K, S334V+S335R, S334V+S335K, H306P+S335K, S334C+S335R, S334A+S335R, M312F+S335R, M312L+S335R, S334C+S335K, H306R+Y381L, M312L+S335R, S250L+S335R, H306Q+S335R, H306N+S335K, S334C+S335K, S334A+S335K, H306P+S335K, H306Q+S335R, M312V+S335R, M312I+S335K, S334I+S335K. M312I+S335R, M312L+S335K, H306N+S335R, S334L+S335R, S334A+S335K, M312L+S335K, S2501+S335R, H306N+S335R, S250V+S335R, H306N+S335K, M312F+S335K, S250V+S335R, S250A+S335R, S334M+S335R, M312V+S335R, S250A+S335K. M312F+S335K, S250A+S335K. S250L+S335R, S334G+S335R, S250C+S335R, S250C+S335R, H306Q+S335K, S334G+S335R, Y254F+S335R, S334L+S335R, S2501+S335R, I309C+S335R, S2501+S335K, Y254C+S335R, S250L+S335K, M312V+S335K, S334G+S335K, S250L+H306R, S250V+H306R, H306R+Y381I, M312V+S335K, S2501+H306R, M312C+S335R, Y254C+S335R, S250V+S335K, Y254W+S335R, S334G+S335K, S250L+H306R, S250L+S335K, Y254W+S335R, S250V+S335K, S334L+S335K, I309C+S335R, I309C+S335K, Y254F+S335R, S250A+S335R, S250C+S335K, S2501+H306R. S250A+H306R, S250C+S335K, H306Q+S335K, Y254C+S335K, S334L+S335K, S250A+H306R, I309C+S335K, Y254W+S335K, Y254C+S335K, Y254I+S335R, H306R+Y381C, S250M+S335K, M312C+S335K, M312C+S335K, S250V+H306R, S250M+S335R, Y254V+S335R, Y254I+S335K, S250W+S335R, I259C+S335R, S334M+S335R, Y254A+S335R, Y254V+S335R. S250M+S335K, F251L+S35R, Y2541+H306R, I259C+S335K, Y254I+S335R, I259C+S335K, S250Y+S335K, Y254F+S335K, S250M+S335R, Y254F+H306R, Y254V+S335K, Y254V+H306R, S334T+S335K, S334Y+S335R, Y254A+S335R, S334M+S335K, Y2541+H306R, Y254M+S335K, Y254A+S335K, S334F+S335R, S334M+S335K, F251L+H306R, S334T+S335R, Y254I+S335K, Y254A+S335K, S334T+S335K, S250Y+H306R, Y254A+H306R, Y254M+S335K, F251L+S335K, I259C+S335R, F251L+H306R, Y254M+H306R, S334P+S335R, S334P+S335R, S334P+S335K, S334F+S335R, Y254M+S335R, H306I+S335C, H306L+S335C, H306L+S335C, H306I+S335C, H306V+S335C, H306C+S335C, H306I+S334V, H306V+S335C, H306C+S335C, H306I+S334V, H306L+S335V, H306M+S335C, H306I+S335G, H306I+S335G, H306I+S335A, H306I+S335A, H306F+S335C, H306F+S335C, H306L+S335V, H306I+S335T, H306L+S335A, H306C+S335A, H306I+S335V, H306I+S335V, H306L+S334V, H306L+S335A, H306C+S335V, H306L+S334C, H306I+S335I, H306I+S334A, H306L+S334C, H306L+S334V, H306F+S334V, H306C+S335A, H306F+S335V, H306C+S334V, H306C+S334V, H306I+S335L, H306C+S335V, H306L+S334I, H306M+S335C, H306I+S334C, H306I+S335I, H306W+S335C, H306V+S335A, H306I+S334C, H306I+S334I, H306L+S335I, H306V+S335A, H306C+S334C, H306L+S335G, H306C+S334C, H306L+S335T, H306I+S334I, H306V+S334V, H306I+S335L, H306M+S335V, H306V+S335V, H306V+S335V, H306I+S335M, H306L+S335T, H306V+S335I, H306C+S335G, H306L+S335L, H306C+S334I, H306L+S335Y, H306L+S335I, H306V+S334V, H306C+S335G, H306I+S334A, H306L+S334A, H306C+S335I, H306F+S335V, H306I+S335T, H306L+S335Y, H306C+S334I, H306L+S334I, H306V+S3350, H306V+S335G, H306A+S335C, H306A+S335C, H306M+S335A, H306C+S334A, H306V+S335I, H306L+S335G, H306L+S334A, H306C+S334A, H306I+S335Y, H306I+S335Y, H306W+S335C, H306F+S335A, H306F+S335A, H306F+S335I, H306I+S335N, H306L+S335L, H306I+S335M, H306I+S335N, H306V+S334I, H306V+S334C, H306V+S335L, H306C+S335L, H306C+S335L, H306I+A376L, H306F+S335T, H306L+S335M, H306F+S334V, S334V+S335C, S335C+Y381L, S335C+A376I, S334I+S335C, S334V+S335C, M312I+S335C, M312I+S335C, S335C+A376L, S334I+S335C, S335C+A376L, S335C+A378C, S335C+A376C, S335C+A376V, S334A+S335C, S335C+A378C, S334A+S335C, S335C+A376I, S334V+S335A, S335A+A376L, S335C+A376C, S335C+A376V, S335C+A378L, S334V+S335A, S335A+A376I, S334V+A376L, S334I+S335V, S335C+A378I, M312F+S335C, M312F+S335C, S334I+S335A, S335C+A378V, S335V+A376L, S334V+S335L, S335C+A378I, S334C+S335V, S334V+A376I, S334V+A376I, S335V+A376I, M312I+S334V, S334V+S335I, S335C+Y381I, M312V+S335C, S335C+A378L, S335A+Y381L, S334C+A376C, S334V+S335I, S335C+Y381V, M312L+S335C, S335C+A378V, S335V+A376L, S334I+S335V, S335C+Y381C, S335V+A376I, S334C+A376L, S334C+Y381L, M312V+S335C, S334V+S335L, S334C+A376I, S334C+A376C, S334V+Y381L, M312L+S335C, S335V+A376V, S334I+A376I, S335V+A376V, S335V+Y381L, Y254F+S335C, L310F+S335C, S335I+A376I, S334V+A376V, M312I+S335V, S334C+S335A, S334I+A376L, M312F+S335V, S335C+Y381A, L310F+S335C, S334G+S335C, S334G+S335C, S334V+A376V, S335C+A376M, S335C+A376M. S334A+S335I, S334V+A376L, M312C+S335C, S334C+S335A, S335A+A376V, S334A+S335V, S334A+S335V, Y254F+S335C, S335A+A376L, S335A+Y3811, S335A+A376I, S334L+S335C, S335A+A376V, M312C+S335C, D152F, D152L, D152I, D152V, D152M, D152W, D152Y, D152S, D152A, D152T, D152G, D152N, D152Q, D152P, D152H, D152K, D152R, D152C, D155Q, D155L, D155F, D155I, D155M, D155W, D155V, D155Y, D155A, D155N, D155T, D155S, D155P, D155G, D155H, D155R, D155K, D155C, D156L, D156F, D156I, D156W, D156V, D156A, D156Y, D156M, D156G, D156S, D156T, D156Q, D156N, D156P, D156H, D156K, D156R, D156C, H146R, H146K. H153R, H153K, Y63A, H146A, H153A, F74A, L141A, F81A, P72A, K144A, T143A, Y75A, W98A, D156A, R158A, S250W, S250F, S250D, S250E, S250K, S250R, F251 A, Y254A, I259W, I259F, I259D, I259E, I259R, I259K, H306A, 1309W, I309F, I309E, I309D, I309R, I309K, L310W, L310F, L310D, L310E, L310R, L310K, M312W, M312F, M312D, M312E, M312R. M312K, S334W, S334F, S334D, S334E. S334R, S334K, S335W, S335F, S335D, S335E, S335K, S335R, M337W, M337F, M337D, M337E, M337K, M337R, A376W, A376F, A376E, A376D, A376R, A376K, F377, A378W, A378F, A378D, A378E, A378K, A378R, F380A, Y381A, D348I, D348L, D348M, D348F, D348V, D348Y, D348A, D348W, D348T, D3480, D348P, D348H, D348S, D348R, D348K, D348E. D348C, H306R, H306D, H306E, H306K, H307D, H307R, H307E, H307K, H327R, H327K, H327E, H327D, H302E, H302K, H302R, H302D, H215K, H215R, H215E, H215D, H223K, H223R, H223E, H223D, H403K, H403R, H403E, H403D.
   65. Vaccine, composition, method or PITP polypeptide according to any one of embodiments 1 to 64, comprising a polypeptide with at least one P. acnes epitope of the polypeptide according to SEQ ID NO: 49, wherein the polypeptide with at least one P. acnes epitope of the polypeptide according to SEQ ID NO: 49 preferably comprises one or more, preferably one, two or three, especially one, of the following amino acid exchanges compared to SEQ ID NO: 49:
   A274Y, S26C, S26L, A274F, S261, 0119H, A274L, A274W, S26V, S26M, S212L, T108I, A134L, S26F, N130V, A229L, A274I, G194F, S26W, A291F, G181I, A229I, E278I, A175L, G181L, A152L, A274V, N130L, E278V, K46I, E278L, E278A, K46C, K46L, K93L, K93V, E164I, E278C, E164V, E278M, N130I, K46V, R221C, K93C, K46M, S51L, K93I, E278F, K93M, E164A, S51I, R221L, P209L, K46F, S51V, E164C, R221I, E164M, K93F, D137L, P209V, E164L, P59L, R221F, N130C, S51C, R2210, E278T, K46G, P59V, K46A, N130M, E278G, D137V, K46T, P209C, P591, P209I, T144L, N301F, E164F, S51F, R221M, P209F, S51M, R221S, P209A, K93A, P209M, E164Y, D137C, R221A, P115C, P59F, S208A, K46S, P115V, A1341, P115A, S212V, N249L, E278S, D137F, D137M, P115L, S212I, R221V, N249V, D1371. E278Y, G194L. P59M, R221T, A188L, N249I, T144I, E164T, S208C, S208V, D137A, K46Y, K93G, G87I, E164G, K46P, P59C, A97V, S212C, 083L, K93T, N249C, A134V, 083V, T108V, T112L, T144V, A100L, G87V, E164W, E164S, A171L. A97L, A188F, K93W, T901, A174I, K93Y, A111L, A971, N130A, G48I, E278W, R221Y, G135L, T112V, F197V, T144F, K93S, G194V, G87A, A188V, T144C, K46N, P115M, A175I, N130T, S51A, G194C, G48L, G104V, A174V, G158V, T90V, R221N, A202L, A39L, A134F, D137Y, G158I, A174L, P155V, A171V, S51T, F197I, T112C, D137W, G135V, D137G, P115G, T250C, S208L, A100I, T250L, T144M, T108C, P115F, G69L, T112F, G181F, A100F, S208I, G223V, T250V, N249M, A111V, N249Y, A111F, A188M, A111I, P209Y, A175V, P155I, N130G, S120V, A111C, A100C, A97F, G194M, G69I, E278Q, S208G, G181C, G87F, G48V, A97C, I205L, G194I, D137T, S51G, F197C, G223L, N249A, S51Y, P115S, A202I, P155C, S26A, G83M, S120C, D137S, E278N, G83F, G158C, P59Y, G87C, G87M, N130S, P115I, A202F, A100M, G83C, S212M, A39V, A188I, F197L, G181M, A134C, P115T, A174F, K93P, P209W, P209G, P59A, A39C, G104I, T250G, S208F, R221Q, 0181A, G104C, T90C, K46Q, P209T, K46W, A171I, G158A. A134M, G48C, G194A, T90A, T90M, N130Y, N249F, A172L, A152V, A152I, P59W, R221P, G158F, T112M, G135M, G181V, G104L, S208T, N249W, A97M, G135F, K93Q, G42L, A111M, G104A, E278P, T90L, R221D. A175C, A188C, A291L, S212A, F123L, T250I, A174C, E164Q, P155L, A174M, G158M, A172V, E164N, A168L, T90F, A39F, G42V, I205F, A39M, N249G, A175F, G135C, A171F, S212F, K46D, A100Y, 0421, P59G, 069A, E164P, G158L, G42C, A171C, T250M, T108L, K93N, A171M, G223I, A175M, T108M, A1721, S51P, A291I, A111Y, G20V, S120I, P59T, A58I, G223C, S208M, G48M, K46H, A62L, A43I, T250A, N130W, G194Y, R221H, D137P, N249S, G104F, A100V, F123I, A172C, T112I, G69F, P115Y, P59S, A100W, G104M, G69C, R221W, K93H, A204L, G20L, G223F, A168I, D28K, E275K, K77R, D17R, H76D, H75D, H76E, K77D, H75E, P294D, S293D, S293E, D286E, P294E, S292E, D285E, H76R, K77E, K289R, P295D, D285K, R179E, H75R, P295E, P294R, P294K, D286K, D285R, S293R, S293K, R180D, R180K, H183E, R179K, K289E, R180E, A98K, R179D, A99K, S292D, D286R, K290D, H75K, A99E, N256E, K290E, K290R, R239D, A199K, A200K, K255E. H183D. A99R, H235E, A98R, D102K. and A200R.
   66. Vaccine, composition, method or DsA1 and/or DsA2 derivative according to any one of embodiments 1 to 63 wherein the DsA1 domains are defined as follows: NSR: from S29 to I48. CSD1 from I49 to L130, SR1 from G131 to S147, CSD2 from A148 to L267. SR2 from A268 to T277, CSD3 from A278 to K323, the PT repeat region from P324 to T361, and CTR from S362 to F405 in the sequence of Q6A5X9 or any other functional DsA1 sequence comprising all these domains, especially the DsA1 sequences of FIG. 12 comprising all these domains;
   and/or wherein the DsA2 domains are defined as follows: NSR: from A72 to K92, CSD1 from 193 to L174, SR1 from S175 to S191, CSD2 from A192 to L311, SR2 from A312 to T321, CSD3 from A322 to E366, the PT repeat region from P367 to T420, and CTR from H421 to A463 in the sequence of Q6A5P9 or any other functional DsA2 sequence comprising all these domains, especially the DsA2 sequences of FIG. 12 comprising all these domains;
   and/or wherein the PITP domains are defined as follows: ENFD from A32 to R164; SR1 from E165 to K237, HbD from V238 to L396, SR2 (including C-terminal LPXT(G) motif (i.e. including LPXT, but not the G)) from S397 to T430, and hLAR from G431 to 1467 in the sequence of Q6A9N1 or any other functional PITP sequence comprising all these domains, especially the PITP sequences of FIG. 12 comprising all these domains.
   67. Pharmaceutical composition according to any one of claims 1 to 64 comprising in a liquid formulation or in a dry or frozen formulation which is reconstitutable to a liquid formulation: 0.01 mg/ml to 50 mg/ml, preferably 0.05 to 5 mg/ml, especially 0.1 to 1 mg/ml, polypeptide as defined above comprising at least one P. acnes epitope, 0.1 to 100 mM, preferably 0.5 to 50 mM, especially 0.1 to 10 mM, NaH₂PO₄, 0.1 to 100 mM, preferably 0.5 to 50 mM, especially 0.1 to 10 mM, Na₂HPO₄, 0.1 to 100 mM, preferably 0.5 to 50 mM, especially 0.1 to 10 mM, KH₂PO₄, 1 to 1000 mM, preferably 5 to 500 mM, especially 50 to 300 mM, NaCl, 0.1 to 100 mM, preferably 0.5 to 50 mM, especially 0.1 to 10 mM, KCl, and/or 0.01 to 1%, preferably 0.05 to 0.5%, especially 0.1 to 0.5%, aluminium adjuvant (aluminium hydroxide).
   68. Pharmaceutical composition according to any one of claims 1 to 67 having in a liquid formulation or in a dry or frozen formulation which is reconstitutable to a liquid formulation a pH of 4.5 to 7.4, preferably of 4.7 to 7.3, even more preferred of 5.0 to 7.3, especially of 5.3 to 7.2.
   69. Pharmaceutical composition comprising a P. acnes polypeptide or vaccine according to any one of embodiments 1 to 68, eliciting a cross-type reactivity at least against Types IA1, IA2, and II of P. acnes and/or eliciting a cross-type reactivity at least against Types IA1, IA2, IB, II and III of P. acnes.
   70. Pharmaceutical composition comprising a P. acnes polypeptide or vaccine according to any one of embodiments 1 to 69, wherein the polypeptide is present in the composition at least partially in a form wherein it lacks an N-terminal methionine residue.
   71. A vaccine comprising the putative iron-transport protein of P. acnes (PITP polypeptide) and/or a fragment and/or or a derivative of PITP, wherein PITP comprises from N- to C-terminus an extended neocarzinostatin family domain (“ENFD”), a first swapping region (“SR1”), a heme-binding domain (“HbD”), a second swapping region (“SR2”) including the C-terminal LPXTG motif, and a hydrophobic C-terminal region (“hLAR”), wherein the fragment and/or the derivative comprises or consists at least of a PITP epitope.
   72. Vaccine according to embodiment 71, wherein the fragment and/or derivative is a PITP polypeptide wherein the hLAR is deleted, replaced by a hydrophilic C-terminal region, or partially deleted, wherein the partial deletion results in a loss of hLAR except the N-terminal 12 amino acids of hLAR, preferably except the N-terminal 11 amino acids of hLAR, especially except the N-terminal 10 amino acids of hLAR; or a fragment thereof or derivative thereof comprising at least amino acids corresponding to proline 34 to glutamic acid 73 or proline 94 to threonine 143 of ENFD or valine 238 to asparagine 393 of HbD in the amino acid sequence Q6A9N1 in the UniProt database.
   73. Vaccine according to embodiment 71 or 72, wherein the fragment or the derivative comprises or consists at least of
a contiguous polypeptide sequence of proline 34 to glutamic acid 73 of ENFD,
a contiguous polypeptide sequence of proline 94 to threonine 143 of ENFD, or
a contiguous polypeptide sequence of valine 238 to asparagine 393 of HbD.
   74. Vaccine according to any one of embodiments 71 to 73, wherein the fragment or derivative contains a fragment of ENFD and/or of HbD of the PITP polypeptide with a length of at least 8 amino acids, preferably at least 9 amino acids, more preferred at least 10 amino acids, more preferred at least 15 amino acids, even more preferred at least 35 amino acids, even more preferred a length of at least 40 amino acids, especially a length of at least 50 amino acids.
   75. Vaccine according to any one of embodiments 71 to 74, which comprises at least one additional polypeptide with an amino acid sequence containing or consisting of at least one additional P. acnes antigen or P. acnes epitope which is not a PITP antigen or epitope, preferably at least one additional amino acid sequence containing at least the Dermatan sulfate-binding adhesin 1 of P. acnes (DsA1 polypeptide) and/or Dermatan sulfate-binding adhesin 2 of P. acnes (DsA2 polypeptide), and/or a fragment and/or a derivative of DsA1 or DsA2, wherein DsA1 and DsA2 comprise from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Thr repeat containing region (“PT repeat region”), and a C-terminal region (“CTR”), wherein the fragment and/or the derivative comprises or consists at least of a CSD2 fragment, wherein the CSD2 fragment is preferably
a contiguous polypeptide sequence of phenylalanine 150 (F150) to leucine 184 (L184),
a contiguous polypeptide sequence of phenylalanine 150 (F150) to leucine 267 (L267), or
a contiguous polypeptide sequence of histidine 218 (H218) to leucine 267 (L267),
   with the proviso that the fragment or derivative does not comprise an NSR and a CTR, and wherein the amino acid numbering corresponds to the amino acid sequence Q6A5X9 in the UniProt database.
   76. Vaccine according to embodiment 75 wherein the derivative comprises at least one additional sequence from the DsA1 or DsA2 polypeptide, preferably a sequence comprising at least one of CSD1, CSD2, CSD3 of DsA1 or DsA2 or an epitope-containing fragment thereof.
   77. Isolated fragment or derivative of the PITP polypeptide as defined in any one of embodiments 71 to 76, wherein the fragment and/or derivative is a PITP polypeptide wherein the hLAR is deleted, replaced by a hydrophilic C-terminal region, or partially deleted, wherein the partial deletion results in a loss of hLAR except the N-terminal 12 amino acids of hLAR, preferably except the N-terminal 11 amino acids of hLAR, especially except the N-terminal 10 amino acids of hLAR; or a fragment thereof or derivative thereof comprising at least amino acids corresponding to proline 34 to glutamic acid 73 or proline 94 to threonine 143 of ENFD or valine 238 to asparagine 393 of HbD in the amino acid sequence Q6A9N1 in the UniProt database.
   78. Fragment or derivative of the PITP polypeptide according to embodiment 77, wherein the fragment or the derivative comprises or consists at least of
a contiguous polypeptide sequence of proline 34 to glutamic acid 73 of ENFD,
a contiguous polypeptide sequence of proline 94 to threonine 143 of ENFD, or
a contiguous polypeptide sequence of valine 238 to asparagine 393 of HbD.
   79. Fragment or derivative of the PITP polypeptide according to embodiment 77 or 78, wherein the fragment or the derivative comprises or consists at least of an epitope of ENFD and/or an epitope of HbD, preferably wherein the fragment or the derivative comprises or consists at least of an epitope of ENFD and an epitope of HbD.
   80. Fragment or derivative of the PITP polypeptide according to any one of embodiments 77 to 79, wherein the derivative further comprises at least a ENFD and/or HbD from at least one other PITP from at least one other P. acnes strain than the strain from which the PITP fragment or derivative has been derived.
   81. Fragment or derivative of the PITP polypeptide according to any one of embodiments 77 to 80, wherein the fragment or derivative lacks the SR, corresponding to serine 180 (S180) to glutamine 198 (Q198), preferably at least the amino acid sequence corresponding to proline 179 (P179) to threonine 207 (T207), especially the amino acid sequence corresponding to glutamic acid 165 (E165) to lysine 237 (K237), or valine 401 (V401) to threonine 430 (T430), preferably serine 397 (S397) to T430, especially leucine 394 to T430; in the amino acid sequence Q6A9N1 in the UniProt database.
   82. Fragment or derivative of the PITP polypeptide according to any one of embodiments 77 to 81, wherein at least the amino acid sequence corresponding to leucine 427 (L427) to glycine 431 (G431) is deleted, preferably wherein at least the amino acid sequence corresponding to proline 179 (P179) to glycine 431 (G431) is deleted, especially wherein at least the amino acid sequence corresponding to threonine 392 (T392) to glycine 431 (G431), is deleted; according to the numbering in the amino acid sequence Q6A9N1 in the UniProt database.
   83. Fragment or derivative of the PITP polypeptide according to any one of embodiments 77 to 82, wherein at least the amino acid sequence corresponding to serine 180 (S180) to glutamine 198 (Q198) and/or phenylalanine 74 (F74) to serine 93 (S93), preferably at least the amino acid sequence corresponding to proline 179 (P179) to threonine 207 (T207), especially the amino acid sequence corresponding to threonine 159 (T159) to threonine 219 (T219), in the amino acid sequence Q6A9N1 in the UniProt database is deleted.
   84. PITP, and/or a fragment and/or a derivative of PITP according to any one of embodiments 71 to 83 for use in a therapeutic treatment, preferably for use in the treatment or prevention of P. acnes-associated infections, especially selected from the group consisting of acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes.
   85. PITP, and/or a fragment and/or a derivative of PITP according to any one of embodiments 71 to 83 for use in the treatment or prevention of P. acnes-associated infections in a human patient suffering from P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes.
   86. Pharmaceutical formulation, especially vaccine, according to any one of embodiments 1 to 85, comprising a PITP polypeptide and/or a porphyrin-binding PITP fragment and/or a porphyrin-binding PITP derivative, further comprising a porphyin molecule and/or a porphyrinogen molecule and/or a porphyrinogen or porphyrin degradation product bound to the PITP and/or a porphyrin-binding PITP fragment and/or a porphyrin-binding PITP derivative, wherein the porphyin molecule and/or porphyrinogen molecule and/or porphyrinogen or porphyrin degradation product preferably contains a metal ion in complex form, wherein the metal ion is preferably a Fe, Co, Zn, Mg, Se. or Cu ion, especially a Fe²⁺ or a Fe³⁺ ion.
   87. Pharmaceutical formulation, especially vaccine, according to any one of embodiments 1 to 86, comprising a PITP polypeptide and/or a porphyrin-binding PITP fragment and/or a porphyrin-binding PITP derivative, further comprising a porphyin molecule and/or a porphyrinogen molecule and/or a porphyrinogen or porphyrin degradation product bound to the PITP and/or a porphyrin-binding PITP fragment and/or a porphyrin-binding PITP derivative, wherein the porphyin molecule and/or porphyrinogen molecule and/or porphyrinogen or porphyrin degradation product is selected from the group hemin, protoprophyrin IX, coproporphyrin I, II and/or II, especially coproporphyrin II; pentacarboxyporphyrin, uroporphyrin, heptacarboxyporphyrin, hexacarboxyporphyrin, especially as form I or III; nitro-porphyrins, especially 5,10,15,20-tetraphenylporphyrin (TPP), 5,10,15,20-tetra(4′-fluorophenyl)porphyrin (TpFPP), 5,10,15,20-tetra(4′-chlorophenyl)porphyrin (TpClPP), 5,10,15,20-tetra(4′-bromophenyl)porphyrin (TpBrPP), biliverdin, 5,10-diphenyl-15,20-di(N-methylpyridinium-4-yl)porphyrin (Di-cis-Py+), PORF-1 to PORF-34, porphine, octaethylporphyrin, tetraphenylporphyrin, verteportin, ascoproporphyrin, hydroxymethylbilane (HMB), mesoporphyrin IX, 7-carboxyporphyrin (7P), 6-carboxylporphyrin (6P), heme A, heme B, heme C, heme O, heme I, heme m, heme D. S heme; uroporphyrinogen III, chlorophylls, especially chlorophyll a, chlorophyll b, and bacteriochlorophylls.
   88. Vaccine or pharmaceutical formulation as defined in any one of embodiments 1 to 87 for use in the treatment or prevention of P. acnes-associated infections, comprising a polypeptide comprising an epitope of DsA1 and/or DsA2 and/or PITP, wherein the epitope is selected from the group consisting of R32-141, Q38-K51. R32-K51, T43-K51. Q38-K51, R87-K90+T43-K51, R87-K90+T117-I132 and R87-K90+S234-G250, R87-K90+L246-A260, R87-K90+A256-E270, R87-K90+R266-T277, T117-I132, T117-A127, V128-I132, A144-N157, H146-A160, A148-N157, A156-A170, K166-L180, A176-T190, P186-A198, N181-E191, I216-F224, I216-D225, A226-A24G, S234-G250, I251-I263, I264-P271, P236-G250, L246-A260, A256-E270, S234-G250, I251-I263, I251-L267, A268-L280, R266-T277, T285-R286+I216-F224, T285-R286+I264-P271, T285-R286+V289-K296, T285-R286+V289-K296, T285-R286+A144-N157, A310-D313+T285-R286, T285-D290, T285-D290+V291-T300, T285-D290+A301-E307. V291-T300, A301-E307, T285-T300, A301-E307, R286-D290+V291-T300, R286-D290+A301-E307, R286-T300, V289-K296, A310-D313+I216-F224, A310-D313+I264-P271, A310-D313+T285-R286, A310-D313+R286-D290, A310-D313+V289-K296, A310-D313+V289-K296, A310-D313+T285-T300, A310-D313+A144-N157, A310-D313+T285-R286, A310-D313+T285-D290, A310-D313+T293-E307, T285-D290, V291-T300, T293-E307, A301-E307 of DsA1; L152-Q166, G190-P230, I199-D208, A218-I237, P230-Q244, I231-A270, H254-A270, A271-5279, A271-R310, L311-T321, L311-K323, V333-Q347, A218-P230, I231-I237, H254-H262, Q256-H262. E261-D269, D269-S279, K313-K323, of DsA2; D79-T90, E73-D85, R43-I50, P68-Y75, P86-E92, I39-G45, Y84-D89, F81-D89, D79-T90, T37-E44, E73-W98, E73-F81, D89-T90, P72-F8I, A129-F138, D120-Q134, F111-D120, F132-G147, D152-E165, R115-F123, D120-K128, P131-F138, N181-E191, T143-T159, P116-T124, P131-D137, P131-D137, T175-C231, Q198-K203, P179-K185, G200-Q210, K174-A188, K174-K185, P201-Q209, P183-P201, P183-K191, K185-P195, R164-S180, E165-S180, K185-S190, V193-N202, V193-G200, K203-P208, R216-T225, R216-R224, P173-K191, K197-K203, P168-T175, K185-K203, R164-K174, T175-V193, S250-N261, D287-S300, K340-V347, D338-F352. D338-D348, S285-P288+G305-L314, S285-P288+H306-L314, S285-P288+T342-T351, S285-P288+D338-D348, D287-S300, T342-T351, D338-D348, H306-L314, G305-L314, G364-K375, R382-E399, V367-G373, A383-L390, T342-T351, M387-T395, E385-T392, V401-V410, N404-A409, G416-L427, L396-V410, T406-I415, D417-G424, V407-D418, V407-V414, K421-V429, S419-T430, D408-1415, T406-V414, of PITP, preferably wherein the epitope comprises at least one additional amino acid residue at the N- or C-terminus of the DsA1, DsA2, or PITP sequence, especially wherein the epitope comprises at least two additional amino acid residue at the N- or C-terminus of the DsA1, DsA2, or PITP sequence; wherein the polypeptide is preferably covalenly linked to a carrier molecule or embedded in a scaffold molecule, especially a carrier polypeptide.
   89. Vaccine or pharmaceutical formulation as defined in any one of embodiments 1 to 87 for use in the treatment or prevention of P. acnes-associated infections, comprising a polypeptide comprising an epitope of DsA1 and/or DsA2 and/or PITP, wherein the polypeptide is a DsA1/DsA2/PITP derivative with the motif K[NYI] starting at position 5 or 6 of the polypeptide and/or having on DNA level the sequence AADUAU or AAVAUU at this coding position.
   90. Vaccine or polypeptide according to any one of embodiments 1 to 89, wherein the DsA1 and/or DsA2 and/or PITP, and/or a fragment or derivative of DsA1 or DsA2 or PITP is provided as DNA or RNA encoding an antigen or an epitope containing fragment or derivative thereof, preferably as mRNA vaccine, especially an mRNA vaccine formulated with cationic polymers, with an mRNA molecule with the following structure: 5′UTR-signal peptide-encoded antigen or epitope-3′UTR; or as a vector-based vaccine, especially with an adenovirus vector, an adeno-associated virus vector, a vesicular stomatitis virus vector, a Newcastle Disease virus vector, an alphavirus vector, a baculovirus vector, or a retrovirus vector, and/or wherein the coding sequence on DNA level has the initial coding DNA sequence ATGGTG; especially wherein the mRNA comprises a sequence as transcribed from SEQ ID NOs: 67 to 76.
   91. Pharmaceutical formulation comprising a vaccine or polypeptide according to any one of embodiments 1 to 90 comprising the polypeptide as defined in any one of these claims, i.e. a DsA1 and/or DsA2 and/or PITP, and/or a fragment and/or a derivative of DsA1 and/or DsA2 and/or PITP according to any one of embodiments 1 to 90 or mixtures thereof; and a pharmaceutically acceptable excipient or carrier.
   92. Method of treatment or prevention of P. acnes-associated infections in a human patient comprising administration of an effective amount of a DsA1 and/or DsA2 and/or PITP, and/or a fragment and/or a derivative of DsA1 and/or DsA2 and/or PITP according to any one of embodiments 1 to 91 or mixtures thereof in an effective amount to a patient in need thereof.
   93. Method of treatment or prevention of P. acnes-associated infections in a human patient suffering from P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes comprising administration of an effective amount of a DsA1 and/or DsA2 and/or PITP, and/or a fragment and/or a derivative of DsA1 and/or DsA2 and/or PITP according to any one of embodiments 1 to 91 or mixtures thereof in an effective amount to a patient in need thereof.
   94. Method according to claim 93, wherein administration is performed by intradermal, subcutaneous (s.c.), parenteral, intramuscular (i.m.), mucosal, transcutaneous or topical administration, preferably by intradermal, or intramuscular administration, especially by a syringe or by microneedeling devices.
   95. Method of production of the polypeptides, vaccines and formulations according to any one of embodiments 1 to 91, wherein the polypeptides comprising at least one P. acnes epitope as defined in these claims are expressed in a host cell, extracted and purified from these host cells, and, optionally, formulated and finished to a pharmaceutical formulation, especially a vaccine for use in the treatment or prevention of P. acnes-associated infections in a human patient.
   96. Vaccine according to any one of embodiments 1 to 91 comprising a polypeptide
   (a) comprising at least an epitope-containing fragment or derivative of CSD1 of DsA1 and at least an epitope-containing fragment or derivative of CSD2 of DsA2; and/or
   (b) comprising at least a polypeptide stretch comprising a CSD1 fragment or derivative of CSD1 of DsA1 of at least 30 amino acid residues, preferably at least 40 amino acid residues, and at least a polypeptide stretch comprising a CSD2 fragment or derivative of CSD2 of DsA1 of at least 30 amino acid residues, preferably at least 40 amino acid residues;
   (c) comprising at least
a contiguous polypeptide sequence of phenylalanine 150 (F150) to leucine 184 (L184),
a contiguous polypeptide sequence of phenylalanine 150 (F150) to leucine 267 (L267), or
a contiguous polypeptide sequence of histidine 218 (H218) to leucine 267 (L267) of DsA1, and
a contiguous polypeptide sequence of phenylalanine 194 (F194) to leucine 228 (L228),
a contiguous polypeptide sequence of phenylalanine 194 (F194) to leucine 311 (L311), or
a contiguous polypeptide sequence of histidine 262 (H262) to leucine 311 (L311) of DsA2,
   especially wherein the polypeptide comprises an amino acid exchange at one or more of C53, C319 and C321 of DsA1, and C97 and C363 of DsA2, if present in the polypeptide, preferably one or more of amino acid exchanges C53S, C319S and C321P of DsA1 and C97S and C363S of DsA2 and/or wherein in the fragment or derivative of DsA1 or DsA2 at least 5 PT repeats, preferably at least 10 PT repeats, especially at least 15 PT repeats, are deleted compared to a naturally occurring wild type DsA1/DsA2 polypeptide (“native DsA1/DsA2”), and wherein preferably at least one, more preferred at least two, more preferred at least three, even more preferred at least four, especially five, PT repeat(s) is/are present.
   97. Vaccine according to any one of claims 1 to 96, wherein the polypeptide further comprises a PITP polypeptide or a fragment or derivative of PITP comprising a polypeptide stretch of at least 30 amino acid residues comprising at least an epitope of PITP, preferably wherein the PITP polypeptide or a fragment or derivative of PITP comprises at least
a contiguous polypeptide sequence of proline 34 to glutamic acid 73 of ENFD or a PITP derivative thereof,
a contiguous polypeptide sequence of proline 94 to threonine 143 of ENFD or a PITP derivative thereof, or
a contiguous polypeptide sequence of valine 238 to asparagine 393 of HbD or a PITP derivative thereof;
   especially wherein the PITP derivative comprises an amino acid exchange exchange at positions C231 and C402.
   98. Method of production of the polypeptides, vaccines and formulations according to any one of embodiments 1 to 91, wherein the polypeptides comprising at least one P. acnes epitope as defined in these claims are expressed or produced as an mRNA in a host cell, extracted and purified from these host cells, and, optionally, formulated and finished to a pharmaceutical formulation, especially a vaccine for use in the treatment or prevention of P. acnes-associated infections in a human patient.
   99. Method according to embodiment 98, wherein the host cell is an eukaryotic or prokaryotic host cell, preferably a prokaryotic host cell, especially E. coli or L. lactis; or a host cell which is able to glycosylate the expressed polypeptides as defined in any one of embodiments 1 to 96.
   100. Method according to embodiment 98 or 99, wherein the host cell is naturally unable to synthesize porphyrins and wherein preferably porphyrins are added to the culture, to the lysis buffer, and/or to the purified protein.
   101. Method according to any one of embodiments 98 to 100, wherein the polypeptides of SEQ ID NOs:1 to 66 or encoded by SEQ ID NOs:67 to 76 are expressed in the host cells.

Therefore, the present invention also discloses the following embodiments, specifically drawn i.a to the aspect of the present invention wherein in a product, preferably a pharmaceutical product, especially a vaccine, at least two antigenic polypeptides with epitopes of P. acnes which are surface exposed are combined, and wherein preferably at least one epitope is an epitope of a dermatan sulfate-binding adhesin 1 of P. acnes (DsA1 polypeptide) which is surface exposed or an epitope of a dermatan sulfate-binding adhesin 2 of P. acnes (DsA2 polypeptide) which is surface exposed or an epitope of a putative iron-transport protein of P. acnes (PITP polypeptide) which is surface exposed:

1. Vaccine comprising at least two antigenic polypeptides with epitopes of P. acnes which are surface exposed, wherein preferably at least one epitope is an epitope of a dermatan sulfate-binding adhesin 1 of P. acnes (DsA1 polypeptide) which is surface exposed or an epitope of a dermatan sulfate-binding adhesin 2 of P. acnes (DsA1 polypeptide) which is surface exposed or an epitope of a putative iron-transport protein of P. acnes (PITP polypeptide) which is surface exposed,
wherein DsA1 comprises from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Thr repeat containing region (“PT repeat region”), and a C-terminal region (“CTR”), and wherein the PITP polypeptide comprises from N- to C-terminus an extended neocarzinostatin family domain (“ENFD”), a first swapping region (“SR1”), a heme-binding domain (“HbD”), a second swapping region (“SR2”) including the C-terminal LPXT(G) motif, and a hydrophobic C-terminal region (“hLAR”).
2. Vaccine comprising
at least one antigenic polypeptide with an epitope of a DsA1 polypeptide which is surface exposed or an epitope of a dermatan sulfate-binding adhesin 2 of P. acnes (DsA2 polypeptide) which is surface exposed,
   wherein DsA2 comprises from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), and, optionally, a Pro-Thr repeat containing region (“PT repeat region”), and a C-terminal region (“CTR”); and
at least one antigenic polypeptide with an epitope of a PITP polypeptide which is surface exposed.
   3. Vaccine comprising
at least one synthetic antigenic polypeptide with an epitope of a DsA1 polypeptide which is surface exposed or an epitope of a DsA2 polypeptide which is surface exposed; and
at least one synthetic antigenic polypeptide with an epitope of a PITP polypeptide of P. acnes which is surface exposed.
   4. Vaccine according to any one of embodiments 1 to 3 comprising the DsA1 polypeptide and/or the DsA2 polypeptide, and/or a fragment and/or a derivative of DsA1 or DsA2, wherein the fragment and/or the derivative of DsA1 or DsA2 comprises or consists at least of a CSD2 fragment, wherein the CSD2 fragment is preferably
a contiguous polypeptide sequence of phenylalanine 150 (F150) to leucine 184 (L184),
a contiguous polypeptide sequence of phenylalanine 150 (F150) to leucine 267 (L267), or
a contiguous polypeptide sequence of histidine 218 (H218) to leucine 267 (L267),
   with the proviso that the fragment or derivative does not comprise an NSR and a CTR, and wherein the amino acid numbering corresponds to the amino acid sequence Q6A5X9 in the UniProt database.
   5. Vaccine according to embodiment 4, wherein the fragment or the derivative of DsA1 or DsA2 comprises or consists at least of
a contiguous polypeptide sequence of phenylalanine 150 (F150) to isoleucine 193 (I193).
a contiguous polypeptide sequence of phenylalanine 150 (F150) to leucine 267 (L267), or
a contiguous polypeptide sequence of histidine 218 (H218) to leucine 267 (L267).
   6. Vaccine according to any one of embodiments 4 or 5, wherein the DsA1 or DsA2 fragment is
(1) a contiguous polypeptide sequence of phenylalanine 150 (F150) to leucine 184 (L184), further extending one, two, three, or four amino acids at the N-terminus and/or one, two, three, four, five, six, seven, eight, nine, or ten amino acids at the C-terminus;
(2) a contiguous polypeptide sequence of phenylalanine 150 (F150) to leucine 267 (L267), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus;
(3) a contiguous polypeptide sequence of histidine 218 (H218) to leucine 267 (L267) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus:
(4) a contiguous polypeptide sequence of phenylalanine 150 (F150) to leucine 267 (L267), further extending one, two, three, or four amino acids at the N-terminus and/or one, two, three, four, five, six, seven, eight, nine, or ten amino acids at the C-terminus;
(5) a contiguous polypeptide sequence of alanine 148 (F148) to leucine 271 (L271), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus;
(6) a contiguous polypeptide sequence of histidine 146 (H146) to threonine 277 (T277) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus;
(7) a contiguous polypeptide sequence of leucine 238 (L238) to leucine 272 (L272), further extending one, two, three, or four amino acids at the N-terminus and/or one, two, three, four, five, six, seven, eight, nine, or ten amino acids at the C-terminus;
(8) a contiguous polypeptide sequence of leucine 145 (L145) to alanine 201 (A201), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus; and/or
(9) a contiguous polypeptide sequence of leucine 145 (L145) to leucine 280 (L280) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus.
   7. Vaccine according to any one of embodiments 1 to 6, wherein the at least two antigenic polypeptides have a length of at least 35 amino acids, preferably a length of at least 40 amino acids, especially a length of at least 50 amino acids.
   8. Vaccine according to any one of embodiments 1 to 7 comprising a fragment or derivative of the PITP polypeptide, wherein the fragment and/or derivative of the PITP polypeptide is a PITP polypeptide wherein the hLAR is deleted, replaced by a hydrophilic C-terminal region, or partially deleted, wherein the partial deletion results in a loss of hLAR except the N-terminal 12 amino acids of hLAR, preferably except the N-terminal 11 amino acids of hLAR, especially except the N-terminal 10 amino acids of hLAR: or a fragment thereof or derivative thereof comprising at least amino acids corresponding to proline 34 to glutamic acid 73 or proline 94 to threonine 143 of ENFD or valine 238 to asparagine 393 of HbD in the amino acid sequence Q6A9N1 in the UniProt database.
   9. Vaccine according to embodiment 8, wherein the fragment or the derivative of the PITP polypeptide comprises or consists at least of
a porphyrin-binding domain of ENFD or HbD or a prophyin-binding derivative thereof,
a contiguous polypeptide sequence of proline 34 to glutamic acid 73 of ENFD,
a contiguous polypeptide sequence of proline 94 to threonine 143 of ENFD, or
a contiguous polypeptide sequence of valine 238 to asparagine 393 of HbD.
   10. Vaccine according to any one of embodiments 4 to 9, wherein the vaccine further comprises a fragment or derivative of DsA1 or DsA2 comprising or consisting of at least an NSR, CSD1, SR1, CSD2, SR2, CSD3 and/or CTR from at least one other DsA1 and/or DsA2 or from at least one other P. acnes strain than the strain from which the CSD2 fragment has been derived.
   11. Vaccine according to any one of embodiments 8 to 10, wherein the vaccine further comprises a fragment or derivative of the PITP polypeptide comprising or consisting of at least a ENFD or HbD from at least one other PITP polypeptide or from at least one other P. acnes strain than the strain from which the ENFD or HbD fragment has been derived, especially wherein the PITP polypeptide is a fragment or derivative consisting of the following amino acids: A32 to T430, A32 to G426, A32 to Q198, A32 to T143, A32 to K400, A32 to T159, A32 to I177, A32 to Q204, A32 to G234, A32 to R164, A32 to S391, A32 to P179, A32 to R158, A32 to G147, A32 to E73 and P94 to G147; P34 to T430, P34 to G426, P34 to Q198, P34 to T143, P34 to K400, P34 to T159, P34 to I177, P34 to Q204, P34 to G234, P34 to R164, P34 to S391, P34 to P179, P34 to R158, P34 to G147, P34 to E73 and P94 to G147; S240 to S391, A32 to D441, A32 to I440, A32 to E439, A32 to D438, A32 to S437. A32 to S436. A32 to P435, A32 to G434, A32 to E433, A32 to A432, A32 to G431; P34 to D441, P34 to 1440, P34 to E439, P34 to D438, P34 to S437, P34 to S436, P34 to P435, P34 to G434, P34 to E433, P34 to A432, P34 to G431; S240 to D441, S240 to I440, S240 to E439, S240 to D438, S240 to S437, S240 to S436, S240 to P435, S240 to 0434, S240 to E433, S240 to A432, S240 to G431; A32 to T430. A32 to V429, A32 to P428, A32 to L427, A32 to G426, A32 to R425, A32 to G424, A32 to A423, A32 to G422, A32 to K421, A32 to G420, A32 to S419, A32 to D418, A32 to D417. A32 to G416, A32 to I415. A32 to V414, A32 to K413. A32 to G412, A32 to T411, A32 to V410, A32 to A409, A32 to D408, A32 to V407, A32 to T406, A32 to V405, A32 to N404, A32 to H403, A32 to C402, A32 to V401, A32 to K400, A32 to E399, A32 to A398, A32 to S397, A32 to L396, A32 to T395, A32 to L394, A32 to N393, A32 to T392; P34 to T430, P34 to V429, P34 to P428, P34 to L427, P34 to G426, P34 to R425, P34 to G424, P34 to A423, P34 to G422, P34 to K421, P34 to G420, P34 to S419, P34 to D418, P34 to D417, P34 to 0416, P34 to 1415, P34 to V414, P34 to K413, P34 to G412, P34 to T411, P34 to V410, P34 to A409, P34 to D408, P34 to V407, P34 to T406, P34 to V405, P34 to N404, P34 to H403, P34 to C402, P34 to V401, P34 to K400, P34 to E399, P34 to A398, P34 to S397, P34 to L396, P34 to T395, P34 to L394, P34 to N393, P34 to T392; G172 to T430. G172 to V401, G172 to K400, 0172 to L396. G172 to N393, A199 to T430, A199 to V401, A199 to K400, A199 to L396, A199 to N393, H223 to T430, H223 to V401, H223 to K400, H223 to L396, H223 to N393, T232 to T430, T232 to V401, T232 to K400, T232 to L396, T232 to N393, G234 to T430, G234 to V401, G234 to K400, G234 to L396, G234 to N393, V238 to T430, V238 to V401, V238 to K400, V238 to L396, V238 to N393, S240 to T430, S240 to V429, S240 to P428, S240 to L427, S240 to G426, S240 to R425, S240 to G424, S240 to A423. S240 to 0422, S240 to K421, S240 to G420. S240 to S419, S240 to D418, S240 to D417, S240 to G416, S240 to I415, S240 to V414. S240 to K413, S240 to G412, S240 to T411, S240 to V41G, S240 to A409, S240 to D408, S240 to V407, S240 to T406, S240 to V405, S240 to N404, S240 to H403, S240 to C402, S240 to V401, S240 to K40G, S240 to E399, S240 to A398, S240 to S397, S240 to L396, S240 to T395. S240 to L394. S240 to N393, S240 to T392; 39I-48I, 40P-49T, 41V-50I, 42G-51S, 43R-52G, 44E-53K, 68P-77N, 69A-78S, 70S-79D, 71V-80K, 72P-81F, 73E-82Y, 74F-83G, 75Y-84Y, 76G-85D, 77N-86P, 78S-87S, 79D-88K, 80K-89D, 81F-90T, 82Y-91T, 83G-92E, 84Y-93S, 85D-94P, 86P-95S, 87S-96T, 88K-97I, 89D-98W, 90T-99V, 91T-100Y, 92E-101T, 93S-102P, 94P-103S, 95S-104Q, 96T-105K, 971-106A, 98W-107I, 99V-108G, 100Y-109S, 101T-110R, 102P-111F, 103S-112A, 104Q-113Q, 105K-114G, 106A-115R, 107I-116P, 108G-117M, 109S-118N, 110R-119N, 111F-120D, 112A-121G, 125I-134Q, I26T-135G, I27M-136K, I28K-137D, 144K-153H, 145A-154S, 146H-155D, 147G-156D, 148V-157T, 149G-158R, 150K-159T, 151T-160P, 152D-161V, 153H-162T, 154S-163Y, 155D-164R, 156D-165E, 157T-166A, 158R-167T, 159T-168P, 160P-169A, 161V-170P, 162T-171T, 163Y-172G, 164R-173P, 165E-174K, 166A-175T, 167T-176P, 168P-177I, 169A-178A, 170P-179P, 171T-180S, 172G-181K. 173P-182Q, 174K-183P, 175T-184S, 176P-185K, 177I-186Q, 178A-187A, 179P-188A, 180S-189P, 181K-190S, 182Q-191K, 183P-192Q, 184S-193V, 185K-194K, 186Q-195P, 187A-196S, 188A-197K, 189P-198Q, 190S-199A, 191K-200G, 192Q-201P, 193V-202N, 194K-203K, 195P-204Q, 196S-205S, 197K-206T, 198Q-207T, 199A-208P, 200G-209Q, 201P-210Q, 202N-211K, 203K-212T, 204Q-213A, 205S-214E, 206T-215H, 207T-216R, 208P-217S, 209Q-218Q, 210Q-219T, 211K-220P, 212T-221 A, 213A-222A, 214E-223H, 215H-224R, 216R-225T, 217S-226M, 218Q-227T, 219T-228K, 220P-229Q, 221A-230V, 222A-231C, 223H-232T, 224R-233I, 225T-234G, 226M-235A, 227T-236S, 228K-237K, 229Q-238V, 230V-239T, 231C-240S, 232T-241G, 233I-242S, 266L-275S, 267S-276A, 268G-277F, 282T-291K, 334S-343N, 353G-3621, 354V-363K, 355S-364G, 356V-365S, 357S-366P, 358G-367V, 359N-368K, 377F-386P, 378A-387M, 379G-388N, 380F-389P, 396L-405V, 397S-406T, 398A-407V, 399E-408D, 400K-409A, 401V-410V, 402C-411T, 403H-412G, 404N-413K, 405V-414V, 406T-4151, 407V-416G, 408D-417D, 409A-418D, 410V-419S, 411T-420G, 412G-421K, 413K-422G, 414V-423A, 415I-424G, 416G-425R, 417D-426G, 418D-427L, 419S-428P, 4200-429V, 421K-430T, 422G-431G. 423A-432A, 424G-433E, 425R-434G, 426G-435P, 427L-436S, 428P-437S, 429V-438D, 430T-439E, 431G-440I. 432A-441D, 433E-442L, 434G-443G, 435P-444I, 436S-445V, and 437S-446G; I39-K53, P68-G121, I125-D137, K144-S242, L266-F277, T282-K291, S334-N343, G353-K368, F377-P389, L396-G446, A32 to R164, A32 to Q198, A32 to T143, A32 to V148. A32 to T171, P34 to R164, A32 to T159, A32 to I177. A32 to Q204. A32 to G234, A32 to K400, A32 to S391, V238 to K400, A199 to T430, V238 to T395, G234 to K400, H223 to K400, T232 to V401, V238 to T392, V238 to N393, V238 to L394, V238 to T395. V238 to L396, T232 to T430, G172 to K400, and G172 to G234, and fragments thereof comprising at least of a PITP epitope.
   12. Vaccine according to any one of embodiments 1 to 11 further comprising another P. acnes antigen or epitope, preferably an antigen or epitope selected from DsA1. DsA2 and/or PITP and/or a DsA1 epitope, a DsA2 epitope and/or a PITP epitope.
   13. Vaccine according to any one of embodiments 1 to 12 further comprising an epitope containing fragment of another P. acnes polypeptide.
   14. Vaccine according to any one of embodiments 1 to 13, wherein the vaccine is protective against development of a pathological condition induced by P. acnes invasion.
   15. Vaccine according to any one of embodiments 1 to 14, wherein the vaccine is formulated for administration to a human subject, e.g. a child, an adolescent or adult subject.
   16. Vaccine according to any one of embodiments 1 to 15, wherein the vaccine is formulated for use in a treatment regimen involving both, a prime and boost immunization, preferably wherein the same formulation is used for the prime and boost administration.
   17. Vaccine according to any one of embodiments 1 to 16, wherein the vaccine is formulated with an adjuvant, preferably with an adjuvant selected from the group consisting of mineral salts, preferably alum (potassium aluminum sulfate), aluminum hydroxide, aluminum phosphate, aluminum oxohydroxide, aluminum hydroxyphosphate, calcium phosphate, cerium nitrate, zinc sulfate, colloidal iron hydroxide, and/or calcium chloride; oil-in-water emulsions, liposomes, TLR agonists, Monophosphoryl Lipid A, saponins, phospholipids, emulsion-based adjuvants, aluminium hydroxide gel, solid phase adsorbents, nanospheres and encapsulating materials such as liposomes; high molecular weight adjuvants, preferably toxins, toxoids or any mutant cross-reactive material of the toxin from tetanus, diphtheria, pertussis, Pseudomonas species, E. coli, Staphylococcus species, and Streptococcus species. Such toxins or toxoids may be tetanus toxoid, pertussis toxoid, cholera toxoid, E. coli LT, E. coli ST, and exotoxin A from Pseudomonas aeruginosa; bacterial outer membrane proteins such as outer membrane protein complex c (OMPC), porins, transferrin binding proteins, pneumolysin, pneumococcal surface protein A (PspA), pneumococcal adhesin protein (PsaA), C. difficile enterotoxin (toxin A) and cytotoxin (toxin B) or Haemophilus influenzae protein D, other pharmaceutically acceptable polypeptide carriers, such as ovalbumin, keyhole limpet hemocyanin (KLH), bovine senum albumin (BSA) or purified protein derivative of tuberculin (PPD); cationic peptides, CpG oligonucleotide, squalene-based adjuvants, preferably MF59; cytokines, such as IL-1 and IL-2; or formulated as virus-like particles or nanoparticles; or combinations thereof.
   18. Vaccine according to any one of embodiments 1 to 17, wherein the vaccine is formulated for intradermal, subcutaneous (s.c.), parenteral, preferably intramuscular (i.m.), mucosal, transcutaneous or topical administration.
   19. Vaccine according to any one of embodiments 1 to 18, wherein the vaccine comprises 0.1 μg to 5 mg, preferably 0.5 to 1000 μg, more preferred 1 to 500 μg, even more preferred 5 to 300 μg, especially 10 to 200 μg, each of the DsA1 and/or DsA2 and/or PITP, and/or a fragment or derivative of DsA1 or DsA2 or PITP per dose.
   20. Vaccine according to any one of embodiments 1 to 19, wherein the DsA1 and/or DsA2 and/or PITP, and/or a fragment or derivative of DsA1 or DsA2 or PITP is provided as DNA or RNA encoding an antigen or an epitope containing fragment or derivative thereof, preferably as mRNA vaccine, especially an mRNA vaccine formulated with cationic polymers, with an mRNA molecule with the following structure: 5′UTR-signal peptide-encoded antigen or epitope-3′UTR; or as a vector-based vaccine, especially with an adenovirus vector, an adeno-associated virus vector, a vesicular stomatitis virus vector, a Newcastle Disease virus vector, an alphavirus vector, a baculovirus vector, or a retrovirus vector; and/or wherein the coding sequence on DNA level has the initial coding DNA sequence ATGGTG; especially wherein the mRNA comprises a sequence as transcribed from SEQ ID NOs: 67 to 76.
   21. Vaccine according to any one of embodiments 1 to 20, wherein the vaccine comprises 5 to 500 μg each, preferably 20 to 100 μg each, of DsA1 and/or DsA2 and/or PITP, and/or a fragment or derivative of DsA1 or DsA2 or PITP; or DNA or RNA encoding an antigen, or a fragment or a derivative thereof, per dose.
   22. Vaccine according to any one of embodiments 1 to 20, wherein the vaccine comprises less than 1 μg each of DsA1 and/or DsA2 and/or PITP, and/or a fragment or derivative of DsA1 or DsA2 or PITP per dose.
   23. Vaccine according to any one of embodiments 1 to 22, wherein the vaccine does not contain Freund's incomplete adjuvant.
   24. Vaccine according to any one of embodiments 1 to 23, wherein the vaccine contains a composite immunogen and is engineered by linking of one or more antigen(s) of P. acnes and/or one or more cross-reactive, especially cross-type-reactive, or otherwise immunologically relevant epitope(s) of P. acnes to each other, preferably wherein the immunogen comprises or consists of at least 2 (e.g. 2, 3, 4, 5, 6, 7, 8, 9, or 10) antigen(s) and/or immunologically relevant epitope(s) thereof, especially wherein the antigens and/or epitopes are cross-reactive, especially cross-type-reactive.
   25. Vaccine according to any one of embodiments 1 to 24, wherein the vaccine contains a composite immunogen comprising or consisting of at least two (e.g., 2, 3, 4, 5, 6 7, 8, 9, or 10) antigen(s) of P. acnes and/or epitope(s) thereof linked to an adjuvant, wherein at least one antigen or epitope is immunologically relevant.
   26. Vaccine according to any one of embodiments 1 to 25, wherein the vaccine contains a composite immunogen comprising at least two antigen(s) and/or epitopes thereof of P. acnes covalently linked to each other, preferably wherein the immunogen comprises at least two antigen(s) and/or epitopes thereof further linked to an adjuvant, especially wherein at least one antigen or epitope is immunologically relevant.
   27. Vaccine according to any one of embodiments 1 to 26, wherein the vaccine contains a composite immunogen wherein one antigen or epitope thereof is linked to an adjuvant, especially wherein the at least one antigen or epitope is immunologically relevant.
   28. Vaccine according to any one of embodiments 1 to 27, wherein the vaccine comprises a variant of an antigen or of an epitope.
   29. Vaccine according to any one of embodiments 1 to 28, wherein the vaccine comprises a polypeptide with at least about 90% amino acid sequence identity, preferably at least 95%, more preferably at least 98%, more preferably at least 99% or at least 99.5%, to the amino acid sequence of a naturally occurring P. acnes antigen; or a polypeptide of a P. acnes epitope, wherein at least 1, 2, 3, 4, up to 5 point mutations are contained in the amino acid sequence compared to the naturally occurring epitope.
   30. Vaccine according to any one of embodiments 1 to 29, wherein the vaccine comprises a variant, preferably a DsA1 and/or DsA2 protein wherein one or more amino acid residues are added, or deleted, at the N- or C-terminus, as well as within one or more internal domains, especially a variant comprising additional amino acids at the N-terminal and/or at the C-terminal end, to prolong an antigen sequence. e.g. to prolong a sequence of an epitope or epitopic region within a protein by at least one amino acid residue, preferably by less than 3 amino acids, specifically less than 5, or else less than 10 amino acids.
   31. Vaccine according to any one of embodiments 1 to 30, wherein the vaccine comprises a derivative, especially as a fusion protein, wherein a PITP fragment containing a PITP epitope is prolonged by additional amino acid residues of another polypeptide or protein, preferably by one or more immunologically relevant epitopes, especially wherein the derivative comprises a His-tag at the N- or at the C-terminus comprising at least 4, preferably at least 5, especially at least 6 histidine residues.
   32. Vaccine according to any one of embodiments 1 to 31, wherein the vaccine comprises a fragment or derivative of DsA1 or DsA2, wherein DsA1 and DsA2 comprise from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Leu repeat containing region (“PT repeat region”), and a C-terminal region (“CTR”), wherein fragment or derivative comprises or consists at least of (1) a contiguous polypeptide sequence of phenylalanine 150 (F150) to leucine 184 (L184), (2) a contiguous polypeptide sequence of phenylalanine 150 (F150) to leucine 267 (L267), or (3) a contiguous polypeptide sequence of histidine 218 (H218) to leucine 267 (L267), with the proviso that the fragment or derivative does not comprise the NSR and the CTR, said fragment or derivative optionally comprising one or more amino acid exchanges, wherein one or more amino acid residues are changed to a cysteine (Cys, C) residue to further stabilise the fragment or derivative by the ability to form disulfide bonds.
   33. Vaccine according to any one of embodiments 1 to 32, wherein the fragment or derivative has a length of at least 20, preferably at least 30, more preferred at least 60 amino acids, even more preferred a length of at least 80 amino acids, especially a length of at least 100 amino acids.
   34. Vaccine according to any one of embodiments 1 to 33, wherein the fragment or derivative has a length of less than 400 amino acids, preferably less than 350 amino acids, especially less than 300 amino acid residues.
   35. Vaccine according to any one of embodiments 1 to 33, wherein the fragment or derivative has a length of less than 250 amino acids, preferably less than 200 amino acids, especially less than 150 amino acid residues.
   36. Vaccine according to any one of embodiments 1 to 35, wherein two or more amino acid residues in a fragment or derivative are changed to a cysteine (Cys, C) residue to further stabilise the fragment or derivative by the ability to form disulfide bonds, preferably (with respect to the DsA1 sequence (which is also applicable to the DsA2 sequence; FIG. 12A)) containing at least one of the amino acid pairs in the following group wherein the native amino acid is exchanged to cysteine: PHE150-GLY185, LEU167-ALA199, ASN157-ALA192, LYS166-ALA199, ALA153-GLY 185, ASP164-ALA199, VAL154-ALA179, PHE150-LEU184, VAL154-ALA188, ALA153-ALA188, ALA179-GLU191, THR178-GLU191, ALA161-ALA195, ILE175-ALA195, ILE175-GLU191, LEU158-ALA176, ASN157-ALA188, ASN157-ALA179, ASP164-GLU196, VAL154-LEU180, ALA153-LYS189, ASN157-LYS189. ALA161-ALA192, LEU167-ALA198, LEU158-ALA179, ALA160-ALA192, LYS165-ALA199, LYS166-GLN200; VAL205-ALA260, ILE 232-ARG266, SER235-ILE263, SER235-ARG262, ILE232-LEU267, PRO236-ILE263, ALA231-ARG266, ASN239-ILE259, ILE232-ILE263, VAL209-PRO236, ALA201-ALA256, SER235-ILE259, VAL228-ARG266, ALA202-ALA256, ALA201-VAL257, GLY206-VAL243, GLY208-ILE264, GLY208-ILE263, VAL205-ASN239, LEU238-ILE259, GLY208-ALA260, ALA201-LYS253, ALA202-VAL243, LEU204-ALA260, VAL205-ALA256, VAL205-ILE259, SER235-ARG266, ASN239-ALA260, VAL209-ILE263, or VAL209-ASN239.
   37. Vaccine according to any one of embodiments 1 to 36, wherein a cysteine in the DsA1, DsA2 or PITP or fragment or derivative thereof is exchanged, preferably wherein the cysteine is exchanged with serine, proline, alanine, threonine, asparagine, glutamine, valine, isoleucine, leucine, phenylalanine, tyrosine and glycine, more preferred with serine, proline, alanine, threonine, asparagine, glutamine, valine, isoleucine, leucine, especially with serine, proline, alanine, threonine, asparagine, and glutamine.
   38. Vaccine according to any one of embodiments 1 to 38, wherein the fragment or derivative has a length of length of 35 to 350 amino acids, preferably of 40 to 300 amino acids, more preferred of 50 to 250 amino acid residues, more preferred of 60 to 200 amino acids, more preferred of 70 to 150 amino acids, especially of 90 to 130 amino acid residues.
   39. Vaccine according to any one of embodiments 1 to 37, wherein the derivative comprises at least one polypeptide stretch of DsA1 and at least one polypeptide stretch of DsA2, preferably wherein the derivative comprises at least CSD1, CSD2 or CSD3 of DsA1 and at least CSD1, CSD2 or CSD3 of DsA2.
   40. Vaccine according to any one of embodiments 1 to 39, wherein the derivative comprises at least one fragment comprising at least CSD1, CSD2 or CSD3 of DsA1 and at least one fragment comprising at least CSD1, CSD2 or CSD3 of DsA2 and wherein the at least two fragments are interconnected by
an SR1 or SR2 of DsA1 or DsA2 adjacent to the at least CSD1, CSD2 or CSD3 of DsA1 and at least CSD1, CSD2 or CSD3 of DsA2; or
an amino acid sequence consisting of an N-terminal part of an SR1 or SR2 of DsA1 or DsA2, adjacent to the at least CSD1, CSD2 or CSD3 of DsA1 and at least CSD1, CSD2 or CSD3 of DsA2, and a C-terminal part of a different SR1 or SR2 of DsA1 or DsA2, adjacent to the at least CSD1, CSD2 or CSD3 of DsA1 and at least CSD1, CSD2 or CSD3 of DsA2, wherein the total number of amino acid residues the amino acid sequence consisting of the of the N- and C-terminal part is identical to the SR1 or SR2 of DsA1 or DsA2.
   41. Vaccine according to any one of embodiments 1 to 40, wherein the derivative comprises at least one fragment comprising at least CSD1, CSD2 or CSD3 of DsA1 and at least one fragment comprising at least CSD1, CSD2 or CSD3 of DsA2, wherein the fragment of DsA1 and DsA2 has independently a length of at least 20 amino acid residues, preferably at least 30, more preferred at least 40, especially at least 50, amino acid residues
   42. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP according to any one of embodiments 1 to 41 for use in a therapeutic and prophylactic treatment, preferably for use in the treatment or prevention of P. acnes-associated infections, especially selected from the group consisting of acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, artherosclerosis, prostethic cancer and a medical implant biofilm infection by P. acnes.
   43. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP according to any one of embodiments 1 to 41 for use in the treatment or prevention of P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes.
   44. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to embodiment 42 or 43, wherein the human patient is suffering from P. acnes-associated infections and pathological conditions associated with at least two P. acnes MLST phylotypes, and/or at least two different CC types of P. acnes and/or at least two ribotypes of P. acnes, as determined by a surface binding measurement employing flow cytometry and in a bactericidal killing assay using immune serum raised against such vaccine antigen.
   45. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 44, wherein the human patient is suffering from P. acnes-associated infections and pathological conditions associated with at least two or three genetically different types of P. acnes strains, preferably against at least one Type I and at least one Type II or III strains; or from at least one Type I and at least one Type I or III strains; or from at least one Type III and at least one Type I or II strains; or from at least one Type I, at least one Type II, and at least one Type III strain.
   46. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 45, wherein the human patient is suffering from P. acnes-associated infections and pathological conditions associated with two or more of P. acnes strains selected from the group consisting of Type IA1, Type IA2, Type IB, Type IC, Type II and Type III strains, or Type I-la, I-lb, I-2 and II, especially Types IA1, IA2, IB, II and III, as defined according to the MLST typing schemes.
   47. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 46, wherein the human patient is suffering from P. acnes-associated infections and pathological conditions associated with two or more of P. acnes strains selected from the group of the ribotypes as determined according to 16S ribosomal sequence differences.
   48. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 47, wherein the human patient is suffering from P. acnes-associated infections and pathological conditions associated with two or more phylotypes IA1, IA2, IB1, IB2, IB3, IC, II and III, especially Types IA1, IA2, IB, II and III, determined by the analysis of a single locus (SLST).
   49. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 48, wherein the human patient is suffering from P. acnes-associated infections and pathological conditions associated with at least two different phylogenetic types, more preferably three, even more preferably four, five or six phylogenetic types of the following group of strains: NCTC737, KPA171202 (DSMZ, German Collection of Microorganisms and Cell Cultures, Braunschweig, Germany), SK137, HL005PA1, HL005PA4, HL013PA1, HL030PA1, HL043PA1, HL053PA1, HL053PA2, HL050PA1, HL050PA2, HL060PA1, HL110PA4 (BEI, Biodefense and Emerging Infections Research Resources Repository, Manassas, VA), P.acn31 and Asn12 (McDowell et al. 2012) and IAI 008, IAI031, IAI034, IAI035, IAI038, IAI040, IAI042, IAI045, IAI041 (Charitt Berlin, Pro-Implant foundation).
   50. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 49 for use in protection against infection with P. acnes.
   51. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 50 for use in prevention or reducing development of a P. acnes-associated indication.
   52. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 51, wherein the vaccine repeatedly administered to a human patient, preferably by at least 2 or 3 administrations, or at least 4, 5, or even more repeated administrations.
   53. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 52, wherein the vaccine is used in one or more treatment cycles, each involving at least two, or three or four consecutive administrations within a period of 1 year in intervals of at least 1 or 2 weeks, especially wherein the treatment cycles are repeated at least any of 1×, 2×, 3×, or 4×, even more times. e.g. within a period of 5 years.
   54. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 53, wherein the vaccine provokes an immune response, including the production of antibodies and/or cytokines and/or the activation of cytotoxic T cells, B-cells, antigen presenting cells, helper T cells, dendritic cells and/or other cellular responses in an individual upon administration.
   55. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 54, wherein the vaccine is administered to a patient with a skin or other affected organ or tissue condition in which the strains from any of Type I, II, or III P. acnes, or a combination of at least two phylotypes of Type I, II, and III, or of at least two ribotypes of P. acnes, especially Types IA1, IA2, IB, II and III, are involved as pathogenic factors, or to a healthy individual that may be susceptible to an infection by any of the P. acnes strains.
   56. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 55, wherein the vaccine is administered to a patient by intradermal, transdermal or a subcutaneous administration, preferably by intradermal injection applicators, microneedles, transdermal laser devices, topical, skin patch or other suitable skin-adapted application device.
   57. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP for use according to any one of embodiments 42 to 55, wherein the vaccine is administered by oral or parenteral administration such as intramuscular, intranodal, intranasal, mucosal, intrapulmonary, vaginal, rectal or other suitable routes.
   58. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP as defined in any one of embodiments 1 to 57, preferably comprising at least one of the polypeptides according to SEQ ID NOs 4 to 21 and 20 to 50.
   59. Pharmaceutical composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP as defined in any one of embodiments 1 to 57, preferably comprising at least one of the polypeptides according to SEQ ID NOs 4 to 21 and 20 to 50.
   60. Pharmaceutical composition according to embodiment 59, further comprising a pharmaceutically acceptable carrier.
   61. A method for producing a vaccine, wherein at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP as defined in any one of embodiments 1 to 57 are mixed with a pharmaceutically acceptable excipient or carrier and, optionally, finished into a pharmaceutically administrable vaccine dose, preferably in a dosage unit suitable for administration to an individual, especially a human individual.
   62. Vaccine, method or composition according to any one of embodiments 1 to 61 comprising a polypeptide, comprising or consisting of
   (a) a CSD2 fragment of DsA1 or DsA2 within A148 to L267 of DsA1 or A192 to L311 of DsA2, at least comprising a contiguous polypeptide sequence of F150 to L184 of DsA1 or F194 to L228 of DsA2,
   (b) a CSD2 derivative of (a) with one or more, preferably one, two or three, especially one, of the following amino acid exchanges: A149T, D152N, D152S, V155I, V163I, K166H, K166P, A168T, K169R, T171A, V173M, A176V, A183T, T190F, T190I, E191K, A192L, A192F, A198G, A199T, A202T, V205I, G206N, G206S, G206R, G206Q, K212Q, A214T, I219V, I219A, S223A, D225N, V233I, S235F, N239S, L255I, V257I, Q258S, I259L, R262H, I264V, D265K in DsA1 and T193A, N196D, N196S, I199V, I207V, H210K, H210P, A212T, R213K, T215A, V217M, V220A, T227A, F234T, F234I, E235K, L236A, L236F, A242G, A243T, A246T, I249V, R250G, R250S, R250N, G250Q, Q256K, A258T, V2631, V263A, A267S, D269N, I277V, S279F, S283N, I299L, I301V, S302Q, L303I. H306R, V308I, K309D in DsA2
   (c) a CSD1 fragment of DsA1 or DsA2 within I49 to L130 of DsA1 or 193 to L174 of DsA2, at least comprising a contiguous polypeptide sequence of 20 amino acid residues, especially T117 to A127 of DsA1 or T161 to LA171 of DsA2,
   (d) a CSD1 derivative of (c) with one or more, preferably one, two or three, especially one, of the following amino acid exchanges: D50N, D50S, K51E, K51A, C53Y, D55K, V57I, A61L, A65G, A68V, L71M, F76L, S78A, V82M, V82A, L84M, P86R, P86S, K90R, K90A, K94A, V97T, L99T, I100L, K104R, K106E. A107V, I109V, G110A, A111S, L113V, G114S. L116V, T117A, K120A, I121V, R123H, A124T, V128I in DsA1 and S94D, S94N, E95A, E95K. C97Y, K99D, I101V, L105A, G109A, V112A, L115M, I20F, A122S, A126M, A126V, L128M, P130R, P130S, A134K, A134R, A138K, T141V, T143L, I144L, R148K, K150E, V151A, V153I, A154G, S155A, L157V, G158S, V160L, A161T, A164K, V165I, H167R, A168T, I172V in DsA2
   (e) a CSD3 fragment of DsA1 or DsA2 within A278 to K323 of DsA1 or A322 to E366 of DsA2, at least comprising a contiguous polypeptide sequence of R266 to T277 or R286 to K296 of DsA1 or V333 to Q347 of DsA2
   (f) a CSD3 derivative with one or more, preferably one, two or three, especially one, of the following amino acid exchanges:
   M281V, N283D, T285A, R286Q, V289A, V291I, I292R, T293N, A294T, D295K, D295Q, K296E, I298V, K299A, T300V, T300I, A301Y,—between 301 and 302 D, E302K, E305K, K306A, A310T, D3130, K316Q, S320T, C321—, P322L, K323D, K323E in DsA1 and V325M, D327N, A329T, R330Q, V333A, I335V, R336I, N337T, T338A, Q339K, Q339D, E340K, I342V, A343K, V344T, V344I, Y345A, K346E, K349E, A350K, T354A, G357D, Q360K, T364S, P365L, E366D, E366K, in DsA2.
   63. Vaccine, method or composition according to any one of embodiments 1 to 62 wherein comprising a DsA1 and/or DsA2 derivative, wherein the DsA1 and/or DsA2 derivative preferably comprises one or more, preferably one, two or three, especially one, of the following amino acid exchanges:
   DsA1: A301Y, G206L, G206F, A192L, G206R, G206W, G206Q, G206K, A301F, A301L, G206I, A301W, A301I, G206E, G206M, G208L, G206V, A192M. A256L, A1921. A68I, G206N, A68V, I259L, A161L, V205I, 0206T, G206H, A68L, G206Y, A256I, G206A, 68M, A192F, G110A, N239L, G206C, A301V, E305L, A93I, A93L, S223A, E305I, E305V, E305A, K120V, K120L, N157I, K120C, E305C, E305M, N239I, K120I, R248C, N239V, E191I, E305F, S78I, K120M, E191L, R248L, H146V, E191V, H146I, S78L, S78V, N157V, H146C, K120F, R248I, R248M, D164L, P236V, P236L, H146L, H146F, D164I, H146M, P86L, R248G, N239C, N157C, E305T, E191C, S78C, R248F, G221L, E305G, P236F, T171L, P86V, R248S, N157L, K120A, H146A. P86I, N157F, S235A, R248A, E191M, N157M, A124I, P236A, S78F, P236C, S78M, A161I, N239M, T171I, E191F, S235V, P86F, H146G, S223I, E305S, D164F, E305Y, P236M, S223L, N239F, H146Y, P236I, G221V, D164C, K120G, D164M, S235I, R248V, A124V, S235L, S223V, S147L, S235C, P86M, G208I, D164A, S147I, N239A, K120T, T117I, G110L, R248Y, P182I, A198L, A124L, A215L, A127L, A66L, P86C, G221F, R248T, T171F, P182V, S147V, P271V, T171V, G110V, P271A, E191A, A149L, K120Y, G114I, K120S, S235F, A201I, G131V, A201V, R248N, A66V, A202I, G221C, G114V, A229L, H146T, A124F, A127F, T117V. S223C, A215F, A127I, P182C, A215V, G162L, G162I, G208F, A201L, A161F, N157A, A198V, E191Y, A161V, S223F, G221M, R248W, G131C, A202V, N157T, S147F, N157Y, G114A, T171C, H146W, S78T, A149I, G250L, N239T, A149V, G208C, K120P, G250V, S235G, S223M, D164V, N239G, T171M, E191G, S78A, D164Y, P271I, G110F, G110C, D164W, P236Y, A124C, A66F, S147M, G75L, E305N, S147C, G221I, A127M, P182L, G221A, G110M, A66C, E305Q, P271F, A215M, P236W, K120W, H146P, A161C, A2291, S78G, G114M, P86A, E305W, V154L, G185V, G75I, S223Y, G75V, R248Q, T277V, E191S, A229F, T277G, G131I, N239S, P271C, V154I, A66M, D164G, G208A, P236T, R248D, D164T, A127C, A215I, A161M, P86W, A201F, C53L, T117F, G131A, T117C, A318L, F150L, G208M, P271M, A149C, G208V, G114C, G162F, S223W, A198I, N157G, A202L, P236G, 0185I, A198F, A149M, P86Y, E191W, N157W, I263L, S235T, E305P, E305R, A201C, A201M, F150I, G96I, G114F, G221Y, A199L, D164S, A124M, A179I, A202F, H146S, A231L, C531, 1263V, G162M, K120Q, N239Y, G96L, 075C, N157S, T277C, T1117A, A199V, G162C, P271Y, G250C, A127Y, A66I, T117L, G185C, A229C, R248P, A144I, T117M, P86T, A260L, G185A, E191Q, G131L, T277I, G250I, G131M, T277A, A127V, F150V, A318I, S78W, S147Y, A198M, P86G, A215C, A70I, S235M, A179V, A202M, A144V, A202C, G208W, T171Y, S147W, G208Y, G250F, A195L, K120H, A127W, A66Y, S223G, K120N, S78Y, E191N, A198C, P271G, V154F, A70V, H146N, A229M, A134L, P86S, S78Q, A199I, G75M, A68C, E191T, A89L, 096A, D164P, C53V, S78P, G185L, A149G, A149F, P182A; DsA2: A161L, A161I, A126I, A126V, A126L, S175G. G190H, A161T, S183T, R336I, A126F, A161F, A329L, A161V, S283L, A205L, G109A, A329I, G265F, A161W, A2321, S175W, R336L, A300I, K349I, A161M, A300L, G109L, G109I, A246L, S183M, A223L, E235V, G252L, A329M, A126W, S183L, A259L, A329V, A329F, S183I, A242L, G109F, R336V, K117I, R336C, K117C, K349V, R336A, R336F, K117L, R336M, E235I, R310A, K349L, K117V, R310V, K349C, N201I, R310I, R310C, K349F, K349M, K117M, R292C, N201V, R336G, R292L, K117F, E235C, P130L, E235A, D208L, P280L, E235L, R292M, R336T, P280V, R2921, N201L, E235M, P130I, K117A, K349A, P130V, R292G, R310F, R336S, R336Y, N201C, K117G, R310L, R310M, R310G, R336W, D208V, R292F, P130F, K349Y, K117T, R310T, P280C, R292S, N201F, R310S, T215L, N201M, S279C, R292A, S279L, P280F, D208C, S175V, A126M, P280A, S279A, P130C, S279V, E235F, P186C, P130M, K349T, A126C, K349G, N320V, K117S, N320I, S283V, A168I, N320L, S175I, E235Y, P186V, P280I, P186A, P280M, G265L, D208M, P186L, K117Y, S283I, A205I, D208F, A168L, T215I, R292V. A133L, R336P, R310Y, A168V, S279F, S183V, A164I, S279I, S175L, D208A, K349S, E235T, S175C, A122I, D208I, A126Y, A123L, K117P, N320C, R292T, S283C, E235W, G252I, T215V, R292Y, R336Q, S175A, A123I, E235G, A205V, A171V, A133V, A245I, S183C, P226V, R310P, G1811, P226I, A168C, A246I, A171L, N201A, S175M, G158I, A164L, T215C, A164C, P280W, A133C, G119I, T215F, A245V, N320M, A122V, A123V, G206L, N320A, G265V, A122L, A193L, S191V, R310W, G252F, G181V, E235S, R292N, P130W, G119V, G265C, S279M, A259V, A259F, A168F, A168M, A246V, P226C, R292W, G158V, P130Y, G206V, P186G, C97L, N201Y, A171C, G265M, G140L, K117N, G140I, D208G, A193V, G181F, K349W, S279G, A245L, A205F, G158A, A193I, K117W, A122C, R310D, G294V, P186T, S175F, S191C, N201T, K349Q, R310N, A242V, T215M, D208Y, T321C, G252C, 1276L, A259M, A171F, N201G, G119F, D208T, P186M, G229V, G294L, S191I, P186S, G119C, G2291, P130A, A205C, A171M, T321V, A193C, F268L, A133M, A123F, G357A, G109V, K117Q, N320G, R310Q, T321G, S283M, P186Y, G229A, R336D, N201S, S183F, F194L, F268V, A245F, G265A, G252A, R336H, A259I, A133I, A123C, P130T, G113L, D208S, P280T, F268I, G181C, P280Y, R336E, P280G, G158C, N320Y, G140V, K349P, R292D, D208W, N320F, G265I, R336N, A164M, G206M, F268C, G158M, R292P, S191L, G158F, G252M, A245C, G229C, E235Q, G252V, A205M, G206F, R292Q, G181M, A126G, A243L, K349N, S283A, A246M, S279T, P186I, A246F, A246C, A223I, C97I, A164V, A245M, A171I, G357I, A126T, S283F, A123M, F194I, K117D, P226L, P130S, G140F, I276F, F194V, A243V, P1300, G181L, G229F, A259C, G113I, K117H, N320S, G119M, A239L, G206C, A114I, E235N, K349D, A242I, A161C, G294C, G119L, A126S. T321L, A193M, A223V, A122M, G113C, A242C, N201W, R292H, A275L, A242M, G357V, P186F, G294I, D208P, A259W, A243I, R310E, G140C, A242F, G265W, S183A, G109C, G113F, S191F, G265Y, S94D, E95K, A343K, A350K, F176D, N196D, H147D, H146D, H147E, H146E, T91E, R250E, T91D, R251E, R148D, H254E, A170K, R250K, R251K, H254D, A170R, H146K, H147K, K346E, T90D, R250D, T90E, A138K, R251D, H262K, A169K, H146R, I102E, T90K, A271K, K346R, H306E, D124E, K326E, A169R, D124R.
   64. Vaccine, method or composition according to any one of embodiments 1 to 63, comprising a PITP derivative, wherein the PITP derivative preferably comprises one or more, preferably one, two or three, especially one, of the following amino acid exchanges:
   T392F, F352L, T392I, M303F, A276Y, S365I, T392L, M303W, A276L, T392W, K363T, A361V, F286Y, A361I, K363F, M303Y, T392V, A361L, S365V, A270V, A276M, S365L, K363V, M312L, T252L, M387L, Y297F, A276F, G353T, A276W, K363S, T252R, L314I, T252I, A361F, L295I, L255I, A276I, K363L, S318W, V354L, K363I, A361T, S318I, A361M, G353L, T392Y, S365A, A270I, A361W, T252M, K363A, K363W, L319I, G353D, A276V, G353N, G353F, T252V, G353S, S318V, L255V, G364I, L394V, K363M, S318F, L390I, G353I, S335Y, G364L, S365M, T252Q, S365F, L330A, T252F, S318M, K363Y, G353V, S365G, S365W, K363G, T392M, V354F, A276T, G364W, K363C, P3171, S335C, P317L, P317F, G269C, S335A, P317V, G268C, S334V, S335V, G269V, S334C, A361C, G268I, G269F, G268L, S334I, G268V, G269L, S335G, S334A, G268F, S335T, P317C, P317M, S335L, A361Y, G269I, S335I, G268Y, K363P, G268W, S335M, K363H, F286I, G268M, G269M, G269W, G268A, S334G, S334L, G268P, G268T, G293F, F286L, T252C, S335N, T392C, K363N, S334F, M303I, K363Q, A376L, G364V, W245I, G269S, Y297C, S335Q, G358V, W245L, A376I, S334M, G358L, T252A, S318C, S335H, Y297L, S365C, M312I, G373L, Y297I, G358I, S335F, G268N, M303L, G269T, Y297V, P317A, G268Q, G268H, G293L, S334T, G305C, S318L, S335D, S335P, G364C, W245V, W245F, P317Y, T392A, F286V, S335W, A376V, G358C, K363D, M312F, G269N, S335R, A378I, A378C, P317W, G268K, G268R, G268D, A376C, G373V, M312V, M303V, G353C, S318A, G364F, G241C, G268E, G379I, G269P, F286C, L330F, G284V, W245C, A378V, L330I, G293I, G373C, G379L, S318Y, G358F, L394I, L330V, G373I, F352I, A361G, S335K, G246C, M387F, A378L, G241I, V354C, M387I, G269A, G308C, F352C, A276C, G293C, G269Q, K363R, G379V, F286W, W245M, G246V, A270C, G246L, M303C, G293V, G305A, Y297A, G345I, G241V; N388D, R248K, S336D, T372E, T249E, E316D, D348E, R382E, R382K, K265E, H327K, D338E, K333R, P386E, S336E, K341D, K341E, K341R. E385D, G268K, G268R, 0268D, K340R, G268E, K333D, K368R, G353D; H306I+S335R, H306L+S335R, H306I+S335K, H306C+S335R, H306C+S335R, H306I+S335K, H306L+S335K, H306L+S335K, H306L+S335R, H306V+S335R, H306I+S335R, H306V+S335R, H306C+S335K, H306F+S335K, H306V+S335K, H306V+S335K, H306F+S335R, H306F+S335R, H306W+S335R, H306F+S335K, H306M+S335R, H306W+S335K, H306W+S335R, H306M+S335K, H306A+S335R, H306A+S335R, H306W+S335K, H306A+S335K, H306Y+S335R, H306Y+S335R, H306Y+S335K, H306T+S335R, H306Y+S335K, H306T+S335R, H306T+S335K, H306S+S335R, H306T+S335K, H306S+S335K, S334V+S335R, H306S+S335K, S334V+S335K, S334I+S335R, S334C+S335R, H306P+S335R, M312I+S335R, S334I+S335K, S334V+S335R, S334V+S335K, H306P+S335K, S334C+S335R, S334A+S335R, M312F+S335R, M312L+S335R, S334C+S335K, H306R+Y381L, M312L+S335R, S250L+S335R, H306Q+S335R, H306N+S335K, S334C+S335K, S334A+S335K, H306P+S335K, H306Q+S335R, M312V+S335R, M312I+S335K, S334I+S335K, M312I+S335R, M312L+S335K, H306N+S335R, S334L+S335R, S334A+S335K, M312L+S335K, S2501+S335R, H306N+S335R, S250V+S335R, H306N+S335K, M312F+S335K, S250V+S335R, S250A+S335R, S334M+S335R, M312V+S335R, S250A+S335K, M312F+S335K, S250A+S335K, S250L+S335R, S334G+S335R, S250C+S335R, S250C+S335R, H306Q+S335K, S334G+S335R, Y254F+S335R, S334L+S335R, S2501+S335R, I309C+S335R, S250I+S335K, Y254C+S335R, S250L+S335K, M312V+S335K, S334G+S335K, S250L+H306R, S250V+H306R, H306R+Y381I, M312V+S335K, S2501+H306R, M312C+S335R, Y254C+S335R, S250V+S335K, Y254W+S335R, S334G+S335K, S250L+H306R, S250L+S335K, Y254W+S335R, S250V+S335K, S334L+S335K, I309C+S335R, I309C+S335K, Y254F+S335R, S250A+S335R, S250C+S335K, S250I+H306R, S250A+H306R, S250C+S335K, H306Q+S335K, Y254C+S335K, S334L+S335K, S250A+H306R, I309C+S335K, Y254W+S335K, Y254C+S335K, Y254I+S335R, H306R+Y381C, S250M+S335K, M312C+S335K, M312C+S335K, S250V+H306R, S250M+S335R, Y254V+S335R, Y254I+S335K, S250W+S335R, I259C+S335R, S334M+S335R, Y254A+S335R, Y254V+S335R, S250M+S335K, F251L+S35R, Y2541+H306R, I259C+S335K, Y254I+S335R, I259C+S335K, S250Y+S335K, Y254F+S335K, S250M+S335R, Y254F+H306R, Y254V+S335K, Y254V+H306R, S334T+S335K, S334Y+S335R, Y254A+S335R, S334M+S335K, Y254I+H306R, Y254M+S335K, Y254A+S335K, S334F+S335R, S334M+S335K, F251L+H306R, S334T+S335R, Y254I+S335K, Y254A+S335K, S334T+S335K, S250Y+H306R, Y254A+H306R, Y254M+S335K, F251L+S335K, I259C+S335R, F251L+H306R, Y254M+H306R, S334P+S335R, S334P+S335R, S334P+S335K, S334F+S335R, Y254M+S335R, H306I+S335C, H306L+S335C, H306L+S335C, H306I+S335C, H306V+S335C, H306C+S335C, H306I+S334V, H306V+S335C, H306C+S335C, H306I+S334V, H306L+S335V, H306M+S335C, H306I+S335G, H306I+S335G, H306I+S335A, H306I+S335A, H306F+S335C, H306F+S335C, H306L+S335V, H306I+S335T, H306L+S335A, H306C+S335A, H306I+S335V, H306I+S335V, H306L+S334V, H306L+S335A, H306C+S335V, H306L+S334C, H306I+S335I, H306I+S334A, H306L+S334C, H306L+S334V, H306F+S334V, H306C+S335A, H306F+S335V, H306C+S334V, H306C+S334V, H306I+S335L, H306C+S335V, H306L+S334I, H306M+S335C, H306I+S334C, H306I+S335I, H306W+S335C, H306V+S335A, H306I+S334C, H306I+S334I, H306L+S335I, H306V+S335A, H306C+S334C, H306L+S335G, H306C+S334C, H306L+S335T, H306I+S334I, H306V+S334V, H306I+S335L, H306M+S335V, H306V+S335V, H306V+S335V, H306I+S335M, H306L+S335T, H306V+S335I, H306C+S335G, H306L+S335L, H306C+S334I, H306L+S335Y, H306L+S335I, H306V+S334V, H306C+S335G, H306I+S334A, H306L+S334A, H306C+S335I, H306F+S335V, H306I+S335T, H306L+S335Y, H306C+S334I, H306L+S334I, H306V+S335G, H306V+S335G, H306A+S335C, H306A+S335C, H306M+S335A, H306C+S334A, H306V+S335I, H306L+S335G, H306L+S334A, H306C+S334A, H306I+S335Y, H306I+S335Y, H306W+S335C, H306F+S335A, H306F+S335A, H306F+S335I, H306I+S335N, H306L+S335L, H306I+S335M, H306I+S335N, H306V+S334I, H306V+S334C, H306V+S335L, H306C+S335L, H306C+S335L, H3061+A376L, H306F+S335T, H306L+S335M, H306F+S334V, S334V+S335C, S335C+Y381L, S335C+A376I, S334I+S335C, S334V+S335C, M312I+S335C, M312I+S335C, S335C+A376L, S334I+S335C, S335C+A376L, S335C+A378C, S335C+A376C, S335C+A376V, S334A+S335C, S335C+A378C, S334A+S335C, S335C+A3761, S334V+S335A, S335A+A376L, S335C+A376C, S335C+A376V, S335C+A378L, S334V+S335A, S335A+A376I, S334V+A376L, S334I+S335V, S335C+A378I, M312F+S335C, M312F+S335C, S334I+S335A, S335C+A378V, S335V+A376L, S334V+S335L, S335C+A378I, S334C+S335V, S334V+A376I, S334V+A376I, S335V+A376I, M312I+S334V, S334V+S335I, S335C+Y381I, M312V+S335C, S335C+A378L, S335A+Y381L, S334C+A376C, S334V+S335I, S335C+Y381V, M312L+S335C, S335C+A378V, S335V+A376L, S334I+S335V, S335C+Y381C, S335V+A376I, S334C+A376L, S334C+Y381L, M312V+S335C, S334V+S335L, S334C+A376I, S334C+A376C, S334V+Y381L, M312L+S335C, S335V+A376V, S3341+A376I, S335V+A376V, S335V+Y381L. Y254F+S335C, L310F+S335C, S3351+A376I, S334V+A376V, M312I+S335V, S334C+S335A, S3341+A376L, M312F+S335V, S335C+Y381A, L310F+S335C, S334G+S335C, S334G+S335C, S334V+A376V, S335C+A376M, S335C+A376M, S334A+S335I, S334V+A376L, M312C+S335C, S334C+S335A, S335A+A376V, S334A+S335V, S334A+S335V, Y254F+S335C, S335A+A376L, S335A+Y3811, S335A+A376I, S334L+S335C, S335A+A376V, M312C+S335C, D152F, D152L, D152I, D152V, D152M, D152W, D152Y, D152S, D152A, D152T, D152G, D152N, D152Q, D152P, D152H, D152K, D152R, D152C, D155Q, D155L, D155F, D155I, D155M, D155W, D155V, D155Y, D155A, D155N, D155T, D155S, D155P, D155G, D155H, D155R, D155K, D155C, D156L, D156F, D156I, D156W, D156V, D156A, D156Y, D156M, D156G. D156S. D156T, D156Q, D156N, D156P, D156H, D156K, D156R, D156C, H146R, H146K, H153R, H153K. Y63A, H146A, H153A, F74A, L141A, F81A, P72A, K144A, T143A, Y75A, W98A, D156A, R158A, S250W, S250F, S250D, S250E, S250K, S250R, F251A, Y254A, I259W, I259F, I259D, I259E, I259R, I259K, H306A, 1309W, I309F, I309E, I309D, I309R, I309K, L310W, L310F, L310D, L310E, L310R, L310K, M312W, M312F, M312D, M312E, M312R, M312K, S334W, S334F, S334D, S334E, S334R, S334K, S335W, S335F, S335D, S335E, S335K, S335R, M337W, M337F, M337D, M337E, M337K, M337R, A376W, A376F, A376E, A376D, A376R, A376K, F377, A378W, A378F, A378D, A378E, A378K, A378R, F380A, Y381A, D348I, D348L, D348M, D348F, D348V, D348Y, D348A, D348W, D348T, D348G, D348P, D348H, D348S, D348R, D348K, D348E, D348C, H306R, H306D, H306E, H306K, H307D, H307R, H307E, H307K, H327R, H327K, H327E, H327D, H302E, H302K, H302R, H302D, H215K, H215R, H215E, H215D, H223K, H223R, H223E, H223D, H403K, H403R, H403E, H403D.
   65. Vaccine, method or composition according to any one of embodiments 1 to 64, comprising a polypeptide with at least one P. acnes epitope of the polypeptide according to SEQ ID NO: 49, wherein the polypeptide with at least one P. acnes epitope of the polypeptide according to SEQ ID NO: 49 preferably comprises one or more, preferably one, two or three, especially one, of the following amino acid exchanges compared to SEQ ID NO: 49:
   A274Y, S26C, S26L, A274F, S26I, G119H, A274L, A274W, S26V, S26M, S212L, T108I, A134L, S26F, N130V, A229L, A274I, G194F, S26W, A291F, G181I, A229I, E278I, A175L, G181L, A152L, A274V, N130L, E278V, K46I, E278L, E278A, K46C, K46L, K93L, K93V, E164I, E278C, E164V, E278M, N130I, K46V, R221C, K93C, K46M, S51L, K93I, E278F, K93M, E164A, S51I, R221L, P209L, K46F, S51V, E164C, R221I, E164M, K93F, D137L, P209V, E164L, P59L, R221F, N130C, S51C, R221G, E278T, K46G, P59V, K46A, N130M, E278G, D137V, K46T, P209C, P59I, P209I, T144L, N130F, E164F, S51F, R221M, P209F, S51M, R221S, P209A, K93A, P209M, E164Y, D137C, R221A, P115C, P59F, S208A, K46S, P115V, A134I, P115A, S212V, N249L, E278S, D137F, D137M, P115L, S212I, R221V, N249V, D137I, E278Y, G194L, P59M, R221T, A188L, N249I, T144I, E164T, S208C, S208V, D137A, K46Y, K93G, G87I, E164G, K46P, P59C, A97V, S212C, G83L, K93T, N249C, A134V, G83V, T108V, T112L, T144V, A100L, G87V, E164W, E164S, A171L, A97L, A188F, K93W, T90I, A174I, K93Y, A111L, A971, N130A, G48I, E278W, R221Y, G135L, T112V, F197V, T144F, K93S, G194V, G87A, A188V, T144C, K46N, P115M, A1751, N30T, S51A, G194C, G48L, G104V, A174V, G158V, T90V, R221N, A202L, A39L, A134F, D137Y, G158I, A174L, P155V, A171V, S51T, F197I, T112C, D137W, G135V, D137G, P115G, T250C, S208L, A100I, T250L, T144M, T108C, P115F, G69L, T112F, G181F, A100F, S208I, G223V, T250V, N249M, A111V, N249Y, A111F, A188M, A111I, P209Y, A175V, P1551, N130G, S120V, A111C, A100C, A97F, G194M, G69I, E278Q, S208G, G181C, 087F, G48V, A97C, I205L, G194I, D137T, S51G, F197C, G223L, N249A, S51Y, P115S, A202I, P155C, S26A, G83M, S120C, D137S, E278N, G83F, G158C, P59Y, G87C, G87M, N130S, P115I, A202F, A100M, G83C, S212M, A39V, A1881, F197L, G181M, A134C, P115T, A174F, K93P, P209W, P209G, P59A, A39C, G104I, T250G, S208F, R221Q, G181A, G104C, T90C, K46Q, P209T, K46W, A171I, G158A, A134M, G48C, G194A, T90A, T90M, N130Y, N249F, A172L, A152V, A152I, P59W, R221P, G158F, T112M, G135M, G181V, G104L, S208T, N249W, A97M, G135F, K93Q, G42L, A111M, G104A, E278P, T90L, R221D, A175C, A188C, A291L, S212A, F123L, T250I, A174C, E164Q, P155L, A174M. G158M, A172V, E164N, A168L, T90F, A39F, G42V, I205F, A39M, N249G, A175F, G135C, A171F, S212F, K46D, A100Y, G421, P59G, G69A, E164P, G158L, G42C, A171C, T250M, T108L, K93N, A171M, G223I, A175M, T108M. A172I, S51P, A291I, A111Y, G20V, S120I, P59T, A58I, G223C, S208M, G48M, K46H, A62L, A43I, T250A, N130W, G194Y, R221H. D137P, N249S, G104F, A100V, F123I, A172C, T112I, G69F, P115Y, P59S, A100W, G104M, G69C, R221W, K93H, A204L, G20L, G223F, A168I, D28K, E275K, K77R, D17R, H76D, H75D, H76E, K77D, H75E, P294D, S293D, S293E, D286E, P294E, S292E, D285E, H76R, K77E, K289R, P295D, D285K, R179E, H75R, P295E, P294R, P294K, D286K, D285R, S293R, S293K, R180D, R180K, H183E, R179K. K289E, R180E, A98K, R179D, A99K, S292D, D286R, K290D, H75K, A99E, N256E, K290E, K290R, R239D, A199K, A200K, K255E, H183D, A99R, H235E, A98R, D102K, and A200R.
   66. Vaccine, composition, method or DsA1 and/or DsA2 derivative according to any one of embodiments 1 to 63 wherein the DsA1 domains are defined as follows: NSR: from S29 to I48, CSD1 from I49 to L130, SR1 from G131 to S147, CSD2 from A148 to L267, SR2 from A268 to T277, CSD3 from A278 to K323, the PT repeat region from P324 to T361, and CTR from S362 to F405 in the sequence of Q6A5X9 or any other functional DsA1 sequence comprising all these domains, especially the DsA1 sequences of FIG. 12 comprising all these domains;
   and/or wherein the DsA2 domains are defined as follows: NSR: from A72 to K92, CSD1 from 193 to L174, SR1 from S175 to S191, CSD2 from A192 to L311, SR2 from A312 to T321, CSD3 from A322 to E366, the PT repeat region from P367 to T420, and CTR from H421 to A463 in the sequence of Q6A5P9 or any other functional DsA2 sequence comprising all these domains, especially the DsA2 sequences of FIG. 12 comprising all these domains;
   and/or wherein the PITP domains are defined as follows: ENFD from A32 to R164; SR1 from E165 to K237, HbD from V238 to L396, SR2 (including C-terminal LPXT(G) motif (i.e. including LPXT, but not the G)) from S397 to T430, and hLAR from G431 to 1467 in the sequence of Q6A9N1 or any other functional PITP sequence comprising all these domains, especially the PITP sequences of FIG. 12 comprising all these domains.
   67. Pharmaceutical composition according to any one of claims 1 to 64 comprising in a liquid formulation or in a dry or frozen formulation which is reconstitutable to a liquid formulation: 0.01 mg/ml to 50 mg/ml, preferably 0.05 to 5 mg/ml, especially 0.1 to 1 mg/ml, polypeptide as defined above comprising at least one P. acnes epitope, 0.1 to 100 mM, preferably 0.5 to 50 mM, especially 0.1 to 10 mM, NaH₂PO₄, 0.1 to 100 mM, preferably 0.5 to 50 mM, especially 0.1 to 10 mM, Na₂HPO₄, 0.1 to 100 mM, preferably 0.5 to 50 mM, especially 0.1 to 10 mM, KH₂PO₄, 1 to 1000 mM, preferably 5 to 500 mM, especially 50 to 300 mM, NaCl, 0.1 to 100 mM, preferably 0.5 to 50 mM, especially 0.1 to 10 mM. KCl, and/or 0.01 to 1%, preferably 0.05 to 0.5%, especially 0.1 to 0.5%, aluminium adjuvant (aluminium hydroxide).
   68. Pharmaceutical composition according to any one of claims 1 to 67 having in a liquid formulation or in a dry or frozen formulation which is reconstitutable to a liquid formulation a pH of 4.5 to 7.4, preferably of 4.7 to 7.3, even more preferred of 5.0 to 7.3, especially of 5.3 to 7.2.
   69. Pharmaceutical composition comprising a P. acnes polypeptide or vaccine according to any one of embodiments 1 to 68, eliciting a cross-type reactivity at least against Types IA1, IA2, and II of P. acnes and/or eliciting a cross-type reactivity at least against Types IAI1, IA2, IB, II and III of P. acnes.
   70. Pharmaceutical composition comprising a P. acnes polypeptide or vaccine according to any one of embodiments 1 to 69, wherein the polypeptide is present in the composition at least partially in a form wherein it lacks an N-terminal methionine residue.
   71. Vaccine comprising at least two antigenic polypeptides with epitopes of P. acnes which are surface exposed, wherein preferably at least one epitope is an epitope of a dermatan sulfate-binding adhesin 1 of P. acnes (DsA1 polypeptide) which is surface exposed or an epitope of a putative iron-transport protein of P. acnes (PITP polypeptide) which is surface exposed, wherein DsA1 comprises from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Thr repeat containing region (“PT repeat region”), and a C-terminal region (“CTR”), and wherein the PITP polypeptide comprises from N- to C-terminus an extended neocarzinostatin family domain (“ENFD”), a first swapping region (“SR1”), a heme-binding domain (“HbD”), a second swapping region (“SR2”) including the C-terminal LPXTG motif, and a hydrophobic C-terminal region (“hLAR”).
   72. Vaccine comprising
at least one antigenic polypeptide with an epitope of a DsA1 polypeptide which is surface exposed or an epitope of a dermatan sulfate-binding adhesin 2 of P. acnes (DsA2 polypeptide) which is surface exposed,
   wherein DsA2 comprises from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), and, optionally, a Pro-Thr repeat containing region (“PT repeat region”), and a C-terminal region (“CTR”); and
at least one antigenic polypeptide with an epitope of a PITP polypeptide which is surface exposed.
   73. Vaccine comprising
at least one synthetic antigenic polypeptide with an epitope of a DsA1 polypeptide which is surface exposed or an epitope of a DsA2 polypeptide which is surface exposed; and
at least one synthetic antigenic polypeptide with an epitope of a PITP polypeptide of P. acnes which is surface exposed.
   74. Vaccine according to any one of embodiments 71 to 73 comprising the DsA1 polypeptide and/or the DsA2 polypeptide, and/or a fragment and/or a derivative of DsA1 or DsA2, wherein the fragment and/or the derivative of DsA1 or DsA2 comprises or consists at least of a CSD2 fragment, wherein the CSD2 fragment is preferably
a contiguous polypeptide sequence of phenylalanine 150 (F150) to leucine 184 (L184),
a contiguous polypeptide sequence of phenylalanine 150 (F150) to leucine 267 (L267), or
a contiguous polypeptide sequence of histidine 218 (H218) to leucine 267 (L267),
   with the proviso that the fragment or derivative does not comprise an NSR and a CTR, and wherein the amino acid numbering corresponds to the amino acid sequence Q6A5X9 in the UniProt database.
   75. Vaccine according to embodiment 74, wherein the fragment or the derivative of DsA1 or DsA2 comprises or consists at least of
a contiguous polypeptide sequence of phenylalanine 150 (F150) to isoleucine 193 (1193),
a contiguous polypeptide sequence of phenylalanine 150 (F150) to leucine 267 (L267), or
a contiguous polypeptide sequence of histidine 218 (H218) to leucine 267 (L267).
   76. Vaccine according to any one of embodiments 74 or 75, wherein the DsA1 or DsA2 fragment is
   (1) a contiguous polypeptide sequence of phenylalanine 150 (F150) to leucine 184 (L184), further extending one, two, three, or four amino acids at the N-terminus and/or one, two, three, four, five, six, seven, eight, nine, or ten amino acids at the C-terminus;
   (2) a contiguous polypeptide sequence of phenylalanine 150 (F150) to leucine 267 (L267), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus: and/or
   (3) a contiguous polypeptide sequence of histidine 218 (H218) to leucine 267 (L267) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus.
   77. Vaccine according to any one of embodiments 71 to 76, wherein the at least two antigenic polypeptides have a length of at least 35 amino acids, preferably a length of at least 40 amino acids, especially a length of at least 50 amino acids.
   78. Vaccine according to any one of embodiments 71 to 77 comprising a fragment or derivative of the PITP polypeptide, wherein the fragment and/or derivative of the PITP polypeptide is a PITP polypeptide wherein the hLAR is deleted, replaced by a hydrophilic C-terminal region, or partially deleted, wherein the partial deletion results in a loss of hLAR except the N-terminal 12 amino acids of hLAR, preferably except the N-terminal 11 amino acids of hLAR, especially except the N-terminal 10 amino acids of hLAR; or a fragment thereof or derivative thereof comprising at least amino acids corresponding to proline 34 to glutamic acid 73 or proline 94 to threonine 143 of ENFD or valine 238 to asparagine 393 of HbD in the amino acid sequence Q6A9N1 in the UniProt database.
   79. Vaccine according to embodiment 78, wherein the fragment or the derivative of the PITP polypeptide comprises or consists at least of
a contiguous polypeptide sequence of proline 34 to glutamic acid 73 of ENFD,
a contiguous polypeptide sequence of proline 94 to threonine 143 of ENFD, or
a contiguous polypeptide sequence of valine 238 to asparagine 393 of HbD.
   80. Vaccine according to any one of embodiments 74 to 79, wherein the vaccine further comprises a fragment or derivative of DsA1 or DsA2 comprising or consisting of at least an NSR, CSD1, SR1, CSD2, SR2, CSD3 and/or CTR from at least one other DsA1 and/or DsA2 from at least one other P. acnes strain than the strain from which the CSD2 fragment has been derived.
   81. Vaccine according to any one of embodiments 78 to 80, wherein the vaccine further comprises a fragment of ENFD and/or of HbD of the PITP polypeptide with a length of at least 8 amino acids, preferably at least 9 amino acids, more preferred at least 10 amino acids, more preferred at least 15 amino acids, even more preferred at least 35 amino acids, even more preferred a length of at least 40 amino acids, especially a length of at least 50 amino acids.
   82. Vaccine according to any one of embodiments 71 to 81 further comprising another P. acnes antigen or epitope, preferably an antigen or epitope selected from DsA1, DsA2 and/or PITP and/or a DsA1 epitope, a DsA2 epitope and/or a PITP epitope.
   83. Vaccine according to any one of embodiments 71 to 82 further comprising an epitope containing fragment of another P. acnes polypeptide.
   84. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP according to any one of embodiments 71 to 83 for use in a therapeutic treatment, preferably for use in the treatment or prevention of P. acnes-associated infections, especially selected from the group consisting of acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes.
   85. Composition comprising at least two antigenic polypeptides with epitopes of P. acnes which are surface exposed, preferably a composition comprising DsA1 and/or DsA2 and/or PITP, and/or a fragment and/or a derivative of DsA1 and/or DsA2 and/or PITP according to any one of embodiments 71 to 83 for use in the treatment or prevention of P. acnes-associated infections in a human patient suffering from P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes.
   86. Pharmaceutical formulation, especially vaccine, according to any one of embodiments 1 to 85, comprising a PITP polypeptide and/or a porphyrin-binding PITP fragment and/or a porphyrin-binding PITP derivative, further comprising a porphyin molecule and/or a porphyrinogen molecule and/or a porphyrinogen or porphyrin degradation product bound to the PITP and/or a porphyrin-binding PITP fragment and/or a porphyrin-binding PITP derivative, wherein the porphyin molecule and/or porphyrinogen molecule and/or porphyrinogen or porphyrin degradation product preferably contains a metal ion in complex form, wherein the metal ion is preferably a Fe, Co, Zn, Mg, Se, or Cu ion, especially a Fe²⁺ or a Fe³⁺ ion.
   87. Pharmaceutical formulation, especially vaccine, according to any one of embodiments 1 to 86, comprising a PITP polypeptide and/or a porphyrin-binding PITP fragment and/or a porphyrin-binding PITP derivative, further comprising a porphyin molecule and/or a porphyrinogen molecule and/or a porphyrinogen or porphyrin degradation product bound to the PITP and/or a porphyrin-binding PITP fragment and/or a porphyrin-binding PITP derivative, wherein the porphyin molecule and/or porphyrinogen molecule and/or porphyrinogen or porphyrin degradation product is selected from the group hemin, protoprophyrin IX, coproporphyrin I, II and/or III, especially coproporphyrin III; pentacarboxyporphyrin, uroporphyrin, heptacarboxyporphyrin, hexacarboxyporphyrin, especially as form I or III; nitro-porphyrins, especially 5,10,15,20-tetraphenylporphyrin (TPP), 5,10,15,20-tetra(4′-fluorophenyl)porphyrin (TpFPP), 5,10,15,20-tetra(4′-chlorophenyl)porphyrin (TpCIPP), 5,10,15,20-tetra(4′-bromophenyl)porphyrin (TpBrPP), biliverdin, 5,10-diphenyl-15,20-di(N-methylpyridinium-4-yl)porphyrin (Di-cis-Py+), PORF-1 to PORF-34, porphine, octaethylporphyrin, tetraphenylporphyrin, verteporfin, ascoproporphyrin, hydroxymethylbilane (HMB), mesoporphyrin IX, 7-carboxyporphyrin (7P), 6-carboxylporphyrin (6P), heme A, heme B, heme C, heme O, heme I, heme m, heme D. S heme; uroporphyrinogen III, chlorophylls, especially chlorophyll a, chlorophyll b, and bacteriochlorophylls.
   88. Vaccine or pharmaceutical formulation as defined in any one of embodiments 1 to 87 for use in the treatment or prevention of P. acnes-associated infections, comprising a polypeptide comprising an epitope of DsA1 and/or DsA2 and/or PITP, wherein the epitope is selected from the group consisting of R32-141, Q38-K51, R32-K51, T43-K51, Q38-K51. R87-K90+T43-K51, R87-K90+T117-I132 and R87-K90+S234-G250, R87-K90+L246-A260, R87-K90+A256-E270, R87-K90+R266-T277, T117-I132, T117-A127, V128-I132, A144-N157, H146-A160, A148-N157. A156-A170, K166-L180, A176-T190, P186-A198, N181-E191, I216-F224, I216-D225, A226-A24G, S234-G250, I251-I263, I264-P271, P236-G250, L246-A260, A256-E27G, S234-G250, I251-I263, I251-L267, A268-L280, R266-T277, T285-R286+I216-F224, T285-R286+I1264-P271, T285-R286+V289-K296, T285-R286+V289-K296, T285-R286+A144-N157, A310-D313+T285-R286, T285-D290, T285-D290+V291-T300, T285-D290+A301-E307, V291-T300, A301-E307, T285-T300, A301-E307, R286-D290+V291-T300, R286-D290+A301-E307, R286-T300, V289-K296, A310-D313+I216-F224, A310-D313+I264-P271, A310-D313+T285-R286, A310-D313+R286-D290, A310-D313+V289-K296, A310-D313+V289-K296, A310-D313+T285-T300, A310-D313+A144-N157, A310-D313+T285-R286, A310-D313+T285-D290, A310-D313+T293-E307, T285-D290, V291-T300, T293-E307, A301-E307 of DsA1; L152-Q166, G190-P230, I199-D208, A218-I237, P230-Q244, I231-A270, H254-A270, A271-S279, A271-R310, L311-T321, L311-K323, V333-Q347, A218-P230, I231-I237, H254-H262, Q256-H262, E261-D269, D269-S279, K313-K323, of DsA2; D79-T90, E73-D85, R43-150, P68-Y75, P86-E92, I39-G45, Y84-D89, F81-D89. D79-T90, T37-E44, E73-W98, E73-F81, D89-T90, P72-F81, A129-F138, D120-Q134, F111-D120, F132-G147, D152-E165, R115-F123, D120-K128, P131-F138, N181-E191, T143-T159, P116-T124, P131-D137, P131-D137, T175-C231, Q198-K203, P179-K185, G200-Q210, K174-A188, K174-K185, P201-Q209, P183-P201, P183-K191, K185-P195, R164-S180, E165-S180, K185-S190. V193-N202, V193-G200, K203-P208, R216-T225, R216-R224, P173-K191, K197-K203, P168-T175, K185-K203, R164-K174, T175-V193, S250-N261, D287-S300, K340-V347, D338-F352, D338-D348, S285-P288+G305-L314, S285-P288+H306-L314, S285-P288+T342-T351, S285-P288+D338-D348, D287-S300, T342-T351, D338-D348, H306-L314, G305-L314, G364-K375, R382-E399, V367-G373, A383-L390, T342-T351, M387-T395, E385-T392, V401-V410, N404-A409, G416-L427, L396-V410, T406-1415, D417-G424, V407-D418, V407-V414, K421-V429, S419-T430, D408-1415, T406-V414, of PITP, preferably wherein the epitope comprises at least one additional amino acid residue at the N- or C-terminus of the DsA1, DsA2, or PITP sequence, especially wherein the epitope comprises at least two additional amino acid residue at the N- or C-terminus of the DsA1, DsA2, or PITP sequence; wherein the polypeptide is preferably covalently linked to a carrier molecule or embedded in a scaffold molecule, especially a carrier polypeptide.
   89. Vaccine or pharmaceutical formulation as defined in any one of embodiments 1 to 87 for use in the treatment or prevention of P. acnes-associated infections, comprising a polypeptide comprising an epitope of DsA1 and/or DsA2 and/or PITP, wherein the polypeptide is a DsA1/DsA2/PITP derivative with the motif K[NYI] starting at position 5 or 6 of the polypeptide and/or having on DNA level the sequence AADUAU or AAVAUU at this coding position.
   90. Vaccine or polypeptide according to any one of embodiments 1 to 89, wherein the DsA1 and/or DsA2 and/or PITP, and/or a fragment or derivative of DsA1 or DsA2 or PITP is provided as DNA or RNA encoding an antigen or an epitope containing fragment or derivative thereof, preferably as mRNA vaccine, especially an mRNA vaccine formulated with cationic polymers, with an mRNA molecule with the following structure: 5′UTR-signal peptide-encoded antigen or epitope-3′UTR; or as a vector-based vaccine, especially with an adenovirus vector, an adeno-associated virus vector, a vesicular stomatitis virus vector, a Newcastle Disease virus vector, an alphavirus vector, a baculovirus vector, or a retrovirus vector; and/or wherein the coding sequence on DNA level has the initial coding DNA sequence ATGGTG; especially wherein the mRNA comprises a sequence as transcribed from SEQ ID NOs: 67 to 76.
   91. Pharmaceutical formulation comprising a vaccine or polypeptide according to any one of embodiments 1 to 90 comprising the polypeptide as defined in any one of these claims, i.e. a DsA1 and/or DsA2 and/or PITP, and/or a fragment and/or a derivative of DsA1 and/or DsA2 and/or PITP according to any one of embodiments 1 to 90 or mixtures thereof; and a pharmaceutically acceptable excipient or carrier.
   92. Method of treatment or prevention of P. acnes-associated infections in a human patient comprising administration of an effective amount of a DsA1 and/or DsA2 and/or PITP, and/or a fragment and/or a derivative of DsA1 and/or DsA2 and/or PITP according to any one of embodiments 1 to 91 or mixtures thereof in an effective amount to a patient in need thereof.
   93. Method of treatment or prevention of P. acnes-associated infections in a human patient suffering from P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes comprising administration of an effective amount of a DsA1 and/or DsA2 and/or PITP, and/or a fragment and/or a derivative of DsA1 and/or DsA2 and/or PITP according to any one of embodiments 1 to 91 or mixtures thereof in an effective amount to a patient in need thereof.
   94. Method according to claim 93, wherein administration is performed by intradermal, subcutaneous (s.c.), parenteral, intramuscular (i.m.), mucosal, transcutaneous or topical administration, preferably by intradermal, or intramuscular administration, especially by a syringe or by microneedeling devices.
   95. Method of production of the polypeptides, vaccines and formulations according to any one of embodiments 1 to 91, wherein the polypeptides comprising at least one P. acnes epitope as defined in these claims are expressed in a host cell, extracted and purified from these host cells, and, optionally, formulated and finished to a pharmaceutical formulation, especially a vaccine for use in the treatment or prevention of P. acnes-associated infections in a human patient.
   96. Vaccine according to any one of embodiments 1 to 91 comprising a polypeptide
   (a) comprising at least an epitope-containing fragment or derivative of CSD1 of DsA1 and at least an epitope-containing fragment or derivative of CSD2 of DsA2: and/or
   (b) comprising at least a polypeptide stretch comprising a CSD1 fragment or derivative of CSD1 of DsA1 of at least 30 amino acid residues, preferably at least 40 amino acid residues, and at least a polypeptide stretch comprising a CSD2 fragment or derivative of CSD2 of DsA1 of at least 30 amino acid residues, preferably at least 40 amino acid residues;
   (c) comprising at least
a contiguous polypeptide sequence of phenylalanine 150 (F150) to leucine 184 (L184),
a contiguous polypeptide sequence of phenylalanine 150 (F150) to leucine 267 (L267). or
a contiguous polypeptide sequence of histidine 218 (H218) to leucine 267 (L267) of DsA1, and
a contiguous polypeptide sequence of phenylalanine 194 (F194) to leucine 228 (L228),
a contiguous polypeptide sequence of phenylalanine 194 (F194) to leucine 311 (L311), or
a contiguous polypeptide sequence of histidine 262 (H262) to leucine 311 (L311) of DsA2,
   especially wherein the polypeptide comprises an amino acid exchange at one or more of C53, C319 and C321 of DsA1, and C97 and C363 of DsA2, if present in the polypeptide, preferably one or more of amino acid exchanges C53S, C319S and C321P of DsA1 and C97S and C363S of DsA2 and/or wherein in the fragment or derivative of DsA1 or DsA2 at least 5 PT repeats, preferably at least 10 PT repeats, especially at least 15 PT repeats, are deleted compared to a naturally occurring wild type DsA1/DsA2 polypeptide (“native DsA1/DsA2”), and wherein preferably at least one, more preferred at least two, more preferred at least three, even more preferred at least four, especially five, PT repeat(s) is/are present.
   97. Vaccine according to any one of claims 1 to 96, wherein the polypeptide further comprises a PITP polypeptide or a fragment or derivative of PITP comprising a polypeptide stretch of at least 30 amino acid residues comprising at least an epitope of PITP, preferably wherein the PITP polypeptide or a fragment or derivative of PITP comprises at least
a contiguous polypeptide sequence of proline 34 to glutamic acid 73 of ENFD or a PITP derivative thereof.
a contiguous polypeptide sequence of proline 94 to threonine 143 of ENFD or a PITP derivative thereof, or
a contiguous polypeptide sequence of valine 238 to asparagine 393 of HbD or a PITP derivative thereof;
   especially wherein the PITP derivative comprises an amino acid exchange exchange at positions C231 and C402.
   98. Method of production of the polypeptides, vaccines and formulations according to any one of embodiments 1 to 91, wherein the polypeptides comprising at least one P. acnes epitope as defined in these claims are expressed or produced as an mRNA in a host cell, extracted and purified from these host cells, and, optionally, formulated and finished to a pharmaceutical formulation, especially a vaccine for use in the treatment or prevention of P. acnes-associated infections in a human patient.
   99. Method according to embodiment 98, wherein the host cell is an eukaryotic or prokaryotic host cell, preferably a prokaryotic host cell, especially E. coli or L. lactis; or a host cell which is able to glycosylate the expressed polypeptides as defined in any one of embodiments 1 to 96.
   100. Method according to embodiment 98 or 99, wherein the host cell is naturally unable to synthesize porphyrins and wherein preferably porphyrins are added to the culture, to the lysis buffer, and/or to the purified protein.
   101. Method according to any one of embodiments 98 to 100, wherein the polypeptides of SEQ ID NOs:1 to 66 or encoded by SEQ ID NOs:67 to 76 are expressed in the host cells.

Therefore, the present invention also discloses the following embodiments, specifically drawn i.a to the aspect of the present invention wherein in a product, preferably a pharmaceutical product, especially a vaccine, a fusion/shuffeled polypeptide with at least one polypeptide stretch of DsA1 and at least one polypeptide stretch of DsA2 is provided:

1. A polypeptide comprising at least one polypeptide stretch of Dermatan sulfate-binding adhesin 1 of P. acnes (DsA1) and at least one polypeptide stretch of Dermatan sulfate-binding adhesin 2 of P. acnes (DsA2), said DsA1 and DsA2 comprising from N- to C-terminus an N-terminal region, a first conserved sub-domain (“CSD1”), a first swapping region (“SR1”), a second conserved sub-domain (“CSD2”), a second swapping region (“SR2”), a third conserved sub-domain (“CSD3”); and, optionally a Pro-Leu repeat containing region (“PT repeat region”), and a C-terminal region;
wherein the polypeptide comprises at least an epitope of CSD1, CSD2 or CSD3 of DsA1 and at least an epitope CSD1, CSD2 or CSD3 of DsA2, preferably wherein the polypeptide comprises at least CSD1, CSD2 or CSD3 of DsA1 and CSD1, CSD2 or CSD3 of DsA2.
2. A polypeptide comprising at least one polypeptide stretch of Dermatan sulfate-binding adhesin 1 of P. acnes (DsA1) and at least one polypeptide stretch of Dermatan sulfate-binding adhesin 2 of P. acnes (DsA2), said DsA1 and DsA2 comprising from N- to C-terminus an N-terminal region, a first conserved sub-domain (“CSD1”), a first swapping region (“SR1”), a second conserved sub-domain (“CSD2”), a second swapping region (“SR2”), a third conserved sub-domain (“CSD3”); and, optionally, a Pro-Leu repeat containing region (“PT repeat region”), and a C-terminal region;
wherein the polypeptide stretch of DsA1 and DsA2 has independently a length of at least 20 amino acid residues.
3. A polypeptide according to embodiment 1 or 2, wherein at least either the polypeptide stretch of DsA1 or DsA2 comprises or consist of a DsA1 or DsA2 epitope: or wherein the at least two polypeptide stretches form a DsA1 or DsA2 epitope.
4. A polypeptide according to any one of embodiments 1 to 3, comprising at least two of CSD1, CSD2 and CSD3 of DsA1 or DsA2.
5. A polypeptide according to any one of embodiments 1 to 4, having at least two polypeptide stretches of DsA1 or DsA2, wherein CSD1, CSD2 and/or CSD3 of DsA1 or DsA2 are connected by SR1 and/or SR2, wherein SR1 and SR2 are of DsA1 or DsA2 or a hybrid SR, wherein the hybrid SR consist of amino acid residues being present either in a DsA1 or in a DsA2 SR.
6. A polypeptide according to any one of embodiments 1 to 5, wherein the polypeptide stretch comprises or consists at least of a CSD2 fragment, wherein the CSD2 fragment is preferably
a contiguous polypeptide sequence of phenylalanine 150 (F150) to leucine 184 (L184),
a contiguous polypeptide sequence of phenylalanine 150 (F150) to leucine 267 (L267), or
a contiguous polypeptide sequence of histidine 218 (H218) to leucine 267 (L267),
   and wherein the amino acid numbering corresponds to the amino acid sequence Q6A5X9 in the UniProt database.
   7. A polypeptide according to any one of embodiments 1 to 6, wherein the polypeptide stretch comprises or consists at least of
a contiguous polypeptide sequence of phenylalanine 150 (F150) to isoleucine 193 (1193),
a contiguous polypeptide sequence of phenylalanine 150 (F150) to leucine 267 (L267), or
a contiguous polypeptide sequence of histidine 218 (H218) to leucine 267 (L267).
   8. A polypeptide according to any one of embodiments 1 to 7, wherein the polypeptide stretch is
   (1) a contiguous polypeptide sequence of phenylalanine 150 (F150) to leucine 184 (L184), further extending one, two, three, or four amino acids at the N-terminus and/or one, two, three, four, five, six, seven, eight, nine, or ten amino acids at the C-terminus;
   (2) a contiguous polypeptide sequence of phenylalanine 150 (F150) to leucine 267 (L267), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus;
   (3) a contiguous polypeptide sequence of histidine 218 (H218) to leucine 267 (L267) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus;
   (4) a contiguous polypeptide sequence of phenylalanine 150 (F150) to leucine 267 (L267), further extending one, two, three, or four amino acids at the N-terminus and/or one, two, three, four, five, six, seven, eight, nine, or ten amino acids at the C-terminus;
   (5) a contiguous polypeptide sequence of alanine 148 (F148) to leucine 271 (L271), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus;
   (6) a contiguous polypeptide sequence of histidine 146 (H146) to threonine 277 (T277) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus;
   (7) a contiguous polypeptide sequence of leucine 238 (L238) to leucine 272 (L272), further extending one, two, three, or four amino acids at the N-terminus and/or one, two, three, four, five, six, seven, eight, nine, or ten amino acids at the C-terminus;
   (8) a contiguous polypeptide sequence of leucine 145 (L145) to alanine 201 (A201), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus; and/or
   (9) a contiguous polypeptide sequence of leucine 145 (L145) to leucine 280 (L280) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus.
   9. A polypeptide according to any one of embodiments 1 to 6, wherein the polypeptide stretch has a length of at least 35 amino acids, preferably a length of at least 40 amino acids, especially a length of at least 50 amino acids.
   10. A polypeptide according to any one of embodiments 1 to 8, which comprises at least one additional polypeptide with an amino acid sequence containing or consisting of at least one additional P. acnes antigen or P. acnes epitope which is not a DsA1 or DsA2 antigen or epitope, preferably at least one additional amino acid sequence containing at least the putative iron-transport protein (PITP) polypeptide of P. acnes or at least one epitope of PITP polypeptide of P. acnes, wherein the PITP polypeptide comprises from N- to C-terminus an extended neocarzinostatin family domain (“ENFD”), a first swapping region (“SR1”), a heme-binding domain (“HbD”), a second swapping region (“SR2”) including the C-terminal LPXT(G) motif, and a hydrophobic C-terminal region (“hLAR”).
   11. A polypeptide according to embodiment 10 wherein the additional polypeptide is the PITP polypeptide or a fragment or derivative thereof, wherein the fragment or derivative thereof comprises a sequence comprising at least one of ENFD, SR1, HbD, SR2, preferably selected from ENFD and HbD or an epitope containing fragment thereof, more preferred a porphyrin-binding domain thereof, especially wherein the fragment of the PITP polypeptide is selected from the group of the polypeptides consisting of the amino acids A32 to T430, A32 to G426, A32 to Q198, A32 to T143, A32 to K400, A32 to T159, A32 to I177, A32 to Q204, A32 to G234. A32 to R164. A32 to S391, A32 to P179, A32 to R158, A32 to G147, A32 to E73 and P94 to G147; P34 to T430, P34 to G426, P34 to Q198, P34 to T143, P34 to K400, P34 to T159, P34 to I177, P34 to Q204, P34 to G234, P34 to R164, P34 to S391, P34 to P179, P34 to R158, P34 to G147, P34 to E73 and P94 to G147; S240 to S391, A32 to D44I, A32 to I440, A32 to E439, A32 to D438, A32 to S437. A32 to S436, A32 to P435, A32 to G434, A32 to E433, A32 to A432, A32 to G431; P34 to D441, P34 to I440, P34 to E439, P34 to D438, P34 to S437, P34 to S436, P34 to P435, P34 to 0434, P34 to E433, P34 to A432, P34 to 0431; S240 to D441. S240 to I440, S240 to E439, S240 to D438, S240 to S437. S240 to S436, S240 to P435, S240 to G434, S240 to E433, S240 to A432, S240 to G431; A32 to T430, A32 to V429, A32 to P428, A32 to L427, A32 to G426, A32 to R425, A32 to G424, A32 to A423, A32 to G422, A32 to K421, A32 to G420, A32 to S419, A32 to D418, A32 to D417, A32 to G416, A32 to 1415, A32 to V414, A32 to K413, A32 to G412, A32 to T411, A32 to V410, A32 to A409, A32 to D408, A32 to V407, A32 to T406, A32 to V405, A32 to N404, A32 to H403, A32 to C402, A32 to V401, A32 to K400. A32 to E399, A32 to A398. A32 to S397. A32 to L396, A32 to T395, A32 to L394, A32 to N393, A32 to T392: P34 to T430, P34 to V429, P34 to P428, P34 to L427, P34 to G426, P34 to R425, P34 to G424, P34 to A423, P34 to G422, P34 to K421, P34 to G420, P34 to S419, P34 to D418, P34 to D417, P34 to G416, P34 to 1415, P34 to V414, P34 to K413, P34 to G412, P34 to T411, P34 to V410, P34 to A409, P34 to D408, P34 to V407, P34 to T406, P34 to V405, P34 to N404, P34 to H403, P34 to C402, P34 to V401, P34 to K400, P34 to E399, P34 to A398, P34 to S397, P34 to L396, P34 to T395, P34 to L394, P34 to N393, P34 to T392; G172 to T430, G172 to V401, G172 to K400, G172 to L396, G172 to N393, A199 to T430, A199 to V401, A199 to K400, A199 to L396, A199 to N393, H223 to T430, H223 to V401, H223 to K400, H223 to L396, H223 to N393, T232 to T430, T232 to V401, I232 to K400, T232 to L396, T232 to N393, G234 to T430, G234 to V401, G234 to K400, G234 to L396, G234 to N393, V238 to T430, V238 to V401, V238 to K400, V238 to L396, V238 to N393, S240 to T430, S240 to V429, S240 to P428, S240 to L427, S240 to 0426, S240 to R425, S240 to G424, S240 to A423, S240 to G422, S240 to K421, S240 to G42G, S240 to S419, S240 to D418, S240 to D417, S240 to G416, S240 to 1415, S240 to V414, S240 to K413, S240 to G412, S240 to T411, S240 to V41G, S240 to A409, S240 to D408, S240 to V407, S240 to T406, S240 to V405, S240 to N404, S240 to H403. S240 to C402. S240 to V401, S240 to K40G, S240 to E399, S240 to A398, S240 to S397, S240 to L396, S240 to T395, S240 to L394, S240 to N393, S240 to T392; 39I-48I, 40P-49T, 41V-50I, 42G-51S, 43R-52G, 44E-53K, 68P-77N, 69A-78S, 70S-79D, 71V-80K. 72P-81F, 73E-82Y, 74F-83G, 75Y-84Y, 76G-85D, 77N-86P, 78S-87S, 79D-88K, 80K-89D, 81F-90T, 82Y-91T, 83G-92E, 84Y-93S, 85D-94P, 86P-95S, 87S-96T, 88K-971, 89D-98W, 90T-99V, 91T-100Y, 92E-101T, 93S-102P, 94P-103S, 95S-104Q, 96T-105K, 97I-106A, 98W-107I, 99V-108G, 100Y-109S, 101T-110R, 102P-111F, 103S-112A, 104Q-113Q, 105K-114G, 106A-115R, 107I-116P, 108G-117M, 109S-118N, 110R-119N, 111F-120D, I12A-I21G, 1251-134Q, I26T-135G, I27M-136K, I28K-137D, 144K-153H, 145A-154S, 146H-155D, 147G-156D, 148V-157T, 149G-158R, 150K-159T, 151T-160P, 152D-161V, 153H-162T, 154S-163Y, 155D-164R, 156D-165E, 157T-166A, 158R-167T, 159T-168P, 160P-169A, 161V-170P, 162T-171T, 163Y-172G, 164R-173P, 165E-174K, 166A-175T, 167T-176P, 168P-1771, 169A-178A, 170P-179P, 171T-180S, 172G-181K, 173P-182Q, 174K-183P, 175T-184S, 176P-185K, 1771-186Q, 178A-187A, 179P-188A, 180S-189P, 181K-190S, 182Q-191K, 183P-192Q, 184S-193V, 185K-194K, 186Q-195P, 187A-196S, 188A-197K, 189P-198Q, 190S-199A, 191K-200G, 192Q-201P, 193V-202N, 194K-203K, 195P-204Q, 196S-205S, 197K-206T, 198Q-207T, 199A-208P, 200G-209Q, 201P-210Q, 202N-211K, 203K-212T, 204Q-213A, 205S-214E, 206T-215H, 207T-216R, 208P-217S, 209Q-218Q, 210Q-219T, 211K-220P, 212T-221A, 213A-222A, 214E-223H, 215H-224R, 216R-225T, 217S-226M, 218Q-227T, 219T-228K, 220P-229Q, 221A-230V. 222A-231C, 223H-232T, 224R-233I, 225T-234G, 226M-235A, 227T-236S, 228K-237K, 229Q-238V, 230V-239T, 231C-240S, 232T-241G, 233I-242S, 266L-275S, 267S-276A, 268G-277F, 282T-291K, 334S-343N, 353G-3621, 354V-363K, 355S-364G, 356V-365S, 357S-366P, 358G-367V, 359N-368K, 377F-386P, 378A-387M, 379G-388N, 380F-389P, 396L-405V, 397S-406T, 398A-407V, 399E-408D, 400K-409A, 401V-410V, 402C-411T, 403H-412G, 404N-413K, 405V-414V, 406T-4151, 407V-416G, 408D-417D, 409A-418D, 410V-419S, 411T-420G, 412G-421K, 413K-422G, 414V-423A, 415I-424G, 416G-425R, 417D-426G, 418D-427L, 419S-428P, 4200-429V, 421K-430T, 422G-431G, 423A-432A, 424G-433E, 425R-434G, 426G-435P, 427L-436S, 428P-437S, 429V-438D, 430T-439E, 431G-440I, 432A-441D, 433E-442L, 434G-443G, 435P-444I, 436S-445V, and 437S-446G; 139-K153, P68-G121, I125-D137, K144-S242, L266-F277, T282-K291, S334-N343, G353-K368, F377-P389, and L396-G446, especially a fragment or derivative comprising or consisting of the fragments selected from the group consisting of the PITP fragments A32 to R164, A32 to Q198, A32 to T143, A32 to V148, A32 to T171, P34 to R164, A32 to T159, A32 to I177, A32 to Q204, A32 to G234, A32 to K400, A32 to S391, V238 to K400. A199 to T430, V238 to T395, G234 to K400. H223 to K400, T232 to V401, V238 to T392, V238 to N393, V238 to L394, V238 to T395, V238 to L396, T232 to T430, G172 to K400, and G172 to G234, and fragments thereof comprising at least of a PITP epitope, according to the amino acid numbering in the amino acid sequence Q6A9N1 of the UniProt database.
   12. A polypeptide according to any one of embodiments 1 to 11, wherein the at least one polypeptide stretch comprises a fragment of DsA1 or DsA2 being
   (1) a contiguous polypeptide sequence of phenylalanine 150 (F150) to leucine 184 (L184), optionally further extending one, two, three, or four amino acids at the N-terminus and/or one, two, three, four, five, six, seven, eight, nine, or ten amino acids at the C-terminus;
   (2) a contiguous polypeptide sequence of phenylalanine 150 (F150) to leucine 267 (L267), optionally further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus;
   (3) a contiguous polypeptide sequence of histidine 218 (H218) to leucine 267 (L267) optionally further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus.
   13. Vaccine comprising a polypeptide according to any one of embodiments 1 to 12, and, optionally, a pharmaceutically acceptable excipient.
   14. Vaccine according to embodiment 13, wherein at least one of the polypeptide stretches is a fragment or a derivative of DsA1 or DsA2 and comprises a fragment of DsA1 or DsA2 with at least 20 contiguous amino acids and wherein in the fragment or derivative at least 5 PT repeats, preferably at least 10 PT repeats, especially at least 15 PT repeats, are deleted compared to a naturally occurring wild type DsA1/DsA2 polypeptide (“native DsA1/DsA2”), and wherein preferably at least one, more preferred at least two, more preferred at least three, even more preferred at least four, especially five, PT repeat(s) is/are present.
   15. Vaccine according to embodiments 13 or 14, wherein the polypeptide comprises a derivative of DsA1 and/or DsA2, wherein the derivative comprises an NSR, CSD1, SR1, CSD2, SR2, CSD3 and/or CTR from at least one other DsA1 and/or DsA2 or from at least one other P. acnes strain than the strain from which the CSD2 fragment.
   16. Vaccine according to any one of embodiments 13 to 15, wherein the polypeptide lacks the NSR, CTR, and/or the LPXTG motif.
   17. Vaccine according to any one of embodiments 13 to 16 further comprising another P. acnes antigen or epitope, preferably an antigen selected from DsA2 and PITP and/or a DsA2 epitope and/or an PITP epitope.
   18. Vaccine according to any one of embodiments 13 to 17 or a polypeptide according to any one of embodiments 1 to 12, further comprising an epitope containing fragment of another P. acnes polypeptide, preferably an epitope containing fragment of DsA2 and PITP.
   19. Vaccine according to any one of embodiments 13 to 18, wherein the vaccine is protective against development of a pathological condition induced by P. acnes invasion, preferably wherein the vaccine is formulated for administration to a human subject, e.g. a child, an adolescent or adult subject.
   20. Vaccine according to any one of embodiments 1 to 19, wherein the DsA1 and/or DsA2 and/or PITP, and/or a fragment or derivative of DsA1 or DsA2 or PITP is provided as DNA or RNA encoding an antigen or an epitope containing fragment or derivative thereof, preferably as mRNA vaccine, especially an mRNA vaccine formulated with cationic polymers, with an mRNA molecule with the following structure: 5′UTR-signal peptide-encoded antigen or epitope-3′UTR; or as a vector-based vaccine, especially with an adenovirus vector, an adeno-associated virus vector, a vesicular stomatitis virus vector, a Newcastle Disease virus vector, an alphavirus vector, a baculovirus vector, or a retrovirus vector; and/or wherein the coding sequence on DNA level has the initial coding DNA sequence ATGGTG; especially wherein the mRNA comprises a sequence as transcribed from SEQ ID NOs: 67 to 76.
   21. Vaccine according to any one of embodiments 13 to 20, wherein the vaccine is formulated for use in a treatment regimen involving both, a prime and boost immunization, preferably wherein the same formulation is used for the prime and boost administration.
   22. Vaccine according to any one of embodiments 13 to 21, wherein the vaccine is formulated with an adjuvant, preferably with an adjuvant selected from the group consisting of mineral salts, preferably alum (potassium aluminum sulfate), aluminum hydroxide, aluminum phosphate, aluminum oxohydroxide, aluminum hydroxyphosphate, calcium phosphate, cerium nitrate, zinc sulfate, colloidal iron hydroxide, and/or calcium chloride; oil-in-water emulsions, liposomes, TLR agonists, Monophosphoryl Lipid A, saponins, phospholipids, emulsion-based adjuvants, aluminium hydroxide gel, solid phase adsorbents, nanospheres and encapsulating materials such as liposomes: high molecular weight adjuvants, preferably toxins, toxoids or any mutant cross-reactive material of the toxin from tetanus, diphtheria, pertussis. Pseudomonas species, E. coli, Staphylococcus species, and Streptococcus species. Such toxins or toxoids may be tetanus toxoid, pertussis toxoid, cholera toxoid, E. coli LT, E. coli ST, and exotoxin A from Pseudomonas aeruginosa; bacterial outer membrane proteins such as outer membrane protein complex c (OMPC), porins, transferrin binding proteins, pneumolysin, pneumococcal surface protein A (PspA), pneumococcal adhesin protein (PsaA), C. difficile enterotoxin (toxin A) and cytotoxin (toxin B) or Haemophilus influenzae protein D, other pharmaceutically acceptable polypeptide carriers, such as ovalbumin, keyhole limpet hemocyanin (KLH), bovine serum albumin (BSA) or purified protein derivative of tuberculin (PPD); cationic peptides, CpG oligonucleotide, squalene-based adjuvants, preferably MF59; cytokines, such as IL-1 and IL-2; or formulated as virus-like particles or nanoparticles: or combinations thereof.
   23. Vaccine according to any one of embodiments 13 to 22, wherein the vaccine is formulated for intradermal, subcutaneous (s.c.), parenteral, preferably intramuscular (i.m.), mucosal, transcutaneous or topical administration.
   24. Vaccine according to any one of embodiments 13 to 23, wherein the vaccine comprises 0.1 μg to 5 mg, preferably 0.5 to 1000 μg, more preferred 1 to 500 μg, even more preferred 5 to 300 μg, especially 10 to 200 μg, of the polypeptide according to any one of embodiments 1 to 12 per dose.
   25. Vaccine according to any one of embodiments 13 to 24, wherein the vaccine contains a composite immunogen and is engineered by linking of one or more antigen(s) of P. acnes and/or one or more cross-reactive, especially cross-type-reactive, or otherwise immunologically relevant epitope(s) of P. acnes to each other, preferably wherein the immunogen comprises or consists of at least 2 (e.g. 2, 3, 4, 5, 6, 7, 8, 9, or 10) antigen(s) and/or immunologically relevant epitope(s) thereof, especially wherein the antigens and/or epitopes are cross-reactive, especially cross-type-reactive.
   26. Vaccine according to any one of embodiments 13 to 25, wherein the vaccine contains a composite immunogen comprising or consisting of at least two (e.g., 2, 3, 4, 5, 6 7, 8, 9, or 10) antigen(s) of P. acnes and/or epitope(s) thereof linked to an adjuvant, wherein at least one antigen or epitope is immunologically relevant.
   27. Vaccine according to any one of embodiments 13 to 26, wherein the vaccine comprises a variant of an antigen of P. acnes.
   28. Vaccine according to any one of embodiments 13 to 27, wherein the vaccine comprises a variant of an an epitope of P. acnes.
   29. Vaccine according to any one of embodiments 13 to 28, wherein the vaccine comprises a polypeptide with at least about 90% amino acid sequence identity, preferably at least 95%, more preferably at least 98%, more preferably at least 99% or at least 99.5%, to the amino acid sequence of a naturally occurring P. acnes antigen; or a polypeptide of a P. acnes epitope, wherein at least 1, 2, 3, 4, up to 5 point mutations are contained in the amino acid sequence compared to the naturally occurring epitope.
   30. Vaccine according to any one of embodiments 13 to 29, wherein the vaccine comprises a variant, preferably a DsA1 and/or DsA2 protein wherein one or more amino acid residues are added, or deleted, at the N- or C-terminus, as well as within one or more internal domains, especially a variant comprising additional amino acids at the N-terminal and/or at the C-terminal end, to prolong an antigen sequence, e.g. to prolong a sequence of an epitope or epitopic region within a protein by at least one amino acid residue, preferably by less than 3 amino acids, specifically less than 5, or else less than 10 amino acids.
   31. Vaccine according to any one of embodiments 13 to 30, wherein the vaccine comprises a derivative, especially as a fusion protein, wherein a PITP fragment containing a PITP epitope is prolonged by additional amino acid residues of another polypeptide or protein, preferably by one or more immunologically relevant epitopes, especially wherein the derivative comprises a His-tag at the N- or at the C-terminus comprising at least 4, preferably at least 5, especially at least 6 histidine residues.
   32. Vaccine according to any one of embodiments 13 to 31, wherein the vaccine comprises a fragment or derivative of DsA1 or DsA2, wherein DsA1 and DsA2 comprise from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Leu repeat containing region (“PT repeat region”), and a C-terminal region (“CTR”), wherein fragment or derivative comprises or consists at least of (1) a contiguous polypeptide sequence of phenylalanine 150 (F150) to leucine 184 (L184). (2) a contiguous polypeptide sequence of phenylalanine 150 (F150) to leucine 267 (L267), or (3) a contiguous polypeptide sequence of histidine 218 (H218) to leucine 267 (L267), with the proviso that the fragment or derivative does not comprise the NSR and the CTR, said fragment or derivative optionally comprising one or more amino acid exchanges, wherein one or more amino acid residues are changed to a cysteine (Cys, C) residue to further stabilise the fragment or derivative by the ability to form disulfide bonds.
   33. Vaccine according to any one of embodiments 13 to 32, wherein the fragment or derivative has a length of at least 20, preferably at least 30, more preferred at least 60 amino acids, even more preferred a length of at least 80 amino acids, especially a length of at least 100 amino acids.
   34. Vaccine according to any one of embodiments 13 to 33, wherein the fragment or derivative has a length of less than 400 amino acids, preferably less than 350 amino acids, especially less than 300 amino acid residues.
   35. Vaccine according to any one of embodiments 13 to 33, wherein the fragment or derivative has a length of less than 250 amino acids, preferably less than 200 amino acids, especially less than 150 amino acid residues.
   36. Vaccine according to any one of embodiments 13 to 35, wherein two or more amino acid residues in a fragment or derivative are changed to a cysteine (Cys, C) residue to further stabilise the fragment or derivative by the ability to form disulfide bonds, preferably (with respect to the DsA1 sequence (which is also applicable to the DsA2 sequence; FIG. 12A)) containing at least one of the amino acid pairs in the following group wherein the native amino acid is exchanged to cysteine: PHE150-GLY185, LEU167-ALA199, ASN157-ALA192, LYS166-ALA199, ALA153-GLY 185, ASP164-ALA199, VAL154-ALA179, PHE150-LEU184, VAL154-ALA188, ALA153-ALA188, ALA179-GLU191, THR178-GLU191, ALA161-ALA195, ILE175-ALA195, ILE175-GLU191, LEU158-ALA176, ASN157-ALA188. ASN157-ALA179. ASP164-GLU196, VAL154-LEU180, ALA153-LYS189, ASN157-LYS189, ALA161-ALA192, LEU167-ALA198, LEU158-ALA179, ALA160-ALA192, LYS165-ALA199, LYS166-GLN200; VAL205-ALA260, ILE 232-ARG266. SER235-ILE263, SER235-AR0262, ILE232-LEU267, PRO236-ILE263, ALA231-ARG266, ASN239-ILE259, ILE232-ILE263, VAL209-PRO236, ALA201-ALA256, SER235-ILE259, VAL228-ARG266, ALA202-ALA256, ALA201-VAL257, GLY206-VAL243, GLY208-ILE264, GLY208-ILE263, VAL205-ASN239, LEU238-ILE259, GLY208-ALA260, ALA201-LYS253, ALA202-VAL243, LEU204-ALA260, VAL205-ALA256, VAL205-ILE259, SER235-ARG266, ASN239-ALA260, VAL209-ILE263, or VAL209-ASN239.
   37. Vaccine according to any one of embodiments 13 to 36, wherein a cysteine in the DsA1, DsA2 or PITP or fragment or derivative thereof is exchanged, preferably wherein the cysteine is exchanged with serine, proline, alanine, threonine, asparagine, glutamine, valine, isoleucine, leucine, phenylalanine, tyrosine and glycine, more preferred with serine, proline, alanine, threonine, asparagine, glutamine, valine, isoleucine, leucine, especially with serine, proline, alanine, threonine, asparagine, and glutamine.
   38. Vaccine according to any one of embodiments 13 to 38, wherein the fragment or derivative has a length of length of 35 to 350 amino acids, preferably of 40 to 300 amino acids, more preferred of 50 to 250 amino acid residues, more preferred of 60 to 200 amino acids, more preferred of 70 to 150 amino acids, especially of 90 to 130 amino acid residues.
   39. Vaccine according to any one of embodiments 13 to 37, wherein the derivative comprises at least one polypeptide stretch of DsA1 and at least one polypeptide stretch of DsA2, preferably wherein the derivative comprises at least CSD1, CSD2 or CSD3 of DsA1 and at least CSD1, CSD2 or CSD3 of DsA2.
   40. Vaccine according to any one of embodiments 13 to 39, wherein the derivative comprises at least one fragment comprising at least CSD1, CSD2 or CSD3 of DsA1 and at least one fragment comprising at least CSD1, CSD2 or CSD3 of DsA2 and wherein the at least two fragments are interconnected by
an SR1 or SR2 of DsA1 or DsA2 adjacent to the at least CSD1, CSD2 or CSD3 of DsA1 and at least CSD1, CSD2 or CSD3 of DsA2; or
an amino acid sequence consisting of an N-terminal part of an SR1 or SR2 of DsA1 or DsA2, adjacent to the at least CSD1, CSD2 or CSD3 of DsA1 and at least CSD1, CSD2 or CSD3 of DsA2, and a C-terminal part of a different SR1 or SR2 of DsA1 or DsA2, adjacent to the at least CSD1, CSD2 or CSD3 of DsA1 and at least CSD1, CSD2 or CSD3 of DsA2, wherein the total number of amino acid residues the amino acid sequence consisting of the of the N- and C-terminal part is identical to the SR1 or SR2 of DsA1 or DsA2.
   41. Vaccine according to any one of embodiments 13 to 40, wherein the derivative comprises at least one fragment comprising at least CSD1, CSD2 or CSD3 of DsA1 and at least one fragment comprising at least CSD1, CSD2 or CSD3 of DsA2, wherein the fragment of DsA1 and DsA2 has independently a length of at least 20 amino acid residues, preferably at least 30, more preferred at least 40, especially at least 50, amino acid residues
   42. A polypeptide according to any one of embodiments 1 to 13 or a vaccine according to any one of embodiments 13 to 41 for use in a therapeutic and prophylactic treatment, preferably for use in the treatment or prevention of P. acnes-associated infections, especially selected from the group consisting of acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, artherosclerosis, prostethic cancer and a medical implant biofilm infection by P. acnes.
   43. A polypeptide according to any one of embodiments 1 to 13 or a vaccine according to any one of embodiments 13 to 41 for use in the treatment or prevention of P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes.
   44. A polypeptide or a vaccine for use according to embodiment 42 or 43, wherein the human patient is suffering from P. acnes-associated infections and pathological conditions associated with at least two P. acnes MLST phylotypes, and/or at least two different CC types of P. acnes and/or at least two ribotypes of P. acnes, as determined by a surface binding measurement employing flow cytometry and in a bactericidal killing assay using immune serum raised against such vaccine antigen.
   45. A polypeptide or vaccine for use according to any one of embodiments 42 to 44, wherein the human patient is suffering from P. acnes-associated infections and pathological conditions associated with at least two or three genetically different types of P. acnes strains, preferably against at least one Type I and at least one Type II or III strains; or from at least one Type H and at least one Type I or III strains; or from at least one Type III and at least one Type I or II strains; or from at least one Type I, at least one Type II, and at least one Type III strain.
   46. A polypeptide or vaccine for use according to any one of embodiments 42 to 45, wherein the human patient is suffering from P. acnes-associated infections and pathological conditions associated with two or more of P. acnes strains selected from the group consisting of Type IA1, Type IA2, Type IB, Type IC, Type H and Type III strains, or Type I-Ia, I-lb, I-2 and II, especially Type IAI, IA2, and II, as defined according to the MLST typing schemes.
   47. A polypeptide or vaccine for use according to any one of embodiments 42 to 46, wherein the human patient is suffering from P. acnes-associated infections and pathological conditions associated with two or more of P. acnes strains selected from the group of the ribotypes as determined according to 16S ribosomal sequence differences.
   48. A polypeptide or vaccine for use according to any one of embodiments 42 to 47, wherein the human patient is suffering from P. acnes-associated infections and pathological conditions associated with two or more phylotypes IA1, IA2, IB1, IB2, IB3, IC, II and III determined by the analysis of a single locus (SLST).
   49. A polypeptide or vaccine for use according to any one of embodiments 42 to 48, wherein the human patient is suffering from P. acnes-associated infections and pathological conditions associated with at least two different phylogenetic types, more preferably three, even more preferably four, five or six phylogenetic types of the following group of strains: NCTC737. KPA171202 (DSMZ, German Collection of Microorganisms and Cell Cultures. Braunschweig, Germany), SK137, HL005PA1, HL005PA4, HL013PA1, HL030PA1, HL043PA1, HL053PA1, HL053PA2, HL050PA1, HL050PA2, HL060PA1, HL110PA4 (BEI, Biodefense and Emerging Infections Research Resources Repository, Manassas, VA), P.acn31 and Asn12 (McDowell et al. 2012) and IAI 008, IAI031, IAI034, IAI035, IAI038, IAI040, IAI042, IAI045, IAI041 (Charite Berlin, Pro-Implant foundation).
   50. A polypeptide or vaccine for use according to any one of embodiments 42 to 49 for use in protection against infection with P. acnes.
   51. A polypeptide or vaccine for use according to any one of embodiments 42 to 50 for use in prevention or reducing development of a P. acnes-associated indication.
   52. A polypeptide or vaccine for use according to any one of embodiments 42 to 51, wherein the vaccine repeatedly administered to a human patient, preferably by at least 2 or 3 administrations, or at least 4, 5, or even more repeated administrations.
   53. A polypeptide or vaccine for use according to any one of embodiments 42 to 52, wherein the vaccine is used in one or more treatment cycles, each involving at least two, or three or four consecutive administrations within a period of 1 year in intervals of at least 1 or 2 weeks, especially wherein the treatment cycles are repeated at least any of 1×, 2×, 3×, or 4×, even more times, e.g. within a period of 5 years.
   54. A polypeptide or vaccine for use according to any one of embodiments 42 to 53, wherein the vaccine provokes an immune response, including the production of antibodies and/or cytokines and/or the activation of cytotoxic T cells, B-cells, antigen presenting cells, helper T cells, dendritic cells and/or other cellular responses in an individual upon administration.
   55. A polypeptide or vaccine for use according to any one of embodiments 42 to 54, wherein the vaccine is administered to a patient with a skin or other affected organ or tissue condition in which the strains from any of Type I, II, or III P. acnes, or a combination of at least two phylotypes of Type I, II, and III, or of at least two ribotypes of P. acnes are involved as pathogenic factors, or to a healthy individual that may be susceptible to an infection by any of the P. acnes strains, especially Type IA1, IA2, and II.
   56. A polypeptide or vaccine for use according to any one of embodiments 42 to 55, wherein the vaccine is administered to a patient by intradermal, transdermal or a subcutaneous administration, preferably by intradermal injection applicators, microneedles, transdermal laser devices, topical, skin patch or other suitable skin-adapted application device.
   57. A polypeptide or vaccine for use according to any one of embodiments 42 to 55, wherein the vaccine is administered by oral or parenteral administration such as intramuscular, intranodal, intranasal, mucosal, intrapulmonary, vaginal, rectal or other suitable routes.
   58. Fragment or derivative of DsA1, DsA2 or PITP as defined in any one of embodiments 1 to 57, preferably a fragment or derivative according to SEQ ID NOs: 5 to 7, 9 to 0.12, 14 to 20, and 29 to 50.
   59. Pharmaceutical composition comprising a fragment or derivative according to embodiment 58.
   60. Pharmaceutical composition according to embodiment 59, further comprising a pharmaceutically acceptable carrier.
   61. A method for producing a vaccine, wherein a polypeptide according to any one of embodiments to or as defined in any one of embodiments to 41 is mixed with a pharmaceutically acceptable excipient or carrier and, optionally, finished into a pharmaceutically administrable vaccine dose, preferably in a dosage unit suitable for administration to an individual, especially a human individual.
   62. Polypeptide, vaccine, method or composition according to any one of embodiments 1 to 61 wherein the polypeptide, comprises:
   (a) a CSD2 fragment of DsA1 or DsA2 within A148 to L267 of DsA1 or A192 to L311 of DsA2, at least comprising a contiguous polypeptide sequence of F150 to L184 of DsA1 or F194 to L228 of DsA2,
   (b) a CSD2 derivative of (a) with one or more, preferably one, two or three, especially one, of the following amino acid exchanges: A149T, D152N, D152S, V155I, V163I, K166H, K166P, A168T, K169R, T171A, V173M, A176V, A183T, T190F, T190I, E191K, A192L, A192F, A198G, A199T, A202T, V205I, G206N, G206S, G206R, G206Q, K212Q, A214T, I219V, I219A, S223A, D225N, V233I, S235F, N239S, L255I, V2571, Q258S, I259L, R262H, I264V, D265K in DsA1 and T193A, N196D, N196S, I199V, I207V, H210K, H210P, A212T, R213K, T215A, V217M, V220A, T227A, F234T, F234I, E235K, L236A, L236F, A242G, A243T, A246T, I249V, R250G, R250S, R250N, G250Q, Q256K, A258T, V2631, V263A, A267S, D269N, I277V, S279F, S283N, I299L, I301V, S302Q, L303I, H306R, V308I, K309D in DsA2
   (c) a CSD1 fragment of DsA1 or DsA2 within I49 to L130 of DsA1 or 193 to L174 of DsA2, at least comprising a contiguous polypeptide sequence of 20 amino acid residues, especially T117 to A127 of DsA1 or T161 to LA171 of DsA2,
   (d) a CSD1 derivative of (c) with one or more, preferably one, two or three, especially one, of the following amino acid exchanges: D50N, D50S, K51E, K51A, C53Y, D55K, V57I, A61L, A65G, A68V, L71M, F76L, S78A, V82M, V82A, L84M, P86R, P86S, K90R, K90A, K94A, V97T, L99T, I100L, K104R, K106E, A107V, I109V, G110A, A111S, L113V, G114S, L116V, T117A, K120A, I121V, R123H, A124T, V1281 in DsA1 and S94D, S94N, E95A, E95K, C97Y, K99D, I101V, L105A, G109A, V112A, L115M, L120F, A122S, A126M, A126V, L128M, P130R, P130S, A134K, A134R, A138K, T141V, T143L, I144L, R148K, K150E, V151A, V153I, A154G, S155A, L157V, G158S, V160L, A161 T, A164K, V165I, H167R, A168T, I172V in DsA2
   (e) a CSD3 fragment of DsA1 or DsA2 within A278 to K323 of DsA1 or A322 to E366 of DsA2, at least comprising a contiguous polypeptide sequence of R266 to T277 or R286 to K296 of DsA1 or V333 to Q347 of DsA2
   (f) a CSD3 derivative with one or more, preferably one, two or three, especially one, of the following amino acid exchanges:
   M281V, N283D, T285A, R286Q, V289A, V2911, I292R, T293N, A294T, D295K, D295Q, K296E, I298V, K299A, T300V, T300I, A301Y,—between 301 and 302 D, E302K, E305K, K306A, A310T, D313G, K316Q, S320T, C321—, P322L, K323D, K323E in DsA1 and V325M, D327N, A329T, R330Q, V333A, I335V, R3361, N337T, T338A, Q339K, Q339D, E340K, I342V. A343K, V344T, V3441, Y345A, K346E, K349E, A350K. T354A, G357D, Q360K, T364S, P365L, E366D, E366K, in DsA2.
   63. Polypeptide, vaccine, method or composition according to any one of embodiments 1 to 62 wherein the DsA1 stretch and/or DsA2 stretch comprises one or more, preferably one, two or three, especially one, of the following amino acid exchanges:
   DsA1: A301Y, G206L, G206F, A192L, G206R, G206W, G206Q, G206K, A301F, A301L, G206I, A301W, A301I, G206E, G206M, G208L, G206V, A192M, A256L, A192I, A68I, G206N, A68V, I259L, A161L, V205I, G206T, G206H, A68L, G206Y, A2561, G206A, 68M, A192F, G110A, N239L, G206C, A301V, E305L, A93I, A93L, S223A, E305I, E305V, E305A, K120V, K120L, N157I, K120C, E305C, E305M, N239I, K120I, R248C, N239V, E191I, E305F, S78I, K120M, E191L, R248L, H146V, E191V, H146I, S78L, S78V, N157V, H146C, K120F, R248I, R248M, D164L, P236V, P236L, H146L, H146F, D164I, H146M, P86L, R248G, N239C, N157C, E305T, E191C, S78C, R248F, G221L, E305G, P236F, T171L, P86V, R248S, N157L, K120A, H146A, P86I, N157F, S235A, R248A, E191M, N157M, A124I, P236A, S78F, P236C, S78M, A161I, N239M, T171I, E191F, S235V, P86F, H146G, S223I, E305S, D164F, E305Y, P236M, S223L, N239F, H146Y, P236I, G221V, D164C, K120G, D164M, S235I, R248V, A124V, S235L, S223V, S147L, S235C, P86M, G208I, D164A, S1471, N239A, K120T, T1171, G110L, R248Y, P182I, A198L, A124L, A215L, A127L, A66L, P86C, G221F, R248T, T171F, P182V, S147V, P271V, T171V, G110V, P271A, E191A, A149L, K120Y, G114I, K120S, S235F, A201I, G131V, A201V, R248N, A66V, A202I, G221C, G114V, A229L, H146T, A124F, A127F, T117V, S223C, A215F, A1271, P182C, A215V, G162L, G162I, G208F, A201L, A161F, N157A, A198V, E191Y, A161V, S223F, G221M, R248W, G131C, A202V, N157T, S147F, N157Y, G114A, T171C, H146W, S78T, A149I, G250L, N239T, A149V, G208C, K120P, G250V, S235G, S223M, D164V, N239G, T171M, E191G, S78A, D164Y, P271I, G110F, G110C, D164W, P236Y, A124C, A66F, S147M, G75L, E305N, S147C, G221I. A127M, P182L. G221A, G110M, A66C, E305Q, P271F, A215M, P236W, K120W, H146P, A161C, A229I, S78G, G114M, P86A, E305W, V154L, G185V, G75I, S223Y, G75V, R248Q, T277V, E191S, A229F, T277G, G131I, N239S, P271C, V154I, A66M, D1640, G208A, P236T, R248D, D164T, A127C, A215I, A161M, P86W, A201F, C53L, T117F, G131A, T117C, A318L, F150L, G208M, P271M, A149C, G208V, G114C, G162F, S223W, A1981, N157G, A202L, P2360, G185I, A198F, A149M, P86Y, E191W, N157W, I263L, S235T, E305P, E305R, A201C, A201M, F150I, G96I, G114F, G221Y, A199L. D164S. A124M. A179I, A202F, H146S. A231L, C53I, I263V, G162M, K120Q, N239Y, 096L, G75C, N157S. T277C, T117A, A199V, G162C, P271Y, G250C, A127Y, A661, T117L, G185C, A229C, R248P, A1441, T117M, P86T, A260L, G185A, E191Q, G131L, T277I, G250I, G131M, T277A, A127V, F150V, A318I, S78W, S147Y, A198M, P860, A215C, A701, S235M, A179V, A202M, A144V, A202C, G208W, T171Y, S147W, G208Y, G250F, A195L, K120H, A127W, A66Y, S223G, K120N, S78Y, E191N, A198C, P271G, V154F, A70V, H146N, A229M, A134L, P86S, S78Q, A199I, G75M, A68C, E191T, A89L, G96A, D164P, C53V, S78P, G185L, A1490, A149F, P182A;
   DsA2: A161L, A161I, A126I, A126V, A126L, S175G, G190H, A161T, S183T, R336I, A126F, A161F, A329L, A161V, S283L, A205L, G109A. A329I, G265F, A161W, A232I, S175W. R336L, A300I, K349I, A161M, A300L, G109L. G109I, A246L, S183M, A223L, E235V, G252L, A329M, A126W, S183L, A259L, A329V, A329F, S183I, A242L, G109F, R336V, K117I, R336C, K117C, K349V, R336A, R336F, K117L, R336M, E235I, R310A, K349L, K117V, R310V, K349C, N201I, R310I, R310C, K349F, K349M, K117M, R292C, N201V, R336G, R292L, K117F, E235C, P130L, E235A, D208L, P280L, E235L, R292M, R336T, P280V, R2921, N201L, E235M, P130I, K117A, K349A, P130V, R292G, R310F, R336S, R336Y, N201C, K117G, R310L, R310M, R310G, R336W, D208V, R292F, P130F, K349Y, K117T, R310T, P280C, R292S, N201F, R310S, T215L, N201M, S279C, R292A, S279L, P280F, D208C, S175V, A126M, P280A, S279A, P130C, S279V, E235F, P186C, P130M, K349T, A126C, K349G, N320V, K117S, N3201, S283V, A168I, N320L, S175I, E235Y, P186V, P280I, P186A, P280M, G265L, D208M, P186L, K117Y, S283I, A205I, D208F, A168L, T215I, R292V, A133L, R336P, R310Y, A168V, S279F, S183V, A164I, S279I, S175L, D208A, K349S, E235T, S175C, A122I, D208I, A126Y, A123L, K117P, N320C, R292T, S283C, E235W, G252I, T215V, R292Y, R336Q, S175A, A123I, E2350, A205V, A171V, A133V, A245I, S183C, P226V, R310P, G181I, P226I, A168C, A246I, A171L, N201A, S175M, G158I, A164L, T215C, A164C, P280W, A133C, G119I, T215F, A245V, N320M, A122V, A123V, G206L, N320A, G265V, A122L, A193L, S191V, R310W, 0252F, G181V, E235S, R292N, P130W, G119V, G265C, S279M, A259V, A259F, A168F, A168M, A246V, P226C, R292W, G158V, P130Y, G206V, P1860, C97L, N201Y, A171C, 0265M, G140L, K117N, G140I, D208G, A193V, G181F, K349W, S279G, A245L, A205F, G158A, A193I, K117W, A122C, R310D, G294V, P186T, S175F, S191C, N201T, K349Q, R310N, A242V, T215M, D208Y, T321C, G252C, 1276L, A259M, A171F, N201G, G119F, D208T, P186M, G229V, 0294L, S191I, P186S, G119C, G229I, P130A. A205C, A171M, T321V, A193C, F268L, A133M, A123F, G357A, G109V, K117Q, N320G, R310Q, T321G, S283M, P186Y, G229A, R336D, N201S, S183F, F194L, F268V, A245F, G265A, G252A, R336H, A259I, A133I, A123C, P130T, G113L, D208S. P280T, F268I, G181C, P280Y. R336E, P280G, G158C, N320Y. G140V, K349P, R292D, D208W, N320F, G265I, R336N, A164M, 0206M, F268C, G158M, R292P, S191L, G158F, G252M, A245C, G229C, E235Q, G252V, A205M, G206F, R292Q, G181M, A126G, A243L, K349N, S283A, A246M, S279T, P186I, A246F, A246C, A2231, C97I, A164V, A245M, A171I, G357I, A126T, S283F, A123M, F194I, K117D, P226L, P130S, 0140F, I276F, F194V, A243V, P130G, G181L, G229F, A259C, G113I, K117H. N320S, G119M, A239L, G206C, A114I, E235N, K349D, A242I, A161C, G294C, G119L, A126S, T321L, A193M, A223V, A122M, G113C, A242C, N201W, R292H, A275L, A242M, G357V, P186F, G294I, D208P, A259W, A243I, R310E, G140C, A242F, G265W, S183A, G109C, G113F, S191F, G265Y, S94D, E95K, A343K, A350K, F176D, N196D, H147D, H146D, H147E, H146E, T91E, R250E, T911D, R251E, R148D, H254E, A170K, R250K, R251K, H254D, A170R, H1146K, H147K, K346E, T90D, R250D, T90E, A138K, R251D. H262K, A169K, H146R, I102E, T90K, A271K, K346R, H306E, D124E, K326E, A169R, D124R.
   64. Polypeptide, vaccine, method or composition according to any one of embodiments 1 to 63, further comprising at least one additional amino acid sequence containing at least the PITP of P. acnes or at least one epitope of PITP polypeptide of P. acnes, (a PITP derivative”), wherein the PITP derivative preferably comprises one or more, preferably one, two or three, especially one, of the following amino acid exchanges:
   T392F, F352L, T392I, M303F, A276Y. S365I, T392L, M303W, A276L, T392W, K363T, A361V, F286Y, A361I, K363F, M303Y, T392V, A361L, S365V, A270V. A276M, S365L, K363V, M312L, T252L, M387L, Y297F, A276F, G353T, A276W, K363S, T252R, L314I, T252I, A361F, L295I, L255I, A276I, K363L, S318W, V354L, K363I, A361T, S318I, A361M, G353L, T392Y, S365A, A270I, A361W, T252M, K363A, K363W, L319I, G353D, A276V, G353N, G353F, T252V, G353S, S318V, L255V, G364I, L394V, K363M, S318F, L390I, G353I, S335Y, G364L, S365M, T252Q, S365F, L330A, T252F, S318M, K363Y, G353V, S365G, S365W, K363G, T392M, V354F, A276T, G364W, K363C, P3171, S335C, P317L, P317F, G269C, S335A, P317V, 0268C, S334V, S335V, G269V, S334C, A361C, G268I, G269F, G268L, S334I, G268V, 0269L, S335G, S334A, 0268F, S335T, P317C, P317M, S335L, A361Y, G269I, S335I, G268Y, K363P, G268W, S335M, K363H, F286I, G268M, G269M, G269W, 0268A, S334G, S334L, G268P, 0268T, 0293F, F286L, T252C, S335N, T392C, K363N, S334F, M303I, K363Q, A376L, G364V, W2451, 0269S, Y297C, S335Q, G358V, W245L, A376I, S334M, G358L, T252A, S318C, S335H, Y297L, S365C, M312I, G373L, Y297I, G358I, S335F, G268N, M303L, G269T, Y297V, P317A, G268Q, G268H, G293L, S334T, G305C, S318L, S335D, S335P, G364C, W245V, W245F, P317Y, T392A, F286V, S335W, A376V, G358C, K363D, M312F, G269N, S335R, A378I, A378C, P317W, G268K, G268R, G268D, A376C, G373V, M312V, M303V, G353C, S318A, G364F, G241C, G268E, G379I, G269P, F286C, L330F, G284V, W245C, A378V, L330I, G293I, G373C, G379L, S318Y, G358F, L394I, L330V, G373I, F352I, A361G, S335K, G246C, M387F, A378L, G241I, V354C, M387I, G269A, G308C, F352C, A276C, G293C, G269Q, K363R, 0379V, F286W, W245M, G246V, A270C, G246L, M303C, G293V, G305A, Y297A, G345I, G241V; N388D, R248K, S336D, T372E, T249E, E316D, D348E, R382E, R382K, K265E, H327K, D338E, K333R, P386E, S336E, K341D, K341E, K341R. E385D, G268K, G268R, G268D, K340R, G268E. K333D, K368R, G353D; H306I+S335R, H306L+S335R, H306I+S335K, H306C+S335R, H306C+S335R, H306I+S335K, H306L+S335K, H306L+S335K, H306L+S335R, H306V+S335R, H306I+S335R, H306V+S335R, H306C+S335K, H306F+S335K, H306V+S335K, H306V+S335K, H306F+S335R, H306F+S335R, H306W+S335R, H306F+S335K, H306M+S335R, H306W+S335K, H306W+S335R, H306M+S335K, H306A+S335R, H306A+S335R, H306W+S335K, H306A+S335K, H306Y+S335R, H306Y+S335R, H306Y+S335K, H306T+S335R, H306Y+S335K, H306T+S335R, H306T+S335K, H306S+S335R, H306T+S335K, H306S+S335K, S334V+S335R, H306S+S335K, S334V+S335K, S334I+S335R, S334C+S335R, H306P+S335R, M312I+S335R, S334I+S335K, S334V+S335R, S334V+S335K, H306P+S335K, S334C+S335R, S334A+S335R, M312F+S335R, M312L+S335R, S334C+S335K, H306R+Y381L, M312L+S335R, S250L+S335R, H306Q+S335R, H306N+S335K, S334C+S335K, S334A+S335K, H306P+S335K, H306Q+S335R, M312V+S335R, M312I+S335K, S334I+S335K, M312I+S335R, M312L+S335K, H306N+S335R, S334L+S335R, S334A+S335K, M312L+S335K, S2501+S335R, H306N+S335R, S250V+S335R, H306N+S335K, M312F+S335K, S250V+S335R, S250A+S335R, S334M+S335R, M312V+S335R, S250A+S335K, M312F+S335K, S250A+S335K, S250L+S335R, S334G+S335R, S250C+S335R, S250C+S335R, H306Q+S335K, S334G+S335R, Y254F+S335R. S334L+S335R, S250I+S335R, I309C+S335R, S2501+S335K, Y254C+S335R, S250L+S335K, M312V+S335K, S334G+S335K, S250L+H306R, S250V+H306R, H306R+Y3811, M312V+S335K, S250I+H306R, M312C+S335R, Y254C+S335R, S250V+S335K, Y254W+S335R, S334G+S335K. S250L+H306R, S250L+S335K, Y254W+S335R, S250V+S335K, S334L+S335K, I309C+S335R, I309C+S335K, Y254F+S335R, S250A+S335R, S250C+S335K, S250I+H306R, S250A+H306R, S250C+S335K, H306Q+S335K, Y254C+S335K, S334L+S335K, S250A+H306R, I309C+S335K, Y254W+S335K, Y254C+S335K, Y254I+S335R, H306R+Y381C, S250M+S335K, M312C+S335K, M312C+S335K, S250V+H306R, S250M+S335R, Y254V+S335R, Y254I+S335K, S250W+S335R, I259C+S335R, S334M+S335R, Y254A+S335R, Y254V+S335R, S250M+S335K. F251L+S35R, Y254I+H306R, I259C+S335K, Y254I+S335R, I259C+S335K, S250Y+S335K, Y254F+S335K, S250M+S335R, Y254F+H306R, Y254V+S335K, Y254V+H306R, S334T+S335K, S334Y+S335R, Y254A+S335R, S334M+S335K, Y254I+H306R, Y254M+S335K, Y254A+S335K, S334F+S335R, S334M+S335K, F251L+H306R, S334T+S335R, Y254I+S335K. Y254A+S335K, S334T+S335K. S250Y+H306R, Y254A+H306R, Y254M+S335K, F251L+S335K, I259C+S335R, F251L+H306R, Y254M+H306R, S334P+S335R, S334P+S335R, S334P+S335K, S334F+S335R, Y254M+S335R, H306I+S335C, H306L+S335C, H306L+S335C, H306I+S335C, H306V+S335C, H306C+S335C, H306I+S334V, H306V+S335C, H306C+S335C, H306I+S334V, H306L+S335V, H306M+S335C, H306I+S335G, H306I+S335G, H306I+S335A, H306I+S335A, H306F+S335C, H306F+S335C, H306L+S335V, H306I+S335T, H306L+S335A, H306C+S335A, H306I+S335V, H306I+S335V, H306L+S334V, H306L+S335A. H306C+S335V, H306L+S334C, H306I+S335I, H306I+S334A, H306L+S334C, H306L+S334V, H306F+S334V, H306C+S335A, H306F+S335V, H306C+S334V, H306C+S334V, H306I+S335L, H306C+S335V, H306L+S334I, H306M+S335C, H306I+S334C, H306I+S335I, H306W+S335C, H306V+S335A, H306I+S334C, H306I+S334I, H306L+S335I, H306V+S335A, H306C+S334C, H306L+S335G, H306C+S334C, H306L+S335T, H306I+S334I, H306V+S334V, H306I+S335L, H306M+S335V, H306V+S335V, H306V+S335V, H306I+S335M, H306L+S335T, H306V+S335I, H306C+S335G, H306L+S335L, H306C+S334I, H306L+S335Y, H306L+S335I. H306V+S334V, H306C+S335G, H306I+S334A. H306L+S334A, H306C+S335I, H306F+S335V, H306I+S335T, H306L+S335Y, H306C+S334I, H306L+S334I, H306V+S335G, H306V+S3350, H306A+S335C, H306A+S335C, H306M+S335A, H306C+S334A, H306V+S335I, H306L+S335G, H306L+S334A, H306C+S334A, H306I+S335Y, H306I+S335Y, H306W+S335C, H306F+S335A, H306F+S335A, H306F+S335I, H306I+S335N, H306L+S335L, H306I+S335M, H306I+S335N, H306V+S334I, H306V+S334C, H306V+S335L, H306C+S335L, H306C+S335L, H3061+A376L, H306F+S335T, H306L+S335M, H306F+S334V, S334V+S335C, S335C+Y381L, S335C+A376I, S334I+S335C, S334V+S335C, M312I+S335C, M312I+S335C, S335C+A376L, S334I+S335C, S335C+A376L, S335C+A378C, S335C+A376C, S335C+A376V, S334A+S335C, S335C+A378C, S334A+S335C, S335C+A3761, S334V+S335A, S335A+A376L, S335C+A376C, S335C+A376V, S335C+A378L, S334V+S335A, S335A+A376I, S334V+A376L, S334I+S335V, S335C+A378I, M312F+S335C, M312F+S335C, S334I+S335A, S335C+A378V, S335V+A376L, S334V+S335L, S335C+A378I, S334C+S335V, S334V+A376I, S334V+A376I, S335V+A376I, M312I+S334V, S334V+S335I, S335C+Y381I, M312V+S335C, S335C+A378L, S335A+Y381L, S334C+A376C, S334V+S335I, S335C+Y381V, M312L+S335C, S335C+A378V, S335V+A376L, S334I+S335V, S335C+Y381C, S335V+A376I, S334C+A376L, S334C+Y381L, M312V+S335C, S334V+S335L, S334C+A376I, S334C+A376C, S334V+Y381L. M312L+S335C, S335V+A376V, S3341+A376I, S335V+A376V, S335V+Y381L, Y254F+S335C, L310F+S335C, S3351+A376I, S334V+A376V, M312I+S335V, S334C+S335A, S334I+A376L, M312F+S335V, S335C+Y381A, L310F+S335C, S334G+S335C, S334G+S335C, S334V+A376V, S335C+A376M, S335C+A376M, S334A+S335I, S334V+A376L, M312C+S335C, S334C+S335A, S335A+A376V, S334A+S335V, S334A+S335V, Y254F+S335C, S335A+A376L, S335A+Y3811, S335A+A376I, S334L+S335C, S335A+A376V, M312C+S335C, D152F, D152L, D152I, D152V, D152M, D152W, D152Y, D152S, D152A, D152T, D152G, D152N, D152Q, D152P, D152H, D152K, D152R, D152C, D155Q, D155L, D155F, D155I, D155M, D155W, D155V, D155Y, D155A, D155N, D155T, D155S, D155P, D155G, D155H, D155R, D155K, D155C, D156L, D156F, D156I, D156W, D156V, D156A, D156Y, D156M, D156G, D156S, D156T, D156Q, D156N, D156P, D156H, D156K, D156R, D156C, H146R, H146K, H153R, H153K, Y63A, H146A, H153A, F74A, L141A, F81A, P72A, K144A, T143A, Y75A, W98A, D156A, R158A, S250W, S250F, S250D, S250E, S250K, S250R, F251A, Y254A, I259W, I259F, I259D, I259E, I259R, I259K, H306A, 1309W, I309F, I309E, I309D, I309R, I309K, L310W, L310F, L310D, L310E, L310R, L310K. M312W, M312F, M312D, M312E, M312R, M312K, S334W, S334F, S334D, S334E, S334R, S334K, S335W, S335F, S335D, S335E, S335K, S335R, M337W, M337F, M337D, M337E, M337K, M337R, A376W, A376F, A376E, A376D, A376R, A376K, F377, A378W, A378F, A378D, A378E, A378K, A378R, F380A, Y381 A, D348I, D348L, D348M, D348F, D348V, D348Y, D348A, D348W, D348T, D348G, D348P, D348H, D348S, D348R, D348K, D348E, D348C, H306R, H306D, H306E, H306K, H307D, H307R, H307E, H307K, H327R, H327K, H327E, H327D, H302E, H302K, H302R, H302D, H215K, H215R, H215E, H215D, H223K, H223R, H223E, H223D, H403K, H403R, H403E, H403D.
   65. Polypeptide, vaccine, method or composition according to any one of embodiments 1 to 64, comprising a polypeptide with at least one P. acnes epitope of the polypeptide according to SEQ ID NO: 49, wherein the polypeptide with at least one P. acnes epitope of the polypeptide according to SEQ ID NO: 49 preferably comprises one or more, preferably one, two or three, especially one, of the following amino acid exchanges compared to SEQ ID NO: 49:
   A274Y, S26C, S26L, A274F, S26I, G119H, A274L, A274W, S26V, S26M, S212L, T108I, A134L, S26F, N130V, A229L, A274I, G194F, S26W, A291F, G181I, A229I, E278I, A175L, G181L, A152L, A274V, N130L, E278V, K46I, E278L, E278A, K46C, K46L, K93L, K93V, E164I, E278C, E164V, E278M, N130I, K46V, R221C, K93C, K46M, S51L, K93I, E278F, K93M, E164A, 551I, R221L, P209L, K46F, S51V, E164C, R221I, E164M, K93F, D137L, P209V, E164L, P59L, R221F, N130C, S51C, R221G, E278T, K46G, P59V, K46A, N130M, E278G, D137V, K46T, P209C, P59I, P209I, T144L, N130F, E164F, S51F, R221M, P209F, S51M, R221S, P209A, K93A, P209M, E164Y, D137C, R221A, P115C, P59F, S208A, K46S, P115V, A1341, P115A, S212V, N249L, E278S, D137F, D137M, P115L, 5212I, R221V, N249V, D137I, E278Y, G194L, P59M, R221T, A188L, N249I, T144I, E164T, S208C, S208V, D137A, K46Y, K93G, 087I, E164G, K46P, P59C, A97V, S212C, G83L, K93T, N249C, A134V, G83V, T108V, T112L, T144V, A100L, G87V, E164W, E164S, A171L, A97L, A188F, K93W, T901, A174I, K93Y, A111L, A971, N130A, G48I, E278W, R221Y, G135L, T112V, F197V, T144F, K93S, G194V, G87A, A188V, T144C, K46N, P115M, A1751, N130T, S51A, G194C, G48L, G104V, A174V, G158V, T90V, R221N, A202L, A39L, A134F, D137Y, 0158I, A174L, P155V, A171V, S51T, F197I, T112C, D137W, G135V, D137G, P115G, T250C, S208L, A100I, T250L, T144M, T108C, P115F, G69L, T112F, G181F, A100F, S208I, G223V, T250V, N249M, A111V, N249Y, A111F, A188M, A111I, P209Y, A175V, P1551, N130G, S120V, A111C, A100C, A97F, G194M, 069I, E278Q, S208G, 0181C, 087F, G48V, A97C, I205L, G194I, D137T, S51G, F197C, G223L, N249A, S51Y, P115S, A202I, P155C, S26A, G83M, S120C, D137S, E278N, G83F, G158C, P59Y, 087C, G87M, N130S, P115I, A202F, A100M, G83C, S212M, A39V, A188I, F197L, G181M, A134C, P115T, A174F, K93P, P209W, P209G, P59A, A39C, G104I, T250G, S208F, R221Q, G181A, G104C, T90C, K46Q, P209T, K46W, A171I, G158A, A134M, G48C, G194A, T90A, T90M, N130Y, N249F, A172L, A152V, A1521, P59W, R221P, G158F, T112M, 0135M, G181V, G104L. S208T, N249W, A97M, G135F, K93Q, G42L, A111M, G104A, E278P, T90L, R221D, A175C, A188C, A291L, S212A, F123L, T250I, A174C, E164Q, P155L, A174M, G158M, A172V, E164N, A168L, T90F, A39F, G42V, I205F, A39M, N249G, A175F, G135C, A171F, S212F, K46D, A100Y, G421, P59G, G69A, E164P, G158L, G42C, A171C, T250M, T108L, K93N, A171M, G223I, A175M, T108M, A1721, S51P, A291I, A111Y, G20V, S1201, P59T, A58I, G223C, S208M, G48M, K46H, A62L, A43I, T250A, N130W, G194Y, R221H, D137P, N249S, G104F, A100V, F123I, A172C, T112I, G69F, P115Y, P59S, A100W, G104M, G69C, R221W, K93H, A204L, G20L, G223F, A168I, D28K, E275K, K77R, D17R, H76D, H75D, H76E, K77D, H75E, P294D, S293D, S293E, D286E, P294E, S292E, D285E, H76R, K77E, K289R, P295D, D285K, R179E, H75R, P295E, P294R, P294K, D286K, D285R, S293R, S293K, R180D, R180K, H183E, R179K, K289E, R180E, A98K, R179D, A99K, S292D, D286R, K290D, H75K, A99E, N256E, K290E, K290R, R239D, A199K, A200K, K255E, H183D, A99R, H235E, A98R, D102K, and A200R.
   66. Vaccine, composition, method or DsA1 and/or DsA2 derivative according to any one of embodiments 1 to 63 wherein the DsA1 domains are defined as follows: NSR: from S29 to I48, CSD1 from I49 to L130, SR1 from G131 to S147, CSD2 from A148 to L267, SR2 from A268 to T277, CSD3 from A278 to K323, the PT repeat region from P324 to T361, and CTR from S362 to F405 in the sequence of Q6A5X9 or any other functional DsA1 sequence comprising all these domains, especially the DsA1 sequences of FIG. 12 comprising all these domains;
   and/or wherein the DsA2 domains are defined as follows: NSR: from A72 to K92, CSD1 from 193 to L174, SR1 from S175 to S191, CSD2 from A192 to L311, SR2 from A312 to T321, CSD3 from A322 to E366, the PT repeat region from P367 to T420, and CTR from H421 to A463 in the sequence of Q6A5P9 or any other functional DsA2 sequence comprising all these domains, especially the DsA2 sequences of FIG. 12 comprising all these domains;
   and/or wherein the PITP domains are defined as follows: ENFD from A32 to R164; SR1 from E165 to K237, HbD from V238 to L396, SR2 (including C-terminal LPXT(G) motif (i.e. including LPXT, but not the G)) from S397 to T430, and hLAR from G431 to 1467 in the sequence of Q6A9N1 or any other functional PITP sequence comprising all these domains, especially the PITP sequences of FIG. 12 comprising all these domains.
   67. Pharmaceutical composition according to any one of claims 1 to 64 comprising in a liquid formulation or in a dry or frozen formulation which is reconstitutable to a liquid formulation: 0.01 mg/ml to 50 mg/ml, preferably 0.05 to 5 mg/ml, especially 0.1 to 1 mg/ml, polypeptide as defined above comprising at least one P. acnes epitope. 0.1 to 100 mM, preferably 0.5 to 50 mM, especially 0.1 to 10 mM, NaH₂PO₄, 0.1 to 100 mM, preferably 0.5 to 50 mM, especially 0.1 to 10 mM, Na₂HPO₄, 0.1 to 100 mM, preferably 0.5 to 50 mM, especially 0.1 to 10 mM, KH₂PO₄, 1 to 1000 mM, preferably 5 to 500 mM, especially 50 to 300 mM. NaCl, 0.1 to 100 mM, preferably 0.5 to 50 mM, especially 0.1 to 10 mM, KCl, and/or 0.01 to 1%, preferably 0.05 to 0.5%, especially 0.1 to 0.5%, aluminium adjuvant (aluminium hydroxide).
   68. Pharmaceutical composition according to any one of claims 1 to 67 having in a liquid formulation or in a dry or frozen formulation which is reconstitutable to a liquid formulation a pH of 4.5 to 7.4, preferably of 4.7 to 7.3, even more preferred of 5.0 to 7.3, especially of 5.3 to 7.2.
   69. Pharmaceutical composition comprising a P. acnes polypeptide or vaccine according to any one of embodiments 1 to 68, eliciting a cross-type reactivity at least against Types IA1, IA2, and II of P. acnes and/or eliciting a cross-type reactivity at least against Types IAI1, IA2, IB, II and III of P. acnes.
   70. Pharmaceutical composition comprising a P. acnes polypeptide or vaccine according to any one of embodiments 1 to 69, wherein the polypeptide is present in the composition at least partially in a form wherein it lacks an N-terminal methionine residue.
   71. A polypeptide comprising at least one polypeptide stretch of Dermatan sulfate-binding adhesin 1 of P. acnes (DsA1) and at least one polypeptide stretch of Dermatan sulfate-binding adhesin 2 of P. acnes (DsA2), said DsA1 and DsA2 comprising from N- to C-terminus an N-terminal region, a first conserved sub-domain (“CSD1”), a first swapping region (“SR1”), a second conserved sub-domain (“CSD2”), a second swapping region (“SR2”), a third conserved sub-domain (“CSD3”); and, optionally a Pro-Leu repeat containing region (“PT repeat region”), and a C-terminal region;
   wherein the polypeptide comprises at least CSD1, CSD2 or CSD3 of DsA1 and at least CSD1, CSD2 or CSD3 of DsA2.
   72. A polypeptide comprising at least one polypeptide stretch of Dermatan sulfate-binding adhesin 1 of P. acnes (DsA1) and at least one polypeptide stretch of Dermatan sulfate-binding adhesin 2 of P. acnes (DsA2), said DsA1 and DsA2 comprising from N- to C-terminus an N-terminal region, a first conserved sub-domain (“CSD1”), a first swapping region (“SR1”), a second conserved sub-domain (“CSD2”), a second swapping region (“SR2”), a third conserved sub-domain (“CSD3”); and, optionally, a Pro-Leu repeat containing region (“PT repeat region”), and a C-terminal region;
   wherein the polypeptide stretch of DsA1 and DsA2 has independently a length of at least 20 amino acid residues.
   73. A polypeptide according to embodiment 71 or 72, wherein at least either the polypeptide stretch of DsA1 or DsA2 comprises or consist of a DsA1 or DsA2 epitope; or wherein the at least two polypeptide stretches form a DsA1 or DsA2 epitope.
   74. A polypeptide according to any one of embodiments 71 to 73, comprising at least two of CSD1, CSD2 and CSD3 of DsA1 or DsA2.
   75. A polypeptide according to any one of embodiments 71 to 74, having at least two polypeptide stretches of DsA1 or DsA2, wherein CSD1, CSD2 and/or CSD3 of DsA1 or DsA2 are connected by SR1 and/or SR2, wherein SR1 and SR2 are of DsA1 or DsA2 or a hybrid SR, wherein the hybrid SR consist of amino acid residues being present either in a DsA1 or in a DsA2 SR.
   76. A polypeptide according to any one of embodiments 71 to 75, wherein the polypeptide stretch comprises or consists at least of a CSD2 fragment, wherein the CSD2 fragment is preferably
a contiguous polypeptide sequence of phenylalanine 150 (F150) to leucine 184 (L184),
a contiguous polypeptide sequence of phenylalanine 150 (F150) to leucine 267 (L267), or
a contiguous polypeptide sequence of histidine 218 (H218) to leucine 267 (L267),
   and wherein the amino acid numbering corresponds to the amino acid sequence Q6A5X9 in the UniProt database.
   77. A polypeptide according to any one of embodiments 71 to 76, wherein the polypeptide stretch comprises or consists at least of
a contiguous polypeptide sequence of phenylalanine 150 (F150) to isoleucine 193 (I193).
a contiguous polypeptide sequence of phenylalanine 150 (F150) to leucine 267 (L267), or
a contiguous polypeptide sequence of histidine 218 (H218) to leucine 267 (L267).
   78. A polypeptide according to any one of embodiments 71 to 77, wherein the polypeptide stretch is
   (1) a contiguous polypeptide sequence of phenylalanine 150 (F150) to leucine 184 (L184), further extending one, two, three, or four amino acids at the N-terminus and/or one, two, three, four, five, six, seven, eight, nine, or ten amino acids at the C-terminus;
   (2) a contiguous polypeptide sequence of phenylalanine 150 (F150) to leucine 267 (L267), further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus; and/or
   (3) a contiguous polypeptide sequence of histidine 218 (H218) to leucine 267 (L267) further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus.
   79. A polypeptide according to any one of embodiments 71 to 78, wherein the polypeptide stretch has a length of at least 35 amino acids, preferably a length of at least 40 amino acids, especially a length of at least 50 amino acids.
   80. A polypeptide according to any one of embodiments 71 to 79, which comprises at least one additional polypeptide with an amino acid sequence containing or consisting of at least one additional P. acnes antigen or P. acnes epitope which is not a DsA1 or DsA2 antigen or epitope, preferably at least one additional amino acid sequence containing at least the putative iron-transport protein (PITP) polypeptide of P. acnes or at least one epitope of PITP polypeptide of P. acnes, wherein the PITP polypeptide comprises from N- to C-terminus an extended neocarzinostatin family domain (“ENFD”), a first swapping region (“SR1”), a heme-binding domain (“HbD”), a second swapping region (“SR2”) including the C-terminal LPXTG motif, and a hydrophobic C-terminal region (“hLAR”).
   81. A polypeptide according to embodiment 80 wherein the additional polypeptide is the PITP polypeptide or a fragment or derivative thereof, wherein the fragment or derivative thereof comprises a sequence comprising at least one of ENFD, SR1, HbD, SR2, preferably selected from ENFD and HbD or an epitope containing fragment thereof.
   82. A polypeptide according to any one of embodiments 71 to 81, wherein the at least one polypeptide stretch comprises a fragment of DsA1 or DsA2 being
   (1) a contiguous polypeptide sequence of phenylalanine 150 (F150) to leucine 184 (L184), optionally further extending one, two, three, or four amino acids at the N-terminus and/or one, two, three, four, five, six, seven, eight, nine, or ten amino acids at the C-terminus;
   (2) a contiguous polypeptide sequence of phenylalanine 150 (F150) to leucine 267 (L267), optionally further extending one, two, three, or four amino acids at the N-terminus and/or at least ten, eleven, twelve, thirteen, fourteen, fifteen, sixteen, seventeen, eighteen, nineteen, twenty, twenty-one, or twenty-two amino acids at the C-terminus;
   (3) a contiguous polypeptide sequence of histidine 218 (H218) to leucine 267 (L267) optionally further extending at least ten, eleven, twelve, thirteen, fourteen, fifteen, sixteen, seventeen, eighteen, nineteen, twenty, twenty-one, or twenty-two amino acids at the N-terminus and/or one, two, three, four, five, six, seven, eight, nine, or ten amino acids at the C-terminus.
   83. Vaccine comprising a polypeptide according to any one of embodiments 71 to 82, and, optionally, a pharmaceutically acceptable excipient.
   84. A polypeptide according to any one of embodiments 71 to 82 or a vaccine according to embodiment 83 for use in a therapeutic treatment, preferably for use in the treatment or prevention of P. acnes-associated infections, especially selected from the group consisting of acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes.
   85. A polypeptide according to any one of embodiments 71 to 82 or a vaccine according to embodiment 83 for use in the treatment or prevention of P. acnes-associated infections in a human patient suffering from P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes.
   86. Pharmaceutical formulation, especially vaccine, according to any one of embodiments 1 to 85, comprising a PITP polypeptide and/or a porphyrin-binding PITP fragment and/or a porphyrin-binding PITP derivative, further comprising a porphyin molecule and/or a porphyrinogen molecule and/or a porphyrinogen or porphyrin degradation product bound to the PITP and/or a porphyrin-binding PITP fragment and/or a porphyrin-binding PITP derivative, wherein the porphyin molecule and/or porphyrinogen molecule and/or porphyrinogen or porphyrin degradation product preferably contains a metal ion in complex form, wherein the metal ion is preferably a Fe, Co, Zn, Mg, Se, or Cu ion, especially a Fe²⁺ or a Fe³⁺ ion.
   87. Pharmaceutical formulation, especially vaccine, according to any one of embodiments 1 to 86, comprising a PITP polypeptide and/or a porphyrin-binding PITP fragment and/or a porphyrin-binding PITP derivative, further comprising a porphyin molecule and/or a porphyrinogen molecule and/or a porphyrinogen or porphyrin degradation product bound to the PITP and/or a porphyrin-binding PITP fragment and/or a porphyrin-binding PITP derivative, wherein the porphyin molecule and/or porphyrinogen molecule and/or porphyrinogen or porphyrin degradation product is selected from the group hemin, protoprophyrin IX, coproporphyrin I, II and/or III, especially coproporphyrin III; pentacarboxyporphyrin, uroporphyrin, heptacarboxyporphyrin, hexacarboxyporphyrin, especially as form I or III; nitro-porphyrins, especially 5,10,15,20-tetraphenylporphyrin (TPP), 5,10,15,20-tetra(4′-fluorophenyl)porphyrin (TpFPP), 5,10,15,20-tetra(4′-chlorophenyl)porphyrin (TpClPP), 5,10,15,20-tetra(4′-bromophenyl)porphyrin (TpBrPP), biliverdin, 5,10-diphenyl-15,20-di(N-methylpyridinium-4-yl)porphyrin (Di-cis-Py+), PORF-1 to PORF-34, porphine, octaethylporphyrin, tetraphenylporphyrin, verteportin, ascoproporphyrin, hydroxymethylbilane (HMB), mesoporphyrin IX, 7-carboxyporphyrin (7P), 6-carboxylporphyrin (6P), heme A, heme B, heme C, heme O, heme I, heme m, heme D, S heme; uroporphyrinogen III, chlorophylls, especially chlorophyll a, chlorophyll b, and bacteriochlorophylls.
   88. Vaccine or pharmaceutical formulation as defined in any one of embodiments 1 to 87 for use in the treatment or prevention of P. acnes-associated infections, comprising a polypeptide comprising an epitope of DsA1 and/or DsA2 and/or PITP, wherein the epitope is selected from the group consisting of R32-I41, Q38-K51, R32-K51, T43-K51, Q38-K51, R87-K90+T43-K51, R87-K90+T117-I132 and R87-K90+S234-G250, R87-K90+L246-A260, R87-K90+A256-E270, R87-K90+R266-T277, T117-I132, T117-A127, V128-I132, A144-N157, H146-A160, A148-N157, A156-A170, K166-L180. A176-T190, P186-A198, N181-E191, I216-F224, I216-D225, A226-A240, S234-G250, I251-I263, I264-P271, P236-G250, L246-A260, A256-E27G, S234-G250, I251-I263, I251-L267, A268-L280, R266-T277, T285-R286+I216-F224, T285-R286+I264-P271, T285-R286+V289-K296, T285-R286+V289-K296, T285-R286+A144-N157, A310-D313+T285-R286, T285-D290, T285-D290+V291-T300, T285-D290+A301-E307, V291-T300, A301-E307, T285-T300, A301-E307, R286-D290+V291-T300, R286-D290+A301-E307, R286-T300, V289-K296, A310-D313+I216-F224, A310-D313+I264-P271, A310-D313+T285-R286, A310-D313+R286-D290, A310-D313+V289-K296, A310-D313+V289-K296, A310-D313+T285-T300, A310-D313+A144-N157. A310-D313+T285-R286, A310-D313+T285-D290, A310-D313+T293-E307, T285-D290, V291-T300, T293-E307, A301-E307 of DsA1; L152-Q166, G190-P230, I199-D208, A218-I237, P230-Q244, I231-A270, H254-A270, A271-S279, A271-R310, L311-T321, L311-K323, V333-Q347, A218-P230, 1231-I237, H254-H262, Q256-H262, E261-D269. D269-S279, K313-K323, of DsA2; D79-T90, E73-D85, R43-I50, P68-Y75, P86-E92, 139-G45, Y84-D89, F81-D89, D79-T90, T37-E44, E73-W98, E73-F81, D89-T90, P72-F81, A129-F138, D120-Q134, F111-D120, F132-G147. D152-E165, R115-F123. D120-K128, P131-F138, N181-E191, T143-T159, P116-T124, P131-D137, P131-D137, T175-C231, Q198-K203, P179-K185, G200-Q210, K174-A188, K174-K185, P201-Q209, P183-P201, P183-K191, K185-P195, R164-S180, E165-S180, K185-S190, V193-N202, V193-G200, K203-P208, R216-T225, R216-R224, P173-K191, K197-K203, P168-T175, K185-K203, R164-K174, T175-V193, S250-N261, D287-S300, K340-V347, D338-F352. D338-D348, S285-P288+G305-L314, S285-P288+H306-L314, S285-P288+T342-T351, S285-P288+D338-D348, D287-S300, T342-T351, D338-D348, H306-L314, G305-L314, G364-K375, R382-E399, V367-G373, A383-L390, T342-T351, M387-T395, E385-T392, V401-V410, N404-A409, G416-L427, L396-V410, T406-I415, D417-G424, V407-D418, V407-V414, K421-V429, S419-T430, D408-I415, T406-V414, of PITP, preferably wherein the epitope comprises at least one additional amino acid residue at the N- or C-terminus of the DsA1, DsA2, or PITP sequence, especially wherein the epitope comprises at least two additional amino acid residue at the N- or C-terminus of the DsA1, DsA2, or PITP sequence; wherein the polypeptide is preferably covalently linked to a carrier molecule or embedded in a scaffold molecule, especially a carrier polypeptide.
   89. Vaccine or pharmaceutical formulation as defined in any one of embodiments 1 to 87 for use in the treatment or prevention of P. acnes-associated infections, comprising a polypeptide comprising an epitope of DsA1 and/or DsA2 and/or PITP, wherein the polypeptide is a DsA1/DsA2/PITP derivative with the motif K[NYI] starting at position 5 or 6 of the polypeptide and/or having on DNA level the sequence AADUAU or AAVAUU at this coding position.
   90. Vaccine or polypeptide according to any one of embodiments 1 to 89, wherein the DsA1 and/or DsA2 and/or PITP, and/or a fragment or derivative of DsA1 or DsA2 or PITP is provided as DNA or RNA encoding an antigen or an epitope containing fragment or derivative thereof, preferably as mRNA vaccine, especially an mRNA vaccine formulated with cationic polymers, with an mRNA molecule with the following structure: 5′UTR-signal peptide-encoded antigen or epitope-3′UTR: or as a vector-based vaccine, especially with an adenovirus vector, an adeno-associated virus vector, a vesicular stomatitis virus vector, a Newcastle Disease virus vector, an alphavirus vector, a baculovirus vector, or a retrovirus vector; and/or wherein the coding sequence on DNA level has the initial coding DNA sequence ATGGTG: especially wherein the mRNA comprises a sequence as transcribed from SEQ ID NOs: 67 to 76.
   91. Pharmaceutical formulation comprising a vaccine or polypeptide according to any one of embodiments 1 to 90 comprising the polypeptide as defined in any one of these claims, i.e. a DsA1 and/or DsA2 and/or PITP, and/or a fragment and/or a derivative of DsA1 and/or DsA2 and/or PITP according to any one of embodiments 1 to 90 or mixtures thereof; and a pharmaceutically acceptable excipient or carrier.
   92. Method of treatment or prevention of P. acnes-associated infections in a human patient comprising administration of an effective amount of a DsA1 and/or DsA2 and/or PITP, and/or a fragment and/or a derivative of DsA1 and/or DsA2 and/or PITP according to any one of embodiments 1 to 91 or mixtures thereof in an effective amount to a patient in need thereof.
   93. Method of treatment or prevention of P. acnes-associated infections in a human patient suffering from P. acnes-associated infections and pathological conditions associated with any of Type I, II, or III P. acnes, or a combination of at least two phylotypes of Type I, II and III, or of at least two ribotypes of P. acnes, preferably for use as a cross-reactive vaccine, especially a cross-type-reactive vaccine, against P. acnes, especially for the treatment or prevention of infections in a human patient suffering from P. acnes-associated infections and pathological conditions associated with Type IB, and III of P. acnes comprising administration of an effective amount of a DsA1 and/or DsA2 and/or PITP, and/or a fragment and/or a derivative of DsA1 and/or DsA2 and/or PITP according to any one of embodiments 1 to 91 or mixtures thereof in an effective amount to a patient in need thereof.
   94. Method according to claim 93, wherein administration is performed by intradermal, subcutaneous (s.c.), parenteral, intramuscular (i.m.), mucosal, transcutaneous or topical administration, preferably by intradermal, or intramuscular administration, especially by a syringe or by microneedeling devices.
   95. Method of production of the polypeptides, vaccines and formulations according to any one of embodiments 1 to 91, wherein the polypeptides comprising at least one P. acnes epitope as defined in these claims are expressed in a host cell, extracted and purified from these host cells, and, optionally, formulated and finished to a pharmaceutical formulation, especially a vaccine for use in the treatment or prevention of P. acnes-associated infections in a human patient.
   96. Vaccine according to any one of embodiments 1 to 91 comprising a polypeptide
   (a) comprising at least an epitope-containing fragment or derivative of CSD1 of DsA1 and at least an epitope-containing fragment or derivative of CSD2 of DsA2; and/or
   (b) comprising at least a polypeptide stretch comprising a CSD1 fragment or derivative of CSD1 of DsA1 of at least 30 amino acid residues, preferably at least 40 amino acid residues, and at least a polypeptide stretch comprising a CSD2 fragment or derivative of CSD2 of DsA1 of at least 30 amino acid residues, preferably at least 40 amino acid residues;
   (c) comprising at least
a contiguous polypeptide sequence of phenylalanine 150 (F150) to leucine 184 (L184),
a contiguous polypeptide sequence of phenylalanine 150 (F150) to leucine 267 (L267), or
a contiguous polypeptide sequence of histidine 218 (H218) to leucine 267 (L267) of DsA1, and
a contiguous polypeptide sequence of phenylalanine 194 (F194) to leucine 228 (L228),
a contiguous polypeptide sequence of phenylalanine 194 (F194) to leucine 311 (L311), or
a contiguous polypeptide sequence of histidine 262 (H262) to leucine 311 (L311) of DsA2,
   especially wherein the polypeptide comprises an amino acid exchange at one or more of C53. C319 and C321 of DsA1, and C97 and C363 of DsA2, if present in the polypeptide, preferably one or more of amino acid exchanges C53S, C319S and C321P of DsA1 and C97S and C363S of DsA2 and/or wherein in the fragment or derivative of DsA1 or DsA2 at least 5 PT repeats, preferably at least 10 PT repeats, especially at least 15 PT repeats, are deleted compared to a naturally occurring wild type DsA1/DsA2 polypeptide (“native DsA1/DsA2”), and wherein preferably at least one, more preferred at least two, more preferred at least three, even more preferred at least four, especially five, PT repeat(s) is/are present.
   97. Vaccine according to any one of claims 1 to 96, wherein the polypeptide further comprises a PITP polypeptide or a fragment or derivative of PITP comprising a polypeptide stretch of at least 30 amino acid residues comprising at least an epitope of PITP, preferably wherein the PITP polypeptide or a fragment or derivative of PITP comprises at least
a contiguous polypeptide sequence of proline 34 to glutamic acid 73 of ENFD or a PITP derivative thereof,
a contiguous polypeptide sequence of proline 94 to threonine 143 of ENFD or a PITP derivative thereof, or
a contiguous polypeptide sequence of valine 238 to asparagine 393 of HbD or a PITP derivative thereof;
   especially wherein the PITP derivative comprises an amino acid exchange exchange at positions C231 and C402.
   98. Method of production of the polypeptides, vaccines and formulations according to any one of embodiments 1 to 91, wherein the polypeptides comprising at least one P. acnes epitope as defined in these claims are expressed or produced as an mRNA in a host cell, extracted and purified from these host cells, and, optionally, formulated and finished to a pharmaceutical formulation, especially a vaccine for use in the treatment or prevention of P. acnes-associated infections in a human patient.
   99. Method according to embodiment 98, wherein the host cell is an eukaryotic or prokaryotic host cell, preferably a prokaryotic host cell, especially E. coli or L. lactis; or a host cell which is able to glycosylate the expressed polypeptides as defined in any one of embodiments 1 to 96.
   100. Method according to embodiment 98 or 99, wherein the host cell is naturally unable to synthesize porphyrins and wherein preferably porphyrins are added to the culture, to the lysis buffer, and/or to the purified protein.
   101. Method according to any one of embodiments 98 to 100, wherein the polypeptides of SEQ ID NOs:1 to 66 or encoded by SEQ ID NOs:67 to 76 are expressed in the host cells.

### 6 SHORT DESCRIPTION OF THE FIGURES

The invention is further described by the following examples and the figures, yet without being limited thereto.

FIG. 1A

Binding of murine serum antibodies generated by single antigen immunizations to the cell surface of P. acnes strains from the representative genetic types. Murine pre-immune and immune sera (n=5) raised against Alhydrogel® formulations of P022-V2 (DsA1), P027-V3 (DsA2), P028 (PITP) and other P. acnes surface antigens (P002, P005, P018, P032, P035, P042, P046, P068, P069, P070 and P071) were analysed in a flow cytometry-based bacterial surface binding assay. Bacteria were cultivated in the presence of the iron-chelator deferoxamine. Cultures of genetic types IA1 (NCTC737), IA2 (P.acn31), IB (KPA171202), IC (PV66), II (HL050PA2) and III (Asn12) were re-suspended in HBSS buffer containing 2% BSA and incubated with 1:1000 diluted immune sera. Bound antibodies were detected with an Alexa Fluor 488 conjugated goat anti-mouse IgG F(ab′)2 fragment. Median fluorescence intensity (MFI) values were measured on a BD Accuri C6 plus instrument, after cross-linking with 2% PFA. The MFI values of the samples tested in different experiments were compared based on the normalization in reference to the internal assay control.

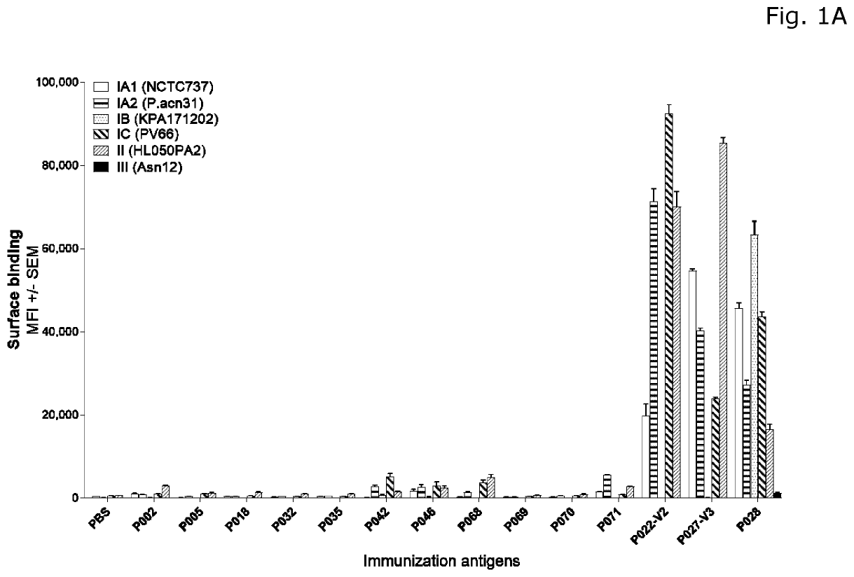

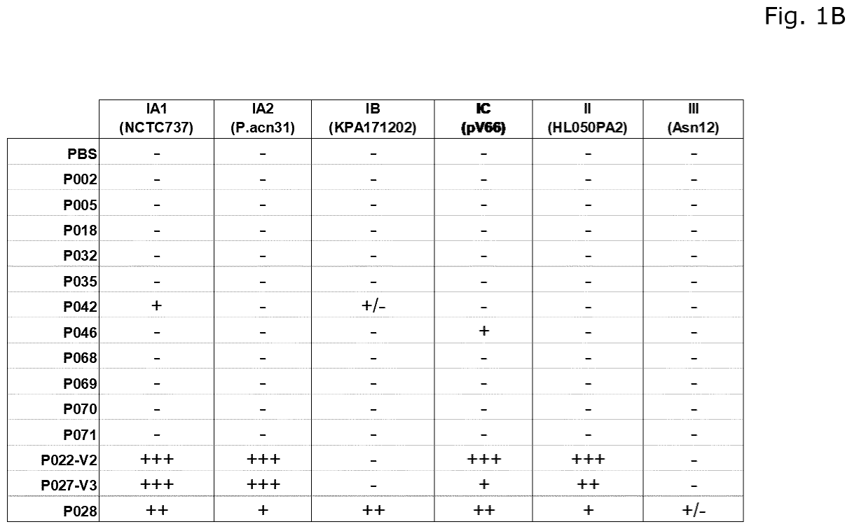

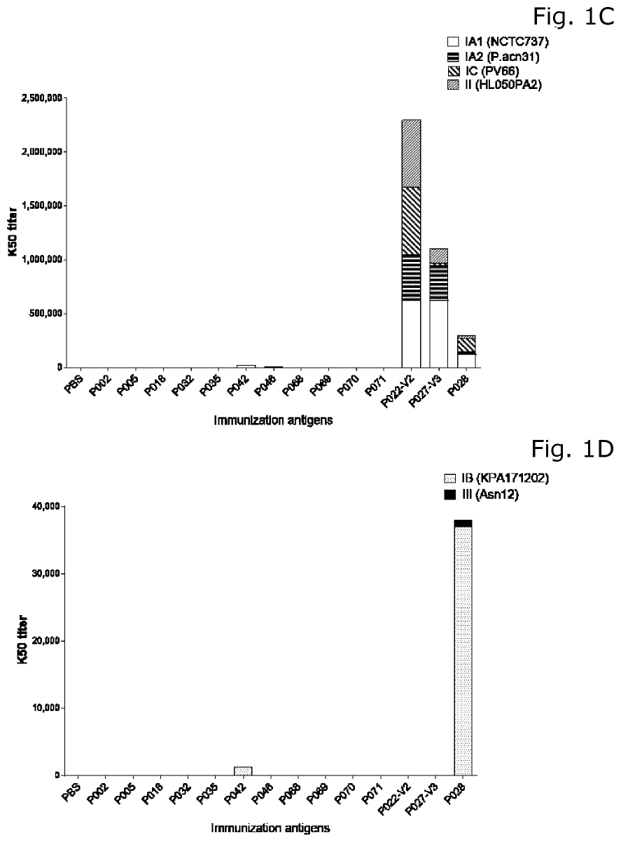

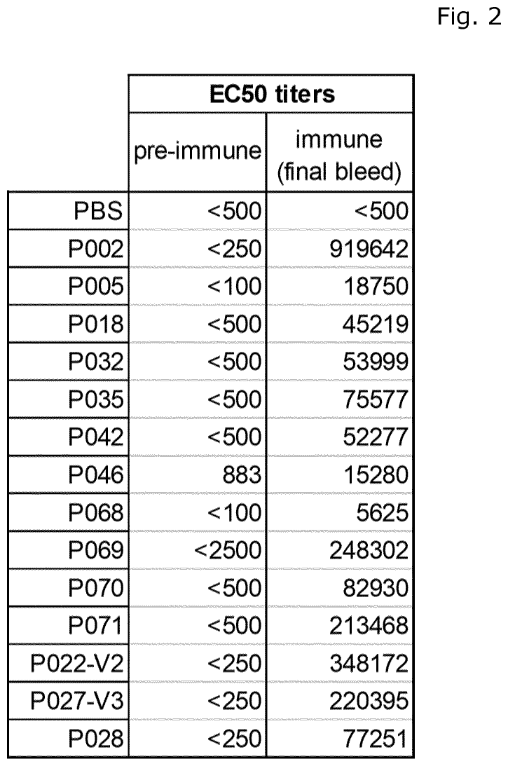

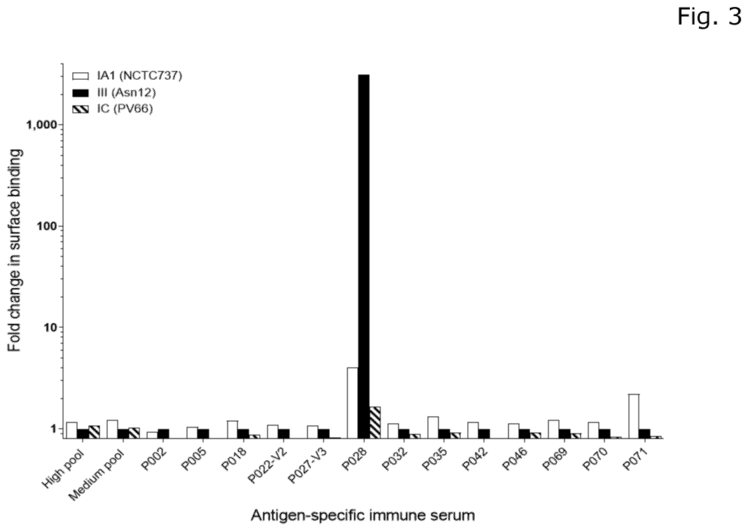

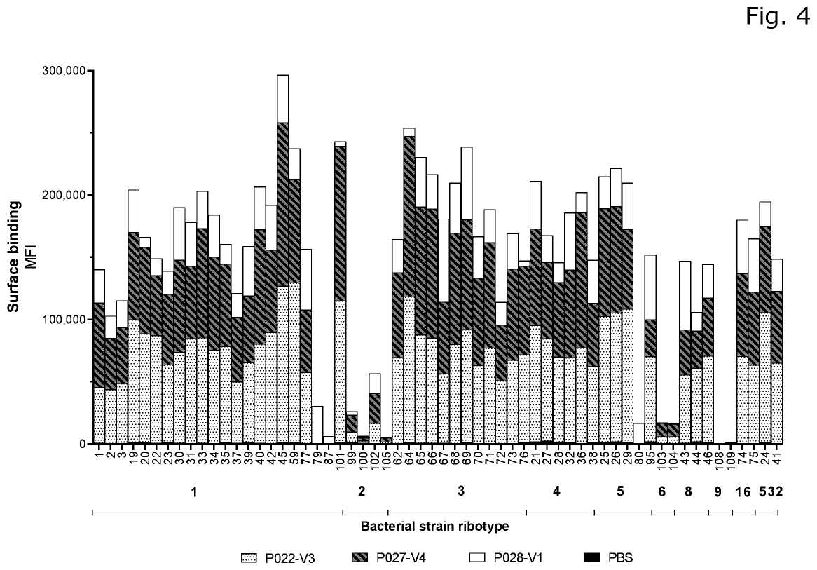

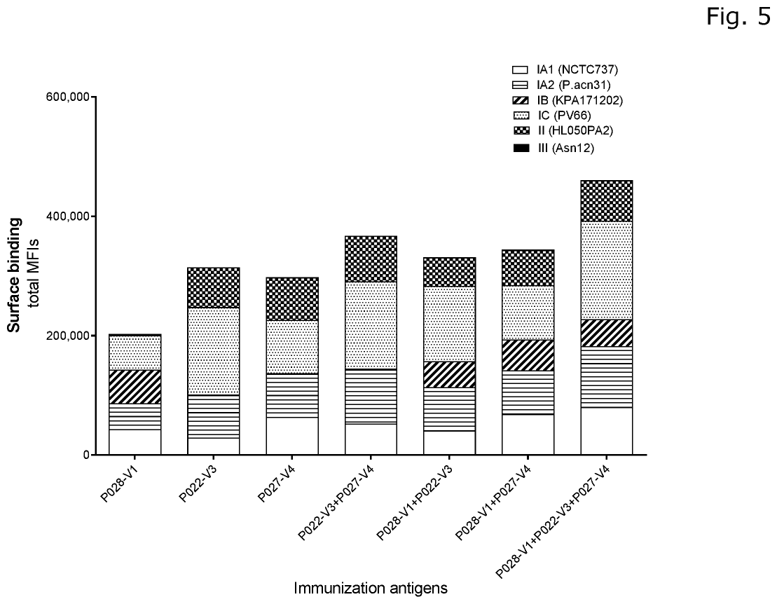

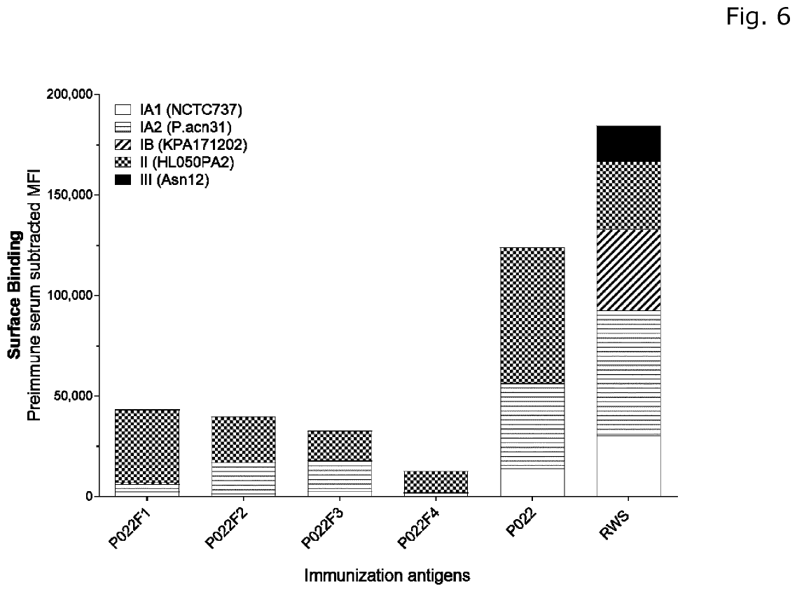

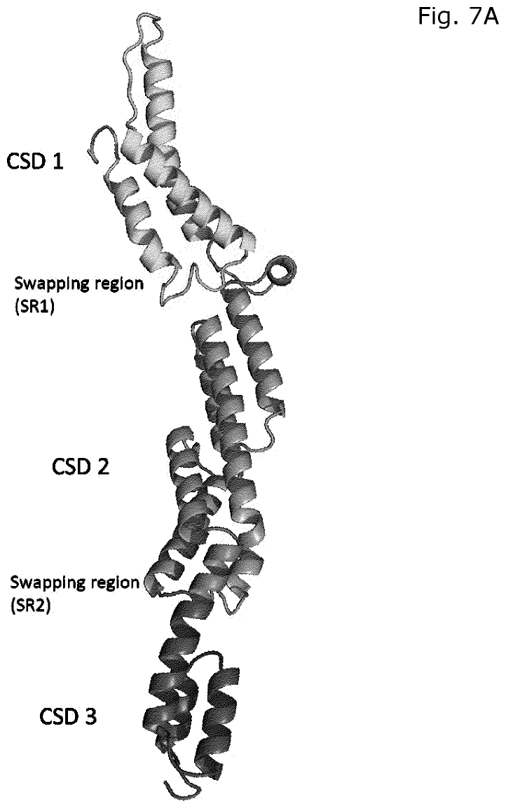

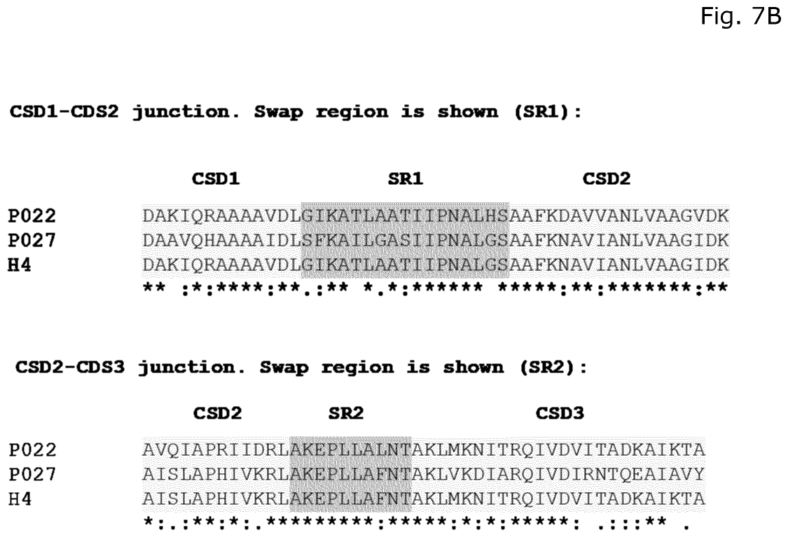

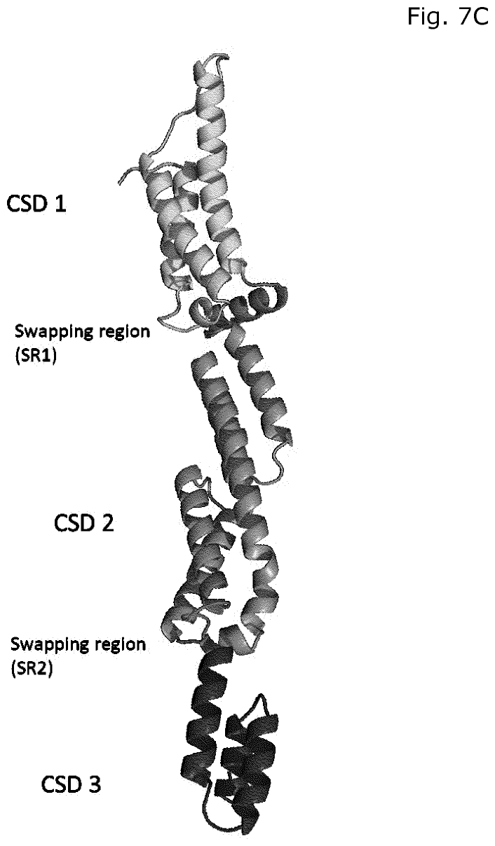

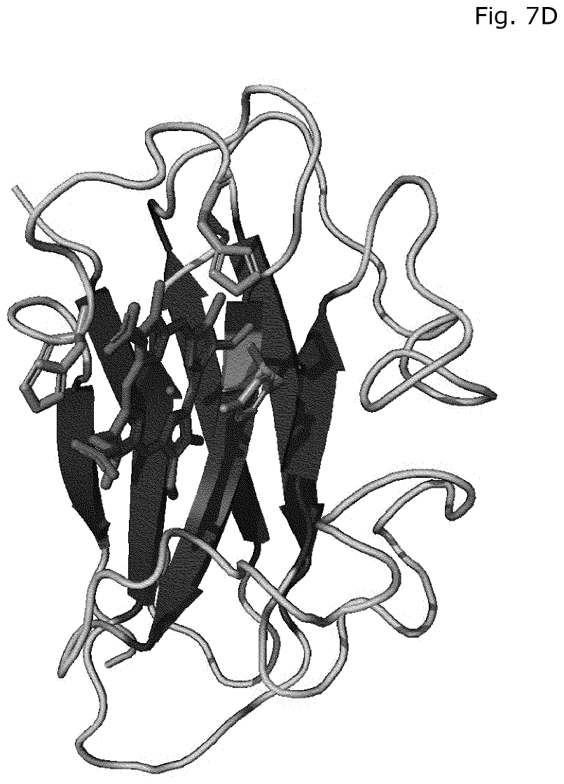

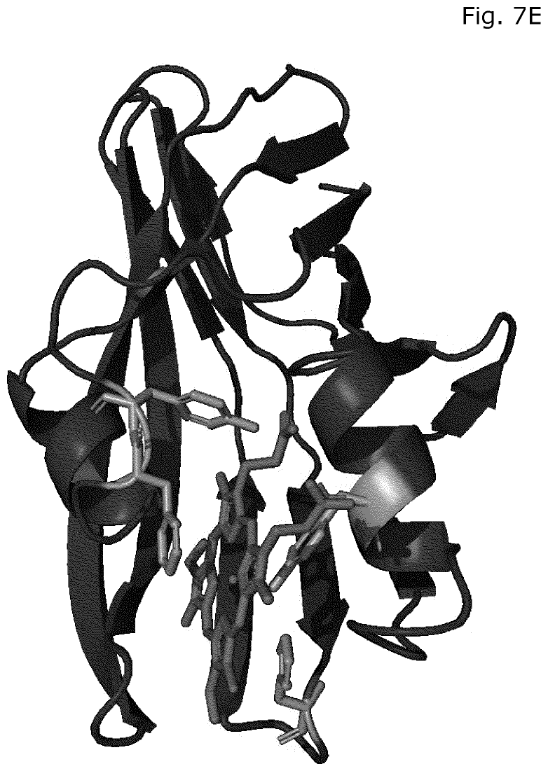

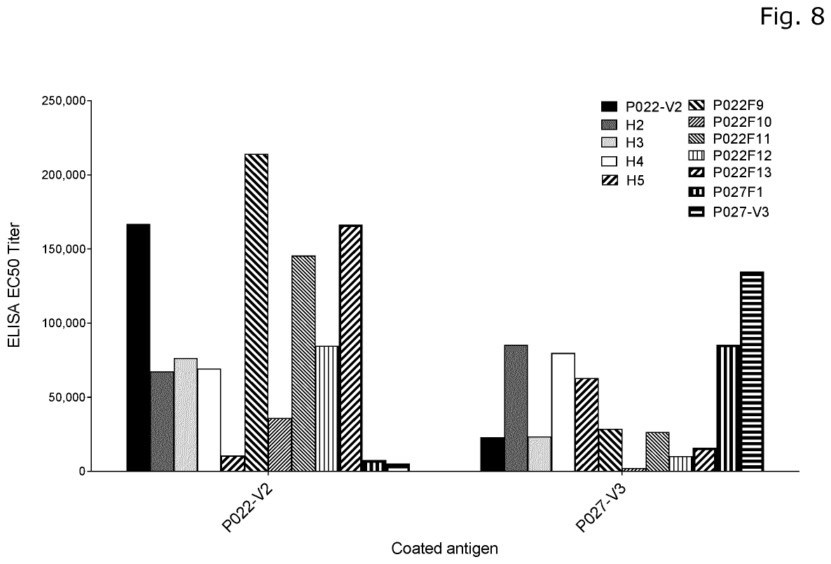

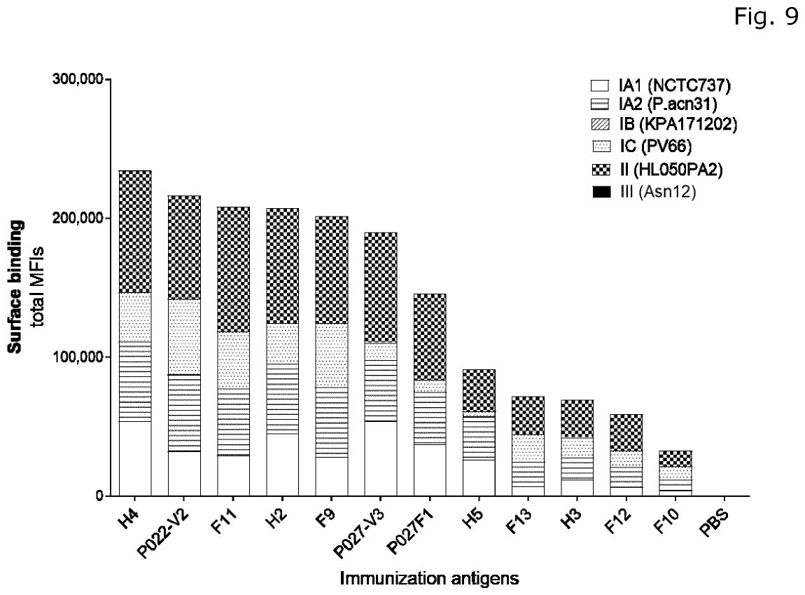

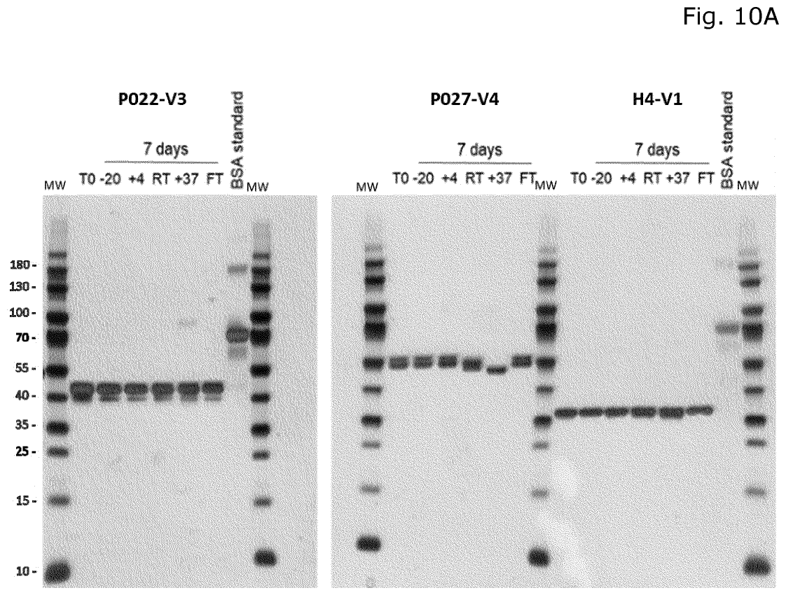

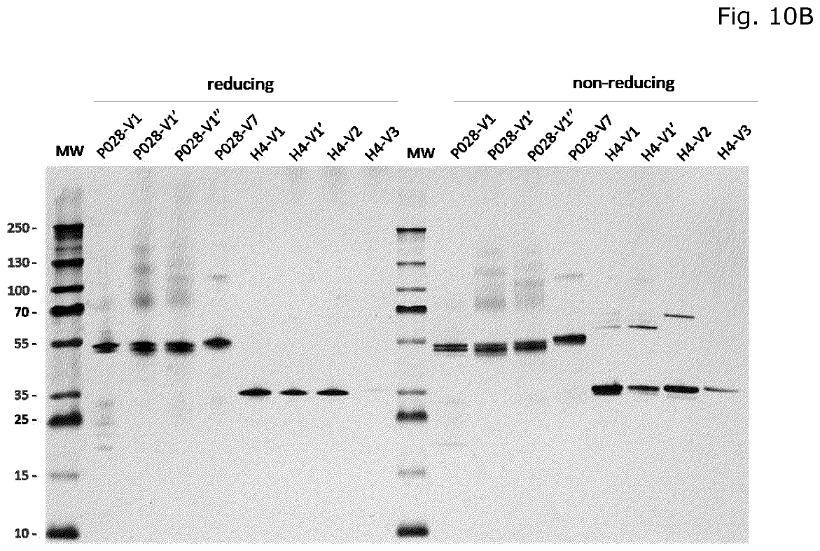

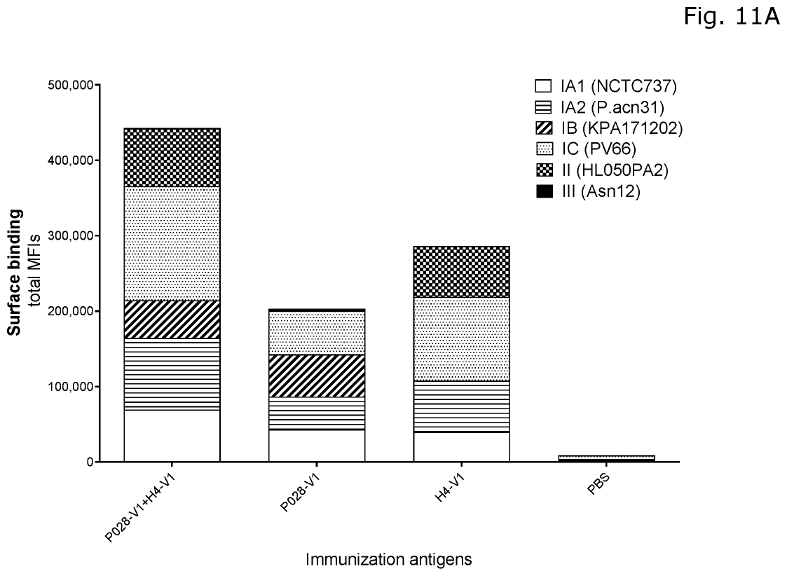

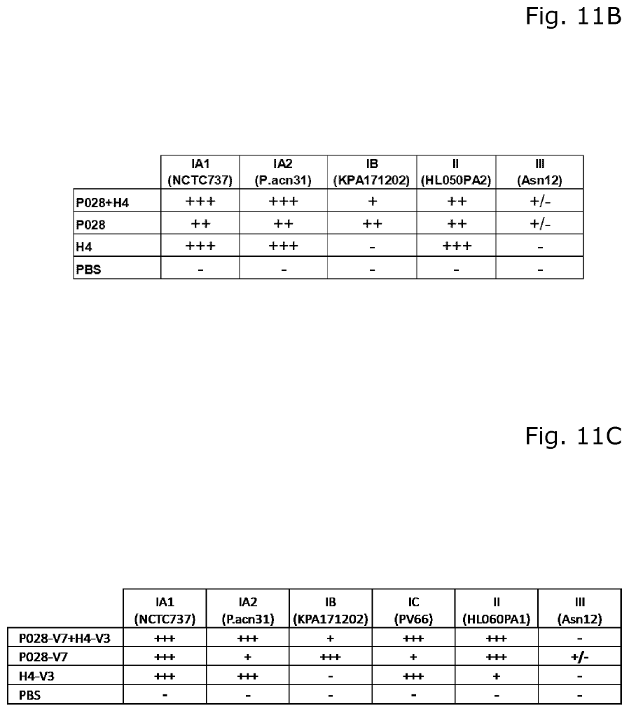

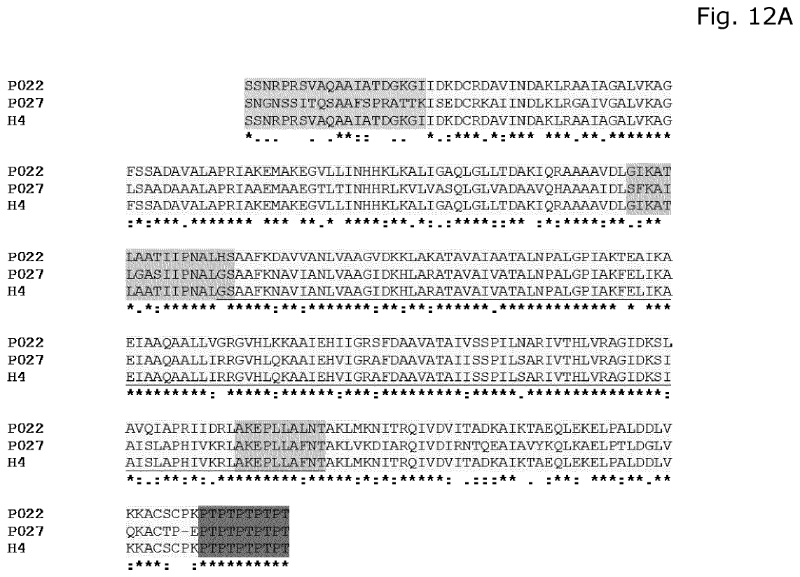

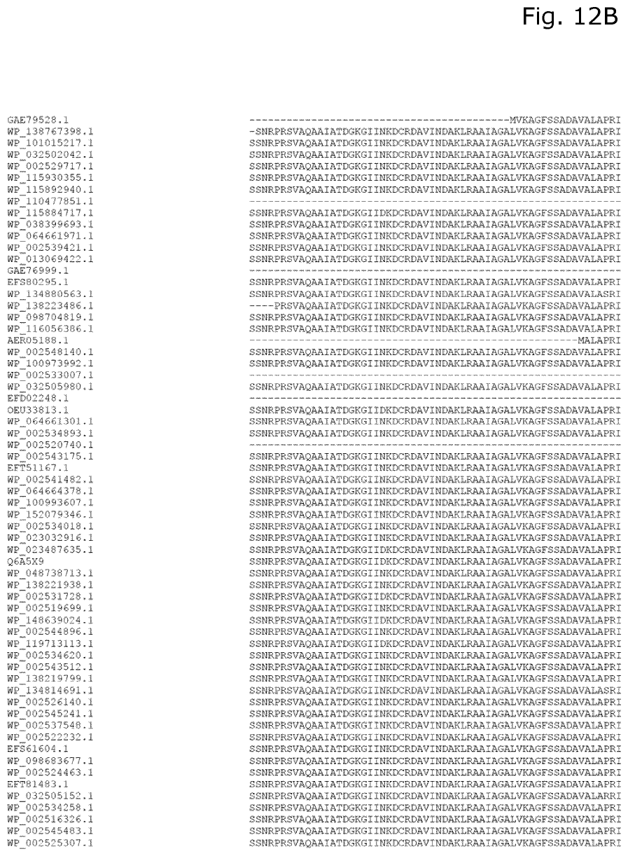

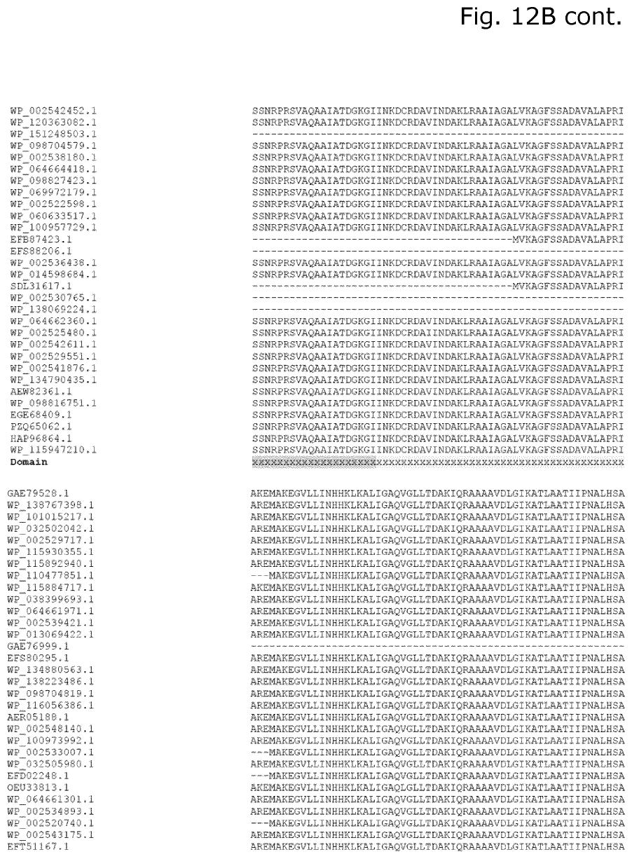

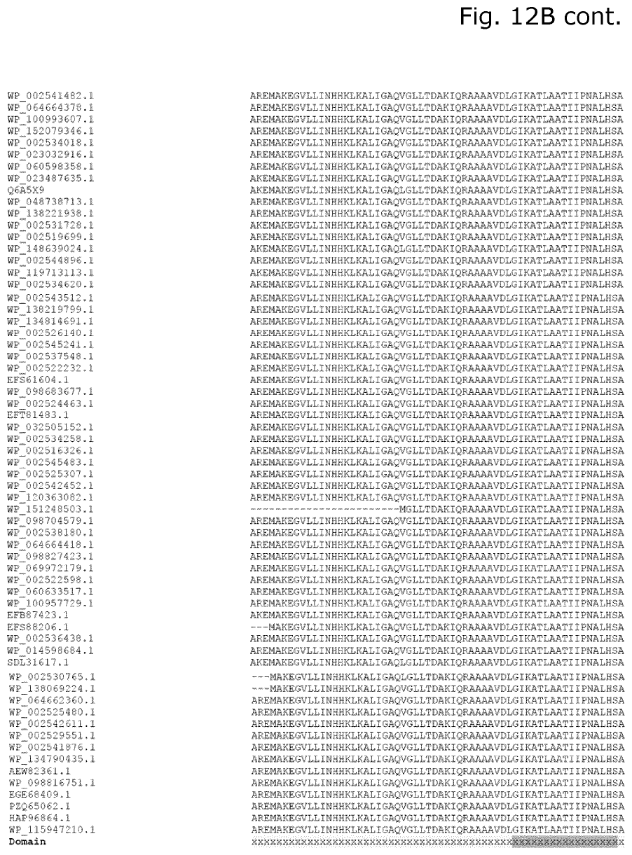

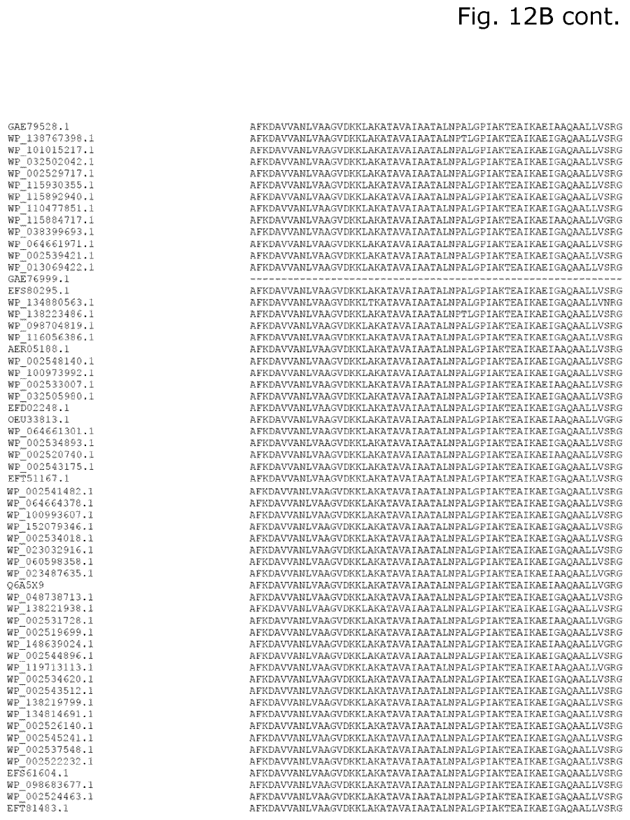

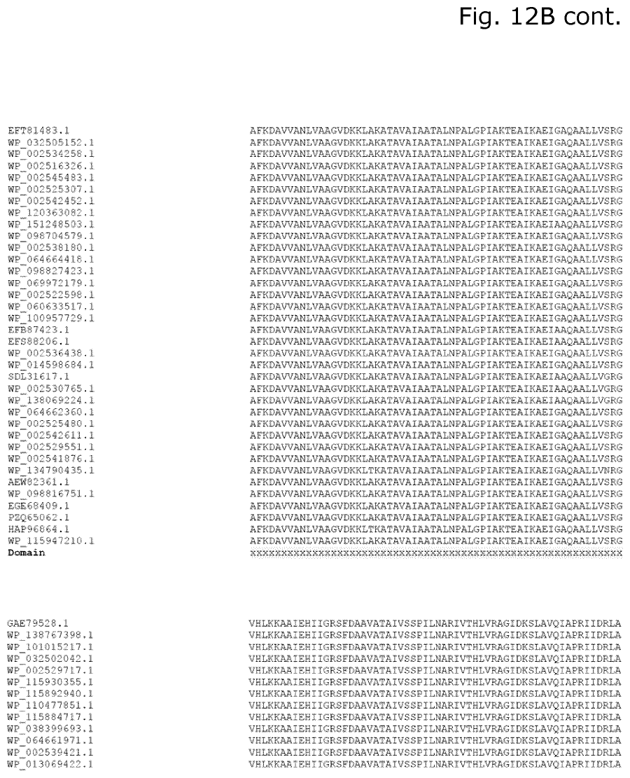

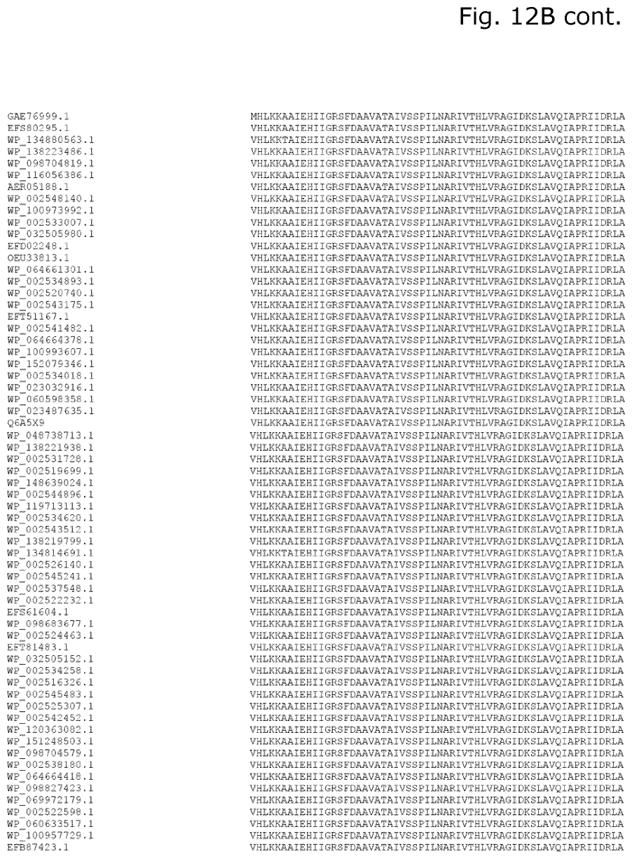

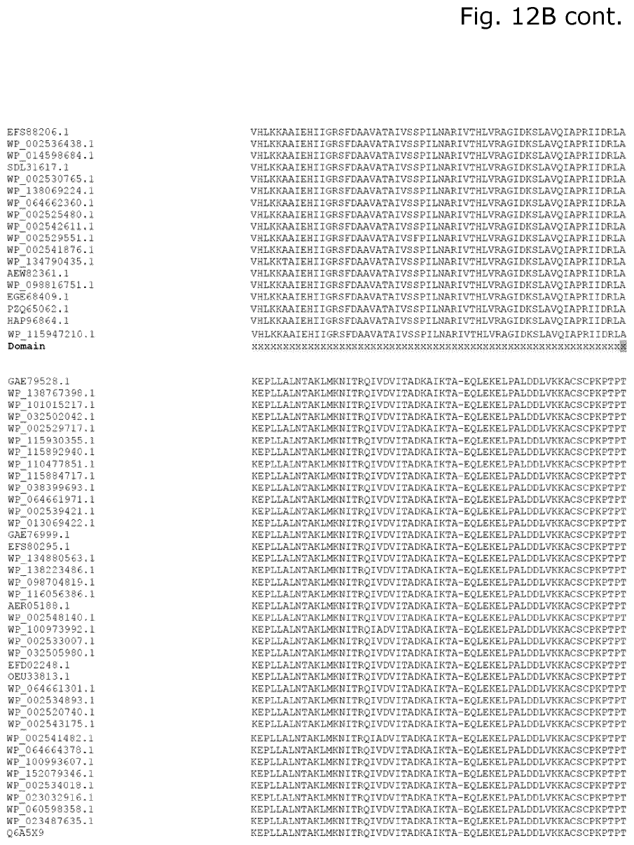

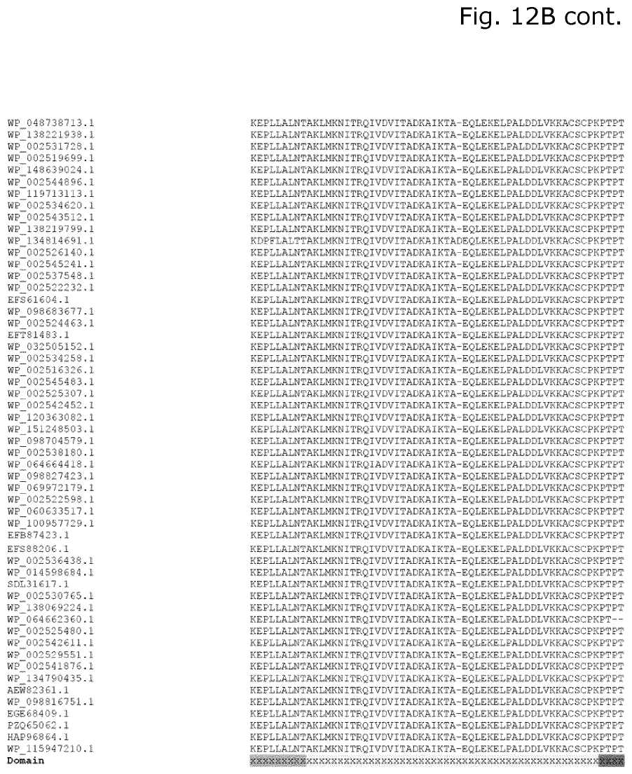

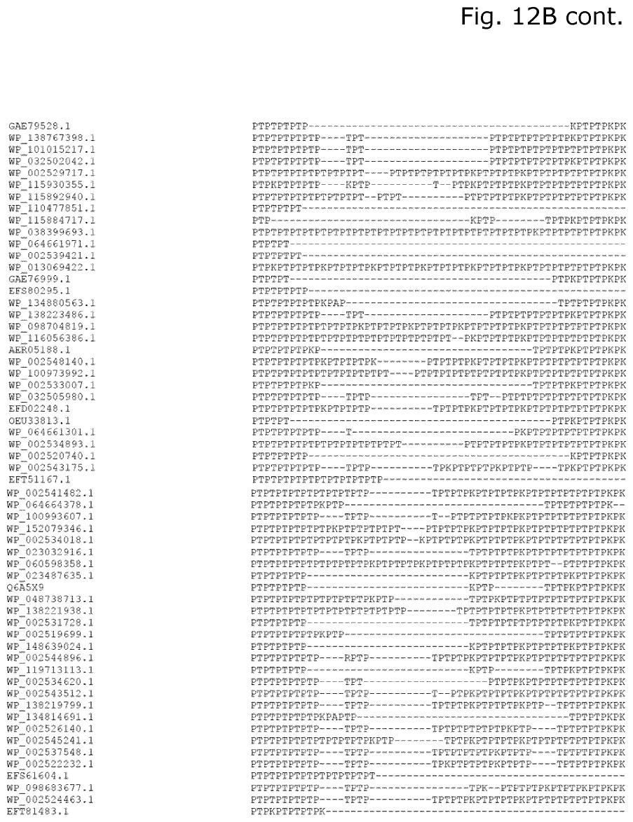

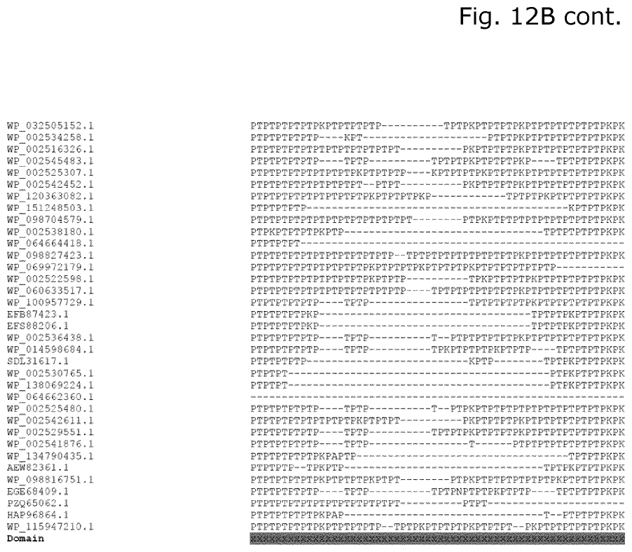

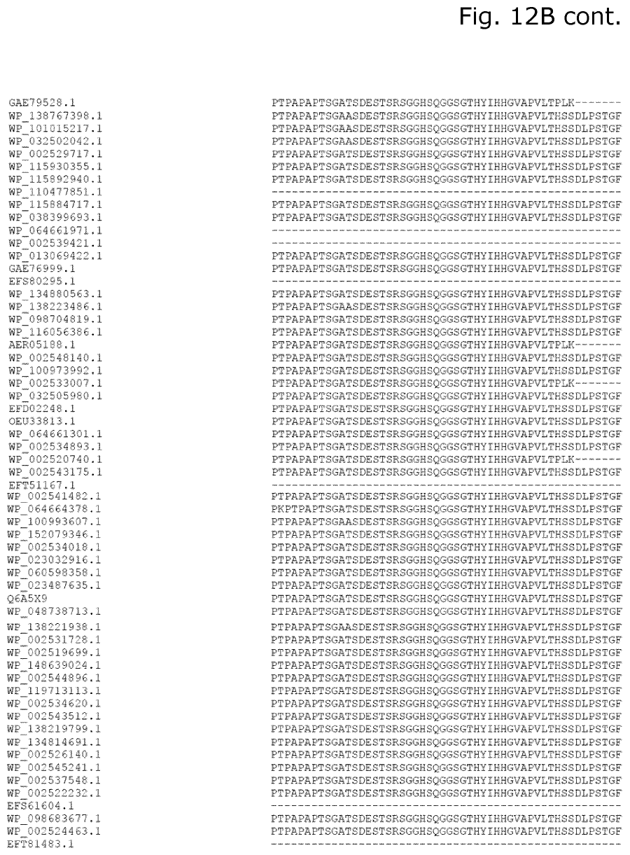

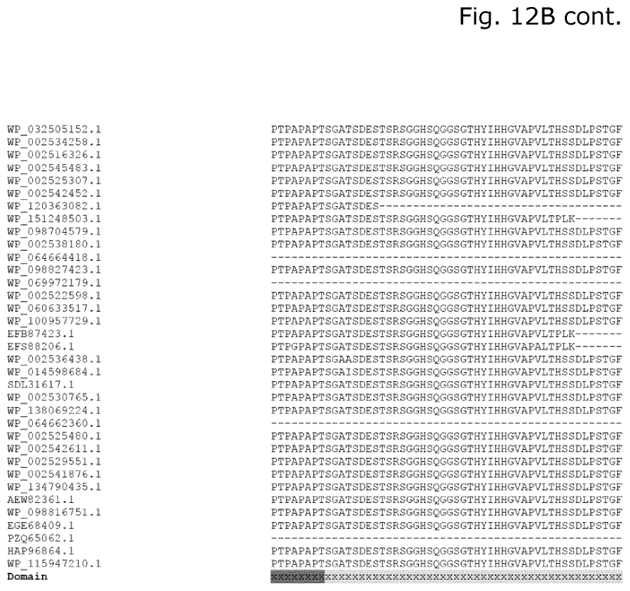

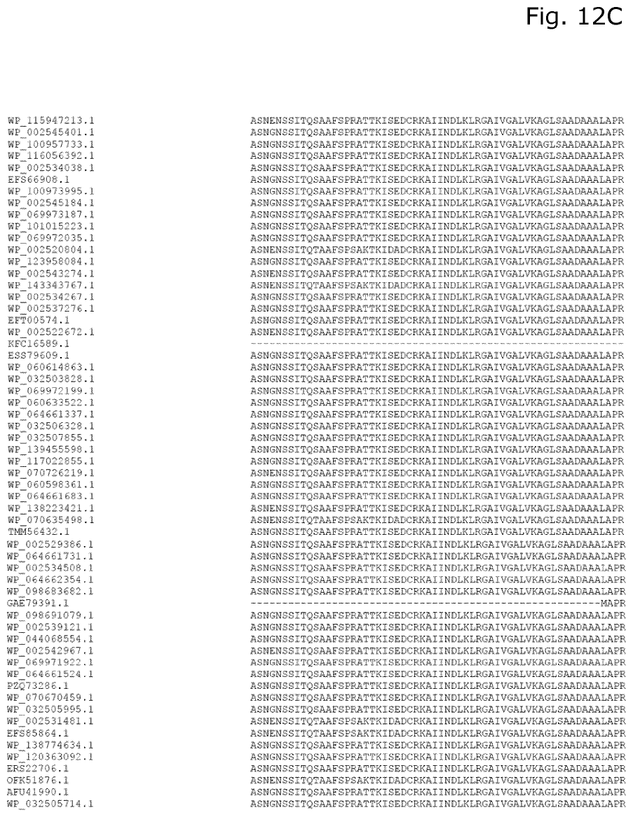

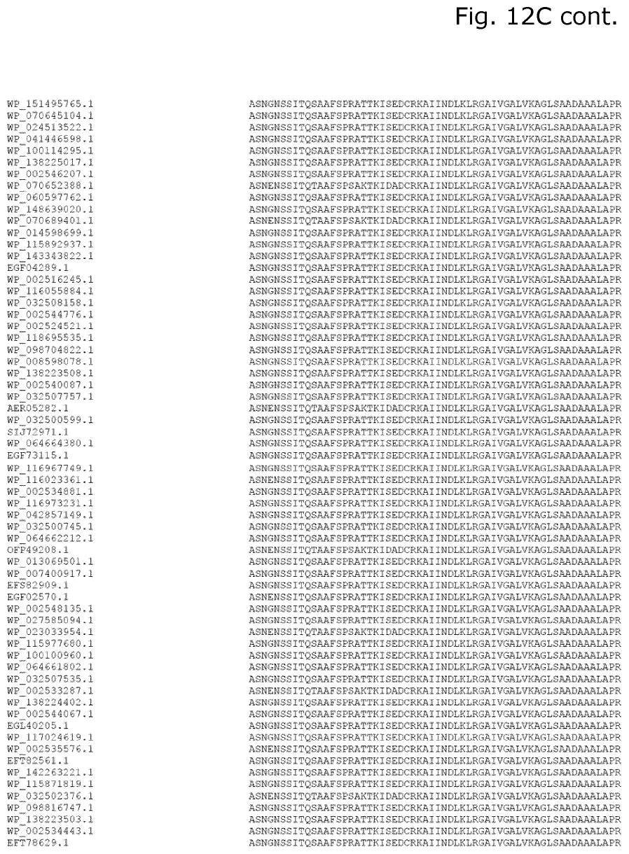

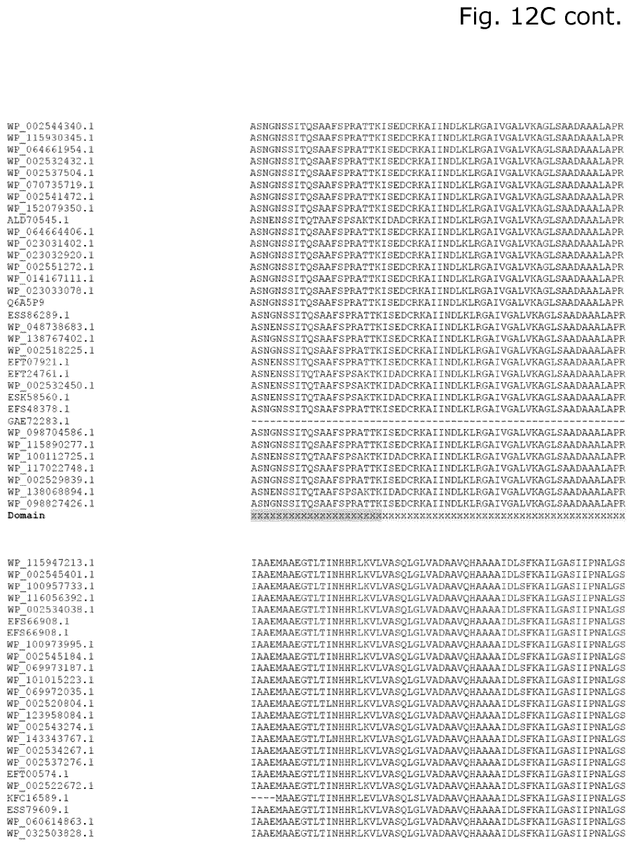

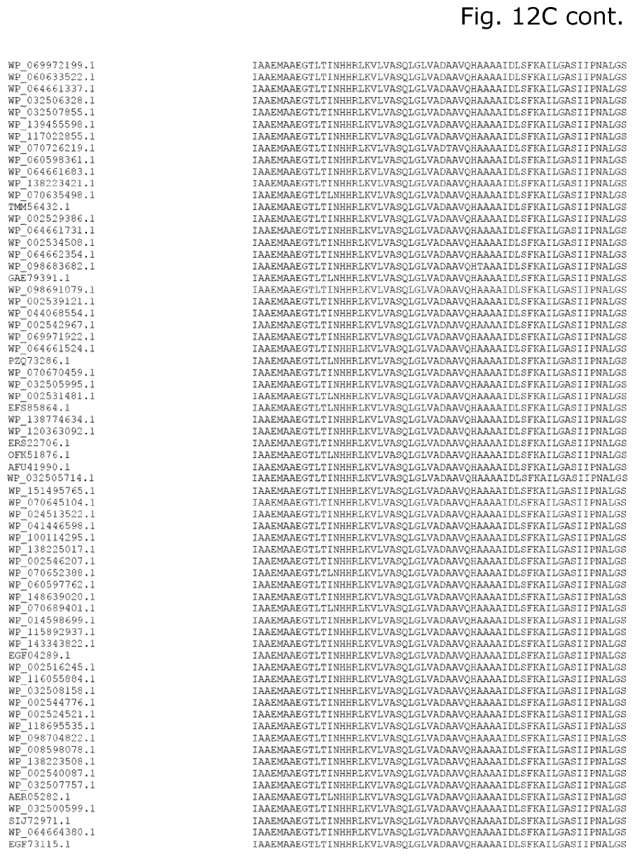

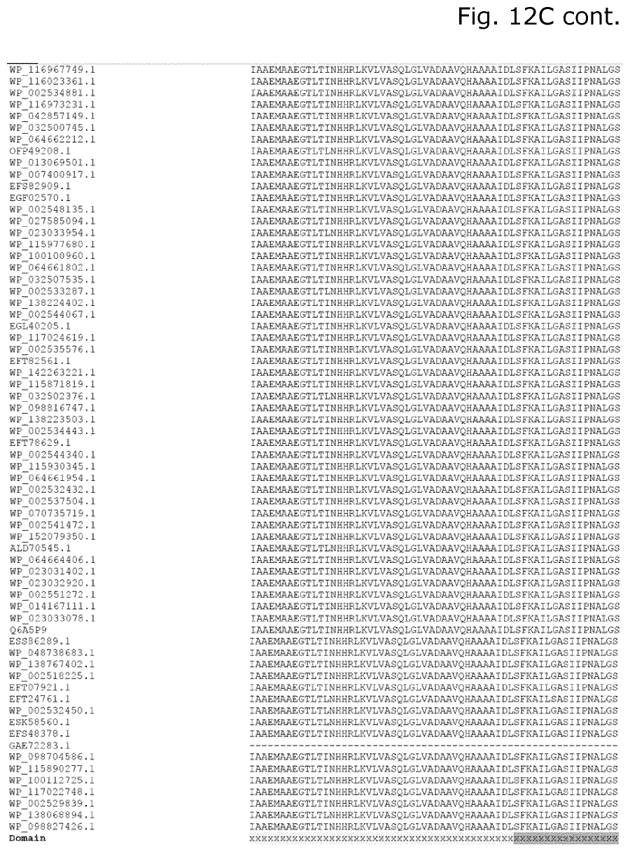

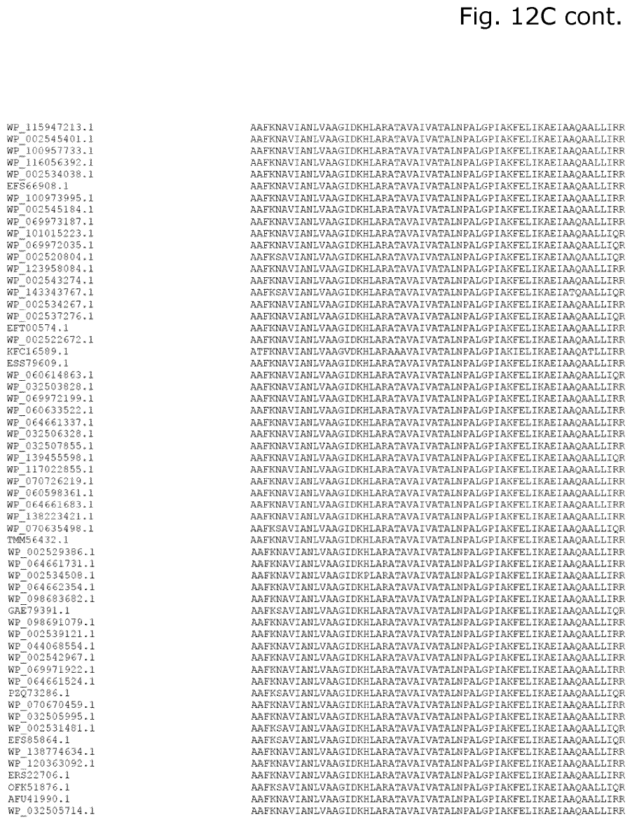

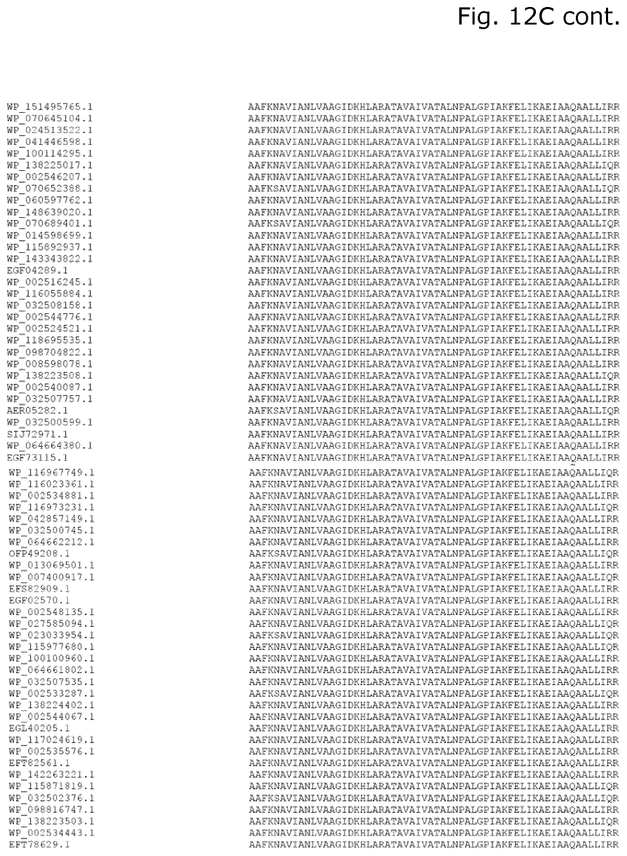

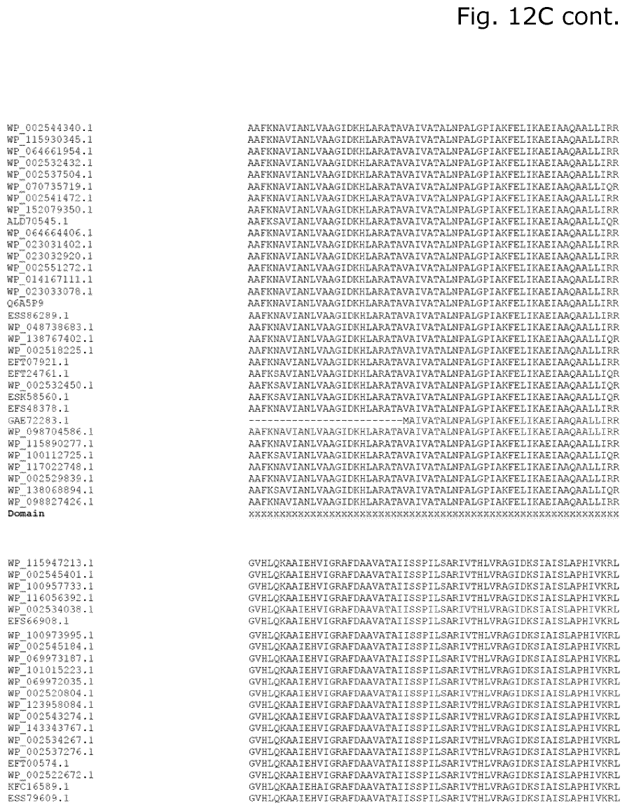

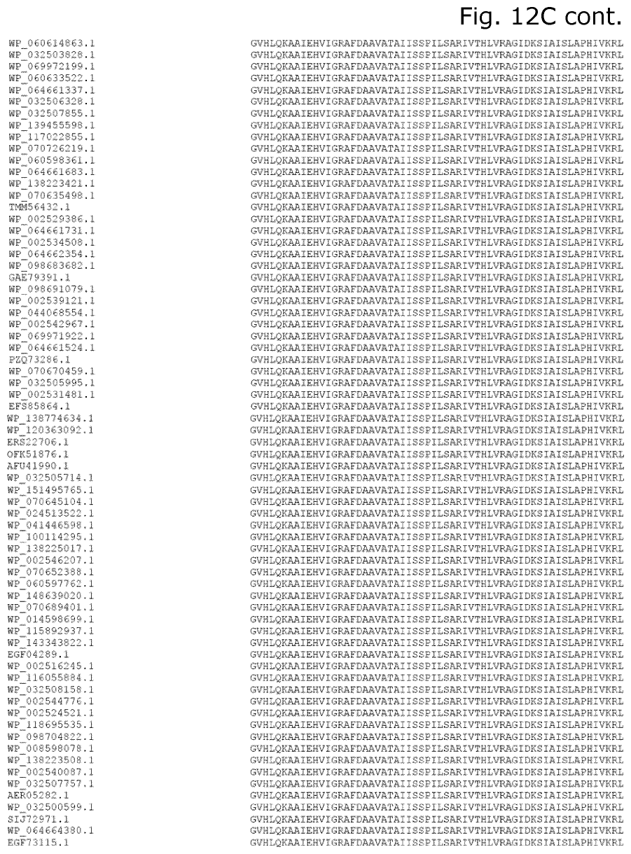

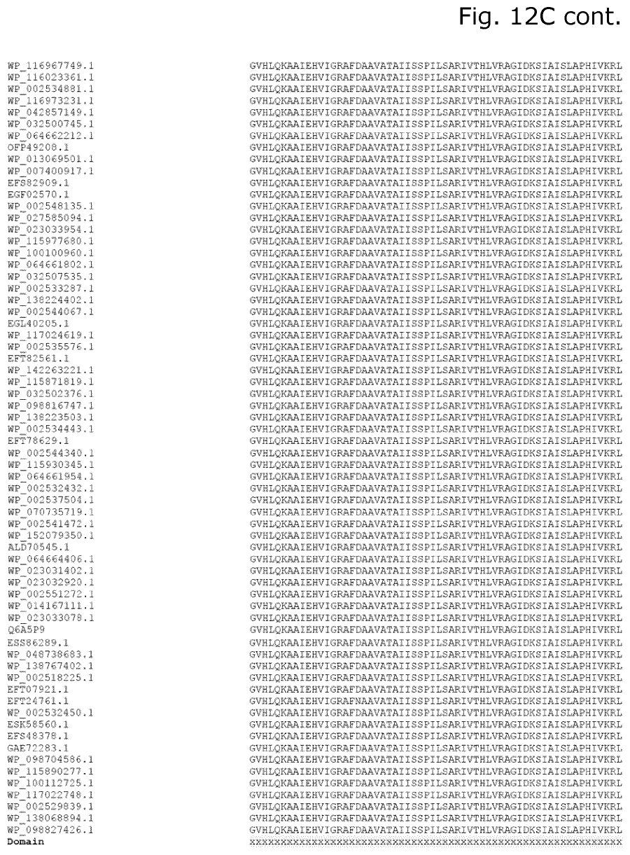

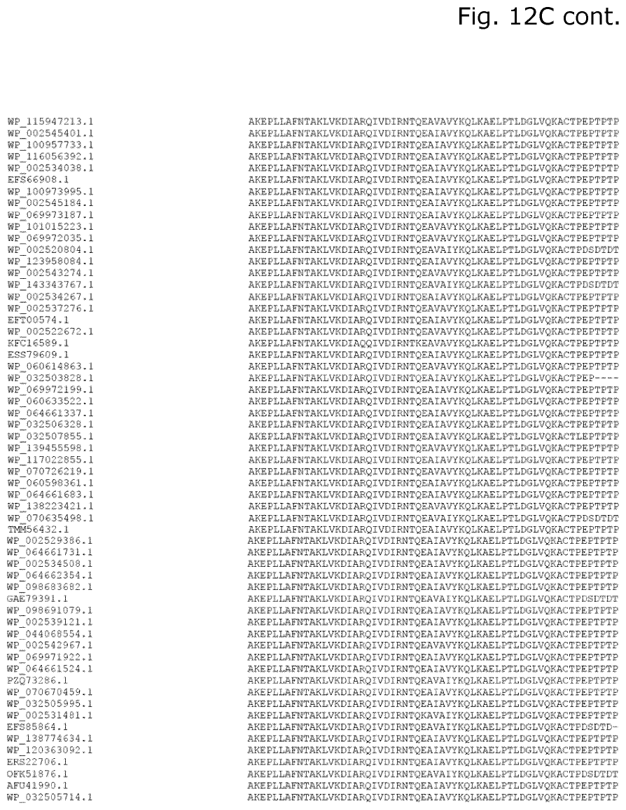

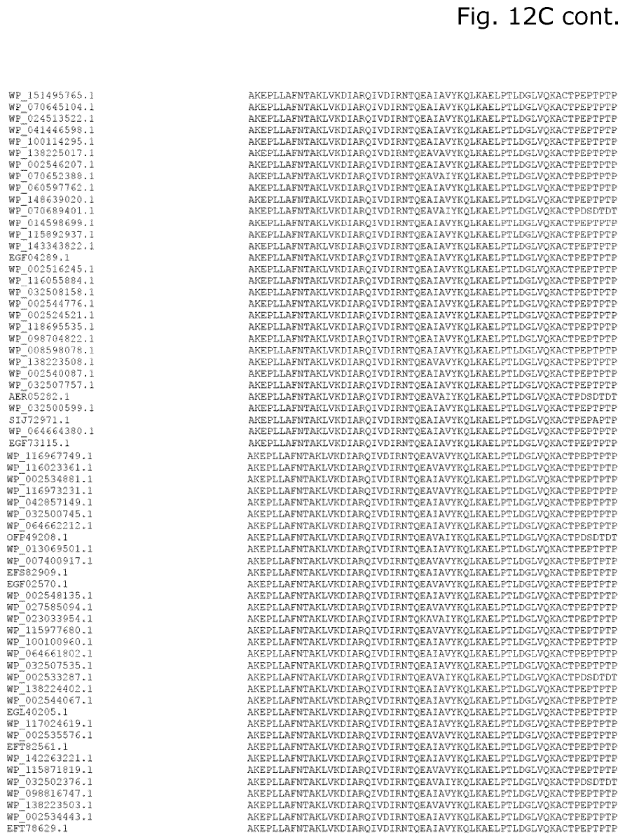

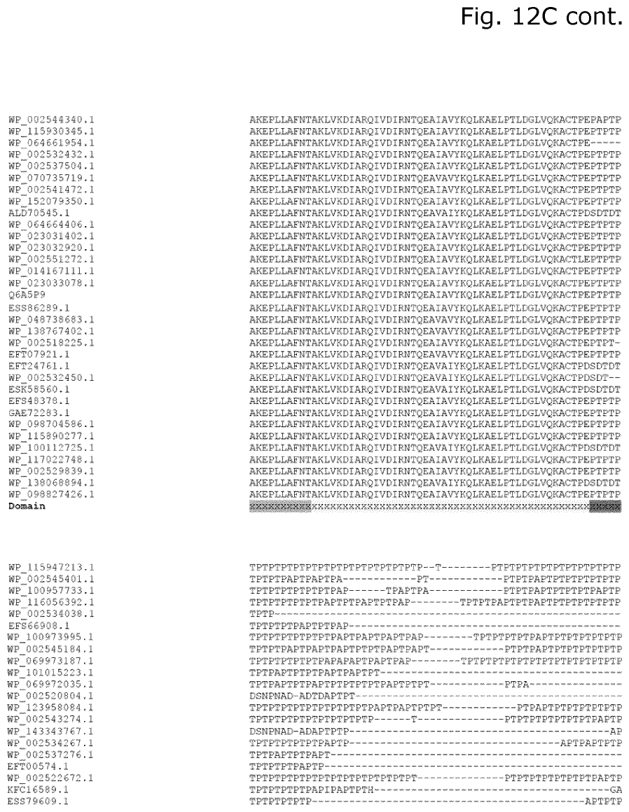

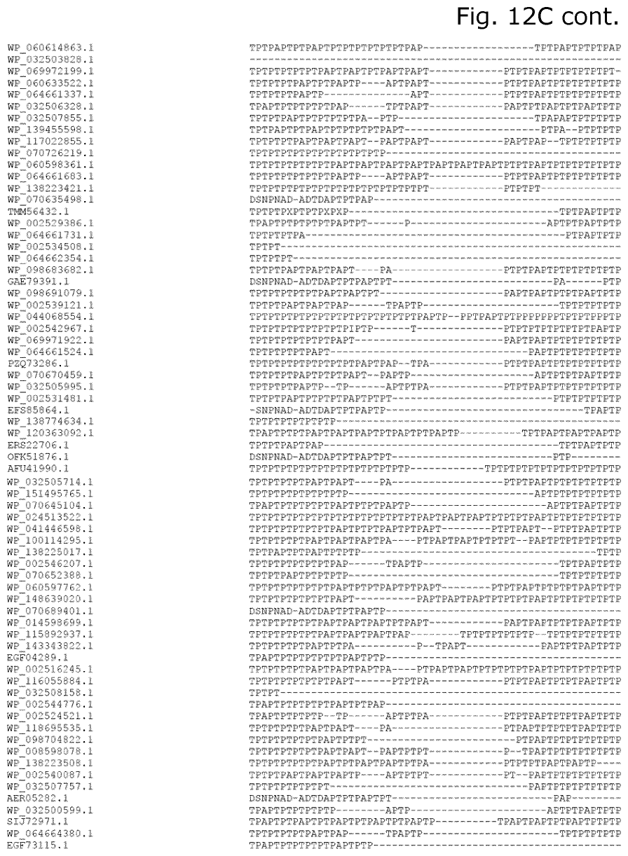

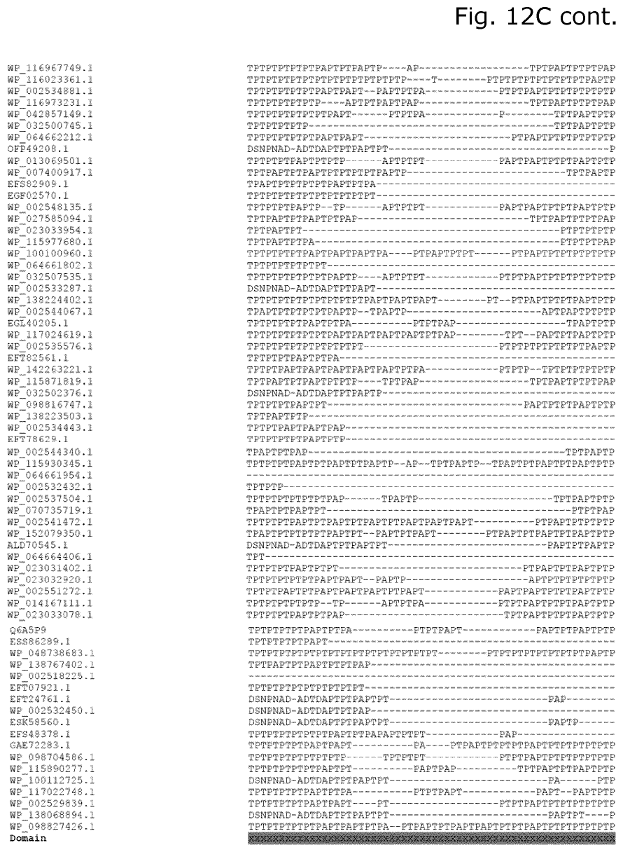

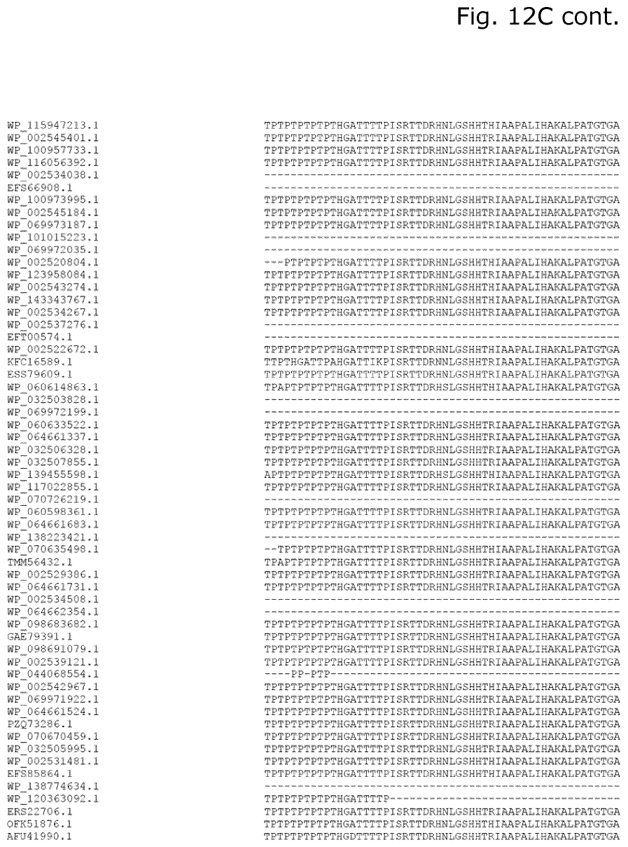

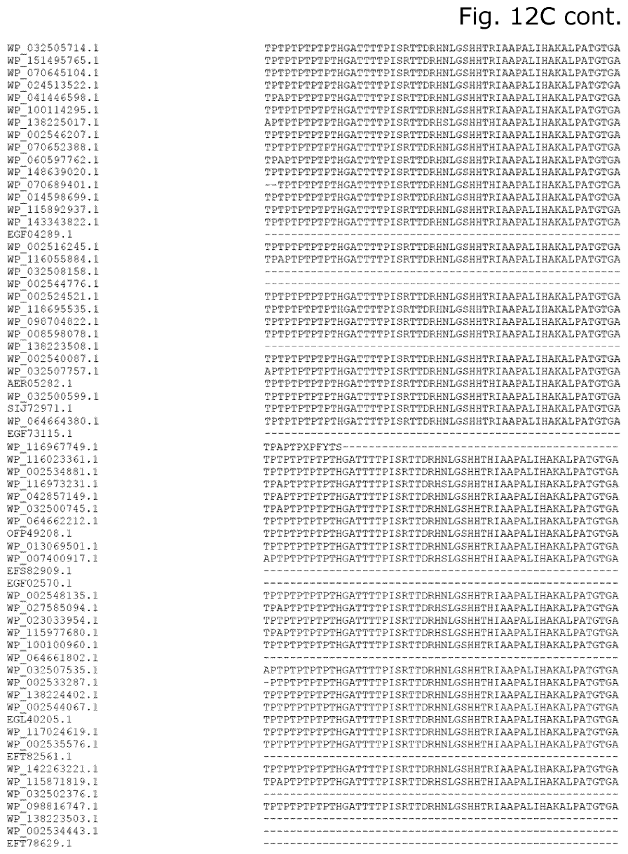

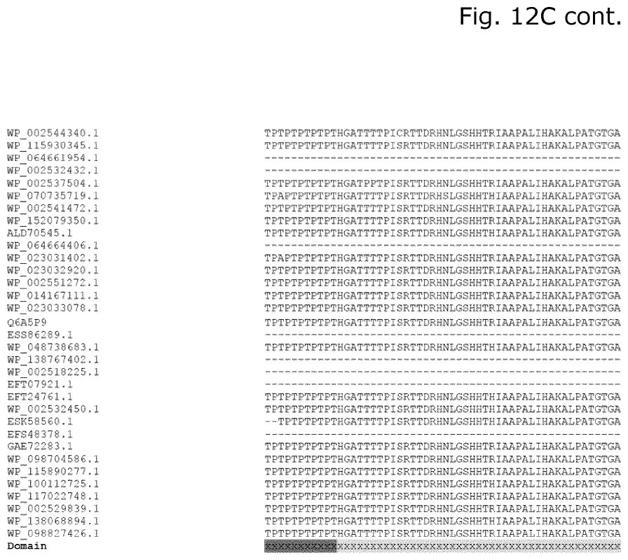

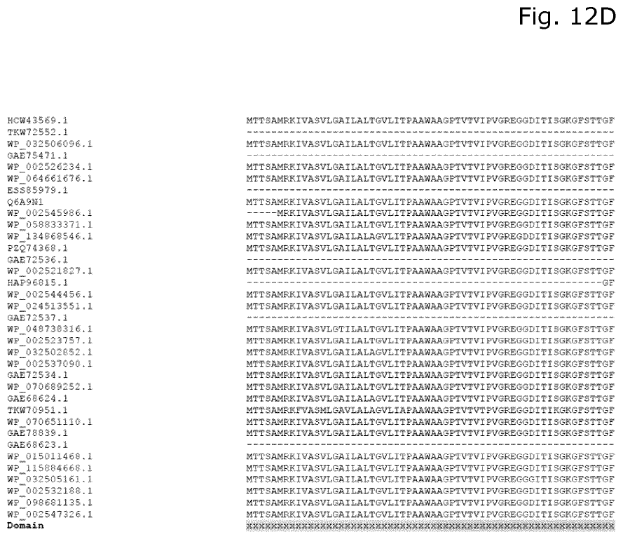

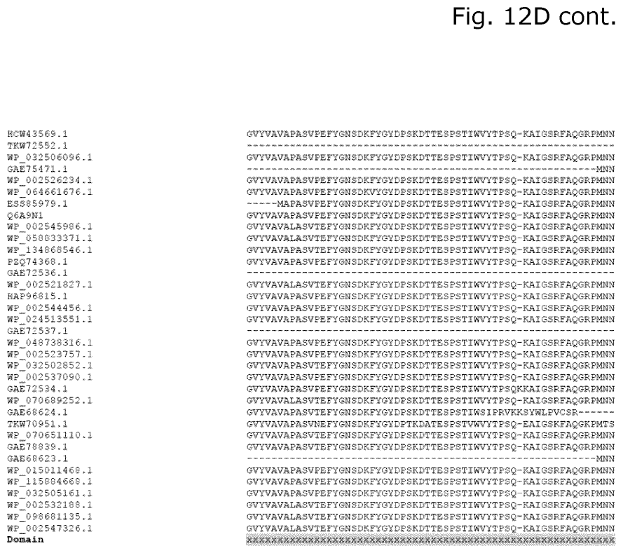

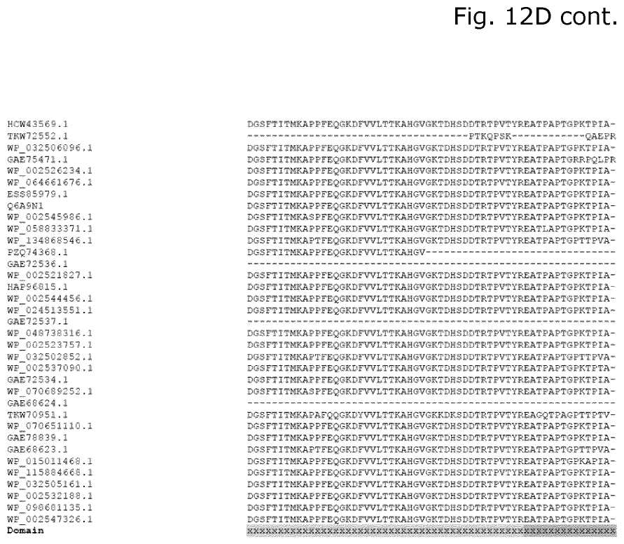

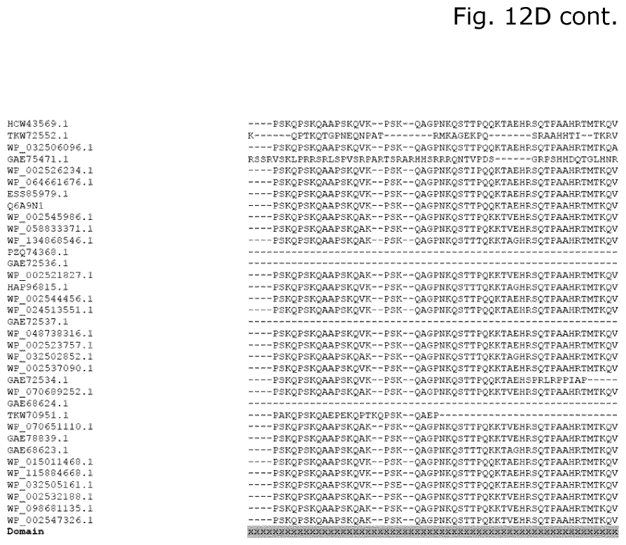

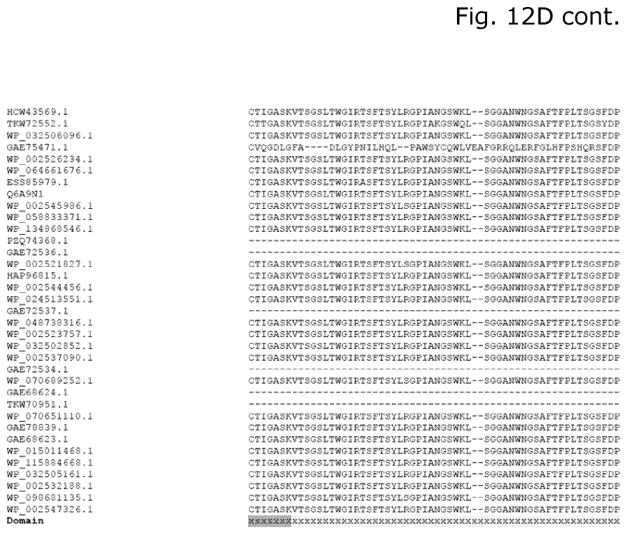

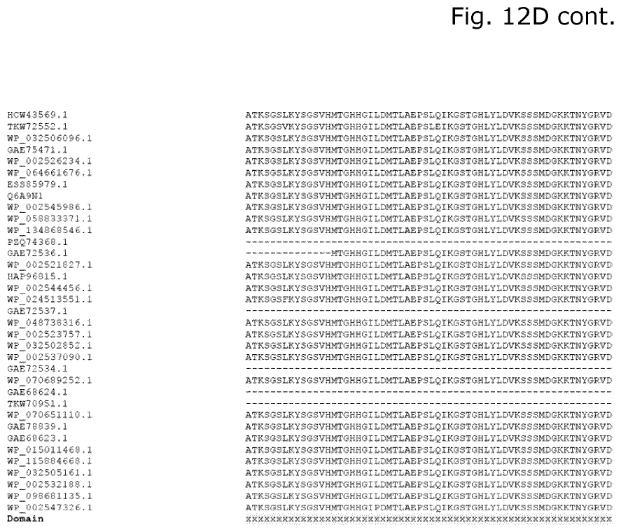

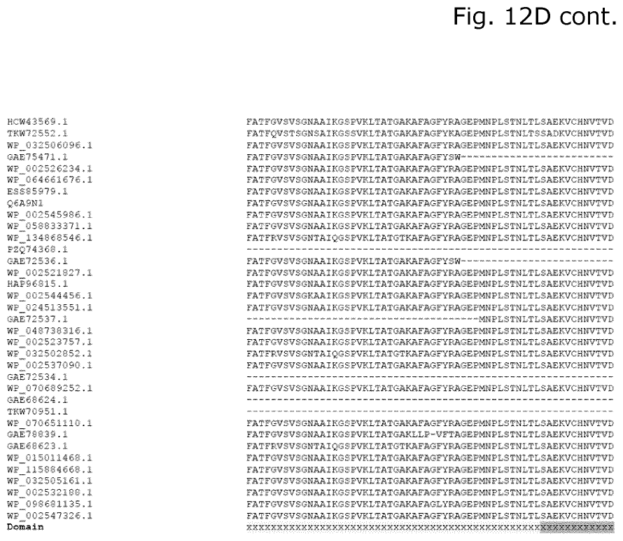

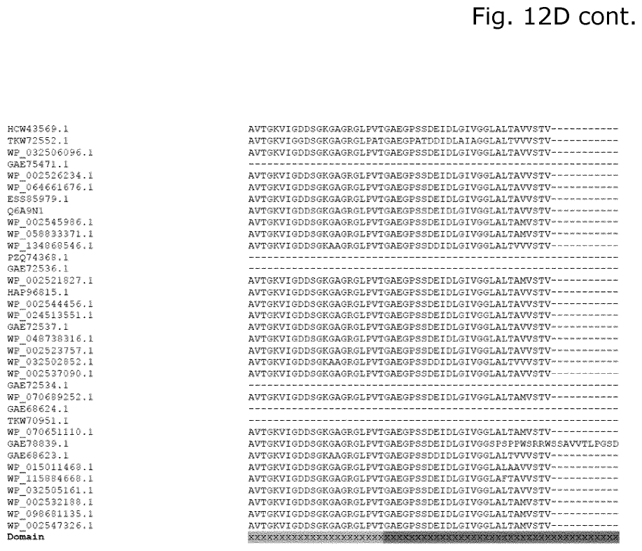

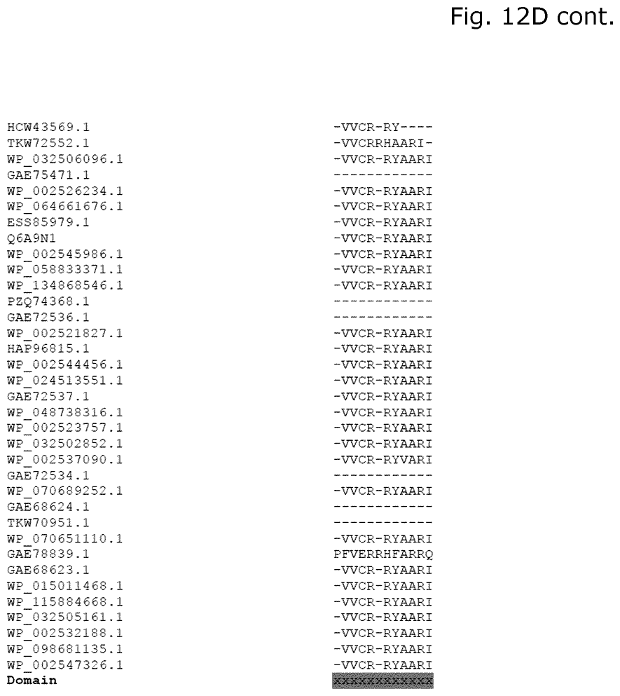

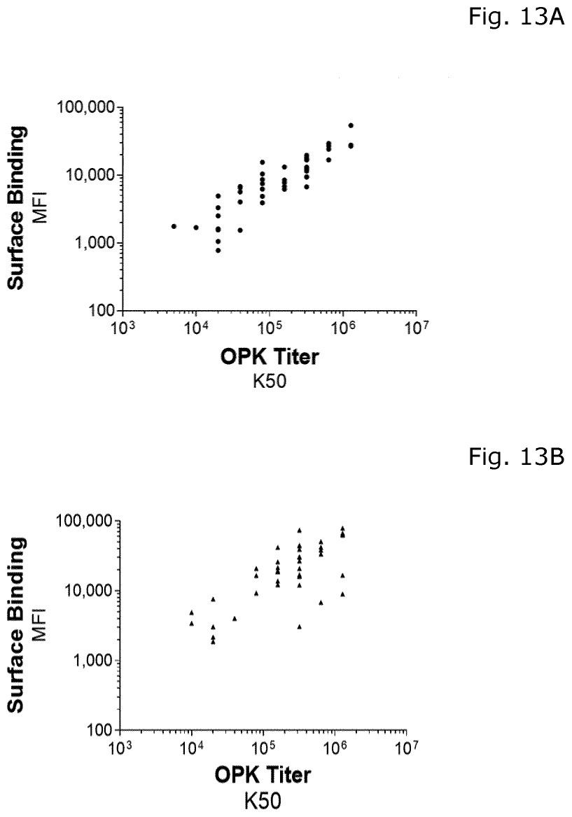

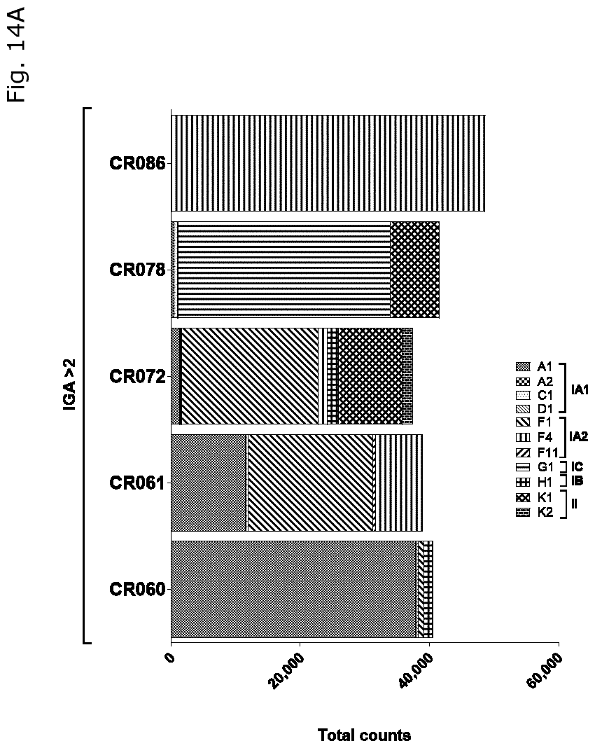

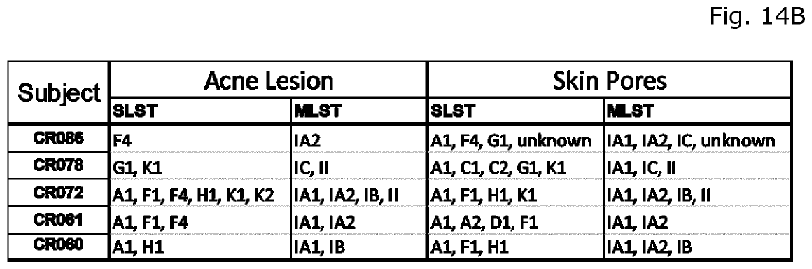

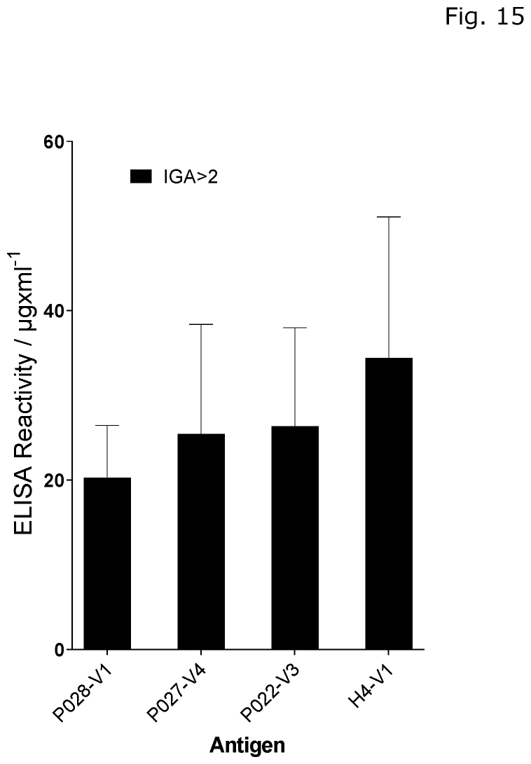

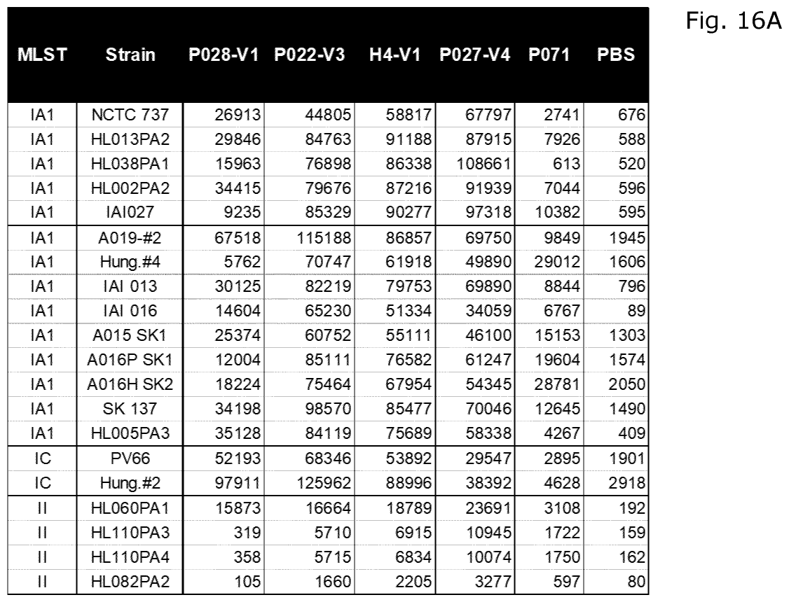

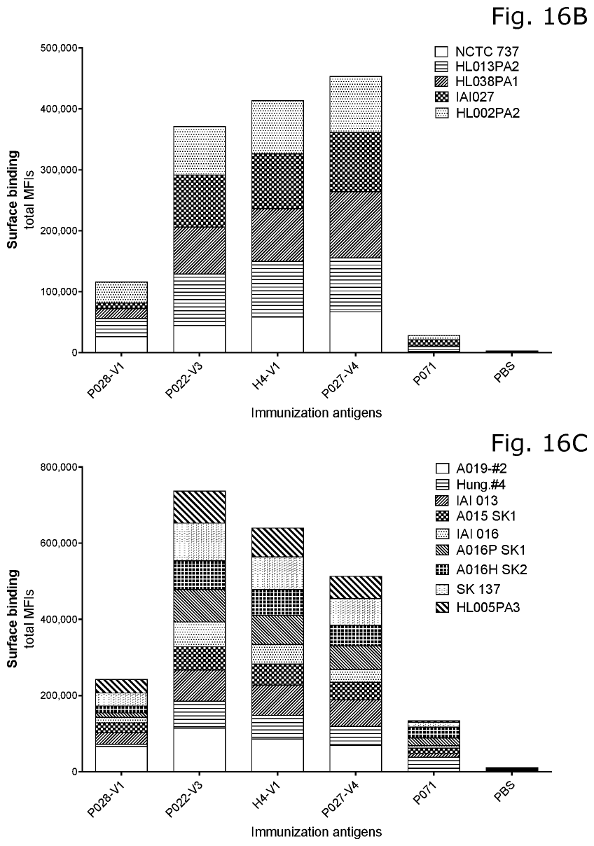

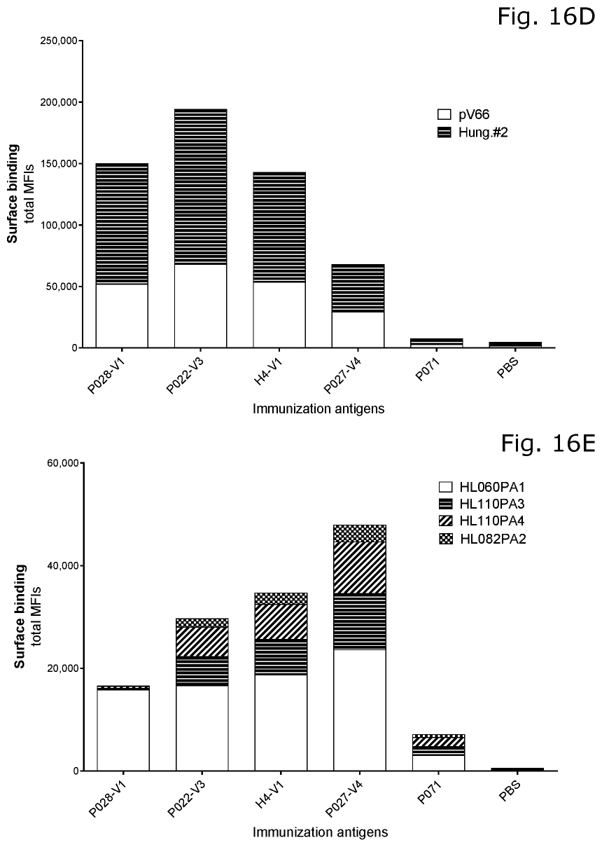

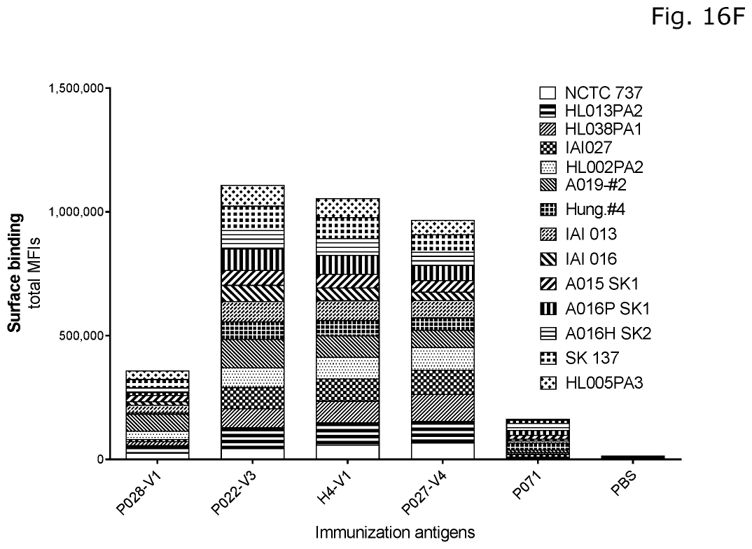

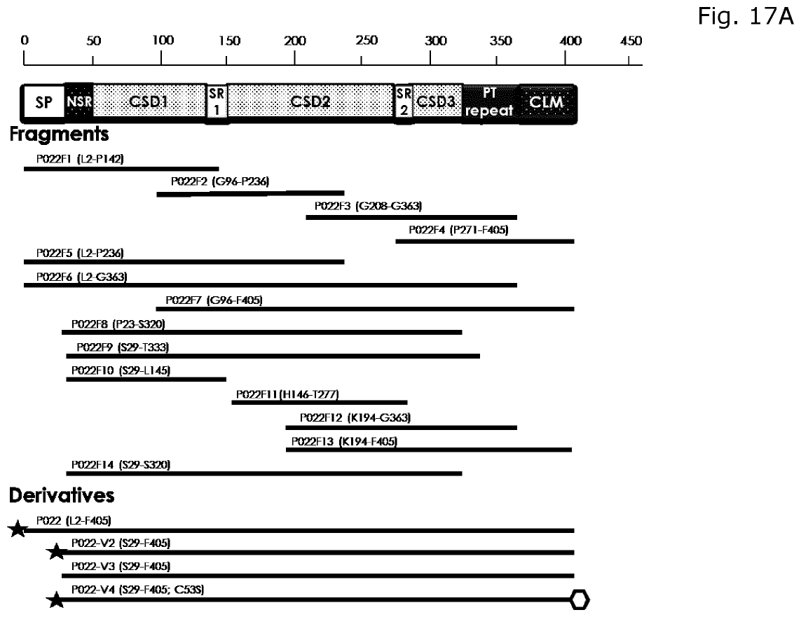

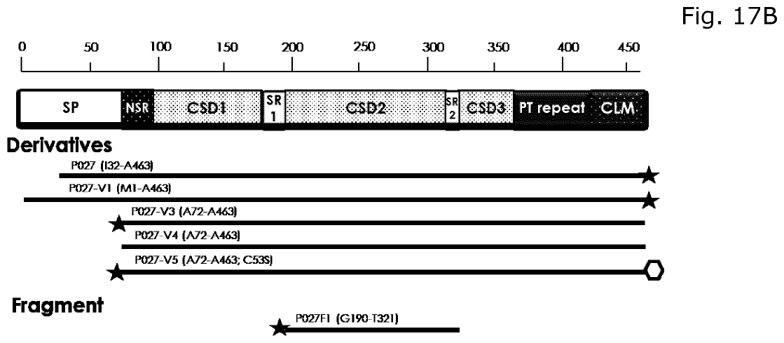

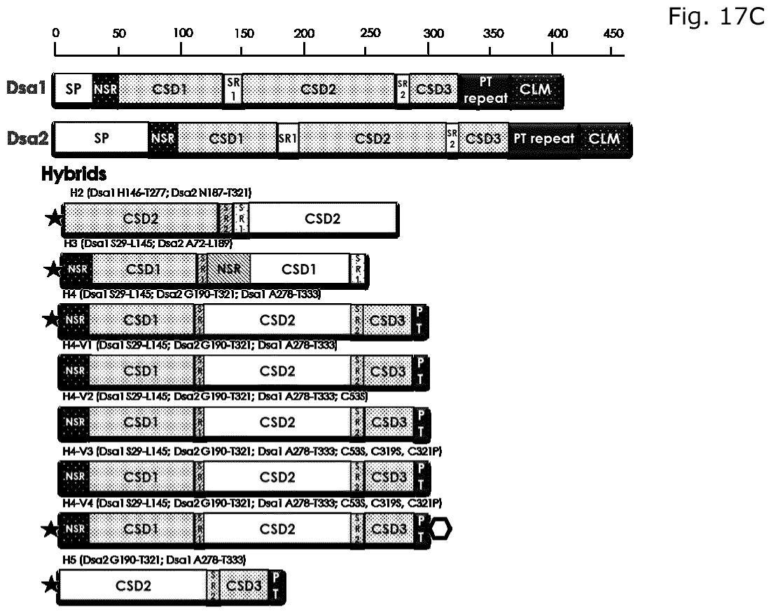

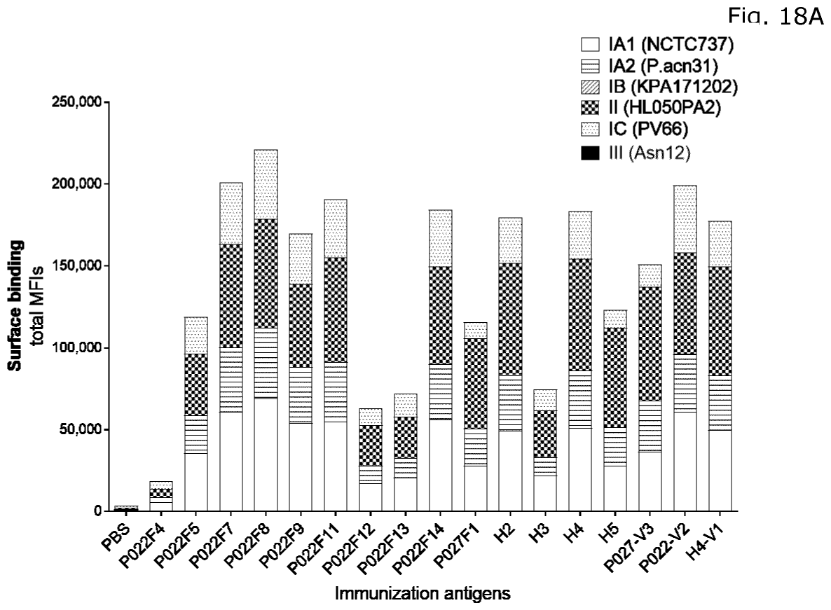

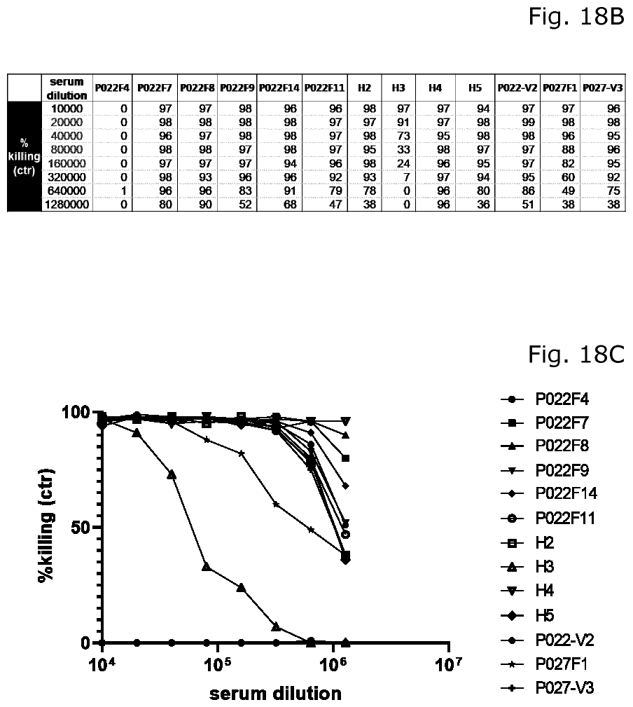

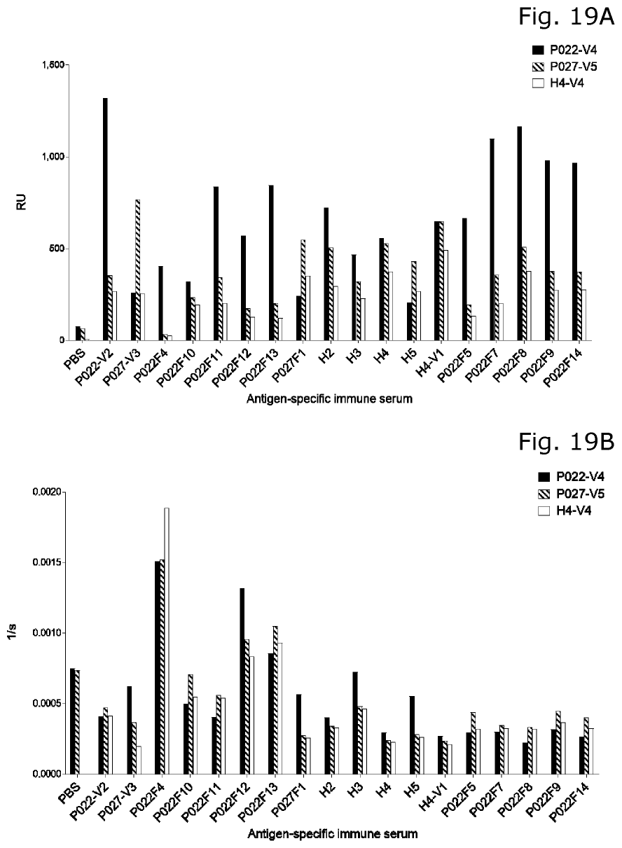

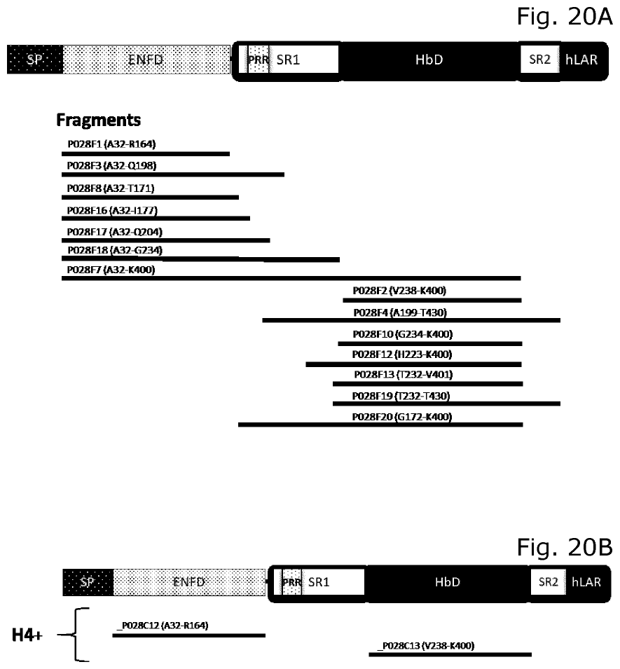

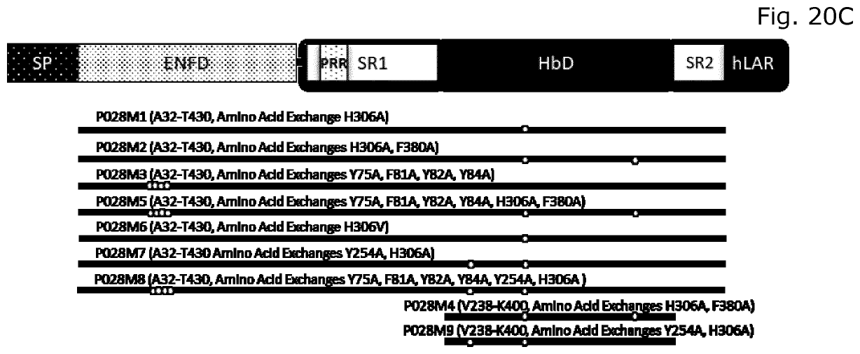

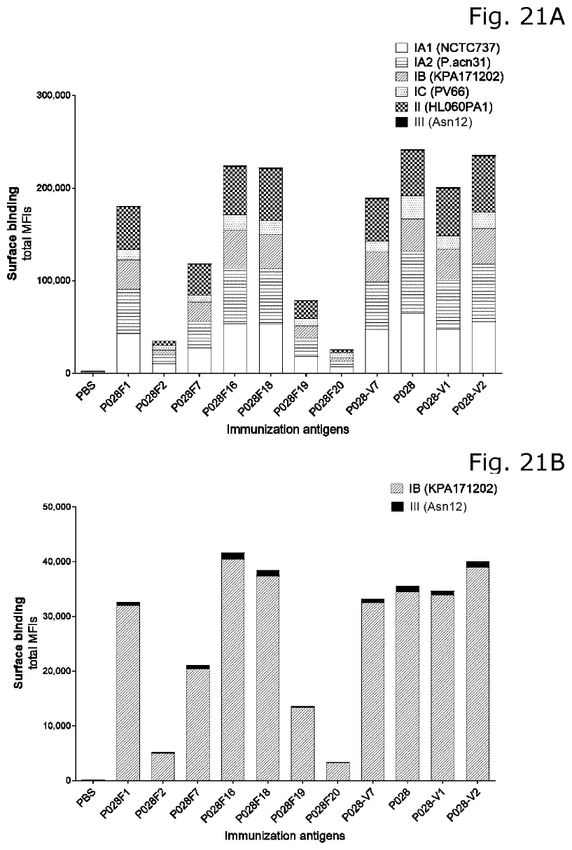

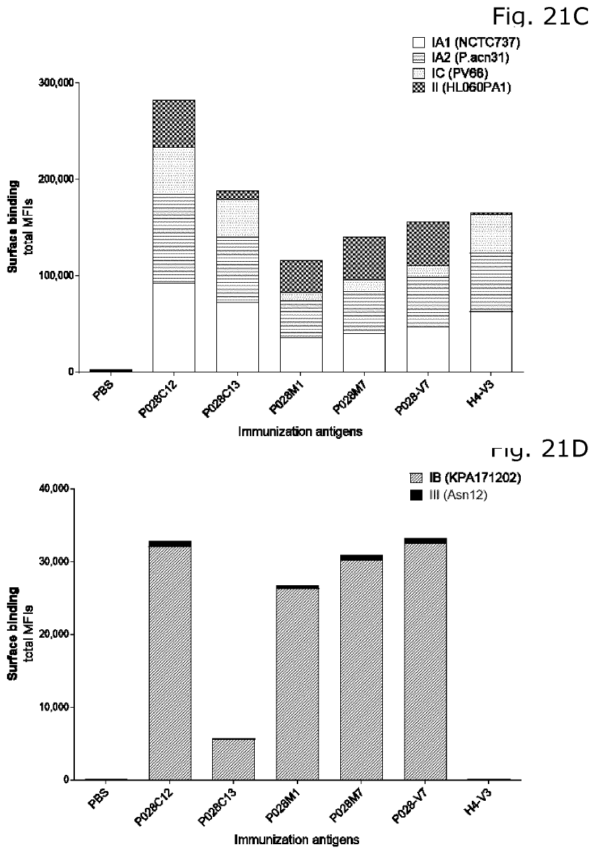

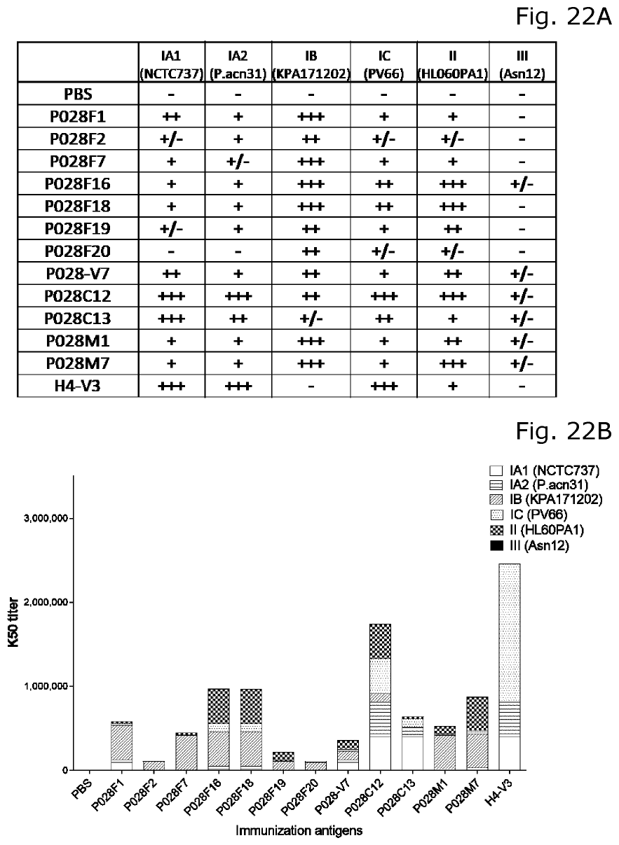

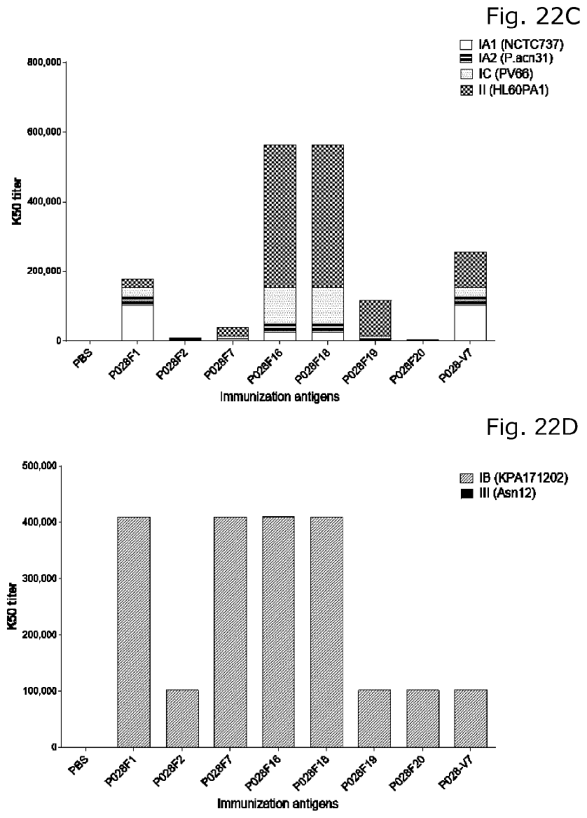

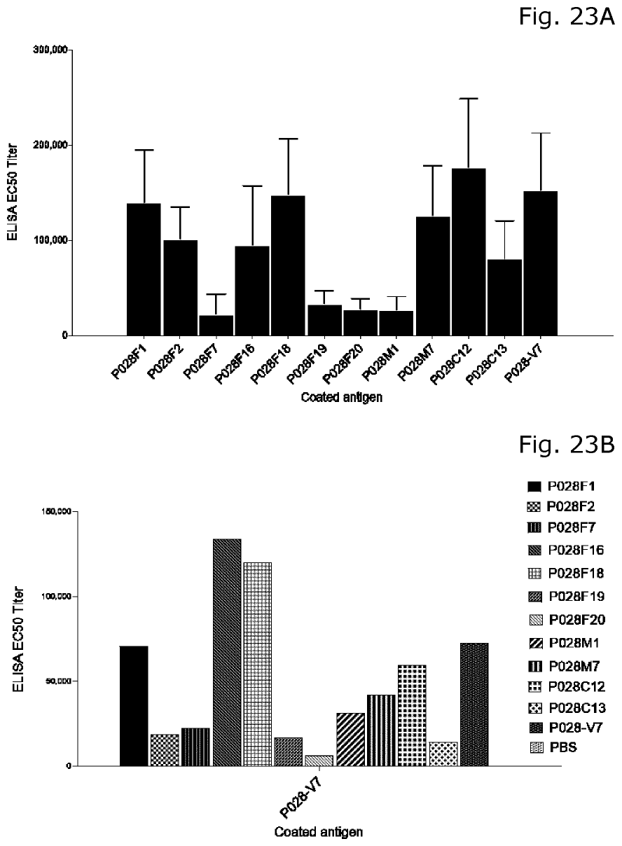

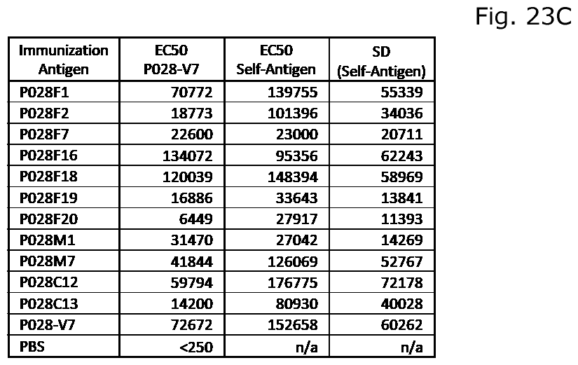

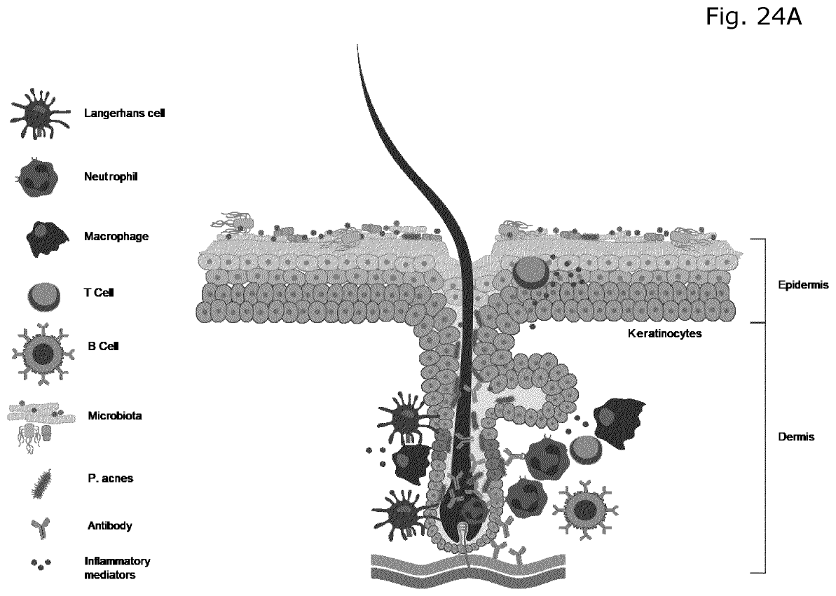

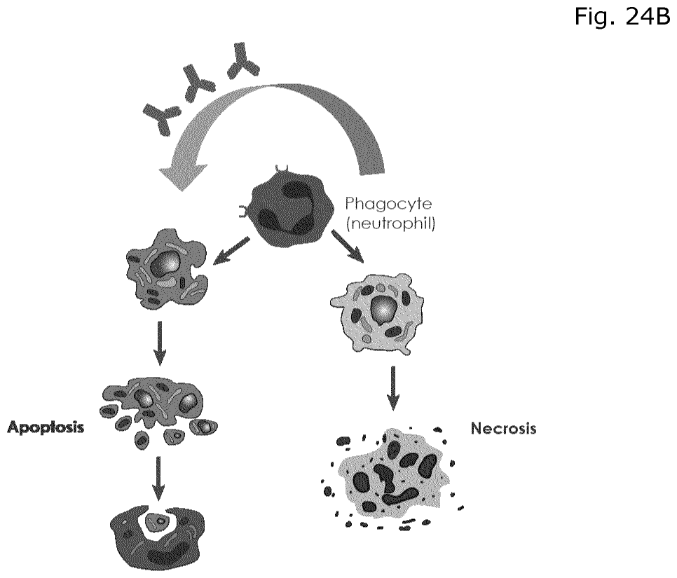

FIG. 1B

Opsonophagocytic killing activity of the antigen-specific murine sera against P. acnes strains from different MLST types. Murine immune sera (n=5) raised against single protein Alhydrogel® formulations of P022-V2 (DsA1), P027-V3 (DsA2), P028 (PITP) and other P. acnes surface antigens (P002, P005, P018, P032, P035, P042, P046, P068, P069, P070 and P071) were tested and compared to negative assay controls. Serial dilutions of murine immune sera were incubated with different P. acnes strains in the presence of human phagocytic cells. Opsonophagocytic killing titer (K50 value), defined as the highest serum dilution leading to more than 50% reduction of viable bacteria have been determined for P. acnes strains of genetic types IA1 (NCTC737), IA2 (P.acn31), IB (KPA171202), IC (PV66), II (HL050PA2) and III (Asn12). Reaction aliquots were plated onto blood agar plates at the beginning and at the end of the experiment. K50 values were ranked in reference to the K50 values of the negative control: K50 values between 500 and 2,500 were regarded as borderline (+/−), values above 2,500 up to 25,000 were marked positive (+), above 25.000 up to 300.000 highly positive (++), and above 300,000 extremely high positive (+++); K50 values below 500 or comparable to the adjuvant immunization alone were considered negative and below the detection limit (−).

FIG. 1C

Graphic representation of the data from FIG. 1B showing the comparison of antigens based on the K50 values of the murine sera against P. acnes strains from the MLST types that express DsA1 and/or DsA2. K50 titer values of different strains are summarized per antigen serum and displayed as total K50 titer (stacked columns).

FIG. 1D

Graphic representation of the data from FIG. 1B showing the comparison of antigens based on the K50 values of the murine sera against P. acnes strains from the MLST types that do not express DsA1 and DsA2. K50 titer values of different strains are summarized per antigen serum and displayed as total K50 titer (stacked columns).

FIG. 2

Serum antibodies induced by immunization with different proteins P022-V2 (DsA), P027-V3 (DsA2), P028 (PITP) and other P. acnes surface antigens (P002, P005, P018, P032, P035, P042, P046, P068, P069, P070 and P071) were investigated by ELISA. Serial dilutions of murine immune sera (n=10) raised by immunization with Alhydrogel® formulations of P022-V2 (DsA1), P027-V3 (DsA2), P028 (PITP) and other P. acnes surface antigens (P002, P005, P018, P032, P035, P042, P046, P068, P069, P070 and P071) were tested to measure the amount of binding to the respective protein antigen (the immunogen) to determine the antibody EC50 titers before (pre-immune) and after 4 immunization rounds (immune, final bleed). Maxisorb Immuno-plates were coated with the respective antigen, then blocked with BSA, incubated with diluted serum samples and detected using TMB and a peroxidase-conjugated goat anti-mouse antibody. OD was measured at 450/620 nm and EC50 values were calculated using GraphPad Prism® software and a four-parameter sigmoid lit.

FIG. 3

Iron influence on the expression of P. acnes proteins on the cell surface. Murine immune sera (n=5, pooled) raised against Alhydrogel® formulations of P. acnes surface proteins P022-V2 (DsA1), P027-V3 (DsA2), P028 (PITP) and other P. acnes surface antigens (P002, P005, P018, P032, P035, P042, P046, P069, P070 and P071) were analyzed in a flow cytometry-based bacterial surface binding assay. Bacteria were cultivated both in the presence and in the absence of the iron-chelator deferoxamine. Cultures of P. acnes strains from the genetic types IA1 (NCTC737), IC (PV66) and III (Asn12) were re-suspended in HBSS buffer containing 2% BSA and incubated with 1:1000 diluted immune sera. Antibody binding was detected with an Alexa Fluor 488 conjugated goat anti-mouse IgG (Fab′)2 fragment. Median fluorescence intensity (MFI) values were measured after cross-linking with 2% PFA by a BD Accuri C6 plus instrumen. Fold changes in specific surface antigen expression levels (MFI in the absence of iron/MFI in the presence of iron) are shown.

FIG. 4

Surface binding of murine immune sera (n=5, pooled) raised against Alhydrogel® formulations of single protein formulations of P022-V3 (DsA), P027-V4 (DsA2), P028-VI (PITP) or adjuvant Alhydrogel® (PBS), as a negative control. 60 different P. acnes strains were tested and grouped according to the 16S ribotype classification scheme developed by Fitz-Gibbon (Fitz-Gibbon et al. 2013). Bacteria were cultivated in the presence of the iron-chelator deferoxamine. Cultures of different P. acnes strains were re-suspended in HBSS buffer containing 2% BSA and incubated with 1:1000 diluted immune sera. The bound antibodies were detected with an Alexa Fluor 488-conjugated goat anti-mouse IgG Fab′)2 fragment. Median fluorescent intensity (MFI) values were measured after cross-linking with 2% PFA on a BD Accuri C6 plus instrument. The strain-specific MFI values of different immune sera were grouped and depicted as columns for P. acnes ribotypes 1, 2, 3, 4, 5, 6, 8, 9, 16 and 532. Ribotypes 4, 5, 8 are related to acne, whereas ribotypes 2 and 6 are predominately found on healthy skin; ribotypes 1 and 3 are found on both acne and healthy skin (Fitz-Gibbon et al. 2013; McLaughlin et al. 2019).

FIG. 5

Binding of murine serum antibodies generated by antigen immunizations to the cell surface of P. acnes strains from the representative genetic types. Murine immune sera (n=5, pooled) raised against Alhydrogel® formulations of P028-VI (PITP), P022-V3 (DsA1), P027-V4 (DsA2) and combinations of P022-V3+P027-V4 (DsA1+DsA2), P028-V1+P022-V3 (PITP+DsA1), P028-V1+P027-V4 (PITP+DsA2), P028-V1+P022-V3+P027-V4 (PITP+DsA1+DsA2) were analysed in a flow cytometry based bacterial surface binding assay. Bacteria were cultivated in the presence of the iron-chelator deferoxamine. Cultures of P. acnes strains belonging to different MLST groups (McDowell et al. 2012; Barnard et al. 2015) genetic types IA1 (NCTC737), IA2 (P.acn31), IB (KPA171202), IC (PV66), II (HL050PA2) and III (Asn12) were re-suspended in HBSS buffer containing 2% BSA and incubated with 1:1000 diluted immune sera. Binding antibodies were detected with an Alexa Fluor 488 conjugated goat ani-mouse IgG (F′ab′)2 fragment. Median fluorescence intensities (MFI) were measured after cross-linking with 2% PFA on a BD Accuri C6 plus instrument. MFIs of different strains were summarized per serum and displayed as total MFI's.

FIG. 6

Binding of rabbit serum antibodies generated by immunizations with DsA1 and fragments thereof to the cell surface of P. acnes strains from the representative genetic types. Rabbit pre-immune and immune sera raised against oil-in-water emulsions of P022 (DsA1; mean n=3) and P022F1 to P022F4 fragments were analyzed in a flow cytometry-based bacterial surface binding assay and compared to an immune serum induced by vaccination with NCTC737 whole bacterial cells (RWS). Cell cultures of P. acnes strains belonging to different MLST groups (McDowell et al. 2012; Barnard et al. 2015): types IAI (NCTC737), IA2 (P.acn31), IB (KPA171202), II (HL050PA2) and III (Asn12) were re-suspended in HBSS buffer containing 2% BSA and incubated with 1:1000 diluted immune sera. Binding of antibodies was detected with an Alexa Fluor 488 conjugated goat anti rabbit IgG F′ab′)2 fragment. Median fluorescent intensities (MFI) were measured after cross-linking with 2% PFA on a BD Accuri C6 plus instrument. The intensity of the binding of individual antigen-specific immune sera is expressed as median fluorescence intensity (MFI) derived after subtracting MFI value obtained with the corresponding pre-immune serum. Bacterial strain specific reactivities are grouped to columns for each immunization antigen. Rabbit serum raised against P. acnes whole bacterial cells was used as a positive control (RWS) and diluted 1:32,000, because of the strong signal intensity and the detection limit of the Flow cytometer.

FIG. 7A

3D modelling of DsA1 Protein with the structural domains indicated. Conserved domains 1-3 (CSD1 to 3) were modelled as three helix bundles based on the putative structural homologue S. aureus Extracellular matrix-binding protein ebhA (PDB ID 2DGJ). CSD2 comprises two bundles which share one helix. Between CSD1 and CSD2 as well as CSD2 and CSD3 are unstructured or partially structured regions. The extent of the ‘swap regions’ was approximated using creation of a large number of models, taking the ten best and considering conservation of helices vs. extended loop. Swap regions were therefore used as safe sites for separating CSD domains. For creating hybrids, specifically H4 and H5, swap regions are the sites within which a ‘cross-over’ between homologues takes place. Due to the high degree of homology between DsA1 and DsA2 there are few gaps, and structures can be thought to be fairly similar. Yet, it was considered as being the safest strategy to take functionally and structurally more or less independent CSD domains from an individual protein and combine it with the CSD of the homologue. The sequence areas where these switches between sequences could be most safely positioned, were marked as the “swap regions”. This way the risk of creating aberrant structure was minimized. NSR and PT repeat are mostly missing from the structure.

FIG. 7B

The sequence of hybrid H4 in alignment with source sequences P022 (DsA1) and P027 (DsA2), with a focus on the domain junctions. Shown is DsA1 CSD1 where it joins to DsA2 CSD2, as well as DsA2 CSD2 where it joins to DsA1 CSD3. The respective swap regions, SR1 and SR2 (essentially alternative preferred sites for placing a sequence cross-over between DsA1 and DsA2) are marked in grey.

FIG. 7C

Because P022 and P027 are homologues the text for FIG. 7A applies here entirely as well.

FIG. 7D

The ENFD domain of PITP has been modelled based on Neocarzinostatin, a chromoprotein Streptomyces macromomyceticus. Neocarzinostatin is thereby a small-molecule binding protein with a specificity for enediyne antibiotics, where the role of the protein is to stabilize and detoxify the cargo until its delivery. Sequence and structural analysis shows significant similarity between ENFD and Neocarzinostatin-like proteins, but also significantly extended loops (shown in green) extending from the shared core beta-sheet structure (shown in blue) in the ENFD compared to its homologue. Based on Neocarzinostatin and other related proteins of resolved structure shared characteristics of ligands and the ligand binding site can be defined, and the presented figure shows a likely fit of hemin (red stick model) into this modelled ENFD binding site. Residues with significant potential to coordinate heme iron are shown as orange stick models.

FIG. 7E

The HbD domain of PITP has been modelled based on a moderately close homologue, the HtaA heme-binding domain of Corynebacterium glutamicum. While overall sequence similarity is only average, heme contacting and coordinating residues are highly if not full conserved. The presented model shows the protein (blue) in complex with heme (red), where selected residues are highlighted as orange stick models. Specifically, axial ligand Tyr254 and closely associated (affinity contributing) His306 are shown, as well as two other residues contributing to specific and affine binding of heme.

FIG. 8

Cross-binding of antibodies induced by immunization with different fragments and hybrids of P022-V2 (DsA1) and P027-V3 (DsA2) investigated by ELISA. Serial dilutions of murine immune sera (n=10) raised by immunization with Alhydrogel® formulations of P022-V2 (DsA1), P022 (DsA1) fragments F9-F13, P027-V3 (DsA2), P027 (DsA2) fragment P027F1, and hybrids (H2-H5) were tested to measure the amount of binding to the full length P022-V2 (DsA1) and P027-V3 (DsA2) proteins. Maxisorb Immuno-plates were coated with P022-V2 (DsA1) and P027-V3 (DsA2), then blocked with BSA, incubated with diluted serum samples and detected using TMB and a peroxidase conjugated goat anti-mouse antibody. OD was measured at 450/620 nm and EC50 values were calculated using GraphPad Prism® software and a four-parameter sigmoid fit. EC50 values are shown as columns for the different immunization antigens.

FIG. 9

Binding of murine serum antibodies generated by antigen immunizations to the cell surface of P. acnes strains from the representative genetic types. Murine immune sera (n=5) raised against Alhydrogel® formulations of single protein formulations of P022-V2 (DsA1), fragments of P022 (F9 to F13), P027 (DsA2), a fragment of P027 (P027F1). P028 (PITP), as well as hybrid constructs (H2-H5) were analysed in a flow cytometry based bacterial surface binding assay. Bacteria were cultivated in the presence of iron-chelator deferoxamine. Cultures of genetic types IA1 (NCTC737), IA2 (P.acn31), IB (KPA171202), IC (PV66), II (HL050PA2) and III (Asn12) were re-suspended in HBSS buffer containing 2% BSA and incubated with 1:1000 diluted immune sera. Antibody binding was detected with an Alexa Fluor 488-conjugated goat anti mouse IgG (F′ab′)2 fragment. Median fluorescent intensities (MFI) were measured after cross-linking with 2% PFA by a BD Accuri C6 plus instrument. Individual strain-specific MFI values are grouped and depicted as columns for each immunization antigen. The MFI values of the sera against H5, which were tested in a separate experiment, were expressed and compared in reference to the internal assay controls.

FIG. 10A

Stability assessment of P022-V3 (DsA1), P027-V4 (DsA2) and H4-V1. 200 ng of the protein solutions have been loaded on SDS-PAGE and have been visualized by silver staining. The gel lanes are marked according to the specific stability test performed: protein samples were submitted to 3 freeze/thaw cycles (FT), stored 7 days at −20° C. (−20), 2-8° C. (+4), room-temperature 21-24° C. (RT) and 37° C. (+37) and have been compared to freshly reconstituted samples (T0) by SDS-PAGE. The Page-Ruler Protein Marker was loaded in the adjacent gel lanes (MW) and bovine serum albumin was loaded as an additional control (BSA standard).

FIG. 10B

Comparison of different constructs that had been manufactured and analyzed under reducing and non-reducing conditions. The following proteins have been loaded on SDS-PAGE and have been visualized by silver staining: P028-V1, P028-V1′ (Expressed by Biomay, purified under denaturing conditions), P028-V1″ (expressed by Biomay, purified under 2-8° C.), P028-V7 (C201S, C372S); H4-V1, H4-V1′ (expressed by Biomay), H4-V2 (C26S), H4-V3 (C26S, C292S, C249P), Page-Ruler Protein Marker has been loaded in the adjacent gel lanes (MW).

FIG. 11A

Binding of murine serum antibodies generated by antigen immunizations to the cell surface of P. acnes strains from the representative genetic types. Murine immune sera (n=5) raised against Alhydrogel® formulations of single protein formulations of P028-V1 (PITP), hybrid H4-V1 (H4-V1) and a protein combination formulation of P028-V1 and hybrid H4-V1 (P028-V1+H4-V1) were analyzed in a flow cytometry based bacterial surface binding assay and compared to a negative control serum raised by the immunization with Alhydrogel® adjuvant (PBS). Bacteria were cultivated in presence of the iron-chelator deferoxamine. Cultures of genetic types IA1 (NCTC737), IA2 (P.acn31), IB (KPA171202), IC (PV66), II (HL050PA2) and III (Asn12) were re-suspended in HBSS buffer containing 2% BSA and incubated with 1:1000 diluted immune sera. Antibody binding was detected with an Alexa Fluor 488-conjugated goat anti-mouse IgG (F′ab′)2 fragment. Median Fluorescent intensities (MFI) were measured after cross-linking with 2% PFA by a BD Accuri C6 plus instrument. Antigen specific MFI values were summarized and depicted as columns.

FIG. 11B

Opsonophagocytic killing activity of the antigen-specific murine sera against P. acnes strains from different MLST types. Murine immune sera (n=5) raised against single protein Alhydrogel® formulations of P028 (PITP) and H4 and combination of both as well as adjuvant Alhydrogel® (PBS), as a negative control. Serial dilutions of murine immune sera were incubated with different P. acnes strains in the presence of human phagocytic cells. Opsonophagocytic killing titer (K50 value), defined as the highest serum dilution leading to more than 50% reduction of viable bacteria have been determined for P. acnes strains of genetic types IA1 (NCTC737), IA2 (P.acn31). IB (KPA171202), II (HL050PA2) and III (AsnI2). Reaction aliquots were plated onto blood agar plates at the beginning and at the end of the experiment. K50 values were ranked in reference to the K50 values of the negative control serum: K50 values between 500 and 2,500 were regarded as borderline (+/−), values above 2,500 up to 25,000 were marked positive (+), above 25,000 up to 300.000 highly positive (++), and above 300,000 extremely high positive (+++); K50 values below 500 or comparable to the adjuvant immunization alone were considered negative and below the detection limit (−).

FIG. 11C

Opsonophagocytic killing activity of the antigen-specific murine sera against P. acnes strains from different MLST types. Murine immune sera (n=5) raised against single protein Alhydrogel® formulations of P028-V7 (PITP) and H4-V3 and combination of both as well as adjuvant Alhydrogel® (PBS), as a negative control. Serial dilutions of murine immune sera were incubated with different P. acnes strains in the presence of human phagocytic cells. Opsonophagocytic killing titer (K50 value), defined as the highest serum dilution leading to more than 50% reduction of viable bacteria have been determined for P. acnes strains of genetic types IA1 (NCTC737), IA2 (P.acn31), IB (KPA171202), II (HL060PA1) and I1 (Asn12). Reaction aliquots were plated onto blood agar plates at the beginning and at the end of the experiment. K50 values were ranked in reference to the K50 values of the negative control serum: K50 values between 500 and 2,500 were regarded as borderline (+/−), values above 2,500 up to 25,000 were marked positive (+), above 25,000 up to 300,000 highly positive (++), and above 300,000 extremely high positive (+++); K50 values below 500 or comparable to the adjuvant immunization alone were considered negative and below the detection limit (−).

FIG. 12A

Alignment of P022 (DsA1), P027 (DsA2) and hybrid construct H4: the sequence section used from PROT-27 (DsA2) is underlined. Sequence components of P022 and P027 contained within the H4 hybrid molecule are indicated as shown in the FIGS. 12B and 12C respectively (based on the amino acid numbering of DsA1): N-terminal swapping region (“NSR”) (green) from S29 to I48; the first conserved sub-domain (“CSD1”) (yellow) from I49 to L130; the first swapping region (“SR1”) (grey) from G131 to S147: the second conserved sub-domain (“CSD2”) (yellow) from A148 to L267; the second swapping region (“SR2”) (grey) from A268 to T277; the third conserved sub-domain (“CSD3”) (yellow) from A278 to K323; a Pro-hr repeat containing region (“PT repeat region”) (red) from P324 to T361; and a C-terminal region (“CTR”), often with an LPXTG motif close to the C-terminus (blue) from S362 to F405. The used ‘swap sites’, i.e. the positions where PROT-22 (DsA1) and PROT-27 (DsA2) sequence regions were switched (as in a protein-cross-over) were chosen within swap regions, areas of least likelihood to interfere with structural elements of the protein, specifically alpha helices. The rationale and location of swap regions is also depicted in FIG. 7.

FIG. 12B

Full sequence alignment of selected, representative DsA1 sequences from various strains according to the entries in the public database. N-terminal swapping region (“NSR”) (green) from S29 to I48; the first conserved sub-domain (“CSD1”) (yellow) from I49 to L130; the first swapping region (“SR1”) (grey) from G131 to S147; the second conserved sub-domain (“CSD2”) (yellow) from A148 to L267; the second swapping region (“SR2”) (grey) from A268 to T277; the third conserved sub-domain (“CSD3”) (yellow) from A278 to K323; a Pro-Thr repeat containing region (“PT repeat region”) (red) from P324 to T361; and a C-terminal region (“CTR”), often with an LPXTG motif close to the C-terminus (blue) from S362 to F405.

FIG. 12C

Full sequence alignment of selected, representative DsA2 sequences from various strains. NSR (green) from A72 to K92; CSD1 (yellow) from 193 to L174; SR1 (grey) from S175 to S191; CSD2 (yellow) from A192 to L311; SR2 (grey) from A312 to T321; CSD3 (yellow) from A322 to E366; the PT repeat region (red) from P367 to T420, and CTR (blue) from H421 to A463.

FIG. 12D

Full sequence alignment of selected, representative PITP sequences from various strains. A signal peptide (“SP”) (blue) from M1 to A31; the extended neocarzinostatin family domain (“ENFD”) (green) from A32 to R164; a first swapping region (“SR1”) (grey) from E165 to K237; a heme-binding domain (“HbD”) from V238 to L396 (yellow), a second swapping region (“SR2”), including C-terminal LPXT(G) motif (“SR2”) (grey) from S397 to T430; and a hydrophobic C-terminal region (“hLAR”) (red) from G431 to 1467.

FIG. 13A

Correlation of surface binding of the human antibodies against P. acnes and the resulting opsonophagocytic killing (OPK) activity against P. acnes strains of genetic types IAI (NCTC737). Human serum samples were analyzed for their reactivity against P. acnes strains by a flow cytometry-based surface binding assay. Bacteria were cultivated in the presence of iron-chelator deferoxamine, re-suspended in HBSS buffer containing 2% BSA and incubated with 1:50,000 diluted human sera. The bound antibodies were detected with an Alexa Fluor 488 conjugated goat anti human Ig′ F(ab′)2 fragment. Median fluorescent intensities (MFI) were measured after cross-linking the surface bound antibodies with 2% PFA on a BD Accuri C6 plus instrument. The same serum samples were used to assess their opsonophagocytic killing titer (K50 titer), defined as the highest serum dilution leading to more than 50% reduction of viable bacteria. HL60 cells were differentiated to phagocytes and incubated with target bacteria and the serially diluted serum samples. Reaction aliquots were plated onto blood agar plates at the beginning and at the end of the experiment. Each serum sample was plotted according to its surface binding median fluorescence intensity (MFI, y-axis) and its opsonophagocytic activity titer (K50 value, x-axis).

FIG. 13B

The Graph shows data from the same experiment as in FIG. 13A for P. acnes strain IB (KPA171202). For strain IB human sera were diluted 1:25,000 in the surface binding assay, because of the lower signal intensities.

FIG. 14A

P. acnes phylotypes identified in the material isolated from the inflamed acne lesions (pustules) which were identified on the facial skin of the individuals with moderate to severe acne (IGA >2). Subject identification numbers are indicated with a CR prefix (study identification), followed by the subject identification number. DNA was extracted directly from the sample, subjected to PCR using SLST-specific primers and the phylotype was determined according to the published method and its associated public database (Scholz et al. 2014). All assigned amplicon reads have been noted.

FIG. 14B

Comparison of P. acnes phylotypes identified in the material isolated from inflamed acne lesions (pustules) and from the upper part of the skin pores of individuals with moderate to severe acne (IGA >2). Skin pores at two different locations were sampled on the face of each individual: forehead and cheek area. The SLST phylotypes identified in the skin pore material which was collected from the same facial skin area in which the pustule was identified and sampled, are shown in comparison to the SLST phylotypes identified in the acne lesion. The MLST phylotypes corresponding to the SLST phylotypes identified in each sample are shown in the adjacent column on the right. Subject identification numbers are indicated with a CR prefix (study identification), followed by the subject identification number. To increase DNA yield of skin pore samples, the material was propagated on the plates for 4 days at +37C in the absence of oxygen, or 3 days in the presence of oxygen at room temperature. The plates were scraped upon incubation, DNA was extracted from the total sample, subjected to PCR using SLST-specific primers and the phylotype was determined according to the published method and its associated public database (Scholz et al. 2014). Strains with more than 1000 amplicon reads are displayed in this list.

FIG. 15

ELISA detection of human antibody against hybrid H4-V1 (H4) antigen compared to full length proteins P022-V3 (DsA1), P027-V4 (DsA2) and P028-V1 (PITP) by in the serum samples from the individuals with moderate to severe acne (IGA >2). Maxisorb Immuno-plates were coated with P022-V3 (DsA1) and P027-V4 (DsA2) P028-V1 (PITP) and hybrid H4-V1, then blocked with BSA, incubated with serially diluted serum samples and detected using TMB and a peroxidase conjugated goat anti-human antibody. OD was measured at 450/620 nm and the IgG concentration in each sample expressed in micrograms per milliliter (μg/ml) based on the ELISA assay quantitative calibration curve. The error bars represent the SEM.

FIG. 16 A

Surface binding of murine immune sera (n=5, pooled) raised against Alhydrogel® formulations of single protein formulations of P028-V1 (PITP), P022-V3 (DsA1), H4V1, P027-V4 (Dsa2), P071 or adjuvant Alhydrogel® (PBS), as a negative control. Different P. acnes strains of Type IA1, IC and II were tested. Bacteria were cultivated in the presence of the iron-chelator deferoxamine. Cultures of different P. acnes strains were re-suspended in HBSS buffer containing 2% BSA and incubated with 1:1000 diluted immune sera. The bound antibodies were detected with an Alexa Fluor 488-conjugated goat anti-mouse IgG F′ab′)2 fragment. Median fluorescent intensity (MFI) values were measured after cross-linking with 2% PFA on a BD Accuri C6 plus instrument. The strain-specific MFI values are listed.

FIG. 16B

Graphic representation of the data from FIG. 16A showing the comparison of antigens based on the MFI values of the murine sera against P. acnes strains from the MLST type IA1 that express more DsA2 surface accessible epitopes compared to DsA1. MFI values of different strains are summarized per antigen serum and displayed as total MFI (stacked columns).

FIG. 16C

Graphic representation of the data from FIG. 16A showing the comparison of antigens based on the MFI values of the murine sera against P. acnes strains from the MLST type IA1 that express more DsA1 surface accessible epitopes compared to DsA2. MFI values of different strains are summarized per antigen serum and displayed as total MFI (stacked columns).

FIG. 16D

Graphic representation of the data from FIG. 16A showing the comparison of antigens based on the MFI values of the murine sera against P. acnes strains from the MLST type IC that express more DsA1 surface accessible epitopes compared to DsA2. MFI values of different strains are summarized per antigen serum and displayed as total MFI (stacked columns).

FIG. 16E

Graphic representation of the data from FIG. 16A showing the comparison of antigens based on the MFI values of the murine sera against P. acnes strains from the MLST type II that express more DsA2 surface accessible epitopes compared to DsA1. MFI values of different strains are summarized per antigen serum and displayed as total MFI (stacked columns).

FIG. 16F

Graphic representation of the data from FIG. 16A showing the comparison of antigens based on the MFI values of the murine sera against P. acnes strains from the MLST type IA1. MFI values of different strains are summarized per antigen serum and displayed as total MFI (stacked columns).

FIG. 17A

Schematic visualization of the P022 (Dsa1) domains and scheme of fragment constructs and derivatives of the protein that have been designed and expressed. The scheme has been created based on Table IA and IB sequence information and the Uniprot reference protein Q6A5X9. Signal peptide (“SP”) region that can be present from M1 to A28; N-terminal swapping region (“NSR”) from S29 to I48: the first conserved sub-domain (“CSD1”) from 149 to L130; the first swapping region (“SR1”) from G131 to S147; the second conserved sub-domain (“CSD2”) from A148 to L267; the second swapping region (“SR2”) from A268 to T277; the third conserved sub-domain (“CSD3”) from A278 to K323; a Pro-Thr repeat containing region (“PT repeat”) from P324 to T361; and a C-terminal region (“CTR”), often with an LPXTG motif close to the C-terminus from S362 to F405. All of the fragments in the graph have N-terminal 6×His-tags. For the derivatives the 6×His tag position is indicated with a black star and the Avi-tag by a hexagon and the Avi-tag position by a hexagon.

FIG. 17B

Schematic visualization of the P027 (Dsa2) domains and scheme of fragment constructs and derivatives of the protein that have been designed and expressed. The scheme has been created based on Table 1A and 1B sequence information and the Uniprot reference protein Q6A5P9. Signal peptide (“SP”) region that can be present from M1 to A71; NSR from A72 to K92; CSD1 from 193 to L174; SR1 from S175 to S191; CSD2 from A192 to L311; SR2 from A312 to T321; CSD3 from A322 to E366; the PT repeat region from P367 to T420, and CTR from H421 to A463. The 6×His tag position is indicated with a black star and the Avi-tag by a hexagon.

FIG. 17C

Schematic visualization of the P022 (Dsa1) and P027 (Dsa2) domains as described in FIGS. 17A and 17B, respectively. The Hybrids of the P022 and P027 proteins domains are visualized through combination and merging of the domains of Dsa1 and Dsa2, amino acid exchanges within the H4 sequence are indicated in the description of the hybrid. The 6×His tag position is indicated with a black star and the Avi-tag by a hexagon. Sequence information can be found in Table 1B.

FIG. 18A

Binding of murine serum antibodies generated by antigen immunizations to the cell surface of P. acnes strains from the representative genetic types. Murine immune sera (n=5) raised against Alhydrogel® formulations of single protein formulations of P022-V2 (DsA1), fragments of P022 (F4, F5, F7, F8, F9, F11-F14), P027-V3 (DsA2), a fragment of P027 (P027F1), hybrid constructs (H2-H5, H4-V1) or adjuvant Alhydrogel® (PBS), as a negative control, were analysed in a flow cytometry based bacterial surface binding assay. Cultures of genetic types IA1 (NCTC737), IA2 (P.acn31). IB (KPA171202), IC (PV66), II (HL050PA2) and III (Asn12) were re-suspended in HBSS buffer containing 2% BSA and incubated with 1:1000 diluted immune sera. Antibody binding was detected with an Alexa Fluor 488-conjugated goat anti mouse IgG (F′ab′)2 fragment. Median fluorescent intensities (MFI) were measured after cross-linking with 2% PFA by a BD Accuri C6 plus instrument. Individual strain-specific MFI values are grouped and depicted as columns for each immunization antigen.

FIG. 18B

Comparison of the murine immune sera raised against DsA1 and DsA2 fragments and hybrids determined in the opsonophagocytic killing assay against P. acnes strain NCTC737 (MLST type IA1). Murine immune sera (n=5) were raised against single protein Alhydrogel® formulations of P027-V3 (Dsa2), P022-V2 (Dsa1), P022 fragments (F4, F7, F8, F9, F14, F11). P027 fragment (P027F1) and the hybrid constructs (H2-H5). Serial dilutions of murine immune sera (starting at 1:10.000 dilution) were incubated with P. acnes strain NCTC737 and human phagocytic cells. Reaction aliquots were plated onto blood agar plates at the beginning and at the end of the experiment. Percentage of the reduction of viable P. acnes colony forming units (cfu) compared at each serial dilution to the corresponding dilution of the murine serum induced against the adjuvant Alhydrogel® (PBS) is shown as percentage of the killing: % killing (ctr).

FIG. 18C

Graphic representation of the data from FIG. 16B showing the comparison of antigens based on the percentage of opsonophagocytic killing at each respective serial serum dilution in comparison to the same dilution of the murine serum induced against the adjuvant Alhydrogel® (PBS). DsA1 fragment F4 had not induced phagocytic killing at any dilution and its values align along the X-axis.

FIG. 19A

Semi-quantitative surface plasmon resonance (Biacore) analysis of polyclonal mouse serum pools. Binding signals of antibodies raised against Alhydrogel® formulations of P022-V2 (DsA1), fragments of P022 (F4, F5, F7, F8, F9, F11-14), P027-V3 (DsA2), a fragment of P027 (P027F1), hybrid constructs (H2-H5, H4-V1) or adjuvant Alhydrogel® (PBS), as a negative control, to immobilized P022-V4, P027-V5 and H4-V4 are shown. Binding of diluted serum samples to proteins individually immobilized in consecutive flow cells at the end of sample injection is depicted in response units (RU). High values indicate higher concentrations of protein-specific antibodies. The binding signal correlates with the amount of bound antibodies to the antigen immobilized on the cell surface. The more mass (antibodies) is bound on the chip surface the higher the response units (RU).

FIG. 19B

Qualitative surface plasmon resonance (Biacore) analysis of polyclonal mouse serum pools. Binding characterization of antibodies raised against Alhydrogel® formulations of single protein formulations of P022-V2 (DsA1), fragments of P022 (F4, F5, F7, F8, F9, F11-14), P027-V3 (DsA2), a fragment of P027 (P027F1), hybrid constructs (H2-H5, H4-V1) or adjuvant Alhydrogel® (PBS), as a negative control, to immobilized P022-V4, P027-V5 and H4-V4 was examined. Relative off-rate analysis evaluated during 300s after end of sample injection to proteins individually immobilized in consecutive flow cells is shown. Low values (1/s) indicate slow dissociation rates (high affinity): higher values indicate fast dissociation from the proteins immobilized on the chip surface (low affinity).

FIG. 20A

Schematic visualization of the P028 (PIPT) domains and scheme of P028-V7 fragment constructs that have been designed and expressed. The table has been created based on Table IC sequence information and the Uniprot reference sequence Q6A9N1. Signal peptide from M1 to A31; ENFD from A32 to R164; SR1 from E165 to K237. HbD from V238 to L396, SR2 (including C-terminal LPXT(G) motif (i.e. including LPXT, but not the G) from S397 to T430, and hLAR from G431 to 1467. All the fragments have 6×His-tags (F1 and F2 at C-terminus, all other fragments at N-terminus) and carry the P028-V7 specific Cystein amino acid exchanges (C201S: C372S).

FIG. 20B

Schematic visualization of the P028-V7 (PIPT) and H4 hybrid constructs that have been designed and expressed. The P028-V7 Fragments have been fused to the C-terminus of H4-V3, after a short PT repeat linker region, to increase solubility of the protein fragments and enable the expression of P028 protein fragments that are shorter than 140aa. The scheme has been created based on Table 1C sequence information. All of the hybrids have an N-terminal 6×His-tags and carry the P028-V7 specific Cystein amino acid exchanges (C20 IS; C372S).

FIG. 20C

Schematic visualization of the P028 (PIPT) domains and P028-V7 amino acid exchange constructs that have been designed and some of them also expressed (M1 and M7). The table has been created based on Table 1 A sequence information.

FIG. 21A

Binding of murine serum antibodies generated by antigen immunizations to the cell surface of P. acnes strains from the representative genetic types. Murine immune sera (n=5-10) raised against Alhydrogel® formulations of single protein formulations of P028 (PITP), derivatives of P028 (P028-VI, P028-V2, P028-V7), fragments of P028-V7 (F1, F2. F7, F16, F18-F20) and adjuvant Alhydrogel® (PBS), as a negative control, were analysed in a flow cytometry based bacterial surface binding assay. Cultures of genetic types IA1 (NCTC737), IA2 (P.acn31), IB (KPA171202). IC (PV66), II (HL060PA1) and III (Asn12) were re-suspended in HBSS buffer containing 2% BSA and incubated with 1:1000 diluted immune sera. Antibody binding was detected with an Alexa Fluor 488-conjugated goat anti mouse IgG (F′ab′)2 fragment. Median fluorescent intensities (MFI) were measured after cross-linking with 2% PFA by a BD Accuri C6 plus instrument. Individual strain-specific MFI values are grouped and depicted as columns for each immunization antigen.

FIG. 21B

Graphic representation of the data from FIG. 16A showing the comparison of antigens based on the MFI values of the murine sera against P. acnes strains from the MLST type type IB (KPA171202) and III (Asn12). MFI values of different strains are summarized per antigen serum and displayed as total MFI (stacked columns).

FIG. 21C

Binding of murine serum antibodies generated by antigen immunizations to the cell surface of P. acnes strains from the representative genetic types IA1 (NCTC737), IA2 (P.acn31), IC (PV66) and II (HL60PA1). Murine immune sera (n=5-10) raised against Alhydrogel® formulations of single protein formulations of P028-V7, H4-V3, fusion constructs thereof (C12, C13) and P028 amino acid exchange derivatives (M1, M7) as well as adjuvant Alhydrogel® (PBS), as a negative control, were analysed in a flow cytometry based bacterial surface binding assay as explained in FIG. 21A. Individual strain-specific MFI values are grouped and depicted as columns for each immunization antigen.

FIG. 21D

Binding of murine serum antibodies generated by antigen immunizations to the cell surface of P. acnes strains from the genetic types IB (KPA171202) and III (Asn12). Murine immune sera (n=5-10) raised against Alhydrogel® formulations of single protein formulations of P028-V7, H4-V3 fusion constructs thereof (C12 C13) and P028 derivatives (M1, M7) as well as adjuvant Alhydrogel® (PBS), as a negative control, were analysed in a flow cytometry based bacterial surface binding assay as described in FIG. 21A. Individual strain-specific MFI values are grouped and depicted as columns for each immunization antigen.

FIG. 22A

Opsonophagocytic killing activity of the antigen-specific murine sera against P. acnes strains from different MLST types. Murine immune sera pools (n=5) raised against single protein Alhydrogel® formulations of single protein formulations of P028-V7 (PITP), derivatives of P028 (M1, M7), fragments of P028-V7 (F1, F2, F7, F16, F18-F20), fusion constructs with H4-V3 (C12, C13) and adjuvant Alhydrogel® (PBS), as a negative control were tested and compared to negative assay controls. Serial dilutions of murine immune sera were incubated with different P. acnes strains in the presence of human phagocytic cells. Opsonophagocytic killing titer (K50 value), defined as the highest serum dilution leading to more than 50% reduction of viable bacteria have been determined for P. acnes strains of genetic types IA1 (NCTC737), IA2 (P.acn3l), IB (KPA171202), IC (PV66), II (HL060PA1) and III (Asn12). Reaction aliquots were plated onto blood agar plates at the beginning and at the end of the experiment. K50 values were ranked in reference to the K50 values of the negative control serum: K50 values between 500 and 2,500 were regarded as borderline (+/−), values above 2,500 up to 25,000 were marked positive (+), above 25,000 up to 300,000 highly positive (++), and above 300,000 extremely high positive (+++); K50 values below 500 or comparable to the adjuvant immunization alone were considered negative and below the detection limit (−).

FIG. 22B

Graphic representation of the data from FIG. 22A showing the comparison of antigens based on the K50 titer values of the murine sera against P. acnes strains from the six representative MLST types IA1 (NCTC737), IA2 (P.acn31), IB (KPA171202), IC (PV66), II (HL060PA1) and III (Asn12). K50 titers of different strains are summarized per antigen serum and displayed as total K50 titers (stacked columns).

FIG. 22C

Graphic representation of the data from FIG. 22A showing the comparison of antigens (P028 fragments and P028-V7) based on the K50 titer values of the murine sera against P. acnes strains from MLST types IA1 (NCTC737), IA2 (P.acn31). IC (PV66) and II (HL60PA1). K50 titers of different strains are summarized per antigen serum and displayed as total K50 titers (stacked columns).

FIG. 22D

Graphic representation of the data from FIG. 22A showing the comparison of antigens (P028 fragments and P028-V7) based on the K50 titer values of the murine sera against P. acnes strains from MLST types IB (KPA171202) and III (Asn12). K50 titers of different strains are summarized per antigen serum and displayed as total K50 titers (stacked columns).

FIG. 23A

Graphic representation of the median EC50 values obtained from five murine sera raised against different antigens evaluated in ELISA against the respective immunizing antigen. Serial dilutions of murine immune sera (n=10) raised by immunization with Alhydrogel® formulations of P028-V7 (PITP), fragments of P028-V7 (F1, F2, F7, F16, F18-F20), derivatives of P028-V7 (M1, M7) and fusion constructs with H4-V3 (C12, C13) as well as an adjuvant Alhydrogel® (PBS) negative control were tested to measure the amount of binding to the respective protein antigen used in the immunization (the immunogen) to determine the antibody EC50 titers after 4 immunizations. Error bars indicate the variability of the EC50 values within the study group.

FIG. 23B

Graphic representation of the EC50 values obtained from the five pooled animal sera raised against different antigens evaluated in ELISA against the P028-V7 (PITP). Serum antibodies induced by immunization with protein P028-V7 (PITP), fragments of P028-V7 (F1, F2, F7, F16, F18-F20), derivatives of P028-V7 (M1, M7) and fusion constructs with H4-V3 (C12, C13) were investigated by ELISA. Serial dilutions of murine immune sera pools (n=10) raised by immunization with Alhydrogel® formulations of P028-V7 (PITP), fragments of P028-V7 (F1, F2, F7, F16, F18-F20), derivatives of P028-V7 (M1, M7) and fusion constructs with H4-V3 (C12, C13) as well as an adjuvant Alhydrogel® (PBS) negative control were tested to measure the amount of binding to the P028-V7 (PITP) to determine the antibody EC50 titers after four immunization rounds. For the pre-immune sera of the immunized mice no EC50 values could be determined because of no or low reactivity.

FIG. 23C

EC50 values of the serum samples tested as explained in the FIGS. 23A and 23B, shown in comparison. Serum antibodies induced by immunization with protein P028-V7 (PITP), fragments of P028-V7 (F1, F2, F7, F16, F18-F20), derivatives of P028-V7 (M1, M7) and fusion constructs with H4-V3 (C12, C13) were investigated by ELISA. Serial dilutions of murine immune sera pools (n=10 for coating antigen P028-V7) or means of murine immune sera (n=10 for self-antigen coating) raised by immunization with Alhydrogel® formulations of P028-V7 (PITP), fragments of P028-V7 (F1, F2, F7, F16, F18-F20), derivatives of P028-V7 (M1, M7) and fusion constructs with H4-V3 (C12, C13) as well as an adjuvant Alhydrogel® (PBS) negative control were tested to measure the amount of binding to the P028-V7 or the respective protein antigen (immunogen) to determine the antibody EC50 titers after 4 immunization rounds. Indication of the adjuvant Alhydrogel® (PBS) negative control is not applicable (n/a) for the self-antigen measurement, since for each antigen a separate value would have to be assigned. No EC50 titer for the PBS control on different immunization antigens could be assigned because the reactivity was below detection level.

FIG. 24A

Model of the expected vaccine mode of action in protection of hair follicles. Vaccination is expected to increase the amount and quality of P. acnes-specific antibodies which are both, locally secreted and which diffuse from the blood capillaries into the hair follicles. The increased level of functionally active (immunorelevant) antibodies can strengthen local immune defenses and make them more efficient in counteracting increased proliferation and virulence. In the cases where P. acnes is able to resist or overcome the homeostatic control of the bacterial proliferation by the skin innate immune defenses, neutrophils are additionally recruited from the surrounding blood capillaries to help stop and clear the infection and prevent its spreading to the surrounding skin tissue. In this case, the increased levels of the immunorelevant antibodies also increase the killing efficiency of the neutrophils and lead to resolution of the inflammation, before the more visible clinical symptoms are visible on the skin surface. Moreover, in the case of the pre-existing lesions, increased level of high quality antibodies may also help speed up the recovery.

FIG. 24B

Model of additional benefits of the P. acnes vaccine in improved regulation of immune cell function and turnover in the course of an inflammatory response. An augmented inflammatory response is also potentiated by the uncontrolled release of the intracellular contents of the human cells that participate in skin immune defense and which can die by the process of apoptosis or necrosis. In cases where P. acnes is able to resist or overcome the homeostatic control of the bacterial proliferation by the skin innate immune defenses, the inflammation persists not only due to the increased activity of P. acnes, but also due to the additional burden created by the uncontrolled release of the contents from the necrotic phagocytic cells. On the contrary, when sufficient levels of opsonizing antibodies are present, phagocytes can more efficiently capture and digest bacterium and either leave the site of infection or die by apoptosis, which is a tightly regulated process that occurs under physiological conditions.

### 7 EXAMPLES

The foregoing description will be more fully understood with reference to the following examples. Such examples are, however, merely representative of methods of practicing one or more embodiments of the present invention and should not be read as limiting the scope of invention.

### Example 1: P. acnes Vaccine Antigen Evaluation

### Recombinant Expression of Selected Proteins

A large number of P. acnes proteins were expressed and purified using E. coli strain BL21 (DE3) upon plasmid-mediated over-expression of the corresponding gene, as indicated in Table 1 (A and B). Proteins containing a His-tag were purified via a Ni-NTA column, proteins without His-tag have been purified via sequential ion-exchange and size-exclusion columns. Proteins were soluble in PBS or similar buffer systems and were stored as lyophilized products at −80° C. Expressed proteins had a purity of >90% according to CoA and were used for animal immunizations.

The summary of the sequences used for designing different constructs and the sequence strain origin are presented in the Table 1. If necessary, for the purpose of expression, some protein sequences were optimized by removing short stretches of hydrophobic amino acids or by single site mutations; details are shown in the Table 1.

### Vaccine Formulation and Desorption

Proteins were formulated on Alhydrogel (InvivoGen, vac-alu-250) at a concentration of 100 μg/ml by overnight incubation at 4° C.

For desorption of formulated proteins, formulations were centrifuged at 4000 rpm for 5 minutes and the supernatants were kept for subsequent analysis. The pellet was resuspended in 0.5 M sodium phosphate buffer (Sigma, S3264) containing 1% zwittergent 3-14 (Sigma-Aldrich, 17763) and incubated at RT for 30 minutes. After incubation, 2× Laemmli buffer containing 10 mM DTT (Sigma-Aldrich, 646563) was added and samples were incubated at 99° C. for 10 minutes. After cooling down to RT, 750 mM iodoacetamide (Sigma-Aldrich, 16125) was added and the samples were incubated overnight at 4° C. with gentle agitation. The following day, the samples were centrifuged at 4000 rpm for 5 minutes and the supernatant was collected as desorbed fraction.

### Immunization of Animals

Mice (BALB/C) were immunized five times within three months with the respective protein in combination with an adjuvant. Mice have been immunized with 10 μg of Alhydrogel® coupled Antigen per dose, in case of combination of more than one antigen, 10 μg per antigen was used. Rabbits have been immunized four times with 50 μg of oil-in water adjuvanted protein per dose.

The increase in antibody levels during the immunizations was monitored using ELISA to quantitate the amount of antigen-specific antibodies.

This total antigen-specific IgG ELISA is used for the quantitative detection of antibodies raised against specific P. acnes antigens. 96-well microtiter plates (Nunc-Immuno, Maxisorb) are coated for 18±2 h at 4° C. with 70 μl of recombinant protein of interest (concentration 0.5-2 μg/ml in PBS, depending on the coating protein). After washing each plate with Wash Buffer (PBS supplemented with 0.1% Tween-20), 100 μl of Blocking Buffer (PBS with 2% BSA and 0.1% Tween-20) were added and incubated for a minimum of 30 min on an orbital shaker to prevent unspecific binding. For the determination of antigen-specific antibody titers, 50 μl of a 5-fold dilution series of heat-inactivated (45 min 56° C.) rabbit or mouse serum was applied to the coated and blocked plate and incubated for 1 hour on an orbital shaker. The primary antibody solution was then removed followed by washing steps and subsequent incubation with 50p of the 1/20,000 diluted horse radish peroxidase (HRP)-linked secondary antibody (Peroxidase AffiniPure Goat Anti-Rabbit IgG, Fc fragment specific, Jackson Immuno Research; Peroxidase AffiniPure Goat Anti-Mouse IgG, Fc fragment specific, Jackson Immuno Research). After washing the plates, HRP activity was detected by adding 100 μl of tetramethylbenzidine substrate (TMB, Thermo Fisher) for 10 min incubation, yielding a blue color (Amax=370 nm and 652 nm) that changes to yellow (Amax=450 nm) upon addition of 50 μl of 3N sulphuric acid (Sigma) stop solution. The reaction was quantified using a microtiter plate photometer (Tecan Sunrise). Background values were determined by reading reactions that lacked the primary antibody. The amount of IgG for each test sample was measured in a dilution series in duplicates and the EC50 value was calculated from the sigmoidal curve fit of these measurements by using GraphPad software.

Immunization of mice and rabbits with P. acnes vaccine antigen candidates led to a substantial increase in the amount of antigen-specific antibodies, with the end-titers or EC50 values determined by ELISA.

All antigens demonstrated immunogenicity and were capable of inducing antigen-specific immune response in the murine immunization studies according to the present invention, as evidenced by their ability to induce antigen-specific antibodies detectable in ELISA with EC50 titer of at least 10-fold higher compared to the immunization with adjuvant in the absence of the protein antigen (PBS) and even higher than 10 fold-increase compared to the corresponding pre-immune serum samples (FIG. 2)

### Bacterial Strains and Growth Conditions

A total of 110 P. acnes isolates have been obtained from various commercial and academic sources, or isolated from humans in the course of Origimm's clinical research studies (please refer to the Table 2). The strains were comparatively analyzed in this study, handled and grown under the same conditions. All strains were categorized into six phylotypes IA1, IA2, IB, IC, II and III by multiplex touchdown PCR for rapid typing (Barnard et al. 2015) or classified into specific ribotypes according to the scheme of Fitz-Gibbon et al., 2013 (Fitz-Gibbon et al. 2013). Unless stated otherwise, all strains were incubated at 37° C. anaerobically using Gas Pak™ EZ Anaerobic Container system system (BD). For use in the experiments, the respective isolates were streaked from −80° C. stocks onto Brucella blood agar plates with Hemin and Vit K (BD™, Cat. Nr. 25509), incubated for 3-5 days, and kept on 2-8° C. for a maximum of 14 days. For liquid cultures bacterial colonies from the plate were resuspended in Thioglycolate broth (BD™, Cat. Nr. 221788) and incubated to an appropriate optical density and used as inoculum for the individual experiments. To mimic iron starvation conditions, pre-cultures have been diluted to an OD600 of 0.1 and supplemented with a final concentration of 500 μM Deferoxamine mesylate (DMF, Sigma) followed by an incubation under standard P. acnes growth conditions for 15-20 hours.

The present data demonstrated that under iron limiting conditions, the expression of PITP was significantly increased, whereas the expression of all other surface proteins remained unchanged under the same conditions, and the expression of another Hta-like iron-binding protein and suggested immunologically relevant P. acnes surface antigen. P071 (PA-4687; Lodes. 2006) was only slightly increased on some strains (FIG. 3). Since the iron limiting conditions did not change the expression patterns of any other antigen that was studied, the iron-limiting conditions were applied in the subsequent surface-binding assays in order to capture the iron-binding proteins on the cell surface under the same iron depleting condition.

P. acnes Surface Binding

Mid-exponential P. acnes growing cultures were pelleted by centrifugation at 1000×g for 4 min and washed twice with ice-cold Hanks buffered salt solution (HBSS, Gibco) supplemented with 0.5% bovine serum albumin (BSA, H2B). The final pellet was resuspended in HBSS in the presence of blocking agent (2% BSA) to give a cell density of 4×10⁶ cells/ml. Bacteria (2×10⁵ cfu per well) were incubated with heat-inactivated (45 min 56° C.) murine or rabbit sera in a final dilution of 1/1000 in HBSS/2% BSA for 30 min. The initial incubation was performed in a volume of 100 μl in a 96 well microtiter plate. After centrifugation at 1000×g, bacterial pellets were washed with HBSS/0.5% BSA, and resuspended in 100 μl HBSS/0.5% BSA including the detecting antibody (depending on the primary antibody from mouse or rabbit, respectively; Alexa Fluor 48′ F(ab′)2-Goat anti-Mouse IgG (H+L) Cross-Adsorbed Secondary Antibody, Thermo Fisher, Cat. Nr. A11017; Alexa Fluor 48′ F(ab′)2 fragment of goat anti rabbit IgG, Thermo Fisher, Cat. Nr. A11070) diluted 1/500 in HBSS/0.5% BSA and incubated with the cells for 30 min. Thereafter, 50 μl of Syto60 (Thermo Fisher, Cat. Nr. S11342) diluted 1/500 in HBSS was added to the reaction, followed by a 15 min incubation, to aid visualization of bacterial cells and facilitate flow cytometry analysis. Before the analysis, bacteria were spun down, washed in HBSS and incubated in 2% Paraformaldehyde (PFA, Alfa Aesar) for 10 min. 2% PFA was removed by centrifugation and the fixed bacteria were resuspended in HBSS. The fluorescence intensities which correlated with the amount of anti-P. acnes antibodies bound to the surface of P. acnes, were measured using a BD Accuri C6 plus flow cytometer equipped with a 96-well plate loader. Data Analysis has been performed using FLOWJO® software and median fluorescent intensities of the detecting antibody have been calculated.

All sera induced after immunization with respective antigens were tested using the surface binding assay. Although all antigens induced antigen-specific antibodies in the murine studies as demonstrated in ELISA (FIG. 2), a large number of these antigens did not induce antibodies that were able to bind to the surface of genetically different P. acnes strains. Interestingly, only antibodies induced by P022 (DsA1), P027 (DsA2) and P028 (PITP) consistently bound P. acnes cell surface of the strains from different P. acnes phylotypes. This is surprising and in contrast to other prior art proteins, which were also suggested to be important virulence factors, immunogenic and exposed on the cellular surface and/or secreted (FIG. 1A; P002, P005, P035, P042, P046, P068, P069, P070, P071). Indeed, none of these prior art antigens were detected by the present flow cytometry-based surface binding assay on representative strains of all 6 MLST phylotypes or the surface binding of antibody was extremely low (FIG. 1A). The expression and accessibility of P022 (DsA1) and P027 (DsA2) on the cell surface of live P. acnes was not only significant on the cell surface of the phylotype IA1, but also on the phylotypes IA2, II and IC (FIG. 1A). PITP was detected in variable amounts on the surface of all six MLST phylotypes, including also Type IB and III (FIG. 1A), which were negative for P022 (DsA1) and P027 (DsA2).

The sera induced after immunization with P022-V3 (DsA1), P027-V4 (DsA2) and P028-VI (PITP) were further tested for surface binding on 60 different strains which were tested and grouped according to the 16S ribotype classification scheme (FIG. 4). The data obtained with the present invention demonstrate that the surface accessibility of the selected vaccine candidates is significantly lower on the ribotypes which are not commonly isolated from acne-prone skin (RT2 and RT6), and tends to be higher on those strains that have been implicated in acne pathogenesis (RT4, RT5, RT8): however exceptions were detected in the course of the present invention in which a completely different surface expression pattern was detected on the strains that are classified under the same ribotype. For example, in the surface binding analysis of antibodies induced by immunization with P022 (DsA1). P027 (DsA2) and P028 (PITP), the strains marked in FIG. 4 with the number 45, 79 and 87 are according to the prior art classified under RT1, and the strains 29 and 80 are both classified as RT5 (Table 2), but show significantly different expression of the three virulence factors on the surface of these P. acnes strains (FIG. 4). Variations in the surface antigen expression and detection were also noted on the strains classified under RT2: antibodies raised against DsA1, DsA2 and P028 had recognized and bound these three virulence factors much more strongly on the strain 102 compared to the strains 100 and 105.

### Opsonophagocytic Killing (OPK) Assay

HL60, a human promyelocytic leukemia cell line (ECACC) was maintained in RPMI medium 1640 1×+Glutamax (Gibco) supplemented with 10% heat inactivated Fetal Bovine Serum (FBS, Gibco). Cells were grown and differentiated to phagocytes in growth medium supplemented with 0.8% Dimethyl Formamide (DMF, Sigma) for 3 days. For the OPK assay P. acnes liquid cultures were grown to a mid-logarithmic growth phase, bacterial cells were washed twice in HBSS/0.5% BSA and diluted to about 1.5×10⁸ cells/ml in HBSS 2% BSA. The bacteria were then incubated with the 6.6×10⁶ cells/ml DMF-differentiated HL-60 cells at a MOI of 1:400 in RPMI1640+10% FCS+20 mM Glucose for at least 24 hours or longer at 37° C., in the presence of 5% CO2 in microtiter plates covered with gas permissive sealing foil (Aera seal). Assay reactions were prepared in duplicates. Serial dilutions of heat-inactivated serum samples were incubated in the presence of phagocytic cells with live P. acnes. Multiple reaction aliquots were plated before opsonization (T=0) and at the end of the incubation with phagocytes. After the incubation, 100 μl of a 1:64, 1:32 or 1:16 dilution (depending on the bacterial strain) of the reaction were plated onto Brucella blood agar plates supplemented with Hemin and vitamin K. After anaerobic incubation of the plates at 37° C. for a minimum of 70 hours, the colonies on the plates were counted with a Colony Counter (AES Laboratories). Median cfu/reaction values were derived from each reaction and the percentage of OPK activity was calculated as follows: the number of cfu after the incubation with phagocytes is compared to the cfu of the corresponding negative controls (the reaction containing all assay components except serum and/or the reaction containing corresponding pre-immune serum), and used in the following formula: 100-100×(CFUimmune/CFUcontrol). The opsonophagocytic killing activity of the human or antigen-specific hyperimmune sera containing anti-P. acnes antibodies was expressed as a K50 titer: this titer corresponds to the highest serum dilution showing more than 50% decrease in the viable bacterial counts (colony forming units, cfu) in comparison to a negative control (reaction without antibody added or a corresponding preimmune serum tested at the same dilution). The resulting K50 titer is a direct measure of the OPK activity of the serum raised against the evaluated protein antigen. This is a clear indication of an antigen's potency to induce the generation of bactericidal anti-P. acnes antibodies upon immunization or the potency of the antibodies generated in human host against the native protein antigens expressed by P. acnes as the consequence of its interaction with the human immune system.

All sera induced after immunization with antigens were tested using the OPK assay. The significance of the surface-binding of antibodies induced by immunization with P022 (DsA1), P027 (DsA2) and P028 (PITP) (FIG. 1A) could be confirmed in the OPK assay which demonstrated that only the antibodies induced by immunization with the antigens P022 (DsA1). P027 (DsA2) and P028 (PITP) were able to induce significant opsonophagocytic killing of P. acnes, whereas the antibodies induced by the immunization using other prior art antigens were clearly inferior (FIGS. 1B, 1C and 1D). While the antibodies induced by immunization with P022 (DsA1) and P027 (DsA2) induced opsonophagocytic killing against MLST phylotypes IA1, IA2, IC and II, the antibodies induced by immunization with P028 (PITP) were in addition also able to induce opsonophagocytic killing of the bacterial strains of MLST phylotypes IB and to a lesser extent phylotype III (FIGS. 1B, 1C and 1D).

### Example 2: Combination of Antigens DsA1, DsA2 and PITP

Animals have been immunized with vaccines containing solely antigens P022 (DsA1), P027 (DsA2) or P028 (PITP) as well as with vaccines containing the combinations of these antigens according to the method of “Immunization of animals” as described in Example 1. The antibodies raised by this immunization have been tested in a surface binding assay (as described under Example 1).

The studies performed for the present invention in the surface binding assay showed that the best cross-reactive vaccine/cross-type-reactive vaccine, effects can be obtained by combining the antigens P028 (PITP), P022 (DsA1) and P027 (DsA2) (FIG. 5).

### Example 3: Analysis of Fragments of DsA1 and DsA2

### Analysis of Fragments of DsA1 and DsA2

A number of fragments of P022 (DsA1) and P027 (DsA2) and hybrid molecules of P022 and P027 have been produced (Table 1B, FIG. 17A, FIG. 17B, FIG. 17C) and used for animal immunizations (rabbits and mice) as described in Example 1.

Immunization with four overlapping fragments P022F1 to P022F4 of protein P022 (DsA1), which together comprise the full sequence of P022 (DsA1), had induced antibodies which had a weaker activity (FIG. 6) when compared to antibodies induced after immunization with the full-length protein.

Immunization with fragments which represent primarily the C-terminus (F4, F12 and F13) and the N-terminus (F10) of P022 (DsA1), induced antibodies which had a much lower surface binding ability compared to antibodies induced by immunization with the full length proteins (FIG. 6 for F4, FIG. 9 for F10, F12 and F13. FIG. 18A).

Immunization with fragments which represent more central parts of P022 (DsA1) (F11) and P027 (DsA2) (P027-F1) induced antibodies which had surface binding comparable to antibodies induced by immunization with the full length proteins (FIG. 9, FIG. 18A). This could also be confirmed in the OPK assay and in Biacore analysis (FIG. 18B, 18C, 19A, 19B). Especially P022F4 which contains mostly C-terminus and the PT repeats does not contribute to either surface binding or to the induction of the functional activity (OPK).

### Semi-Quantitative and Qualitative (Affinity) Characterization of Vaccine Induced ABs by Surface Plasmon Resonance (SPR/Biacore Analysis).

P022-V4 was immobilized on Fc2, P027-V5 on Fc3, H4-V4 on Fc4 and on Fc1 no protein was immobilized and was used as a reference flow cell. All four flow cells were blocked with free Biotin after the immobilization procedure. During the measurement of serum samples murine monoclonal antibodies specific for all three proteins were injected in regular intervals to control the chip performance and protein integrity. To characterize the binding level of antibodies raised against Alhydrogel® formulations of single protein formulations of P022-V2 (DsA1), fragments of P022 (F4, F5. F7, F8, F9, F11-14), P027-V3 (DsA2), a fragment of P027 (P027F1), hybrid constructs (H2-H5, H4-V1) or adjuvant Alhydrogel® (PBS), as a negative control two parameters were evaluated: Firstly, the binding at the end of injection (end of association phase) given in response units (RU) which is a semi-quantitative measure for the concentration of the protein-specific antibodies and secondly the relative off-rate of the bound antibodies which reflects the affinity of the polyclonal antibodies during the dissociation phase. Binding is reported in response units and relative off-rate was determined using a separate Kd fit with the 1:1 Langmuir dissociation fitting model and is reported in signal decrease per second (1/s).

### Example 4: Generation and Analysis of Hybrids of DsA1 and DsA2

### Protein Modelling and Swap Point Determination

After recognition of S. aureus extracellular matrix binding protein ebhA as a possible template for DsA1, based on fold-recognition an initial structure model was generated. In a second run 100 models and for each 15 loop-models (1500 in total) were automatically generated in-silico. Each of those represent a new attempt for obtaining a local (or optimally) global minimum. Among those, the ten best were selected based on the scoring energy function and subsequently secondary structure was derived for each residue using dssp software. For helix-bundle linkers (generally denoted ‘swap regions’) and the degree of obtained secondary structure was considered. This method was therefore used to simulate the preference for secondary structure elements, or rather the extent of conversion of elongated coils to structured helices. Swap regions were then defined as those not necessarily structured into helices, where a central core is never structured. These swap regions are therefore regions between structurally defined, presumably functional adhesin domains, where switching from DsA1 to DsA2 and back should not disrupt structural integrity (FIG. 7B).

This bioinformatics analysis revealed a 3-domain structure of the protein DsA1 (FIG. 7A) and DsA2 (FIG. 7C).

### Sequence Selection and Alignment

Amino-acid sequences were aligned using clustal omega multiple sequence alignment software version 1.2.4 obtained from EMBL-EBI. For the preparation of FIG. 12B protein sequences that are available in the public databases and which are derived from various P. acnes strains were manually selected.

Multiple sequence alignment of DsA1 has revealed that DsA1, while generally thought of as hypervariable protein, is in fact highly conserved and most differences are length variability in the PT-repeat region and C-terminal frame shift events (FIG. 12B).

DsA2 sequences have been similarly selected and aligned. DsA1 sequences, specifically when disregarding apparent sequencing/genome assembly artifacts in the public repositories, are also well conserved as in the case of DsA1 (FIG. 12C). The only exception is a certain length variability in the PT-repeat region.

PITP sequences have also been retrieved from the public databases and aligned. Similar to the other two antigens, PITP sequences are also highly conserved among different P. acnes strains (FIG. 12D).

### Generation of Hybrids of DsA1 and DsA2

The 3-domain structure of protein DsA1 (FIG. 7A) was used as a tool to optimize the design of fragments and hybrids to be tested in immunological studies based on the sequence areas that provided some flexibility in terms of start and end of a specific fragment (FIG. 7B). The used ‘swap sites’, i.e. the positions where PROT-22 (DsA1) and PROT-27 (DsA2) sequence regions were switched (as in a protein-cross-over) were chosen within swap regions, areas of least likelihood to interfere with structural elements, specifically alpha helices. The rationale and location of swap regions is also depicted in FIG. 12A.

### Immunological Analysis of Hybrids of DsA1 and DsA2

Several hybrids of DsA1 and DsA2 have been designed to further investigate the most important epitopes of proteins DsA1 and DsA2 and were used as immunogens (as described in Example 1) to induce antibodies capable of equally good specific binding of both DsA1 and DsA2 (as described in Example 1). Immunization with hybrid H4, that includes the functionally most relevant regions confined mostly to the central and N-terminal part of the protein molecule of DsA1 (amino acids 29-145; 278-333) and only the central part of the DsA2 sequence (amino acids 190-321), induced antibodies which had the ability to specifically bind to both full length proteins P022 (DsA1) and P027 (DsA2) in an ELISA assay (as described in Example 1) (FIG. 8). In contrast to antibodies induced by immunization with full length proteins and other fragments and hybrid versions, which specifically bound most highly against themselves but to a much lower extent to the second homologous antigen.

Similarly, in surface binding assay (as described in Example 1) on a large collection of strains covering different phylotypes, antibodies induced by immunization with hybrid H4 had the broadest and most balanced cross-reactivity/cross-type-reactivity (FIG. 9). Antibodies induced by immunization with the other versions of hybrid constructs (e.g. H3 and H5) showed significantly less cross-reactivity (in terms of total MFI levels across the tested phylotypes) compared to H4 (FIG. 9). Additional 20 strains of Type IA1, IC and II were tested in the surface binding assay to evaluate cross-type reactivity and the contribution of H4 to inducing cross-reactive antibodies in comparison to P022 (DsA1) and P027 (Dsa2) (FIG. 16A). On the P. acnes strains that belong to a single MLST phylotype and on which P022 (DsA1) epitopes are less accessible on the cell surface than P027 (DsA2), immunization with H4 led to the increase in the strain-specific as well as total signal compared to P022 (DsA1) (FIG. 16A, 16B, 16E). Likewise, on the strains where P027 (DsA2) epitopes are less accessible on the cell surface compared to P022 (DsA1), immunization with H4 led to the increase in the strain-specific as well as total signal compared to P027 (DsA2) (FIG. 16A, 16C, 16D,16F). FIG. 16A also shows that when a larger number of the strains from MLST phylotype IA1 are tested, these tend to have more DsA1-specific epitopes available to antibody binding, compared to DsA2 and that H4 provides the most balanced cross-type cross-reactivity, that is equally distributed among the strains with the variable expression of one of the DsA homologues.

Biacore analysis, where binding strength of antibodies was evaluated also confirmed that H4 has induced the immune response with the most balanced affinity for both homologues, as evidenced by the equal binding to both P022 (DsA1) and P027 (DsA2); whereas P022 (DsA1) and P027 (DsA2) immunizations led to a disbalanced response with a much more intense and stable binding interaction with the exact corresponding immunogen, compared to the binding to the homologue (FIGS. 19A and 19B).

The ability of different P022 and P027 fragments and hybrids to induce opsonophagocytic killing was evaluated and compared to each other in the OPK assay against Type IA1 strain NCTC737. The bactericidal potency was evidenced by the increased % of reduction in viable cell counts (colony forming units) at different serum dilutions compared to the corresponding dilutions of the control serum, PBS (adjuvant without the protein antigen) (FIG. 18B, FIG. 18C).

### Antigen Stability Test

For the stability assessment the protein samples were submitted to different stress factors like 3 freeze/thaw cycles (FT), storage for 7 days at various temperature conditions: below −20° C. (−20), 2-8° C. (+4), at room-temperature 21-24° C. (RT) and at 37° C. (+37). They were compared to freshly reconstituted protein batches (TO). The proteins were separated by 4-12% SDS-PAGE (Bio-Rad). After separation, a silver staining kit (Pierce) was used to visualize the proteins. Using this kit, proteins are detectable at greater than 0.25 ng per band. Protein sizes were compared to a standard protein gel ladder (Thermo Scientific, 26616) and to a sample that contained bovine serum albumin (200 ng/well loaded). The stained gels were scanned using an office scanner and documented further according to the protocol.

In a stress test over 7 days at elevated temperatures hybrid construct H4 revealed an increased stability when compared to both full length proteins P022 (DsA1) and P027 (DsA2) (FIG. 10A). H4 in contrast to P022 (DsA1) is resistant to proteolytic degradation, and it shows less product related impurities, since it is expressed as a single-band protein in comparison to protein P027 (DsA2) (FIG. 10A).

Recombinant proteins are not only defined by their amino acid sequence but also by the way they are produced. The goal of every production process is to reproducibly reduce the amount of product and process related impurities. It was investigated how different purification strategies and specific sequence alterations (point mutations) would affect the product profiles according to the present invention (FIG. 10B). Different expression products were analyzed by a silver-stained SDS-PAGE under reducing and non-reducing conditions. First recombinant expression products P028V1 and H4-V1 produced by a generic and rudimentary purification strategy were compared to respective products P028-V1′ and H4-V1′ derived from a product specific and optimized process. P028-V1″ additionally was purified under lower temperatures. The main product bands were found in the range of 50 kDa for all P028 variants and 35 kDa for all H4 variants, respectively. This corresponds with the expected masses (respectively 42 and 32 kDa). Taken together, all products derived from the generic process showed a considerable amount of proteins of higher and lower molecular weights. The optimization of the production process helped to considerably reduce the amount of low molecular weight contaminations for P028-V1′ and H4-V1′. Both products nevertheless still showed a considerable amount of high molecular weight impurities, which for H4 disappeared under reducing conditions. While these forms obviously were product related impurities (multimeric forms) for H4, it is not clear why the corresponding “ladder” of higher molecular weight products seen for P028 could not be reduced to the single banded product. Nevertheless, the purity of P028 could be improved by running the identical purification process under reduced temperature, as seen for P028-V1″. However, both proteins appeared under all there conditions as double-banded products.

H4 as well as P028 contain 3 and 2 cysteins, respectively. They can form intra- and intermolecular disulfide bridges. Such covalent bonds affect protein structure and are an important source for protein micro-heterogenity. It was therefore decided to reduce protein complexicity by optimizing respective amino acid sequences. Starting from the N-terminus, the first cysteine in the amino acid sequence in the H4-V2 variant was replaced, as well as all cysteins in the sequences of P028-V7 and H4-V3. While the single mutated form H4-V2 showed the expected decreased amount of intra-molecular disulfide bridges, the amount of inter-molecular bridges had even slightly increased. Importantly, the variants P028-V7 and H4-V3 lacking all cysteins, led to the desired homogenous expression product, consisting mostly of the single-banded product, both under reducing and non-reducing conditions.

### Example 5: Combination of Antigen PITP and Hybrid H4

Animals have been immunized with vaccines containing only antigen P028 (PITP) or only hybrid H4, as well as with vaccines containing the combinations of both P028 (PITP) and hybrid H4, formulated together in a single vaccine according to the method of “Immunization of animals” as described in Example 1. The antibodies raised by this immunization have been tested in a surface binding assay (as described under Example 1).

The studies performed for the present invention in the surface binding assay (as described in Example 1) showed that immunization with a combination of antigen P028 (PITP) and hybrid H4 had induced antibodies with a stronger and broader activity compared to antibodies induced by vaccination with the single antigen or hybrid (FIG. 11A). Antibodies induced by immunization with a combination of antigen P028 (PITP) and hybrid H4 specifically bound to all 6 MLST types of P. acnes (FIG. 11A).

All sera induced after immunization with the single P028 (PITP) antigen or a hybrid H4 and with a combination of antigen P028 (PITP) and hybrid H4 were tested in an OPK assay as described in Example 1. The antibodies induced by immunization with the combination vaccine comprising P028 (PITP) and hybrid H4 were able to induce opsonophagocytic killing of up to 6 MLST types of P. acnes (FIGS. 11B and 11C).

### Example 6: Analysis of Samples from Human Subjects

### Human Clinical Samples

Human serum samples were obtained in the course of Origimm's clinical research studies registered in Austria: study reference number 323423452354 and Ethics Commission approval EK-13-052-0413 (Ethikkommission der Stadt Wien), and in the USA: ClinicalTrials.gov Identifier: NCT04056598. All sera were handled according to internal standard operating procedures, stored in small aliquots at −80C and handled under sterile conditions.

Surface Binding and Opsonophagocytic Killing of Serum Samples from Human Subjects

All human subjects, interdependently from their acne pathology and history of interactions with P. acnes, contain pre-existing antibodies against P. acnes which can be detected in their serum. Serum samples obtained from human subjects were tested in the surface binding assays and OPK assays as described in Example 1. These antibodies target the antigens expressed by live P. acnes and if the epitopes of these antigens are accessible on the P. acnes cell surface, they are able to induce opsonophagocytic killing of the bacterium: in fact, according to the data obtained for the present invention opsonophagocytic killing is the most significant functional activity of the surface-binding antibodies, as evidenced by the significant increase in OPK K50 titer in relation to the increase of the surface binding of the antibodies (FIG. 13A, 13B). The efficiency of the opsonophagocytic killing is influenced not only by the amount of the antibody that binds P. acnes, but also by the type of antigenic epitopes targeted by the antibodies generated in each individual against the surface-accessible and immunogenic epitopes presented by a specific P. acnes strain. These differences can be observed in the comparison of the linearity trends of correlation between the surface binding intensity and OPK K50 titer against the strains NCTC737 (phylotype IA1) (FIG. 13A) and KPA171202 (phylotype IB) (FIG. 13B).

Analysis of Samples Isolated from the Inflamed Acne Lesions (Pustules) from Human Subjects

In the course of a clinical research study (ClinicalTrials.gov Identifier: NCT04056598) material from inflamed lesions (pustules) which were identified on the facial skin of the individual subjects with moderate to severe acne (IGA>2) was isolated. The material was collected with a sterile swab upon sterilizing the skin surface and opening the pustule (acne pimple) with a sterile needle. DNA was extracted directly from the sample, subjected to PCR using SLST-specific primers and the phylotype was determined according to the published method and its associated public database (Scholz et al. 2014).

The data according to the present invention show, that not only IA1, but also other phylotypes are equally able to induce acne, since the direct isolation and analysis of the material from inflamed acne lesions (pustules) has revealed that in many patients it is not IA1, but other phylotypes that are exclusively found in this material (FIG. 14A).

While P. acnes strains isolated from the inflamed lesion of the patient CR086 has been typed according to SLST scheme and found to be F4, which belongs to IA2 phylotype, the P. acnes strains detected in material of the patient CR078 are mainly of the SLST type G1 (phylotype IB) and K1 (phylotype II); therefore this suggest also that mixed infections are possible and not only those induced by a single specific phylope (FIG. 14B). Similar examples of mixed infections can be also seen in other patients, e.g. CR072 (FIG. 14B).

Analysis of Samples Isolated from Skin Pores—Skin Surface.

In the course of a clinical research study (ClinicalTrials.gov Identifier: NCT04056598) also material from the upper part of the skin pores of individual subjects with moderate to severe acne (IGA>2), was isolated. Two different locations were sampled on the face of each individual: forehead and cheek area. To increase DNA yield of skin pore samples, the material was propagated on plates for 4 days at +37° C. in the absence of oxygen, or 3 days in the presence of oxygen at room temperature. The plates were scraped upon incubation. DNA was extracted from the total sample, subjected to PCR using SLST-specific primers and the phylotype was determined according to the published method and its associated public database (Scholz et al. 2014).

The data of the present invention show that not all the strains that colonize the skin pores predominate in the inflamed lesions of the particular individual. For example, the acne lesions of acne patient CR086 were solely colonized by phylotype IA2 (SLST F4), whereas the skin pores from the surrounding facial area of the same patient were colonized by phylotypes IA1, IA2 and IC (SLST A1, F4 and G1) (FIG. 14B). Similar conclusion can be drawn also by comparing the findings among other individuals, e.g. CR078. CR072, CR061 and CR060 (FIG. 14B).

Analysis of Serum Samples from Acne Patient.

In the course of a clinical research study serum from the subjects with moderate to severe acne (IGA>2) was collected and tested for the presence of antibodies against the antigens P028 (PITP), P027 (DsA2), P022 (DsA1) and the hybrid protein H4.

Human sera collected from the individual subjects were tested in an antigen binding ELISA assay as described in Example 1. The antigen binding assay shows a trend that sera of individuals with moderate to severe acne (IGA>2) contain antibodies that more readily bind to the hybrid protein H4 than to the antigens P022 (DsA1), P027 (DsA2) and P028 (PITP) (FIG. 15).

### Example 7: Analysis of Fragments and Derivatives of PITP

### Analysis of Fragments of DsA1 and DsA2

A number of fragments and derivatives of P028 (PITP) have been produced (Table 1A and 1C) and used for animal immunizations (mice) as described in Example 1.

Immunization with fragments which represent primarily or only the ENFD domain (P028F1, P028F16 and P028F18) induced antibodies which had a much better surface binding ability and induced more OPK activity on strains from all 6 MLST types, compared to the fragments that contained the C-terminal half of the protein that included mostly HbD domain (F2, F19 and F20) (FIG. 21A, 21B, 22A-22D). Immunization with P028F7, the fragment which included the ENFD, SR1 and HbD domains (A32-K400) (FIG. 20A), had induced cross-reactive immune response that was slightly lower in comparison to the fragments P028F1, P028F16 and P028F18, but higher compared to the fragments containing mostly HbD domain (F2. F19 and F20) (FIG. 21A, 21B, 22A, 22B); since the presence of additional sequence parts which are less effective inducers of the cross-reactive antibodies had lowered the percentage of the immunorelevant (functional antibodies, according to this invention) in the serum when compared at the same dilution to the serum samples induced by the fragments containing mostly ENFD and N-terminal half of the protein. Although the fragment containing only HbD domain (F2) induced less cross-reactive immune response, the fragment P028F19, which included additional 30 amino acids corresponding to SR2 domain (T232-T430) (FIG. 20A), had induced more cross-reactive immune response compared to F2, suggesting that SR2 can contribute to the immunogenicity of P028F2 (HbD) (FIG. 21A, 21B, 22A-22D). The extension of the fragment P028F2 at the N-terminus, as represented by the fragment P028F20 that includes also the sequence area within the SR1 domain (G172-K400), did not increase P028F2 immunogenicity or cross-reactivity, although the addition of the same sequence region encoding SR1 led to the increased immunogenicity and cross-reactivity of P028F1, evidenced by the increased cross-reactivity of the fragments P028F16 and P028F18 ((FIG. 21A, 21B, 22A-22D).

All fragments and derivatives demonstrated immunogenicity and were capable of inducing antigen-specific immune response in the murine immunization studies according to the present invention, as evidenced by their ability to induce antigen-specific antibodies detectable in ELISA with EC50 titer of at least 10-fold higher compared to the immunization with adjuvant in the absence of the protein antigen (PBS) (FIG. 23A-23C). The serum antibodies induced against PITP fragments and derivatives that included ENFD domain and not HbD domain (P028F1, P028F16, P028F18, P028C12) bound more strongly to P028-V7 (PITP) compared to the fragments that did not include the ENFD domain (P028F2, P028F19, P028F20), as evidenced by the EC50 titer of the pooled sera from five mice immunized with the respective antigens (FIG. 23B), and the overall reactivity in comparison to the binding to the antigen used in immunization (self-antigen) (FIG. 23C).

P028M1 and P028M7, the derivatives of P028 (PITP, Table 1A, FIG. 20C), showed more variable cross-type reactivity as evidenced by the better performance of P028M7 compared to P028M1 against Type II strain HL60PA1 in both surface binding and OPK assays (FIG. 22A, 22B) and slightly more cross-reactivity overall against the strains from additional phylotypes (FIG. 21C, 21D, 22B). Nevertheless, both derivatives P028M1 and P028M2 had overall induced immune responses that were not significantly inferior in comparison to derivative P028-V7 (PITP) (FIG. 21 C, 21D and FIG. 22A). Also, both derivatives P028M1 and P028M2 induced the antibodies that bound the epitopes on the recombinant P028V7 (PITP) in ELISA (FIG. 23B, 23C).

### Example 8: Analysis of Hybrids of PITP and H4

Two hybrids were designed that included H4 and two different fragments of P028 (PITP) (Table 1C, FIG. 20B): P028C12 (H4+P028F1) and P028C13 (H4+P028F2). The P028 fragments F1 or F2 were fused to the C-terminus of H4. Murine serum antibodies raised against the two hybrids were tested in the surface binding assay, OPK and ELISA, to determine their respective cross-reactivity and potency in inducing immunorelevant (functional) antibody response. Much higher level of surface binding to all six MLST phylotypes was achieved with the serum antibodies raised against P028C12, compared to P028C13 (FIG. 21C, 21D). In addition, the cross-reactivity of P028C12 serum antibodies was much better compared to either P028-V7 (PITP) or H4-V3, as evidenced in the increased surface binding as well as opsonophagocytic activity against the six MLST phylotypes (FIG. 21C, 22A, 22B). This increased cross-reactivity and functionality (increased OPK activity) of P028C12 serum antibodies was especially significant in comparison to P028-V7 (PITP) against the strains of MLST types IA1, IA2, IC and II (FIG. 21C, 22A, 22B), whereas on the strains from phylotypes IB and III, P028C12 was comparable to P028-V7 (PITP) (FIG. 21D, 22A) Murine serum antibodies induced by P028C13, however, were inferior in inducingOPK activity compared to H4-V3 fusion partner, which was especially obvious against the strains IA2 and IC (FIG. 22A, 22B), and most of its cross-reactivity detectable in the surface binding was induced by the H4 part of this hybrid (FIG. 21C), or the antibodies induced by additional parts of the hybrid sequence did not lead to significant increase in either surface binding (FIG. 21C, 21D) or OPK activity (FIG. 22B) compared to PITP fusion partner alone; this can be especially observed on the phylotypes IB and III, which do not express DsA1 and DsA2 (FIG. 21D, 22A).

Superiority of P028C12 was confirmed by OPK assay, which induced much better opsonophagocytic killing than P028C13 or P028-V7 (PITP) (FIG. 22B).

Both hybrids were able to induce an antigen-specific immune-response in mice which was shown by ELISA against the antigen itself as well as against P028-V7 (PITP) (FIGS. 23A, 23B and 23C). These data also correspond to the findings that the sera induced by immunization with the fragment P028F1 were also more cross-reactive compared to the sera raised against P028F2, as described in the Example 7. The results additionally demonstrate the importance of selecting not only the right antigens to be combined in the vaccine formulation, but also the importance of selecting the right sequence domains (epitopes) of each antigen in order to design a cross-reactive vaccine with increased effectiveness.

### Example 9: Mode of Action of the Cross-Reactive Vaccine Against P. acnes

The pathogenesis of acne is triggered by the interplay of several factors: genetics and hormonal activity that increases sebum secretion and change the skin environment to become more conductive to P. acnes virulence need to be counterbalanced by the quality of the host immune response. Since the local skin environment, immune system and the colonizing P. acnes strains are unique to each human host, an effective vaccine against P. acnes needs to induce immune responses of wide cross-reactivity to be able to protect the hosts colonized by a variety of different strains and induce a high level of antibodies that are able not only to recognize and bind P. acnes, but to help phagocytes effectively kill and reduce bacterial cell numbers, thereby reducing P. acnes fitness and capacity for damaging host cells, perpetuating inflammation or infection.

In the context of the acne vulgaris indication, where the first signs of infection and inflammation occur inside the skin pores (pilosebaceous hair follicles), it is important to sufficiently increase the serum antibody levels against the vaccine antigens so that they can reach hair follicles in sufficient amount to help regulate P. acnes growth and density inside the hair follicles and support local skin immune defenses during the attempted skin barrier breach. Therefore the mode of action of the P. acnes vaccine, as described in this invention, is to augment pre-existing immunity against P. acnes by inducing and/or increasing the quantity and quality of the opsonizing antibodies against a large number of P. acnes strains within a specific MLST phylotype as well as across various phylotypes, and enhance the efficiency of opsonophagocytic killing by immune cells, leading to more efficient and controlled resolution of the inflammation and infection. The vaccine-induced antibodies can thereby counterbalance the effects of increased sebum secretion that increases P. acnes proliferation and the increased virulence that leads to the skin barrier damage and invasion of the skin tissue surrounding pilosebaceous hair follicles (FIG. 24A).

The additional beneficial effect is expected on the immune cell behaviour in the context of inflammation. An augmented inflammatory response is potentiated by the uncontrolled release of the intracellular contents of the human cells that participate in skin immune defence and which can die by the process of apoptosis or necrosis. In cases where P. acnes is able to resist or escape the phagocytic killing pathway, the inflammation persists not only due to the increased activity of P. acnes, but also due to the additional burden created by the uncontrolled release of the contents from the necrotic phagocytic cells. This continues and perpetuates the stimulation of a local acute inflammatory response, which in more severe cases can lead to a complete loss of hair follicle integrity. On the contrary, when sufficient levels of opsonizing antibodies are present, phagocytes can more efficiently capture and digest bacterium and either leave the site of infection or die by apoptosis, which is a tightly regulated process that occurs under physiological conditions. The anticipated role of antibodies in this process is shown in FIG. 24B.

### Example 10: Epitope Mapping-Methods and Tools; Confirmation of Preferred Epitopes

Different antibodies and controls have been used to further verify and confirm the sequences that contain immunorelevant epitopes which are targeted by the serum antibodies raised against the vaccine antigens, antigen derivatives and fragments. These included polyclonal murine, rabbit and human antibodies or affinity-purified antibody samples. Monoclonal antibodies were developed from the mice immunized against P022-V3, P027-V4 or P028-V1 and used as additional controls.

### Monoclonal Antibodies

For the monoclonal antibody generation BALB/c mice were immunized four-times with P022-V3, P027-V4 or P028-V1. Spleens were aseptically removed, pooled and homogenized. The spleen cells and the SP2/0-Ag14 myeloma cells were fused and antibody secretion of the hybridoma was evaluated in ELISA against the immunization antigen and the host cell proteins. After the second cloning step, stable hybridoma expressing monoclonal antibodies have been selected for further characterization and epitope mapping experiments.

### Linear Epitope Mapping

Origimm prepared the samples and the analysis was performed by the company Pepperprint which uses PEPperCHIP® Peptide Microarrays for the high-resolution epitope mapping of antibodies and sera by translating one or more antigens into overlapping peptides. Incubation of the resulting peptide microarray with an antibody or serum sample as well as suited secondary antibodies gives rise to spot patterns that correlate with the epitope of the given sample. The proteins of interest (P028-V2, P027-V4, P022-V3) are therefore divided into 15 aa-long peptides which overlap by 14 aa with the previous peptide, all of them were spotted on a chip in addition to the positive (HA) and negative (polio) control peptides. Incubation of the peptide microarrays with the antibody and serum samples was performed at different antibody concentrations or at serum dilutions of 1:1000, 1:100 and 1:10 followed by incubation with goat anti-human IgG (H+L) DyLight680, sheep anti-rabbit IgG (H+L) DyLight680 and goat anti-mouse IgG (H+L) DyLight680 for fluorescence detection and control antibody (mouse monoclonal anti-HA (12CA5) DyLight800). The fluorescence signal of the bound antibodies was read-out with a LI-COR Odyssey Imaging System. Quantification of spot intensities and peptide annotations were done with PepSlide® Analyzer. Pre-staining of peptide microarray copies with the various secondary and control antibodies was performed as an additional control to verify that there was no background interaction with the linear peptides that could interfere with the main assays.

### Conformational Epitope Mapping Via Cyclic Constrained Peptides

The PEPperMAP® Conformational Epitope Mappings of murine monoclonal and polyclonal antibodies, human and rabbit serum samples were performed against a P022-V4 fragment (L115-T333), a P027-V4 fragment (V161-E351) or P028-V2 translated into cyclic constrained peptides with peptide lengths of 9 and 13 amino acids and peptide-peptide overlaps of 8 and 12 amino acids. In addition positive (HA) and negative (polio) control peptides were spotted on the chip as well. The conformational peptide microarrays were incubated with the antibody and serum samples at concentrations or dilutions of 1:1000, 1:500, 1:200, 1:100 and 1:50 in the incubation buffer PBS (pH 7.4) with 0.05% Tween 20 and 10% Rockland blocking buffer MB-070) followed by staining with goat anti-human IgG (H+L) DyLight680, sheep anti-rabbit IgG (H+L) DyLight680 and goat anti-mouse IgG (H+L) DyLight680 secondary antibodies for fluorescence detection and control antibody (mouse monoclonal anti-HA (12CA5) DyLight800). The fluorescence signal of the bound antibodies was read-out with a LI-COR Odyssey Imaging System. Quantification of spot intensities and peptide annotation were done with PepSlide® Analyzer. Pre-staining of two conformational peptides of each peptide microarrays with the secondary and control antibodies was performed as an additional control to verify that there was no background interaction with the cyclic constrained peptides that could interfere with the main assays.

### MS Epitope Mapping

For the Cross-linking Mass Spectrometry approach (XL-MS), the mature full length wild-type protein P22-V3, has been chosen for the analysis of the epitopes that we bound by the P022 monoclonal antibodies. The complex of antibody bound to the antigen was stabilized with a mass-labeled chemical crosslinker. Next, the presence of the complex was confirmed using high mass MALDI detection. Since after cross-linking chemistry the Ab/Ag complex is extremely stable, multiple enzymes (five utilized in parallel) and digestion conditions were applied to the complex to provide many different overlapping peptides. Identification of these peptides has been performed using high-resolution Orbitrap™ mass spectrometry and MS/MS techniques. The identification of the crosslinked peptides has been determined using mass tags linked to the crosslinking reagents. After MS/MS fragmentation and data analysis using specific interaction software(s), both epitope and paratope are determined in the same experiment.

### Antigen Fragment Mapping by ELISA

96-well microtiter plates (Nunc-Immuno, Maxisorb) were coated for 18±2 h at 4° C. with 70 μl of recombinant proteins, fragments and hybrids (P022-V3, P027-V4, P022F2-F14, P027-F1 and H2-H5) at concentration between 0.5 μg/ml and 2 μg/ml in PBS, which was optimized for each antigen in preliminary experiments to achieve the best signal to noise ratio. After washing each plate with Wash Buffer (PBS supplemented with 0.1% Tween-20), 100 pI of Blocking Buffer (PBS with 2% BSA and 0.1% Tween-20) were added and incubated for a minimum of 30 min on an orbital shaker to prevent unspecific binding. For the determination of antigen-specific antibody titers. 50 μl of a 5-fold dilution series of heat-inactivated (45 min 56° C.) rabbit or mouse serum was applied to the coated and blocked plate and incubated for 1 hour on an orbital shaker. The primary antibody solution was then removed followed by washing steps and subsequent incubation with 50 μl of the 1/20,000 diluted horse radish peroxidase (HRP)-linked secondary antibody (Peroxidase AffiniPure Goat Anti-Rabbit IgG, Fc fragment specific, Jackson Immuno Research; Peroxidase AffiniPure Goat Anti-Mouse IgG, Fc fragment specific, Jackson Immuno Research). After washing the plates, HRP activity was detected by adding 100 μl of tetramethylbenzidine substrate (TMB, Thermo Fisher) for 10 min incubation, yielding a blue color (Amax=370 nm and 652 nm) that changes to yellow (Amax=450 nm) upon addition of 50 μl of 3N sulphuric acid (Sigma) stop solution. The reaction was quantified using a microtiter plate photometer (Tecan Sunrise). Background values were determined by reading reactions that lacked the primary antibody. The amount of IgG for each test sample was measured in a dilution series in duplicates and the EC50 value was calculated from the sigmoidal curve fit of these measurements by using GraphPad software.

### Dot-Blot Mapping of Overlapping 15-Mer Peptides

Thirteen peptides have been synthesized based on P022F11 sequence as 15-mers with a 5 amino-acid overlap. The overlapping peptides and the full-length P022F11, used as a positive control, have been spotted onto nitrocellulose membrane, blocked by 2% BSA buffer followed by the staining with secondary Peroxidase-conjugated AftiniPure Goat Anti-Mouse IgG (H+L) (Jackson Immuno Research) and detected via ECL substrate in a ChemiDoc Imager.

### Results

These methods and tools described above have led to the confirmation of the most immunorelevant sequence regions and epitopes, which according to the definition in this invention are accessible on the surface of P. acnes and bound by the antibodies that induce opsonophagocytic activity, leading to the reduction of the P. acnes cell numbers by the more effective killing ability of the phagocytic cells, especially those of the granulocytic type (neutrophils) as used on the opsonophagocytic killing assay described in this invention. These methods have also facilitated the identification of the cross-reactive and cross-type reactive epitopes, which according to this invention, are accessible on the surface of at least two or more phylotypes and on at least two or more strains within the same phylotype; therefore, these epitopes are additionally important for the inclusion in a cross-type-reactive P. acnes vaccine.

Accordingly, with these experimental set-up, the following epitopes were identified in the DsA1, DsA2 and PITP proteins, respectively: R32-I41, Q38-K51, R32-K51, T43-K51, Q38-K51, R87-K90+T43-K51, R87-K90+T17-I132 and R87-K90+S234-G250, R87-K90+L246-A260, R87-K90+A256-E270, R87-K90+R266-T277 T117-I132, T117-A127, V128-I132, A144-N157, H146-A160, A156-A170, K166-L180, A176-T190, P186-A198. N181-E191, I216-F224, I216-D225, A226-A24G, S234-G250, I251-I263, I251-I267, I264-P271, P236-G250, L246-A260, A256-E27G, S234-G250, I251-L267, A268-L280, R266-T277, T285-R286+I216-F224, T285-R286+I264-P271, T285-R286+V289-K296, T285-R286+V289-K296, T285-R286+A144-N157, A310-D313+T285-R286, T285-D290, T285-D290+V291-T300, T285-D290+A301-E307. V291-T300, A301-E307, T285-T300, A301-E307, R286-D290+V291-T300, R286-D290+A301-E307, R286-T300, V289-K296, A310-D313+I216-F224, A310-D313+I264-P271, A310-D313+T285-R286, A310-D313+R286-D290, A310-D313+V289-K296, A310-D313+V289-K296, A310-D313+T285-T300, A310-D313+A144-N157, A310-D313+T285-R286, A310-D313+T285-D290, A310-D313+T293-E307, T285-D290, V291-T300, T293-E307, A301-E307 of DsA1; L152-Q166, G190-P230, I199-D208, A218-I237, P230-Q244, I231-A270, H254-A270, A271-S279, A271-R310, L311-T321, L311-K323, V333-Q347, A218-P230, I231-I237, H254-H262, Q256-H262, E261-D269, D269-S279, K313-K323, of DsA2; D79-T90, E73-D85, R43-150, P68-Y75, P86-E92, 139-G45, Y84-D89, F81-D89, D79-D89, T37-E44, E73-W98, E73-F81, D79-T90, P72-F81, A129-F138, D120-Q134, F111-D120. F132-G147, D152-E165, R115-F123, D120-K128, P131-F138, N181-E191, T143-T159, P116-T124, P131-D137, P131-D137, T175-C231, Q198-K203, P179-K185, G200-Q210, K174-A188, K174-K185, P201-Q209, P183-P201, P183-K191, K185-P195, R164-S180, E165-S180, K185-S190, V193-N202, V193-G200, K203-P208, R216-T225, R216-R224, P173-K191, K197-K203, P168-T175, K185-K203, R164-K174, T175-V193, S250-N261, D287-S300. K340-V347, D338-F352, D338-D348, S285-P288+G305-L314, S285-P288+H306-L314, S285-P288+T342-T351, S285-P288+D338-D348, D287-S300, T342-T351, D338-D348, H306-L314, G305-L314, G364-K375, R382-E399, V367-G373, A383-L390, T342-T351, M387-T395, E385-T392, V401-V410, N404-A409. G416-L427, L396-V410, T406-1415, D417-G424, V407-D418, V407-V414, K42I-V429, S419-T430, D408-1415, T406-V414, of PITP.

For example, the following specifically preferred epitopes were identified in DsA1 (as suitable minimum sequence (e.g. to be provided in a peptide vaccine) and as preferable length to be included in a peptide-based vaccine; as linear epitopes, as conformational epitopes (“c”) and/or as linerar/conformational epitope (“1/c”)):
R32-I41 (minimum), M28-D44 (preferred);
T43-K51 (minimum), M1[del2-27]S28-K51 (preferred), c;
R87-K90 (minimum), P86-A93 (preferred), P86-G96 (preferred), c;
T117-A125 (minimum), T117-I132 (preferred), c;
A144-N157 (minimum), H146-A160 (minimum), l/c;
A156-A170 (minimum);
K166-L180 (minimum);
A176-T190 (minimum);
N181-E191 (minimum), c;
P186-A198 (minimum);
I216-F224 (minimum), I216-D225 (preferred), c;
A226-A240 (minimum);
P236-G250 (minimum), S234-G250 (preferred) l/c;
L246-A260 (preferred);[
I251-I263 (minimum), I251-L267 (preferred), l/c;
I264-P271 (minimum), c;
R266-T277 (minimum);
A268-L280 (minimum), l/c;
T285-D290 (minimum), c;
R286-D290 (minimum), c;
V289-K296 (minimum), R286-K296 (preferred), c;
V291-T300 (minimum), c;
A301-E307 (minimum), c;
A310-D313 (minimum); A291-D313 (preferred), c.

### Example 11: Differential Scanning Fluorimetry to Determine pH Stability of Polypeptides

pH stability may be determined e.g. by the Prometheus technique (Chattopadhyay et al Prot. Sci. 28 (2019), I127-1134; Martin et al., 2014-NanoTemper Technologies GmbH-Application_Note_NT-PR-001_-_Thermal_Unfolding; Krakowiak et al., J. Biol. Phys. 45 (2019), 161-172) using differential scanning fluorimetry (see example section). Differential scanning fluorimetry allows the measurement of thermal unfolding or chemical denaturation under native and label-free conditions by detecting changes in protein intrinsic fluorescence during a thermal ramp or in the presence of a chemical denaturant. For thermal unfolding, the temperatures of the transitions from the folded to the unfolded state are determined. Higher transition temperatures correspond to higher thermal stability.

Polypeptide samples were diluted 1:10 from a 79 μM protein stock (1×PBS pH 7.6) in one of the following buffers: 50 mM citric acid/phosphate (Acros, 110450010; Sigma, S3264) 125 mM NaCl (Sigma, S7653). 50 mM phosphate (Sigma, S3264; Sigma. S5011) 125 mM NaCl. or 50 mM Tris (Sigma, T6066) 125 mM NaCl. The buffers were used at various pH, which was set at room temperature. The samples were transferred to glass capillaries (NanoTemper Technologies GmbH, #PR-C006) and inserted into a Prometheus NT.48 (NanoTemper Technologies GmbH). Melting curves were determined by the measurement of protein intrinsic fluorescence at 330 nm and 350 nm after excitation at 280 nm. The starting temperature of 20° C., the end temperature of 95° C., the heating rate of 1° C. per minute, and the excitation power of 100% were set in the ThermControl software (NanoTemper Technologies GmbH). The data was analyzed with PR.Stability Analysis software (NanoTemper Technologies GmbH).

TABLE 1A
Antigens used in this study, including annotations and sequence-relevant information.

| | | | | Suggested most | | | | | |
|---|---|---|---|---|---|---|---|---|---|
| | | | Annotations commonly | correct annotation | | | | | |
| | | | found assigned to sequence | (used according | | N-terminal | | | Uniprot # or |
| | Generic name | Generic name | variants (ordered by | to the present | His-tag | Methionine | Protein sequence | Rec. protein | Sequence |
| ORIGIMM ID | (Public database) | (Literature) | prominence) | invention) | (yes/no) | added | aa (start-end) | sequence origin | source |
| antigen | single proteins | | | | | | polypeptide | P. acnes | |
| (represented by | | | | | | | | strain | |
| a specific | | | | | | | | | |
| sequence) | | | | | | | | | |
| P002 | cAMP factor | CAMP factor 2 | CAMP factor pore-forming | CAMP factor 2 | yes | yes | 27-267 | KPA171202 | Q6A9X2 |
| | | | toxin 2; Tat pathway signal | | | | | | |
| | | | sequence domain protein; | | | | | | |
| | | | fimbrial protein | | | | | | |
| P005 | Glyceraldehyde-3- | Glyceraldehyde-3- | type I glyceraldehyde-3- | Type I glyceraldehyde- | yes | no | 1-335 | SK137 | D4HCY6 |
| | phosphate dehydrogenase | phosphate dehydrogenase | phosphate dehydrogenase | 3-phosphate | | | | | |
| | | | | dehydrogenase | | | | | |
| P018 | Repressor | Putative DtxR-like | metal-dependent transcriptional | Putative DtxR family | yes | no | 1-232 | KPA171202 | Q6AAH9 |
| | | protein | regulator; iron dependent | iron dependent | | | | | |
| | | | repressor, N-terminal DNA | repressor | | | | | |
| | | | binding domain protein; | | | | | | |
| | | | repressor | | | | | | |
| P022 | Uncharacterized protein | DsA1 | hypothetical protein; adhesin; | Dermatan sulfate | yes | yes | 2-405 | KPA171202 | Q6A5X9 |
| P022-V1 | | | adhesion or S-layer protein | binding adhesin DsA1 | yes | no | 1-418 | HL096PA1 | CP003293.1 |
| | | | | | | | | | (nucleotides |
| | | | | | | | | | 2236421 to |
| | | | | | | | | | 2237674) |
| P022-V2 | | | | | yes | yes | 29-405 | KPA171202 | Q6A5X9 |
| P022-V3 | | | | | no | yes | 29-405 | KPA171202 | Q6A5X9 |
| P022-V4 | | | | | yes | yes | 29-405 (C53S) | KPA171202 | Q6A5X9 |
| P027 | Uncharacterized protein | DsA2 | hypothetical protein; adhesin; | Dermatan sulfate | yes | yes | 32-463 | KPA171202 | Q6A5P9 |
| P027-V1 | | | adhesion or S-layer protein | binding adhesin DsA2 | yes | no | 1-463 | KPA171202 | Q6A5P9 |
| P027-V2 | | | | | yes | no | 1-455 | ATCC 6919 | CP044255.1 |
| | | | | | | | | | (nucleotides |
| | | | | | | | | | 2326799 to |
| | | | | | | | | | 2328163) |
| P027-V3 | | | | | yes | ves | 72-463 | KPA171202 | Q6A5P9 |
| P027-V4 | | | | | no | yes | 72-463 | KPA171202 | Q6A5P9 |
| P027-V5 | | | | | yes | yes | 72-463 (C97S) | KPA171202 | Q6A5P9 |
| P028 | Conserved protein, | HtaA-like | htaa family protein; htaa; HtaA | Putative iron transport | yes | yes | 32-467 | KPA171202 | Q6A9N1 |
| P028-V1 | putative for Fe-transport | | domain protein; htaa protein; | protein (PITP) | no | yes | 32-430 | | |
| P028-V2 | | | HtaA | | no | yes | 32-467 | | |
| P028-V3 | | | | | no | yes | 32-391 (w/o 174-239) | | |
| P028-V4 | | | | | no | yes | 32-391 | | |
| P028-V5 | | | | | no | yes | 32-430 (C231S) | | |
| P028-V6 | | | | | no | yes | 32-430 (C402S) | | |
| P028-V7 | | | | | no | yes | 32-430 (C231S, | | |
| | | | | | | | C402S) | | |
| P028-V8 | | | | | yes | yes | 32-430 (C231S, | | |
| | | | | | | | C402S) | | |
| P028M1 | | | | | yes | yes | 32-430 (H306A) | | |
| P028M7 | | | | | yes | yes | 32-430 (Y254A, | | |
| | | | | | | | H306A) | | |
| P032 | Translation initiation factor | Translation initiation | Translation initiation factor IF-2 | Translation initiation | yes | no | 1-964 | SK137 | D4HF37 |
| | IF-2 | factor IF-2 | | factor IF-2 | | | | | |
| P035 | Malate dehydrogenase | Malate dehydrogenase | Malate dehydrogenase | Malate dehydrogenase | yes | no | 1-327 | SK137 | D4H9E8 |
| P042 | TED domain-containing | ? | TQXA domain-containing | TED domain containing | yes | yes | 33-466 | KPA171202 | Q6A720 |
| | protein | | protein; hypothetical protein | putative gram-positive | | | | | |
| | | | | cell-surface protein | | | | | |
| P046 | Probable endolytic | ? | septal ring lytic | Probable endolytic | yes | yes | 2-343 | KPA171202 | Q6A5T2 |
| | peptidoglycan | | transglycosylase RlpA family | peptidoglycan | | | | | |
| | transglycosylase RlpA | | protein | transglycosylase RlpA | | | | | |
| P068 | Uncharacterized protein | RoxP | hypothetical protein; RoxP | Heme dependent | yes | yes | 23-161 | KPA171202 | Q6A6F6 |
| | | | | antioxidant protein | | | | | |
| | | | | RoxP | | | | | |
| P069 | Outer membrane | ? | methionine ABC transporter | NLPA family | yes | yes | 46-320 | KPA171202 | Q6A6X7 |
| | lipoprotein | | substrate-binding protein; | lipoprotein, putative | | | | | |
| | | | NLPA lipoprotein; hypothetical | methionine ABC | | | | | |
| | | | protein | transporter substrate- | | | | | |
| | | | | binding protein | | | | | |
| P070 | ABC transporter, ATP- | ? | ABC transporter, ATP-binding | ABC transporter, ATP- | yes | yes | 2-244 | SK137 | D4HAH2 |
| | binding protein | | protein | binding protein | | | | | |
| P071 | HtaA-like surface protein | HtaA | HtaA domain protein; htaa; | Putative iron | yes | yes | 34-523 | ATCC 6919 | Q06WK8 |
| | | | HtaA-like surface protein | transporter HtaB-like | | | | | |

TABLE 1B
Fragments and hybrid constructs used in the present invention and the associated sequence-relevant information.

| | | | | Suggested | | | | |
|---|---|---|---|---|---|---|---|---|
| | | | Annotations | most | | | | |
| | | | commonly | correct | | | | |
| | | | found | annotation | | | | |
| | | | assigned to | (used | | | | |
| | | | sequence | according | | | Rec. | Uniprot # |
| | | Generic | variants | to the | | | protein | or |
| Origimm | Fragment | name | (ordered by | present | His-tag | aa | sequence | Sequence |
| ID | Origin | (Literature) | prominence) | invention) | (yes/no) | (start-end) | origin | source |
| P022F1 | P022 | DsA1 | Hypothetical | Dermatan | Yes | 2-142 | KPA171202 | Q6A5X9 |
| P022F2 | | | protein; | sulfate | Yes | 96-236 | | |
| P022F3 | | | adhesin or | binding | Yes | 208-363 | | |
| P022F4 | | | adhesion or | adhesin | Yes | 271-405 | | |
| P022F5 | | | S-layer | DsA1 | Yes | 2-236 | | |
| P022F6 | | | protein | | Yes | 2-363 | | |
| P022F7 | | | | | Yes | 96-405 | | |
| P022F8 | | | | | Yes | 23-320 | | |
| P022F9 | | | | | Yes | 29-333 | | |
| P022F10 | | | | | Yes | 29-145 | | |
| P022F11 | | | | | Yes | 146-277 | | |
| P022F12 | | | | | Yes | 194-363 | | |
| P022F13 | | | | | Yes | 194-405 | | |
| P022F14 | | | | | Yes | 29-320 | | |
| P027F1 | P027 | DsA2 | Hypothetical | Dermatan | Yes | 190-321 | KPA171202 | Q6A5P9 |
| | | | protein; | sulfate | | | | |
| | | | adhesin or | binding | | | | |
| | | | adhesion or | adhesin | | | | |
| | | | S-layer | DsA2 | | | | |
| | | | protein | | | | | |
| H2 | P022/P027 | DsA1/ | | | Yes | 146-277/ | KPA171202 | Q6A5X9/ |
| | | DsA2 | | | | 187-321 | | Q6A5P9 |
| H3 | P022/P027 | | | | Yes | 29-145/ | KPA171202 | Q6A5X9/ |
| | | | | | | 72-189 | | Q6A5P9 |
| H4 | P022/P027/ | DsA1/ | | | Yes | 29-145/ | KPA171202 | Q6A5X9/ |
| | P022 | DsA2/ | | | | 190-321/ | | Q6A5P9/ |
| | | DsA1 | | | | 278-333 | | Q6A5X9 |
| H4-V1 | P022/P027/ | | | | No | 29-145/ | KPA171202 | Q6A5X9/ |
| | P022 | | | | | 190-321/ | | Q6A5P9/ |
| | | | | | | 278-333 | | Q6A5X9 |
| H4-V2 | P022/P027/ | | | | No | 29-145 | KPA171202 | Q6A5X9/ |
| | P022 | | | | | (C53S)/ | | Q6A5P9/ |
| | | | | | | 190-321/ | | Q6A5X9 |
| | | | | | | 278-333 | | |
| | | | | | | (C319S, | | |
| | | | | | | C321P) | | |
| H4-V3 | P022/P027/ | | | | No | 29-145 | KPA171202 | Q6A5X9/ |
| | P022 | | | | | (C53S)/ | | Q6A5P9/ |
| | | | | | | 190-321/ | | Q6A5X9 |
| | | | | | | 278-333 | | |
| | | | | | | (C319S, | | |
| | | | | | | C321P) | | |
| H4-V4 | P022/P027/ | DsA1/ | | Yes | | 29-145 | KPA171202 | Q6A5X9/ |
| | P022 | DsA2/ | | | | (C53S)/ | | Q6A5P9/ |
| | | DsA1 | | | | 190-321/ | | Q6A5X9 |
| | | | | | | 278-333 | | |
| | | | | | | (C319S, | | |
| | | | | | | C321P) | | |
| H4-V5 | P027/P022 | DsA2/ | | Yes | | 190-321/ | KPA171202 | Q6A5P9/ |
| | | DsA1 | | | | 278-333 | | Q6A5X9 |
| P022F1 | P022 | DsA1 | hypothetical | Dermatan | yes | 2-142 | KPA171202 | Q6A5X9 |
| P022F2 | | | protein; | sulfate | yes | 96-236 | | |
| P022F3 | | | adhesin; | binding | yes | 208-363 | | |
| P022F4 | | | adhesion or | adhesin | yes | 271-405 | | |
| P022F5 | | | S-layer | DsA1 | yes | 2-236 | | |
| P022F6 | | | protein | | yes | 2-363 | | |
| P022F7 | | | | | yes | 96-405 | | |
| P022F8 | | | | | yes | 23-320 | | |
| P022F9 | | | | | yes | 29-333 | | |
| P022F10 | | | | | yes | 29-145 | | |
| P022F11 | | | | | yes | 146-277 | | |
| P022F12 | | | | | yes | 194-363 | | |
| P022F13 | | | | | yes | 194-405 | | |
| P022F14 | | | | | yes | 29-320 | | |
| P027F1 | P027 | DsA2 | hypothetical | Dermatan | yes | 190-321 | KPA171202 | Q6A5P9 |
| | | | protein; | sulfate | | | | |
| | | | adhesin; | binding | | | | |
| | | | adhesion or | adhesin | | | | |
| | | | S-layer | DsA2 | | | | |
| | | | protein | | | | | |
| H2 | P022/P027 | DsA1/ | | | yes | 146-277/ | | Q6A5X9/ |
| | | DsA2 | | | | 187-321 | | Q6A5P9 |
| H3 | P022/P027 | | | | yes | 29-145/ | | Q6A5X9/ |
| | | | | | | 72-189 | | Q6A5P9 |
| H4 | P022/P027/ | DsA1/ | | | yes | 29-145/ | KPA171202 | Q6A5X9/ |
| | P022 | DsA2/ | | | | 190-321/ | | Q6A5P9/ |
| | | DsA1 | | | | 278-333 | | Q6A5X9 |
| H4-V1 | P022/P027/ | | | | no | 29-145/ | | Q6A5X9/ |
| | P022 | | | | | 190-321/ | | Q6A5P9/ |
| | | | | | | 278-333 | | Q6A5X9 |
| H4-V2 | P022/P027/ | | | | no | 29-145 | | Q6A5X9/ |
| | P022 | | | | | (C53S)/ | | Q6A5P9/ |
| | | | | | | 190-321/ | | Q6A5X9 |
| | | | | | | 278-333 | | |
| H4-V3 | P022/P027/ | | | | no | 29-145 | | Q6A5X9/ |
| | P022 | | | | | (C53S)/ | | Q6A5P9/ |
| | | | | | | 190-321/ | | Q6A5X9 |
| | | | | | | 278-333 | | |
| | | | | | | (C319S, | | |
| | | | | | | C321P) | | |
| H4-V4 | P022/P027/ | DsA1/ | | | yes | 29-145 | | Q6A5X9/ |
| | P022 | DsA2/ | | | | (C53S)/ | | Q6A5P9/ |
| | | DsA1 | | | | 190-321/ | | Q6A5X9 |
| | | | | | | 278-333 | | |
| | | | | | | (C319S, | | |
| | | | | | | C321P) | | |
| H5 | P027/P022 | DsA2/ | | | yes | 190-321/ | | Q6A5P9 |
| | | DsA1 | | | | 278-333 | | Q6A5X9 |

TABLE 1C
Fragments and hybrid constructs in connection with PITP used in the present invention and the
associated sequence-relevant information.

| | | | | Suggested | | | | |
|---|---|---|---|---|---|---|---|---|
| | | | Annotations | most | | | | |
| | | | commonly | correct | | | | |
| | | | found | annotation | | | | |
| | | | assigned to | (used | | | | |
| | | | sequence | according | | | Rec. | Uniprot |
| | Frag- | Generic | variants | to the | | Aa | protein | # or |
| Origimm | ment | name | (ordered by | present | His-tag | (start- | sequence | Sequence |
| ID PCT | origin | (Literature) | prominence) | invention) | (yes/no) | end) | origin | source |
| P028F1 | P028- | PITP | Putative | Putative | Yes | 32-164 | KPA171202 | Q6A9NI |
| P028F2 | V7 | | iron | iron | Yes | 238-400 | | |
| P028F7 | | | transport | transport | Yes | 32-400 | | |
| P028F16 | | | protein | protein | Yes | 32-177 | | |
| P028F18 | | | (PITP) | (PITP) | Yes | 32-234 | | |
| P028F18 | | | | | yes | 172-400 | | |
| P028C12 | H4- | DsA1/ | | | Yes | 29-145 | KPA171202 | Q6A5X9/ |
| | P028 | DsA2/DsA1/ | | | | (C53S)/ | | Q6A5P9/ |
| | F1 | PITP | | | | 190-321/ | | Q6A5X9/ |
| | | | | | | 278-333 | | Q6A9N1 |
| | | | | | | (C319S, | | |
| | | | | | | C321P)/ | | |
| | | | | | | 32-164 | | |
| P028C13 | H4- | DsA1/ | | | Yes | 29-145 | KPA171202 | Q6A5X9/ |
| | P028 | DsA2/DsA1/ | | | | (C53S)/ | | Q6A5P9/ |
| | F2 | PITP | | | | 190-321/ | | Q6A5X9/ |
| | | | | | | 278-333 | | Q6A9N1 |
| | | | | | | (C319S, | | |
| | | | | | | C321P)/ | | |
| | | | | | | 238-400 | | |

TABLE 2
Strain collection used in the present invention.

| | | | | | Isolation | Source | Phylogenetic | Alternative |
|---|---|---|---|---|---|---|---|---|
| No. | Strain | Phylotype | Ribotype | Source | site | reference | Analysis | name* |
| 1 | NCTC737 | IA1 | 1 | NCTC | acne skin | Douglas et | McDowell et | |
| | | | | | | al. and | al. 2012 | |
| | | | | | | Gunter | | |
| | | | | | | 1946 | | |
| 2 | PV10 | IA1 | 1 | Queen's | acne skin | McDowell | McDowell et | |
| | | | | University | | et al. 2012 | al. 2012 | |
| | | | | Belfast | | | | |
| 3 | P.acn25 | IA1 | 1 | Queen's | corneal | McDowell | McDowell et | |
| | | | | University | scrape | et al. 2012 | al. 2012 | |
| | | | | Belfast | | | | |
| 4 | A002 | IA1 | | Origimm | acne skin | | Kuehnast et | IA1-s14 |
| | | | | study | | | al. 2018 | |
| 5 | A003 | IA1 | | Origimm | acne skin | | Kuehnast et | IA1-s15 |
| | | | | study | | | al. 2018 | |
| 6 | A011 | IA1 | | Origimm | acne skin | | Kuehnast et | IA1-s16 |
| | | | | study | | | al. 2018 | |
| 7 | Hung.#4 | IA1 | | University | acne skin | | | |
| | | | | of Szeged | | | | |
| 8 | IAI 006 | IA1 | | Pro-Implant | plates & | | Kuehnast et | IA1-i1 |
| | | | | Foundation | screws | | al. 2018 | |
| 9 | IAI 011 | IA1 | | Pro-Implant | Osteo- | | Kuehnast et | IA1-i4 |
| | | | | Foundation | synthesis | | al. 2018 | |
| 10 | IAI 013 | IA1 | | Pro-Implant | clavicula | | Kuehnast et | IA1-i5 |
| | | | | Foundation | | | al. 2018 | |
| 11 | IAI 016 | IA1 | | Pro-Implant | knee endo- | | Kuehnast et | IA1-i7 |
| | | | | Foundation | prosthesis | | al. 2018 | |
| 12 | IAI 020 | IA1 | | Pro-Implant | implant | | Kuehnast et | IA1-i8 |
| | | | | Foundation | screws | | al. 2018 | |
| 13 | A010 SK1 | IA1 | | Origimm | acne skin | | Kuehnast et | IA1-s5 |
| | | | | study | | | al. 2018 | |
| 14 | A015 SK1 | IA1 | | Origimm | acne skin | | Kuehnast et | IA1-s7 |
| | | | | study | | | al. 2018 | |
| 15 | A016P SK1 | IA1 | | Origimm | acne skin | | Kuehnast et | IA1-s9 |
| | | | | study | | | al. 2018 | |
| 16 | A016H SK2 | IA1 | | Origimm | acne skin | | Kuehnast et | IA1-s10 |
| | | | | study | | | al. 2018 | |
| 17 | TW SK3 | IA1 | | Origimm | Skin | | Kuehnast et | IA1-s13 |
| | | | | study | | | al. 2018 | |
| 18 | IAI029 | IA1 | | Pro-Implant | Ventriculo- | | Origimm | |
| | | | | Foundation | peritoneal | | study | |
| | | | | | shunt | | | |
| 19 | SK 137 | IA1 | 1 | BEI | Skin | NIH as | McDowell et | |
| | | | | Ressources | | part of | al. 2012 | |
| | | | | | | HMP | | |
| 20 | SK 182 | IA1 | 1 | BEI | Skin | NIH as | McDowell et | |
| | | | | Ressources | | part of | al. 2012 | |
| | | | | | | HMP | | |
| 21 | HL005PA1 | IA1 | 4 | BEI | Skin | NIH as | Tomida et al. | |
| | | | | Ressources | | part of | 2013; | |
| | | | | | | HMP | McDowell et | |
| | | | | | | | al. 2012 | |
| 22 | HL005PA2 | IA1 | 1 | BEI | Skin | NIH as | Tomida et al. | |
| | | | | Ressources | | part of | 2013; | |
| | | | | | | HMP | McDowell et | |
| | | | | | | | al. 2012 | |
| 23 | HL025PA1 | IA1 | 1 | BEI | Skin | NIH as | Tomida et al. | |
| | | | | Ressources | | part of | 2013; | |
| | | | | | | HMP | McDowell et | |
| | | | | | | | al. 2012 | |
| 24 | HL036PA1 | IA1 | 532 | BEI | Skin | NIH as | Tomida et al. | |
| | | | | Ressources | | part of | 2013; | |
| | | | | | | HMP | McDowell et | |
| | | | | | | | al. 2012 | |
| 25 | HL043PA1 | IA1 | 5 | BEI | acne skin | NIH as | Tomida et al. | |
| | | | | Ressources | | part of | 2013; | |
| | | | | | | HMP | McDowell et | |
| | | | | | | | al. 2012 | |
| 26 | HL043PA2 | IA1 | 5 | BEI | acne skin | NIH as | Tomida et al. | |
| | | | | Ressources | | part of | 2013; | |
| | | | | | | HMP | McDowell et | |
| | | | | | | | al. 2012 | |
| 27 | HL045PA1 | IA1 | 4 | BEI | acne skin | NIH as | Tomida et al. | |
| | | | | Ressources | | part of | 2013; | |
| | | | | | | HMP | McDowell et | |
| | | | | | | | al. 2012 | |
| 28 | HL053PA1 | IA1 | 4 | BEI | acne skin | NIH as | Tomida et al. | |
| | | | | Ressources | | part of | 2013; | |
| | | | | | | HMP | McDowell et | |
| | | | | | | | al. 2012 | |
| 29 | HL072PA1 | IA1 | 5 | BEI | acne skin | NIH as | Tomida et al. | |
| | | | | Ressources | | part of | 2013; | |
| | | | | | | HMP | McDowell et | |
| | | | | | | | al. 2012 | |
| 30 | HL002PA3 | IA1 | 1 | BEI | acne skin | NIH as | Tomida et al. | |
| | | | | Ressources | | part of | 2013; | |
| | | | | | | HMP | McDowell et | |
| | | | | | | | al. 2012 | |
| 31 | HL005PA3 | IA1 | 1 | BEI | Skin | NIH as | Tomida et al. | |
| | | | | Ressources | | part of | 2013; | |
| | | | | | | HMP | McDowell et | |
| | | | | | | | al. 2012 | |
| 32 | HL007PA1 | IA1 | 4 | BEI | Skin | NIH as | Tomida et al. | |
| | | | | Ressources | | part of | 2013; | |
| | | | | | | HMP | McDowell et | |
| | | | | | | | al. 2012 | |
| 33 | HL013PA2 | IA1 | 1 | BEI | acne skin | NIH as | Tomida et al. | |
| | | | | Ressources | | part of | 2013; | |
| | | | | | | HMP | McDowell et | |
| | | | | | | | al. 2012 | |
| 34 | HL020PA1 | IA1 | 1 | BEI | acne skin | NIH as | Tomida et al. | |
| | | | | Ressources | | part of | 2013; | |
| | | | | | | HMP | McDowell et | |
| | | | | | | | al. 2012 | |
| 35 | HL036PA3 | IA1 | 1 | BEI | Skin | NIH as | Tomida et al. | |
| | | | | Ressources | | part of | 2013; | |
| | | | | | | HMP | McDowell et | |
| | | | | | | | al. 2012 | |
| 36 | HL038PA1 | IA1 | 4 | BEI | acne skin | NIH as | Tomida et al. | |
| | | | | Ressources | | part of | 2013; | |
| | | | | | | HMP | McDowell et | |
| | | | | | | | al. 2012 | |
| 37 | HL046PA2 | IA1 | 1 | BEI | acne skin | NIH as | Tomida et al. | |
| | | | | Ressources | | part of | 2013; | |
| | | | | | | HMP | McDowell et | |
| | | | | | | | al. 2012 | |
| 38 | HL056PA1 | IA1 | 4 | BEI | Skin | NIH as | Tomida et al. | |
| | | | | Ressources | | part of | 2013; | |
| | | | | | | HMP | McDowell et | |
| | | | | | | | al. 2012 | |
| 39 | HL078PA1 | IA1 | 1 | BEI | Skin | NIH as | Tomida et al. | |
| | | | | Ressources | | part of | 2013; | |
| | | | | | | HMP | McDowell et | |
| | | | | | | | al. 2012 | |
| 40 | HL002PA2 | IA1 | 1 | BEI | acne skin | NIH as | Tomida et al. | |
| | | | | Ressources | | part of | 2013; | |
| | | | | | | HMP | McDowell et | |
| | | | | | | | al. 2012 | |
| 41 | HL036PA2 | IA1 | 532 | BEI | Skin | NIH as | Tomida et al. | |
| | | | | Ressources | | part of | 2013; | |
| | | | | | | HMP | McDowell et | |
| | | | | | | | al. 2012 | |
| 42 | HL027PA2 | IA1 | 1 | BEI | Skin | NIH as | Tomida et al. | |
| | | | | Ressources | | part of | 2013; | |
| | | | | | | HMP | McDowell et | |
| | | | | | | | al. 2012 | |
| 43 | HL086PA1 | IA1 | 8 | BEI | Skin | NIH as | Tomida et al. | |
| | | | | Ressources | | part of | 2013; | |
| | | | | | | HMP | McDowell et | |
| | | | | | | | al. 2012 | |
| 44 | HL053PA2 | IA-1 | 8 | BEI | acne skin | NIH as | Tomida et al. | |
| | | | | Ressources | | part of | 2013; | |
| | | | | | | HMP | McDowell et | |
| | | | | | | | al. 2012 | |
| 45 | HL063PA1 | IA-1 | 1 | BEI | Skin | NIH as | Tomida et al. | |
| | | | | Ressources | | part of | 2013; | |
| | | | | | | HMP | McDowell et | |
| | | | | | | | al. 2012 | |
| 46 | HL110PA1 | IA-1 | 8 | BEI | acne skin | NIH as | Tomida et al. | |
| | | | | Ressources | | part of | 2013; | |
| | | | | | | HMP | McDowell et | |
| | | | | | | | al. 2012 | |
| 47 | IAI027 | IA1 | | Pro-Implant | vertebral | | Origimm | |
| | | | | Foundation | body | | study | |
| | | | | | implant | | | |
| 48 | IAI030 | IA1 | | Pro-Implant | Clavicula | | Origimm | |
| | | | | Foundation | | | study | |
| 49 | IAI032 | IA1 | | Pro-Implant | ventriculo | | Origimm | |
| | | | | Foundation | peritoneal | | study | |
| | | | | | shunt | | | |
| 50 | IAI036 | IA1 | | Pro-Implant | pacemaker | | Origimm | |
| | | | | Foundation | | | study | |
| 51 | IAI037 | IA1 | | Pro-Implant | K-TEP | | Origimm | |
| | | | | Foundation | | | study | |
| 52 | IAI039 | IA1 | | Pro-Implant | implant | | Origimm | |
| | | | | Foundation | screws | | study | |
| 53 | IAI043 | IA1 | | Pro-Implant | shunt | | Origimm | |
| | | | | Foundation | | | study | |
| 54 | IAI044 | IA1 | | Pro-Implant | knee | | Origimm | |
| | | | | Foundation | endo- | | study | |
| | | | | | prosthesis | | | |
| 55 | IAI046 | IA1 | | Pro-Implant | knee | | Origimm | |
| | | | | Foundation | endo- | | study | |
| | | | | | prosthesis | | | |
| 56 | A017-#4 | IA1 | | Origimm | acne skin | | Origimm | |
| | | | | study | | | study | |
| 57 | A019-#2 | IA1 | | Origimm | acne skin | | Origimm | |
| | | | | study | | | study | |
| 58 | MBESK1 | IA1 | | Origimm | skin | | Kuehnast et | IA1-s19 |
| | | | | study | | | al. 2018 | |
| 59 | H005 SK2 | IA1 | 1 | Origimm | skin | | Kuehnast et | IA1-s2 |
| | | | | study | | | al. 2018 | |
| 60 | APSK 1 | IA1 | | Origimm | skin | | Kuehnast et | IA1-s18 |
| | | | | study | | | al. 2018 | |
| 61 | A017-#2 | IA1 | | Origimm | acne skin | | Origimm | |
| | | | | study | | | study | |
| 62 | P.acn31 | IA2 | 3 | Queen's | aqueous | McDowell | Vorös et al. | |
| | | | | University | humour | et al. 2011 | 2012; | |
| | | | | Belfast | | | Barnard et al. | |
| | | | | | | | 2015 | |
| 63 | IAI 018 | IA2 | | Pro-Implant | shoulder | | Kuehnast et | IA2-i1 |
| | | | | Foundation | | | al. 2018 | |
| 64 | HL013PA1 | IA2 | 3 | BEI | acne skin | NIH as | Tomida et al. | |
| | | | | Ressources | | part of | 2013; | |
| | | | | | | HMP | McDowell et | |
| | | | | | | | al. 2012 | |
| 65 | HL050PA3 | IA2 | 3 | BEI | skin | NIH as | Tomida et al. | |
| | | | | Ressources | | part of | 2013; | |
| | | | | | | HMP | McDowell et | |
| | | | | | | | al. 2012 | |
| 66 | HL025PA2 | IA2 | 3 | BEI | skin | NIH as | Tomida et al. | |
| | | | | Ressources | | part of | 2013; | |
| | | | | | | HMP | McDowell et | |
| | | | | | | | al. 2012 | |
| 67 | HL027PA1 | IA2 | 3 | BEI | skin | NIH as | Tomida et al. | |
| | | | | Ressources | | part of | 2013; | |
| | | | | | | HMP | McDowell et | |
| | | | | | | | al. 2012 | |
| 68 | HL002PA1 | IA2 | 3 | BEI | acne skin | NIH as | Tomida et al. | |
| | | | | Ressources | | part of | 2013; | |
| | | | | | | HMP | McDowell et | |
| | | | | | | | al. 2012 | |
| 69 | HL005PA4 | IA2 | 3 | BEI | skin | NIH as | Tomida et al. | |
| | | | | Ressources | | part of | 2013; | |
| | | | | | | HMP | McDowell et | |
| | | | | | | | al. 2012 | |
| 70 | HL030PA2 | IA2 | 3 | BEI | skin | NIH as | Tomida et al. | |
| | | | | Ressources | | part of | 2013; | |
| | | | | | | HMP | McDowell et | |
| | | | | | | | al. 2012 | |
| 71 | HL037PA1 | IA2 | 3 | BEI | skin | NIH as | Tomida et al. | |
| | | | | Ressources | | part of | 2013; | |
| | | | | | | HMP | McDowell et | |
| | | | | | | | al. 2012 | |
| 72 | HL050PA1 | IA2 | 3 | BEI | skin | NIH as | Tomida et al. | |
| | | | | Ressources | | part of | 2013; | |
| | | | | | | HMP | McDowell et | |
| | | | | | | | al. 2012 | |
| 73 | HL063PA2 | IA2 | 3 | BEI | skin | NIH as | Tomida et al. | |
| | | | | Ressources | | part of | 2013; | |
| | | | | | | HMP | McDowell et | |
| | | | | | | | al. 2012 | |
| 74 | HL059PA1 | IA2 | 16 | BEI | skin | NIH as | Tomida et al. | |
| | | | | Ressources | | part of | 2013; | |
| | | | | | | HMP | McDowell et | |
| | | | | | | | al. 2012 | |
| 75 | HL059PA2 | IA2 | 16 | BEI | skin | NIH as | Tomida et al. | |
| | | | | Ressources | | part of | 2013; | |
| | | | | | | HMP | McDowell et | |
| | | | | | | | al. 2012 | |
| 76 | HL067PA1 | IA2 | 3 | BEI | acne skin | NIH as | Tomida et al. | |
| | | | | Ressources | | part of | 2013; | |
| | | | | | | HMP | McDowell et | |
| | | | | | | | al. 2012 | |
| 77 | P.acn10 | IA2 | 1 | Queen's | corneal | McDowell | McDowell et | |
| | | | | University | scrape | et al. 2012 | al. 2012 | |
| | | | | Belfast | | | | |
| 78 | A019-#1 | IA2 | | Origimm | acne skin | | Origimm | |
| | | | | study | | | study | |
| 79 | KPA | IB | 1 | DSMZ | anaerobic | Douglas et | Brüggemann | |
| | 171202# | | | | contami- | al. and | et al. 2004 | |
| | (DSK16379) | | | | nation | Gunter | | |
| | | | | | | 1946 | | |
| 80 | P. acnes 87 | IB | 5 | Queen's | | | Kuehnast et | |
| | | | | University | | | al. 2018 | |
| | | | | Belfast | | | | |
| 81 | W1392 | IB | | Queen's | dental | | Kuehnast et | |
| | | | | University | isolate | | al. 2018 | |
| | | | | Belfast | | | | |
| 82 | IAI 002 | IB | | Pro-Implant | hip | | Kuehnast et | IB-i1 |
| | | | | Foundation | endo- | | al. 2018 | |
| | | | | | prosthesis | | | |
| 83 | IAI 005 | IB | | Pro-Implant | Aggregate | | Kuehnast et | IB-i2 |
| | | | | Foundation | | | al. 2018 | |
| 84 | IAI 015 | IB | | Pro-Implant | Osteo- | | Kuehnast et | IA1-i6 |
| | | | | Foundation | synthesis | | al. 2018 | |
| 85 | IAI 017 | IB | | Pro-Implant | knee | | Kuehnast et | IB-i5 |
| | | | | Foundation | endo- | | al. 2018 | |
| | | | | | prosthesis | | | |
| 86 | IAI 019 | IB | | Pro-Implant | Osteo- | | Kuehnast et | IB-i6 |
| | | | | Foundation | synthesis | | al. 2018 | |
| 87 | HL030PA1 | IB | 1 | BEI | Skin | NIH as | Tomida et al. | |
| | | | | Ressources | | part of | 2013; | |
| | | | | | | HMP | McDowell et | |
| | | | | | | | al. 2012 | |
| 88 | IAI031 | IB | | Pro-Implant | shoulder | | Origimm | |
| | | | | Foundation | implant | | study | |
| 89 | IAI034 | IB | | Pro-Implant | hip | | Origimm | |
| | | | | Foundation | endo- | | study | |
| | | | | | prosthesis | | | |
| 90 | IAI035 | IB | | Pro-Implant | Osteo- | | Origimm | |
| | | | | Foundation | synthesis | | study | |
| 91 | IAI038 | IB | | Pro-Implant | aggregate | | Origimm | |
| | | | | Foundation | | | study | |
| 92 | IAI040 | IB | | Pro-Implant | catheter | | Origimm | |
| | | | | Foundation | | | study | |
| 93 | IAI042 | IB | | Pro-Implant | Im- | | Origimm | |
| | | | | Foundation | plantable | | study | |
| | | | | | Cardio- | | | |
| | | | | | verter | | | |
| | | | | | Defib- | | | |
| | | | | | rillator | | | |
| | | | | | (ICD) | | | |
| 94 | IAI045 | IB | | Pro-Implant | loop | | Origimm | |
| | | | | Foundation | recorder | | study | |
| 95 | PV66 | IC | 5 | Queen's | acne skin | Barnard et | Barnard et al. | |
| | | | | University | | al. 2015 | 2015 | |
| | | | | Belfast | | | | |
| 96 | Hung.#2 | IC | | University | | | | IC-i2 |
| | | | | of Szeged | | | | |
| 97 | IAI 008 | IC | | Pro-Implant | loop | | Kuehnast et | IC-i1 |
| | | | | Foundation | recorder | | al. 2018 | |
| 98 | IAI 012 | II | | Pro-Implant | Screws | | Kuehnast et | II-i1 |
| | | | | Foundation | | | al. 2018 | |
| 99 | H009 SK1 | II | 2 | Origimm | skin | | Kuehnast et | II-s1 |
| | | | | study | | | al. 2018 | |
| 100 | HL001PA1 | II | 2 | BEI | skin | NIH as | Tomida et al. | |
| | | | | Ressources | | part of | 2013; | |
| | | | | | | HMP | McDowell et | |
| | | | | | | | al. 2012 | |
| 101 | HL050PA2 | II | 1 | BEI | skin | NIH as | Tomida et al. | |
| | | | | Ressources | | part of | 2013; | |
| | | | | | | HMP | McDowell et | |
| | | | | | | | al. 2012 | |
| 102 | HL060PA1 | II | 2 | BEI | acne skin | NIH as | Tomida et al. | |
| | | | | Ressources | | part of | 2013; | |
| | | | | | | HMP | McDowell et | |
| | | | | | | | al. 2012 | |
| 103 | HL110PA3 | II | 6 | BEI | acne skin | NIH as | Tomida et al. | |
| | | | | Ressources | | part of | 2013; | |
| | | | | | | HMP | McDowell et | |
| | | | | | | | al. 2012 | |
| 104 | HL110PA4 | II | 6 | BEI | acne skin | NIH as | Tomida et al. | |
| | | | | Ressources | | part of | 2013; | |
| | | | | | | HMP | McDowell et | |
| | | | | | | | al. 2012 | |
| 105 | HL082PA2 | II | 2 | BEI | acne skin | NIH as | Tomida et al. | |
| | | | | Ressources | | part of | 2013; | |
| | | | | | | HMP | McDowell et | |
| | | | | | | | al. 2012 | |
| 106 | IAI041 | II | | Pro-Implant | lumbar | | Origimm | |
| | | | | Foundation | spine | | study | |
| 107 | ATCC | II | | ATCC | Sub- | | Horvath et | |
| | 11828 | | | | cutaneous | | al., 2012 | |
| | | | | | abscess | | | |
| 108 | Asn12 | III | 9 | Queen's | spinal disk | McDowell | Barnard et al. | |
| | | | | University | | et al. 2012 | 2015 | |
| | | | | Belfast | | | | |
| 109 | 20C | III | 9 | Queen's | skin | McDowell | Kuehnast et | III-s1 |
| | | | | University | | et al. 2012 | al. 2018 | |
| | | | | Belfast | | | | |
| 110 | IAI 010 | III | | Pro-Implant | hip endo- | | Kuehnast et | III-i1 |
| | | | | Foundation | prosthesis | | al. 2018 | |

*alternative name is the strain identification number used in the publication by Kuehnast et al. 2018
#the first P. acnes strain completely sequenced; used as a reference in many published studies BEI Resources refers to the strains obtained from NIAID, NIH as part of the Human Microbiome Project: Propionibacterium acnes..
We thank Professor Sheila Patrick and Andrew McDowell, Queen's University, Belfast; Prof. Istvan Nagy at Institute of Biochemistry, Biological Research Centre of the Hungarian Academy of Sciences, Szeged; and Dr. Andrej Trampuz at Pro-Implant Foundation, Berlin; for access to the additional C. acnes strains used in this study.

### BIBLIOGRAPHY

Achermann et al., Clin. Microbiol. Rev. 27 (2014), 419-440
Achermann Y, Tran B, Kang M et al. Immunoproteomic identification of in vivo-produced Propionibacterium acnes proteins in a rabbit biofilm infection model. Clin Vaccine Immunol 2015; 22:467-76.
Alexeyev O A, Zouboulis C C. Shooting at skin Propionibacterium acnes: To be or not to be on target. J Invest Dermatol 2013; 133:2292-4.
Allhorn M, Arve S, Brüggemann H et al. A novel enzyme with antioxidant capacity produced by the ubiquitous skin colonizer Propionibacterium acnes. Nat Publ Gr 2016:1-12; Sci. Rep. 6 (2016): 36412.
Athey et al., BMC Bioinformatics 18 (2017), 391.
Barnard E, Liu J, Yankova E et al. Strains of the Propionibacterium acnes type III lineage are associated with the skin condition progressive macular hypomelanosis. Sci Rep 2016; 6:1-9.
Barnard E, Nagy I, Hunyadkürti J et al. Multiplex touchdown PCR for rapid typing of the opportunistic pathogen Propionibacterium acnes. J Clin Microbiol 2015; 53:1149-55.
Bek-Thomsen M, Lomholt H B, Scavenius C et al. Proteome analysis of human sebaceous follicle infundibula extracted from healthy and acne-affected skin. PLoS One 2014; 9:e107908.
Borelli et al., Acta Derm. Venerol. 86 (2006), 316-319
Bruggemann H, Henne A, Hoster F et al. The complete genome sequence of Propionibacterium acnes, a commensal of human skin. Science (80-) 2004; 305:671-3.
Brüggemann, Skin: Acne and P. acnes Genomics, in Timmis (ed.), “Handbook of Hydrocarbon and Lipid Microbiology”, DOI 10.1007/978-3-540-77587-4_244; Springer-Verlag Berlin Heidelberg, 2010, pages 3215 to 3225
Brüggemann H. Skin: Cutibacterium (formerly Propionibacterium) acnes and Acne Vulgaris. Heal Consequences Microb Interact with Hydrocarb Oils, Lipids 2019:1-20.
Brzuszkiewicz E, Weiner J, Wollherr A et al. Comparative genomics and transcriptomics of Propionibacterium acnes. PLoS One 2011; 6:e21581.
Burkhart C G, Burkhart C N. Expanding the microcomedone theory and acne therapeutics: Propionibacterium acnes biofilm produces biological glue that holds corneocytes together to form plug. J Am Acad Dermatol 2007; 57:722-4.
Cao et al., J. Chem. Inf. Model. 59 (2019). 1508-1514
Capoor et al., Eur. Spine J. 28 (2019). 2952-2971
Chen et al., Comput. Biol. 16 (2020), e1008543
Coenye T, Peeters E, Nelis H J. Biofilm formation by Propionibacterium acnes is associated with increased resistance to antimicrobial agents and increased production of putative virulence factors. Res Microbiol 2007; 158:386-92.
Colussi et al., Nat. 519 (2015), 110-113.
Cong et al., Arch. Dermatol. Res. 311 (2019), 337-349
Contassot E. Vaccinating against Acne: Benefits and Potential Pitfalls. 2018:1-3.
Crommelin et al., J. Phar. Sci. 110 (2021), 627-634,
Dagnelie M A, Khammari A, Dréno B et al. Cutibacterium acnes molecular typing: time to standardize the method. Clin Microbiol Infect 2018; 24:1149-55.
Dekio I, Rajendram D, Morita E et al. Genetic diversity of Propionibacterium acnes strains isolated from human skin in Japan and comparison with their distribution in Europe. J Med Microbiol 2012; 61:622-30.
Douglas H C, Gunter S E. The taxonomic position of Corynebacterium acnes. J Bacteriol 1946; 52:15-23.
Dutra et al., Front. Pharmacol. 5 (2014), Art. 115
Eady E A, Layton A M. A distinct acne microbiome: Fact or fiction? J Invest Dermatol 2013; 133:2294-5.
Engelke L, Winter G, Hook S et al. Recent insights into cutaneous immunization: How to vaccinate via the skin. Vaccine 2015; 33:4663-74.
Fischer N, Mak T N, Shinohara D B et al. Deciphering the Intracellular Fate of Propionibacterium acnes in Macrophages. Biomed Res Int 2013; 2013:1-11.
Fischetti V A. Streptococcal M protein: Molecular design and biological behavior. Clin Microbiol Rev 1989; 2:285-314.
Fitz-Gibbon S, Tomida S, Chiu B H et al. Propionibacterium acnes strain populations in the human skin microbiome associated with acne. J Invest Dermatol 2013; 133:2152-60.
Forster et al., PLoS Med 17 (2020), pmed.1003024
Gilchrist T. A bacteriological and microscopical study of over three hundred vesicular and pustular lesions of the skin, with a research upon the etiology of Acne vulgaris. Johns Hopkins Hosp Rep 1900; 9:409-30.
Giuliani M M, Adu-Bobie J, Comanducci M et al. A universal vaccine for serogroup B meningococcus. Proc Natl Acad Sci USA 2006; 103:10834-9.
Gordon S. Phagocytosis: An Immunobiologic Process. Immunity 2016; 44:463-75.
Grange P A, Raingeaud J, Morelle W et al. Characterization of a Propionibacterium acnes Surface Protein as a Fibrinogen-Binding Protein. Sci Rep 2017; 7:1-14.
Holland C, Mak T N, Zimny-Arndt U et al. Proteomic identification of secreted proteins of Propionibacterium acnes. BMC Microbiol 2010; 10:230.
Holmberg A, Lood R, Mörgelin M et al. Biofilm formation by Propionibacterium acnes is a characteristic of invasive isolates. Clin Microbiol Infect 2009; 15:787-95.
Holtkamp et al., Blood 108 (2006), 4009-4017.
Horstick et al., NAR 43 (2015), e48.
Horwath et al., J Bacteriol. 194 (2012): 202-203.
Jahns A C, Lundskog B, Ganceviciene R et al. An increased incidence of Propionibacterium acnes biofilms in acne vulgaris: A case-control study. Br J Dermatol 2012; 167:50-8.
Jalkanen et al., Semin. Cell Dev. Biol. 0 (2014), 24-32.
Keshari S, Kumar M, Balasubramaniam A et al. Prospects of acne vaccines targeting secreted virulence factors of Cutibacterium acnes. Expert Rev Vaccines 2019; 18:433-7.
Kim et al., Nat. Struct. Mol. Biol. 27 (2020), 581-588.
Knittelfelder R, Riemer A B, Jensen-Jarolim E. Mimotope vaccination—From allergy to cancer. Expert Opin Biol Ther 2009; 9:493-506.
Kuehnast T, Cakar F, Weinhaupl T et al. Comparative analyses of biofilm formation among different Cutibacterium acnes isolates. Int J Med Microbiol 2018; 308:1027-35.
Lee, Byun, Kim. Potential Role of the Microbiome in Acne: A Comprehensive Review. J Clin Med 2019; 8:987.
Liu J, Cheng A, Bangayan N J et al. Draft genome sequences of Propionibacterium acnes type strain ATCC6919 and antibiotic-resistant strain HL411PA1. Genome Announc 2014; 2:e00740-14.
Liu P F, Nakatsuji T, Zhu W et al. Passive immunoprotection targeting a secreted CAMP factor of Propionibacterium acnes as a novel immunotherapeutic for acne vulgaris. Vaccine 2011; 29:3230-8.
Lodes M J, Secrist H, Benson D R et al. Variable expression of immunoreactive surface proteins of Propionibacterium acnes. Microbiology 2006; 152:3667-81.
Lomholt H B, Kilian M. Population genetic analysis of Propionibacterium acnes identifies a subpopulation and epidemic clones associated with acne. PLoS One 2010; 5:el2277.
Lovecková Y, Havlíková I. A microbiological approach to acne vulgaris. Biomed Pap Med Fac Univ Palacky Olomouc Czech Repub 2002; 146:29-32.
Macdougall I C, Rossert J, Casadevall N et al. A peptide-based erythropoietin-receptor agonist for pure red-cell aplasia. N Engl J Med 2009; 361:1848-55.
Malonis et al., Chem. Rev. 120 (2020), 3210-3229
Mauger et al., PNAS 116 (2019), 24075-24083.
Mayslich et al., Microorg. 9 (2021), 303
McDowell et al., J. Clin. Microbiol. 43 (2005), 326-334
McDowell A, Barnard E, Nagy I et al. An expanded multilocus sequence typing scheme for Propionibacterium acnes: Investigation of “pathogenic”, “commensal” and antibiotic resistant strains. PLoS One 2012; 7:e41480.
McDowell A, Gao A, Barnard E et al. A novel multilocus sequence typing scheme for the opportunistic pathogen Propionibacterium acnes and characterization of type I cell surfaceassociated antigens. Microbiology 2011; 157:1990-2003.
McDowell et al., BioMed Res. Int. 2015, ID 493564.
McLaughlin J, Watterson S, Layton A M et al. Propionibacterium acnes and Acne Vulgaris: New Insights from the Integration of Population Genetic, Multi-Omic, Biochemical and Host-Microbe Studies. Microorganisms 2019; 7:128.
Miah, Biotechnol. 1 (2002), 21-27.
Morris G E. Epitope mapping. Methods Mol Biol 2005; 295:255-68.
Myszka et al., Biophys. J. 75 (1998), 583-594
Nakatsuji T, Liu Y T, Huang C P et al. Vaccination targeting a surface sialidase of P. acnes: Implication for new treatment of acne vulgaris. PLoS One 2008; 3:e1551.
Nicholson et al., Tr. Cell Biol. 29 (2019), 191-200.
O'Neill A M, Gallo R L. Host-microbiome interactions and recent progress into understanding the biology of acne vulgaris. Microbiome 2018; 6:1-16.
Pardi et al., NRDD 17 (2018), 261-279
Petronelli. Dermatology Times February 2021, 22;: “Next-Gen Therapies Advance Acne Treatment”.
Portillo et al., BioMed Res. Int. 2013, ID 804391.
Prausitz et al., Curr. Top. Microbiol. Immunol. 333 (2009), 369-393
Sadhasivam S, Sinha M, Saini S et al. Heterogeneity and antibiotic resistance in Propionibacterium acnes isolates and its therapeutic implications: blurring the lines between commensal and pathogenic phylotypes. Dermatol Ther 2016; 29:451-4.
Sayanjali et al., Int. J. Med. Microbiol. 306 (2016). 517-528
Sasaki et al., PNAS 97 (2000), 1512-1515.
Schaller et al., Br. J. Dermatol. 153 (2005), 66-71
Scholz C F P, Jensen A, Lomholt H B et al. A novel high-resolution single locus sequence typing scheme for mixed populations of Propionibacterium acnes in vivo. PLoS One 2014; 9:e104199.
Shu et al., Curr. Med. Chem. 20 (2013), 562-568.
Schupp et al., BMC Pulm. Med. 15 (2015), 75
Smith G P, Petrenko V A. Phage display. Chem Rev 1997; 97:391-410.
Taylor P W. Bactericidal and bacteriolytic activity of serum against gram-negative bacteria. Microbiol Rev 1983; 47:46-83.
Theodorou et al., JBC 295 (2020), 5519-5532
Thess et al., Mol. Ther. 23 (2015), 1456-1464.
Tomida S, Nguyen L, Chiu B et al. Pan-Genome and Comparative Genome Analyses of. Am Soc Microbiol 2013; 4:1-11.
VanBlargan L A, Goo L, Pierson T C, Deconstructing the Antiviral Neutralizing-Antibody Response: Implications for Vaccine Development and Immunity. Microbiol Mol Biol Rev 2016; 80:989-1010.
Verma et al., Nat. Commun. (2019), 10-5774-s41467_019_Article_13810
Vörös A, Horvith B, Hunyadkurti J et al. Complete genome sequences of three Propionibacterium acnes isolates from the type ia 2 cluster. J Bacteriol 2012; 194:1621-2.
Wang Y, Hata T R, Tong Y L et al. The Anti-Inflammatory Activities of Propionibacterium acnes CAMP Factor-Targeted Acne Vaccines. J Invest Dermatol 2018:1-10.
Webster G F, Leyden J J, Musson R A et al. Susceptibility of Propionibacterium acnes to killing and degradation by human neutrophils and monocytes in vitro. Infect Immun 1985; 49:116-21.
Wingfield, Curr. Protoc. Protein Sci. 88 (2018), 6.14.1-6.14.3
van Westen E, Rodenburg G D, van Gils E J M et al. Levels and functionality of antibodies after pneumococcal conjugate vaccine in schedules with different timing of the booster dose. Vaccine 2013; 31:5834-42.
Xiao et al, Biochemistry 49 (2010), 5588-5599.
Yang et al., J. Mol. Cell Biol. 11 (2019), 911-919.
Yu Y, Champer J, Agak G W et al. Different Propionibacterium acnes Phylotypes Induce Distinct Immune Responses and Express Unique Surface and Secreted Proteomes. J Invest Dermatol 2016; 136:2221-8.
Yu Y, Champer J, Kim J. Analysis of the surface, secreted, and intracellular proteome of Propionibacterium acnes. EuPA Open Proteomics 2015; 9:1-7.
Zaenglein A L, Pathy A L, Schlosser B J et al. Guidelines of care for the management of acne vulgaris. J Am Acad Dermatol 2016; 74:945-73.e33.
Zaluga E. Skin reactions to antigens of Propionibacterium acnes in patients with acne vulgaris treated with autovaccine. Ann Acad Medicae Stetin (Ann Acad Med Stetin) 1998; 44:65-85.
Zhang et al., Front. Immunol. 10 (2019), 594.
Zouboulis C C, Dessinioti C, Tsatsou F et al. Anti-acne drugs in phase 1 and 2 clinical trials. Expert Opin Investig Drugs 2017; 26:813-23.

Sequences
Uniprot Q6A5X9
(SEQ ID NO: 64)
MLSLLRRLLR WSDQSQQSPP PPPPQAEASS NRPRSVAQAA
IATDGKGIID KDCRDAVIND AKLRAAIAGA LVKAGESSAD
AVALAPRIAK EMAKEGVLLI NHHKLKALIG AQLGLLTDAK
IQRAAAAVDL GIKATLAATI IPNALHSAAF KDAVVANLVA
AGVDKKLAKA TAVAIAATAL NPALGPIAKT EAIKAEIAAQ
AALLVGRGVH LKKAAIEHII GRSFDAAVAT AIVSSPILNA
RIVTHLVRAG IDKSLAVQIA PRIIDRLAKE PLLALNTAKL
MKNITRQIVD VITADKAIKT AEQLEKELPA LDDLVKKACS
CPKPTPTPTP TPTPTPKPTP TPTPKPTPTP KPKPTPAPAP
TSGATSDEST SRSGGHSQGG SGTHYIHHGV APVLTHSSDL
PSTGF
Uniprot Q6A5P9
(SEQ ID NO: 65)
MIIFVRVGNR HMRKRRAVLV VAFQLVNRRL AITKEPTPRR
NDASNKNHCG SCDSNCLNRL TISYHPLPAN AASNGNSSIT
QSAAFSPRAT TKISEDCRKA IINDLKLRGA IVGALVKAGL
SAADAAALAP RIAAEMAAEG TLTINHHRLK VLVASQLGLV
ADAAVQHAAA AIDLSFKAIL GASIIPNALG SAAFKNAVIA
NLVAAGIDKH LARATAVAIV ATALNPALGP IAKFELIKAE
IAAQAALLIR RGVHLQKAAI EHVIGRAFDA AVATAIISSP
ILSARIVTHL VRAGIDKSIA ISLAPHIVKR LAKEPLLAFN
TAKLVKDIAR QIVDIRNTQE AIAVYKQLKA ELPTLDGLVQ
KACTPEPTPT PTPTPTPTPT PAPTPTPAPT PTPAPTPAPT
PTPAPTPTPT PTPTPTPTPT HGATTTTPIS RTTDRHNLGS
HHTRIAAPAL IHAKALPATG TGA
Uniprot Q6A9N1
(SEQ ID NO: 66)
MTTSAMRKIV ASVLGAILAL TGVLITPAAW AAGPTVTVIP
VGREGGDITI SGKGFSTTGF GVYVAVAPAS VPEFYGNSDK
FYGYDPSKDT TESPSTIWVY TPSQKAIGSR FAQGRPMNND
GSFTITMKAP PFEQGKDFVV LTTKAHGVGK TDHSDDTRTP
VTYREATPAP TGPKTPIAPS KQPSKQAAPS KQVKPSKQAG
PNKQSTTPQQ KTAEHRSQTP AAHRTMTKQV CTIGASKVTS
GSLTWGIRTS FTSYLRGPIA NGSWKLSGGA NWNGSAFTFP
LTSGSEDPAT KSGSLKYSGS VHMTGHHGIL DMTLAEPSLQ
IKGSTGHLYL DVKSSSMDGK KTNYGRVDFA TFGVSVSGNA
AIKGSPVKLT ATGAKAFAGF YRAGEPMNPL STNLTLSAEK
VCHNVTVDAV TGKVIGDDSG KGAGRGLPVT GAEGPSSDEI
DLGIVGGLAL TAVVSTVVVC RRYAARI
P002
(SEQ ID NO: 1)
MHAVEPTTTI SATSTHELSA SDARNSIQLL NAHIATLQSV
QKSVPGSDYS DQIRDLLKAA FDLRGLIETL AHGGIPFYDP
STIMPRIKLV ATTIDTIHTA TTTLQNKVRP AHVELGLEVT
KAVLLTANPA STAKELDAEG AALKARLEKV SQYPDLTPND
VATVYVRTNF SKTIWQVRAN RDRYILGHKS AAVYKTLNHA
ITKAVGVRLN PKTTVGNIQA ARTELLAAYQ TAFNSPDVKK
AAHHHHHH
P005
(SEQ ID NO: 2)
MTVKVGINGF GRIGRNFFRA LAEQGADLEV VAVNDLTDTK
TLAHLLKYDS IMGRFSGEVS YDDDSITVDG KTIKVLAERN
PADLPWKELG AEIVVESTGL FTDGEKAKAH FEAGAKKVII
SAPGKNVDGT FVMGVNDGDY DNAKHNIISN ASCTTNCLAP
LAKVLNDAFG IERGIMTTVH AYTGDQRLQD APHKDLRRAR
AAALNMIPTK TGAAQAVALV LPELKGKFDG LAVRVPTPTG
SLTDLTFQTS KETTVEEVQA AVKKAAEGPL KGILAYTEDP
IVSKDIEGDP HSSIFDATET KAIGNLVKVL SWYDNEWGYS
NRLVDLTKLV ASKLAHHHHH H
P018
(SEQ ID NO: 3)
MSSELVDTTE MYLRTFYELL EEGVELRRAR IVERLHQSGP
TVSQTVARME RDGLVVVESD RSISLTQEGH DVAQTVMRKH
RLAECLLTQI IGLRPDLVHD EACRWEHVIS GEVEKRLTGL
LENPDVSPYG CPLPPEQAGC RPDGSARFRD DSKPLDEVIA
ETGCPVSVTV TRLSEFFQAT EGNLADVYAA GLLPGKSVEV
DDDADGIRLT GPSGSIVVDP EILSGLFVAQ NSHHHHHH
P022
(SEQ ID NO: 4)
MHHHHHHLSL LRRLLRWSDQ SQQSPPPPPP QAEASSNRPR
SVAQAAIATD GKGIIDKDCR DAVINDAKLR AAIAGALVKA
GESSADAVAL APRIAKEMAK EGVLLINHHK LKALIGAQLG
LLTDAKIQRA AAAVDLGIKA TLAATIIPNA LHSAAFKDAV
VANLVAAGVD KKLAKATAVA IAATALNPAL GPIAKTEAIK
AEIAAQAALL VGRGVHLKKA AIEHIIGRSF DAAVATAIVS
SPILNARIVT HLVRAGIDKS LAVQIAPRII DRLAKEPLLA
LNTAKLMKNI TRQIVDVITA DKAIKTAEQL EKELPALDDL
VKKACSCPKP TPTPTPTPTP TPKPTPTPTP KPTPTPKPKP
TPAPAPTSGA TSDESTSRSG GHSQGGSGTH YIHHGVAPVL
THSSDLPSTG F
P022-V1
(SEQ ID NO: 5)
MHHHHHHDSQ IRGEAMRRSR IAVAAATATA LVGSIAAITP
SPQAEASSNR PRSVAQAAIA TDGKGIINKD CRDAVINDAK
LRAAIAGALV KAGESSADAV ALAPRIAREM AKEGVLLINH
HKLKALIGAQ VGLLTDAKIQ RAAAAVDLGI KATLAATIIP
NALHSAAFKD AVVANLVAAG VDKKLAKATA VAIAATALNP
ALGPIAKTEA IKAEIGAQAA LLVSRGVHLK KAAIEHIIGR
SFDAAVATAI VSSPILNARI VTHLVRAGID KSLAVQIAPR
IIDRLAKEPL LALNTAKLMK NITRQIVDVI TADKAIKTAE
QLEKELPALD DLVKKACSCP KPTPTPTPTP TPTPTPKPTP
TPTPTPTPTP KPKPTPAPAP TSGATSDEST SRSGGHSQGG
SGTHYIHHGV APVLTHSSDL PSTGF
P022-V2
(SEQ ID NO: 6)
MHHHHHHSSN RPRSVAQAAI ATDGKGIIDK DCRDAVINDA
KLRAAIAGAL VKAGFSSADA VALAPRIAKE MAKEGVLLIN
HHKLKALIGA QLGLLTDAKI QRAAAAVDLG IKATLAATII
PNALHSAAFK DAVVANLVAA GVDKKLAKAT AVAIAATALN
PALGPIAKTE AIKAEIAAQA ALLVGRGVHL KKAAIEHIIG
RSFDAAVATA IVSSPILNAR IVTHLVRAGI DKSLAVQIAP
RIIDRLAKEP LLALNTAKLM KNITRQIVDV ITADKAIKTA
EQLEKELPAL DDLVKKACSC PKPTPTPTPT PTPTPKPTPT
PTPKPTPTPK PKPTPAPAPT SGATSDESTS RSGGHSQGGS
GTHYIHHGVA PVLTHSSDLP STGF
P022-V3
(SEQ ID NO: 7)
MSSNRPRSVA QAAIATDGKG IIDKDCRDAV INDAKLRAAI
AGALVKAGES SADAVALAPR IAKEMAKEGV LLINHHKLKA
LIGAQLGLLT DAKIQRAAAA VDLGIKATLA ATHIPNALHS
AAFKDAVVAN LVAAGVDKKL AKATAVAIAA TALNPALGPI
AKTEAIKAEI AAQAALLVGR GVHLKKAAIE HIIGRSFDAA
VATAIVSSPI LNARIVTHLV RAGIDKSLAV QIAPRIIDRL
AKEPLLALNT AKLMKNITRQ IVDVITADKA IKTAEQLEKE
LPALDDLVKK ACSCPKPTPT PTPTPTPTPK PTPTPTPKPT
PTPKPKPTPA PAPTSGATSD ESTSRSGGHS QGGSGTHYIH
HGVAPVLTHS SDLPSTGF
P027
(SEQ ID NO: 8)
MITKFPTPRR NDASNKNHCG SCDSNCLNRL TISYHPLPAN
AASNGNSSIT QSAAFSPRAT TKISEDCRKA IINDLKLRGA
IVGALVKAGL SAADAAALAP RIAAEMAAEG TLTINHHRLK
VLVASQLGLV ADAAVQHAAA AIDLSFKAIL GASIIPNALG
SAAFKNAVIA NLVAAGIDKH LARATAVAIV ATALNPALGP
IAKFELIKAE IAAQAALLIR RGVHLQKAAI EHVIGRAFDA
AVATAIISSP ILSARIVTHL VRAGIDKSIA ISLAPHIVKR
LAKEPLLAFN TAKLVKDIAR QIVDIRNTQE AIAVYKQLKA
ELPTLDGLVQ KACTPEPTPT PTPTPTPTPT PAPTPTPAPT
PTPAPTPAPT PTPAPTPTPT PTPTPTPTPT HGATTTTPIS
RTTDRHNLGS HHTRIAAPAL IHAKALPATG TGAHHHHHH
P027-V1
(SEQ ID NO: 9)
MIIFVRVGNR HMRKRRAVLV VAFQLVNRRL AITKFPTPRR
NDASNKNHCG SCDSNCLNRL TISYHPLPAN AASNGNSSIT
QSAAFSPRAT TKISEDCRKA IINDLKLRGA IVGALVKAGL
SAADAAALAP RIAAEMAAEG TLTINHHRLK VLVASQLGLV
ADAAVQHAAA AIDLSFKAIL GASIIPNALG SAAFKNAVIA
NLVAAGIDKH LARATAVAIV ATALNPALGP IAKFELIKAE
IAAQAALLIR RGVHLQKAAI EHVIGRAFDA AVATAIISSP
ILSARIVTHL VRAGIDKSIA ISLAPHIVKR LAKEPLLAFN
TAKLVKDIAR QIVDIRNTQE AIAVYKQLKA ELPTLDGLVQ
KACTPEPTPT PTPTPTPTPT PAPTPTPAPT PTPAPTPAPT
PTPAPTPTPT PTPTPTPTPT HGATTTTPIS RTTDRHNLGS
HHTRIAAPAL IHAKALPATG TGAHHHHHH
P027-V2
(SEQ ID NO: 10)
MHHHHHHLSP SFQPLGETMR RTRITVAAVT ATALIGSLSA
ITPLPANAAS NGNSSITQSA AFSPRATTKI SEDCRKAIIN
DLKLRGAIVG ALVKAGLSAA DAAALAPRIA AEMAAEGTLT
INHHRLKVLV ASQLGLVADA AVQHAAAAID LSFKAILGAS
IIPNALGSAA FKNAVIANLV AAGIDKHLAR ATAVAIVATA
LNPALGPIAK FELIKAEIAA QAALLIRRGV HLQKAAIEHV
IGRAFDAAVA TAIISSPILS ARIVTHLVRA GIDKSIAISL
APHIVKRLAK EPLLAFNTAK LVKDIARQIV DIRNTQEAIA
VYKQLKAELP TLDGLVQKAC TPEPTPTPTP TPTPTPTPTP
TPTPTPTPTP TPTPTPAPTP APTPAPTPTP TPTPAPTPTP
TPTPTPTPTP TPTPTPTPTH GATTTTPISR TTDRHNLGSH
HTRIAAPALI HAKALPATGT GA
P027-V3
(SEQ ID NO: 11)
MHHHHHHASN GNSSITQSAA FSPRATTKIS EDCRKAIIND
LKLRGAIVGA LVKAGLSAAD AAALAPRIAA EMAAEGTLTI
NHHRLKVLVA SQLGLVADAA VQHAAAAIDL SFKAILGASI
IPNALGSAAF KNAVIANLVA AGIDKHLARA TAVAIVATAL
NPALGPIAKF ELIKAEIAAQ AALLIRRGVH LQKAAIEHVI
GRAFDAAVAT AIISSPILSA RIVTHLVRAG IDKSIAISLA
PHIVKRLAKE PLLAFNTAKL VKDIARQIVD IRNTQEAIAV
YKQLKAELPT LDGLVQKACT PEPTPTPTPT PTPTPTPAPT
PTPAPTPTPA PTPAPTPTPA PTPTPTPTPT PTPTPTHGAT
TTTPISRTTD RHNLGSHHTR IAAPALIHAK ALPATGTGA
P027-V4
(SEQ ID NO: 12)
MASNGNSSIT QSAAFSPRAT TKISEDCRKA IINDLKLRGA
IVGALVKAGL SAADAAALAP RIAAEMAAEG TLTINHHRLK
VLVASQLGLV ADAAVQHAAA AIDLSFKAIL GASIIPNALG
SAAFKNAVIA NLVAAGIDKH LARATAVAIV ATALNPALGP
IAKFELIKAE IAAQAALLIR RGVHLQKAAI EHVIGRAFDA
AVATAIISSP ILSARIVTHL VRAGIDKSIA ISLAPHIVKR
LAKEPLLAFN TAKLVKDIAR QIVDIRNTQE AIAVYKQLKA
ELPTLDGLVQ KACTPEPTPT PTPTPTPTPT PAPTPTPAPT
PTPAPTPAPT PTPAPTPTPT PTPTPTPTPT HGATTTTPIS
RTTDRHNLGS HHTRIAAPAL IHAKALPATG TGA
P028
(SEQ ID NO: 13)
MHHHHHHAGP TVTVIPVGRE GGDITISGKG FSTTGFGVYV
AVAPASVPEF YGNSDKFYGY DPSKDTTESP STIWVYTPSQ
KAIGSRFAQG RPMNNDGSFT ITMKAPPFEQ GKDFVVLTTK
AHGVGKTDHS DDTRTPVTYR EATPAPTGPK TPIAPSKQPS
KQAAPSKQVK PSKQAGPNKQ STTPQQKTAE HRSQTPAAHR
TMTKQVCTIG ASKVTSGSLT WGIRTSFTSY LRGPIANGSW
KLSGGANWNG SAFTFPLTSG SFDPATKSGS LKYSGSVHMT
GHHGILDMTL AEPSLQIKGS TGHLYLDVKS SSMDGKKTNY
GRVDFATFGV SVSGNAAIKG SPVKLTATGA KAFAGFYRAG
EPMNPLSTNL TLSAEKVCHN VTVDAVTGKV IGDDSGKGAG
RGLPVTGAEG PSSDEIDLGI VGGLALTAVV STVVVCRRYA
ARI
P028-V1
(SEQ ID NO: 14)
MAGPTVTVIP VGREGGDITI SGKGFSTTGF GVYVAVAPAS
VPEFYGNSDK FYGYDPSKDT TESPSTIWVY TPSQKAIGSR
FAQGRPMNND GSFTITMKAP PFEQGKDFVV LTTKAHGVGK
TDHSDDTRTP VTYREATPAP TGPKTPIAPS KQPSKQAAPS
KQVKPSKQAG PNKQSTTPQQ KTAEHRSQTP AAHRTMTKQV
CTIGASKVTS GSLTWGIRTS FTSYLRGPIA NGSWKLSGGA
NWNGSAFTFP LTSGSFDPAT KSGSLKYSGS VHMTGHHGIL
DMTLAEPSLQ IKGSTGHLYL DVKSSSMDGK KTNYGRVDFA
TFGVSVSGNA AIKGSPVKLT ATGAKAFAGF YRAGEPMNPL
STNLTLSAEK VCHNVTVDAV TGKVIGDDSG KGAGRGLPVT
P028-V2
(SEQ ID NO: 15)
MAGPTVTVIP VGREGGDITI SGKGFSTTGF GVYVAVAPAS
VPEFYGNSDK FYGYDPSKDT TESPSTIWVY TPSQKAIGSR
FAQGRPMNND GSFTITMKAP PFEQGKDFVV LTTKAHGVGK
TDHSDDTRTP VTYREATPAP TGPKTPIAPS KQPSKQAAPS
KQVKPSKQAG PNKQSTTPQQ KTAEHRSQTP AAHRTMTKQV
CTIGASKVTS GSLTWGIRTS FTSYLRGPIA NGSWKLSGGA
NWNGSAFTFP LTSGSFDPAT KSGSLKYSGS VHMTGHHGIL
DMTLAEPSLQ IKGSTGHLYL DVKSSSMDGK KTNYGRVDFA
TFGVSVSGNA AIKGSPVKLT ATGAKAFAGF YRAGEPMNPL
STNLTLSAEK VCHNVTVDAV TGKVIGDDSG KGAGRGLPVT
GAEGPSSDEI DLGIVGGLAL TAVVSTVVVC RRYAARI
P028-V3
(SEQ ID NO: 16)
MAGPTVTVIP VGREGGDITI SGKGFSTTGF GVYVAVAPAS
VPEFYGNSDK FYGYDPSKDT TESPSTIWVY TPSQKAIGSR
FAQGRPMNND GSFTITMKAP PFEQGKDFVV LTTKAHGVGK
TDHSDDTRTP VTYREATPAP TGPSGSLTWG IRTSFTSYLR
GPIANGSWKL SGGANWNGSA FTFPLTSGSF DPATKSGSLK
YSGSVHMTGH HGILDMTLAE PSLQIKGSTG HLYLDVKSSS
MDGKKTNYGR VDFATFGVSV SGNAAIKGSP VKLTATGAKA
FAGFYRAGEP MNPLS
P028-V4
(SEQ ID NO: 17)
MAGPTVTVIP VGREGGDITI SGKGESTTGF GVYVAVAPAS
VPEFYGNSDK FYGYDPSKDT TESPSTIWVY TPSQKAIGSR
FAQGRPMNND GSFTITMKAP PFEQGKDFVV LTTKAHGVGK
TDHSDDTRTP VTYREATPAP TGPKTPIAPS KQPSKQAAPS
KQVKPSKQAG PNKQSTTPQQ KTAEHRSQTP AAHRTMTKQV
CTIGASKVTS GSLTWGIRTS FTSYLRGPIA NGSWKLSGGA
NWNGSAFTFP LTSGSFDPAT KSGSLKYSGS VHMTGHHGIL
DMTLAEPSLQ IKGSTGHLYL DVKSSSMDGK KTNYGRVDFA
TFGVSVSGNA AIKGSPVKLT ATGAKAFAGF YRAGEPMNPL
S
P028-V5
(SEQ ID NO: 18)
MAGPTVTVIP VGREGGDITI SGKGFSTTGF GVYVAVAPAS
VPEFYGNSDK FYGYDPSKDT TESPSTIWVY TPSQKAIGSR
FAQGRPMNND GSFTITMKAP PFEQGKDFVV LTTKAHGVGK
TDHSDDTRTP VTYREATPAP TGPKTPIAPS KQPSKQAAPS
KQVKPSKQAG PNKQSTTPQQ KTAEHRSQTP AAHRTMTKQV
STIGASKVTS GSLTWGIRTS FTSYLRGPIA NGSWKLSGGA
NWNGSAFTFP LTSGSFDPAT KSGSLKYSGS VHMTGHHGIL
DMTLAEPSLQ IKGSTGHLYL DVKSSSMDGK KTNYGRVDFA
TFGVSVSGNA AIKGSPVKLT ATGAKAFAGF YRAGEPMNPL
STNLTLSAEK VCHNVTVDAV TGKVIGDDSG KGAGRGLPVT
P028-V6
(SEQ ID NO: 19)
MAGPTVTVIP VGREGGDITI SGKGESTTGF GVYVAVAPAS
VPEFYGNSDK FYGYDPSKDT TESPSTIWVY TPSQKAIGSR
FAQGRPMNND GSFTITMKAP PFEQGKDFVV LTTKAHGVGK
TDHSDDTRTP VTYREATPAP TGPKTPIAPS KQPSKQAAPS
KQVKPSKQAG PNKQSTTPQQ KTAEHRSQTP AAHRTMTKQV
CTIGASKVTS GSLTWGIRTS FTSYLRGPIA NGSWKLSGGA
NWNGSAFTFP LTSGSFDPAT KSGSLKYSGS VHMTGHHGIL
DMTLAEPSLQ IKGSTGHLYL DVKSSSMDGK KTNYGRVDFA
TFGVSVSGNA AIKGSPVKLT ATGAKAFAGF YRAGEPMNPL
STNLTLSAEK VSHNVTVDAV TGKVIGDDSG KGAGRGLPVT
P028-V7
(SEQ ID NO: 20)
MAGPTVTVIP VGREGGDITI SGKGESTTGF GVYVAVAPAS
VPEFYGNSDK FYGYDPSKDT TESPSTIWVY TPSQKAIGSR
FAQGRPMNND GSFTITMKAP PFEQGKDFVV LTTKAHGVGK
TDHSDDTRTP VTYREATPAP TGPKTPIAPS KQPSKQAAPS
KQVKPSKQAG PNKQSTTPQQ KTAEHRSQTP AAHRTMTKQV
STIGASKVTS GSLTWGIRTS FTSYLRGPIA NGSWKLSGGA
NWNGSAFTFP LTSGSFDPAT KSGSLKYSGS VHMTGHHGIL
DMTLAEPSLQ IKGSTGHLYL DVKSSSMDGK KTNYGRVDFA
TFGVSVSGNA AIKGSPVKLT ATGAKAFAGF YRAGEPMNPL
STNLTLSAEK VSHNVTVDAV TGKVIGDDSG KGAGRGLPVT
P032
(SEQ ID NO: 21)
MAKVRVYELA KELGLSSKQL LGKLNDMGEF VRSASSTIEA
PVVRRLRDQI GSAEPADDAK AAKPAARKSQ TSSKKTSKET
TTAKPAPGPK PGPGPKPTPG PRPGSSSGPK PGRSSAARTS
ATPRPGLIKS SGASSQQEPP AAKPESKPTP KPGQNVPKPH
APAPKPKPGA PSKSPKPGAR PGPRPGGSAG LPSAARPGPR
PGAGRRTGAP RPGNNPFASS QGMGQSRHRS EGGQRSGGSR
SGQGERMPRP GGSQGSRGGS GMPRPNPAMM PKHQSSQIGQ
ATTGRGGRGG GRGRGGSRGS GFGGGFGGGP RGPIGGGRGG
RGGRGTQGAF GRGGGGRRGR KSRKQRRQEF DEMQAPLVGG
VRVRKGNGET VRLRRGASLT DLAEKINAEP AQLVQVLFNL
GEMVTATQSV SDDTLEILGG ELNYQIQVVS PEDEDRELLE
SFDLEFGEDE GDDSDLVARP PVVTVMGHVD HGKTKLLDAL
RHTDVVKGEA GGITQAIGAY QVQTEVDDAE RAITFIDTPG
HEAFTAMRAR GAQSTDIAVL VVAADDGVMP QTVEALNHAK
AADVPIVVAV NKIDKPEADP DKVRGQLTEY GLVPEEYGGD
TMFVNVSART HEGLDDLLEA IVLTADAALD LRANPDMAAQ
GVAIEAHLDK GRGPVATALI QRGTLHIGDS IVAGSSYGRV
RAMINDQGES VDEAAPATPV QVLGLTSVPG AGDNFLVVDD
DRKARQIAEK REARMRAAQQ ARSSRRKTLD QLFEQLEKGE
TEELLLILKG DGAGSVEALE DALAKIDVGD EVDLRVIDRG
VGAITETNVS LAAASNAVIV GENVRPTAHA QRMADEENVD
IRYYSVIYDA IDEIEAALRG MLKPIYEEKA MGTAEIRQIF
RSSKVGTIAG CMITDGTIRR HAKARLVRDG VVVQETEINT
LQREKDAVTE VREGYECGLT LTNYSDIHVG DEVQCYEMVE
KPRDHHHHHH
P035
(SEQ ID NO: 22)
MTQTPVKIAV TGAAGQICYS LLFRIASGSL LGDTPIELRL
LEITPALKAL EGVVMELDDC AFGNLVNIEI GDDPKKVEDG
VNAAFLVGAM PRKAGMERSD LLTKNGAIFA AQGKALNDVA
ADDVRVLVTG NPANTNALIA ATNAVDIPNN HFAALTRLDH
NRAKTQLARK TGKTVNDVRH MTIWGNHSST QYPDVFHAEV
AGQKATNLVN EAWIENEFIP TVAKRGAAII DARGASSAAS
AANATVECMR DWMGSTPEGD WVSMAIPSDG SYGVPEGLIS
SFPVTITNGK VEIVQGLDID DFSRAKIDAS AKELADERDA
VKELGLIHHH HHH
P042
(SEQ ID NO: 23)
MHHHHHHDAV NEIHAKAGDQ ASVSLTAYAD SPFYKKLPSY
VSDSLRDGLF YYTGYYLEDG AAAGLANLTA LHPTDTQDNS
HTLDGDPVLA YCIEKNSLQE RYHQDTGTLT KWSELKPHYN
DNLTNALPKI GWIAKNGYPS KALDELEKQA NAANGNDDAT
GSDHRDAVTA TQAAIWHFSD DFTLKSDDIT EYENGRLISV
PDSAKKHISS LYNYLIKKAV PVSDPLDSTS DARGTSDFAG
AALSFEAGQR GHQDRIIVPI QPISPAPTPT PTPTPTPTPT
PTPTPTPTPT PTPTPTPTPT VDVKVTTTAD QTAEVGKPFG
DTAHITGTVP EGAHITFKLS HKGLLPNMCG DSVITTKPVA
VPAGNHIQPI DIASPKVTVG QAGTYYWVET LTDSNGKVLA
QGKCGEHAET TTVTAPVVPT PTPTMIPVGP SHPGLMPHTG
V
P046
(SEQ ID NO: 24)
MHHHHHHTNT STNAGTTLKR TVALAAAASL AVMGTIAEEA
HAAPAGNLGA HAEKAPTQHT KKAPAEQAKR ATTYTARFAL
TRVHLNVRSG HSTEYRRYGL LRPGDKLLII GEDVRGWTPV
NYRGKTAWVA TRYITKMNRP VGIYAQRGDH NARSKAVQRD
LSTLNYFPAK WVGLPYGPIT TNAVKVFQRA NALKQTGVTD
AVTLGLLKSK ADYQRAAAAE KSSTYKHAEH HSEHSSSTSR
STARPARNTS PDVSTSGIAG SCQASFYDDS QTASGESFSS
SALTAASKTY SFGTRLKVTN KANGQSVVVR VNDRGPYVSG
RCLDLSSAAF SRISSLSAGV ADVTYAKVS
P068
(SEQ ID NO: 25)
MHHHHHHATP IDESQLPVGP QVSVTDSAQH TGPFAASSPL
TITVKPGAPC VRADGYQESM VTRVLDDKGH QVWTGTFDES
KLIGGTGLGT ATFHVGSPAA AFNFHGSERT TYRTLSYCAY
PHYVNGTRER LSQVSVKTFM VDPALN
P069
(SEQ ID NO: 26)
MHHHHHHKNT GRDSAADGKT TVKIGTTEAA NDYWNVIKKK
AAAEGINIDV VSFNDYTQPN VALSQGQIDL NAFQHLLFLA
DYDVKNNDNI TALAATYVVP LNIYSKKYQR LSQLPAGATI
AIPNDATNQG RALLVLQEAG LLKLRGNGSA LSTPADVIAN
QSKVKVKAID AAQTAASLEG VDGAVVNNNF ATDAGLDRNK
VLYHDDATGK SADPYLNIIA VKDADKDNPT YQKVAQLWSD
PDVTKAIIKQ SGGTAKIVSG HSKADLDKIL SGLTTDIKKS
NS
P070
(SEQ ID NO: 27)
MHHHHHHSAS VTAATTMTSR TASAVRTLDL TKTYGSGTTL
VRALRSVTLD IPSGRFTAIM GASGSGKSTL LHCITGLDSS
TSGRVWIGDT EITHLDDDAL TKLRRQHVGF VFQSFNLMPT
LSAADNITLP LRLSHTKVDK EWFDHITGML GITERLGHKP
SQMSGGQQQR VAVARALVTR PDVIVADEPT GALDSEASGD
LLTFLRHCVD DLGQTVVMVT HDHDAAARAD DIVTVTDGQV
STARSQEALA
P071
(SEQ ID NO: 28)
MHHHHHHDQP DADQSQRQVA LGAMQWTINS HFLNALKMMM
HGSWSTSSGA SWNGKAFSFP VSKGSLSSTA QKADVKYSGS
VHFTGVGGRM DLTLADPEIV VNNGDDHVIM SVKSKTMVGQ
SVDYGRITFV DMTGSITQGQ NDAAQVVGTN VKLNSQAVDA
FAGFYQAGKP MDDIDSSLKL GPQSSRPEPT VTPKPSVTPK
PSVKPKPTVA PKPSVKPKPR VSPKPSAKPK PTVAPKPDQS
TIMAPQSDAA TEAAAGEAPS AGAHKIIRKQ VCTVVPGASR
ATSPTSTPAK VGKGSMNWGV STSFVNALKG PMQGKWTMSG
GASADGTREN FPFSGGNYDR ATKTGNLNFS GGVHFTGVHG
LLDLSLSDPS LSLKNGKGVM KLTLNAGGMA PFKGRVDFAN
VSVVPSGSGF TATSVVLTGN GVQVWNGFYK KGHSMDNFSV
SLGDKSDSAS AAKQNSASLV KKSAASPTKR ECHDVLVDAK
TGQIISGSNG SLPNTGV
P022F1
(SEQ ID NO: 29)
MHHHHHHLSL LRRLLRWSDQ SQQSPPPPPP QAEASSNRPR
SVAQAAIATD GKGIIDKDCR DAVINDAKLR AAIAGALVKA
GESSADAVAL APRIAKEMAK EGVLLINHHK LKALIGAQLG
LLTDAKIQRA AAAVDLGIKA TLAATIIP
P022F2
(SEQ ID NO: 30)
MHHHHHHGVL LINHHKLKAL IGAQLGLLTD AKIQRAAAAV
DLGIKATLAA TIIPNALHSA AFKDAVVANL VAAGVDKKLA
KATAVAIAAT ALNPALGPIA KTEAIKAEIA AQAALLVGRG
VHLKKAAIEH IIGRSFDAAV ATAIVSSP
P022F3
(SEQ ID NO: 31)
MHHHHHHGVH LKKAAIEHII GRSFDAAVAT AIVSSPILNA
RIVTHLVRAG IDKSLAVQIA PRIIDRLAKE PLLALNTAKL
MKNITRQIVD VITADKAIKT AEQLEKELPA LDDLVKKACS
CPKPTPTPTP TPTPTPKPTP TPTPKPTPTP KPKPTPAPAP
TSG
P022F4
(SEQ ID NO: 32)
MHHHHHHPLL ALNTAKLMKN ITRQIVDVIT ADKAIKTAEQ
LEKELPALDD LVKKACSCPK PTPTPTPTPT PTPKPTPTPT
PKPTPTPKPK PTPAPAPTSG ATSDESTSRS GGHSQGGSGT
HYIHHGVAPV LTHSSDLPST GF
P022F5
(SEQ ID NO: 33)
MHHHHHHLSL LRRLLRWSDQ SQQSPPPPPP QAEASSNRPR
SVAQAAIATD GKGIIDKDCR DAVINDAKLR AAIAGALVKA
GESSADAVAL APRIAKEMAK EGVLLINHHK LKALIGAQLG
LLTDAKIQRA AAAVDLGIKA TLAATIIPNA LHSAAFKDAV
VANLVAAGVD KKLAKATAVA IAATALNPAL GPIAKTEAIK
AEIAAQAALL VGRGVHLKKA AIEHIIGRSF DAAVATAIVS
SP P022F6
(SEQ ID NO: 34)
MHHHHHHLSL LRRLLRWSDQ SQQSPPPPPP QAEASSNRPR
SVAQAAIATD GKGIIDKDCR DAVINDAKLR AAIAGALVKA
GESSADAVAL APRIAKEMAK EGVLLINHHK LKALIGAQLG
LLTDAKIQRA AAAVDLGIKA TLAATIIPNA LHSAAFKDAV
VANLVAAGVD KKLAKATAVA IAATALNPAL GPIAKTEAIK
AEIAAQAALL VGRGVHLKKA AIEHIIGRSF DAAVATAIVS
SPILNARIVT HLVRAGIDKS LAVQIAPRII DRLAKEPLLA
LNTAKLMKNI TRQIVDVITA DKAIKTAEQL EKELPALDDL
VKKACSCPKP TPTPTPTPTP TPKPTPTPTP KPTPTPKPKP
TPAPAPTSG
P022F7
(SEQ ID NO: 35)
MHHHHHHGVL LINHHKLKAL IGAQLGLLTD AKIQRAAAAV
DLGIKATLAA TIIPNALHSA AFKDAVVANL VAAGVDKKLA
KATAVAIAAT ALNPALGPIA KTEAIKAEIA AQAALLVGRG
VHLKKAAIEH IIGRSFDAAV ATAIVSSPIL NARIVTHLVR
AGIDKSLAVQ IAPRIIDRLA KEPLLALNTA KLMKNITRQI
VDVITADKAI KTAEQLEKEL PALDDLVKKA CSCPKPTPTP
TPTPTPTPKP TPTPTPKPTP TPKPKPTPAP APTSGATSDE
STSRSGGHSQ GGSGTHYIHH GVAPVLTHSS DLPSTGF
P022F8
(SEQ ID NO: 36)
MHHHHHHPPQ AEASSNRPRS VAQAAIATDG KGIIDKDCRD
AVINDAKLRA AIAGALVKAG FSSADAVALA PRIAKEMAKE
GVLLINHHKL KALIGAQLGL LTDAKIQRAA AAVDLGIKAT
LAATIIPNAL HSAAFKDAVV ANLVAAGVDK KLAKATAVAI
AATALNPALG PIAKTEAIKA EIAAQAALLV GRGVHLKKAA
IEHIIGRSFD AAVATAIVSS PILNARIVTH LVRAGIDKSL
AVQIAPRIID RLAKEPLLAL NTAKLMKNIT RQIVDVITAD
KAIKTAEQLE KELPALDDLV KKACS
P022F9
(SEQ ID NO: 37)
MHHHHHHSSN RPRSVAQAAI ATDGKGIIDK DCRDAVINDA
KLRAAIAGAL VKAGFSSADA VALAPRIAKE MAKEGVLLIN
HHKLKALIGA QLGLLTDAKI QRAAAAVDLG IKATLAATII
PNALHSAAFK DAVVANLVAA GVDKKLAKAT AVAIAATALN
PALGPIAKTE AIKAEIAAQA ALLVGRGVHL KKAAIEHIIG
RSFDAAVATA IVSSPILNAR IVTHLVRAGI DKSLAVQIAP
RIIDRLAKEP LLALNTAKLM KNITRQIVDV ITADKAIKTA
EQLEKELPAL DDLVKKACSC PKPTPTPTPT PT
P022F10
(SEQ ID NO: 38)
MHHHHHHSSN RPRSVAQAAI ATDGKGIIDK DCRDAVINDA
KLRAAIAGAL VKAGFSSADA VALAPRIAKE MAKEGVLLIN
HHKLKALIGA QLGLLTDAKI QRAAAAVDLG IKATLAATII
PNAL
P022F11
(SEQ ID NO: 39)
MHHHHHHSAA FKDAVVANLV AAGVDKKLAK ATAVAIAATA
LNPALGPIAK TEAIKAEIAA QAALLVGRGV HLKKAAIEHI
IGRSFDAAVA TAIVSSPILN ARIVTHLVRA GIDKSLAVQI
APRIIDRLAK EPLLALNT
P022F12
(SEQ ID NO: 40)
MHHHHHHKAE IAAQAALLVG RGVHLKKAAI EHIIGRSFDA
AVATAIVSSP ILNARIVTHL VRAGIDKSLA VQIAPRIIDR
LAKEPLLALN TAKLMKNITR QIVDVITADK AIKTAEQLEK
ELPALDDLVK KACSCPKPTP TPTPTPTPTP KPTPTPTPKP
TPTPKPKPTP APAPTSG
P022F13
(SEQ ID NO: 41)
MHHHHHHKAE IAAQAALLVG RGVHLKKAAI EHIIGRSFDA
AVATAIVSSP ILNARIVTHL VRAGIDKSLA VQIAPRIIDR
LAKEPLLALN TAKLMKNITR QIVDVITADK AIKTAEQLEK
ELPALDDLVK KACSCPKPTP TPTPTPTPTP KPTPTPTPKP
TPTPKPKPTP APAPTSGATS DESTSRSGGH SQGGSGTHYI
HHGVAPVLTH SSDLPSTGF
P022F14
(SEQ ID NO: 42)
MHHHHHHSSN RPRSVAQAAI ATDGKGIIDK DCRDAVINDA
KLRAAIAGAL VKAGFSSADA VALAPRIAKE MAKEGVLLIN
HHKLKALIGA QLGLLTDAKI QRAAAAVDLG IKATLAATII
PNALHSAAFK DAVVANLVAA GVDKKLAKAT AVAIAATALN
PALGPIAKTE AIKAEIAAQA ALLVGRGVHL KKAAIEHIIG
RSFDAAVATA IVSSPILNAR IVTHLVRAGI DKSLAVQIAP
RIIDRLAKEP LLALNTAKLM KNITRQIVDV ITADKAIKTA
EQLEKELPAL DDLVKKACS
P027F1
(SEQ ID NO: 43)
MHHHHHHGSA AFKNAVIANL VAAGIDKHLA RATAVAIVAT
ALNPALGPIA KFELIKAEIA AQAALLIRRG VHLQKAAIEH
VIGRAFDAAV ATAIISSPIL SARIVTHL VR AGIDKSIAIS
LAPHIVKRLA KEPLLAFNT
H2
(SEQ ID NO: 44)
MHHHHHHSAA FKDAVVANLV AAGVDKKLAK ATAVAIAATA
LNPALGPIAK TEAIKAEIAA QAALLVGRGV HLKKAAIEHI
IGRSFDAAVA TAIVSSPILN ARIVTHLVRA GIDKSLAVQI
APRIIDRLAK EPLLALNTNA LGSAAFKNAV IANLVAAGID
KHLARATAVA IVATALNPAL GPIAKFELIK AEIAAQAALL
IRRGVHLQKA AIEHVIGRAF DAAVATAIIS SPILSARIVT
HLVRAGIDKS IAISLAPHIV KRLAKEPLLA FNT
H3
(SEQ ID NO: 45)
MHHHHHHSSN RPRSVAQAAI ATDGKGIIDK DCRDAVINDA
KLRAAIAGAL VKAGFSSADA VALAPRIAKE MAKEGVLLIN
HHKLKALIGA QLGLLTDAKI QRAAAAVDLG IKATLAATII
PNALASNGNS SITQSAAFSP RATTKISEDC RKAIINDLKL
RGAIVGALVK AGLSAADAAA LAPRIAAEMA AEGTLTINHH
RLKVLVASQL GLVADAAVQH AAAAIDLSFK AILGASIIPN
AL
H4
(SEQ ID NO: 46)
MHHHHHHSSN RPRSVAQAAI ATDGKGIIDK DCRDAVINDA
KLRAAIAGAL VKAGFSSADA VALAPRIAKE MAKEGVLLIN
HHKLKALIGA QLGLLTDAKI QRAAAAVDLG IKATLAATII
PNALGSAAFK NAVIANLVAA GIDKHLARAT AVAIVATALN
PALGPIAKFE LIKAEIAAQA ALLIRRGVHL QKAAIEHVIG
RAFDAAVATA IISSPILSAR IVTHLVRAGI DKSIAISLAP
HIVKRLAKEP LLAFNTAKLM KNITRQIVDV ITADKAIKTA
EQLEKELPAL DDLVKKACSC PKPTPTPTPT PT
H4-V1
(SEQ ID NO: 47)
MSSNRPRSVA QAAIATDGKG IIDKDCRDAV INDAKLRAAI
AGALVKAGFS SADAVALAPR IAKEMAKEGV LLINHHKLKA
LIGAQLGLLT DAKIQRAAAA VDLGIKATLA ATIIPNALGS
AAFKNAVIAN LVAAGIDKHL ARATAVAIVA TALNPALGPI
AKFELIKAEI AAQAALLIRR GVHLQKAAIE HVIGRAFDAA
VATAIISSPI LSARIVTHLV RAGIDKSIAI SLAPHIVKRL
AKEPLLAFNT AKLMKNITRQ IVDVITADKA IKTAEQLEKE
LPALDDLVKK ACSCPKPTPT PTPTPT
H4-V2
(SEQ ID NO: 48)
MSSNRPRSVA QAAIATDGKG IIDKDSRDAV INDAKLRAAI
AGALVKAGFS SADAVALAPR IAKEMAKEGV LLINHHKLKA
LIGAQLGLLT DAKIQRAAAA VDLGIKATLA ATIIPNALGS
AAFKNAVIAN LVAAGIDKHL ARATAVAIVA TALNPALGPI
AKFELIKAEI AAQAALLIRR GVHLQKAAIE HVIGRAFDAA
VATAIISSPI LSARIVTHLV RAGIDKSIAI SLAPHIVKRL
AKEPLLAFNT AKLMKNITRQ IVDVITADKA IKTAEQLEKE
LPALDDLVKK ACSCPKPTPT PTPTPT
H4-V3
(SEQ ID NO: 49)
MSSNRPRSVA QAAIATDGKG IIDKDSRDAV INDAKLRAAI
AGALVKAGFS SADAVALAPR IAKEMAKEGV LLINHHKLKA
LIGAQLGLLT DAKIQRAAAA VDLGIKATLA ATIIPNALGS
AAFKNAVIAN LVAAGIDKHL ARATAVAIVA TALNPALGPI
AKFELIKAEI AAQAALLIRR GVHLQKAAIE HVIGRAFDAA
VATAIISSPI LSARIVTHLV RAGIDKSIAI SLAPHIVKRL
AKEPLLAFNT AKLMKNITRQ IVDVITADKA IKTAEQLEKE
LPALDDLVKK ASSPPKPTPT PTPTPT
H5
(SEQ ID NO: 50)
MHHHHHHGSA AFKNAVIANL VAAGIDKHLA RATAVAIVAT
ALNPALGPIA KFELIKAEIA AQAALLIRRG VHLQKAAIEH
VIGRAFDAAV ATAIISSPIL SARIVTHLVR AGIDKSIAIS
LAPHIVKRLA KEPLLAFNTA KLMKNITRQI VDVITADKAI
KTAEQLEKEL PALDDLVKKA CSCPKPTPTP TPTPT
PRO-H4-V3 human codon bias
(SEQ ID NO: 67)
ATGAGCAGCAACAGACCCAGAAGCGTGGCCCAGGCCGCCA
TCGCCACCGACGGCAAGGGCATCATCGACAAGGACAGCAG
AGACGCCGTGATCAACGACGCCAAGCTGAGAGCCGCCATC
GCCGGCGCCCTGGTGAAGGCCGGCTTCAGCAGCGCCGACG
CCGTGGCCCTGGCCCCCAGAATCGCCAAGGAGATGGCCAA
GGAGGGCGTGCTGCTGATCAACCACCACAAGCTGAAGGCC
CTGATCGGCGCCCAGCTGGGCCTGCTGACCGACGCCAAGA
TCCAGAGAGCCGCCGCCGCCGTGGACCTGGGCATCAAGGC
CACCCTGGCCGCCACCATCATCCCCAACGCCCTGGGCAGC
GCCGCCTTCAAGAACGCCGTGATCGCCAACCTGGTGGCCG
CCGGCATCGACAAGCACCTGGCCAGAGCCACCGCCGTGGC
CATCGTGGCCACCGCCCTGAACCCCGCCCTGGGCCCCATC
GCCAAGTTCGAGCTGATCAAGGCCGAGATCGCCGCCCAGG
CCGCCCTGCTGATCAGAAGAGGCGTGCACCTGCAGAAGGC
CGCCATCGAGCACGTGATCGGCAGAGCCTTCGACGCCGCC
GTGGCCACCGCCATCATCAGCAGCCCCATCCTGAGCGCCA
GAATCGTGACCCACCTGGTGAGAGCCGGCATCGACAAGAG
CATCGCCATCAGCCTGGCCCCCCACATCGTGAAGAGACTG
GCCAAGGAGCCCCTGCTGGCCTTCAACACCGCCAAGCTGA
TGAAGAACATCACCAGACAGATCGTGGACGTGATCACCGC
CGACAAGGCCATCAAGACCGCCGAGCAGCTGGAGAAGGAG
CTGCCCGCCCTGGACGACCTGGTGAAGAAGGCCAGCAGCC
CCCCCAAGCCCACCCCCACCCCCACCCCCACCCCCACC
PRO-H4-V3 high-GC + human codon bias
(SEQ ID NO: 68)
ATGAGCAGCAACCGGCCCCGGAGCGTGGCCCAGGCCGCCA
TCGCCACCGACGGCAAGGGCATCATCGACAAGGACAGCCG
GGACGCCGTGATCAACGACGCCAAGCTGCGGGCCGCCATC
GCCGGCGCCCTGGTGAAGGCCGGCTTCAGCAGCGCCGACG
CCGTGGCCCTGGCCCCCCGGATCGCCAAGGAGATGGCCAA
GGAGGGCGTGCTGCTGATCAACCACCACAAGCTGAAGGCC
CTGATCGGCGCCCAGCTGGGCCTGCTGACCGACGCCAAGA
TCCAGCGGGCCGCCGCCGCCGTGGACCTGGGCATCAAGGC
CACCCTGGCCGCCACCATCATCCCCAACGCCCTGGGCAGC
GCCGCCTTCAAGAACGCCGTGATCGCCAACCTGGTGGCCG
CCGGCATCGACAAGCACCTGGCCCGGGCCACCGCCGTGGC
CATCGTGGCCACCGCCCTGAACCCCGCCCTGGGCCCCATC
GCCAAGTTCGAGCTGATCAAGGCCGAGATCGCCGCCCAGG
CCGCCCTGCTGATCCGGCGGGGCGTGCACCTGCAGAAGGC
CGCCATCGAGCACGTGATCGGCCGGGCCTTCGACGCCGCC
GTGGCCACCGCCATCATCAGCAGCCCCATCCTGAGCGCCC
GGATCGTGACCCACCTGGTGCGGGCCGGCATCGACAAGAG
CATCGCCATCAGCCTGGCCCCCCACATCGTGAAGCGGCTG
GCCAAGGAGCCCCTGCTGGCCTTCAACACCGCCAAGCTGA
TGAAGAACATCACCCGGCAGATCGTGGACGTGATCACCGC
CGACAAGGCCATCAAGACCGCCGAGCAGCTGGAGAAGGAG
CTGCCCGCCCTGGACGACCTGGTGAAGAAGGCCAGCAGCC
CCCCCAAGCCCACCCCCACCCCCACCCCCACCCCCACC
PRO-028-V7 human codon bias
(SEQ ID NO: 69)
ATGGCCGGCCCCACCGTGACCGTGATCCCCGTGGGCAGAG
AGGGCGGCGACATCACCATCAGCGGCAAGGGCTTCAGCAC
CACCGGCTTCGGCGTGTACGTGGCCGTGGCCCCCGCCAGC
GTGCCCGAGTTCTACGGCAACAGCGACAAGTTCTACGGCT
ACGACCCCAGCAAGGACACCACCGAGAGCCCCAGCACCAT
CTGGGTGTACACCCCCAGCCAGAAGGCCATCGGCAGCAGA
TTCGCCCAGGGCAGACCCATGAACAACGACGGCAGCTTCA
CCATCACCATGAAGGCCCCCCCCTTCGAGCAGGGCAAGGA
CTTCGTGGTGCTGACCACCAAGGCCCACGGCGTGGGCAAG
ACCGACCACAGCGACGACACCAGAACCCCCGTGACCTACA
GAGAGGCCACCCCCGCCCCCACCGGCCCCAAGACCCCCAT
CGCCCCCAGCAAGCAGCCCAGCAAGCAGGCCGCCCCCAGC
AAGCAGGTGAAGCCCAGCAAGCAGGCCGGCCCCAACAAGC
AGAGCACCACCCCCCAGCAGAAGACCGCCGAGCACAGAAG
CCAGACCCCCGCCGCCCACAGAACCATGACCAAGCAGGTG
AGCACCATCGGCGCCAGCAAGGTGACCAGCGGCAGCCTGA
CCTGGGGCATCAGAACCAGCTTCACCAGCTACCTGAGAGG
CCCCATCGCCAACGGCAGCTGGAAGCTGAGCGGCGGCGCC
AACTGGAACGGCAGCGCCTTCACCTTCCCCCTGACCAGCG
GCAGCTTCGACCCCGCCACCAAGAGCGGCAGCCTGAAGTA
CAGCGGCAGCGTGCACATGACCGGCCACCACGGCATCCTG
GACATGACCCTGGCCGAGCCCAGCCTGCAGATCAAGGGCA
GCACCGGCCACCTGTACCTGGACGTGAAGAGCAGCAGCAT
GGACGGCAAGAAGACCAACTACGGCAGAGTGGACTTCGCC
ACCTTCGGCGTGAGCGTGAGCGGCAACGCCGCCATCAAGG
GCAGCCCCGTGAAGCTGACCGCCACCGGCGCCAAGGCCTT
CGCCGGCTTCTACAGAGCCGGCGAGCCCATGAACCCCCTG
AGCACCAACCTGACCCTGAGCGCCGAGAAGGTGAGCCACA
ACGTGACCGTGGACGCCGTGACCGGCAAGGTGATCGGCGA
CGACAGCGGCAAGGGCGCCGGCAGAGGCCTGCCCGTGACC
PRO-028-V7 high-GC + human codon bias
(SEQ ID NO: 70)
ATGGCCGGCCCCACCGTGACCGTGATCCCCGTGGGCCGGG
AGGGCGGCGACATCACCATCAGCGGCAAGGGCTTCAGCAC
CACCGGCTTCGGCGTGTACGTGGCCGTGGCCCCCGCCAGC
GTGCCCGAGTTCTACGGCAACAGCGACAAGTTCTACGGCT
ACGACCCCAGCAAGGACACCACCGAGAGCCCCAGCACCAT
CTGGGTGTACACCCCCAGCCAGAAGGCCATCGGCAGCCGG
TTCGCCCAGGGCCGGCCCATGAACAACGACGGCAGCTTCA
CCATCACCATGAAGGCCCCCCCCTTCGAGCAGGGCAAGGA
CTTCGTGGTGCTGACCACCAAGGCCCACGGCGTGGGCAAG
ACCGACCACAGCGACGACACCCGGACCCCCGTGACCTACC
GGGAGGCCACCCCCGCCCCCACCGGCCCCAAGACCCCCAT
CGCCCCCAGCAAGCAGCCCAGCAAGCAGGCCGCCCCCAGC
AAGCAGGTGAAGCCCAGCAAGCAGGCCGGCCCCAACAAGC
AGAGCACCACCCCCCAGCAGAAGACCGCCGAGCACCGGAG
CCAGACCCCCGCCGCCCACCGGACCATGACCAAGCAGGTG
AGCACCATCGGCGCCAGCAAGGTGACCAGCGGCAGCCTGA
CCTGGGGCATCCGGACCAGCTTCACCAGCTACCTGCGGGG
CCCCATCGCCAACGGCAGCTGGAAGCTGAGCGGCGGCGCC
AACTGGAACGGCAGCGCCTTCACCTTCCCCCTGACCAGCG
GCAGCTTCGACCCCGCCACCAAGAGCGGCAGCCTGAAGTA
CAGCGGCAGCGTGCACATGACCGGCCACCACGGCATCCTG
GACATGACCCTGGCCGAGCCCAGCCTGCAGATCAAGGGCA
GCACCGGCCACCTGTACCTGGACGTGAAGAGCAGCAGCAT
GGACGGCAAGAAGACCAACTACGGCCGGGTGGACTTCGCC
ACCTTCGGCGTGAGCGTGAGCGGCAACGCCGCCATCAAGG
GCAGCCCCGTGAAGCTGACCGCCACCGGCGCCAAGGCCTT
CGCCGGCTTCTACCGGGCCGGCGAGCCCATGAACCCCCTG
AGCACCAACCTGACCCTGAGCGCCGAGAAGGTGAGCCACA
ACGTGACCGTGGACGCCGTGACCGGCAAGGTGATCGGCGA
CGACAGCGGCAAGGGCGCCGGCCGGGGCCTGCCCGTGACC
PRO-H4-V3 ORI000137 rank_frequency
(SEQ ID NO: 71)
ATGAGCAGCAACCGGCCCCGGAGCGTGGCCCAGGCCGCCA
TCGCCACCGACGGCAAGGGCATCATCGACAAGGACAGCCG
GGACGCCGTGATCAACGACGCCAAGCTGCGGGCCGCCATC
GCCGGCGCCCTGGTGAAGGCCGGCTTCAGCAGCGCCGACG
CCGTGGCCCTGGCCCCCCGGATCGCCAAGGAGATGGCCAA
GGAGGGCGTGCTGCTGATCAACCACCACAAGCTGAAGGCC
CTGATCGGCGCCCAGCTGGGCCTGCTGACCGACGCCAAGA
TCCAGCGGGCCGCCGCCGCCGTGGACCTGGGCATCAAGGC
CACCCTGGCCGCCACCATCATCCCCAACGCCCTGGGCAGC
GCCGCCTTCAAGAACGCCGTGATCGCCAACCTGGTGGCCG
CCGGCATCGACAAGCACCTGGCCCGGGCCACCGCCGTGGC
CATCGTGGCCACCGCCCTGAACCCCGCCCTGGGCCCCATC
GCCAAGTTCGAGCTGATCAAGGCCGAGATCGCCGCCCAGG
CCGCCCTGCTGATCCGGCGGGGCGTGCACCTGCAGAAGGC
CGCCATCGAGCACGTGATCGGCCGGGCCTTCGACGCCGCC
GTGGCCACCGCCATCATCAGCAGCCCCATCCTGAGCGCCC
GGATCGTGACCCACCTGGTGCGGGCCGGCATCGACAAGAG
CATCGCCATCAGCCTGGCCCCCCACATCGTGAAGCGGCTG
GCCAAGGAGCCCCTGCTGGCCTTCAACACCGCCAAGCTGA
TGAAGAACATCACCCGGCAGATCGTGGACGTGATCACCGC
CGACAAGGCCATCAAGACCGCCGAGCAGCTGGAGAAGGAG
CTGCCCGCCCTGGACGACCTGGTGAAGAAGGCCAGCAGCC
CCCCCAAGCCCACCCCCACCCCCACCCCCACCCCCACC
PRO-028-V7 ORI000135 rank_frequency
(SEQ ID NO: 72)
ATGGCCGGCCCCACCGTGACCGTGATCCCCGTGGGCCGGG
AGGGCGGCGACATCACCATCAGCGGCAAGGGCTTCAGCAC
CACCGGCTTCGGCGTGTACGTGGCCGTGGCCCCCGCCAGC
GTGCCCGAGTTCTACGGCAACAGCGACAAGTTCTACGGCT
ACGACCCCAGCAAGGACACCACCGAGAGCCCCAGCACCAT
CTGGGTGTACACCCCCAGCCAGAAGGCCATCGGCAGCCGG
TTCGCCCAGGGCCGGCCCATGAACAACGACGGCAGCTTCA
CCATCACCATGAAGGCCCCCCCCTTCGAGCAGGGCAAGGA
CTTCGTGGTGCTGACCACCAAGGCCCACGGCGTGGGCAAG
ACCGACCACAGCGACGACACCCGGACCCCCGTGACCTACC
GGGAGGCCACCCCCGCCCCCACCGGCCCCAAGACCCCCAT
CGCCCCCAGCAAGCAGCCCAGCAAGCAGGCCGCCCCCAGC
AAGCAGGTGAAGCCCAGCAAGCAGGCCGGCCCCAACAAGC
AGAGCACCACCCCCCAGCAGAAGACCGCCGAGCACCGGAG
CCAGACCCCCGCCGCCCACCGGACCATGACCAAGCAGGTG
AGCACCATCGGCGCCAGCAAGGTGACCAGCGGCAGCCTGA
CCTGGGGCATCCGGACCAGCTTCACCAGCTACCTGCGGGG
CCCCATCGCCAACGGCAGCTGGAAGCTGAGCGGCGGCGCC
AACTGGAACGGCAGCGCCTTCACCTTCCCCCTGACCAGCG
GCAGCTTCGACCCCGCCACCAAGAGCGGCAGCCTGAAGTA
CAGCGGCAGCGTGCACATGACCGGCCACCACGGCATCCTG
GACATGACCCTGGCCGAGCCCAGCCTGCAGATCAAGGGCA
GCACCGGCCACCTGTACCTGGACGTGAAGAGCAGCAGCAT
GGACGGCAAGAAGACCAACTACGGCCGGGTGGACTTCGCC
ACCTTCGGCGTGAGCGTGAGCGGCAACGCCGCCATCAAGG
GCAGCCCCGTGAAGCTGACCGCCACCGGCGCCAAGGCCTT
CGCCGGCTTCTACCGGGCCGGCGAGCCCATGAACCCCCTG
AGCACCAACCTGACCCTGAGCGCCGAGAAGGTGAGCCACA
ACGTGACCGTGGACGCCGTGACCGGCAAGGTGATCGGCGA
CGACAGCGGCAAGGGCGCCGGCCGGGGCCTGCCCGTGACC
PRO-H4-V3 ORI000137 bicodon_optimized
(SEQ ID NO: 73)
ATGAGCAGCAACCGGCCCCGCTCTGTGGCCCAGGCCGCCA
TCGCCACAGATGGCAAGGGCATCATCGACAAGGACAGCAG
AGATGCTGTGATCAATGATGCCAAGCTGCGGGCTGCCATC
GCCGGGGCCCTGGTGAAGGCCGGCTTCAGCTCTGCTGATG
CTGTGGCCCTGGCCCCCCGCATCGCCAAGGAGATGGCCAA
GGAGGGCGTGCTGCTCATCAACCACCACAAGCTGAAGGCC
CTGATTGGGGCCCAGCTGGGCCTGCTGACAGATGCCAAGA
TCCAGCGGGCTGCTGCTGCTGTGGACCTGGGCATCAAGGC
CACCCTGGCCGCCACCATCATCCCCAATGCCCTGGGCTCT
GCTGCCTTCAAGAATGCCGTCATCGCCAACCTGGTGGCCG
CCGGCATCGACAAGCACCTGGCCCGGGCCACAGCTGTGGC
CATCGTGGCCACCGCCCTGAACCCTGCCCTGGGCCCCATC
GCCAAGTTTGAGCTGATCAAGGCCGAGATTGCTGCCCAGG
CCGCCCTGCTCATCCGCCGGGGCGTGCACCTGCAGAAGGC
TGCCATCGAGCACGTGATTGGCCGGGCCTTTGATGCTGCT
GTGGCCACCGCCATCATCAGCAGCCCCATCCTGTCTGCCC
GCATCGTGACCCACCTGGTGCGGGCCGGCATCGACAAGAG
CATCGCCATCAGCCTGGCCCCCCACATCGTGAAGCGGCTG
GCCAAGGAGCCCCTGCTGGCCTTCAACACAGCCAAGCTGA
TGAAGAACATCACCCGGCAGATCGTGGATGTGATCACCGC
TGACAAGGCCATCAAGACAGCTGAGCAGCTGGAGAAGGAG
CTGCCTGCCCTGGATGACCTGGTGAAGAAGGCCAGCAGCC
CCCCCAAGCCCACCCCCACCCCCACCCCCACCCCCACC
PRO-028-V7 ORI000135 bicodon_optimized
(SEQ ID NO: 74)
ATGGCCGGCCCCACTGTGACTGTCATCCCTGTGGGCCGGG
AGGGGGGGGACATCACCATCAGCGGCAAGGGCTTCAGCAC
CACCGGCTTTGGCGTGTATGTGGCTGTGGCCCCAGCCAGC
GTGCCTGAGTTCTACGGCAACAGTGACAAGTTCTACGGCT
ATGACCCCAGCAAGGACACCACCGAGAGCCCCAGCACCAT
CTGGGTGTACACCCCCAGCCAGAAGGCCATCGGCAGCCGC
TTTGCCCAGGGCCGGCCCATGAACAATGATGGCTCCTTCA
CCATCACCATGAAGGCCCCCCCATTTGAGCAGGGCAAGGA
CTTCGTGGTGCTGACCACCAAGGCCCACGGCGTGGGCAAG
ACAGACCACAGTGATGACACCCGCACCCCTGTGACCTACC
GGGAGGCCACCCCAGCCCCCACTGGCCCCAAGACCCCCAT
CGCCCCCAGCAAGCAGCCCAGCAAGCAGGCCGCCCCCAGC
AAGCAGGTGAAGCCCAGCAAGCAGGCCGGCCCCAACAAGC
AGAGCACCACCCCCCAGCAGAAGACAGCTGAGCACCGCAG
CCAGACCCCAGCTGCCCACCGCACCATGACCAAGCAGGTG
TCCACCATCGGGGCCAGCAAGGTGACCAGCGGCAGCCTGA
CCTGGGGCATCCGCACCTCCTTCACCAGCTACCTGCGGGG
CCCCATCGCCAACGGCTCCTGGAAGCTGTCTGGAGGGGCC
AACTGGAATGGCAGCGCCTTCACCTTCCCCCTGACCAGCG
GCAGCTTTGACCCAGCCACCAAGAGCGGCAGCCTGAAGTA
CAGCGGCTCTGTGCACATGACTGGCCACCACGGCATCCTG
GACATGACCCTGGCCGAGCCCAGCCTGCAGATCAAGGGCA
GCACCGGCCACCTGTACCTGGATGTGAAGAGCAGCTCCAT
GGATGGCAAGAAGACCAACTACGGCCGGGTGGACTTTGCC
ACCTTCGGGGTGTCTGTGTCTGGAAATGCCGCCATCAAGG
GCAGCCCTGTGAAGCTGACAGCCACCGGGGCCAAGGCCTT
TGCTGGCTTCTACAGAGCTGGGGAGCCCATGAACCCCCTG
AGCACCAACCTGACCCTGTCTGCTGAGAAGGTGTCCCACA
ACGTGACTGTGGATGCTGTGACTGGCAAGGTGATTGGAGA
TGATTCTGGCAAGGGGGCCGGCCGGGGCCTGCCTGTGACC
PRO-H4-V3 ORI000137 bicodon_optimized
hybrid model
(SEQ ID NO: 75)
ATGAGCTCCAACCGCCCCCGCTCCGTGGCCCAGGCCGCCA
TCGCCACAGATGGCAAGGGCATCATCGACAAGGACAGCAG
AGATGCTGTGATCAATGATGCCAAGCTGCGGGCTGCCATC
GCCGGGGCCCTGGTGAAGGCCGGCTTCAGCTCTGCTGATG
CTGTGGCCCTGGCCCCCCGCATCGCCAAGGAGATGGCCAA
GGAGGGCGTGCTGCTCATCAACCACCACAAGCTGAAGGCC
CTGATTGGGGCCCAGCTGGGCCTGCTGACAGATGCCAAGA
TCCAGCGGGCTGCTGCTGCTGTGGACCTGGGCATCAAGGC
CACCCTGGCCGCCACCATCATCCCCAATGCCCTGGGCTCT
GCTGCCTTCAAGAATGCCGTCATCGCCAACCTGGTGGCCG
CCGGCATCGACAAGCACCTGGCCCGGGCCACAGCTGTGGC
CATCGTGGCCACCGCCCTGAACCCTGCCCTGGGCCCCATC
GCCAAGTTTGAGCTGATCAAGGCCGAGATTGCTGCCCAGG
CCGCCCTGCTCATCCGCCGGGGCGTGCACCTGCAGAAGGC
TGCCATCGAGCACGTGATTGGCCGGGCCTTTGATGCTGCT
GTGGCCACCGCCATCATCAGCAGCCCCATCCTGTCTGCCC
GCATCGTGACCCACCTGGTGCGGGCCGGCATCGACAAGAG
CATCGCCATCAGCCTGGCCCCCCACATCGTGAAGCGGCTG
GCCAAGGAGCCCCTGCTGGCCTTCAACACAGCCAAGCTGA
TGAAGAACATCACCCGGCAGATCGTGGATGTGATCACCGC
TGACAAGGCCATCAAGACAGCTGAGCAGCTGGAGAAGGAG
CTGCCTGCCCTGGATGACCTGGTGAAGAAGGCCAGCAGCC
CCCCCAAGCCCACCCCCACCCCCACCCCCACCCCCACC
PRO-028-V7 ORI000135 bicodon_optimized
hybrid model
(SEQ ID NO: 76)
ATGGCCGGGCCCACCGTGACCGTGATCCCAGTGGGCCGGG
AGGGCGGCGACATCACCATCAGCGGCAAGGGCTTCAGCAC
CACCGGCTTTGGCGTGTATGTGGCTGTGGCCCCAGCCAGC
GTGCCTGAGTTCTACGGCAACAGTGACAAGTTCTACGGCT
ATGACCCCAGCAAGGACACCACCGAGAGCCCCAGCACCAT
CTGGGTGTACACCCCCAGCCAGAAGGCCATCGGCAGCCGC
TTTGCCCAGGGCCGGCCCATGAACAATGATGGCTCCTTCA
CCATCACCATGAAGGCCCCCCCATTTGAGCAGGGCAAGGA
CTTCGTGGTGCTGACCACCAAGGCCCACGGCGTGGGCAAG
ACAGACCACAGTGATGACACCCGCACCCCTGTGACCTACC
GGGAGGCCACCCCAGCCCCCACTGGCCCCAAGACCCCCAT
CGCCCCCAGCAAGCAGCCCAGCAAGCAGGCCGCCCCCAGC
AAGCAGGTGAAGCCCAGCAAGCAGGCCGGCCCCAACAAGC
AGAGCACCACCCCCCAGCAGAAGACAGCTGAGCACCGCAG
CCAGACCCCAGCTGCCCACCGCACCATGACCAAGCAGGTG
TCCACCATCGGGGCCAGCAAGGTGACCAGCGGCAGCCTGA
CCTGGGGCATCCGCACCTCCTTCACCAGCTACCTGCGGGG
CCCCATCGCCAACGGCTCCTGGAAGCTGTCTGGAGGGGCC
AACTGGAATGGCAGCGCCTTCACCTTCCCCCTGACCAGCG
GCAGCTTTGACCCAGCCACCAAGAGCGGCAGCCTGAAGTA
CAGCGGCTCTGTGCACATGACTGGCCACCACGGCATCCTG
GACATGACCCTGGCCGAGCCCAGCCTGCAGATCAAGGGCA
GCACCGGCCACCTGTACCTGGATGTGAAGAGCAGCTCCAT
GGATGGCAAGAAGACCAACTACGGCCGGGTGGACTTTGCC
ACCTTCGGGGTGTCTGTGTCTGGAAATGCCGCCATCAAGG
GCAGCCCTGTGAAGCTGACAGCCACCGGGGCCAAGGCCTT
TGCTGGCTTCTACAGAGCTGGGGAGCCCATGAACCCCCTG
AGCACCAACCTGACCCTGTCTGCTGAGAAGGTGTCCCACA
ACGTGACTGTGGATGCTGTGACTGGCAAGGTGATTGGAGA
TGATTCTGGCAAGGGGGCCGGCCGGGGCCTGCCTGTGACC

It is emphasized that all sequences above containing a His-tag (e.g. the sequences marked with “His-tag” “yes” in tables 1A and 1B) shall be regarded as being disclosed herein with (as listed above) and without the His-tag (i.e. according to the sequences above but lacking the His residues e.g. at the 6 most C-terminal positions in SEQ ID NOs: 1-3, 8, 22, etc.; or at positions 2 to 7 in SEQ ID NOs: 4-6, 50, etc).

Moreover, all sequences according to the present invention referred to and disclosed herein are to be regarded as being disclosed with or without an N-terminal methionine residue. This residue may be present or not in a polypeptide being recombinantly produced in a given host or in a polypeptide/fragment/derivative according to the present invention as disclosed herein but added due to reasons of recombinant expression. For example, the sequences of the H4 derivatives disclosed herein may be defined as comprising three different Dsa1/DsA2 fragments (S29-L145 of DsA1, G190-T321 of DsA2, T277-T333 of DsA1 and—additionally—a methionine at the N-terminus. However, the first (DsA1) fragment may also be defined as having the original methionine of DsA1 (M1) with a deletion of L2-A28. The same holds true for e.g. all the sequences above and disclosed herein which lack the signal sequence or parts thereof (e.g. for recombinant expression (especially in prokaryotic expression systems or expression systems which do not make use of a natural signal sequence) which start with a methionine (although the adjacent (second) amino acid residue is not naturally located adjacent to an N-terminal methionine. Accordingly, for all sequences referred to and disclosed herein, the N-terminal methionine (if present) may be defined as being the original M1 of DsA1/DsA2/PITP (eventually with a deletion of the amino acid sequence to the second amino acid after the N-terminal methionine) or as an additional methionine added for recombinant expression reasons. Furthermore, a loss/presence of the N-terminal methionine may be caused by the presence of a methionine aminopeptidase (the enzyme which removes methionine, or specifically formylmethionine from the N-terminus: see e.g.: Xiao et al, Biochemistry 49 (2010), 5588-5599). Technically it is also possible that a certain fraction of polypeptides does not contain methionine but formylmethionine, especially in prokaryotic hoat cells. Depending on the specific expression system, this may be the actually initiating amino acid, but it is typically enzymatically altered by enzyme formylmethionine deformylase to standard methionine, which can then be cleaved off (or not; Wingfield, Cuff. Protoc. Protein Sci. 88 (2018), 6.14.1-6.14.3). Both may be cleaved by methionine aminopeptidase. Normally this should be a minor fraction of a recombinant polypeptide lot produced by a fermentation process, but this is a statistical process, so that a certain fraction of polypeptides may start with formylmethionine rather than methionine.

1-32. (canceled)

33. A polypeptide comprising at least a Dermatan sulfate-binding adhesin 1 of P. acnes (DsA1), or a fragment or derivative thereof, and at least a Dermatan sulfate-binding adhesin 2 of P. acnes (DsA2), or a fragment or derivative thereof, said DsA1 and DsA2 comprising from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”);
and, optionally, a Pro Thr repeat containing region (“PT repeat region”), and a C-terminal region “CTR”;
wherein the polypeptide comprises at least CSD1, CSD2 or CSD3 of DsA1, and at least CSD1, CSD2 or CSD3 of DsA2,
wherein the DsA1 domains are defined as follows: NSR: from S29 to I48, CSD1 from 149 to L130, SR1 from G131 to S147, CSD2 from A148 to L267, SR2 from A268 to T277, CSD3 from A278 to K323, the PT repeat region from P324 to T361, and CTR from S362 to F405 in the sequence of Q6A5X9 of the UniProt database (SEQ ID NO: 64);
and wherein the DsA2 domains are defined as follows: NSR: from A72 to K92, CSD1 from 193 to L174, SR1 from S175 to S191, CSD2 from A192 to L311, SR2 from A312 to T321, CSD3 from A322 to E366, the PT repeat region from P367 to T420, and CTR from H421 to A463 in the sequence of Q6A5P9 of the UniProt database (SEQ ID NO: 65).

34. The polypeptide according to claim 33, wherein:
(i) the polypeptide comprises an amino acid exchange at one or more of C53, C319 and C321 of DsA1, and C97 and C363 of DsA2, if present in the polypeptide;
(ii) the polypeptide comprises at least two of CSD1, CSD2 and CSD3 of DsA1 or DsA2;
(iii) the DsA1, or a fragment or derivative thereof, and/or the DsA2, or a fragment or derivative thereof, comprises or consists at least of a CSD2;
(iv) in the fragment or derivative of DsA1 and/or DsA2 at least 5 PT repeats, preferably at least 10 PT repeats, especially at least 15 PT repeats, are deleted compared to a naturally occurring wild type DsA1 or DsA2 polypeptide, and wherein preferably at least one, more preferred at least two, more preferred at least three, even more preferred at least four, especially five, PT repeat(s) is/are present; and/or
(v) the polypeptide comprises or consists of a sequence according to any one of SEO ID NO: 44-50, preferably according to SEQ ID NO: 49, optionally wherein the polypeptide lacks an N-terminal methionine residue.

35. (canceled)

36. A nucleic acid encoding a polypeptide comprising at least a Dermatan sulfate-binding adhesin 1 of P. acnes (DsA1), or a fragment or derivative thereof, and at least a Dermatan sulfate-binding adhesin 2 of P. acnes (DsA2), or a fragment or derivative thereof, said DsA1 and DsA2 comprising from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”); and, optionally a Pro-Thr repeat containing region (“PT repeat region”), and a C-terminal region “CTR”);
wherein the polypeptide comprises at least CSD1, CSD2 or CSD3 of DsA1, and at least CSD1, CSD2 or CSD3 of DsA2,
wherein the DsA1 domains are defined as follows: NSR: from S29 to I48, CSD1 from I49 to L130, SR1 from G131 to S147, CSD2 from A148 to L267, SR2 from A268 to T277, CSD3 from A278 to K323, the PT repeat region from P324 to T361, and CTR from S362 to F405 in the sequence of Q6A5X9 of the UniProt database (SEQ ID NO: 64);
and wherein the DsA2 domains are defined as follows: NSR: from A72 to K92, CSD1 from 193 to L174, SR1 from S175 to S191, CSD2 from A192 to L311, SR2 from A312 to T321, CSD3 from A322 to E366, the PT repeat region from P367 to T420, and CTR from H421 to A463 in the sequence of Q6A5P9 of the UniProt database (SEQ ID NO: 65), optionally wherein the nucleic acid is DNA or RNA (e.g., mRNA).

37. A pharmaceutical composition, e.g. a vaccine, comprising the polypeptide according to claim 33.

38. A polypeptide comprising a putative iron-transport protein of P. acnes (PITP) and/or a fragment or a derivative thereof, wherein PITP comprises from N- to C-terminus an extended neocarzinostatin family domain (“ENFD”), a first swapping region (“SR1”), a heme-binding domain (“HbD”), a second swapping region (“SR2”) including the C-terminal LPXT(G) motif, and a hydrophobic C-terminal region (“hLAR”), wherein the fragment and/or the derivative comprises or consists at least of a PITP epitope,
wherein the PITP domains are defined as follows: ENFD from A32 to R164; SR1 from E165 to K237, HbD from V238 to L396, SR2 (including C-terminal LPXT(G) motif (i.e. including LPXT, but not the G)) from S397 to T430, and hLAR from G431 to 1467 in the sequence of Q6A9N1 of the UniProt database (SEQ ID NO: 66),
optionally wherein
(i) the PITP derivative comprises an amino acid exchange at positions C231 (e.g., C231S) and/or C402 (e.g., C402S), if present in the polypeptide;
(ii) the fragment and/or derivative is a PITP polypeptide wherein the hLAR is deleted, replaced by a hydrophilic C-terminal region, or partially deleted, wherein the partial deletion results in a loss of hLAR except the N-terminal 12 amino acids of hLAR, preferably except the N-terminal 11 amino acids of hLAR, especially except the N-terminal 10 amino acids of hLAR; or a fragment thereof or derivative thereof comprising at least amino acids corresponding to proline 34 to glutamic acid 73 or proline 94 to threonine 143 of ENFD or valine 238 to asparagine 393 of HbD in the amino acid sequence Q6A9N1 of the UniProt database (SEO ID NO: 66); and/or
(iii) the polypeptide comprises or consists of a sequence according to any one of SEO ID NO: 14-20, preferably according to SEO ID NO: 20, optionally wherein the polypeptides lacks an N-terminal methionine residue.

39. (canceled)

40. A nucleic acid encoding a polypeptide comprising a putative iron-transport protein of P. acnes (PITP) and/or a fragment or a derivative thereof, wherein PITP comprises from N- to C-terminus an extended neocarzinostatin family domain (“ENFD”), a first swapping region (“SR1”), a heme-binding domain (“HbD”), a second swapping region (“SR2”) including the C-terminal LPXT(G) motif, and a hydrophobic C-terminal region (“hLAR”), wherein the fragment and/or the derivative comprises or consists at least of a PITP epitope, wherein the PITP domains are defined as follows: ENFD from A32 to R164; SR1 from E165 to K237, HbD from V238 to L396, SR2 (including C-terminal LPXT(G) motif (i.e. including LPXT, but not the G)) from S397 to T430, and hLAR from G431 to 1467 in the sequence of Q6A9N1 of the UniProt database (SEQ ID NO: 66), optionally wherein the nucleic acid is DNA or RNA (e.g., mRNA).

41. A pharmaceutical composition, e.g. a vaccine, comprising the polypeptide according to claim 38.

42. A polypeptide comprising at least a Dermatan sulfate-binding adhesin 1 of P. acnes (DsA1), or a fragment or derivative thereof, and at least a Dermatan sulfate-binding adhesin 2 of P. acnes (DsA2), or a fragment or derivative thereof, said DsA1 and DsA2 comprising from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”);
and, optionally a Pro-Thr repeat containing region (“PT repeat region”), and a C-terminal region (“CTR”);
wherein the polypeptide comprises at least CSD1, CSD2 or CSD3 of DsA1, and at least CSD1, CSD2 or CSD3 of DsA2,
wherein the DsA1 domains are defined as follows: NSR: from S29 to I48, CSD1 from 149 to L130, SR1 from G131 to S147, CSD2 from A148 to L267, SR2 from A268 to T277, CSD3 from A278 to K323, the PT repeat region from P324 to T361, and CTR from S362 to F405 in the sequence of Q6A5X9 of the UniProt database (SEQ ID NO: 64);
and wherein the DsA2 domains are defined as follows: NSR: from A72 to K92, CSD1 from 193 to L174, SR1 from S175 to S191, CSD2 from A192 to L311, SR2 from A312 to T321, CSD3 from A322 to E366, the PT repeat region from P367 to T420, and CTR from H421 to A463 in the sequence of Q6A5P9 of the UniProt database (SEQ ID NO: 65);
and further comprising at least one additional amino acid sequence comprising or consisting of at least one additional P. acnes antigen or P. acnes epitope which is not a DsA1 or DsA2 antigen or epitope, preferably at least one additional amino acid sequence comprising at least the putative iron-transport protein (PITP) polypeptide of P. acnes or a fragment or derivative thereof, wherein the PITP polypeptide comprises from N- to C-terminus an extended neocarzinostatin family domain (“ENFD”), a first swapping region (“SR1”), a heme-binding domain (“HbD”), a second swapping region (“SR2”) including the C-terminal LPXT(G) motif, and a hydrophobic C-terminal region (“hLAR”).

43. The polypeptide according to claim 42, wherein the additional amino acid sequence corresponds to the PITP polypeptide or a fragment or derivative thereof, wherein the fragment or derivative thereof comprises a sequence comprising at least one of ENFD, SR1, HbD, SR2, preferably selected from ENFD and HbD or an epitope containing fragment thereof, more preferred a porphyrin-binding domain thereof, especially wherein the fragment of the PITP polypeptide is selected from the group of the polypeptides consisting of the amino acids A32 to T430, A32 to G426, A32 to Q198, A32 to T143, A32 to K400, A32 to T159, A32 to I177, A32 to Q204, A32 to G234, A32 to R164, A32 to S391, A32 to P179, A32 to R158, A32 to G147, A32 to E73 and P94 to G147; P34 to T430, P34 to G426, P34 to Q198, P34 to T143, P34 to K400, P34 to T159, P34 to I177, P34 to Q204, P34 to G234, P34 to R164, P34 to S391, P34 to P179, P34 to R158, P34 to G147, P34 to E73 and P94 to G147; S240 to S391, A32 to D441, A32 to I440, A32 to E439, A32 to D438, A32 to S437, A32 to S436, A32 to P435, A32 to G434, A32 to E433, A32 to A432, A32 to G431; P34 to D441, P34 to I440, P34 to E439, P34 to D438, P34 to S437, P34 to S436, P34 to P435, P34 to G434, P34 to E433, P34 to A432, P34 to G431; S240 to D441, S240 to I44G, S240 to E439, S240 to D438, S240 to S437, S240 to S436, S240 to P435, S240 to G434, S240 to E433, S240 to A432, S240 to G431; A32 to T430, A32 to V429, A32 to P428, A32 to L427, A32 to G426, A32 to R425, A32 to G424, A32 to A423, A32 to G422, A32 to K421, A32 to G420, A32 to S419, A32 to D418, A32 to D417, A32 to G416, A32 to I415, A32 to V414, A32 to K413, A32 to G412, A32 to T411, A32 to V410, A32 to A409, A32 to D408, A32 to V407, A32 to T406, A32 to V405, A32 to N404, A32 to H403, A32 to C402, A32 to V401, A32 to K400, A32 to E399, A32 to A398, A32 to S397, A32 to L396, A32 to T395, A32 to L394, A32 to N393, A32 to T392; P34 to T430, P34 to V429, P34 to P428, P34 to L427, P34 to G426, P34 to R425, P34 to G424, P34 to A423, P34 to G422, P34 to K421, P34 to G420, P34 to S419, P34 to D418, P34 to D417, P34 to G416, P34 to I415, P34 to V414, P34 to K413, P34 to G412, P34 to T411, P34 to V410, P34 to A409, P34 to D408, P34 to V407, P34 to T406, P34 to V405, P34 to N404, P34 to H403, P34 to C402, P34 to V401, P34 to K400, P34 to E399, P34 to A398, P34 to S397, P34 to L396, P34 to T395, P34 to L394, P34 to N393, P34 to T392; G172 to T430, G172 to V401, G172 to K400, G172 to L396, G172 to N393, A199 to T430, A199 to V401, A199 to K400, A199 to L396, A199 to N393, H223 to T430, H223 to V401, H223 to K400, H223 to L396, H223 to N393, T232 to T430, T232 to V401, T232 to K400, T232 to L396, T232 to N393, G234 to T430, G234 to V401, G234 to K400, G234 to L396, G234 to N393, V238 to T430, V238 to V401, V238 to K400, V238 to L396, V238 to N393, S240 to T430, S240 to V429, S240 to P428, S240 to L427, S240 to G426, S240 to R425, S240 to G424, S240 to A423, S240 to G422, S240 to K421, S240 to G420, S240 to S419, S240 to D418, S240 to D417, S240 to G416, S240 to I415, S240 to V414, S240 to K413, S240 to G412, S240 to T411, S240 to V410, S240 to A409, S240 to D408, S240 to V407, S240 to T406, S240 to V405, S240 to N404, S240 to H403, S240 to C402, S240 to V401, S240 to K400, S240 to E399, S240 to A398, S240 to S397, S240 to L396, S240 to T395, S240 to L394, S240 to N393, S240 to T392; 39I-48I, 40P-49T, 41V-50L, 42G-51S, 43R-52G, 44E-53K, 68P-77N, 69A-78S, 70S-79D, 71V-80K, 72P-81F, 73E-82Y, 74F-83G, 75Y-84Y, 76G-85D, 77N-86P, 78S-87S, 79D-88K, 80K-89D, 81F-90T, 82Y-91T, 83G-92E, 84Y-93S, 85D-94P, 86P-95S, 87S-96T, 88K-97I, 89D-98W, 90T-99V, 91T-100Y, 92E-101T, 93S-102P, 94P-103S, 95S-104Q, 96T-105K, 97I-106A, 98W-107I, 99V-108G, 100Y-109S, 101T-110R, 102P-111F, 103S-112A, 104Q-113Q, 105K-114G, 106A-115R, 107I-116P, 108G-117M, 109S-118N, 110R-119N, 111F-120D, I12A-I21G, I25I-134Q, I26T-135G, I27M-136K, I28K-137D, 144K-153H, 145A-154S, 146H-155D, 147G-156D, 148V-157T, 149G-158R, 150K-159T, 151T-160P, 152D-161V, 153H-162T, 154S-163Y, 155D-164R, 156D-165E, 157T-166A, 158R-167T, 159T-168P, 160P-169A, 161V-170P, 162T-171T, 163Y-172G, 164R-173P, 165E-174K, 166A-175T, 167T-176P, 168P-177I, 169A-178A, 170P-179P, 171T-180S, 172G-181K, 173P-182Q, 174K-183P, 175T-184S, 176P-185K, 177I-186Q, 178A-187A, 179P-188A, 180S-189P, 181K-190S, 182Q-191K, 183P-192Q, 184S-193V, 185K-194K, 186Q-195P, 187A-196S, 188A-197K, 189P-198Q, 190S-199A, 191K-200G, 192Q-201P, 193V-202N, 194K-203K, 195P-204Q, 196S-205S, 197K-206T, 198Q-207T, 199A-208P, 200G-209Q, 201P-210Q, 202N-211K, 203K-212T, 204Q-213A, 205S-214E, 206T-215H, 207T-216R, 208P-217S, 209Q-218Q, 210Q-219T, 211K-220P, 212T-221A, 213A-222A, 214E-223H, 215H-224R, 216R-225T, 217S-226M, 218Q-227T, 219T-228K, 220P-229Q, 221A-230V, 222A-231C, 223H-232T, 224R-233I, 225T-234G, 226M-235A, 227T-236S, 228K-237K, 229Q-238V, 230V-239T, 231C-240S, 232T-241G, 2331-242S, 266L-275S, 267S-276A, 268G-277F, 282T-291K, 334S-343N, 353G-362I, 354V-363K, 355S-364G, 356V-365S, 357S-366P, 358G-367V, 359N-368K, 377F-386P, 378A-387M, 379G-388N, 380F-389P, 396L-405V, 397S-406T, 398A-407V, 399E-408D, 400K-409A, 401V-410V, 402C-411T, 403H-412G, 404N-413K, 405V-414V, 406T-415I, 407V-416G, 408D-417D, 409A-418D, 410V-419S, 411T-420G, 412G-421K, 413K-422G, 414V-423A, 415I-424G, 416G-425R, 417D-426G, 418D-427L, 419S-428P, 420G-429V, 421K-430T, 422G-431G, 423A-432A, 424G-433E, 425R-434G, 426G-435P, 427L-436S, 428P-437S, 429V-438D, 430T-439E, 431G-440I, 432A-441D, 433E-442L, 434G-4430, 435P-444I, 436S-445V, and 437S-446G; 139-K53, P68-G121, I125-D137, K144-S242, L266-F277, T282-K291, S334-N343, G353-K368, F377-P389, and L396-G446, especially a fragment or derivative comprising or consisting of the fragments selected from the group consisting of the PITP fragments A32 to R164, A32 to Q198, A32 to T143, A32 to V148, A32 to T171, P34 to R164, A32 to T159, A32 to I177, A32 to Q204, A32 to G234, A32 to K400, A32 to S391, V238 to K400, A199 to T430, V238 to T395, G234 to K400, H223 to K400, T232 to V401, V238 to T392, V238 to N393, V238 to L394, V238 to T395, V238 to L396, T232 to T430, G172 to K400, and G172 to G234, and fragments thereof comprising at least of a PITP epitope, according to the amino acid numbering in the amino acid sequence Q6A9N1 of the UniProt database (SEQ ID NO: 66).

44. A pharmaceutical composition, e.g. a vaccine, comprising:
(i) a polypeptide comprising at least a Dermatan sulfate-binding adhesin 1 of P. acnes (DsA1), or a fragment or derivative thereof, and at least a Dermatan sulfate-binding adhesin 2 of P. acnes (DsA2), or a fragment or derivative thereof, said DsA1 and DsA2 comprising from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”); and, optionally a Pro-Thr repeat containing region (“PT repeat region”), and a C-terminal region (“CTR”);
wherein the polypeptide comprises at least CSD1, CSD2 or CSD3 of DsA1, and at least CSD1, CSD2 or CSD3 of DsA2,
wherein the DsA1 domains are defined as follows: NSR: from S29 to I48, CSD1 from 149 to L130, SR1 from G131 to S147, CSD2 from A148 to L267, SR2 from A268 to T277, CSD3 from A278 to K323, the PT repeat region from P324 to T361, and CTR from S362 to F405 in the sequence of Q6A5X9 of the UniProt database (SEQ ID NO: 64);
and wherein the DsA2 domains are defined as follows: NSR: from A72 to K92, CSD1 from 193 to L174, SR1 from S175 to S191, CSD2 from A192 to L311, SR2 from A312 to T321, CSD3 from A322 to E366, the PT repeat region from P367 to T420, and CTR from H421 to A463 in the sequence of Q6A5P9 of the UniProt database (SEQ ID NO: 65); and
(ii) a polypeptide comprising a putative iron-transport protein of P. acnes (PITP) or a fragment or a derivative thereof, wherein PITP comprises from N- to C-terminus an extended neocarzinostatin family domain (“ENFD”), a first swapping region (“SR1”), a heme-binding domain (“HbD”), a second swapping region (“SR2”) including the C-terminal LPXT(G) motif, and a hydrophobic C-terminal region (“hLAR”), wherein the fragment and/or the derivative comprises or consists at least of a PITP epitope,
wherein the PITP domains are defined as follows: ENFD from A32 to R164; SR1 from E165 to K237, HbD from V238 to L396, SR2 (including C-terminal LPXT(G) motif (i.e. including LPXT, but not the G)) from S397 to T430, and hLAR from G431 to 1467 in the sequence of Q6A9N1 of the UniProt database (SEQ ID NO: 66).

45. A method of treatment or prevention of a P. acnes-associated infection in a patient, preferably a human patient, comprising administering an effective amount of the polypeptide of claim 33, to a patient in need thereof, optionally wherein the P. acnes-associated infection is:
(i) selected from the group consisting of: acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes; and/or
(ii) a pathological condition associated with any of Type I, II, or III P. acnes, a combination of at least two phylotypes of Type I, II and III, or of at least two ribotypes of P. acnes, especially a P. acnes-associated infection or pathological conditions associated with Type IB, and III of P. acnes.

46. (canceled)

47. A method of production of the polypeptides, claim 33, wherein the polypeptides comprising at least one P. acnes epitope as defined in these claims are expressed in a host cell, extracted and purified from these host cells, and, optionally, formulated and finished to a pharmaceutical formulation, especially a vaccine for use in the treatment or prevention of P. acnes-associated infections in a human patient.

48. A method for producing a vaccine for use in the treatment or prevention of P. acnes-associated infections, comprising
selecting at least one antigen comprising at least one P. acnes epitope, wherein the P. acnes epitope is an epitope which is directly accessible to human serum antibodies when presented by the live P. acnes bacterium and the specific binding of the epitope by the human immune system leads to reduction of bacterial numbers, fitness, growth, virulence or a combination thereof, especially the reduction of bacterial numbers,
wherein binding of the epitope by the immune system, especially by the human immune system, is evidenced by using a flow cytometry assay, preferably by fluorescence-activated cell sorting (FACS) assay; and
wherein reduction of bacterial numbers, fitness, growth, virulence or a combination thereof is evidenced by an opsonophagocytic killing assay (OPK) in the presence of antibodies specific for the at least one antigen comprising at least one P. acnes epitope.

49. A pharmaceutical composition, e.g. a vaccine, comprising the nucleic acid of claim 36.

50. A pharmaceutical composition, e.g. a vaccine, comprising the nucleic acid of claim 40.

51. A nucleic acid encoding the polypeptide of claim 42.

52. A pharmaceutical composition, e.g. a vaccine, comprising a nucleic acid encoding the polypeptide as defined in claim 44(i) and a nucleic acid encoding the polypeptide as defined in claim 44(ii).

53. A Dermatan sulfate-binding adhesin 1 of P. acnes (DsA1 polypeptide) and/or Dermatan sulfate-binding adhesin 2 of P. acnes (DsA2 polypeptide), and/or a fragment and/or a derivative of DsA1 or DsA2, wherein DsA1 and DsA2 comprise from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”), a Pro-Thr repeat containing region (“PT repeat region”), and a C-terminal region (“CTR”),
wherein the DsA1 domains are defined as follows: NSR: from S29 to I48, CSD1 from I49 to L130, SR1 from G131 to S147, CSD2 from A148 to L267, SR2 from A268 to T277, CSD3 from A278 to K323, the PT repeat region from P324 to T361, and CTR from S362 to F405 in the sequence of Q6A5X9 of the UniProt database (SEQ ID NO: 64);
and wherein the DsA2 domains are defined as follows: NSR: from A72 to K92, CSD1 from 193 to L174, SR1 from S175 to S191, CSD2 from A192 to L311, SR2 from A312 to T321, CSD3 from A322 to E366, the PT repeat region from P367 to T420, and CTR from H421 to A463 in the sequence of Q6A5P9 of the UniProt database (SEQ ID NO: 65).

54. A nucleic acid encoding the polypeptide of claim 53, optionally wherein the nucleic acid is DNA or RNA (e.g., mRNA).

55. A pharmaceutical composition, e.g., a vaccine, comprising the polypeptide of claim 53.

56. A pharmaceutical composition, e.g., a vaccine, comprising the nucleic acid of claim 54.

57. A pharmaceutical composition, e.g. a vaccine, comprising at least two antigenic polypeptides with epitopes of P. acnes which are surface exposed, wherein preferably at least one epitope is an epitope of a dermatan sulfate-binding adhesin 1 of P. acnes (DsA1 polypeptide) which is surface exposed or an epitope of a dermatan sulfate-binding adhesin 2 of P. acnes (DsA2 polypeptide) which is surface exposed or an epitope of a putative iron-transport protein of P. acnes (PITP polypeptide) which is surface exposed,
wherein DsA1 and DsA2 comprise from N- to C-terminus an N-terminal swapping region (“NSR”), a first conserved sub-domain (“CSD1”), a first swapping region (“SR1”), a second conserved sub-domain (“CSD2”), a second swapping region (“SR2”), a third conserved sub-domain (“CSD3”); a Pro-Thr repeat containing region (“PT repeat region”), and a C-terminal region (“CTR”),
and wherein PITP comprises from N- to C-terminus an extended neocarzinostatin family domain (“ENFD”), a first swapping region (“SR1”), a heme-binding domain (“HbD”), a second swapping region (“SR2”) including the C-terminal LPXT(G) motif, and a hydrophobic C-terminal region (“hLAR”),
wherein the DsA1 domains are defined as follows: NSR: from S29 to I48, CSD1 from I49 to L130, SR1 from G131 to S147, CSD2 from A148 to L267, SR2 from A268 to T277, CSD3 from A278 to K323, the PT repeat region from P324 to T361, and CTR from S362 to F405 in the sequence of Q6A5X9 of the UniProt database (SEQ ID NO: 64);
wherein the DsA2 domains are defined as follows: NSR: from A72 to K92, CSD1 from 193 to L174, SR1 from S175 to S191, CSD2 from A192 to L311, SR2 from A312 to T321, CSD3 from A322 to E366, the PT repeat region from P367 to T420, and CTR from H421 to A463 in the sequence of Q6A5P9 of the UniProt database (SEQ ID NO: 65), and
wherein the PITP domains are defined as follows: ENFD from A32 to R164; SR1 from E165 to K237, HbD from V238 to L396, SR2 (including C-terminal LPXT(G) motif (i.e. including LPXT, but not the G)) from S397 to T430, and hLAR from G431 to 1467 in the sequence of Q6A9N1 of the UniProt database (SEQ ID NO: 66).

58. A method of treatment or prevention of a P. acnes-associated infection in a patient, preferably a human patient, comprising administering an effective amount of the polypeptide of claim 38, to a patient in need thereof, optionally wherein the P. acnes-associated infection is:
(i) selected from the group consisting of: acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes; and/or
(ii) a pathological condition associated with any of Type I, II, or III P. acnes, a combination of at least two phylotypes of Type I, II and III, or of at least two ribotypes of P. acnes, especially a P. acnes-associated infection or pathological conditions associated with Type IB, and III of P. acnes.

59. A method of treatment or prevention of a P. acnes-associated infection in a patient, preferably a human patient, comprising administering an effective amount of the polypeptide according to claim 42, to a patient in need thereof, optionally wherein the P. acnes-associated infection is:
(i) selected from the group consisting of: acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes; and/or
(ii) a pathological condition associated with any of Type I, II, or III P. acnes, a combination of at least two phylotypes of Type I, II and III, or of at least two ribotypes of P. acnes, especially a P. acnes-associated infection or pathological conditions associated with Type IB, and III of P. acnes.

60. A method of treatment or prevention of a P. acnes-associated infection in a patient, preferably a human patient, comprising administering an effective amount of the DsA1 polypeptide and/or the DsA2 polypeptide, and/or a fragment and/or a derivative of DsA1 or DsA2 of claim 53, to a patient in need thereof, optionally wherein the P. acnes-associated infection is:
(i) selected from the group consisting of: acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes; and/or
(ii) a pathological condition associated with any of Type I, II, or III P. acnes, a combination of at least two phylotypes of Type I, II and III, or of at least two ribotypes of P. acnes, especially a P. acnes-associated infection or pathological conditions associated with Type IB, and III of P. acnes.

61. A method of treatment or prevention of a P. acnes-associated infection in a patient, preferably a human patient, comprising administering an effective amount of the pharmaceutical composition of claim 57, to a patient in need thereof, optionally wherein the P. acnes-associated infection is:
(i) selected from the group consisting of: acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes; and/or
(ii) a pathological condition associated with any of Type I, II, or III P. acnes, a combination of at least two phylotypes of Type I, II and III, or of at least two ribotypes of P. acnes, especially a P. acnes-associated infection or pathological conditions associated with Type IB, and III of P. acnes.

62. A method of production of the polypeptide of claim 38, wherein the polypeptides comprising at least one P. acnes epitope as defined in these claims are expressed in a host cell, extracted and purified from these host cells, and, optionally, formulated and finished to a pharmaceutical formulation, especially a vaccine for use in the treatment or prevention of P. acnes-associated infections in a human patient.

63. A method of production of the polypeptide of claim 42, wherein the polypeptides comprising at least one P. acnes epitope as defined in these claims are expressed in a host cell, extracted and purified from these host cells, and, optionally, formulated and finished to a pharmaceutical formulation, especially a vaccine for use in the treatment or prevention of P. acnes-associated infections in a human patient.

64. A method of production of the polypeptide of claim 53, wherein the polypeptides comprising at least one P. acnes epitope as defined in these claims are expressed in a host cell, extracted and purified from these host cells, and, optionally, formulated and finished to a pharmaceutical formulation, especially a vaccine for use in the treatment or prevention of P. acnes-associated infections in a human patient.

65. A method of treatment or prevention of a P. acnes-associated infection in a patient, preferably a human patient, comprising administering an effective amount of a nucleic acid encoding the polypeptide of claim 33 to a patient in need thereof, optionally wherein the P. acnes-associated infection is:
(i) selected from the group consisting of: acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes; and/or
(ii) a pathological condition associated with any of Type I, II, or III P. acnes, a combination of at least two phylotypes of Type I, II and III, or of at least two ribotypes of P. acnes, especially a P. acnes-associated infection or pathological conditions associated with Type IB, and III of P. acnes.

66. A method of treatment or prevention of a P. acnes-associated infection in a patient, preferably a human patient, comprising administering an effective amount of a nucleic acid encoding the polypeptide of claim 38, to a patient in need thereof, optionally wherein the P. acnes-associated infection is:
(i) selected from the group consisting of: acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes; and/or
(ii) a pathological condition associated with any of Type I, II, or III P. acnes, a combination of at least two phylotypes of Type I, II and III, or of at least two ribotypes of P. acnes, especially a P. acnes-associated infection or pathological conditions associated with Type IB, and III of P. acnes.

67. A method of treatment or prevention of a P. acnes-associated infection in a patient, preferably a human patient, comprising administering an effective amount of a nucleic acid encoding the polypeptide according to claim 42, to a patient in need thereof, optionally wherein the P. acnes-associated infection is:
(i) selected from the group consisting of: acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes; and/or
(ii) a pathological condition associated with any of Type I, II, or III P. acnes, a combination of at least two phylotypes of Type I, II and III, or of at least two ribotypes of P. acnes, especially a P. acnes-associated infection or pathological conditions associated with Type IB, and III of P. acnes.

68. A method of treatment or prevention of a P. acnes-associated infection in a patient, preferably a human patient, comprising administering an effective amount of a nucleic acid encoding the DsA1 polypeptide and/or the DsA2 polypeptide, and/or a fragment and/or a derivative of DsA1 or DsA2 of claim 53, to a patient in need thereof, optionally wherein the P. acnes-associated infection is:
(i) selected from the group consisting of: acne vulgaris, keratitis, synovitis acne pustulosis hyperostosis osteitis (SAPHO) syndrome, endocarditis, prosthetic joint infections, surgical wound infections, vascular graft infections, anaerobic arthritis, cardiovascular device-related infections, such as prosthetic valve endocarditis; ocular implant infections, breast implant illness, sciatica, conjunctivitis, shunt-associated and/or spinal hardware central nervous system infections, shunt-associated central nervous system infections, sarcoidosis, endophthalmitis osteomyelitis, allergic alveolitis, rheumatoid arthritis, infectious arthritis, chronic juvenile arthritis, chronic destructive oligoarthritis, degenerative disc disease, dental infections, ulcerative colitis hyperpyrexia, cerebral abscess, subdural empyema, peritonitis, periodontitis, endodontic infections, endophthalmitis, keratitis, chronic rhinosinusitis, folliculitis, keratitis, corneal ulcer, endophthalmitis, prostate inflammation, chronic prostatitis, primary biliary cirrhosis, hidradenitis suppurativa, pulmonary angitis, acne inversa, progressive macular hypomelanosis, acne conglobata, atherosclerosis, prostatic cancer, and a medical implant biofilm infection by P. acnes; and/or
(ii) a pathological condition associated with any of Type I, II, or III P. acnes, a combination of at least two phylotypes of Type I, II and III, or of at least two ribotypes of P. acnes, especially a P. acnes-associated infection or pathological conditions associated with Type IB, and III of P. acnes.
